(19) United States
(12) Patent Application Publication
(10) Pub. No.: US 2022/0257686 A1
(43) Pub. Date: Aug. 18, 2022

(54) COMPOSITIONS AND METHODS TO AFFECT HUMAN GUT MICROBES

(71) Applicants: Washington University, St. Louis, MO (US); Intercontinental Great Brands LLC, East Hanover, NJ (US)

(72) Inventors: MICHAEL PATNODE, St. Louis, MO (US); ZACHARY BELLER, St. Louis, MO (US); NATHAN HAN, St Louis, MO (US); DARRYL WESENER, St. Louis, MO (US); OMAR DELANNOY-BRUNO, St. Louis, MO (US); SOPHIE VINOY, St. Louis, MO (US); JEFFREY GORDON, St. Louis, MO (US); DAVID KAY HAYASHI, St. Louis, MO (US); ALEXANDRA MEYNIER, St. Louis, MO (US); MONIKA OKONIEWSKA, St. Louis, MO (US); VANI VEMULAPALLI, St. Louis, MO (US); MICHAEL BARRATT, St. Louis, MO (US)

(21) Appl. No.: 17/628,491

(22) PCT Filed: Jul. 17, 2020
(86) PCT No.: PCT/US20/42669

§ 371 Date: Jan. 19, 2022

Related U.S. Application Data
(60) Provisional application No. 62/876,388, filed on Jul. 19, 2019.

(51) Int. Cl. A61K 36/48; A23L 33/22; A61K 36/752; A61K 31/732; A61K 36/8998; A61K 31/733; A61P 1/00

(52) U.S. Cl. CPC A61K 36/48; A23L 33/22; A61K 36/752; A23V 2002/00; A61K 36/8998; A61K 31/733; A61P 1/00; A61K 31/732

(57) ABSTRACT

The present disclosure provides compositions and foods that selectively promote the representation and expressed beneficial function of members of a human gut community in ways that promote a healthy gut microbiota and in turn positively impact health. Various examples of compositions and foods, which comprise one or more fiber preparation, are discussed in detail, as are methods of their use.

### CROSS REFERENCE TO RELATED APPLICATION

This application claims priority to U.S. Provisional Application No. 62/876,388, filed Jul. 19, 2019, the disclosures of which are incorporated herein by reference.

### GOVERNMENTAL RIGHTS

This invention was made with government support under DK070977, DK078669 and DK107158 awarded by the National Institutes of Health. The government has certain rights in the invention.

### BACKGROUND OF THE INVENTION

Increasing evidence that the gut microbiota impacts multiple features of human biology has catalyzed efforts to develop microbiota-directed interventions that improve health status. Microbiota-directed foods (MDFs) are one approach, as diet has pronounced and rapid effects on microbial community configuration. Dietary carbohydrates provide an important source of energy for gut bacteria, with the products of their metabolism benefiting primary microbial consumers, their syntrophic partners, and the host. Consumption of plant polysaccharides in the form of dietary fiber has been linked to a number of health benefits. In addition, the diminished diversity of complex polysaccharides in the diets of those living in industrialized countries has been associated with loss of bacterial diversity in their microbiota.

From the perspective of gut microbiota, plant material and fiber preparations prepared from plant material contain active and inactive fractions with different structural features and biophysical availability. Historically, identifying the bioactive components of fiber preparations has been a formidable challenge. Accordingly, there remains a need in the art for compositions that selectively promote the representation and expressed beneficial function of members of a human gut community in ways that promote a healthy gut microbiota and in turn positively impact health.

### SUMMARY OF THE INVENTION

In an aspect, the present disclosure encompasses a composition comprising a plurality of fiber preparations, each fiber preparation independently selected from the group consisting of a barley fiber preparation or a glycan equivalent thereof, a citrus fiber preparation or a glycan equivalent thereof, a citrus pectin formulation or a glycan equivalent thereof, a high molecular weight inulin preparation or a glycan equivalent thereof, a pea fiber preparation or a glycan equivalent thereof, and a sugar beet fiber preparation or a glycan equivalent thereof, wherein the plurality of fiber preparations is at least 95 wt % of the composition.

In another aspect, the present disclosure encompasses a composition at least 15 wt % of one or more sugar beet fiber preparation and at least 28 wt % of one or more high molecular weight inulin preparation, and optionally one or more citrus pectin preparation in an amount that does not exceed 10 wt %, one or more citrus fiber preparation in an amount that does not exceed 25 wt %, and one or more barley fiber preparations in an amount does not exceed 45 wt %, wherein the plurality of fiber preparations is at least 95 wt % of the composition.

In another aspect, the present disclosure encompasses a composition comprising at least 15 wt % of one or more pea fiber preparation or a glycan equivalent thereof; and at least one additional fiber preparation chosen from (i) at least 28 wt % of one or more high molecular weight inulin preparation or a glycan equivalent thereof, (ii) 10 wt % or less of one or more citrus pectin preparation or a glycan equivalent thereof, (iii) 25 wt % or less of one or more citrus fiber preparation or a glycan equivalent thereof, or (iv) 45 wt % or less of one or more barley fiber preparation or a glycan equivalent thereof.

In another aspect, the present disclosure encompasses a composition comprising about 35 wt % of one or more pea fiber preparation or a glycan equivalent thereof, about 10 wt % of one or more citrus fiber preparation or a glycan equivalent thereof, about 35 wt % of one or more high molecular weight inulin preparation or a glycan equivalent thereof, and about 20 wt % of one or more barley fiber preparation or a glycan equivalent thereof; and wherein the pea fiber preparation(s), citrus fiber preparation(s), high molecular weight inulin preparation(s), and barley fiber preparation(s) are at least 95 wt % of the composition.

In another aspect, the present disclosure encompasses a composition comprising about 30-40 wt % of one or more pea fiber preparation or a glycan equivalent thereof, about 9-11 wt % of one or more citrus fiber preparation or a glycan equivalent thereof, about 30-40 wt % of one or more high molecular weight inulin or a glycan equivalent thereof, and about 18-22 wt % of one or more barley fiber preparation or a glycan equivalent thereof; and wherein the pea fiber preparation(s), citrus fiber preparation(s), high molecular weight inulin preparation(s), and barley fiber preparation(s) are at least 95 wt % of the composition.

In another aspect, the present disclosure encompasses a composition comprising about 30-35 wt % of one or more pea fiber preparation or a glycan equivalent thereof, about 9-11 wt % of one or more citrus fiber preparation or a glycan equivalent thereof, about 35-40 wt % of one or more high molecular weight inulin preparation or a glycan equivalent thereof, and about 18-22 wt % of one or more barley bran preparation or a glycan equivalent thereof; and wherein the pea fiber preparation(s), citrus fiber preparation(s), high molecular weight inulin preparation(s), and barley fiber preparation(s) are at least 95 wt % of the composition.

In another aspect, the present disclosure encompasses a composition comprising about 33 wt % of one or more pea fiber preparation or a glycan equivalent thereof, about 11 wt % of one or more citrus fiber preparation or a glycan equivalent thereof, about 36 wt % of one or more high molecular weight inulin preparation or a glycan equivalent thereof, and about 20 wt % of one or more barley fiber preparation or a glycan equivalent thereof; and wherein the pea fiber preparation(s), citrus fiber preparation(s), high molecular weight inulin preparation(s), and barley fiber preparation(s) are at least 95 wt % of the composition.

In another aspect, the present disclosure encompasses a composition comprising about 65 wt % pea fiber or a glycan equivalent thereof, and about 35 wt % high molecular weight inulin or a glycan equivalent thereof; and wherein the pea fiber preparation(s) and high molecular weight inulin preparation(s) are at least 95 wt % of the composition.

In another aspect, the present disclosure encompasses food compositions comprising compositions disclosed herein. In some embodiments, the amount of the composition is about 40 wt % to about 50 wt % of the food composition. In some embodiments, the composition provides about 90% or more of the total dietary fibers in the food composition.

In another aspect, the present disclosure encompasses a pressed, extruded or baked food composition, the food composition comprising about 40 wt % to about 95 wt % of a composition of fiber preparations, the composition of fiber preparations comprising (a) about 25 wt % to about 40 wt % of one or more pea fiber preparation, or a glycan equivalent thereof; about 5 wt % to about 15 wt % of one or more citrus fiber preparation, or a glycan equivalent thereof; about 30 wt % to about 40 wt % of one or more high molecular weight inulin preparation, or a glycan equivalent thereof; and about 10 wt % to about 30 wt % of one or more barley fiber preparation, or a glycan equivalent thereof; or (b) about 55 wt % to about 65 wt % of one or more pea fiber preparation, or a glycan equivalent thereof; and about 30 wt % to about 40 wt % of one or more high molecular weight inulin preparation, or a glycan equivalent thereof; wherein a 30 g serving of the food composition has at least 6 g of total dietary fiber; and wherein the food composition effects an increase in the fiber degrading capacity of a subject's gut microbiota and/or an improvement in the a subject's health, when the subject has consumed the food composition at least once a day for at least 5 days (e.g., at least 6 days, at least 7 days, etc.).

Other aspects and iterations of the invention are described more thoroughly below.

### BRIEF DESCRIPTION OF THE FIGURES

The application file contains at least one photograph executed in color. Copies of this patent application publication with color photographs will be provided by the Office upon request and payment of the necessary fee.

FIG. 1A and FIG. 1B show the design and results of an in vivo screen of the effects of food-grade fiber preparations on members of a defined human gut microbiota. FIG. 1A includes a schematic design of the screen (one of three similar screens). Individually-housed adult germ-free mice were colonized with a consortium of 20 bacterial strains obtained from a single human donor. Animals received a series of supplemented HiSF-LoFV diets, each containing one fiber preparation at 8% (w/w) and another at 2% (w/w) (colored boxes). Fecal samples were collected during the last two days of each week-long diet period. Control animals received the unsupplemented HiSF-LoFV or LoSF-HiFV diet monotonously for four weeks. Also shown are the average relative abundance values for B. thetaiotaomicron and B. caccae on days 6 and 7 of treatment with the indicated fiber-supplemented HiSF-LoFV diets. Bars show mean values. Circles denote individual mice. Black arrows point to data obtained from different mice consuming diets containing 8% (w/w) pea fiber consumed at the indicated periods of their diet oscillation sequence. Green arrowheads in panel B mark mice that received pea fiber as the minor fiber type (2% w/w) while purple arrowheads in panel C highlight animals where high molecular weight (MW) inulin was the minor fiber. See Table A for compositional analysis of the 34 fibers. FIG. 1B depicts estimates of coefficients from linear models for bacterial strains across the three screening experiments where models produced at least one estimated coefficient>0.4. Statistically significant coefficients (P<0.01; ANOVA) are shaded according to the color bar.

FIG. 2A, FIG. 2B, FIG. 2C, FIG. 2D, FIG. 2E, FIG. 2F, FIG. 2G, FIG. 2H, FIG. 2I and FIG. 2J show the results of proteomics and forward genetic experiments to identify arabinan in pea fiber as a nutrient source for multiple bacterial species. FIG. 2A is a schematic representation of polysaccharide structures detected in pea fiber based on monosaccharide and linkage analyses (with stereochemistry of anomeric carbon inferred). FIG. 2B-FIG. 2E are graphs showing relative abundance of the indicated bacterial strains. Adult C57BL/6J germ-free mice were colonized with a 15-member community consisting of INSeq libraries representing four Bacteroides species together with 10 additional bacterial strains used in screening experiments depicted in FIG. 1. Relative abundance is shown for each bacterial strain at each of the indicated time points in mice monotonously fed the control HiSF-LoFV diet (grey), or the HiSF-LoFV diet supplemented with 10% (w/w) pea fiber (green). Circles denote individual mice. Shading denotes the 95% Cl. The position of the line within the data points for a given time point represents the mean value (n=15 individually-caged mice per group; Tables S4A-S4C). *, P<0.05; (pea fiber supplemented versus unsupplemented HiSF-LoFV diet; ANOVA). FIG. 2F-FIG. 2I are graphs showing Proteomic and INSeq analyses of fecal samples collected on experimental day 6. On the x-axis, the position of each dot denotes the mean value for the abundance of a single bacterial protein in samples obtained from animals monotonously fed the pea fiber-supplemented HiSF-LoFV diet (relative to controls fed the unsupplemented diet). The y-axis indicates the mean value for the differential enrichment of mutant strains with Tn disruptions in the gene encoding each protein in the pea fiber versus HiSF-LoFV diet groups. The total number of genes represented in both the protein dataset and INSeq mutant pool is shown in the upper left of each plot, and these genes are plotted as grey dots. Green circles highlight genes that are significantly affected by pea fiber (P<0.05, |fold change|>log 2(1.2); limma or limma-voom) as judged by levels of their protein products or their contribution to fitness; open circles mark the subset of these genes that are encoded by PULs. Genes that are present in three homologous arabinan-processing PULs in B. thetaiotaomicron, B. cellulosilyticus, and B. vulgatus are labeled with their PUL number as it appears in PULDB (Terrapon et al., 2018). Genes in an arabinose-processing operon in B. vulgatus are labeled with an CA′. Genes in the B. ovatus RGI-processing PUL97 are also labeled. (J) Alignment of B. thetaiotaomicron PUL7, B. cellulosilyticus PUL5, B. vulgatus PUL27, and the B. vulgatus arabinose operon. The direction of transcription is left to right (unless marked by a leftward pointing arrowhead). The first and last genes are labeled above with their locus tag number. Genes are color-coded according to their functional annotation (see key). GH families for enzymes in the CAZy database are shown as numbers inside the gene boxes (characterized members of GH51, GH43:4, GH43:29, and GH146 are predominantly arabinanases or arabinofuranosidases). Shaded regions connecting genes denote significant BLAST homology (E-value<10⁻⁹); the percent amino acid identity of their protein products is shown.

FIG. 3A, FIG. 3B, FIG. 3C, and FIG. 3D show results from experiments that deliberately manipulate a community composition to demonstrate interspecies competition for pea fiber arabinan. FIG. 3A and FIG. 3C are graphs showing relative abundance of the indicated bacterial strains. Adult C57BL/6J germ-free mice were colonized with the same defined community that was used for the experiments in FIG. 2, with or without B. cellulosilyticus (B.c.). Relative abundance of each bacterial strain is shown at each time point in mice fed the control HiSF-LoFV diet in the presence (light grey, closed circles), or absence (dark grey, open circles) of B. cellulosilyticus, or fed the HiSF-LoFV diet supplemented with 10% (w/w) pea fiber in the presence (green, closed circles) or absence (magenta, open circles) of B. cellulosilyticus. Key: circles, individual mice; lines, mean values; shading, 95% Cl (n=4-10 mice per group). *, P<0.05; (diet-by-community interaction; ANOVA). FIG. 3B and FIG. 3D are plots showing mean values±SD (vertical shading) (n=5 animals/treatment group) from proteomics analysis of fecal communities sampled on experimental days 6, 12, 19, and 25. Genes in PULs of interest are shown along the x-axis (as locus tag number only; BT_XXXX or BVU_XXXX). Genes are color-coded according to their functional annotation (see key). GH families for enzymes in the CAZy database are shown as numbers inside the gene boxes. Key for circles is identical to that used in panels A and C. *, P<0.05, |fold change|>log 2(1.2) (pea fiber supplemented versus unsupplemented HiSF-LoFV diet; limma).

FIG. 4A, FIG. 4B, FIG. 4C, FIG. 4D, and FIG. 4E show results from experiments to characterize glycan processing as a function of community membership with artificial food particles. FIG. 4A is a schematic depiction of a bead-based in vivo glycan degradation assay. FIG. 4B depicts flow cytometry plots showing levels of fluorescence in a pool of three bead types before and after transit though the guts of mice representing two colonization conditions. Axes are labeled with the fluorophore detected in each channel. FIG. 4C graphically depicts the mass of arabinose associated with two types of polysaccharide-coated beads together with empty uncoated beads before (black) and after (green) passage through the intestine of gnotobiotic mice, mono-colonized with either B. cellulosilyticus or B. vulgatus. Beads were purified from cecal and colonic contents four hours after gavage. The mass of arabinose associated with beads is plotted before (black) and after (green) passage through the intestine. Circles denote individual animals. Bars show mean values and 95% Cl. FIG. 4D and FIG. 4E graphically depict polysaccharide degradation in mice colonized with the 15-member community (with B. cellulosilyticus), or the 14-member community (lacking B. cellulosilyticus) fed the HiSF-LoFV diet. The mass of bead-associated arabinose (panel D) or glucose (panel E) is plotted before (black) and after collection from cecal and colonic contents on experimental day 12 (grey, 15-member community group; magenta, minus B. cellulosilyticus group). The presence or absence of B. cellulosilyticus in each group of mice is noted along the x-axis. Circles denote individual mice. Mean values+95% Cl are shown (n=3-6 animals/group). *, P<0.05 (Mann-Whitney U test).

FIG. 5A, FIG. 5B, FIG. 5C, FIG. 5D, FIG. 5E, and FIG. 5F show the results of experiments to detect acclimation to the presence of a potential competitor using proteomics and forward genetics. FIG. 5A and FIG. 5B graphically depict the relative abundance of the indicated bacterial strains after adult C57BL/6J germ-free mice were colonized with the same defined community used for the experiments in FIG. 2, with or without B. cellulosilyticus (B.c.) or B. vulgatus (B.v.). Relative abundance of each bacterial strain in fecal samples is shown at each time point in mice colonized with the 15-member community (grey closed circles) or that community lacking B. cellulosilyticus or B. vulgatus (open circles; magenta and brown respectively). All mice received the control base HiSF-LoFV diet. Key: circles, individual mice; lines, mean values; shading, 95% Cl. FIG. 5C and FIG. 5D are plots showing protein abundance and INSeq data for genes in arabinoxylan PULs shown along the x-axis (as locus tag number only; Bovatus_0XXXX) according to the order in which they appear in the genome. Mean values±SD (vertical shading) are indicated (n=5 animals/treatment group). Genes are color-coded according to functional annotation. Key for circles: grey, 15-member community; magenta or brown, mice harboring communities without B. cellulosilyticus or B. vulgatus, respectively. *, P<0.05, |fold change|>log 2(1.2) [15-member community versus 14-member (minus B. cellulosilyticus); limma or limma-voom]. FIG. 5E is a plot showing a proteomics analysis of fecal communities sampled on experimental day 6. Proteins whose abundances increase significantly in the absence of B. cellulosilyticus appear in the upper right; those encoded by genes in PULs are highlighted with open circles while those encoded by genes in arabinoxylan processing PULs are labeled with their PUL number. FIG. 5F is a plot showing an INSeq analysis showing the change in abundance of mutant strains from experimental day 2 to day 6 relative to the 15-strain community. Genes that are significantly more important for fitness in the absence of B. cellulosilyticus appear in the upper left. Genes in PULs that have a significant effect on fitness are highlighted with open circles; those located in arabinoxylan processing PULs are labeled with their PUL number.

FIG. 6A, FIG. 6B, FIG. 6C, FIG. 6D, FIG. 6E, FIG. 6F, and FIG. 6G show the results of experiments to alleviate competition between arabinoxylan consuming Bacteroides. FIG. 6A, FIG. 6B, and FIG. 6C graphically depict the relative abundance of bacterial strains after adult C57BL/6J germ-free mice were colonized with the same defined community used for the experiments in FIG. 2, with or without B. cellulosilyticus (B.c.) and/or B. ovatus (B.v.). The relative abundance of each bacterial strain is shown at each time point in mice fed the control HiSF-LoFV diet and colonized with the 15-member community (closed circles) or the derivative communities lacking B. cellulosilyticus or B. ovatus or both species (open circles; magenta, orange, and cyan respectively). Key: circles, individual mice; lines, mean values; shading, 95% Cl. FIG. 6D and FIG. 6E graphically show the analysis of B. ovatus or B. cellulosilyticus protein abundances in fecal samples obtained on experimental day 6. Genes in arabinoxylan-processing PULs are shown along the x-axis (as locus tag number only; Bovatus_0XXXX (or BcellWH2_0XXXX) according to the order in which they appear in the genome. Mean values±SD (vertical shading) are indicated (n=5-7 animals/treatment group). Genes are color-coded according to functional annotation (see key). Key for circles: grey, 15-member community; magenta, orange, or cyan, mice harboring communities without B. cellulosilyticus, B. ovatus, or both species, respectively. *, P<0.05 [15-member community versus 14-member (minus B. cellulosilyticus); limma]. FIG. 6F and FIG. 6G graphically show the results of a bead-based assay of polysaccharide degradation in mice fed the HiSF-LoFV diet and colonized with the complete 15-member community, or a community lacking B. cellulosilyticus, B. ovatus, or both species. The mass of bead-associated arabinose (FIG. 6F) or mannose (FIG. 6G) is plotted before (black) and after exposure to the indicated communities (grey, complete 15-member community; magenta, community with B. cellulosilyticus omitted; orange, community lacking B. ovatus; cyan, community lacking both Bacteroides species). The presence or absence of B. cellulosilyticus and B. ovatus in each group of mice is noted along the x-axis. Circles denote individual mice. Mean values+95% Cl are shown (n=5-7 animals/group). *, P<0.05 (Mann-Whitney U test).

FIG. 7A, FIG. 7B, FIG. 7C, FIG. 7D, FIG. 7E, FIG. 7F, FIG. 7G, FIG. 7H, and FIG. 7I show the results of proteomics and forward genetic experiments to identify homogalacturonan in citrus pectin as a nutrient source for multiple bacterial species. FIG. 7A is a schematic representation of polysaccharide structures detected in citrus pectin based on monosaccharide and linkage analyses (with stereochemistry of anomeric carbons inferred). FIG. 7B-E are graphs showing relative abundance of the indicated bacterial strains. Adult C57BL/6J germ-free mice were colonized with a 15-member community consisting of INSeq libraries representing four Bacteroides species together with 10 additional bacterial strains used in screening experiments depicted in FIG. 1. Relative abundance is shown for each bacterial strain at each of the indicated time points in mice fed the control HiSF-LoFV diet (grey), or the HiSF-LoFV diet supplemented with 10% (w/w) citrus pectin (blue). Circles denote individual mice, lines the mean value and shading the 95% Cl (n=15 individually caged mice per group; results pooled from three independent experiments). *, P<0.05 (Mann-Whitney U test). FIG. F-I are plots showing proteomic and INSeq analyses of fecal samples collected on experimental day 6. On the x-axis, each dot denotes the mean value for the abundance of a single bacterial protein in samples from animals monotonously fed the citrus pectin-supplemented HiSF-LoFV diet (relative to controls fed the unsupplemented diet). The y-axis indicates the mean value for the differential enrichment of mutants with Tn disruptions in the gene encoding each protein in the citrus pectin versus HiSF-LoFV diet groups. Blue dots represent genes that are significantly affected by citrus pectin (P<0.05, |fold change|>log 2(1.2); limma or limma-voom) as judged by levels of their protein products or their contribution to fitness while open circles mark the subset of these genes that are encoded by PULs. Genes present in predicted homogalacturonan-processing PULs in B. thetaiotaomicron, B. cellulosilyticus, and B. vulgatus are labeled with their PUL number as it appears in PULDB (Terrapon et al., 2018).

FIG. 8A, FIG. 8B, FIG. 8C, and FIG. 8D show results from experiments that deliberately manipulate a community composition to demonstrate interspecies competition for homogalacturonan in citrus pectin. FIG. 8A and FIG. 8B are graphs showing relative abundance of the indicated bacterial strains. Adult C57BL/6J germ-free mice were colonized with the same defined community that was used for the experiments in FIG. 2, with or without B. cellulosilyticus (B.c.). Relative abundance of each bacterial strain is shown at each time point in mice fed the control HiSF-LoFV diet in the presence (light grey, closed circles), or absence (dark grey, open circles) of B. cellulosilyticus, or fed the HiSF-LoFV diet supplemented with 10% (w/w) citrus pectin in the presence (green, closed circles) or absence (magenta, open circles) of B. cellulosilyticus. Key: circles, individual mice; lines, mean values; shading, 95% Cl (n=4-10 mice per group). *, P<0.05; (diet-by-community interaction; ANOVA). FIG. 8C and FIG. 8D are plots showing mean values±SD (vertical shading) (n=5 animals/treatment group) from proteomics analysis of fecal communities sampled on experimental days 6, 12, 19, and 25. Genes in predicted homogalacturonan-processing PULs are shown along the x-axis (as locus tag number only; BT_XXXX, (BVU_XXXX) according to the order in which they appear in the genome. Mean values±SD (vertical lines) are indicated (n=5 animals/treatment group). Genes are color-coded according to functional annotation (see key). GH families for enzymes in the CAZy database are shown as numbers inside the gene boxes. Key for circles is identical to that used in panels A and B. *, P<0.05, |fold change|>log 2(1.2) (citrus pectin supplemented versus unsupplemented-HiSF-LoFV diet; limma).

FIG. 9A, FIG. 9B, FIG. 9C, FIG. 9D, FIG. 9E, FIG. 9F show results from experiments to characterize glycan processing as a function of community membership with artificial food particles. FIG. 9A and FIG. 9B graphically depict the mass of arabinose or glucose associated with three types of polysaccharide-coated beads or with empty uncoated beads. Gnotobiotic mice, mono-colonized with either B. cellulosilyticus or B. vulgatus, were gavaged with three types of polysaccharide-coated beads together with empty uncoated beads. Beads were purified from cecal and colonic contents 4 hours after gavage. The mass of arabinose (FIG. 9A) or glucose (FIG. 9B) associated with beads is plotted before (black) and after (green) their transit through the gut. Circles denote individual animals. Bars show mean values with 95% Cl. In FIG. 9C and FIG. 9D, adult C57BL/6J germ-free mice were gavaged with four beads types (labeled on the x-axis). Beads were isolated from fecal samples collected from 4 to 12 hours after gavage. The mass of arabinose (FIG. 9C) and glucose (FIG. 9D) associated with beads is plotted before (black) and after (blue) their transit through the gut. Circles denote individual mice. Bars show the mean values+95% Cl (n=13 animals). FIG. 9E and FIG. 9F graphically depict polysaccharide degradation in mice colonized with the 15-member community (with B. cellulosilyticus), or the 14-member community lacking B. cellulosilyticus fed the HiSF-LoFV diet+10% pea fiber. Beads were recovered from cecal and colonic contents. The mass of bead-associated arabinose (FIG. 9E) or glucose (FIG. 9F) is plotted before (black) and after transit through the gut (green, 15-member community group; magenta, minus B. cellulosilyticus group). In FIGS. 9A, B, E, and F, and in FIGS. 4D and 4E, input beads are shared for all plots, since all six groups of mice were analyzed in the same experiment. The presence or absence of B. cellulosilyticus in each group of mice is noted along the x-axis. Circles denote individual mice. Mean values+95% Cl are shown (n=3-6 animals/group). *, P<0.05 (Mann-Whitney U test).

FIG. 10 graphically depicts the results of an adhesion assay using glycan-coated beads and gut microorganisms. The extent of fluorescence (Syto-60+) on the y-axis is measured relative to control beads that are incubated with fluorescent dye but not bacteria.

FIG. 11A, FIG. 11B, FIG. 11C, FIG. 11D, and FIG. 11E illustrate various experimental designs described in the examples. FIG. 11A—Monotonous feeding of the unsupplemented HiSF-LoFV diet or the diet supplemented with one of four different fiber preparations. Fecal samples were collected on days 2, 3, 6, 8, 12, 14, 19 and 21. FIG. 11B—Monotonous feeding of the unsupplemented HiSF-LoFV diet or the HiSF-LoFV diet supplemented with pea fiber or citrus pectin to mice colonized with the community with or without B. cellulosilyticus. Fecal samples were collected on days 2, 3, 6, 8, 12, 14, 19 and 25. FIG. 11C—Monotonous feeding of the HiSF-LoFV with or without pea fiber to mice colonized with the community with or without B. cellulosilyticus. Fecal samples were collected on days 2, 3, 4, 6, 7, 8, 10, 11, and 12. FIG. 11D—Monotonous feeding of HiSF-LoFV with or without citrus pectin to mice colonized with a community with or without B. cellulosilyticus or B. vulgatus. Fecal samples were collected on days 2, 3, 4, 6, 7, 8, 10, and 12. FIG. 11E—Monotonous feeding of the unsupplemented HiSF-LoFV diet to mice harboring communities with or without B. cellulosilyticus and/or B. ovatus. Fecal samples were collected on days 2, 3, 4, 6, 7, 8, and 10.

FIG. 12 is a chemical reaction schematic. Although only a single polysaccharide is used in this depiction, any glycan may be used.

FIG. 13A is a graph depicting the zeta potential of surface modified paramagnetic silica beads. Parent beads and beads modified with only APTS or THPMP were used as standards.

FIG. 13B is a graph depicting bead fluorescence after reaction of each bead type shown with NHS ester fluorescein. Only beads modified with surface amines, and not acetylated, were highly fluorescent.

FIG. 14 is a chemical reaction schematic of CDAP activation of polysaccharides and immobilization on the surface of amine phosphonate beads. Although only a single polysaccharide is used in this depiction, any glycan may be used.

FIG. 15 is a graph depicting arabinoxylan immobilization on surface modified beads. Beads were reacted with CDAP-activated arabinoxylan in the presence of catalytic TEA. The amount of arabinoxylan bound to each bead type was determined by quantifying xylose and arabinose liberated following acid hydrolysis of a set number of beads.

FIG. 16 is a schematic of the use of polysaccharide-coated beads to measure the biochemical function of a gut microbiota within a mouse.

FIG. 17 is a graph depicting arabinose release from polysaccharide-coated beads harvested from cecum 4 hours post bead gavage. Each data point represents a single mouse. Mean±SD. Pairwise Welch's t-test. Benjamini and Hochberg corrected. *p<0.05.

FIG. 18 diagrams a procedure for fractionation of a pea fiber preparation.

FIG. 19 is graph depicting monosaccharide compositions of fractions 1 to 8 of a pea fiber preparation.

FIG. 20A depicts the structure of a pea fiber arabinan. R groups (not shown) are attached to each end, where R may be hydrogen or a pectic fragment. The proposed chemical structure for pea fiber arabinan is derived from partially methylated alditol acetate GC-MS analysis which was supported by the Chemical Sciences, Geosciences and Biosciences Division, Office of Basic Energy Sciences, U.S. Department of Energy grant (DE-SC0015662) to DOE—Center for Plant and Microbial Complex Carbohydrates at the Complex Carbohydrate Research Center. In Fraction 8, R₁ is H and R₂ is a pectic fragment containing galacturonic acid, galactose, and rhamnose.

FIG. 20B depicts the structure of a sugar beet arabinan. An R group (not shown) is attached to the free end, where R may be hydrogen or a pectic fragment.

FIG. 21 is graph depicting monosaccharide compositions of sugar beet arabinan and Fraction 8.

FIG. 22 is an illustration of the experimental design described in Example 10.

FIG. 23 is a graph of a principal component analysis of fecal bacterial community composition in response to diet supplementation. Each data point represents an individual mouse. Shaded regions represent 95% probability region of the s.d. of mean.

FIG. 24 graphically depicts the fractional abundance of several bacterial strains following diet supplementation. Each circle represents an individual mouse. Shaded regions are ±SD.

FIG. 25 is an illustration of the experimental design described in Example 10.

FIG. 26 are graphs depicting arabinose mass following diet supplementation.

FIG. 27A, FIG. 27B, and FIG. 27C are alignments of arabinan-utilization loci arabinan-utilization loci in Bacteroides species (related to FIG. 2). Alignment of B. thetaiotaomicron PUL7 (FIG. 27A), B. cellulosilyticus PUL5 (FIG. 27B), and B. vulgatus PUL27 (FIG. 27C) across multiple strains of each species. The direction of transcription is indicated by the arrowhead. The genes are labeled with their locus tag number and color-coded according to their functional annotation (see key). Shaded regions connecting genes denote (i) significant BLAST homology (E-value<10⁻⁹) and the percent amino acid identity of their protein products (see key).

FIG. 28A, FIG. 28B, and FIG. 28C are graphs showing B. cellulosilyticus-dependent glycan use by B. ovatus in the HiSF-LoFV diet context (related to FIG. 6). Proteomics analysis of fecal communities sampled on experimental days 6, 12, 19, and 25. Genes, color-coded according to their functional annotation including GH family assignments, in the indicated PULs are shown along the x-axis together with their locus tag numbers (Bovatus_0XXXX). The abundance of their expressed protein products (mean values±SD) is plotted along the y-axis (n=5 animals/treatment group). Key for circles: grey, 15-member community; magenta, mice harboring communities without B. cellulosilyticus. *, P<0.05, |fold change|>log 2(1.2), [15-member community versus 14-member (minus B. cellulosilyticus); limma].

FIG. 29 diagram a process for making a food composition (e.g., extruded pillow).

FIG. 30A shows an illustration of the study design of Example 12.

FIG. 30B show descriptions of singular value decomposition and higher-order singular value decomposition. The top part of the illustration shows a matrix M, defined by n rows and m columns, is analyzed by SVD to create three new matrices: U (dimensions n by n), E (dimensions n by m), and V (dimensions m by m). Columns of the U matrix are termed ‘Left Singular Vectors’ (LSVs), diagonal entries of E are termed “Singular Values”, rows of V are termed ‘Right Singular Vectors’ (RSVs). Multiplication of the first LSV (LSV1), the first singular value, and the first RSV (RSV1) creates a matrix, M¹, that reflects variation contained within the first singular value exclusively. The bottom part of the illustration shows a tensor, O, defined by n rows, m columns, and p conditions, is analyzed by HO-SVD to create a core tensor populated by diagonal elements only, G, and three matrices (dimensions n by a, m by b, and p by c). The dimensions of the core tensor G (a, b, and c) are determined from a numeric approximation method used in HO-SVD known as Canonical-Polyadic Alternating Least Squares (CP-ALS). The fractional variance captured by tensor component 1 (TC1) is reflected by the value of the first element of G (a₁, b₁, c₁) (red-shaded cube in core tensor G). The result of HO-SVD on O results in computing contributions, or ‘projections’ of each degree of freedom (n, m, or p) on each tensor component. The projections of each degree of freedom on TC1 are highlighted in red.

FIG. 30C, FIG. 30D, FIG. 30E, and FIG. 30F show the results of testing for the effects of dietary fibers in gnotobiotic mice colonized with nine different obese human donor microbiota and fed a HiSF-LoFV USA diet. FIG. 30C shows a plot of microbiome configurations on TC1 and TC2 as a function of diet treatment resulting from HO-SVD applied to CAZymes in fecal microbiomes of mice colonized with nine different obese human donor microbial communities during the pea fiber phase of the diet oscillation experiment. FIG. 30D shows a histogram of CAZyme projections on TC1 where the CAZyme genes that project within the most positive and negative 10^{th} percentiles are highlighted in red and yellow respectively. FIG. 30E and FIG. 30F show heatmaps of log₂ fold-change discriminatory CAZymes shown in FIG. 30D. Data are averaged for animals containing a given human donor microbiota sampled at the indicated time points and normalized to day 14 values. The depicted order of CAZymes ranked from top to bottom of the heatmap (beginning with FIG. 30E and ending with FIG. 30F) is based on their projections along TC1 in FIG. 30D, beginning with the most negatively projecting CAZyme (GH102 positioned at the top of the left-most column) and ending with the most positively projecting CAZymes (PL6 and GH99 positioned at the bottom of the right-most column).

FIG. 31A, FIG. 31B, FIG. 31C, FIG. 31D, FIG. 31E, FIG. 31F, FIG. 31G, FIG. 31H, FIG. 31I, FIG. 31J, and FIG. 31K show results of a controlled diet study of the effects of fiber-snack food prototypes on the fecal microbiomes of overweight and obese humans. FIG. 31A shows an illustration of the study design of Example 14. FIG. 31B shows an illustration of the study design of Example 15. FIG. 31C-E show HO-SVD analyses of changes in microbiome configurations as a function of fiber snack prototype, defined by the representation of discriminatory CAZymes where FIG. 31C was defined by the CAZyme pea fiber; FIG. 31D was defined by the CAZyme pea fiber and inulin; and FIG. 31E was defined by the CAZyme pea fiber, inulin, orange fiber and barley bran. FIG. 31F-K show heatmaps plotting the log₂ fold-change in the abundances of these discriminatory CAZymes relative to the time of initiation of pea fiber snack consumption (day 14) (FIG. 31F, FIG. 31G); initiation of pea fiber and inulin snack consumption (day 11) (FIG. 31H, FIG. 31I); and initiation of pea fiber, inulin, orange fiber and barley bran snack consumption (day 11) (FIG. 31J, FIG. 31K). In FIG. 31F-K, hierarchical clustering (Canberra distance) provided a way to operationally group participant microbiomes as responsive or hypo-responsive to the intervention (branches colored red and black, respectively, in the dendrograms shown).

FIG. 32A, FIG. 32B, FIG. 32C, FIG. 32D, FIG. 32E, and FIG. 32F show host responses defined by plasma proteomic features in a controlled diet study. FIG. 32A, FIG. 32C, and FIG. 32E show HO-SVD analyses of changes in the plasma proteomes of subjects in each of the indicated treatment groups sampled at the indicated time points where FIG. 32A is the group that consumed pea fiber snacks, FIG. 32C is the group that consumed pea fiber and inulin snacks, and FIG. 32E is the group that consumed pea fiber, inulin, orange fiber and barley bran snacks. FIG. 32B, FIG. 32D, and FIG. 32F show log₂ fold-changes in the abundances of 25 discriminatory plasma proteins assigned to the KEGG insulin and glucagon signaling pathways, in each subject as a function of the different snack fiber prototype treatments changes normalized to day 14 in the pea fiber study (FIG. 32B) and day 11 for the effects of the two- (FIG. 32D) and four-fiber (FIG. 32F) formulations. The direction of change in the abundance of each of these proteins that is indicative of movement towards a healthier state is denoted by the vertical bar on right side of the heatmaps (increase in red or a decrease in blue). Subjects were classified as responsive or hypo-responsive to the snack food prototype interventions based on an aggregate change of 50% of these 25 protein markers towards a healthier state plus the results of hierarchical clustering (Canberra distance).

FIG. 33A, FIG. 33B, FIG. 33C and FIG. 33D show cross correlation singular value decompositions (CC-SVD) relating host proteomic responses to changes in CAZyme gene representation in the microbiomes of subjects consuming the four-fiber snack prototype. FIG. 33A and FIG. 33B show a summary of the CC-SVD method where each element of the cross-correlation matrix contains the Spearman rank-correlation between CAZyme i and protein j (FIG. 33A). The singular value decomposition (SVD) of the cross-correlation matrix is shown in FIG. 33B. The left singular matrix contains projections of CAZymes along SV1 while the right singular matrix contains projections of proteins along SV1. Singular values, which relate the left and right SVs (and by extension, the CAZymes proteins with the plasma proteins) are housed in the central matrix. Histograms of projections of CAZymes (orange) and proteins (blue) along SV1. CAZymes and proteins within the same tail of the distribution are strongly positively correlated while those in opposite tails of the distribution are strongly negatively correlated. FIG. 33C-D show heatmaps plotting the Spearman correlation coefficient between CAZymes and proteins for the four-fiber, two-fiber, and pea-fiber alone snack prototypes. The blank column in FIG. 33C indicates that measurement of TFF2 in the plasma proteomes of study 1 participants consuming pea fiber did not pass quality control criteria. The coloring indicated in FIG. 33C for the enzymes/CAZyme designation applies to FIG. 33D. The key in FIG. 33D applies to FIG. 33C.

FIG. 34A, FIG. 34B, FIG. 34C, FIG. 34D and FIG. 34E show monosaccharide content (FIG. 34A, FIG. 34B, FIG. 34C, FIG. 34D) and glycosyl linkages (FIG. 34E) present in unsupplemented and fiber-supplemented HiSF-LoFV diets fed to gnotobiotic mice. Data shown are *p<0.01, ***, p<0.001 as determined by a one-way ANOVA with Holm-Sidak multiple comparison correction. Linkages shown are represented by their methylated monosaccharide derivatives Abbreviations. Glc, glucose; Gal, galactose; GalA, galacturonic acid; GlcA, glucuronic acid; Ara, arabinose; Xyl, xylose; Fru, fructose; Fuc, fucose; Rha, rhamnose; Rib, ribose; Hex, hexose; dHex, deoxyhexose; T, terminal; f, furanose; p, pyranose; X, undefined linkage.

FIG. 35A, FIG. 35B, FIG. 35C, FIG. 35D, FIG. 35E, FIG. 35F, FIG. 35G, FIG. 35H, and FIG. 35I show results of HO-SVD applied to ASV and mcSEED metabolic pathway datasets generated from the fecal microbiota of mice harboring nine different obese human donor microbial communities during the pea-fiber phase of the diet oscillation. FIG. 35A shows projections of microbiota configuration as defined by representation on TC1 and TC2. FIG. 35B shows a histogram of ASV projections on TC1; taxa that project within the most positive and negative 10^{th} percentiles are highlighted in red and yellow, respectively. FIG. 35C, FIG. 35D, FIG. 35CE show heatmaps of fractional abundances of a subset of the taxa highlighted in FIG. 35B where each column indicates the human donor microbiota used to colonize the mice. Data are averaged for all mice in a given treatment group on the indicated experimental days. FIG. 35F shows microbiome configurations as defined by the representation of mcSEED metabolic pathways. FIG. 35G shows a histogram that highlights pathways that project within the most positive and negative 10^{th} percentiles. FIG. 35H and FIG. 35I show heatmaps depicting the log₂ fold-change for representation of discriminatory mcSEED metabolic pathways identified in FIG. 35G. Data are averaged for all mice in the indicated treatment groups at the indicated time points and normalized to day 14 values.

FIG. 36A, FIG. 36B, FIG. 36C, FIG. 36D, FIG. 36E and FIG. 36F show results of HO-SVD applied to genes encoding CAZymes present in the fecal microbiomes of mice during the orange fiber treatment phase of their diet oscillation. FIG. 36A shows changes in microbiome configuration as defined by CAZyme gene abundances. FIG. 36B shows a histogram of CAZyme projections on TC1 and TC2 with those projecting within the most positive and negative 10^{th} percentiles highlighted in red and yellow. FIG. 36C, FIG. 36D, FIG. 36E and FIG. 36F show a heatmaps of log₂ fold-change for the discriminatory CAZymes shown in FIG. 36B. Data are averaged for all mice in the indicated treatment groups at the indicated time points and normalized to day 14 values. FIG. 36C and FIG. 36D are day 44; FIG. 36E and FIG. 36F are day 54.

FIG. 37A, FIG. 37B, FIG. 37C, FIG. 37D, FIG. 37E, FIG. 37F, FIG. 37G, and FIG. 37H show results of HO-SVD applied to the ASV and mcSEED pathway datasets generated from mice during the orange fiber phase of the diet oscillation. FIG. 37A shows projections of microbiota configuration as defined by representation on TC1 and TC2. FIG. 37B shows a histogram of ASV projections on TC1; taxa that project within the most positive and negative 10^{th} percentiles are highlighted in red and yellow, respectively. FIG. 37C, FIG. 37D, FIG. 37E show heatmaps of fractional abundances of a subset of the taxa highlighted in FIG. 37B. Each column indicates the human donor microbiota used to colonize the mice. Data are averaged for all mice in a given treatment group on the indicated experimental days. FIG. 37F shows microbiome configurations as defined by the representation of mcSEED metabolic pathways. FIG. 37G shows a histogram that highlights pathways that project within the most positive and negative 10^{th} percentiles. FIG. 37H shows a heatmap depicting the log₂ fold-change in the representation of discriminatory mcSEED metabolic pathways identified in FIG. 37G. Data are averaged for all mice in the indicated treatment groups at the indicated time points and normalized to day 14 values.

FIG. 38A, FIG. 38B, FIG. 38C, FIG. 38D, FIG. 38E, and FIG. 38F show results of HO-SVD applied to CAZymes genes represented in the fecal microbiomes of mice colonized with the obese human donor microbial communities during the barley bran fiber phase of the diet oscillation. FIG. 38A shows changes in microbiome configuration as defined by CAZyme gene abundances. FIG. 38B shows a histogram of CAZyme projections on TC1 and TC2 with those projecting within the most positive and negative 10^{th} percentiles highlighted in red and yellow, respectively. FIG. 38C, FIG. 38D, FIG. 38E and FIG. 38F show heatmaps of log₂ fold-change for the discriminatory CAZymes shown in FIG. 38B at day 54 (FIG. 38C and FIG. 38D) and day 65 (FIG. 38E and FIG. 38F). Data are averaged for all mice in the indicated treatment groups at the indicated time points and normalized to day 14 values.

FIG. 39A, FIG. 39B, FIG. 39C, FIG. 39D, FIG. 39E, FIG. 39F, and FIG. 39G show results of HO-SVD of ASV and mcSEED pathway representation in the fecal communities of mice during the barley bran fiber phase of the diet oscillation. FIG. 39A shows projections of microbiota configuration as defined by representation on TC1 and TC2. FIG. 39B shows a histogram ASV projections on TC1; taxa that project within the most positive and negative 10^{th} percentiles are highlighted in red and yellow, respectively. FIG. 39C and FIG. 39D show heatmaps of fractional abundances of a subset of the taxa highlighted in FIG. 39B. Each column indicates the human donor microbiota used to colonize the mice. Data are averaged for all mice in a given treatment group on the indicated experimental days. FIG. 39E shows microbiome configurations as defined by the representation of mcSEED metabolic pathways. FIG. 39F shows a histogram that highlights pathways positioned within the most positive 10^{th} percentile and most negative 20^{th} percentile of projections along TC1. FIG. 39G shows a heatmap depicting the log₂ fold-change in representation of discriminatory mcSEED metabolic pathways identified in FIG. 39F. Data are averaged for all mice in the indicated treatment groups at the indicated time points and normalized to day 14 values.

FIG. 40A, FIG. 40B, and FIG. 40C show CAZymes identified by HO-SVD analysis as discriminatory for microbiome responses to the different fiber snack food prototypes. FIG. 40A, FIG. 40B, and FIG. 40C show histograms of CAZyme projections on the indicated tensor components for pea fiber snack food (FIG. 40A) and the two- (FIG. 40B) and four-fiber (FIG. 40C) snack food formulations. CAZyme genes that project within the most positive and negative 20^{th} percentiles are highlighted in red and yellow, respectively. The dashed box relates the rank order of CAZymes from top to bottom of the heatmaps shown in FIG. 31F-K to their projections along the tensor components described in these histograms, beginning with the most positively projecting CAZyme at the bottom of the right-most column and proceeding to the upper most CAZyme in the first column encompassed within the box (i.e., for pea fiber, the most positive projecting CAZyme, CMB77, is located at the top of the heatmap displayed in FIG. 31F,G while CBM3 is positioned at the bottom of the heatmap; for the two-fiber formulation, PL29 is located at the top and GH89 at the bottom of the heatmap in FIG. 31H,I; for the four-fiber formulation, PL38 is at the top and CMB77 is at the bottom of the heatmap in FIG. 31J,K).

FIG. 41A, FIG. 41B, FIG. 41C, FIG. 41D, FIG. 41E, FIG. 41F, FIG. 41G, FIG. 41H, and FIG. 41I show results of HO-SVD analysis of the effects of the pea fiber snack prototype on representation of pea fiber treatment-discriminatory mcSEED metabolic pathways and ASV taxa present in the fecal microbiomes of subjects enrolled in human study 1. FIG. 41A shows projections of microbiome configuration of mcSEED metabolic pathways on TC1 and TC3. FIG. 41B shows a histogram of mcSEED metabolic pathways projections on TC3; metabolic pathways that project within the most positive and negative 20^{th} percentiles are highlighted in red and yellow, respectively. FIG. 41C shows a heatmap of the log₂ fold-change at day 29 (consumption of the maximum dose of the pea fiber snack prototype) in the representation of discriminatory mcSEED metabolic pathways identified in FIG. 41B, normalized to day 14 (last pre-treatment timepoint). Each column indicates a subject. FIG. 41D shows microbiota configurations. FIG. 41E shows a histogram that highlights ASVs that project within the most positive and negative 20^{th} percentiles on TC2. FIG. 41F, FIG. 41G, FIG. 41H, and FIG. 41I show heatmaps depicting the fractional abundances of ASVs identified in FIG. 41E for days 14 (last day of pre-treatment, FIG. 41F and FIG. 41G), and day 29 (consumption of the maximum dose of the pea fiber snack prototype, FIG. 41H and FIG. 41I). Each row indicates a participant, the rows are identified in FIG. 41F and FIG. 41H, and the identifiers also apply to FIG. 41G and FIG. 41I.

FIG. 42A and FIG. 42B show spearman-rank cross-correlation analyses of representation of CAZymes by monosaccharides and glycosyl linkages in the fecal communities of subjects consuming the pea fiber snack prototype. Correlations between the log₂ fold-change of HO-SVD defined discriminatory CAZyme gene abundances (matched by time and subject) to the log₂ fold-change in levels of monosaccharides (FIG. 42A) and glycosidic linkages (FIG. 42B) normalized to day 14 (pre-intervention phase). Monosaccharides abundant in pea fiber that are significantly positively correlated with discriminatory CAZymes which increased during pea fiber supplementation are highlighted by the green boxes in FIG. 42A. Each row of the heatmap is a monosaccharide. From top to bottom, the rows are Rib, Xyl, GalA, Ara, Fru, Fuc, Glc, Man, GlcA, Gal, All, Rha, GlcNAc, GalNAc. Each column is a CAZyme. From left to right, the columns are: GH43_4, PL27, GH43_37, PL11, GH115, GH43_19, GT101, GH43_29, CBM6, CBM27, CBM23, GH43_5, GH10, GH82, CBM61, CBM22, CBM4, GH5_2, CBM72, GT17, GT76, GH30_5, GH97, PL8, GH43_2, PL6, GH5_5, GH50, PL17, PL15, PL13, PL12, GH5_21, GH43_1, GH67, GH108, GH5_1, GH30_8, GH43_7, CBM37, CBM2, GH30, PL30, GH26, PL1, PL9, CBM77, GH13_8, GT3, GH19, GH13_38, GT30, GH5_7, GH30_3, GH57, GH9, GH5_8, GH5_4, GH44, CBM3, CBM79, CBM78. FIG. 42B provides evidence that subject microbiomes contain CAZymes that cleave multiple branches of pea fiber arabinan, resulting in accumulation of its 1,5-arabinofuranose backbone in feces. Each row indicates a glycosidic linkage. From top to bottom, the rows are: 5-Ara(f); 2-Xyl; X,X-Hex (I); 2-Gal; 4-Man/3-Man; 4-Glc; 4,6-Glc/3,6-Gal; X,X,X-Hex (I); 3-Xyl; 2,X1-Xyl; 2,X-Hex (I); 2,X,X-Hex (I); 4-Xyl(p); 2,X,X-Hex (II); 2,X2-Ara; 2-Ara(f); 3-Ara(f); 3,4,6-Man, 3,4,6-Gal; 3,6-Man; 6-Glc/6-Gal; 4-Gal/6-Man; 2,X-dHex (III); T-Man; 3-Glc/3-Gal; T-Gal; T-Rha; 2,X-dHex (II); T-Ara(f); T-Glc; T-Fuc; X-Hex; 2-Man; 2-Glc; 4,6-Man; 3,4-Xyl(p); Xyl(p); 2,X1-Ara; 2,X2-Rib; 2,X-Hex (II); 2-dHex (I); X-dHex (I); 3,4-Fuc; X-dHex (II); T-fru; 2,X-dHex (I). Each column is a CAZyme. From left to right, the columns are: GH5_2, GH5_7, CBM4, GH5_21, GH67, GH10, GH57, GH97, GT19, GH13_38, GH13_8, GT3, GT30, GH43_2, GT101, GH43_29, PL1, CBM6, GH82, PL11, GH43_1, GH43_19, GH115, GH43_4, GH43_37, GT76, CBM27, CBM77, GH43_7, GH5_8, CMB2, CMB79, GH44, GH5_1, CMB78, CMB3, GH5_37, GH30_8, GH26, GH5_4, GH108, GH30_3, GT17, PL17, CBM61, PL27, GH30, GH9, PL30, PL6, GH5_5, PL8, CBM23, CBM22, PL9, GH50, PL12, PL15, PL13, GH43_5, CBM72, CBM37, GH30_5. Abbreviations: glucose (Glc), galacturonic acid (GalA), arabinose (Ara), xylose (Xyl), galactose (Gal), mannose (Man), rhamnose (Rha), fucose (Fuc), fructose (Fru), glucuronic acid (GlcA), N-acetylglucosamine (GlcNAc), N-acetylgalactosamine (GaINAc), allose (All), ribose (Rib), hexose (Hex), deoxyhexose (dHex), terminal (T), pyranose (p), furanose (f), undefined linkage (X).

FIG. 43A, FIG. 43B, FIG. 43C, FIG. 43D, FIG. 43E, FIG. 43F, and FIG. 43G show HO-SVD analysis of the effects of the two-fiber snack prototype on the representation of treatment discriminatory mcSEED metabolic pathways and ASVs in the fecal communities of subjects enrolled in human study 2. FIG. 43A shows projections of microbiome configuration based on mcSEED metabolic pathway composition. FIG. 43B shows a histogram of mcSEED metabolic pathways projections on TC2; pathways that project within the most positive and negative 20^{th} percentiles are highlighted in red and yellow, respectively. FIG. 43C shows a heatmap of the log₂ fold-change at day 25 (maximum dose of the two-fiber snack prototype) in the representation of discriminatory mcSEED metabolic pathways identified in FIG. 43B, normalized to day 11 (last day of pre-treatment). Each column indicates a subject. FIG. 43D shows microbiota configurations based on ASV composition. FIG. 43E shows a histogram that highlights taxa that project within the most positive and negative 20th percentiles on TC2. FIG. 43F, FIG. 43G, FIG. 43H, and FIG. 43I show heatmaps depicting the fractional abundances of discriminatory ASVs identified in FIG. 43E for days 11 (last day of pre-treatment, FIG. 43F and FIG. 43G), and day 25 (consumption of the maximum dose of the two-fiber snack prototype, FIG. 43H and FIG. 43I). Each row indicates a participant. The rows are identified in FIG. 43F and FIG. 43H, and the identifiers also apply to FIG. 43G and FIG. 43I.

FIG. 44A, FIG. 44B, FIG. 44C, FIG. 44D, FIG. 44E, FIG. 44F, FIG. 44G, FIG. 44H, and FIG. 44I show HO-SVD analysis of the effects of the four-fiber snack prototype on the representation of treatment discriminatory mcSEED metabolic pathways and ASVs in the fecal communities of subjects enrolled in human study 2. FIG. 44A shows projections of microbiome configuration based on mcSEED metabolic pathways composition. FIG. 44B shows a histogram of mcSEED metabolic pathways projections on TC1; metabolic pathways that project within the most positive and negative 20^{th} percentiles are highlighted in red and yellow, respectively. FIG. 44C shows a heatmap of the log₂ fold-change at day 49 (consumption of the maximum dose of the four-fiber snack prototype) in the representation of discriminatory mcSEED metabolic pathways identified in FIG. 44B, normalized to day 11 (last day of pre-treatment). Each column indicates a subject. FIG. 44D shows microbiota configurations as defined by the representation of ASVs. FIG. 44E shows a histogram that highlights taxa that project within the most positive and negative 20^{th} percentiles on TC1. FIG. 44F, FIG. 44G, FIG. 44H, and FIG. 44I show heatmaps depicting the fractional abundances of discriminatory ASVs, identified in FIG. 44E, for days 35 (last day of washout-phase; FIG. 44E and FIG. 44F), and day 49 (consumption of the maximum dose of the four-fiber snack prototype; FIG. 44G, and FIG. 44H). Each row indicates a participant. The rows are identified in FIG. 44F and FIG. 44H, and the identifiers also apply to FIG. 44G and FIG. 44I.

FIG. 45A, FIG. 45B, and FIG. 45C show LC-QTOF-MS analysis of a biomarker of orange fiber consumption present in gnotobiotic mouse and human fecal samples. FIG. 45A shows a comparison of levels of the m/z 274.1442 analyte in colonized and germ-free mice fed the unsupplemented, orange fiber-supplemented or pea fiber-supplemented HiSF-LoFV diet for 10 days. The analyte is only detectable when orange fiber is consumed and is not dependent upon on the donor microbiome for its generation. FIG. 45B and FIG. 45C show comparisons of levels of the analyte in fecal samples obtained from participants in human study 2 on days 25 and 49 when they were consuming the maximum dose of the two-fiber (pea and inulin) and four-fiber (pea fiber, inulin, orange fiber plus barley bran) snack food prototypes where FIG. 45B shows the average analyte amount and FIG. 45C shows the analyte amount in the fecal samples of each individual. The horizontal dashed line in FIG. 45C denotes a baseline value operationally defined as the highest level of detection of the analyte in subjects consuming the two-fiber snack food prototype lacking orange fiber.

### DETAILED DESCRIPTION

The present disclosure provides compositions and foods that selectively promote the representation and expressed beneficial function of members of a human gut community in ways that promote a healthy gut microbiota (e.g., improve fiber degrading capacity) and in turn positively impact health. The effects of the fiber supplements on gut microbial community configuration (representation of microbial taxa, genes encoding carbohydrate-active enzymes and genes encoding proteins and enzymes in various metabolic pathways), gut microbial function (activity of genes encoding carbohydrate-active enzymes and/or genes encoding proteins and enzymes in various metabolic pathways) and host biology (which may be defined by changes in the levels of plasma proteins representing biomarkers and mediators of numerous physiologic, metabolic, and immune functions) are shown to be specific. Therefore, these specific effects can be considered discriminatory features and can be used to provide a rigorous scientific foundation for claims about the benefits of these products for different consumers with different weights, body mass indices, diets, and health. For instance, responders may be defined as those subjects with an aggregate change of 50% towards a healthier state for a collection of plasma protein markers (e.g., protein markers of chronic inflammation, protein markers of insulin and/or glucagon signaling, protein markers of satiety, protein markers of weight management, protein markers of cardiovascular health, etc.). The collection of plasma protein biomarkers in the Examples are defined by the proteomic assay (e.g., SOMAscan Assay 1.3k) but other assays can be used. Alternatively, or in addition, responders may be defined as those subjects with an aggregate change of ≤50% towards a healthier state in the representation of health discriminatory CAZymes, mcSEED subsystem proteins, or microbial taxa. Various examples of compositions and foods of the present disclosure, which comprise one or more fiber preparation, are discussed in detail below, as are methods of their use. In addition, Applicants have identified bioactive components in compositionally complex food ingredients that increase the fiber degrading capacity of the gut microbiota.

While specific implementations are discussed, it should be understood that this is done for illustration purposes only. A person skilled in the relevant art will recognize that other components and configurations may be used without parting from the spirit and scope of the disclosure. Additional features and advantages of the disclosure will be set forth in the description which follows, and in part will be obvious from the description, or can be learned by practice of the herein disclosed principles. These and other features of the disclosure will become more fully apparent from the following description and appended claims, or can be learned by the practice of the principles set forth herein.

Several definitions that apply throughout this disclosure will now be presented.

As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result. In some instances, the term “about” may include numerical values that are rounded to the nearest significant figure.

The term “comprising” means “including, but not necessarily limited to”; it specifically indicates open-ended inclusion or membership in a so-described combination, group, series and the like. The terms “comprising” and “including” as used herein are inclusive and/or open-ended and do not exclude additional, unrecited elements or method processes.

As used herein, the term “fiber preparation” refers to a composition comprising dietary fiber that (i) is intended as an ingredient in a food, and (ii) has been prepared from a plant source including, but not limited to, fruits, vegetables, legumes, oilseeds, and cereals; or has been otherwise manufactured to have a composition similar to a fiber preparation prepared from a plant source. “Prepared from a plant source,” as used herein, indicates plant material has undergone one or more treatment step prior to its utilization to make a composition disclosed herein (e.g., grinding, milling, shelling, hulling, extraction, extrusion, fractionation, etc.).

The term “dietary fiber” refers to edible parts of plants, or analogous glycans and carbohydrates, that are resistant to digestion and adsorption in the human small intestine with complete or partial fermentation in the large intestine. The term “dietary fiber” includes glycans, lignin, and associated plant substances. Total dietary fiber, soluble dietary fiber, and insoluble dietary fiber are terms of art defined by the methodology used to measure their relative amount. As used herein, total dietary fiber is defined by AOAC method 2009.01; soluble dietary fiber and insoluble dietary fiber are defined by AOAC method 2011.25.

The term “carbohydrate” refers to an organic compound with the formula Cₘ(H₂O)ₙ, where m and n may be the same or different number, provided the number is greater than 3.

As used herein, the term “glycan” refers to a homo- or heteropolymer of two or more monosaccharides linked glycosidically. As such, the term “glycan” includes disaccharides, oligosaccharides and polysaccharides. The term also encompasses a polymer that has been modified, whether naturally or otherwise; non-limiting examples of such modifications include acetylation, alkylation, esterification, etherification, oxidation, phosphorylation, selenization, sulfonation, or any other manipulation. Glycans may be linear or branched, may be produced synthetically or obtained from a natural source, and may or may not be purified or processed prior to use.

A glycan may be defined, in part, in terms of its monosaccharide content and its glycosyl linkages. For example, plant arabinans are composed of 1,5-α-linked L-arabinofuranosyl residues, and these can be branched at O-2 or O-3 by single arabinosyl residues or short side chains (Beldman et al., 1997; Ridley et al., 2001; Mohnen, 2008). 1,5-Linked arabinan structures exist as free polymers unattached to pectic domains or attached to pectic domains (Beldman et al., 1997; Ridley et al., 2001).

As is understood in the art, due to the mechanism of side chain synthesis, a plant glycan is not a single chemical entity but is rather a mixture of glycans that have a defined backbone and variable amounts of substituents/branching. It is routine in the art to indicate the presence of variable amounts of a substituent by indicating its fractional abundance. For instance, when R₁ and R₂ are each H, the glycan depicted below is an arabinan—specifically, a polymer consisting of 1,5-α-linked L-arabinofuranosyl residues:

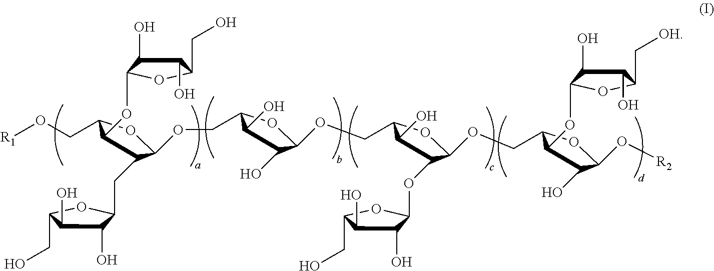

The formula indicates that (1) the polymer backbone consists of 1,5-α-linked L-arabinofuranosyl residues, and (2) there are 4 types of arabinose components—namely, component a—2,3,5-arabinofuranose, component b—5-arabinofuranose, component c—2,5-arabinofuranose, and component d—3,5-arabinofuranose. The fractional abundance of each component is indicated by the values assigned to a, b, c, and d, respectively. The sum of all the values is about 1 (allowing for a small amount of error in the measurements). A value of zero (0) indicates the component is never present in the polymer. A value of one (1) indicates the component accounts for 100% of the polymer. A value of 0.5 indicates that the component accounts for 50% of the polymer. The arrangement of the components within the polymer can vary, as is understood in the art, and is not defined by the order depicted.

The term “compositional glycan equivalent” refers to a fiber preparation with a substantially similar glycan content as the composition to which it is being compared. A compositional glycan equivalent may be substituted about 1:1 for its comparison composition because the compositional glycan equivalent has a glycan content similar to the composition it is replacing. For instance, if about 30 wt % of pea fiber preparation is to be replaced with a compositional glycan equivalent thereof, one of skill in the art would use about 30 wt % of the pea fiber glycan equivalent. A compositional glycan equivalent may be defined in terms of its monosaccharide content and optionally by an analysis of the glycosidic linkages. Methods for measuring monosaccharide content and analyzing glycosidic linkages are known in the art, and described herein.

The term “functional glycan equivalent” refers to a fiber preparation with substantially similar function as the composition to which it is being compared. The amount of a functional glycan equivalent needed to achieve a substantially similar function may be about the same as the comparison composition, or may be less. For instance, a compositional glycan equivalent will typically have substantially similar function as its comparison composition on a 1:1 (weight) basis. However, an enriched bioactive fraction of a composition may have substantially similar function as the initial composition, but comprise less material, and therefore, less weight than the initial composition. The present disclosure contemplates these and other functional glycan equivalents, as illustrated in Example 10. Substantially similar function may be measured by any method detailed in the Examples herein, in particular the ability to affect total abundance(s) of microbial community members, relative abundance(s) of microbial community members, expression of microbial genes, abundance of microbial gene products (e.g. proteins), activity of microbial proteins, and/or observed biological function of a microbial community.

A “food” or a “food composition” is an article to be taken by mouth. The form of the food or food composition can vary, and includes but is not limited to a powder form which may be reconstituted or sprinkled on a different food; a bar; a drink; a gel, a gummy, a candy, or the like; a cookie, a cracker, a cake, or the like; and a dairy product (e.g., yogurt, ice cream or the like). The term also encompasses a pill, capsule, tablet, or liquid. A “microbiota-directed food,” as used herein, refers to a food that selectively promotes the representation and/or expressed beneficial functions of targeted human gut microbes.

The term “microbiota” refers to microorganisms that are found within a specific environment, and the term “microbiome” refers to a collection of genes in the genomes of all the microorganisms found in a particular environment. Accordingly, the term “gut microbiota” refers to microorganisms that are found within a gastrointestinal tract of a subject, and a “gut microbiome” refers to a collection of genomes from all the microorganisms found in the gastrointestinal tract of a subject.

The “health” of a subject's gut microbiota may be defined by its features, namely its compositional state and/or its functional state. The “compositional state” of a gut microbiota refers to the presence, absence or abundance (relative or absolute) of microbial community members. The community members can be described by different methods of classification typically based on 16S rRNA sequences, including but not limited to operational taxonomic units (OTUs) and amplicon sequence variants (ASVs). The “functional state” of a gut microbiota refers to expression of microbial genes, observed biological functions, and/or phenotypic states of the community. A subject with an unhealthy gut microbiota has a measure of at least one feature of the gut microbiota or microbiome that deviates by 1.5 standard deviation or more (e.g., 2 std. deviation, 2.5 std. deviation, 3 std. deviation, etc.) from that of healthy subjects with similar environmental exposures, such as geography, diet, and age. To “promote a healthy gut microbiota in a subject” means to change the feature of the microbiota or microbiome of the subject with the unhealthy gut microbiota in a manner towards the healthy subjects, and encompasses complete repair (i.e., the measure of gut microbiota health does not deviate by 1.5 standard deviation or more) and levels of repair that are less than complete. Promoting a healthy gut microbiota in a subject also includes preventing the development of an unhealthy gut microbiota in a subject.

The “fiber degrading capacity” of a subject's gut microbiota may be defined by its compositional state and/or its functional state. For instance, the compositional stage of a subject's gut microbiota may be defined by the absence, presence and abundance of primary and secondary consumers of dietary fiber, while the functional state may be defined by the representation of relevant genomic loci (polysaccharide utilization loci (PULs), carbohydrate-active enzymes (CAZymes), etc.), expression from these loci, and/or activity of proteins encoded by these loci. An increase in the fiber degrading capacity of a subject may be effected by increasing the abundance of microorganisms with genomic loci for import and metabolism of glycans, as exemplified by PULs and/or loci encoding CAZymes; and/or increasing the abundance or expression of one or more proteins encoded by a PUL and/or one or more CAZyme (with or without concomitant changes in microorganism abundance).

As used herein, “statistically significant” is a p-value<0.05, or a comparable value calculated by other suitable methods.

The term “substantially similar” generally refers to a range of numerical values, for instance, ±0.5-1%, ±1-5% or ±5-10% of the recited value, that one would consider equivalent to the recited value, for example, having the same function or result.

The terms “relative abundance” and “fractional abundance” as used herein describe an amount of one or more microorganism. Relative abundance means the percent composition of a microorganism of a particular kind relative to the total number of microorganisms in the area. Fractional abundance is the relative abundance divided by 100. For example, the “relative abundance of Bacteroides in a subject's gut microbiota” is the percent of all Bacteroides species relative to the total number of bacteria constituting the subject's gut microbiota, as measured in a suitable sample. “Total abundance” refers to the total number of microorganisms. Suitable samples for quantifying gut microbiota include a fecal sample, a cecal sample or other sample of the lumen. A variety of methods are known in the art for quantifying gut microbiota. For example, a fecal sample, a cecal sample or other sample of the lumenal contents of the large intestine may be collected, processed, plated on appropriate growth media, cultured under suitable conditions (i.e., temperature, presence or absence of oxygen and carbon dioxide, agitation, etc.), and colony forming units may be determined. Alternatively, sequencing methods or arrays may be used to determine abundance. The examples detail one method, COPRO-Seq, where relative abundance is defined by the number of sequencing reads that can be unambiguously assigned to the species' genome after adjusting for genome uniqueness. 16S rRNA gene sequencing methods can also be used and are well known in the art.

These and other aspects of the present disclosure are detailed further below.

### I. Compositions of Fiber Preparations (Fiber Blends)

In one aspect, the present disclosure provides compositions comprising a plurality of fiber preparations. Compositions of this section may also be referred to herein as “a fiber blend.” Each fiber preparation can be independently selected from the group consisting of a barley fiber preparation, a citrus fiber preparation, a citrus pectin preparation, a high molecular weight inulin preparation, a pea fiber preparation, a sugar beet fiber preparation, and glycan equivalents thereof, wherein the plurality of fiber preparations is at least 95 wt %, at least 97 wt %, or at least 99 wt % of the composition. The present disclosure also provides compositions consisting essentially of a plurality of fiber preparations, each fiber preparation independently selected from the group consisting of a barley fiber preparation, a citrus fiber preparation, a citrus pectin preparation, a high molecular weight inulin preparation, a pea fiber preparation, a sugar beet fiber preparation and glycan equivalents thereof, wherein the plurality of fiber preparations is at least 95 wt %, at least 97 wt %, or at least 99 wt %, of the composition, and the remaining weight percent (if any) of the composition is comprised of one or more additional food ingredient that lacks dietary fibers. The amount of the plurality of fiber preparations in a composition may also be expressed as a range, for instance about 95 wt % to about 97 wt %, about 97 wt % to about 100 wt %, or about 98 wt % to about 100 wt %, etc.; or as individual values, for instance, 95 wt %, 96 wt %, 97 wt %, 98 wt %, 99 wt %, or 100 wt %. The glycan equivalent may be a functional glycan equivalent or a compositional glycan equivalent. The plurality of fiber preparations may be 2, 3, 4, 5, 6, 7, 8, 9, 10 or more different fiber preparations selected from the group consisting of a barley fiber preparation or a glycan equivalent thereof, a citrus fiber preparation or a glycan equivalent thereof, citrus pectin or a glycan equivalent thereof, a high molecular weight inulin preparation or a glycan equivalent thereof, a pea fiber preparation or a glycan equivalent thereof, and a sugar beet fiber preparation or a glycan equivalent thereof. In some embodiments, a composition may contain 2 or more different barley fiber preparations, 2 or more different citrus fiber preparations, etc. Various embodiments are described in further detail below.

In another aspect, the present disclosure provides compositions comprising at least 15 wt % of one or more pea fiber preparation or a glycan equivalent thereof; and at least one additional fiber preparation chosen from (i) at least 28 wt % of one or more high molecular weight inulin preparation or a glycan equivalent thereof, (ii) between 0 wt % and 10 wt % (inclusive) of one or more citrus pectin preparation or a glycan equivalent thereof, (iii) between 0 wt % and 25 wt % (inclusive) of one or more citrus fiber preparation or a glycan equivalent thereof, or (iv) between 0 wt % and 45 wt % (inclusive) of one or more barley fiber preparation or a glycan equivalent thereof. The composition may contain 2, 3, 4, 5, 6, 7, 8, 9, 10 or more different fiber preparations. Various embodiments are described in further detail below.

In some embodiments, a composition comprises (a) at least 15 wt % of one or more pea fiber preparation and/or at least 15 wt % of one or more sugar beet fiber preparation, and (b) at least 28 wt % of one or more high molecular weight inulin preparation, an amount of one or more citrus pectin preparation that does not exceed 10 wt %, an amount of one or more citrus fiber preparation that does not exceed 25 wt %, an amount of one or more barley fiber preparation that does not exceed 45 wt %, and no sugar beet fiber preparations. In another embodiment, a composition consists essentially of (a) at least 15 wt % of one or more pea fiber preparation and/or at least 15 wt % of one or more sugar beet fiber preparation, and (b) at least 28 wt % of one or more high molecular weight inulin preparation, an amount of one or more citrus pectin preparation that does not exceed 10 wt %, an amount of one or more citrus fiber preparation that does not exceed 25 wt %, an amount of one or more barley fiber preparation that does not exceed 45 wt %, and no sugar beet fiber preparations. In some embodiments, the citrus pectin preparation(s) is less than 1 wt %, or citrus pectin is absent from the composition. In further embodiments, the one or more citrus fiber is in an amount that does not exceed 15 wt %, or in an amount that does not exceed 12 wt %. In still further embodiments, the one or more barley fiber preparation is in an amount that does not exceed 30 wt %, or in an amount that does not exceed 20 wt %. In still further embodiments, there is either one or more pea fiber preparation or one or more sugar beet fiber preparation.

In some embodiments, a composition comprises (a) at least 28 wt % of one or more pea fiber preparation and/or at least 15 wt % of one or more sugar beet fiber preparation, and (b) at least 28 wt % of one or more high molecular weight inulin preparation, an amount of one or more citrus pectin preparation that does not exceed 10 wt %, an amount of one or more citrus fiber that does not exceed 25 wt %, an amount of one or more barley fiber preparation that does not exceed 45 wt %, and no sugar beet fiber preparation. In another embodiment, a composition consists essentially of (a) at least 28 wt % of one or more pea fiber preparation and/or at least 15 wt % of one or more sugar beet fiber preparation, and (b) at least 28 wt % of one or more high molecular weight inulin preparation, an amount of one or more citrus pectin preparation that does not exceed 10 wt %, an amount of one or more citrus fiber preparation that does not exceed 25 wt %, an amount of one or more barley fiber preparation that does not exceed 45 wt %, and no sugar beet fiber preparation. In some embodiments, the citrus pectin preparation(s) is less than 1 wt %, or citrus pectin is absent from the composition. In further embodiments, the one or more citrus fiber preparation is in an amount that does not exceed 15 wt %, or in an amount that does not exceed 12 wt %. In still further embodiments, the one or more barley fiber preparation is in an amount that does not exceed 30 wt %, or in an amount that does not exceed 20 wt %. In still further embodiments, there is either one or more pea fiber preparation or one or more sugar beet fiber preparation.

In some embodiments, a composition comprises (a) at least 30 wt % of one or more pea fiber preparation and/or at least 15 wt % of one or more sugar beet fiber preparation, and (b) at least 30 wt % of one or more high molecular weight inulin preparation, an amount of one or more citrus pectin preparation that does not exceed 10 wt %, an amount of one or more citrus fiber preparation that does not exceed 25 wt %, an amount of one or more barley fiber preparation that does not exceed 45 wt %, and no sugar beet fiber preparation. In another embodiment, a composition consists essentially of (a) at least 15 wt % of one or more pea fiber preparation and/or at least 15 wt % of one or more sugar beet fiber preparation, and (b) at least 28 wt % of one or more high molecular weight inulin preparation, an amount of one or more citrus pectin preparation that does not exceed 10 wt %, an amount of one or more citrus fiber preparation that does not exceed 25 wt %, an amount of one or more barley fiber preparation that does not exceed 45 wt %, and is no sugar beet fiber preparation(s). In some embodiments, the citrus pectin preparation(s) is less than 1 wt %, or citrus pectin is absent from the composition. In further embodiments, the one or more citrus fiber preparation is in an amount that does not exceed 15 wt %, or in an amount that does not exceed 12 wt %. In still further embodiments, the one or more barley fiber preparation is in an amount that does not exceed 30 wt %, or in an amount that does not exceed 20 wt %. In still further embodiments, there is either one or more pea fiber preparation or one or more sugar beet fiber preparation.

In some embodiments, a composition comprises (a) at least 35 wt % of one or more pea fiber preparation and/or at least 15 wt % of one or more sugar beet fiber preparation, and (b) at least 35 wt % of one or more high molecular weight inulin preparation, an amount of one or more citrus pectin preparation that does not exceed 10 wt %, an amount of one or more citrus fiber preparation that does not exceed 25 wt %, an amount of one or more barley fiber preparation that does not exceed 45 wt %, and no sugar beet fiber preparations. In another embodiment, a composition consists essentially of (a) at least 15 wt % of one or more pea fiber and/or at least 15 wt % of one or more sugar beet fiber preparation, and (b) at least 28 wt % of one or more high molecular weight inulin preparation, an amount of one or more citrus pectin preparation that does not exceed 10 wt %, an amount of one or more citrus fiber preparation that does not exceed 25 wt %, an amount of one or more barley fiber preparation that does not exceed 45 wt %, and no sugar beet fiber preparations. In some embodiments, the citrus pectin preparation(s) is less than 1 wt %, or citrus pectin is absent from the composition. In further embodiments, the one or more citrus fiber preparation is in an amount that does not exceed 15 wt %, or in an amount that does not exceed 12 wt %. In still further embodiments, the one or more barley fiber preparation is in an amount that does not exceed 30 wt %, or in an amount that does not exceed 20 wt %. In still further embodiments, there is either one or more pea fiber preparation or one or more sugar beet fiber preparation.

In some embodiments, a composition comprises (a) at least 15 wt % of one or more pea fiber preparation, at least 15 wt % of one or more sugar beet fiber preparation, or a glycan equivalent thereof, and (b) at least one additional fiber preparation chosen from: at least 28 wt % of one or more high molecular weight inulin preparation or a glycan equivalent thereof, 10 wt % of less of one or more citrus pectin preparation or a glycan equivalent thereof, 25 wt % or less of one or more citrus fiber preparation or a glycan equivalent thereof, and 45 wt % or less of one or more barley fiber preparation or a glycan equivalent thereof. In another embodiment, a composition consists essentially of (a) at least 15 wt % of one or more pea fiber preparation, at least 15 wt % of one or more sugar beet fiber preparation, or a glycan equivalent thereof, and (b) at least one additional fiber preparation chosen from: at least 28 wt % of one or more high molecular weight inulin preparation or a glycan equivalent thereof, 10 wt % of less of one or more citrus pectin preparation or a glycan equivalent thereof, 25 wt % or less of one or more citrus fiber preparation or a glycan equivalent thereof, and 45 wt % or less of one or more barley fiber preparation or a glycan equivalent thereof. In some embodiments, the amount of one or more citrus pectin or a glycan equivalent thereof is less than 1 wt %, or citrus pectin or a glycan equivalent thereof is absent from the composition. In further embodiments, the one or more citrus fiber preparation or a glycan equivalent thereof is in an amount that does not exceed 15 wt %, or in an amount that does not exceed 12 wt %. In still further embodiments, the one or more barley fiber preparation or a glycan equivalent thereof is in an amount that does not exceed 30 wt %, or in an amount that does not exceed 20 wt %. In still further embodiments, there is either (i) one or more pea fiber preparation or a glycan equivalent thereof, or (ii) one or more sugar beet fiber preparation or a glycan equivalent thereof. The glycan equivalent can be a functional glycan equivalent or a compositional glycan equivalent.

In some embodiments, a composition comprises (a) at least 28 wt % of one or more pea fiber preparation, at least 28 wt % of one or more sugar beet fiber preparation, or a glycan equivalent thereof, and (b) at least one additional fiber preparation chosen from: at least 28 wt % of one or more high molecular weight inulin preparation or a glycan equivalent thereof, 10 wt % of less of one or more citrus pectin preparation or a glycan equivalent thereof, 25 wt % or less of one or more citrus fiber preparation or a glycan equivalent thereof, and 45 wt % or less of one or more barley fiber preparation or a glycan equivalent thereof. In another embodiment, a composition consists essentially of (a) at least 28 wt % of one or more pea fiber preparation, at least 28 wt % of one or more sugar beet fiber preparation, or a glycan equivalent thereof, and (b) at least one additional fiber preparation chosen from: at least 28 wt % of one or more high molecular weight inulin preparation or a glycan equivalent thereof, 10 wt % of less of one or more citrus pectin preparation or a glycan equivalent thereof, 25 wt % or less of one or more citrus fiber preparation or a glycan equivalent thereof, and 45 wt % or less of one or more barley fiber preparation or a glycan equivalent thereof. In some embodiments, the amount of one or more citrus pectin preparation or a glycan equivalent thereof is less than 1 wt %, or citrus pectin or a glycan equivalent thereof is absent from the composition. In further embodiments, the one or more citrus fiber preparation or a glycan equivalent thereof is in an amount that does not exceed 15 wt %, or in an amount that does not exceed 12 wt %. In still further embodiments, the one or more barley fiber preparation or a glycan equivalent thereof is in an amount that does not exceed 30 wt %, or in an amount that does not exceed 20 wt %. In still further embodiments, there is either (i) one or more pea fiber preparation or a glycan equivalent thereof, or (ii) one or more sugar beet fiber preparation or a glycan equivalent thereof. The glycan equivalent can be a functional glycan equivalent or a compositional glycan equivalent.

In some embodiments, a composition comprises (a) at least 30 wt % of one or more pea fiber preparation, at least 30 wt % of one or more sugar beet fiber preparation, or a glycan equivalent thereof, and (b) at least one additional fiber preparation chosen from: at least 30 wt % of one or more high molecular weight inulin preparation or a glycan equivalent thereof, 10 wt % of less of one or more citrus pectin preparation or a glycan equivalent thereof, 25 wt % or less of one or more citrus fiber preparation or a glycan equivalent thereof, and 45 wt % or less of one or more barley fiber preparation or a glycan equivalent thereof. In another embodiment, a composition consists essentially of (a) at least 30 wt % of one or more pea fiber preparation, at least 30 wt % of one or more sugar beet fiber preparation, or a glycan equivalent thereof, and (b) at least one additional fiber preparation chosen from: at least 30 wt % of one or more high molecular weight inulin preparation or a glycan equivalent thereof, 10 wt % of less of one or more citrus pectin preparation or a glycan equivalent thereof, 25 wt % or less of one or more citrus fiber preparation or a glycan equivalent thereof, and 45 wt % or less of one or more barley fiber preparation or a glycan equivalent thereof. In some embodiments, the citrus pectin preparation or a glycan equivalent thereof is less than 1 wt %, or citrus pectin preparation or a glycan equivalent thereof is absent from the composition. In further embodiments, the one or more citrus fiber preparation or a glycan equivalent thereof is in an amount that does not exceed 15 wt %, or in an amount that does not exceed 12 wt %. In still further embodiments, the one or more barley fiber preparation or a glycan equivalent thereof is in an amount that does not exceed 30 wt %, or in an amount that does not exceed 20 wt %. In still further embodiments, there is either (i) one or more pea fiber preparation or a glycan equivalent thereof, or (ii) one or more sugar beet fiber preparation or a glycan equivalent thereof. The glycan equivalent can be a functional glycan equivalent or a compositional glycan equivalent.

In some embodiments, a composition comprises about 30 wt % to about 40 wt % of one or more pea fiber preparation or a glycan equivalent thereof, about 30 wt % to about 40 wt % of one or more high molecular weight inulin preparation or a glycan equivalent thereof, about 9 wt % to about 11 wt % of one or more citrus fiber preparation or a glycan equivalent thereof, and about 18 wt % to about 22 wt % of one or more barley fiber preparation or a glycan equivalent thereof. In another embodiment, a composition consists essentially of about 30 wt % to about 40 wt % of one or more pea fiber preparation or a glycan equivalent thereof, about 30 wt % to about 40 wt % of one or more high molecular weight inulin preparation or a glycan equivalent thereof, about 9 wt % to about 11 wt % of one or more citrus fiber preparation or a glycan equivalent thereof, and about 18 wt % to about 22 wt % of one or more barley fiber preparation or a glycan equivalent thereof. The glycan equivalent can be a functional glycan equivalent or a compositional glycan equivalent.

In some embodiments, a composition comprises about 30 wt % to about 35 wt % of one or more pea fiber preparation or a glycan equivalent thereof, about 35 wt % to about 40 wt % of one or more high molecular weight inulin preparation or a glycan equivalent thereof, about 9 wt % to about 11 wt % of one or more citrus fiber preparation or a glycan equivalent thereof, and about 18 wt % to about 22 wt % of one or more barley fiber preparation or a glycan equivalent thereof. In another embodiment, a composition consists essentially of about 30 wt % to about 35 wt % of one or more pea fiber preparation or a glycan equivalent thereof, about 35 wt % to about 40 wt % of one or more high molecular weight inulin preparation or a glycan equivalent thereof, about 9 wt % to about 11 wt % of one or more citrus fiber preparation or a glycan equivalent thereof, and about 18 wt % to about 22 wt % of one or more barley fiber preparation or a glycan equivalent thereof. The glycan equivalent can be a functional glycan equivalent or a compositional glycan equivalent.

In some embodiments, a composition comprises about 35 wt % of one or more pea fiber preparation or a glycan equivalent thereof, about 35 wt % of one or more high molecular weight inulin preparation or a glycan equivalent thereof, about 10 wt % of one or more citrus fiber preparation or a glycan equivalent thereof, and about 20 wt % of one or more barley fiber preparation or a glycan equivalent thereof. In another embodiment, a composition consists essentially of about 35 wt % of one or more pea fiber preparation or a glycan equivalent thereof, about 35 wt % of one or more high molecular weight inulin preparation or a glycan equivalent thereof, about 10 wt % of one or more citrus fiber preparation or a glycan equivalent thereof, and about 20 wt % of one or more barley fiber preparation or a glycan equivalent thereof. The glycan equivalent can be a functional glycan equivalent or a compositional glycan equivalent.

In some embodiments, a composition comprises about 33 wt % of one or more pea fiber preparation or a glycan equivalent thereof, about 36 wt % of one or more high molecular weight inulin preparation or a glycan equivalent thereof, about 11 wt % of one or more citrus fiber preparation or a glycan equivalent thereof, and about 20 wt % of one or more barley fiber preparation or a glycan equivalent thereof. In another embodiment, a composition consists essentially of about 33 wt % of one or more pea fiber preparation or a glycan equivalent thereof, about 36 wt % of one or more high molecular weight inulin preparation or a glycan equivalent thereof, about 11 wt % of one or more citrus fiber preparation or a glycan equivalent thereof, and about 20 wt % of one or more barley fiber preparation or a glycan equivalent thereof. The glycan equivalent can be a functional glycan equivalent or a compositional glycan equivalent.

In some embodiments, a composition comprises or consists essentially of about 60 wt % to about 70 wt % of one or more pea fiber preparation or a glycan equivalent thereof and about 30 wt % to about 40 wt % of one or more high molecular weight inulin preparation or a glycan equivalent thereof. The glycan equivalent can be a functional glycan equivalent or a compositional glycan equivalent.

In some embodiments, a composition comprises or consists essentially of about 65 wt % of one or more pea fiber preparation or a glycan equivalent thereof, about 35 wt % of one or more high molecular weight inulin preparation or a glycan equivalent thereof. The glycan equivalent can be a functional glycan equivalent or a compositional glycan equivalent.

Fiber preparations may be prepared from plant material by methods known in the art. Plant-derived fiber preparations that are economical for use in human foods typically are mixtures of diverse molecular composition comprising not only dietary fiber but also protein, fat, carbohydrate, etc. A skilled artisan will appreciate that fiber preparations prepared by different manufacturing processes may have different compositions, and a proximate analysis may be used to evaluate the suitability of a fiber preparation. A proximate analysis of a composition (e.g., a fiber preparation, a food item) refers to an analysis of the composition's moisture, protein, fat, ash, and carbohydrate content, which are expressed as the content (wt %) in the composition, respectively. Protein, fat, ash, and moisture content can be measured by methods established by Association of Official Analytical Chemists (AOAC) 2009.01, AOAC 920.123, AOAC 933.05, AOAC 935.42, and AOAC 926.08, respectively, and carbohydrate can be defined as (100−(Protein+Fat+Ash+Moisture). Analysis of the dietary fiber, which is measured separately, may provide further information by which to evaluate the suitability of a preparation. For instance, soluble and insoluble dietary fiber, and high molecular weight and low molecular weight dietary fiber, can be measured by AOAC method 2011.25. Further details are provided in the Examples. Suitable fiber preparations will be substantially similar to those disclosed herein. As demonstrated herein, a fiber preparation contains active and inactive fractions with different structural features and biophysical availability, from the perspective of the gut microbiota. Accordingly, preferred fiber preparations may also have substantially similar monosaccharide content and/or glycosidic linkages. Methods for measuring monosaccharide content and performing a glycosidic linkage analysis are known in the art, and described herein.

(a) Barley Fiber Preparations

Barley fiber preparations may be prepared according to methods known in the art, and evaluated as described herein. Commercial sources may also be used.

In some embodiments, a composition comprises one or more barley fiber preparation in an amount that does not exceed 45 wt % of the composition. The amount may also be expressed as individual values or a range. For instance, the barley fiber preparation(s) in these embodiments may be about 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, or 45 wt %. In some examples, the barley fiber preparation(s) may be about 1 wt % to about 45 wt %, about 10 wt % to about 45 wt %, or about 20 wt % to about 45 wt % of the composition. In some examples, the barley fiber preparation(s) may be about 1 wt % to about 25 wt % or about 10 wt % to about 25 wt % of the composition, or about 1 wt % to about 20 wt % or about 10 wt % to about 20 wt % of the composition.

In an exemplary embodiment of a suitable barley fiber preparation, the total dietary fiber is comprised of about 5 wt % to about 15 wt %, or about 10 wt % to about 15% of insoluble dietary fiber and/or about 40 wt % to about 50 wt %, or about 42 wt % to about 47 wt % of high molecular weight dietary fiber. In some embodiments, the total dietary fiber is about 35 wt % to about 55 wt %, about 40 wt % to about 55 wt %, or about 45 wt % to about 55 wt % of the preparation. In other embodiments, the total dietary fiber is about 35 wt % to about 50 wt % or about 30 wt % to about 45 wt % of the preparation. In still further embodiments, the barley fiber preparation comprises about 15 wt % to about 20 wt % protein, about 2 wt % to about 5 wt % fat, about 65 wt % to about 75 wt % carbohydrate, about 2 wt % to about 7 wt % moisture, and about 1 wt % to about 3 wt % ash.

In another exemplary embodiment of a suitable barley fiber preparation, the total dietary fiber is comprised of about 5 wt % to about 15 wt %, or about 10 wt % to about 15% of insoluble dietary fiber and about 40 wt % to about 50 wt %, or about 42 wt % to about 47 wt % of high molecular weight dietary fiber; the total dietary fiber is about 35 wt % to about 55 wt %, about 40 wt % to about 55 wt %, or about 45 wt % to about 55 wt % of the preparation; and the barley fiber preparation comprises about 15 wt % to about 20 wt % protein, about 2 wt % to about 5 wt % fat, about 65 wt % to about 75 wt % carbohydrate, about 2 wt % to about 7 wt % moisture, and about 1 wt % to about 3 wt % ash.

In another exemplary embodiment, a suitable barley fiber preparation is substantially similar to the preparation described in Table A.

In each of the above embodiments, a suitable barley fiber preparation may also have a monosaccharide content substantially similar to the preparation described in Table B, glycosidic linkages substantially similar to the preparation exemplified in Table E, or both.

In another exemplary embodiment, a suitable barley fiber preparation has a monosaccharide content substantially similar to the preparation exemplified in Table B and glycosyl linkages that are substantially similar to the preparation exemplified in Table E.

In another exemplary embodiment, a suitable barley fiber preparation is substantially similar to the preparation described in Table G.

(b) Citrus Fiber Preparations

Citrus fiber preparations may be prepared according to methods known in the art from citrus fruits including, but not limited to, clementine, citron, grapefruit, kumquat, lemon, lime, orange, tangelo, tangerine, and yuzu, and evaluated as described herein. Commercial sources may also be used.

In some embodiments, a composition comprises one or more citrus fiber preparation in an amount that does not exceed 25 wt % of the composition. The amount may also be expressed as individual values or a range. For instance, the citrus fiber preparation(s) in these embodiments may be about 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or 25 wt %. In some examples, the citrus fiber preparation(s) may be about 1 wt % to about 25 wt %, about 1 wt % to about 20 wt %, or about 1 wt % to about 15 wt % of the composition. In some examples, the citrus fiber preparation(s) may be about 5 wt % to about 25 wt %, about 5 wt % to about 20 wt %, or about 5 wt % to about 15 wt % of the composition. In some examples, the citrus fiber preparation(s) may be about 10 wt % to about 25 wt %, about 10 wt % to about 20 wt %, or about 10 wt % to about 15 wt % of the composition.

In an exemplary embodiment of a suitable citrus fiber preparation, the total dietary fiber is comprised of about 30 wt % to about 40 wt %, or about 30 wt % to about 35% of insoluble dietary fiber and/or about 65 wt % to about 75 wt %, or about 65 wt % to about 70 wt % of high molecular weight dietary fiber. In some embodiments, the total dietary fiber is about 60 wt % to about 80 wt %, about 60 wt % to about 75 wt %, or about 60 wt % to about 70 wt % of the preparation. In other embodiments, the total dietary fiber is about 65 wt % to about 80 wt %, about 65 wt % to about 75 wt %, or about 65 wt % to about 70 wt % of the preparation. In still further embodiments, the citrus fiber preparation comprises about 5 wt % to about 10 wt % protein, about 1 wt % to about 3 wt % fat, about 75 wt % to about 85 wt % carbohydrate, about 5 wt % to about 10 wt % moisture, and about 1 wt % to about 4 wt % ash.

In another exemplary embodiment of a suitable citrus fiber preparation, the total dietary fiber is comprised of about 30 wt % to about 40 wt %, or about 30 wt % to about 35% of insoluble dietary fiber and/or about 65 wt % to about 75 wt %, or about 65 wt % to about 70 wt % of high molecular weight dietary fiber; the total dietary fiber is about 65 wt % to about 80 wt %, about 65 wt % to about 75 wt %, or about 65 wt % to about 70 wt % of the preparation; and the citrus fiber preparation comprises about 5 wt % to about 10 wt % protein, about 1 wt % to about 3 wt % fat, about 75 wt % to about 85 wt % carbohydrate, about 5 wt % to about 10 wt % moisture, and about 1 wt % to about 4 wt % ash.

In another exemplary embodiment, a suitable citrus fiber preparation is substantially similar to the preparation described in Table A.

In each of the above embodiments, a suitable citrus fiber preparation may also have monosaccharide content substantially similar to a preparation described in Table B, glycosidic linkages substantially similar to a preparation exemplified in Table F1 or F2, or both.

In another exemplary embodiment, a suitable citrus fiber preparation has a monosaccharide content is substantially similar to a preparation exemplified in Table B and glycosyl linkages that are substantially similar to a preparation exemplified in Table F1 or F2.

In another exemplary embodiment, a suitable citrus fiber preparation is substantially similar to the preparation described in Table G

(c) Citrus Pectin Preparations

Citrus pectin preparations may be prepared according to methods known in the art from citrus fruits including, but not limited to, clementine, citron, grapefruit, kumquat, lemon, lime, orange, tangelo, tangerine, and yuzu, and evaluated as described herein. Commercial sources may also be used.

In some embodiments, a composition comprises one or more citrus pectin preparation in an amount that does not exceed 10 wt % of the composition. The amount may also be expressed as individual values or a range. For instance, the amount of citrus pectin in these embodiments may be about 1 wt %, 2 wt %, 3 wt %, 4 wt %, 5 wt %, 6 wt %, 7 wt %, 8 wt %, 9 wt %, or 10 wt %. In some examples, the citrus pectin preparation(s) may be about 1 wt % to about 10 wt %, about 1 wt % to about 8 wt %, or about 1 wt % to about 6 wt % of the composition. In some examples, the citrus pectin preparation(s) may be about 1 wt % to about 4 wt %, or about 1 wt % to about 2 wt % of the composition.

In an exemplary embodiment of a suitable citrus pectin preparation, the total dietary fiber is comprised of about 1 wt % to about 10 wt %, or about 1 wt % to about 5% of insoluble dietary fiber and/or about 85 wt % to about 95 wt %, or about 90 wt % to about 95 wt % of high molecular weight dietary fiber. In some embodiments, the total dietary fiber is about 75 wt % to about 95 wt %, about 80 wt % to about 95 wt %, or about 85 wt % to about 95 wt % of the preparation. In other embodiments, the total dietary fiber is about 85 wt % to about 90 wt % or about 90 wt % to about 95 wt % of the preparation. In still further embodiments, the citrus pectin preparation comprises about 2 wt % or less of protein, about 1 wt % to about 2 wt % fat, about 85 wt % to about 95 wt % carbohydrate, about 1 wt % to about 6 wt % moisture, and about 3 wt % to about 6 wt % ash.

In another exemplary embodiment of a suitable citrus pectin preparation, the total dietary fiber is comprised of about 1 wt % to about 10 wt %, or about 1 wt % to about 5% of insoluble dietary fiber and about 85 wt % to about 95 wt %, or about 90 wt % to about 95 wt % of high molecular weight dietary fiber; the total dietary fiber is about 85 wt % to about 95 wt %, about 85 wt % to about 90 wt %, or about 90 wt % to about 95 wt % of the preparation; and the citrus pectin preparation comprises about 2 wt % or less of protein, about 1 wt % to about 2 wt % fat, about 85 wt % to about 95 wt % carbohydrate, about 1 wt % to about 6 wt % moisture, and about 3 wt % to about 6 wt % ash.

In another exemplary embodiment, a suitable citrus pectin preparation is substantially similar to the preparation described in Table A.

In each of the above embodiments, a suitable citrus pectin preparation may also have a monosaccharide content substantially similar to the preparation exemplified in Table B, glycosyl linkages substantially similar to the preparation exemplified in Table D, or both.

In another exemplary embodiment, a suitable citrus pectin preparation has a monosaccharide content substantially similar to the preparation exemplified in Table B and glycosyl linkages that are substantially similar to the preparation exemplified in Table D.

(d) High Molecular Weight Inulin Preparations

High molecular weight inulin preparations may be prepared according to methods known in the art, and evaluated as described herein. Commercial sources may also be used. Inulin is defined by AOAC method 999.03. High molecular weight inulin is comprised of fructose units linked together by ß-(2,1)-linkages, which are typically terminated by a glucose unit.

In some embodiments, a composition comprises one or more high molecular weight inulin preparation in an amount that is at least 28 wt % of the composition. The amount may also be expressed as individual values or a range. For instance, the high molecular weight inulin preparation(s) in these embodiments may be about 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, or more. In some examples, the high molecular weight inulin preparation(s) may be about 30 wt % to about 50 wt %, about 30 wt % to about 45 wt %, or about 30 wt % to about 40 wt % of the composition. In some examples, the high molecular weight inulin preparation(s) may be about 35 wt % to about 50 wt %, about 35 wt % to about 45 wt %, or about 35 wt % to about 40 wt % of the composition. Inulin is defined by AOAC method 999.03.

In an exemplary embodiment of a suitable high molecular weight inulin preparation, the total dietary fiber is comprised of about 0.5 wt % or less of insoluble dietary fiber and/or about 55 wt % to about 65 wt %, or about 57 wt % to about 62 wt % of high molecular weight dietary fiber. In some embodiments, the total dietary fiber is about 75 wt % to about 95 wt %, about 80 wt % to about 95 wt %, or about 85 wt % to about 95 wt % of the preparation. In other embodiments, the total dietary fiber is about 85 wt % to about 99 wt %, 90 wt % to about 99 wt %, or about 95 wt % to about 99 wt % of the preparation. In still further embodiments, the high molecular weight inulin preparation comprises no more than 1 wt % of protein, about 2 wt % to about 5 wt % fat, about 85 wt % to about 95 wt % carbohydrate, about 2 wt % to about 7 wt % moisture, and no more than 2 wt % ash.

In an exemplary embodiment of a suitable high molecular weight inulin preparation, the total dietary fiber is comprised of about 0.5 wt % insoluble dietary fiber and about 55 wt % to about 65 wt %, or about 57 wt % to about 62 wt % of high molecular weight dietary fiber; the total dietary fiber is about 85 wt % to about 99 wt %, 90 wt % to about 99 wt %, or about 95 wt % to about 99 wt % of the preparation; and the high molecular weight inulin preparation comprises no more than 1 wt % of protein, about 2 wt % to about 5 wt % fat, about 85 wt % to about 95 wt % carbohydrate, about 2 wt % to about 7 wt % moisture, and no more than 2 wt % ash.

In another exemplary embodiment, a suitable high molecular weight inulin preparation is substantially similar to the preparation described in Table A.

In another exemplary embodiment, a suitable high molecular weight inulin preparation is substantially similar to the preparation described in Table G.

In each of the above embodiments, about 99% of the inulin in a suitable high molecular weight inulin preparation may have a degree of polymerization (DP) that is greater than or equal to 5. In some example, the DP for the inulin in a suitable preparation may range from 5 to 60. Alternatively or in addition, the average DP may be less than or equal to 23.

(e) Pea Fiber Preparations

Pea fiber preparations may be prepared according to methods known in the art, and evaluated as described herein. Commercial sources may also be used.

In some embodiments, a composition comprises one or more pea fiber preparation in an amount that is at least 15 wt % of the composition. The amount may also be expressed as individual values or a range. For instance, the pea fiber preparation(s) in these embodiments may be about 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, 51 wt %, 52 wt %, 53 wt %, 54 wt %, 55 wt %, 56 wt %, 57 wt %, 58 wt %, 59 wt %, 60 wt %, 61 wt %, 62 wt %, 63 wt %, 64 wt %, 65 wt %, or more. In some examples, the pea fiber preparation(s) may be about 15 wt % to about 75 wt %, about 25 wt % to about 75 wt %, or about 35 wt % to about 75 wt % of the composition. In some examples, the pea fiber preparation(s) may be about 15 wt % to about 65 wt %, about 25 wt % to about 65 wt %, or about 35 wt % to about 65 wt % of the composition. In some examples, the pea fiber preparation(s) may be about 30 wt % to about 85 wt %, about 40 wt % to about 85 wt %, or about 50 wt % to about 85 wt % of the composition.

In an exemplary embodiment of a suitable pea fiber preparation, the total dietary fiber is comprised of about 55 wt % to about 65 wt %, or about 60 wt % to about 65% of insoluble dietary fiber and/or about 60 wt % to about 70 wt %, or about 65 wt % to about 70 wt % of high molecular weight dietary fiber. In some embodiments, the total dietary fiber is about 60 wt % to about 80 wt %, about 60 wt % to about 75 wt %, or about 60 wt % to about 70 wt % of the preparation. In other embodiments, the total dietary fiber is about 65 wt % to about 80 wt %, about 65 wt % to about 75 wt %, or about 65 wt % to about 70 wt % of the preparation. In still further embodiments, the pea fiber preparation comprises about 7 wt % to about 12 wt % protein, no more than 2 wt % fat, about 75 wt % to about 85 wt % carbohydrate, about 5 wt % to about 10 wt % moisture, and about 1 wt % to about 4 wt % ash.

In an exemplary embodiment of a suitable pea fiber preparation, the total dietary fiber is comprised of about 55 wt % to about 65 wt %, or about 60 wt % to about 65% of insoluble dietary fiber and about 60 wt % to about 70 wt %, or about 65 wt % to about 70 wt % of high molecular weight dietary fiber; the total dietary fiber is about 65 wt % to about 80 wt %, about 65 wt % to about 75 wt %, or about 65 wt % to about 70 wt % of the preparation; and the pea fiber preparation comprises about 7 wt % to about 12 wt % protein, no more than 2 wt % fat, about 75 wt % to about 85 wt % carbohydrate, about 5 wt % to about 10 wt % moisture, and about 1 wt % to about 4 wt % ash.

In another exemplary embodiment, a suitable pea fiber preparation is substantially similar to the preparation described in Table A.

In each of the above embodiments, a suitable pea fiber preparation may also have a monosaccharide content substantially similar to a preparation exemplified in Table B; glycosyl linkages substantially similar to the preparation exemplified in Table C1, Table C2, Table 13, Table 14, Table 16, or Table 17; or both.

In another exemplary embodiment, a suitable pea fiber preparation has a monosaccharide content substantially similar to a preparation exemplified in Table B and glycosyl linkages substantially similar to the preparation exemplified in Table C1, Table C2, Table 13, Table 14, Table 16, or Table 17.

In another exemplary embodiment a suitable pea fiber preparation has a monosaccharide content that has about 10 wt % to about 90 wt % arabinose, and arabinose linkages that are substantially similar to the preparation exemplified in Table C1, Table C2, Table 13, Table 14, Table 16, or Table 17. In some examples, arabinose may be about 10 wt % to 20 wt %, or about 15 wt % to about 20 wt %. In some examples, arabinose may be about 20 wt % to 30 wt %, about 20 wt % to about 25 wt %, or about 25 wt % to about 30 wt %. In some examples, arabinose may be about 50 wt % to 90 wt %, about 60 wt % to about 90 wt %, or about 70 wt % to about 90 wt %. In some examples, arabinose may be about 50 wt % to 80 wt %, about 60 wt % to about 80 wt %, or about 70 wt % to about 80 wt %.

In another exemplary embodiment, a suitable pea fiber preparation has a monosaccharide content that has a substantially similar arabinose content as the preparation exemplified in Table B and arabinose glycosyl linkages that are substantially similar to the preparation exemplified in Table C1, Table C2, Table 13, Table 14, Table 16, or Table 17.

In another exemplary embodiment, a suitable pea fiber preparation is substantially similar to the Fiber 8 fraction or the enzymatically destarched Fiber 8 fraction described in Example 10.

In another exemplary embodiment, a suitable pea fiber preparation is substantially similar to the preparation described in Table G.

In all the aforementioned embodiments, a suitable pea fiber preparation may also comprise arabinan of formula (I):

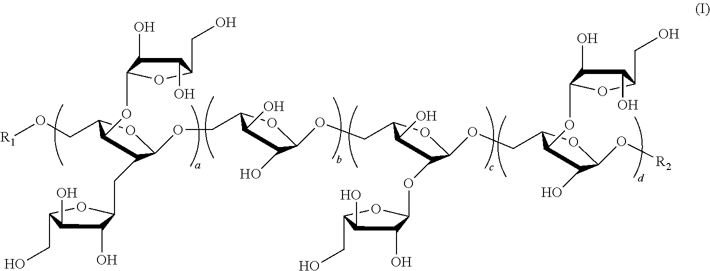

wherein a is about 0.1 to about 0.3, b is about 0.4 to about 0.6, c is about 0.1 to about 0.4, d is about 0.04 to about 0.06 (calculated from the fractional abundance of arabinose linkages where the arabinose contained a 5-linkage, as determined by partially methylated alditol acetate GC-MS analysis); and wherein R₁ and R₂ are each independently selected from H, a glycosyl, a sugar moiety (modified or not), an oligosaccharide (branched or not), or a polysaccharide (branched or not), and a polysaccharide containing galacturonic acid, galactose, and rhamnose.

Alternatively, in all the aforementioned embodiments, a suitable pea fiber preparation may also comprise arabinan of formula (I):

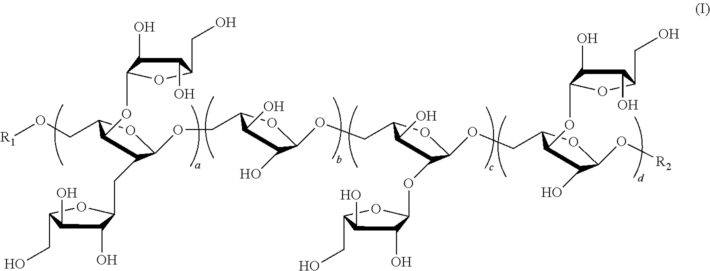

wherein a is about 0.2 to about 0.3, b is about 0.5 to about 0.6, c is about 0.2 to about 0.4, d is about 0.04 to about 0.06 (calculated from the fractional abundance of arabinose linkages where the arabinose contained a 5-linkage, as determined by partially methylated alditol acetate GC-MS analysis); and wherein R₁ and R₂ are each independently selected from H, a glycosyl, a sugar moiety (modified or not), an oligosaccharide (branched or not), or a polysaccharide (branched or not), and a polysaccharide containing galacturonic acid, galactose, and rhamnose.

Alternatively, in all the aforementioned embodiments, a suitable pea fiber preparation may also comprise arabinan of formula (I):

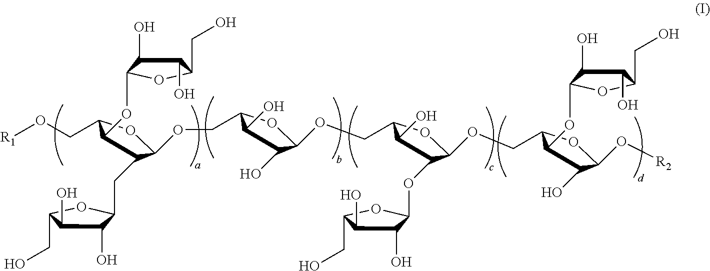

wherein a is about 0.1 to about 0.2, b is about 0.4 to about 0.5, c is about 0.2 to about 0.4, d is about 0.04 to about 0.06 (calculated from the fractional abundance of arabinose linkages where the arabinose contained a 5-linkage, as determined by partially methylated alditol acetate GC-MS analysis); and wherein R₁ and R₂ are each independently selected from H, a glycosyl, a sugar moiety (modified or not), an oligosaccharide (branched or not), or a polysaccharide (branched or not), and a polysaccharide containing galacturonic acid, galactose, and rhamnose.

Alternatively, in all the aforementioned embodiments, a suitable pea fiber preparation may also comprise arabinan of formula (I):

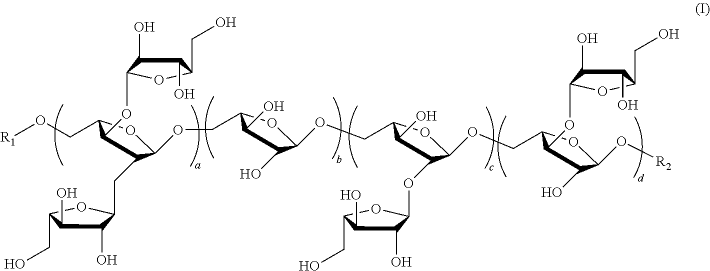

wherein a is about 0.2 to about 0.3, b is about 0.4 to about 0.5, c is about 0.3 to about 0.4, d is about 0.04 to about 0.06 (calculated from the fractional abundance of arabinose linkages where the arabinose contained a 5-linkage, as determined by partially methylated alditol acetate GC-MS analysis); wherein R₁ and R₂ are each independently selected from H, a glycosyl, a sugar moiety (modified or not), an oligosaccharide (branched or not), or a polysaccharide (branched or not), and a polysaccharide containing galacturonic acid, galactose, and rhamnose.

Alternatively, in all the aforementioned embodiments, a suitable pea fiber preparation may also comprise arabinan of formula (I):

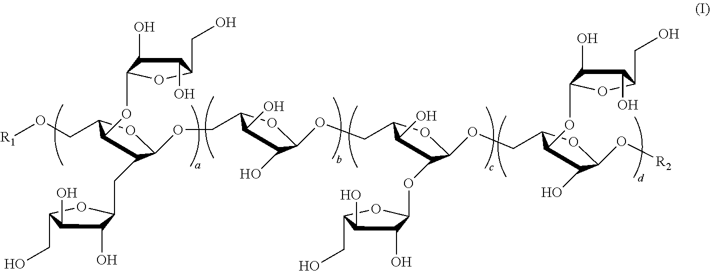

wherein a is about 0.20, b is about 0.47, c is about 0.28, d is about 0.05 (calculated from the fractional abundance of arabinose linkages where the arabinose contained a 5-linkage, as determined by partially methylated alditol acetate GC-MS analysis); wherein R₁ and R₂ are each independently selected from H, a glycosyl, a sugar moiety (modified or not), an oligosaccharide (branched or not), or a polysaccharide (branched or not), and a polysaccharide containing galacturonic acid, galactose, and rhamnose.

The molecular weight of the arabinan may be about 2 kDa to about 500,000 kDa, or more. In one example, the molecular weight of the arabinan may be about 1000 kDa to about 500,000 kDa. In one example, the molecular weight of the arabinan may be about 1000 kDa to about 200,000 kDa. In one example, the molecular weight of the arabinan may be about 1000 kDa to about 100,000 kDa. In one example, the molecular weight of the arabinan may be about 1000 kDa to about 10,000 kDa. In one example, the molecular weight of the arabinan may be about 10,000 kDa to about 500,000 kDa. In one example, the molecular weight of the arabinan may be about 10,000 kDa to about 200,000 kDa. In one example, the molecular weight of the arabinan may be about 100,000 kDa to about 500,000 kDa.

The total amount of all arabinans of formula (I) in a suitable pea fiber preparation may vary. In some embodiments, the total amount may be at least 10 wt %. For example, the total amount may be about 10 wt %, about 15 wt %, about 20 wt %, about 25 wt %, about 30 wt %, about 35 wt %, about 40 wt %, about 45 wt %, about 50 wt %, about 55 wt %, about 60 wt %, about 65 wt %, about 70 wt %, about 75 wt %, about 80 wt %, about 85 wt %, about 90 wt %, about 95 wt %. In some embodiments, the total amount may be at least 20 wt %, at least 30 wt %, at least 40 wt %, at least 50 wt %, at least, 60 wt %, at least, 70 wt %, at least, 80 wt %, at least 90 wt %. In some embodiments, the total amount may be about 10 wt % to about 50 wt %, about 20 wt % to about 50 wt %, about 30 wt % to about 50 wt %, about 40 wt % to about 50 wt %. In some embodiments, the total amount may be about 30 wt % to about 70 wt %, about 40 wt % to about 70 wt %, about 50 wt % to about 70 wt %, about 60 wt % to about 70 wt %. In some embodiments, the total amount may be about 50 wt % to about 90 wt %, about 60 wt % to about 90 wt %, about 70 wt % to about 90 wt %, about 80 wt % to about 90 wt %.

(f) Sugar Beet Fiber Preparations

Sugar beet fiber preparations may be prepared according to methods known in the art, and evaluated as described herein. Commercial sources may also be used.

In some embodiments, a composition comprises one or more sugar beet fiber preparation in an amount that is at least 15 wt % of the composition. The amount may also be expressed as individual values or a range. For instance, the pea fiber preparation(s) in these embodiments may be about 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, 51 wt %, 52 wt %, 53 wt %, 54 wt %, 55 wt %, 56 wt %, 57 wt %, 58 wt %, 59 wt %, 60 wt %, 61 wt %, 62 wt %, 63 wt %, 64 wt %, 65 wt %, or more. In some examples, the sugar beet fiber preparation(s) may be about 15 wt % to about 65 wt %, about 25 wt % to about 65 wt %, or about 35 wt % to about 65 wt % of the composition. In some examples, the sugar beet fiber preparation(s) may be about 15 wt % to about 55 wt %, about 25 wt % to about 55 wt %, or about 35 wt % to about 55 wt % of the composition. In some examples, the sugar beet fiber preparation(s) may be about 15 wt % to about 45 wt %, about 25 wt % to about 45 wt %, or about 35 wt % to about 45 wt % of the composition.

In an exemplary embodiment of a suitable sugar beet fiber preparation, the total dietary fiber is comprised of about 55 wt % to about 65 wt %, or about 60 wt % to about 65% of insoluble dietary fiber and/or about 75 wt % to about 85 wt %, or about 80 wt % to about 85 wt % of high molecular weight dietary fiber. In some embodiments, the total dietary fiber is about 70 wt % to about 90 wt %, about 70 wt % to about 85 wt %, or about 70 wt % to about 80 wt % of the preparation. In other embodiments, the total dietary fiber is about 75 wt % to about 90 wt %, about 80 wt % to about 90 wt %, or about 80 wt % to about 85 wt % of the preparation. In still further embodiments, the sugar beet fiber preparation comprises about 7 wt % to about 12 wt % protein, about 1 wt % to about 3 wt % fat, about 75 wt % to about 85 wt % carbohydrate, about 5 wt % to about 10 wt % moisture, and about 3 wt % to about 6 wt % ash.

In an exemplary embodiment of a suitable sugar beet fiber preparation, the total dietary fiber is comprised of about 55 wt % to about 65 wt %, or about 60 wt % to about 65% of insoluble dietary fiber and about 75 wt % to about 85 wt %, or about 80 wt % to about 85 wt % of high molecular weight dietary fiber, the total dietary fiber is about 75 wt % to about 90 wt %, about 80 wt % to about 90 wt %, or about 80 wt % to about 85 wt % of the preparation; and the sugar beet fiber preparation comprises about 7 wt % to about 12 wt % protein, about 1 wt % to about 3 wt % fat, about 75 wt % to about 85 wt % carbohydrate, about 5 wt % to about 10 wt % moisture, and about 3 wt % to about 6 wt % ash.

In another exemplary embodiment, a suitable sugar beet preparation is substantially similar to the preparation described in Table A.

(g) Glycan Equivalents

In each of the above embodiments, a compositional glycan equivalent thereof and/or a functional glycan equivalent thereof may be used as an alternative for a barley fiber preparation, a citrus fiber preparation, a citrus pectin preparation, a high molecular weight inulin preparation, a pea fiber preparation, and/or a sugar beet fiber preparation.

In some embodiments, a suitable functional glycan equivalent of a barley fiber preparation, a citrus fiber preparation, a citrus pectin preparation, a high molecular weight inulin preparation, a pea fiber preparation, or a sugar beet fiber preparation has a substantially similar function as a respective preparation identified in Table 2A. Substantially similar function may be measured by any one or more method detailed in the Examples herein, in particular the ability to affect relative or total abundances of microbial community members, in particular primary and secondary fiber degrading microbes, more particularly Bacteroides species; and/or expression of one or more microbial genes or gene product, in particular one or more gene or gene product encoded by polysaccharide utilization loci (PULs) and/or one or more CAZyme. In an exemplary embodiment, a suitable functional glycan equivalent is a fiber preparation that is enriched for one or more bioactive glycan, as compared to a barley fiber preparation, a citrus fiber preparation, a citrus pectin preparation, a high molecular weight inulin preparation, a pea fiber preparation, or a sugar beet fiber preparation used in the Examples.

For instance, a suitable functional glycan equivalent of a fiber preparation may have a similar effect on the relative abundance of Bacteroides species in a subject's gut microbiota. In another example, a suitable functional glycan equivalent of a fiber preparation may have a similar effect on the total abundance of Bacteroides species in a subject's gut microbiota. In another example, a suitable functional glycan equivalent of a fiber preparation may have a similar effect on the relative abundance of a subset of Bacteroides species. In another example, a suitable functional glycan equivalent of a fiber preparation may have a similar effect on the total abundance of a subset of Bacteroides species. In one example, the subset of Bacteroides species may include one or more species chosen from B. caccae, B. cellulosilyticus, B. finegoldii, B. massiliensis, B. ovatus, B. thetaiotaomicron, and B. vulgatus. In another example, a suitable functional glycan equivalent may have a similar effect on the relative abundance of one or more species chosen from Bacteroides ovatus, Bacteroides cellulosilyticus, Bacteroides thetaiotaomicron, Bacteroides vulgatus, Bacteroides caccae, Bacteroides finegoldfi, Bacteroides massiliensis, Collinsella aerofaciens, Escherichia coli, Odoribacter splanchnicus, Parabacteroides distasonis, a Ruminococcaceae sp., and Subdoligranulum variabile.

Alternatively or in addition, a suitable functional glycan equivalent may have a similar effect on the abundance or activity of one or more protein encoded by one or more polysaccharide utilization locus (PUL) and/or one or more CAZyme. In some examples, the PULs are chosen from PUL5, PULE, PUL7, PUL27, PUL31, PUL34, PUL35, PUL38, PUL42, PUL43, PUL73, PUL75, PUL83, and PUL97.

Although the Examples utilize a gnotobiotic mouse model where the mouse is colonized with a defined consortium of cultured, sequenced gut bacteria, the methods detailed in the Examples may also be used to measure effects in a gnotobiotic mouse model where the mouse is colonized with intact uncultured gut microbiota obtained from human(s), as well as to measure effects directly in humans.

(h) Additional Food Ingredients

In each of the above embodiments, the remaining weight percent (if any) of the composition is comprised of one or more additional food ingredients. Non-limiting examples include anti-caking agents, preservatives, pH control agents, color additives, flavors, flavor enhancers, and the like.

(i) Food Compositions

The present disclosure also provides food compositions comprising a composition of this section. The food composition may further comprise one or more additional food ingredients including, but not limited to, flours, meals, sweeteners, preservatives, color additives, flavors, spices, flavor enhancers, fats, oils, fat replacers (including components of formulations used to replace fats), nutrients, vitamins, minerals, emulsifiers, stabilizers, thickeners, binders, texturizers, pH control agents, leavening agents, anti-caking agents, humectants, firming agents, probiotics, and enzyme preparations, as well as inclusions, fruits, vegetables and grains.

Flours or meals may be made from a variety of sources, including but not limited to grains, legumes, roots, nuts or seeds.

Non-limiting examples of sweeteners include sucrose (sugar), glucose, fructose, sugar polyols (e.g., sorbitol, mannitol, etc.), syrups (e.g., corn syrup, high fructose corn syrup, etc.,) saccharin, aspartame, sucralose, acesulfame potassium (acesulfame-K), and neotame.

Preservatives include but are not limited to ascorbic acid, citric acid, sodium benzoate, calcium propionate, sodium erythorbate, sodium nitrite, calcium sorbate, potassium sorbate, BHA, BHT, EDTA, and tocopherols (Vitamin E).

Inclusions are substitutional or interstitial ingredients in the composition matrix. Non-limiting examples include candies, chips (chocolate, butterscotch, etc.), nuts, seeds, herbs, and the like.

Flavors may be natural, synthetic or artificial. Non-limiting examples of flavor enhancers include Monosodium glutamate (MSG), hydrolyzed soy protein, autolyzed yeast extract, disodium guanylate and inosinate.

Non-limiting examples of fat replacers include olestra, cellulose gel, carrageenan, polydextrose, modified food starch, microparticulated egg white protein, guar gum, xanthan gum, and whey protein concentrate. Emulsifiers may include lecithin, mono- and diglycerides, egg yolks, polysorbates, sorbitan monostearate, and glycerol monostearate.

Non-limiting examples of stabilizers, thickeners, binders, and texturizers include gelatin, pectin, guar gum, carrageenan, xanthan gum, and whey. Leavening agents include but are not limited to baking soda, monocalcium phosphate, calcium carbonate, ammonium bicarbonate, mono calcium phosphate monohydrate, sodium acid pyrophosphate, sodium aluminum phosphate, organic acids, and yeast. Humectants may be glycerin, sorbitol, and the like. Non-limiting examples of firming agents include calcium chloride and calcium lactate.

The amount of the composition in the food may vary. In some embodiments, a composition of this section may be about 5 wt % to about 60 wt % of the ingredients used to make the food (excluding any added water). In some embodiments, a composition of this section may be about 40 wt % to about 60 wt % of the ingredients used to make the food (excluding any added water). In some embodiments, a composition of this section may be about 40 wt % to about 50% wt %, about 45 wt % to about 50 wt %, or about 50 wt % to about 60 wt % of the ingredients used to make the food (excluding any added water). In some embodiments, a composition of this section may be about 45 wt % to about 50 wt % of the ingredients used to make the food (excluding any added water).

In certain embodiments, a composition of this section provides about 90% or more of the total dietary fiber in the food composition. For instance, the composition may provide about 90%, about 91%, about 92%, about 93%, about 94%, about 95%, about 96%, about 97%, about 98%, about 99%, or about 100% of the dietary fiber in the food composition. In one example, the composition provides about 95% or more of the total dietary fiber in the food composition. In another example, the composition provides about 98% or more of the total dietary fiber in the food composition.

In further embodiments, the food composition provides at least 6 g of dietary fiber per serving. In some examples, the food composition may provide at least 7 grams, at least 8 grams, at least 9 grams, or at least 10 grams of dietary fiber per serving. In other examples, the food composition may provide about 6 g to about 20 g, about 6 g to about 15 g, or about 6 g to about 10 g of dietary fiber per serving. A serving size may be at least 6 grams, for instance about 10 grams, about 15 grams, about 20 grams, about 25 grams, about 30 grams, about 35 grams, about 40 grams, about 45 grams, about 50 grams, etc. In certain embodiments, a serving is about 30 grams.

In some embodiments, a food composition is in a baked form. In some embodiments, a food composition is in a pressed or extruded form. In some embodiments, a food composition is in a powder form which may be reconstituted or sprinkled on a different food. In some embodiments, a food composition is a bar; a drink; a gel, a gummy, a candy, or the like; a cookie, a cracker, a cake, or the like; a dairy product (e.g., yogurt, ice cream or the like).

(j) Alternative Forms of Administration

The present disclosure also provides other oral dosage forms comprising a composition of this section. Suitable dosage forms include a tablet, including a suspension tablet, a chewable tablet, an effervescent tablet or caplet; a pill; a powder such as a sterile packaged powder, a dispensable powder, and an effervescent powder; a capsule including both soft or hard gelatin capsules such as HPMC capsules; a lozenge; pellets; granules; liquids; suspensions; emulsions; or semisolids and gels. Capsule and tablet formulations may include, but are not limited to binders, lubricants, and diluents. Capsules and tablets may be coated according to methods well known in the art. Aqueous suspension formulations may include but are not limited to dispersants, flavor-modifying agents, taste-masking agents, and coloring agents.

TABLE A
Compositional Analysis of Exemplary Fiber Preparations

| | % | % | % | | | | | | | |
|---|---|---|---|---|---|---|---|---|---|---|
| Fiber | Total | Insoluble | Soluble | HMW | LMW | % | % | % | % | % |
| Preparation | DF | DF | DF | DF | DF | Protein | Fat | Carb | Moisture | Ash |
| Barley bran | 46 | 11.1 | 20.8 | 45.2 | 0.9 | 18.72 | 4.13 | 69.28 | 5.69 | 1.96 |
| Citrus fiber | 68.5 | 33.2 | 29.5 | 68.5 | 0.6 | 7.47 | 2.16 | 80.92 | 5.69 | 1.96 |
| Citrus pectin | 91 | 4.7 | 85.3 | 91 | 0.6 | 0.61 | 1.23 | 90.07 | 3.51 | 4.58 |
| HMW inulin | 98.5 | <0.5 | 98.5 | 86 | 12.5 | 0.28 | 3.71 | 91.44 | 4.28 | 0.29 |
| Pea fiber | 67.2 | 61.4 | 4.9 | 66.3 | 0.8 | 9.49 | 0.93 | 79.75 | 7.37 | 2.46 |
| Sugar beet | 83.2 | 61.6 | 20.4 | 82 | 1.1 | 8.5 | 2.45 | 77.97 | 6.66 | 4.42 |

DF = dietary fiber,
HMW = high molecular weight,
LMW = low molecular weight
Protein, fat, ash, and moisture content are measured by methods established by Association of Official Analytical Chemists (AOAC) 2009.01, AOAC 920.123, AOAC 933.05, AOAC 935.42, and AOAC 926.08, respectively.
Carbohydrate is calculated as (100 − (Protein + Fat + Ash + Moisture). Total dietary fiber is measured by AOAC method 2009.01. Soluble and insoluble dietary fiber, and high molecular weight and low molecular weight dietary fiber, are measured by AOAC method 2011.25.

TABLE B
Monosaccharide Analysis of Fiber Preparations

| | | | Citrus fiber | | Citrus fiber | | Citrus | | Pea fiber | | Pea fiber | |
|---|---|---|---|---|---|---|---|---|---|---|---|---|
| | Barley fiber | | (raw) | | (extruded) | | pectin | | (raw) | | (extruded) | |
| | 2 h | 6 h | 2 h | 6 h | 2 h | 6 h | 2 h | 6 h | 2 h | 6h | 2 h | 6 h |
| Rhamnose | 0 | 0.4 | 1 | 1.8 | 1.3 | 1.6 | 0.8 | 1.5 | 0 | 1.7 | 0.9 | 1.1 |
| Fucose | 0 | 0 | 0 | 0.3 | 0 | 0.2 | 0 | 0 | 0 | 0.1 | 0 | 0 |
| Arabinose | 4.5 | 5.3 | 9.9 | 10.7 | 8.3 | 7.3 | 4.1 | 3.8 | 17.3 | 20.4 | 13.8 | 13.5 |
| Xylose | 6.1 | 7.4 | 2.3 | 2.7 | 2.4 | 2.2 | 0 | 0 | 4.8 | 5.6 | 3.3 | 3.2 |
| Mannose | 0.9 | 0.9 | 2.7 | 3.2 | 2.7 | 2.9 | 1.4 | 3.9 | 0.5 | 0.5 | 0.5 | 0.6 |
| Galactose | 0.5 | 0.5 | 4.5 | 5.0 | 3.8 | 3.6 | 4.1 | 4.1 | 2.6 | 3.0 | 2.3 | 2.2 |
| Glucose | 48.9 | 60.0 | 17.5 | 20.4 | 32.8 | 30.3 | 0.4 | 0.3 | 38.9 | 46.9 | 47.3 | 44.3 |
| Uronic acids | 0.8 | 0 | 45.9 | 33.1 | 32.2 | 26.3 | 78.8 | 80.6 | 13.4 | 12.7 | 9.5 | 8.8 |
| Total | 61.7 | 74.5 | 83.8 | 77.1 | 83.4 | 74.4 | 89.6 | 94.0 | 77.5 | 91.1 | 77.6 | 73.7 |
| Carbohydrates | | | | | | | | | | | | |

| | | | | | | |
|---|---|---|---|---|---|---|
| Water | 5.7 | 7.2 | 4.5 | 8.0 | 7.4 | 6.6 |
| Degree of | 0 | 29 | 45 | 72 | 16 | 20 |
| methylation (%) | | | | | | |
| Starch | 22.0 | ND | ND | ND | 16.6 | 28.5 |
| Beta-glucans | 17.0 | ND | ND | ND | ND | ND |
| Cellulose | 6.7 | 16.1 | 8.6 | 0 | 28.1 | 13.3 |

ND = none detected
Monosaccharide Method: Sugar composition analysis combines an acid hydrolysis, and then reduction and acetylation of the free sugars prior to GC analysis. Total acid hydrolysis was performed with 1M sulfuric acid (2 h or 6 h, 100° C.) after a pre- hydrolysis step with concentrated sulfuric acid 72% (30 min, 30° C.). The neutral sugar derivation method used follows that published by Blakeney et al. (1983). Alditol acetates were chromatographed on a DB 225 capillary column (J&W Scientific, Folsorn, CA, USA; temperature 205° C., carrier gas H2). Inositol was used as internal standard. Response factors were determined using standard sugars. Samples were determined in duplicate. Uronic acids in hydrolyzates were quantified using the metahydroxydiphenyl colorimetric acid method (Blumenkrantz & Asboe-Hansen, 1973).

TABLE C1
Glycosyl-linkage analysis of a pea fiber preparation (see Example 8 for a
description of the methodology)

| | Σ linked- | Σ linked- | | | | | | | | | |
|---|---|---|---|---|---|---|---|---|---|---|---|
| | sugars/ | sugars/ | Deduced | %/Σ sugars | | | | | | | |
| | DW | sugars | linkage | Rha | Fuc | Ara | Xyl | Gal | Glc | Man | UA |
| Rha | | 1.3% | Terminal | 0.1 | | | | | | | |
| | | | Rha(p) | | | | | | | | |
| | | | 2-Rha(p) | 0.4 | | | | | | | |
| | | | 2,4-Rha(p) | 0.8 | | | | | | | |
| Fuc | | ND | — | | ND | | | | | | |
| Ara | | 26.6% | Terminal Ara(f) | | | 9.4 | | | | | |
| | | | 5-Ara(f) | | | 12.2 | | | | | |
| | | | 2,5-Ara(f) | | | 3.5 | | | | | |
| | | | 3,5-Ara(f) | | | 1.4 | | | | | |
| Xyl | | 8.2% | Terminal Xyl(p) | | | | 1.3 | | | | |
| | | | 4-Xyl(p) | | | | 6.9 | | | | |
| Gal | | 5.1% | Terminal Gal(p) | | | | | 0.5 | | | |
| | | | 3-Gal(p) | | | | | 1.5 | | | |
| | | | 4-Gal(p) | | | | | 3.0 | | | |
| | | | 2,3,4-Gal(p) | | | | | 0.1 | | | |
| Glc | | 50.2% | Terminal Glc(p) | | | | | | 1.3 | | |
| | | | 4-Glc(p) | | | | | | 46.5 | | |
| | | | 3,4-Glc(p) | | | | | | 0.4 | | |
| | | | 4,6-Glc(p) | | | | | | 1.7 | | |
| | | | 2,3,4,6-Glc(p) | | | | | | 0.2 | | |
| Man | | ND | — | | | | | | | ND | |
| UA | | 8.4% | 4-GalA(p) | | | | | | | | 6.8 |
| | | | 4-GalA(p)- | | | | | | | | 0.7 |
| | | | methyl ester | | | | | | | | |
| | | | 3,4-GalA(p) | | | | | | | | 0.9 |
| | | | Calculated DM | | | | | | | | 8.0 |
| Total | 19.7% | | | | | | | | | | |
| linked- | | | | | | | | | | | |
| sugars/ | | | | | | | | | | | |
| DW | | | | | | | | | | | |

Rha = rhamnose,
Ara = arabinose,
Xyl = xylose,
Gal = galactose,
Glc = glucose,
Man = mannose,
UA = uronic acids,
ND = none detected,
DM = degree of methylation
Data are expressed in % of the total sugars identified (/Σ sugars). Yields of mass percentages are indicated in each table as % of total sugars identified per dried weight (%/DW).
Deduced linkage - 2018, Double reduction (Pettolino et al.)

TABLE C2
Glycosyl-linkage analysis of an extruded pea fiber preparation
(see Example 8 for a description of the methodology)

| | Σ linked- | Σ linked- | | | | | | | | | |
|---|---|---|---|---|---|---|---|---|---|---|---|
| | sugars/ | sugars/ | Deduced | %/Σ sugars | | | | | | | |
| | DW | sugars | linkage | Rha | Fuc | Ara | Xyl | Gal | Glc | Man | UA |
| Rha | | 1.0% | 2-Rha(p) | 0.5 | | | | | | | |
| | | | 2,4-Rha(p) | 0.5 | | | | | | | |
| Fuc | | ND | — | | ND | | | | | | |
| Ara | | 27.2% | Terminal Ara(f) | | | 13.3 | | | | | |
| | | | 5-Ara(f) | | | 10.4 | | | | | |
| | | | 2,5-Ara(f) | | | 2.8 | | | | | |
| | | | 3,5-Ara(f) | | | 0.7 | | | | | |
| Xyl | | 5.6% | Terminal Xyl(p) | | | | 2.8 | | | | |
| | | | 4-Xyl(p) | | | | 2.8 | | | | |
| Gal | | 3.2% | Terminal Gal(p) | | | | | 0.4 | | | |
| | | | 3-Gal(p) | | | | | 0.5 | | | |
| | | | 4-Gal(p) | | | | | 2.3 | | | |
| Glc | | 58.1% | Terminal Glc(p) | | | | | | 2.6 | | |
| | | | 4-Glc(p) | | | | | | 51.7 | | |
| | | | 3,4-Glc(p) | | | | | | 0.6 | | |
| | | | 4,6-Glc(p) | | | | | | 3.2 | | |
| Man | | ND | — | | | | | | | ND | |
| UA | | 5.0% | 4-GalA(p) | | | | | | | | 3.7 |
| | | | 4-GalA(p)- | | | | | | | | 0.5 |
| | | | methyl ester | | | | | | | | |
| | | | 3,4-GalA(p) | | | | | | | | 0.5 |
| | | | 3,4-GalA(p)- | | | | | | | | 0.2 |
| | | | methyl ester | | | | | | | | |
| | | | Calculated DM | | | | | | | | 14.2 |
| Total | 46.7% | | | | | | | | | | |
| linked- | | | | | | | | | | | |
| sugars/ | | | | | | | | | | | |
| DW | | | | | | | | | | | |

Rha = rhamnose,
Ara = arabinose,
Xyl = xylose,
Gal = galactose,
Glc = glucose,
Man = mannose,
UA = uronic acids,
ND = none detected,
DM = degree of methylation
Data are expressed in % of the total sugars identified (/Σ sugars). Yields of mass percentages are indicated in each table as % of total sugars identified per dried weight (%/DW).
Deduced linkage - 2018, Double reduction (Pettolino et al.)
Note:
the pea fiber preparation of Table C1 was extruded to obtain this preparation.

TABLE D
Glycosyl-linkage analysis of a citrus pectin preparation
(see Example 8 for a description of the methodology)

| | Σ linked- | Σ linked- | | | | | | | | | |
|---|---|---|---|---|---|---|---|---|---|---|---|
| | sugars/ | sugars/ | Deduced | %/Σ sugars | | | | | | | |
| | DW | sugars | linkage | Rha | Fuc | Ara | Xyl | Gal | Glc | Man | UA |
| Rha | | 1.5% | 2-Rha(p) | 1.2 | | | | | | | |
| | | | 2,4-Rha(p) | 0.3 | | | | | | | |
| Fuc | | ND | — | | ND | | | | | | |
| Ara | | 3.4% | Terminal Ara(f) | | | 0.8 | | | | | |
| | | | 5-Ara(f) | | | 1.2 | | | | | |
| | | | 2,5-Ara(f) | | | 0.2 | | | | | |
| | | | 3,5-Ara(f) | | | 1.2 | | | | | |
| Xyl | | ND | — | | | | ND | | | | |
| Gal | | 6.0% | 4-Gal(p) | | | | | 4.4 | | | |
| | | | 3,4-Gal | | | | | 0.1 | | | |
| | | | 4,6-Gal | | | | | 0.9 | | | |
| | | | 2,3,4-Gal | | | | | 0.7 | | | |
| Glc | | ND | — | | | | | | ND | | |
| Man | | ND | — | | | | | | | ND | |
| UA | | 88.6% | 4-GalA(p) | | | | | | | | 26.3 |
| | | | 4-GalA(p)- | | | | | | | | 60.8 |
| | | | methyl ester | | | | | | | | |
| | | | 2,4-GalA(p)- | | | | | | | | 0.5 |
| | | | methyl ester | | | | | | | | |
| | | | 3,4-GalA(p) | | | | | | | | 0.3 |
| | | | 3,4-GalA(p)- | | | | | | | | 0.8 |
| | | | methyl ester | | | | | | | | |
| | | | Calculated DM | | | | | | | | 70.1 |
| Total | 42.6% | | | | | | | | | | |
| linked- | | | | | | | | | | | |
| sugars/ | | | | | | | | | | | |
| DW | | | | | | | | | | | |

Rha = rhamnose,
Ara = arabinose,
Xyl = xylose,
Gal = galactose,
Glc = glucose,
Man = mannose,
UA = uronic acids,
ND = none detected,
DM = degree of methylation
Data are expressed in % of the total sugars identified (/Σ sugars). Yields of mass percentages are indicated in each table as % of total sugars identified per dried weight (%/DW).
Deduced linkage - 2018, Double reduction (Pettolino et al.)

TABLE E
Glycosyl-linkage analysis of a barley fiber preparation (see Example 8 for a description of the methodology)

| | Σ linked- | Σ linked- | | | | | | | | | | |
|---|---|---|---|---|---|---|---|---|---|---|---|---|
| | sugars/ | sugars/ | Deduced | %/Σ sugars | | | | | | | | |
| | DW | sugars | linkage | Rha | Fuc | Ara | Xyl | Gal | Glc | Man | UA | Hex |
| Rha | | ND | — | ND | | | | | | | | |
| Fuc | | ND | — | | ND | | | | | | | |
| Ara | | 1.6% | Terminal Ara(f) | | | 1.6 | | | | | | |
| Xyl | | 6.3% | 4-Xyl(p) | | | | 3.0 | | | | | |
| | | | 3,4-Xyl(p) | | | | 0.9 | | | | | |
| | | | 2,3,4-Xyl(p) | | | | 2.4 | | | | | |
| Gal | | 1.0% | 4-Gal(p) | | | | | 1.0 | | | | |
| Glc | | 84.5% | Terminal Glc(p) | | | | | | 3.0 | | | |
| | | | 3-Glc(p) | | | | | | 5.2 | | | |
| | | | 4-Glc(p) | | | | | | 71.3 | | | |
| | | | 4,6-Glc(p) | | | | | | 3.4 | | | |
| | | | 2,3,4,6-Glc(p) | | | | | | 1.5 | | | |
| Man | | ND | — | | | | | | | ND | | |
| UA | | ND | — | | | | | | | | ND | |
| Hex | | 6.6% | 2,4-Hex | | | | | | | | | 1.1 |
| | | | 3,4-Hex | | | | | | | | | 2.9 |
| | | | 2,3,4-Hex | | | | | | | | | 0.9 |
| | | | 3,4,6-Hex | | | | | | | | | 1.7 |
| Total | 18.2% | | | | | | | | | | | |
| linked- | | | | | | | | | | | | |
| sugars/ | | | | | | | | | | | | |
| DW | | | | | | | | | | | | |

Rha = rhamnose,
Ara = arabinose,
Xyl = xylose,
Gal = galactose,
Glc = glucose,
Man = mannose,
UA = uronic acids,
Hex = hexose,
ND = none detected,
DM = degree of methylation
Data are expressed in % of the total sugars identified (/Σ sugars). Yields of mass percentages are indicated in each table as % of total sugars identified per dried weight (%/DW).
Deduced linkage - 2018, Double reduction (Pettolino et al.)

TABLE F1
Glycosyl-linkage analysis of a citrus fiber preparation
(see Example 8 for a description of the methodology)

| | Σ linked- | Σ linked- | | | | | | | | | |
|---|---|---|---|---|---|---|---|---|---|---|---|
| | sugars/ | sugars/ | Deduced | %/Σ sugars | | | | | | | |
| | DW | sugars | linkage | Rha | Fuc | Ara | Xyl | Gal | Glc | Man | UA |
| Rha | | 0.9% | 2-Rha(p) | 0.9 | | | | | | | |
| Fuc | | ND | — | | ND | | | | | | |
| Ara | | 15.4% | Terminal Ara(f) | | | 2.4 | | | | | |
| | | | 5-Ara(f) | | | 8.3 | | | | | |
| | | | 3,5-Ara(f) | | | 4.7 | | | | | |
| Xyl | | 2.4% | 4-Xyl(p) | | | | 2.4 | | | | |
| Gal | | 10.3% | Terminal Gal(p) | | | | | 0.9 | | | |
| | | | 3-Gal(p) | | | | | 1.2 | | | |
| | | | 4-Gal(p) | | | | | 8.1 | | | |
| | | | 4,6-Gal(p) | | | | | 0.2 | | | |
| Glc | | 13.0% | 4-Glc(p) | | | | | | 13.0 | | |
| Man | | ND | — | | | | | | | ND | |
| UA | | 57.1% | Terminal | | | | | | | | 0.1 |
| | | | GalA(p) | | | | | | | | |
| | | | Terminal | | | | | | | | 0.4 |
| | | | GalA(p)-methyl | | | | | | | | |
| | | | ester | | | | | | | | |
| | | | 4-GalA(p) | | | | | | | | 24.0 |
| | | | 4-GalA(p)- | | | | | | | | 31.5 |
| | | | methyl ester | | | | | | | | |
| | | | 3,4-GalA(p) | | | | | | | | 0.4 |
| | | | 3,4-GalA(p)- | | | | | | | | 0.2 |
| | | | methyl ester | | | | | | | | |
| | | | 4,6-GalA(p) | | | | | | | | 0.1 |
| | | | 4,6-GalA(p)- | | | | | | | | 0.4 |
| | | | methyl ester | | | | | | | | |
| | | | Calculated DM | | | | | | | | 57.1 |
| Total | 9.5% | | | | | | | | | | |
| linked- | | | | | | | | | | | |
| sugars/ | | | | | | | | | | | |
| DW | | | | | | | | | | | |

Rha = rhamnose,
Ara = arabinose,
Xyl = xylose,
Gal = galactose,
Glc = glucose,
Man = mannose,
UA = uronic acids,
ND = none detected,
DM = degree of methylation
Data are expressed in % of the total sugars identified (/Σ sugars). Yields of mass percentages are indicated in each table as % of total sugars identified per dried weight (%/DW).
Deduced linkage - 2018, Double reduction (Pettolino et al.)

TABLE F2
Glycosyl-linkage analysis of an extruded citrus fiber preparation
(see Example 8 for a description of the methodology)

| | Σ linked- | Σ linked- | | | | | | | | | |
|---|---|---|---|---|---|---|---|---|---|---|---|
| | sugars/ | sugars/ | Deduced | %/Σ sugars | | | | | | | |
| | DW | sugars | linkage | Rha | Fuc | Ara | Xyl | Gal | Glc | Man | UA |
| Rha | | 0.7% | 2-Rha(p) | 0.7 | | | | | | | |
| Fuc | | ND | — | | ND | | | | | | |
| Ara | | 20.1% | Terminal Ara(f) | | | 8.8 | | | | | |
| | | | 5-Ara(f) | | | 8.5 | | | | | |
| | | | 3,5-Ara(f) | | | 2.8 | | | | | |
| Xyl | | 3.2% | 4-Xyl(p) | | | | 3.2 | | | | |
| Gal | | 5.1% | Terminal Gal(p) | | | | | 0.5 | | | |
| | | | 3-Gal(p) | | | | | 0.7 | | | |
| | | | 4-Gal(p) | | | | | 3.7 | | | |
| Glc | | 53.1% | Terminal Glc(p) | | | | | | 1.5 | | |
| | | | 4-Glc(p) | | | | | | 49.4 | | |
| | | | 3,4-Glc(p) | | | | | | 0.3 | | |
| | | | 4,6-Glc(p) | | | | | | 1.9 | | |
| | | | 2,3,4,6-Glc(p) | | | | | | 0.1 | | |
| Man | | 0.2% | 4,6-Man(p) | | | | | | | 0.2 | |
| UA | | 17.7% | 4-GalA(p) | | | | | | | | 6.3 |
| | | | 4-GalA(p)- | | | | | | | | 11.1 |
| | | | methyl ester | | | | | | | | |
| | | | Calculated DM | | | | | | | | 64.2 |
| Total | 18.7% | | | | | | | | | | |
| linked- | | | | | | | | | | | |
| sugars/ | | | | | | | | | | | |
| DW | | | | | | | | | | | |

Rha = rhamnose,
Ara = arabinose,
Xyl = xylose,
Gal = galactose,
Glc = glucose,
Man = mannose,
UA = uronic acids,
ND = none detected,
DM = degree of methylation
Data are expressed in % of the total sugars identified (/Σ sugars). Yields of mass percentages are indicated in each table as % of total sugars identified per dried weight (%/DW).
Deduced linkage - 2018, Double reduction (Pettolino et al.)
Note:
the citrus fiber preparation of Table F1 was extruded to obtain this preparation.

TABLE G

| Test Method | % | Pea Fiber | Citrus Fiber | Inulin | Barley Bran |
|---|---|---|---|---|---|
| CODEX 2011 | Total Dietary Fiber | 63.7-65.4 | 74.4-77.1 | 95.3-100 | 28.4-44.5 |
| | High Molecular Weight (Insoluble) | 61.0-63.1 | 37.2-41.7 | N.D. | 11.2-12.3 |
| | High Molecular weight (Soluble) | 1.2-1.6 | 30.6-36.2 | 75.3-76.8 | 15.9-31.4 |
| | Low Molecular Weight | 1.1-1.7 | 1.4-2.4 | 18.2-23.5 | 1.1-3.4 |
| Gravimetric | Cellulose | 16.2-18.2 | 15.6-21.1 | N.D. | 0.5 |
| Gravimetric | Lignin | 0.70-0.86 | 4.3-6.0 | 0.6-0.7 | 1.1 |
| | Free Sugars | | | | |
| AOAC 2018.16 | Fructose | N.D. | 1.8-1.9 | N.D. | 0.1 |
| | Galactose | N.D. | N.D. | N.D. | N.D. |
| | Glucose | N.D. | 1.6-2.6 | N.D. | 0.1 |
| | Sucrose | N.D. | 2.9-3.0 | N.D. | 1.0-1.9 |
| | Lactose | N.D. | 0.5 | N.D. | N.D. |
| | Isomaltulose | N.D. | N.D. | N.D. | N.D. |
| | Maltose | N.D. | N.D. | N.D. | 0.6-0.9 |
| | Total Sugars | | 6.8-7.4 | | 2.0-2.8 |
| | Bound Sugars | | | | |
| Acid Hydrolysis | Arabinose | 20.2-21.5 | 7.4-82 | N.D. | 2.8 |
| GC-FID* | Glucose | 16.8-21.6 | 4.5-5.0 | 3.4 | 65.1-66.9 |
| | Galactose | 2.9-3.3 | 3.6-4.7 | N.D. | 0.3-0.4 |
| | Xylose | 3.8 | 1.3-1.6 | 0.7 | 3.8 |
| | Mannose | N.D | 0.5-0.6 | N.D. | 0.6 |
| | Fucose | 0.2-0.3 | 0.3 | N.D. | N.D. |
| | Rhamnose | 0.7 | 1.1-1.3 | N.D. | N.D. |
| Polarimetric | Starch | 12.8-18.2 | N.D. | N.D. | 33.4-36.5 |
| AOAC 996.11 | Starch | 12.3-19.4 | 0.5-1.1 | N.D. | 32.7-33.7 |
| AOAC 995.16 | Beta Glucans | N.T. | N.T. | N.T. | 24.4-25.1 |
| HPAEC-PAD | Inulin | N.T. | N.T | 100 | N.T. |
| Kjeldahal | Protein | 7.91-10.6 | 5.7-7.5 | N.D. | 10.5-12.1 |
| Acid Hydrolysis | Fat | 0.86-0.94 | 2.0-3.0 | N.D. | 2.49-2.84 |
| | Particle Size (um) | | | | |
| Laser Diffraction | d10 | 9.3-11.8 | 59-107 | 10.1-12.7 | 78.3-130 |
| | d50 | 40.9-53.2 | 306-329 | 37.6-45.2 | 219-289 |
| | d90 | 110-128 | 686-712 | 122-125 | 470-544 |

*Fructose is unable to be detected with this methodology
N.D. = Not Detected
N.T. = Not Tested

### II. Food Compositions

In another aspect, the present disclosure provides food compositions comprising one or more fiber preparation, each fiber preparation independently selected from the group consisting of a barley fiber preparation, a citrus fiber preparation, a citrus pectin preparation, a high molecular weight inulin preparation, a pea fiber preparation, a sugar beet fiber preparation, and glycan equivalents thereof. The glycan equivalent can be a compositional glycan equivalent or a functional glycan equivalent. Food compositions encompassed by the present disclosure may contain 1, 2, 3, 4, 5, 6, 7, 8, 9, 10, 11, 12, 13 or more different fiber preparations independently selected from the above group. Suitable fiber preparations are described in detail in Section I. Typically, each fiber preparation alone, or a combination of fiber preparations, is in an amount that increases the fiber degrading capacity of gut microbiota in a subject and/or promotes a healthy gut microbiota in a subject when administered to the subject on a daily basis for at least 5 days (e.g., 5, 6, 7, 8, 9, 10, 11, 12, 13, 14 days or more). In some embodiments, a food composition is in a baked form. In some embodiments, a food composition is in an extruded or pressed form. Extruded foods can be shaped to limitless forms depending on the die. They also can be coated, filled, pressed into a bar (or other shape), or combinations thereof, with other ingredients using a binder. In some embodiments, a food composition is a bar; a drink; a gel, a gummy, a candy, or the like; a cookie, a cracker, a cake, or the like; a bread, a muffin, or the like; a dairy product (e.g., yogurt, ice cream or the like).

The term “wt % of the food composition” is the weight of an ingredient as a percentage of all ingredients in the food composition prior to processing (e.g., baking, extrusion, dehydration, etc.) into the final form (e.g., cookie, cracker, bar, extruded shape, gel, powder, etc.), but does not include any added water. Typically the ingredients are combined and then a suitable amount of water (e.g., about 15%) is added to make a dough for a baked product or a mix to go into an extrusion process. When combining the ingredients, all the ingredients may be added individually (inclusive of each fiber preparation), or various ingredients may be combined and then the combinations added. For instance, in some embodiments one or more fiber preparations may be first combined together to form a composition of fiber preparations, and then the composition of fiber preparations is combined with any other ingredients. In other embodiments, each fiber preparation may be added individually. The final moisture content of the baked, pressed or extruded product may vary, though typically the final moisture content may be around 2-5%, or more preferably 3%.

In some embodiments, the one or more fiber preparation, in total, is about 30 wt % to about 50 wt % of the food composition. In some embodiments, the one or more fiber preparation provides 50% of the food composition's total dietary fiber. In some embodiments, the one or more fiber preparation, in total, is about 30 wt % to about 50 wt % of the food composition and provides 50% of the food composition's total dietary fiber. In each of the above embodiments, the one or more fiber preparation, in total, may provide at least 3 g, at least 6 g, or at least 10 g of total dietary fiber per serving of the food composition. For instance, the one or more fiber preparations, in total, may provide 3 g, 4 g, 5 g, 6 g, 7 g, 8 g, 9 g, 10 g, or more of total dietary fiber per serving of the food composition. Serving size can vary, and may be about 20 g to about 50 g, about 25 g to about 40 g, about 30 g to about 40 g, or about 30 g to about 35 g. In some examples, the one or more fiber preparations, in total, may provide about 3 g to about 10 g of total dietary fiber per serving of the food composition. In other examples, the one or more fiber preparations, in total, may provide about 3 g to about 6 g of total dietary fiber per serving of the food composition, or about 6 g to about 10 g of total dietary fiber to the food composition. In preferred examples, the one or more fiber preparation comprises a pea fiber preparation and/or a glycan equivalent thereof, in particular, a pea fiber preparation of Section I and/or glycan equivalent.

As illustrated in the Examples, food compositions that differ in the number of barley fiber, citrus fiber, high molecular weight inulin and pea fiber preparations have overlapping and distinct effects on a subject's microbiome and on biomarkers of health, including biomarkers of cardiometabolic and immunoinflammatory state. Accordingly, the number of fiber preparations, and amounts of each, may be optimized to achieve a desired effect. Importantly, the Examples further illustrate the suitability of gnotobiotic mice colonized with gut microbial communities representing a human study population as a model system that can be used to select fiber preparations and define appropriate amounts. The Examples also identify health discriminatory biomarkers that can be measured in human blood samples that are linked to health discriminatory features of the gut microbiome (e.g., CAZymes, PULs, etc.). Thus, the Examples demonstrate that the gut mcirobiome may be used as a read-out to evaluate the effectiveness of a given food.

In a specific example, a food composition may comprise a first fiber preparation that is a pea fiber preparation or a glycan equivalent thereof, and a second fiber preparation that is a high molecular weight inulin preparation or a glycan equivalent thereof, wherein the first and second fiber preparation, in total, provide about 3 g to about 10 g of total dietary fiber per serving of the food composition. In another specific example, a food composition may comprise a first fiber preparation that is a pea fiber preparation or a glycan equivalent thereof, a second fiber preparation that is a high molecular weight inulin preparation or a glycan equivalent thereof, a third fiber preparation that is a citrus fiber preparation or a glycan equivalent thereof, and fourth fiber preparation that is a barley fiber preparation or a glycan equivalent thereof, wherein the first, second, third and fourth fiber preparation, in total, provide about 3 g to about 10 g of total dietary fiber per serving of the food composition. In further examples, the food compositions above may have amounts of each fiber preparation as indicated in the table below.

| | Citrus fiber | HMW inulin | Barley fiber |
|---|---|---|---|
| Pea or GE | or GE | or GE | or GE |
| about 25-40 | about 5-15 | about 30-40 | about 10-30 |
| wt % | wt % | wt % | wt % |
| about 30-40 | about 10-15 | about 30-40 | about 15-25 |
| wt % | wt % | wt % | wt % |
| about 60-70 | 0 wt % | 30-40 wt % | 0 wt % |
| wt % | | | |
| about 55-65 | 0 wt % | 35-45 wt % | 0 wt % |
| wt % | | | |
| about 60-65 | 0 wt % | 35-40 wt % | 0 wt % |
| wt % | | | |

GE = glycan equivalent
wt % = weight percentage, calculated as weight of individual fiber preparation/total weight of the four fiber preparations

In still further examples, the pea fiber preparation or glycan equivalent thereof may have a composition substantially similar to the pea fiber preparation of Table A or Table G, and/or a monosaccharide content substantially similar to the pea fiber preparation of Table B or Table G, and optionally glycosyl linkages substantially similar to the pea fiber preparation of Table C1 or C2. The high molecular weight inulin preparation or glycan equivalent thereof may have a composition substantially similar to the high molecular weight inulin preparation of Table A or Table G. The barley fiber preparation or glycan equivalent thereof may have a composition substantially similar to the barley fiber preparation of Table A or Table G, and/or a monosaccharide content substantially similar to the barley fiber preparation of Table B or Table G, and optionally glycosyl linkages substantially similar to the barley fiber preparation of Table E. The citrus fiber preparation or glycan equivalent thereof may have a composition substantially similar to the citrus fiber preparation of Table A or Table G, and/or a monosaccharide content substantially similar to the citrus fiber preparation of Table B or Table G, and optionally glycosyl linkages substantially similar to the citrus fiber preparation of Table F1 or F2.

In addition to the selection of fiber preparation(s) in a food composition and its amount, a food composition may be processed in a manner such that the food increases the fiber degrading capacity of a gut microbiota in a subject and/or promotes a healthy gut microbiota in a subject when administered to the subject on a daily basis for at least 5 days (e.g., at least 6 days, at least 7 days, etc.). In particular, whereby such food composition effects an increase in the total or relative abundance of Bacteroides species measured in a fecal sample obtained from a subject after the subject has consumed the food composition at least once a day for at least 5 days (e.g., at least 6 days, at least 7 days, etc.).

Food compositions may further comprise one or more additional food ingredient. These additional ingredients may contribute favorable organoleptic properties (e.g., taste, texture, etc.) to the food, improve the processing and handling of the food, contribute additional nutritional value to the food, and the like. Non-limiting examples of additional food ingredient include flours, meals, sweeteners, preservatives, color additives, flavors, spices, flavor enhancers, fats, oils, fat replacers (including components of formulations used to replace fats), nutrients, vitamins, minerals, emulsifiers, stabilizers, thickeners, binders, texturizers, pH control agents, leavening agents, anti-caking agents, humectants, firming agents, probiotics, postbiotics, and enzyme preparations, as well as fruits, vegetables and grains. Non-limiting examples of food ingredients are further detailed in Section II(i).

Example 11 illustrates how the selection of various forms of food and the use of additional food ingredients may influence organoleptic properties and/or nutritional values.

### (a) Exemplary Embodiments

Each of the following embodiments contains a plurality of fiber preparations. In order to accurately describe the amount of each fiber preparation in each embodiment, the plurality of fiber preparations is referred to as “a composition.” Use of the term “composition,” in regards to a plurality of fiber preparations in a food composition (both in this section and elsewhere in this disclosure), encompasses embodiments where the plurality of fiber preparations are combined as one composition which is then added to other food ingredients, embodiments where the plurality of fiber preparations are combined into more than one composition which are then added to other food ingredients, and embodiments where each fiber preparation is individually added to other food ingredients. This is consistent with the disclosures above stating fiber preparations may be added individually in the amounts described in this section.

In one embodiment, the present disclosure provides a pressed, extruded or baked food composition, wherein a 30 g serving of the food composition has at least 3 g or at least 6 g of total dietary fiber, and wherein the food composition comprises about 40 wt % to about 95 wt % of a composition of fiber preparations, the composition of fiber preparations comprising about 25 wt % to about 40 wt % of a pea fiber preparation or a glycan equivalent thereof, about 5 wt % to about 15 wt % of a citrus fiber preparation or a glycan equivalent thereof, about 30 wt % to about 40 wt % of a high molecular weight inulin preparation or a glycan equivalent thereof, and about 10 wt % to about 30 wt % of a barley fiber preparation or a glycan equivalent thereof.

In another embodiment, the present disclosure provides a pressed, extruded or baked food composition, wherein a 30 g serving of the food composition has at least 3 g or at least 6 g of total dietary fiber, and wherein the food composition comprises about 40 wt % to about 95 wt % of a composition of fiber preparations, the composition of fiber preparations comprising about 15 wt % to about 32 wt % of a sugar beet fiber preparation or a glycan equivalent thereof, about 5 wt % to about 15 wt % of a citrus fiber preparation or a glycan equivalent thereof, about 30 wt % to about 40 wt % of a high molecular weight inulin preparation or a glycan equivalent thereof, and about 10 wt % to about 30 wt % of a barley fiber preparation or a glycan equivalent thereof.

In another embodiment, the present disclosure provides a pressed, extruded or baked food composition, wherein a 30 g serving of the food composition has at least 3 g or at least 6 g of total dietary fiber, and wherein the food composition comprises about 40 wt % to about 95 wt % of a composition of fiber preparations, the composition of fiber preparations comprising 55 wt % to about 65 wt % of one or more pea fiber preparation or a glycan equivalent thereof and about 30 wt % to about 40 wt % of one or more high molecular weight inulin preparation or a glycan equivalent thereof.

In another embodiment, the present disclosure provides a pressed, extruded or baked food composition, wherein a 30 g serving of the food composition has at least 3 g or at least 6 g of total dietary fiber, and wherein the food composition comprises about 40 wt % to about 95 wt % of a composition of fiber preparations, the composition of fiber preparations comprising about 60 wt % to about 65 wt % of one or more pea fiber preparation or a glycan equivalent thereof and about 30 wt % to about 35 wt % of one or more high molecular weight inulin preparation or a glycan equivalent thereof.

In another embodiment, the present disclosure provides a pressed, extruded or baked food composition, wherein a 30 g serving of the food composition has at least 3 g or at least 6 g of total dietary fiber, and wherein the food composition comprises about 40 wt % to about 95 wt % of a composition of fiber preparations, the composition of fiber comprising of about 55 wt % to about 65 wt % of one or more sugar beet fiber preparation or a glycan equivalent thereof and about 30 wt % to about 40 wt % of one or more high molecular weight inulin preparation or a glycan equivalent thereof.

In another embodiment, the present disclosure provides a pressed, extruded or baked food composition, wherein a 30 g serving of the food composition has at least 3 g or at least 6 g of total dietary fiber, and wherein the food composition comprises about 40 wt % to about 95 wt % of a composition of fiber preparations, the composition of fiber preparations comprising of about 45 wt % to about 55 wt % of one or more sugar beet preparation or a glycan equivalent thereof and about 30 wt % to about 50 wt % of one or more high molecular weight inulin preparation or a glycan equivalent thereof.

In another embodiment, the present disclosure provides a pressed, extruded or baked food composition, wherein a 30 g serving of the food composition has at least 3 g or at least 6 g of total dietary fiber, and wherein the food composition comprises about 40 wt % to about 95 wt % of a composition of fiber preparations, the composition of fiber preparations comprising 25 wt % to about 40 wt % of a pea fiber preparation or a glycan equivalent thereof, about 5 wt % to about 15 wt % of a citrus fiber preparation or a glycan equivalent thereof, about 30 wt % to about 40 wt % of a high molecular weight inulin preparation or a glycan equivalent thereof, and about 10 wt % to about 30 wt % of a barley fiber preparation or a glycan equivalent thereof.

In another embodiment, the present disclosure provides a pressed, extruded or baked food composition, wherein a 30 g serving of the food composition has at least 3 g or at least 6 g of total dietary fiber, and wherein the food composition comprises about 40 wt % to about 95 wt % of a composition of fiber preparations, the composition of fiber preparations consisting essentially of about 15 wt % to about 32 wt % of a sugar beet fiber preparation or a glycan equivalent thereof, about 5 wt % to about 15 wt % of a citrus fiber preparation or a glycan equivalent thereof, about 30 wt % to about 40 wt % of a high molecular weight inulin preparation or a glycan equivalent thereof, and about 10 wt % to about 30 wt % of a barley fiber preparation or a glycan equivalent thereof.

In another embodiment, the present disclosure provides a pressed, extruded or baked food composition, wherein a 30 g serving of the food composition has at least 6 g of total dietary fiber, and wherein the food composition comprises about 40 wt % to about 95 wt % of a composition of fiber preparations, the composition of fiber preparations consisting essentially of about 55 wt % to about 65 wt % of one or more pea fiber preparation or a glycan equivalent thereof and about 30 wt % to about 40 wt % of one or more high molecular weight inulin preparation or a glycan equivalent thereof.

In another embodiment, the present disclosure provides a pressed, extruded or baked food composition, wherein a 30 g serving of the food composition has at least 6 g of total dietary fiber, and wherein the food composition comprises about 40 wt % to about 95 wt % of a composition of fiber preparations, the composition of fiber preparations consisting essentially of about 60 wt % to about 65 wt % of one or more pea fiber preparation or a glycan equivalent thereof and about 30 wt % to about 35 wt % of one or more high molecular weight inulin preparation or a glycan equivalent thereof.

In another embodiment, the present disclosure provides a pressed, extruded or baked food composition, wherein a 30 g serving of the food composition has at least 6 g of total dietary fiber, and wherein the food composition about 40 wt % to about 95 wt % of a composition of fiber preparations, the composition of fiber preparations consisting essentially of about 55 wt % to about 65 wt % of one or more sugar beet fiber preparation or a glycan equivalent thereof and about 30 wt % to about 40 wt % of one or more high molecular weight inulin preparation or a glycan equivalent thereof.

In another embodiment, the present disclosure provides a pressed, extruded or baked food composition, wherein a 30 g serving of the food composition has at least 6 g of total dietary fiber, and wherein the food composition about 40 wt % to about 95 wt % of a composition of fiber preparations, the composition of fiber preparations consisting essentially of about 45 wt % to about 55 wt % of one or more sugar beet preparation or a glycan equivalent thereof and about 30 wt % to about 50 wt % of one or more high molecular weight inulin preparation or a glycan equivalent thereof.

In another embodiment, the present disclosure provides a pressed, extruded or baked food composition, wherein a 30 g serving of the food composition has at least 6 g of total dietary fiber and wherein the food composition about 40 wt % to about 95 wt % of a composition of fiber preparations, the composition of fiber preparations consisting essentially of about 45 wt % to about 55 wt % of one or more sugar beet preparation or a glycan equivalent thereof and about 30 wt % to about 50 wt % of one or more high molecular weight inulin preparation or a glycan equivalent thereof.

In some of the above embodiments, there may be about 30-40 wt % of one or more pea fiber preparation or a glycan equivalent thereof, about 9-11 wt % of one or more citrus fiber preparation or a glycan equivalent thereof, about 30-40 wt % of a high molecular weight inulin preparation or a glycan equivalent thereof, and about 18-22 wt % of a barley fiber preparation or a glycan equivalent thereof, in the composition of fiber preparations. In another example, there may be about 30-35 wt % of one or more pea fiber preparation or a glycan equivalent thereof, about 9-11 wt % of one or more citrus fiber preparation or a glycan equivalent thereof, about 35-40 wt % of one or more high molecular weight inulin preparation or a glycan equivalent thereof, and about 18-22 wt % of one or more barley fiber preparation or a glycan equivalent thereof, in the composition of fiber preparations. In still another example, there may be about 33 wt % of one or more pea fiber preparation or a glycan equivalent thereof, about 11 wt % of one or more citrus fiber preparation or a glycan equivalent thereof, about 36 wt % of one or more high molecular weight inulin preparation or a glycan equivalent thereof, and about 20 wt % of one or more barley fiber preparation or a glycan equivalent thereof, in the composition of fiber preparations.

In some of the above embodiments, there may be about 60 wt % to about 65 wt % of one more pea fiber preparation and about 35 wt % to about 40 wt % of one or more high molecular weight inulin preparation, in the composition of fiber preparations. In further embodiments, there may be about 65 wt % of one more pea fiber preparation and about 35 wt % of one or more high molecular weight inulin preparation, in the composition of fiber preparations.

In some of the above embodiments, there may be about 50 wt % to about 55 wt % of one more pea fiber preparation and about 35 wt % to about 40 wt % of one or more high molecular weight inulin preparation, in the composition of fiber preparations. In further embodiments, there may be about 55 wt % of one more pea fiber preparation and about 45 wt % of one or more high molecular weight inulin preparation, in the composition of fiber preparations.

In further embodiments, the composition of fiber preparation contains only one type of each fiber preparation. For instance, there may be about 55 wt % of one pea fiber preparation and about 45 wt % of one high molecular weight inulin preparation, in the composition of fiber preparations.

Suitable barley fiber preparations, citrus fiber preparations, citrus pectin preparations, high molecular weight inulin preparations, pea fiber preparations, and sugar beet fiber preparations are described above in Section I, as are compositional glycan equivalents and functional glycan equivalents of barley fiber preparations, citrus fiber preparations, citrus pectin preparations, high molecular weight inulin preparations, pea fiber preparations, and sugar beet fiber preparations. As non-limiting examples, the pea fiber preparation may have a composition substantially similar to the pea fiber preparation of Table A or Table G, and/or a monosaccharide content substantially similar to the pea fiber preparation of Table B or Table G, and optionally glycosyl linkages substantially similar to the pea fiber preparation of Table C1 or C2; the high molecular weight inulin preparation may have a composition substantially similar to the high molecular weight inulin preparation of Table A or Table G; the barley fiber preparation may have a composition substantially similar to the barley fiber preparation of Table A or Table G, and/or a monosaccharide content substantially similar to the barley fiber preparation of Table B or Table G, and optionally glycosyl linkages substantially similar to the barley fiber preparation of Table E; the citrus fiber preparation may have a composition substantially similar to the citrus fiber preparation of Table A or Table G, and/or a monosaccharide content substantially similar to the citrus fiber preparation of Table B or Table G, and optionally glycosyl linkages substantially similar to the citrus fiber preparation of Table F1 or F2.

When the food composition is a pressed or extruded food composition in the above embodiments, the composition of fiber preparations may comprise about 40 wt % to about 95 wt %, about 50 wt % to about 90 wt %, or about 60 wt % to about 80 wt % of the food. Alternatively, the composition of fiber preparations may comprise about 40 wt % to about 80 wt %, about 40 wt % to about 70 wt %, or about 40 wt % to about 60 wt % of the food composition. In still another alternative, the composition of fiber preparations may comprise about 40 wt % to about 50 wt % of the food composition.

When the food composition is a baked food composition in the above embodiments, the composition of fiber preparations may comprise about 40 wt % to about 60 wt %, about 40 wt % to about 50 wt %, or about 50 wt % to about 60 wt % of the food composition. In still another alternative, the composition of fiber preparations may comprise about 40 wt % to about 50 wt % of the food composition.

In each of the above embodiments, the composition of fiber preparations may provide about 90% or more of the total dietary fiber in the food composition. For instance, the composition of fiber preparations may provide about 90%, about 91%, about 92%, about 93%, about 94%, about 95%, about 96%, about 97%, about 98%, about 99%, or about 100% of the total dietary fiber in the food composition. In some embodiments, the composition of fiber preparations may provide about 95% or more of the total dietary fiber in the food composition. In some embodiments, the composition of fiber preparations may provide about 98% or more of the total dietary fiber in the food composition.

In each of the above embodiments, the baked, pressed or extruded food composition may further comprise one or more additional ingredient including, not limited to, flours, meals, sweeteners, preservatives, color additives, flavors, spices, flavor enhancers, fats, oils, fat replacers (including components of formulations used to replace fats), nutrients, vitamins, minerals, emulsifiers, stabilizers, thickeners, binders, texturizers, pH control agents, leavening agents, anti-caking agents, humectants, firming agents, and enzyme preparations. These additional ingredients may contribute favorable organoleptic properties (e.g., taste, texture, etc.) to the food and/or improve the processing and handling of the food.

In a specific embodiment, a baked, pressed or extruded food has a composition shown in Table H or Table I.

TABLE H

| INGREDIENTS | Composition 1 | | Composition 2 | | Composition 3 | |
|---|---|---|---|---|---|---|
| Flours: Wheat, Rice, | 0-60 | wt % | 14.4 | wt % | 27.9 | wt % |
| Corn or a blend | | | | | | |
| Sugar | 0-30 | wt % | 5 | wt % | 5 | wt % |
| Rice Starch | 0-40 | wt % | 30 | wt % | 20 | wt % |
| (waxy variety) | | | | | | |
| Salt | 0-5 | wt % | 0.5 | wt % | 0.5 | wt % |
| Sodium Bicarbonate or | 0-5 | wt % | 1 | wt % | 1 | wt % |
| Calcium carbonate | | | | | | |
| GMS (emulsifier) | 0-2 | wt % | 0.2 | wt % | 0.2 | wt % |
| Composition of Fiber | 40-95 | wt % | 49.0 | wt % | 45.5 | wt % |
| Preparations | | | | | | |

| | | | |
|---|---|---|---|
| TOTAL | 100 | 100 | 100 |

TABLE I

| | Composition | | | | | | | |
|---|---|---|---|---|---|---|---|---|
| INGREDIENTS | 1 | | 2 | | 3 | | 4 | |
| Flours: Wheat, Rice, | 14.4 | wt % | 27.9 | wt % | 23.4 | wt % | 36.9 | wt % |
| Corn or a blend | | | | | | | | |
| Sugar | 5 | wt % | 5 | wt % | 5 | wt % | 5 | wt % |
| Rice Starch (waxy variety) | 30 | wt % | 20 | wt % | 30 | wt % | 20 | wt % |
| Salt | 0.5 | wt % | 0.5 | wt % | 0.5 | wt % | 0.5 | wt % |
| Sodium Bicarbonate | 1 | wt % | 1 | wt % | 1 | wt % | 1 | wt % |
| or Calcium carbonate | | | | | | | | |
| GMS (emulsifier) | 0.2 | wt % | 0.2 | wt % | 0.2 | wt % | 0.2 | wt % |
| Pea Fiber Preparation | 16.2 | wt % | 29.1 | wt % | 0 | wt % | 0 | wt % |
| Citrus Fiber Preparation | 5.4 | wt % | 0 | wt % | 5.4 | wt % | 0 | wt % |
| Inulin, HMW Preparation | 17.6 | wt % | 16.4 | wt % | 17.6 | wt % | 16.4 | wt % |
| Barley Fiber Preparation | 9.8 | wt % | 0 | wt % | 9.8 | wt % | 0 | wt % |
| Sugar Beet Fiber | 0 | wt % | 0 | wt % | 7.2 | wt % | 20.1 | wt % |
| Preparation | | | | | | | | |

| | | | | |
|---|---|---|---|---|
| TOTAL | 100 | 100 | 100 | 100 |

### (b) Increases the Fiber Degrading Capacity and/or Promotes a Healthy Gut Microbiota

The “fiber degrading capacity” of a subject's gut microbiota is defined by its compositional state, specifically the absence, presence and abundance of primary and secondary consumers of dietary fiber. Microbes that are primary consumers initiate degradation of dietary fibers, while secondary consumers utilize glycans that are released by primary consumers. Increasing the fiber degrading capacity of a subject's gut microbiota may include, for example and without limitation, effecting an increase in the total and/or relative abundance of microorganisms with polysaccharide utilization loci (PULs) and/or genomic loci encoding CAZymes measured in a fecal sample obtained from a subject after the subject has consumed the food composition at least once a day for at least 5 days (e.g., at least 6 days, at least 7 days, etc.). In another example, increasing the fiber degrading capacity of a subject's gut microbiota may effect an increase in the total and/or relative abundance of a subset (one or more) of microorganisms with polysaccharide utilization loci (PULs) and/or genomic loci encoding CAZymes measured in a fecal sample obtained from a subject after the subject has consumed the food composition at least once a day for at least 5 days (e.g., at least 6 days, at least 7 days, etc.), the subset of microorganisms chosen from Bacteroides ovatus, Bacteroides cellulosilyticus, Bacteroides thetaiotaomicron, Bacteroides vulgatus, Bacteroides caccae, Bacteroides finegoldfi, Bacteroides massiliensis, Collinsella aerofaciens, Escherichia coli, Odoribacter splanchnicus, Parabacteroides distasonis, a Ruminococcaceae sp., or Subdoligranulum variabile. In another example, increasing the fiber degrading capacity of a subject's gut microbiota may effect an increase in the total or relative abundance of Bacteroides species measured in a fecal sample obtained from a subject after the subject has consumed the food composition at least once a day for at least 5 days (e.g., at least 6 days, at least 7 days, etc.). In another example, increasing the fiber degrading capacity of a subject's gut microbiota may effect an increase in the total or relative abundance of a subset (one or more) of Bacteroides species measured in a fecal sample obtained from a subject after the subject has consumed the food composition at least once a day for at least 5 days (e.g., at least 6 days, at least 7 days, etc.), the subset of Bacteroides species chosen from B. caccae, B. cellulosilyticus, B. finegoldfi, B. massiliensis, B. ovatus, B. thetaiotaomicron, or B. vulgatus. Alternatively or in addition, increasing the fiber degrading capacity of a subject's gut microbiota may include effecting an increase in the abundance or activity of one or more protein encoded by a PUL (with or without concomitant changes in microorganism abundance) and/or one or more CAZyme. In some examples, the one or more protein with an increased abundance or activity has α-L-arabinofuranosidase, β-galactosidase, N-acetylmuramidase, or endo-1,2,-α-mannanase enzymatic activities. In the above examples, the PULs may be chosen from PUL5, PUL6, PUL7, PUL27, PUL31, PUL34, PUL35, PUL38, PUL42, PUL43, PUL73, PUL75, PUL83, and PUL97, and/or the one or CAZymes may be chosen from GH5_1, GH5_4, GH5_5, GH5_46, GH43_1, GH43_2, GH43_3, GH43_8, GH43_9, GH43_12, GH43_16, GH43_17, GH43_18, GH43_19, GH43_28, GH43_29, GH43_31, GH43_33, GH43_34, GH43_35, GH43_38, GH99, GH108, GH116, and GH147.

In some embodiments, administration of a food composition described in this section, at least once daily for a minimum of five days, to a subject, increases the representation of members of one or more CAZyme family measured in a fecal sample obtained from the subject, wherein the one or more CAZyme family is selected from the group consisting of GH5_1, GH5_4, GH5_5, GH5_46, GH43_1, GH43_2, GH43_3, GH43_8, GH43_9, GH43_12, GH43_16, GH43_17, GH43_18, GH43_19, GH43_28, GH43_29, GH43_31, GH43_33, GH43_34, GH43_35, GH43_38, GH99, GH108, GH116, and GH147. In further embodiments, the one or more CAZyme family is selected from GH43_33, GH147, GH108, and GH99. As detailed in the Examples, increased representation of members of a CAZyme family may be an increase in genes encoding members of a CAZyme family. Increased representation of a CAZyme family may also be an increase in the abundance or activity of proteins in a CAZyme family. Methods for measuring protein abundance and enzyme activity are known in the art. Increasing the representation of one or more of these CAZyme families has a beneficial effect on or more aspects of a subject's health including but not limited to gut microbiota health, weight management, chronic inflammation, cardiovascular health, satiety, and glucose metabolism. In some examples, the subject is a healthy subject. In some examples, the subject is overweight or obese (e.g., as defined by a BMI outside the normal range for the subject's age, sex, and/or ethnicity). In some examples, the subject typically consumes a diet low in total dietary fiber (e.g., less than about 25 g per day). In some examples, the subject typically consumes a Western diet. A “Western diet” refers to a diet high in red meat, dairy products, processed and artificially sweetened foods and/or drinks, and salt, with minimal intake of fruits, vegetables, fish, legumes, and whole grains. An exemplary Western diet is the HiSF/LoFV diet detailed in the examples that is suitable for animals), and human equivalents thereof.

To “promote a healthy gut microbiota in a subject” means to change the feature of the microbiota or microbiome of the subject with the unhealthy gut microbiota in a manner towards the healthy subjects, and encompasses complete repair (i.e., the measure of gut microbiota health does not deviate by 1.5 standard deviation or more) and levels of repair that are less than complete. This may include, for example and without limitation, effecting an increase in the total abundance of Bacteroides species measured in a fecal sample obtained from a subject after the subject has consumed the food at least once a day for 5 days (e.g., at least 6 days, at least 7 days, etc.). Promoting a healthy gut microbiota in a subject also includes preventing the development of an unhealthy gut microbiota in a subject. In preferred embodiments, the microbiota of a subject is changed with regards to relative abundances of microbial community members and/or expression of proteins encoded by PULs, for instance as detailed in the Examples.

In still further embodiments, food compositions of the present disclosure have a beneficial effect on a subject's health after the subject has consumed the food composition for at least once a day for at least 5 days, or at least 7 days. For instance, administration for 5, 6, 7, 8, 9, 10, 11, 12, 13, 14 or more days may result in a beneficial effect. The improved aspect of the subject's health may be an improvement in weight management, chronic inflammation, cardiovascular health, satiety, and/or glucose metabolism. Non-limiting examples of measurable improvements in weight management may be a reduction in total body weight, a reduction in BMI, a reduction in weight gain, a reduction in fat mass gain, an increase in lean mass, a decrease in waist circumference, a decrease in waist to hip ratio, an increase in adiponectin levels, an increase in leptin levels, a decrease in resistin levels, or any combination thereof. Non-limiting examples of measurable improvements in chronic inflammation include a decrease in one or more plasma protein selected from CCL3, CRP, SPP1, F2, F3, VEGFA, PDGFRB, EFNA5, EPHA1, EPHA2, IL-6, IL-8, IL-1b, IL-1R1, IL-12, IL-17, IL-18, TNF-α, NF-kB, IFN-γ, and ceramides. Non-limiting examples of measurable improvements in cardiovascular health include a decrease in one or more plasma protein selected from C3, C1R, C4A/C4B, F3, SERPINE1, MASP1, PDGFRA, ICAM-1, VCAM-1, MCP-1, PAI-1, P-selectin, thromboxane-A2, F2a-isoprostanes, TBARS, MDA; as well as changes in LDL-cholesterol, HDL-cholesterol, total cholesterol, oxidized LDL, triglycerides, platelet aggregation and blood clotting. Non-limiting examples of measurable improvements in glucose metabolism include changes in fasting glucose, postprandial glucose, fasting insulin, postrprandial glucose, HOMAIR, HbA1c, glycated albumin, fructosamine, glucagon, QIUCKI, ISI, GIP, and GLP-1. Non-limiting examples of measurable improvements in satiety include improvements in AGRP, appetite VAS scores, food intake, GLP-1, PYY, GIP, ghrelin, cholecystokinin and leptin. In some examples, the improved aspect of the subject's health may be a reduction in total body weight, a reduction in BMI, a reduction in weight gain, a reduction in fat mass gain, an increase in fecal levels of succinate, a decrease in serum cholesterol, an increase in insulin sensitivity, a decrease in plasma markers of inflammation, an improvement in the relative abundances of health discriminatory plasma proteins, and/or an improvement in biomarkers/mediators of gut barrier function.

### III. Bioactive Glycans

Applicants have identified fiber preparations that promote a healthy gut microbiota in a subject, and further discovered that each fiber preparation has a number of bioactive glycans responsible for the observed beneficial effect(s). Thus, in another aspect, the present disclosure provides a composition comprising an enriched amount of one or more bioactive glycan, wherein “an enriched amount” refers to an amount of a bioactive glycan that is more than is found in a naturally occurring plant or plant part, and more than is found in commercially available fiber preparations, such as those used in Examples 2-6. A composition comprising an enriched amount of a bioactive glycan may be a purified (partially or completely) fraction from a commercially available fiber preparation. Alternatively, a composition comprising an enriched amount of a bioactive glycan may comprise a chemically synthesized version of the bioactive glycan. The bioactive glycan may be enriched by about 10 wt % wt to about 50 wt %, about 50 wt % to about 100 wt % or more. For instance, the bioactive glycan may be enriched by about 2-fold, about 3-fold, about 4-fold, about 5-fold, about 6-fold, about 7-fold, about 8-fold, about 9-fold, about 10-fold or more. In another example, the bioactive glycan may be enriched by about 20-fold, about 30-fold, about 40-fold, about 50-fold, about 60-fold, about 70-fold, about 80-fold, about 90-fold, about 100-fold or more. In another example, the bioactive glycan may be enriched by about 500-fold, 1000-fold, or more.

Bioactive glycans of barley fiber, citrus fiber, citrus pectin, high molecular weight inulin, pea fiber, and sugar beet fiber can be identified as detailed herein. For instance, pea fiber includes one or more bioactive arabinan of formula (I)

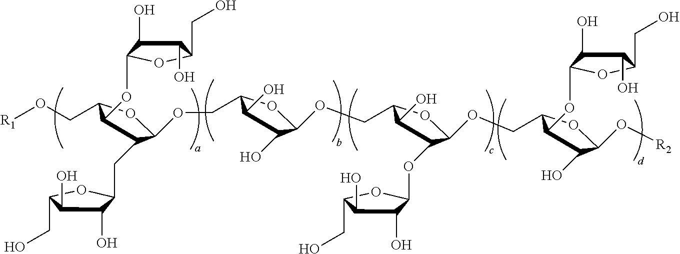

wherein a is about 0.1 to about 0.3, b is about 0.4 to about 0.6, c is about 0.1 to about 0.4, d is about 0.04 to about 0.06 (calculated from the fractional abundance of arabinose linkages where the arabinose contained a 5-linkage, as determined by partially methylated alditol acetate GC-MS analysis); wherein R₁ and R₂ are each independently selected from H, a glycosyl, a sugar moiety (modified or not), an oligosaccharide (branched or not), or a polysaccharide (branched or not), and a polysaccharide containing galacturonic acid, galactose, and rhamnose. Example 10 describes methods for obtaining a composition that is enriched for this bioactive arabinan; however, alternative purification methods may also be used. Alternatively, a chemically synthesized version may be used. An approach similar to the one detailed in Example 10 may be used to identify bioactive glycans in barley fiber, citrus fiber, citrus pectin, high molecular weight inulin, and sugar beet fiber.

The present disclosure also provides food compositions comprising a composition of this section. The food composition may further comprise one or more additional food ingredient including, not limited to, flours, meals, sweeteners, preservatives, color additives, flavors, spices, flavor enhancers, fats, oils, fat replacers (including components of formulations used to replace fats), nutrients, vitamins, minerals, emulsifiers, stabilizers, thickeners, binders, texturizers, pH control agents, leavening agents, anti-caking agents, humectants, firming agents, probiotics, and enzyme preparations.

The amount of a composition of this section in a food composition may vary. In some embodiments, a composition may be about 40 wt % to about 60 wt % of the ingredients used to make the food composition (excluding any added water). In some embodiments, a composition may be about 45 wt % to about 50 wt % of the ingredients used to make the food composition (excluding any added water).

In certain embodiments, the composition provides about 90% or more of the total dietary fiber in the food composition. For instance, the composition may provide about 90%, about 91%, about 92%, about 93%, about 94%, about 95%, about 96%, about 97%, about 98%, about 99%, or about 100% of the dietary fiber in the food composition. In one example, the composition provides about 95% or more of the total dietary fiber in the food composition. In another example, the composition provides about 98% or more of the total dietary fiber in the food composition.

In further embodiments, the food composition provides at least 6 g of dietary fiber per serving. In some examples, the food composition may provide at least 7 grams, at least 8 grams, at least 9 grams, or at least 10 grams of dietary fiber per serving. In other examples, the food composition may provide about 6 g to about 20 g, about 6 g to about 15 g, or about 6 g to about 10 g of dietary fiber per serving.

In some embodiments, a food composition is in a baked form. In some embodiments, a food composition is in a pressed or extruded form. In some embodiments, a food is in a powder form to be reconstituted. In some embodiments, a food is a bar; a drink; a gel, a gummy, a candy or the like; a cookie, a cracker, a cake, or the like; a dairy product (e.g., yogurt, ice cream or the like).

The present disclosure also provides other oral dosage forms comprising a composition of this section. Suitable dosage forms include a tablet, including a suspension tablet, a chewable tablet, an effervescent tablet or caplet; a pill; a powder such as a sterile packaged powder, a dispensable powder, and an effervescent powder; a capsule including both soft or hard gelatin capsules such as HPMC capsules; a lozenge; pellets; granules; liquids; suspensions; emulsions; or semisolids and gels. Capsule and tablet formulations may include, but are not limited to binders, lubricants, and diluents. Capsules and tablets may be coated according to methods well known in the art. Aqueous suspension formulations may include but are not limited to dispersants, flavor-modifying agents, taste-masking agents, and coloring agents.

### IV. Methods

In another aspect, the present disclosure provides methods for increasing the fiber degrading capacity of a subject's gut microbiota, promoting a healthy gut microbiota in a subject and/or improving a subject's health, the method comprising orally administering to a subject at least 3 grams or at least 6 grams of total dietary fiber per day in the form of a composition of Section I or Section III, or a food composition of Section I, II, or III. At least 3 grams of total dietary fiber per day includes 3 grams, 4 grams, 5 grams, 6 grams, 7 grams, or more of total dietary fiber per day. At least 6 grams of total dietary fiber per day includes 6 grams, 7 grams, 8 grams, 9 grams, 10 grams, 11 grams, 12 grams, 13 grams, 14, grams, 15 grams or more of total dietary fiber per day. In some embodiments, the method comprises orally administering to a subject at least 7 grams, at least 8 grams, at least 9 grams, or at least 10 grams of total dietary fiber per day in the form of a composition of Section I or Section III, or a food composition of Section I, II, or III. In some embodiments, the method comprises orally administering to a subject about 6 grams to about 10 grams of total dietary fiber per day in the form of a composition of Section I or Section III, or a food composition of Section I, II, or III. If the composition of Section I or Section III does not contain at least 6 g of total dietary fiber, multiple doses of the composition can be administered. Similarly, the number of servings of the food composition of Section I, II, or III can be adjusted such that at least 6 g of dietary fiber is consumed by the subject.

In some examples, increasing the fiber degrading capacity of a subject's gut microbiota may include effecting an increase in the total and/or relative abundance of microorganisms with polysaccharide utilization loci (PULs) measured in a fecal sample obtained from a subject after the subject has consumed at least 3 grams or at least 6 grams of total dietary fiber per day in the form of a composition of Section I or Section III, or a food composition of Section I, II, or III. In another example, increasing the fiber degrading capacity of a subject's gut microbiota may effect an increase in the total and/or relative abundance of a subset (one or more) of microorganisms with polysaccharide utilization loci (PULs) measured in a fecal sample obtained from a subject after the subject has consumed at least 3 grams or at least 6 grams of total dietary fiber per day in the form of a composition of Section I or Section III, or a food composition of Section I, II, or III, the subset of microorganisms chosen from Bacteroides ovatus, Bacteroides cellulosilyticus, Bacteroides thetaiotaomicron, Bacteroides vulgatus, Bacteroides caccae, Bacteroides finegoldfi, Bacteroides massiliensis, Collinsella aerofaciens, Escherichia coli, Odoribacter splanchnicus, Parabacteroides distasonis, a Ruminococcaceae sp., or Subdoligranulum variabile. In another example, increasing the fiber degrading capacity of a subject's gut microbiota may effect an increase in the total or relative abundance of Bacteroides species measured in a fecal sample obtained from a subject after the subject has consumed at least 3 grams or at least 6 grams of total dietary fiber per day in the form of a composition of Section I or Section III, or a food composition of Section I, II, or III. In another example, increasing the fiber degrading capacity of a subject's gut microbiota may effect an increase in the total or relative abundance of a subset (one or more) of Bacteroides species measured in a fecal sample obtained from a subject after the subject has consumed at least 3 grams or at least 6 grams of total dietary fiber per day in the form of a composition of Section I or Section III, or a food composition of Section I, II, or III, the subset of Bacteroides species chosen from B. caccae, B. cellulosilyticus, B. finegoldfi, B. massiliensis, B. ovatus, B. thetaiotaomicron, or B. vulgatus. Alternatively or in addition, increasing the fiber degrading capacity of a subject's gut microbiota may include effecting an increase in the abundance or activity of one or more protein encoded by a PUL (with or without concomitant changes in microorganism abundance). In some examples, the one or more protein with an increased abundance or activity has α-L-arabinofuranosidase, β-galactosidase, N-acetylmuramidase, or endo-1,2,-α-mannanase enzymatic activities. In the above examples, the PULs are chosen from PUL5, PUL6, PUL7, PUL27, PUL31, PUL34, PUL35, PUL38, PUL42, PUL43, PUL73, PUL75, PUL83, and PUL97, and/or the one or CAZymes may be chosen from GH5_1, GH5_4, GH5_5, GH5_46, GH43_1, GH43_2, GH43_3, GH43_8, GH43_9, GH43_12, GH43_16, GH43_17, GH43_18, GH43_19, GH43_28, GH43_29, GH43_31, GH43_33, GH43_34, GH43_35, GH43_38, GH99, GH108, GH116, and GH147.

To “promote a healthy gut microbiota in a subject” means to change the feature of the microbiota or microbiome of the subject with the unhealthy gut microbiota in a manner towards the healthy subjects, and encompasses complete repair (i.e., the measure of gut microbiota health does not deviate by 1.5 standard deviation or more) and levels of repair that are less than complete. This may include, for example and without limitation, effecting an increase in the total abundance of Bacteroides species measured in a fecal sample obtained from a subject after the subject has consumed at least 3 grams or at least 6 grams of total dietary fiber per day in the form of a composition of Section I or Section III, or a food composition of Section I, II, or III. Promoting a healthy gut microbiota in a subject also includes preventing the development of an unhealthy gut microbiota in a subject. In preferred embodiments, the microbiota of a subject is changed with regards to relative abundances of microbial community members and/or expression of proteins encoded by PULs or members of CAZymes families, for instance as detailed in the Examples.

To “improve a subject's health” means to change one or more aspects of a subject's health in a manner towards healthy subjects with similar environmental exposures, such as geography, diet, and age. The improved aspect of the subject's health may be an improvement in weight management, chronic inflammation, cardiovascular health, satiety, and/or glucose metabolism. Non-limiting examples of measurable improvements in weight management may be a reduction in total body weight, a reduction in BMI, a reduction in weight gain, a reduction in fat mass gain, an increase in lean mass, a decrease in waist circumference, a decrease in waist to hip ratio, an increase in adiponectin levels, an increase in leptin levels, a decrease in resistin levels, or any combination thereof. Non-limiting examples of measurable improvements in chronic inflammation include a decrease in one or more plasma protein selected from CCL3, CRP, SPP1, F2, F3, VEGFA, PDGFRB, EFNA5, EPHA1, EPHA2, IL-6, IL-8, IL-1b, IL-1R1, IL-12, IL-17, IL-18, TNF-α, NF-kB, IFN-γ, and ceramides. Non-limiting examples of measurable improvements in cardiovascular health include a decrease in one or more plasma protein selected from C3, C1R, C4A/C4B, F3, SERPINE1, MASP1, PDGFRA, ICAM-1, VCAM-1, MCP-1, PAI-1, P-selectin, thromboxane-A2, F2a-isoprostanes, TBARS, MDA, as well as changes in LDL-cholesterol, HDL-cholesterol, total cholesterol, oxidized LDL, triglycerides, platelet aggregation and blood clotting. Non-limiting examples of measurable improvements in glucose metabolism include changes in fasting glucose, postprandial glucose, fasting insulin, postrprandial glucose, HOMAIR, HbA1c, glycated albumin, fructosamine, glucagon, QIUCKI, ISI, GIP, and GLP-1. Non-limiting examples of measurable improvements in satiety include improvements in AGRP, appetite VAS scores, food intake, GLP-1, PYY, GIP, ghrelin, cholecystokinin and leptin. In some examples, the improved aspect of the subject's health may be a reduction in total body weight, a reduction in BMI, a reduction in weight gain, a reduction in fat mass gain, an increase in fecal levels of succinate, a decrease in serum cholesterol, an increase in insulin sensitivity, a decrease in plasma markers of inflammation, an improvement in the relative abundances of health discriminatory plasma proteins, and/or an improvement in biomarkers/mediators of gut barrier function.

In a specific embodiment, the present disclosure provides a method of decreasing weight gain of a subject on a Western diet, the method comprising administering to the subject a composition comprising at least 15 wt % of one or more pea fiber preparation or a glycan equivalent thereof; and at least one additional fiber preparation chosen from (i) between 0 wt % and 28 wt % (inclusive) of one or more high molecular weight inulin preparation or a glycan equivalent thereof; (ii) between 0 wt % and 10 wt % (inclusive) of one or more citrus pectin preparation or a glycan equivalent thereof; (iii) between 0 wt % and 25 wt % (inclusive) of one or more citrus fiber preparation or a glycan equivalent thereof; or (iv) between 0 wt % and 45 wt % (inclusive) of one or more barley fiber preparation or a glycan equivalent thereof, wherein the administration is at least once a day, in conjunction with the Western diet, for at least 5 days, when weight gain is measured against a population of similar subjects on the same diet without administration of said composition.

In another specific embodiment, the present disclosure provides a method of decreasing the abundance of one or more plasma proteins involved in inflammation in a subject, the method comprising administering to the subject, at least once daily for at least five days, a composition comprising at least 15 wt % of one or more pea fiber preparation or a glycan equivalent thereof; and at least one additional fiber preparation chosen from (i) between 0 wt % and 28 wt % (inclusive) of one or more high molecular weight inulin preparation or a glycan equivalent thereof; (ii) between 0 wt % and 10 wt % (inclusive) of one or more citrus pectin preparation or a glycan equivalent thereof; (iii) between 0 wt % and 25 wt % (inclusive) of one or more citrus fiber preparation or a glycan equivalent thereof; or (iv) between 0 wt % and 45 wt % (inclusive) of one or more barley fiber preparation or a glycan equivalent thereof, wherein the one or more proteins are selected from the group consisting of CCL3, CRP, SPP1, F2, F3, VEGFA, PDGFRB, EFNA5, EPHA1, EPHA2, and IL1R1.

In another specific embodiment, the present disclosure provides a method of treating inflammation in a subject, the method comprising decreasing the abundance of one or more plasma proteins involved in inflammation by administering to the subject, at least once daily for at least five days, a composition comprising at least 15 wt % of one or more pea fiber preparation or a glycan equivalent thereof; and at least one additional fiber preparation chosen from (i) between 0 wt % and 28 wt % (inclusive) of one or more high molecular weight inulin preparation or a glycan equivalent thereof; (ii) between 0 wt % and 10 wt % (inclusive) of one or more citrus pectin preparation or a glycan equivalent thereof; (iii) between 0 wt % and 25 wt % (inclusive) of one or more citrus fiber preparation or a glycan equivalent thereof; or (iv) between 0 wt % and 45 wt % (inclusive) of one or more barley fiber preparation or a glycan equivalent thereof, wherein the one or more proteins are selected from the group consisting of CCL3, CRP, SPP1, F2, F3, VEGFA, PDGFRB, EFNA5, EPHA1, EPHA2, and IL1R1.

In another specific embodiment, the present disclosure provides a method of increasing the representation of one or more CAZyme families in gut microbiome, wherein the one or more CAZyme families are selected from the group consisting of GH43_33, GH116, GH147, GH108, and GH99 activities, the method comprising administering to the subject, at least once daily for at least five days, a composition comprising at least 15 wt % of one or more pea fiber preparation or a glycan equivalent thereof; and at least one additional fiber preparation chosen from (i) between 0 wt % and 28 wt % (inclusive) of one or more high molecular weight inulin preparation or a glycan equivalent thereof; (ii) between 0 wt % and 10 wt % (inclusive) of one or more citrus pectin preparation or a glycan equivalent thereof; (iii) between 0 wt % and 25 wt % (inclusive) of one or more citrus fiber preparation or a glycan equivalent thereof; or (iv) between 0 wt % and 45 wt % (inclusive) of one or more barley fiber preparation or a glycan equivalent thereof.

In another specific embodiment, the present disclosure provides a method of decreasing the abundance of one or more plasma proteins involved in platelet activation and blood coagulation in a subject, the method comprising administering to the subject, at least once daily for at least five days, a composition comprising at least 15 wt % of one or more pea fiber preparation or a glycan equivalent thereof; and at least one additional fiber preparation chosen from (i) between 0 wt % and 28 wt % (inclusive) of one or more high molecular weight inulin preparation or a glycan equivalent thereof; (ii) between 0 wt % and 10 wt % (inclusive) of one or more citrus pectin preparation or a glycan equivalent thereof; (iii) between 0 wt % and 25 wt % (inclusive) of one or more citrus fiber preparation or a glycan equivalent thereof; or (iv) between 0 wt % and 45 wt % (inclusive) of one or more barley fiber preparation or a glycan equivalent thereof, wherein the one or more plasma proteins are selected from the group consisting of C3, C1R, C4A/C4B, F3, SERPINE1, MASP1, and PDGFRA.

In another specific embodiment, the present disclosure provides a method of decreasing the abundance of appetite-stimulating agouti-related protein (AGRP) in a subject, the method comprising administering to the subject, at least once daily for at least five days, a composition comprising at least 15 wt % of one or more pea fiber preparation or a glycan equivalent thereof; and at least one additional fiber preparation chosen from (i) between 0 wt % and 28 wt % (inclusive) of one or more high molecular weight inulin preparation or a glycan equivalent thereof; (ii) between 0 wt % and 10 wt % (inclusive) of one or more citrus pectin preparation or a glycan equivalent thereof; (iii) between 0 wt % and 25 wt % (inclusive) of one or more citrus fiber preparation or a glycan equivalent thereof; or (iv) between 0 wt % and 45 wt % (inclusive) of one or more barley fiber preparation or a glycan equivalent thereof.

In another specific embodiment, the present disclosure provides a method of decreasing the abundance of one or more plasma proteins associated with inflammation and cardiovascular disease, wherein the proteins are selected from the group consisting of CCL3 and CRP, the method comprising administering to a subject, at least once daily for at least five days, a composition comprising at least 15 wt % of one or more pea fiber preparation or a glycan equivalent thereof; and at least one additional fiber preparation chosen from (i) between 0 wt % and 28 wt % (inclusive) of one or more high molecular weight inulin preparation or a glycan equivalent thereof; (ii) between 0 wt % and 10 wt % (inclusive) of one or more citrus pectin preparation or a glycan equivalent thereof; (iii) between 0 wt % and 25 wt % (inclusive) of one or more citrus fiber preparation or a glycan equivalent thereof; or (iv) between 0 wt % and 45 wt % (inclusive) of one or more barley fiber preparation or a glycan equivalent thereof.

In each of the above embodiments, the composition may comprise (i) about 25 wt % to about 40 wt % of one or more pea fiber preparation or a glycan equivalent thereof, about 5 wt % to about 15 wt % of one or more citrus fiber preparation or a glycan equivalent thereof, about 30 wt % to about 40 wt % of a high molecular weight inulin preparation or glycan equivalent thereof, about 10 wt % to about 30 wt % of a barley fiber preparation or glycan equivalent thereof; or (ii) about 30 wt % to about 40 wt % of one or more pea fiber preparation or a glycan equivalent thereof, about 10 wt % to about 20 wt % of one or more citrus fiber preparation or a glycan equivalent thereof, about 30 wt % to about 40 wt % of a high molecular weight inulin preparation or glycan equivalent thereof, about 15 wt % to about 25 wt % of a barley fiber preparation or glycan equivalent thereof; or (iii) about 55 wt % to about 65 wt % of one or more pea fiber preparation or a glycan equivalent thereof and about 30 wt % to about 40 wt % of a high molecular weight inulin preparation or glycan equivalent thereof; or (iv) about 60 wt % to about 70 wt % of one or more pea fiber preparation or a glycan equivalent thereof and about 30 wt % to about 40 wt % of a high molecular weight inulin preparation or glycan equivalent thereof; or (v) about 60 wt % to about 65 wt % of one or more pea fiber preparation or a glycan equivalent thereof and about 35 wt % to about 40 wt % of a high molecular weight inulin preparation or glycan equivalent thereof.

In each of the above embodiments, the pea fiber preparation may have a composition substantially similar to the pea fiber preparation of Table A or Table G, and/or a monosaccharide content substantially similar to the pea fiber preparation of Table B or Table G, and optionally glycosyl linkages substantially similar to the pea fiber preparation of Table C1 or Table C2; the high molecular weight inulin preparation has a composition substantially similar to the high molecular weight inulin preparation of Table A or Table G; the barley fiber preparation has a composition substantially similar to the barley fiber preparation of Table A or Table G, and/or a monosaccharide content substantially similar to the barley fiber preparation of Table B or Table G, and optionally glycosyl linkages substantially similar to the barley fiber preparation of Table E; the citrus fiber preparation has a composition substantially similar to the citrus fiber preparation of Table A or Table G, and/or a monosaccharide content substantially similar to the citrus fiber preparation of Table B or Table G, and optionally glycosyl linkages substantially similar to the citrus fiber preparation of Table F1 or Table F2.

In each of the above embodiments, the composition may be administered as part of a food composition. Alternatively, each of the fiber preparations comprising the composition may be individual ingredients in a food composition and the food composition administered to a subject. The duration of administration may vary depending upon a variety of factors, including the severity of disrepair and/or the health of the subject. Typically, the duration of administration may be for at least one week, at least two weeks, at least three weeks, or at least four weeks. In some examples, a composition may be administered for about 1 month, about 2 months, about 3 months, about 4 months or more. In some examples, a composition or food composition may be administered for about 6 months, about 12 months, or more. In some examples, a composition or food composition may be administered for about 1 month to about 6 months. In some examples, a composition or food composition may be administered for about 6 months to about 12 months.

In some of the above embodiments, a subject is a healthy subject (e.g., a healthy BMI, adequate dietary fiber intake, no chronic or acute disease, etc.) looking to promote a healthy gut microbiota.

In some of the above embodiments, a subject has a diet that is high in saturated fats and/or low in fruits and vegetables, a total dietary fiber intake less than 30 grams a day, a total dietary fiber intake less than 25 grams a day, a total dietary fiber intake less than 20 grams a day, a total dietary fiber intake less than 15 grams a day, a total dietary fiber intake less than 10 grams a day, a BMI of 25 or greater, or any combination thereof.

In some of the above embodiments, a subject may have insulin insensitivity, insulin resistance, type I diabetes mellitus, type II diabetes mellitus, systemic inflammation, a chronic inflammatory disease, heart disease, cardiovascular disease, high cholesterol, high blood pressure, or any combination thereof. In some embodiments, a subject may have an increased risk of developing insulin insensitivity, insulin resistance, type I diabetes mellitus, type II diabetes mellitus, systemic inflammation, a chronic inflammatory disease, heart disease, cardiovascular disease, high cholesterol, high blood pressure, or any combination thereof, whether due to family history or lifestyle.

In some of the above embodiments, a subject is prone to having a gut microbiota in disrepair. Subjects prone to have a gut microbiota in disrepair may or may not have a measurable change in a measure of gut microbiota health as compared to reference healthy subjects, and confirmation of the health status of the subject's gut microbiota is not needed. Subjects prone to have a gut microbiota in disrepair include but are not limited to subjects that have a diet that is high in saturated fats and/or low in fruits and vegetables, a total dietary fiber intake less than 30 grams a day, a total dietary fiber intake less than 25 grams a day, a total dietary fiber intake less than 20 grams a day, a total dietary fiber intake less than 15 grams a day, a total dietary fiber intake less than 10 grams a day, a BMI of 25 or greater, insulin insensitivity, insulin resistance, type I diabetes mellitus, type II diabetes mellitus, systemic inflammation or a chronic inflammatory disease, heart disease, cardiovascular disease, high cholesterol, high blood pressure, or any combination thereof.

In some of the above embodiments, a subject has gut microbiota in disrepair. In further embodiments, the subject has a total dietary fiber intake less than 30 grams a day, a total dietary fiber intake less than 25 grams a day, a total dietary fiber intake less than 20 grams a day, a total dietary fiber intake less than 15 grams a day, a total dietary fiber intake less than 10 grams a day, a BMI of 25 or greater, insulin insensitivity, insulin resistance, type I diabetes mellitus, type II diabetes mellitus, systemic inflammation or a chronic inflammatory disease, heart disease, high cholesterol, high blood pressure, or any combination thereof.

In some of the above embodiments, the subject is overweight or obese (e.g., as defined by a BMI outside the normal range for the subject's age, sex, and/or ethnicity). In some of the above embodiments, the subject typically consumes a diet low in total dietary fiber (e.g., less than about 25 g per day). In some of the above embodiments, the subject typically consumes a Western diet. An exemplary Western diet is the HiSF/LoFV diet detailed in the examples that is suitable for animals), and human equivalents thereof.

### Numbered Embodiments

1. A composition comprising a plurality of fiber preparations, each fiber preparation independently selected from the group consisting of a barley fiber preparation, a citrus fiber preparation, a citrus pectin formulation, a high molecular weight inulin preparation, a pea fiber preparation, and a sugar beet fiber preparation, wherein the plurality of fiber preparations is at least 95 wt % of the composition.

2. The composition of embodiment 1, wherein the composition comprises one or more citrus pectin preparation in an amount that does not exceed 10 wt %.

3. The composition of embodiment 1, wherein the composition comprises one or more citrus fiber preparation in an amount that does not exceed 25 wt %.

4. The composition of embodiment 1, wherein the composition comprises at least 15 wt % of one or more pea fiber preparation.

5. The composition of embodiment 1, wherein the composition comprises at least 28 wt % of one or more high molecular weight inulin preparation.

6. The composition of embodiment 1, wherein the composition comprises one or more barley fiber preparation in an amount that does not exceed 45 wt %.

7. The composition of embodiment 1, wherein the composition comprises at least 15 wt % of one or more sugar beet fiber preparation.

8. The composition of embodiment 1, wherein the composition comprises (a) at least 15 wt % of one or more pea fiber preparation and at least 28 wt % of one or more high molecular weight inulin preparation; (b) the total amount of citrus pectin preparations does not exceed 10 wt %, (c) the total amount of citrus fiber preparations does not exceed 25 wt %, and (d) the total amount of barley fiber preparations does not exceed 45 wt %.

9. The composition of embodiment 1, wherein the composition comprises at least 15 wt % of one or more sugar beet fiber preparation and at least 28 wt % of one or more high molecular weight inulin preparation; the total amount of citrus pectin preparations does not exceed 10 wt %, the total amount of citrus fiber preparations does not exceed 25 wt %, and the total amount of barley fiber preparations does not exceed 45 wt %.

10. A composition comprising at least 15 wt % of one or more pea fiber preparation or a glycan equivalent thereof; and at least one additional fiber preparation chosen from (i) at least 28 wt % of one or more high molecular weight inulin preparation or a glycan equivalent thereof, (ii) 10 wt % or less of one or more citrus pectin preparation or a glycan equivalent thereof, (iii) 25 wt % or less of one or more citrus fiber preparation or a glycan equivalent thereof, or (iv) 45 wt % or less of one or more barley fiber preparation or a glycan equivalent thereof.

11. The composition of embodiment 10, wherein there is at least 28 wt % of one or more pea fiber preparation, or a glycan equivalent thereof.

12. The composition of embodiment 10, wherein there is at least 30 wt % of one or more pea fiber preparation, or a glycan equivalent thereof; and there is at least 30 wt % of one or more high molecular weight inulin preparation, or a glycan equivalent thereof.

13. The composition of any of embodiments 10-12, wherein there is less than 1 wt % of one or more citrus pectin preparation, or a glycan equivalent thereof.

14. The composition of any of embodiments 10-12, wherein there is no citrus pectin preparation, or a glycan equivalent thereof.

15. The composition of any of embodiments 10-14, wherein there is 15 wt % or less of one or more citrus fiber preparation, or a glycan equivalent thereof.

16. The composition of embodiment 15, wherein there is 12 wt % or less of one or more citrus fiber preparation, or a glycan equivalent thereof.

17. The composition of any one of embodiments 10-16, wherein there is 25 wt % or less of one or more barley fiber preparation, or glycan equivalent thereof.

18. The composition of embodiment 17, wherein there is 25 wt % or less of one or more barley fiber preparation, or glycan equivalent thereof.

19. A composition comprising about 35 wt % of one or more pea fiber preparation or a glycan equivalent thereof, about 10 wt % of one or more citrus fiber preparation or a glycan equivalent thereof, about 35 wt % of one or more high molecular weight inulin preparation or a glycan equivalent thereof, and about 20 wt % of one or more barley fiber preparation or a glycan equivalent thereof; and wherein the pea fiber preparation(s), citrus fiber preparation(s), high molecular weight inulin preparation(s), and barley fiber preparation(s) are at least 95 wt % of the composition.

20. A composition comprising about 30-40 wt % of one or more pea fiber preparation or a glycan equivalent thereof, about 9-11 wt % of one or more citrus fiber preparation or a glycan equivalent thereof, about 30-40 wt % of one or more high molecular weight inulin or a glycan equivalent thereof, and about 18-22 wt % of one or more barley fiber preparation or a glycan equivalent thereof; and wherein the pea fiber preparation(s), citrus fiber preparation(s), high molecular weight inulin preparation(s), and barley fiber preparation(s) are at least 95 wt % of the composition.

21. A composition comprising about 30-35 wt % of one or more pea fiber preparation or a glycan equivalent thereof, about 9-11 wt % of one or more citrus fiber preparation or a glycan equivalent thereof, about 35-40 wt % of one or more high molecular weight inulin preparation or a glycan equivalent thereof, and about 18-22 wt % of one or more barley bran preparation or a glycan equivalent thereof; and wherein the pea fiber preparation(s), citrus fiber preparation(s), high molecular weight inulin preparation(s), and barley fiber preparation(s) are at least 95 wt % of the composition.

22. A composition comprising about 33 wt % of one or more pea fiber preparation or a glycan equivalent thereof, about 11 wt % of one or more citrus fiber preparation or a glycan equivalent thereof, about 36 wt % of one or more high molecular weight inulin preparation or a glycan equivalent thereof, and about 20 wt % of one or more barley fiber preparation or a glycan equivalent thereof; and wherein the pea fiber preparation(s), citrus fiber preparation(s), high molecular weight inulin preparation(s), and barley fiber preparation(s) are at least 95 wt % of the composition.

23. A composition comprising about 65 wt % pea fiber or a glycan equivalent thereof, and about 35 wt % high molecular weight inulin or a glycan equivalent thereof; and wherein the pea fiber preparation(s) and high molecular weight inulin preparation(s) are at least 95 wt % of the composition.

24. A food comprising a composition of any one of the preceding claims.

25. A baked, pressed or extruded food comprising a composition of any one of embodiments 1 to 23.

26. The food of embodiment 24 or 25, wherein the amount of the composition is about 40 wt % to about 50 wt % of the food.

27. The food of embodiment 26, wherein the amount of the composition is about 45 wt % to about 50 wt % of the food.

28. The food of embodiment 24, 25, 26, or 27, wherein the composition provides about 90% or more of the total dietary fibers in the food.

29. The food of embodiment 28, wherein the dietary fiber blend provides about 95% or more of the total dietary fibers in the composition.

30. The food of embodiment 29, wherein the dietary fiber blend provides about 98% or more of the total dietary fibers in the composition.

31. A pressed, extruded or baked food, the food comprising about 40 wt % to about 95 wt % of a composition of fiber preparations, the composition of fiber preparations comprising (a) about 25 wt % to about 40 wt % of one or more pea fiber preparation, or a glycan equivalent thereof; about 5 wt % to about 15 wt % of one or more citrus fiber preparation, or a glycan equivalent thereof; about 30 wt % to about 40 wt % of one or more high molecular weight inulin preparation, or a glycan equivalent thereof; and about 10 wt % to about 30 wt % of one or more barley fiber preparation, or a glycan equivalent thereof; or (b) about 55 wt % to about 65 wt % of one or more pea fiber preparation, or a glycan equivalent thereof; and about 30 wt % to about 40 wt % of one or more high molecular weight inulin preparation, or a glycan equivalent thereof; wherein a 30 g serving of the food has at least 6 g of total dietary fiber; and wherein the food effects an increase in the fiber degrading capacity of a subject's gut microbiota and/or an improvement in the a subject's health, when the subject has consumed the food at least once a day for at least 7 days.

32. The food of embodiment 31, wherein the composition of fiber preparations provides about 90% or more of the total dietary fiber in the food.

33. The food of embodiment 31, wherein the composition of fiber preparations provides about 95% or more of the total dietary fiber in the composition.

34. The food of embodiment 31, wherein the composition of fiber preparations provides about 98% or more of the total dietary fiber in the food.

35. The food of any one of embodiments 31 to 34, wherein the composition of fiber preparations comprises (i) about 30 wt % to about 35 wt % of one or more pea fiber preparation, or a glycan equivalent thereof, (ii) about 9 wt % to about 11 wt % of one or more citrus fiber preparation, or a glycan equivalent thereof, (iii) about 35 wt % to about 40 wt % of one or more high molecular weight inulin preparation, or a glycan equivalent thereof, and about 18 wt % to about 22 wt % of one or more barley fiber preparation, or a glycan equivalent thereof; and wherein the pea fiber preparation(s), citrus fiber preparation(s), high molecular weight inulin preparation(s), and barley fiber preparation(s) are at least 95 wt % of the composition.

36. The food of any one of embodiments 31 to 34, wherein the composition of fiber preparations comprises about 33 wt % of one or more pea fiber preparation, or a glycan equivalent thereof, about 11 wt % of one or more orange fiber preparation, or a glycan equivalent thereof, about 36 wt % of one or more high molecular weight inulin preparation, or a glycan equivalent thereof, and about 20 wt % of one or more barley fiber preparation, or a glycan equivalent thereof; and wherein the pea fiber preparation(s), citrus fiber preparation(s), high molecular weight inulin preparation(s), and barley fiber preparation(s) are at least 95 wt % of the composition.

37. The food of any one of embodiments 31 to 34, wherein the composition of fiber preparations comprises about 30 wt % to about 35 wt % of one or more pea fiber preparation, about 9 wt % to about 11 wt % of one or more citrus fiber preparation, about 35 wt % to about 40 wt % of one or more high molecular weight inulin preparation, and about 18-22 wt % of one or more barley fiber preparation; and wherein the pea fiber preparation(s), citrus fiber preparation(s), high molecular weight inulin preparation(s), and barley fiber preparation(s) are at least 95 wt % of the composition.

38. The food of any one of embodiments 31 to 34, wherein the composition of fiber preparations comprises about 33 wt % of one or more pea fiber preparation, about 11 wt % of one or more citrus fiber preparation, about 36 wt % of one or more high molecular weight inulin preparation, and about 20 wt % of one or more barley fiber preparation; and wherein the pea fiber preparation(s), citrus fiber preparation(s), high molecular weight inulin preparation(s), and barley fiber preparation(s) are at least 95 wt % of the composition.

39. The food of any one of embodiments 31 to 34, wherein the composition of fiber preparations comprises about 60 wt % to about 65 wt % of one or more pea fiber preparation, or a glycan equivalent thereof; and about 30 wt % to about 35 wt % of one or more high molecular weight inulin preparation, or a glycan equivalent thereof; and wherein the pea fiber preparation(s), citrus fiber preparation(s), high molecular weight inulin preparation(s), and barley fiber preparation(s) are at least 95 wt % of the composition.

40. The food of any one of embodiments 31 to 34, wherein the composition of fiber preparations comprises about 65 wt % of one or more pea fiber preparation, or a glycan equivalent thereof; and about 35 wt % of one or more high molecular weight inulin preparation, or a glycan equivalent thereof.

41. The food of any one of embodiments 31 to 34, wherein the composition of fiber preparations comprises about 60 wt % to about 65 wt % of one or more pea fiber preparation; and about 30 wt % to about 35 wt % of one or more high molecular weight inulin preparation; and wherein the pea fiber preparation(s), citrus fiber preparation(s), high molecular weight inulin preparation(s), and barley fiber preparation(s) are at least 95 wt % of the composition.

42. The food of any one of embodiments 31 to 34, wherein the composition of fiber preparations comprises about 65 wt % of one or more pea fiber preparation, and about 35 wt % of one or more high molecular weight inulin preparation; and wherein the pea fiber preparation(s), citrus fiber preparation(s), high molecular weight inulin preparation(s), and barley fiber preparation(s) are at least 95 wt % of the composition.

43. The food of any one of embodiments 24 to 42, wherein the food further comprises flour(s), meal(s), oil(s), fat(s), inclusions, sweetener(s), starch(es), salt(s), emulsifier(s), leavening agent(s), preservative(s) or combinations thereof.

44. The food of embodiment 43, wherein the food comprises one or more flour and/or meal in an amount that is about 10 wt % to about 60 wt % of the food.

45. The food of embodiment 44, wherein the one or more flour is chosen from wheat four, rice flour, corn flour, or any combination thereof.

46. The food of any one of embodiments 43 to 45, wherein the food comprises one or more sweetener in an amount that is about 0.005 wt % to about 40 wt % of the food.

47. The food of embodiment 46, wherein the one or more sweetener is sugar.

48. The food of any one of embodiments 43 to 47, wherein the food comprises one or more salt in an amount that is about 0.5 wt % to about 5 wt % of the food.

49. The food of embodiment 48, wherein the one or more salt is sodium chloride.

50. The food of any one of embodiments 43 to 49, wherein the food comprises one or more emulsifier in an amount that is about 0.1 wt % to about 2 wt % of the food.

51. The food of embodiment 50, wherein the one or more emulsifier is chosen from glycerol monostearate, lecithin, polysorbate, or other mono or diglycerides.

52. The food of any one of embodiment 43 to 49, wherein the food comprises one or more leavening agent in an amount that is about 0.1 wt % to about 5 wt % of the food.

53. The food of embodiment 52, wherein the one or more leavening agent is chosen from sodium bicarbonate, monocalcium phosphate, or calcium carbonate, ammonium bicarbonate, mono calcium phosphate monohydrate, sodium acid pyrophosphate, sodium aluminum phosphate, organic acids, and yeast.

54. The food of any one of embodiments 43 to 54, wherein the food further comprises a color additive, a flavor, a flavor enhancer, a stabilizer, a humectant, a firming agent, an enzyme, a probiotic, a spice, a binder, fruit, vegetables, grains, vitamins, minerals or combinations thereof.

55. The food of any one of embodiments 43 to 54, wherein food is a baked food that has about 6 g to about 10 g of fiber in a 30 g serving.

56. The baked food of embodiment 55, wherein the baked food has about 6 g of fiber in a 30 g serving.

57. The baked food of embodiment 55, wherein the baked food has about 10 g of fiber in a 30 g serving.

58. The baked food of any one embodiments 55 to 57, wherein the baked food is a cracker, a cookie, a cake, a bar, a bread, or a muffin.

59. The food of any one of embodiments 43 to 54, wherein food is an extruded food that has about 6 g to about 10 g of fiber in a 30 g serving.

60. The extruded food of embodiment 59, wherein the extruded food has about 6 g of fiber in a 30 g serving.

61. The extruded food of embodiment 59, wherein the extruded food has about 10 g of fiber in a 30 g serving.

62. The extruded food of any one of embodiments 59 to 61, wherein the extruded food is an extruded pillow or any other extruded shape.

63. A pea fiber preparation for use in any one of embodiments 1 to 62, wherein about 55 wt % to about 65 wt % of the total dietary fiber in the pea fiber preparation is insoluble dietary fiber, and/or about 60 wt % to about 70 wt % of the total dietary fiber in the pea fiber preparation is high molecular dietary fiber.

64. The pea fiber preparation of embodiment 63, wherein the pea fiber preparation has a monosaccharide content that is substantially similar to the preparation of Table B.

65. The pea fiber preparation of embodiment 63 or 64, wherein the pea fiber preparation has glycosidic linkages substantially similar to the preparation of Table D, Table 13, Table 14, Table 16, or Table 17.

66. The pea fiber preparation of embodiment 63, 64 or 65, wherein the pea fiber preparation comprises arabinan of formula (I)

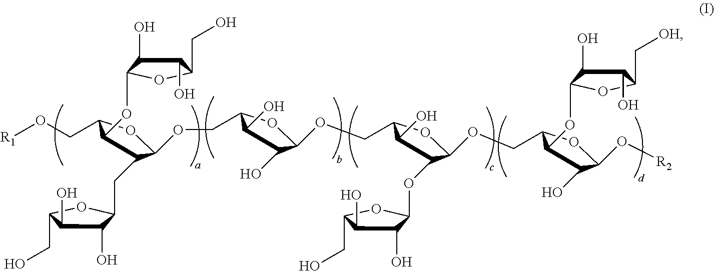

wherein a is about 0.1 to about 0.3, b is about 0.4 to about 0.6, c is about 0.1 to about 0.4, d is about 0.04 to about 0.06; and wherein R₁ and R₂ are each independently selected from H, a glycosyl, a sugar moiety (modified or not), an oligosaccharide (branched or not), or a polysaccharide (branched or not), and a polysaccharide containing galacturonic acid, galactose, and rhamnose.

67. A glycan equivalent of a pea fiber preparation for use in any one of embodiments 1 to 62, wherein the glycan equivalent is a compositional glycan equivalent of a pea fiber preparation of any one of embodiments 63 to 66.

68. A glycan equivalent of a pea fiber preparation for use in any one of embodiments 1 to 62, wherein the glycan equivalent is a functional glycan equivalent of a pea fiber preparation of any one of embodiments 63 to 66.

69. A citrus fiber preparation for use in any one of embodiments 1 to 62, wherein about 30 wt % to about 40 wt % of the total dietary fiber in the citrus fiber preparation is insoluble dietary fiber, and/or about 65 wt % to about 75 wt % of the total dietary fiber in the citrus fiber preparation is high molecular dietary fiber.

70. The citrus fiber preparation of embodiment 69, wherein the citrus fiber preparation has a monosaccharide content that is substantially similar to the preparation of Table B.

71. The citrus fiber preparation of embodiment 69 or 70, wherein the citrus fiber preparation has glycosidic linkages substantially similar to the preparation of Table F.

72. A citrus pectin preparation for use in any one of embodiments 1 to 62, wherein about 1 wt % to about 10 wt % of the total dietary fiber in the citrus pectin preparation is insoluble dietary fiber, and/or about 85 wt % to about 95 wt % of the total dietary fiber in the citrus pectin preparation is high molecular dietary fiber.

73. The citrus pectin preparation of embodiment 72, wherein the citrus pectin preparation has a monosaccharide content that is substantially similar to the citrus pectin preparation of Table B.

74. The citrus pectin preparation of embodiment 72 or 73, wherein the citrus pectin preparation has glycosidic linkages substantially similar to the preparation exemplified in Table D.

75. A barley fiber preparation for use in any one of embodiments 1 to 62, wherein about 5 wt % to about 15 wt % of the total dietary fiber in the barley fiber preparation is insoluble dietary fiber, and/or about 40 wt % to about 45 wt % of the total dietary fiber in the barley fiber preparation is high molecular dietary fiber.

76. The barley fiber preparation of embodiment 75, wherein the barley fiber preparation has a monosaccharide content that is substantially similar to the preparation of Table B.

77. The barley fiber preparation of embodiment 75 or 76, wherein the barley fiber preparation has glycosidic linkages substantially similar to the preparation exemplified in Table E.

78. A high molecular weight inulin preparation for use in any one of embodiments 1 to 62, wherein the total dietary fiber in the high molecular weight inulin preparation is about 85 wt % to about 99 wt %.

79. The high molecular weight inulin preparation of embodiment 78, wherein the high molecular weight inulin preparation has a degree of polymerization greater than 5.

80. A sugar beet fiber preparation for use in any one of embodiments 1 to 62, wherein about 55 wt % to about 65 wt % of the total dietary fiber in the sugar beet fiber preparation is insoluble dietary fiber, and/or about 75 wt % to about 85 wt % of the total dietary fiber in the sugar beet fiber preparation is high molecular dietary fiber.

81. A composition of any one of embodiments 1 to 23, wherein the composition (i) effects an increase in the total abundance of Bacteroides species measured in a fecal sample obtained from a subject after the subject has consumed the composition at least once a day for at least 7 days, as compared to the total abundance of Bacteroides species measured in a fecal sample obtained from the subject prior to consumption of the composition, (ii) effects an increase in the relative abundance of Bacteroides species measured in a fecal sample obtained from a subject after the subject has consumed the composition at least once a day for at least 7 days, as compared to the relative abundance of Bacteroides species measured in a fecal sample obtained from the subject prior to consumption of the composition, or (iii) effects a health improvement in a subject after the subject has consumed the composition at least once a day for at least 7 days.

82. The composition of embodiment 81, wherein the composition effects a health improvement in a subject after the subject has consumed the composition at least once a day for 7 days, the health improvement being selected from a reduction in total body weight, a reduction in BMI, a reduction in fat mass gain, an increase in fecal levels of succinate, a decrease in serum cholesterol, an increase in insulin sensitivity, a decrease in plasma markers of inflammation, an improvement in the relative abundances of health discriminatory plasma proteins, and/or an improvement in biomarkers/mediators of gut barrier function.

83. The food of embodiment 31, wherein the food effects an increase in the total abundance of Bacteroides species measured in a fecal sample obtained from a subject after the subject has consumed the food at least once a day for at least 7 days.

84. The food of embodiment 31, wherein the food effects an increase in the relative abundance of Bacteroides species measured in a fecal sample obtained from a subject after the subject has consumed the food at least once a day for at least 7 days.

85. The food of embodiment 31, wherein the food effects an increase in the total abundance of Bacteroides species measured in a fecal sample obtained from a subject after the subject has consumed the food at least once a day for at least 14 days.

86. The food of embodiment 31, wherein the food effects an increase in the relative abundance of Bacteroides species measured in a fecal sample obtained from a subject after the subject has consumed the food at least once a day for at least 14 days.

### EXAMPLES

The following examples illustrate various iterations of the invention and in some instances demonstrate preferred embodiments of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

The references listed below are cited in the Examples that follow.
Adey, A., Morrison, H. G., Asan, Xun, X., Kitzman, J. O., Turner, E. H., Stackhouse, B., MacKenzie, A. P., Caruccio, N.C., Zhang, X., et al. (2010). Rapid, low-input, low-bias construction of shotgun fragment libraries by high-density in vitro transposition. Genome Biol 11, R119.
Atmodjo, M. A., Hao, Z., and Mohnen, D. (2013). Evolving views of pectin biosynthesis. Annu Rev Plant Biol 64, 747-779.
Bagwe, R. P., Hilliard, L. R., and Tan, W. H. (2006) Surface modification of silica nanoparticles to reduce aggregation and nonspecific binding. Langmuir 22, 4357-4362.
Bokulich, N. A., Subramanian, S., Faith, J. J., Gevers, D., Gordon, J. I., Knight, R., Mills, D. A., and Caporaso, J. G. (2013). Quality-filtering vastly improves diversity estimates from IIlumina amplicon sequencing. Nature Methods 10, 57-59.
Buffetto, F., Cornuault, V., Rydahl, M. G., Ropartz, D., Alvarado, C., Echasserieau, V., Le Gall, S., Bouchet, B., Tranquet, O., Verhertbruggen, Y., et al. (2015). The Deconstruction of Pectic Rhamnogalacturonan I Unmasks the Occurrence of a Novel Arabinogalactan Oligosaccharide Epitope. Plant Cell Physiol 56, 2181-2196.
Clarkson, S. M., Giannone, R. J., Kridelbaugh, D. M., Elkins, J. G., Guss, A. M., and Michener, J. K. (2017). Construction and Optimization of a Heterologous Pathway for Protocatechuate Catabolism in Escherichia coli Enables Bioconversion of Model Aromatic Compounds. Applied and Environmental Microbiol 83, e01313-01317.
Desai, M. S., Seekatz, A. M., Koropatkin, N. M., Kamada, N., Hickey, C. A., Wolter, M., Pudlo, N. A., Kitamoto, S., Terrapon, N., Muller, A., et al. (2016). A Dietary Fiber-Deprived Gut Microbiota Degrades the Colonic Mucus Barrier and Enhances Pathogen Susceptibility. Cell 167, 1339-1353 e1321.
Doares, S. H., Albersheim, P., and Darvill, A. G. (1991) An Improved Method for the Preparation of Standards for Glycosyl-Linkage Analysis of Complex Carbohydrates. Carbohydr Res 210, 311-317.
Doco, T., O'Neill, M. A., and Pellerin, P. (2001) Determination of the neutral and acidic glycosyl-residue compositions of plant polysaccharides by GC-EI-MS analysis of the trimethylsilyl methyl glycoside derivatives. Carbohydr Polym 46, 249-259
Edgar, R. C. (2010). Search and clustering orders of magnitude faster than BLAST. Bioinformatics 26, 2460-2461.
Englyst, H. N., and Cummings, J. H. (1988). Improved Method for Measurement of Dietary Fiber as Non-Starch Polysaccharides in Plant Foods. Journal of the Association of Official Analytical Chemists 71, 808-814.
Faith, J. J., Ahern, P. P., Ridaura, V. K., Cheng, J., and Gordon, J. I. (2014). Identifying gut microbe-host phenotype relationships using combinatorial communities in gnotobiotic mice. Sci Transl Med 6, 220ra211.
Faith, J. J., Guruge, J. L., Charbonneau, M., Subramanian, S., Seedorf, H., Goodman, A. L., Clemente, J. C., Knight, R., Heath, A. C., Leibel, R. L., et al. (2013). The long-term stability of the human gut microbiota. Science 341, 1237439.
Filisetti-Cozzi, T. M., and Carpita, N.C. (1991). Measurement of uronic acids without interference from neutral sugars. Anal Biochem 197, 157-162.
Fragiadakis, G. K., Smits, S. A., Sonnenburg, E. D., Van Treuren, W., Reid, G., Knight, R., Manjurano, A., Changalucha, J., Dominguez-Bello, M. G., Leach, J., et al. (2018). Links between environment, diet, and the hunter-gatherer microbiome. Gut Microbes, 1-12.
Glabe, C. G., Harty, P. K., and Rosen, S. D. (1983) Preparation and properties of fluorescent polysaccharides. Analytical Biochemistry 130, 287-294.
Glenwright, A. J., Pothula, K. R., Bhamidimarri, S. P., Chorev, D. S., Basle, A., Firbank, S. J., Zheng, H., Robinson, C. V., Winterhalter, M., Kleinekathofer, U., Bolam, D. N., et al. (2017). Structural basis for nutrient acquisition by dominant members of the human gut microbiota. Nature 541, 407-411.
Goodman, A. L., McNulty, N. P., Zhao, Y., Leip, D., Mitra, R. D., Lozupone, C. A., Knight, R., and Gordon, J. I. (2009). Identifying genetic determinants needed to establish a human gut symbiont in its habitat. Cell Host & Microbe 6, 279-289.
Hibberd, M. C., Wu, M., Rodionov, D. A., Li, X., Cheng, J., Griffin, N. W., Barratt, M. J., Giannone, R. J., Hettich, R. L., Osterman, A. L., et al. (2017). The effects of micronutrient deficiencies on bacterial species from the human gut microbiota. Sci Transl Med 9.
Hibbing, M. E., Fuqua, C., Parsek, M. R., and Peterson, S. B. (2010). Bacterial competition: surviving and thriving in the microbial jungle. Nature Reviews Microbiology 8, 15-25.
Koniev, O., and Wagner, A. (2015) Developments and recent advancements in the field of endogenous amino acid selective bond forming reactions for bioconjugation. Chem Soc Rev 44, 5495-5551.
Kotarski, S. F., and Salyers, A. A. (1984). Isolation and characterization of outer membranes of Bacteroides thetaiotaomicron grown on different carbohydrates. Journal of Bacteriology 158, 102-109.
Law, C. W., Chen, Y., Shi, W., and Smyth, G. K. (2014). voom: Precision weights unlock linear model analysis tools for RNA-seq read counts. Genome Biol 15, R29.
Lees, A., Nelson, B. L., and Mond, J. J. (1996) Activation of soluble polysaccharides with 1-cyano-4-dimethylaminopyridinium tetrafluoroborate for use in protein-polysaccharide conjugate vaccines and immunological reagents. Vaccine 14, 190-198.
Levigne, S., Thomas, M., Ralet, M. C., Quemener, B. C., and Thibault, J. F. (2002). Determination of the degrees of methylation and acetylation of pectins using a C18 column and internal standards. Food Hydrocolloids 16, 547-550.
Luis, A. S., Briggs, J., Zhang, X., Farnell, B., Ndeh, D., Labourel, A., Basle, A., Cartmell, A., Terrapon, N., Stott, K., et al. (2018). Dietary pectic glycans are degraded by coordinated enzyme pathways in human colonic Bacteroides. Nature Microbiol 3, 210-219.
Lynch, J. B., and Sonnenburg, J. L. (2012). Prioritization of a plant polysaccharide over a mucus carbohydrate is enforced by a Bacteroides hybrid two-component system. Mol Microbiol 85, 478491.
Ma, Z. Q., Dasari, S., Chambers, M. C., Litton, M. D., Sobecki, S. M., Zimmerman, L. J., Halvey, P. J., Schilling, B., Drake, P. M., Gibson, B. W., et al. (2009). IDPicker 2.0: Improved protein assembly with high discrimination peptide identification filtering. J Proteome Res 8, 3872-3881.
Martens, E. C., Lowe, E. C., Chiang, H., Pudlo, N. A., Wu, M., McNulty, N. P., Abbott, D. W., Henrissat, B., Gilbert, H. J., Bolam, D. N., et al. (2011). Recognition and degradation of plant cell wall polysaccharides by two human gut symbionts. PLoS Biology 9, e1001221.
Masuko, T., Minami, A., Iwasaki, N., Majima, T., Nishimura, S., and Lee, Y. C. (2005) Carbohydrate analysis by a phenol-sulfuric acid method in microplate format. Anal Biochem 339, 69-72.
McDonald, D., Price, M. N., Goodrich, J., Nawrocki, E. P., DeSantis, T. Z., Probst, A., Andersen, G. L., Knight, R., and Hugenholtz, P. (2012). An improved Greengenes taxonomy with explicit ranks for ecological and evolutionary analyses of bacteria and archaea. ISME J 6, 610-618.
McNulty, N. P., Wu, M., Erickson, A. R., Pan, C., Erickson, B. K., Martens, E. C., Pudlo, N. A., Muegge, B. D., Henrissat, B., Hettich, R. L., et al. (2013). Effects of diet on resource utilization by a model human gut microbiota containing Bacteroides cellulosilyticus WH2, a symbiont with an extensive glycobiome. PLoS Biology 11, e1001637.
Menni, C., Jackson, M. A., Pallister, T., Steves, C. J., Spector, T. D., and Valdes, A. M. (2017). Gut microbiome diversity and high-fiber intake are related to lower long-term weight gain. Int J Obes (Lond) 41, 1099-1105.
Pattathil, S., Avci, U., Miller, J. S., and Hahn, M. G. (2012) Immunological approaches to plant cell wall and biomass characterization: Glycome Profiling. Methods Mol Biol 908, 61-72
Pettolino, F. A., Walsh, C., Fincher, G. B., and Bacic, A. (2012). Determining the polysaccharide composition of plant cell walls. Nat Protoc 7, 1590-1607.
Porter, N. T., and Martens, E. C. (2017). The critical roles of polysaccharides in gut microbial ecology and physiology. Annu Rev Microbiol 71, 349-369.
Ridaura, V. K., Faith, J. J., Rey, F. E., Cheng, J., Duncan, A. E., Kau, A. L., Griffin, N. W., Lombard, V., Henrissat, B., Bain, J. R., et al. (2013). Gut microbiota from twins discordant for obesity modulate metabolism in mice. Science 341, 1241214.
Rogowski, A., Briggs, J. A., Mortimer, J. C., Tryfona, T., Terrapon, N., Lowe, E. C., Basle, A., Morland, C., Day, A. M., Zheng, H., et al. (2015). Glycan complexity dictates microbial resource allocation in the large intestine. Nature Commun 6, 7481.
Schwalm, N. D., 3rd, Townsend, G. E., 2nd, and Groisman, E. A. (2016). Multiple Signals Govern Utilization of a Polysaccharide in the Gut Bacterium Bacteroides thetaiotaomicron. MBio 7.
Shafer, D. E., Toll, B., Schuman, R. F., Nelson, B. L., Mond, J. J., and Lees, A. (2000) Activation of soluble polysaccharides with 1-cyano-4-dimethylaminopyridinium tetrafluoroborate (CDAP) for use in protein-polysaccharide conjugate vaccines and immunological reagents. II. Selective crosslinking of proteins to CDAP-activated polysaccharides. Vaccine 18, 1273-1281.
Shepherd, E. S., DeLoache, W. C., Pruss, K. M., Whitaker, W. R., and Sonnenburg, J. L. (2018). An exclusive metabolic niche enables strain engraftment in the gut microbiota. Nature 557, 434-438.
Smyth, G. K. (2004). Linear models and empirical bayes methods for assessing differential expression in microarray experiments. Stat Appl Genet Mol Biol 3, Article3.
Sonnenburg, E. D., Smits, S. A., Tikhonov, M., Higginbottom, S. K., Wingreen, N. S., and Sonnenburg, J. L. (2016). Diet-induced extinctions in the gut microbiota compound over generations. Nature 529, 212-215.
Soto-Cantu, E., Cueto, R., Koch, J., and Russo, P. S. (2012) Synthesis and Rapid Characterization of Amine-Functionalized Silica. Langmuir 28, 5562-5569.
Tabb, D. L., Fernando, C. G., and Chambers, M. C. (2007). MyriMatch: highly accurate tandem mass spectral peptide identification by multivariate hypergeometric analysis. J Proteome Res 6, 654-661.
Tauzin, A. S., Laville, E., Xiao, Y., Nouaille, S., Le Bourgeois, P., Heux, S., Portais, J. C., Monsan, P., Martens, E. C., Potocki-Veronese, G., et al. (2016). Functional characterization of a gene locus from an uncultured gut Bacteroides conferring xylo-oligosaccharides utilization to Escherichia coli. Mol Microbiol 102, 579-592.
Terrapon, N., Lombard, V., Drula, E., Lapebie, P., Al-Masaudi, S., Gilbert, H. J., and Henrissat, B. (2018). PULDB: the expanded database of Polysaccharide Utilization Loci. Nucleic Acids Res 46, D677-D683.
Thibault, J. F. (1979). Automatisation du dosage des substances pectiques par la méthode au métahydroxydiphényle. Lebensml Wiss Technol 12, 247-251.
Ting, L., Cowley, M. J., Hoon, S. L., Guilhaus, M., Raftery, M. J., and Cavicchioli, R. (2009). Normalization and statistical analysis of quantitative proteomics data generated by metabolic labeling. Mol Cell Proteomics 8, 2227-2242
Wu, M., McNulty, N. P., Rodionov, D. A., Khoroshkin, M. S., Griffin, N. W., Cheng, J., Latreille, P., Kerstetter, R. A., Terrapon, N., Henrissat, B., et al. (2015). Genetic determinants of in vivo fitness and diet responsiveness in multiple human gut Bacteroides. Science 350, aac5992.
Zeevi, D., Korem, T., Zmora, N., Israeli, D., Rothschild, D., Weinberger, A., Ben-Yacov, O., Lador, D., Avnit-Sagi, T., Lotan-Pompan, M., et al. (2015). Personalized Nutrition by Prediction of Glycemic Responses. Cell 163, 1079-1094.
Zhao, L., Zhang, F., Ding, X., Wu, G., Lam, Y. Y., Wang, X., Fu, H., Xue, X., Lu, C., Ma, J., et al. (2018). Gut bacteria selectively promoted by dietary fibers alleviate type 2 diabetes. Science 359, 1151-1156.

### Example 1—Glycan-Coated Magnetic Beads

A food-grade, pea fiber preparation was purchased from a commercial supplier. The compositional analysis of the pea fiber preparation is found in Table A. Wheat Arabinoxylan and Icelandic Moss Lichenan were purchased from Megazyme (P-WAXYL, P-LICHN) and yeast alpha-mannan was purchased from Sigma-Aldrich (M7504). Polysaccharides were solubilized in water (at a concentration of 5 mg/mL for pea fiber and 20 mg/mL for arabinoxylan and lichenan), sonicated and heated to 100° C. for 1 minute, then centrifuged at 24,000×g for 10 minutes to remove debris. TFPA-PEG3-biotin (Thermo Scientific), dissolved in DMSO (10 mg/mL) was added to the polysaccharide solution at a ratio of 1:5 (v/v). The sample was subjected to UV irradiation for 10 minutes (UV-B 306 nm, 7844 mJ total), and then diluted 1:4 to facilitate desalting on 7 kD Zeba spin columns (Thermo Scientific).

Biotinylated polysaccharide was mixed with one of several biotinylated fluorophores (PF-505, PF-510LSS, PF-633, PF-415; all at a concentration of 50 ng/mL; all obtained from Promokine). A 500 μL aliquot of this preparation was incubated with 10⁷ paramagnetic streptavidin-coated silica beads (LSKMAGT, Millipore Sigma) for 24 hours at room temperature. Beads were washed by centrifugation three times with 1 mL HNTB buffer (10 mM HEPES, 150 mM NaCl, 0.05% Tween-20, 0.1% BSA) followed by addition of 5 μg/mL streptavidin (Jackson Immunoresearch) in HNTB (30 min incubation at room temperature). Beads were washed as before and then incubated with 250 μL of the biotinylated polysaccharide preparation. The washing, streptavidin, and polysaccharide incubation steps were repeated three times.

Bead preparations were assessed using an Aria III cell sorter (BD Biosciences) to confirm adequate labeling. Beads were incubated with 70% ethanol for 1 minute in a biosafety cabinet, then washed three times with 1 mL sterile HNTB using a magnetic stand. The different bead types were combined, diluted, and aliquoted to 10⁷ beads per 650 μL HNTB in sterile Eppendorf microcentrifuge tubes. The number of beads in each aliquot was counted using an Aria III cell sorter and CountBright fluorescent microspheres (BD Bioscience).

Bead preparations were analyzed by GC-MS to quantify the amount of carbohydrate bound. Beads were sorted back into their polysaccharide types based on fluorescence using an Aria III sorter (average sort purity, 96%). Sorted samples were centrifuged (500×g for 5 minutes) to pellet beads and the beads were transferred to a 96-well plate. All bead samples were incubated with 1% SDS/6M Urea/HNTB for 10 minutes at room temperature to remove exogenous components, washed three times with 200 μL HNTB using a magnetic plate rack, and then stored overnight at 4° C. prior to monosaccharide analysis. The number and purity of beads in each sorted sample was determined by taking an aliquot for analysis on the Aria III cell sorter. Equal numbers of beads from each sample were transferred to a new 96-well plate and the supernatant was removed with a magnetic plate rack. For acid hydrolysis, 200 μL of 2M trifluoroacetic acid and 250 ng/mL myo-inositol-D6 (CDN Isotopes; spike-in control) were added to each well, and the entire volume was transferred to 300 μL glass vials (ThermoFisher; catalog number C4008-632C). Another aliquot was taken to verify the final number of beads in each sample. Monosaccharide standards were included in separate wells and subjected to the hydrolysis protocol in parallel with the other samples. Vials were crimped with Teflon-lined silicone caps (ThermoFisher) and incubated at 100° C. with rocking for 2 h. Vials were then cooled, spun to pellet beads, and their caps were removed. A 180 μL aliquot of the supernatant was collected and transferred to new 300 μL glass vials. Samples were dried in a SpeedVac for 4 hours, methoximated in 20 μL O-methoxyamine (15 mg/mL pyridine) for 15 h at 37° C., followed by trimethylsilylation in 20 μL MSTFA/TMCS [N-Methyl-N-trimethylsilyltrifluoroacetamide/2,2,2-trifluoro-N-methyl-N-(trimethylsilyl)-acetamide, chlorotrimethylsilane] (ThermoFisher) for 1 h at 70° C. One half volume of heptane (20 μL) was added before loading the samples for injection onto a 7890B gas chromatography system coupled to a 5977B MS detector (Agilent). The mass of each monosaccharide detected in each sample of sorted beads was determined using monosaccharide standard curves. This mass was then divided by the final count of beads in each sample to produce a measurement of mass of recoverable monosaccharide per bead.

TABLE 1
Monosaccharide analysis of wheat arabinoxylan
beads and pea fiber beads

| | | Mean (pg/bead) | sd |
|---|---|---|---|
| Xylose | Arabinoxylan beads | 0.17 | 0.12 |
| | Pea Fiber beads | 0.06 | 0.07 |
| | Uncoated beads | 0.01 | 0.01 |
| Arabinose | Arabinoxylan beads | 0.54 | 0.25 |
| | Pea Fiber beads | 0.2 | 0.06 |
| | Uncoated beads | 0.06 | 0.02 |
| Mannose | Arabinoxylan beads | 0.02 | 0.02 |
| | Pea Fiber beads | 0.04 | 0.02 |
| | Uncoated beads | 0.06 | 0.03 |
| Galactose | Arabinoxylan beads | 0.02 | 0.01 |
| | Pea Fiber beads | 0.05 | 0.03 |
| | Uncoated beads | 0 | 0.01 |
| Glucose | Arabinoxylan beads | 0.02 | 0.04 |
| | Pea Fiber beads | 0.01 | 0.02 |
| | Uncoated beads | 0.01 | 0.01 |

### Example 2—In Vivo Screen for Fiber Preparations that Target Specific Human Gut Microbes

In the present study, we describe an in vivo approach for identifying fibers and their bioactive components that selectively increase the fitness of a group of human gut Bacteroides, and the different mechanisms these organisms deploy when encountering these nutrient resources and one another. The bacterial targets for fiber-based manipulation originated from our previous study of twins stably discordant for obesity (Ridaura et al., 2013). Fecal microbiota from these twin pairs transmitted discordant adiposity and metabolic dysfunction phenotypes to recipient germ-free mice. Co-housing mice shortly after they received microbial communities from lean (Ln) or obese (Ob) co-twins prevented recipients of the Ob donor microbiota from developing obesity and associated metabolic abnormalities. Analysis of their gut communities revealed that invasion of Bacteroides species from Ln into Ob microbiota, notably B. thetaiotaomicron, B. vulgatus, B. caccae, and B. cellulosilyticus, correlated with protection from the increased adiposity and metabolic phenotypes that developed in co-housed Ob-Ob controls. Invasion was diet-dependent, occurring when animals consumed a human diet designed to represent the lower tertile of consumption of saturated fats and upper tertile of consumption of fruits and vegetables (high in fiber) in the USA, but not when they consumed a diet representing the upper tertile of saturated fat and lower tertile of fruit and vegetable consumption (Ridaura et al., 2013). Here we identify dietary fiber preparations and constituent bioactive components that increase the fitness of these targeted Bacteroides (B. thetaiotaomicron, B. vulgatus, B. caccae, and/or B. cellulosilyticus) in vivo in the high saturated fatty acid-low fruits and vegetables (HiSF-LoFV) diet context. To do so, we first colonized germ-free mice with a defined consortium of sequenced bacterial strains cultured from a Ln donor in an obesity-discordant twin pair. Mice were fed 144 different diets generated by supplementing the HiSF-LoFV formulation with 34 different food-grade fiber preparations in different combinations at different concentrations. Armed with a consortium that contained targeted Bacteroides species, each in the form of a library of tens of thousands of transposon (Tn) mutant strains, and employing high resolution mass spectrometry, we subsequently characterized the effects of monotonous feeding of selected fiber preparations on the community's expressed proteome and on the fitness of Tn mutants. By identifying polysaccharide processing genes whose expression was increased and that functioned as key fitness determinants, we inferred which components of the fiber preparations were bioactive. Time series proteomic analyses of the complete community and derivatives lacking one or more Bacteroides, revealed nutrient harvesting strategies resulting in, as well as alleviating interspecies competition for fiber components. Finally, administering artificial food particles coated with dietary polysaccharides to gnotobiotic mice with deliberately varied community membership further established the contributions of individual Bacteroides species to glycan processing in vivo.

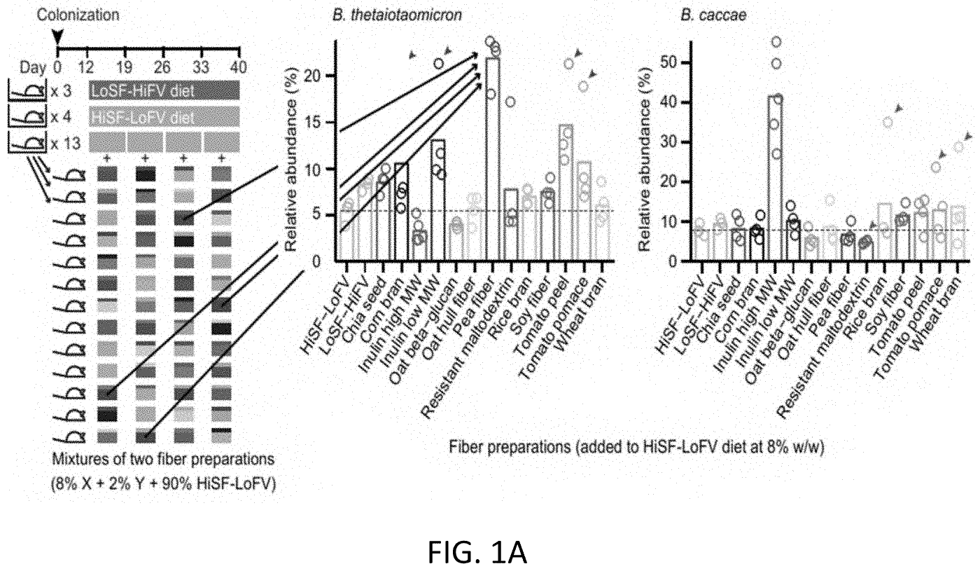

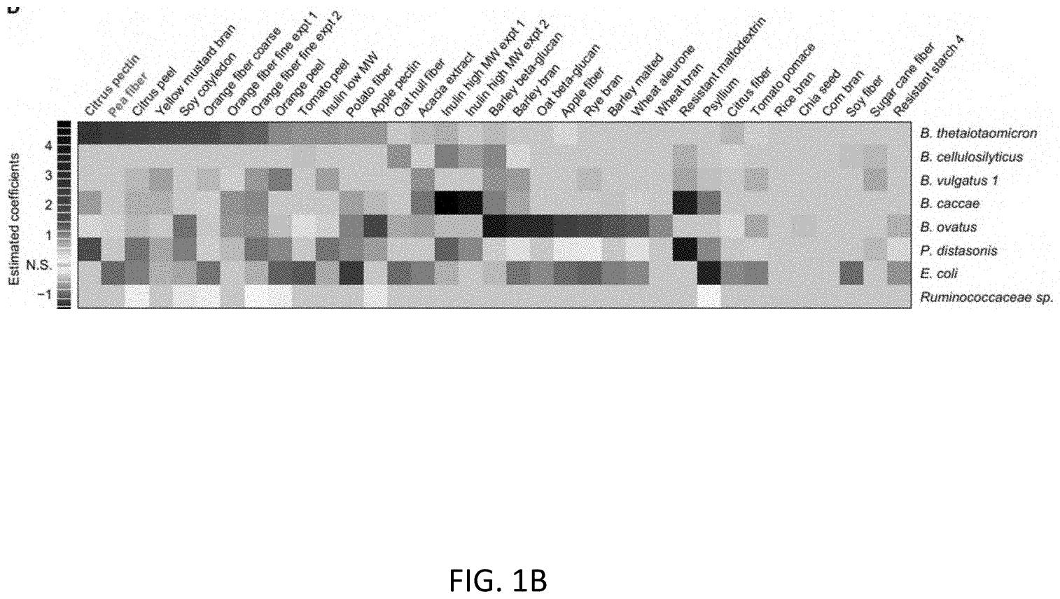

A schematic of the experimental design for screening 34 food grade fibers is shown in FIG. 1A. In total, three separate experiments were performed to complete an analysis of the effects of these fiber preparations on community structure. These fibers were obtained from diverse plant sources including fruits, vegetables, legumes, oilseeds, and cereals. Ten to 13 different fibers were tested per experiment (Table 2). Each mouse was colonized with a 20-member consortium of sequenced bacterial strains cultured from a single Ln co-twin donor. Each animal received a different fiber-supplemented diet each week for a total of four weeks. Each of the 144 unique diets tested contained one fiber type present at a concentration of 8% (w/w) and another fiber type at 2%. These two concentrations were systematically paired (Methods) to maximize the number of fiber preparations tested (FIG. 1A). Moreover, fiber types were presented in varying orders during the diet oscillation, mitigating potential hysteresis effects. Control groups were monotonously fed the unsupplemented HiSF-LoFV or LoSF-HiFV diet.

TABLE 2
Food-grade dietary fiber preparations
(A) Experiment details

| | Screening | | Screening |
|---|---|---|---|
| Fiber preparation | experiment used | Fiber preparation | experiment used |
| Citrus pectin | 3 | Oat beta-glucan | 2 |
| Pea fiber | 2 | Apple fiber | 1 |
| Citrus peel | 3 | Rye bran | 1 |
| Yellow mustard | 3 | Barley malted | 1 |
| Soy cotyledon | 3 | Wheat aleurone | 1 |
| Orange fiber (Coarse) | 1 | Wheat bran | 2 |
| Orange fiber (Fine) | 1 and 3 | Resistant | 2 |
| | | maltodextrin | |
| Orange peel | 3 | Psyllium | 3 |
| Tomato peel | 2 | Cocoa | 3 |
| Inulin, LMW | 2 | Citrus fiber | 3 |
| Potato Fiber | 3 | Tomato pomace | 2 |
| Apple pectin | 1 | Rice bran | 2 |
| Oat hull fiber | 2 | Chia seed | 2 |
| Acacia extract | 1 | Corn bran | 2 |
| Inulin, HMW | 2 and 3 | Soy fiber | 2 |
| Barley beta-glucan | 1 | Sugar cane fiber | 3 |
| Barley bran | 1 | Resistant starch 4 | 3 |

(B) Compositional analysis

| | % | % | % | HMW | LMW | % | % | % | % | % |
|---|---|---|---|---|---|---|---|---|---|---|
| | TDF | IDF | SDF | DF | DF | Prot | Fat | Carb | Moisture | Ash |
| Citrus | 78.9 | 1.4 | 75.5 | 76.9 | 2 | 3.34 | 0.56 | 86.82 | 7.97 | 1.31 |
| pectin* | | | | | | | | | | |
| Pea fiber* | 67.2 | 61.36 | 4.94 | 66.3 | 0.8 | 9.49 | 0.93 | 79.75 | 7.37 | 2.46 |
| Citrus peel | 70.9 | 47.7 | 23.2 | 70.9 | 0.6 | 4.44 | 2.31 | 83.16 | 6.85 | 3.24 |
| Yellow | 41.8 | 40.7 | 0.47 | 40.8 | 1 | 25.34 | 10.68 | 50.86 | 8.12 | 5 |
| mustard | | | | | | | | | | |
| Soy | 62.9 | 54 | 7.5 | 61.5 | 1.4 | 24.49 | 1.48 | 60.78 | 8.41 | 4.84 |
| cotyledon | | | | | | | | | | |
| Orange | 68.5 | 33.2 | 29.5 | 68.5 | 0.6 | 7.47 | 2.16 | 80.92 | 5.69 | 1.96 |
| fiber | | | | | | | | | | |
| (Coarse)* | | | | | | | | | | |
| Orange | 68 | 28.2 | 28.1 | 66.8 | 1.1 | 9.92 | 4.13 | 78.39 | 4.74 | 1.17 |
| fiber (Fine) | | | | | | | | | | |
| Orange | 60.1 | 42.9 | 17.2 | 60.1 | 0.6 | 6.19 | 4.03 | 79.49 | 7.36 | 2.93 |
| peel | | | | | | | | | | |
| Tomato | 79.1 | 68.22 | 10.88 | 79.1 | 0.6 | 8.07 | 4.42 | 79.23 | 5.57 | 2.71 |
| peel | | | | | | | | | | |
| Inulin, | 98.5 | <0.5 | 98.5 | 86 | 12.5 | 0.4 | 1.18 | 95.14 | 3.2 | 0.08 |
| LMW | | | | | | | | | | |
| Potato | 65.5 | 53.9 | 9.9 | 63.8 | 1.7 | 7.28 | 1.48 | 79.14 | 9.41 | 2.69 |
| Fiber | | | | | | | | | | |
| Apple | 60 | 0.47 | 58.65 | 59.3 | 0.7 | 12.04 | 0.98 | 70.61 | 10.76 | 5.61 |
| pectin | | | | | | | | | | |
| Oat hull | 95.7 | 92.86 | 2.84 | 95.7 | 0.6 | 0.35 | 0.15 | 94.3 | 3.91 | 1.29 |
| fiber | | | | | | | | | | |
| Acacia | 72.4 | 0.47 | 72.4 | 72.4 | 0.6 | 0.79 | 0.65 | 84.11 | 9.89 | 4.56 |
| extract | | | | | | | | | | |
| Inulin, | 90.9 | ND | ND | 59.5 | 31.3 | 0.28 | 3.71 | 91.44 | 4.28 | 0.29 |
| HMW* | | | | | | | | | | |
| Barley | 84.6 | 0.47 | 74.4 | 81.6 | 3 | 3.08 | 1.56 | 88.45 | 5.85 | 1.06 |
| beta- | | | | | | | | | | |
| glucan | | | | | | | | | | |
| Barley | 46 | 11.1 | 20.8 | 45.2 | 0.9 | 18.72 | 4.13 | 69.28 | 5.69 | 1.96 |
| bran | | | | | | | | | | |
| (barley | | | | | | | | | | |
| fiber)* | | | | | | | | | | |
| Oat beta- | 46.6 | 25.6 | 20.3 | 45.5 | 1.1 | 21.64 | 4.98 | 65.45 | 4.07 | 3.86 |
| glucan | | | | | | | | | | |
| Apple fiber | 73.3 | 57.25 | 7.01 | 73.3 | 0.6 | 9.78 | 1.57 | 81.77 | 4.98 | 1.9 |
| Rye bran | 45.5 | 32.7 | 0.47 | 41.5 | 4 | 13.58 | 4.8 | 70.01 | 6.48 | 5.13 |
| Barley | 42.2 | 39.5 | 0.47 | 41.1 | 1.1 | 16.89 | 10.53 | 63.52 | 6.15 | 2.91 |
| malted | | | | | | | | | | |
| Wheat | 43.7 | 39.89 | 0.47 | 42.3 | 1.5 | 13.64 | 9.05 | 63.55 | 7.14 | 6.62 |
| aleurone | | | | | | | | | | |
| Wheat | 30.2 | 24.54 | 3.46 | 28 | 2.2 | 14.06 | 5.08 | 67.12 | 9.7 | 4.04 |
| bran | | | | | | | | | | |
| Resistant | 72.3 | 0.47 | 1.8 | 1.8 | 70.5 | 0.71 | 0.08 | 95.52 | 3.77 | 0.04 |
| maltodextrin | | | | | | | | | | |
| Psyllium | 95.6 | 87.8 | 3.6 | 91.4 | 4.2 | 1.63 | 0.74 | 88.08 | 6.98 | 2.57 |
| Cocoa | 31.6 | 21.5 | 9.3 | 30.8 | 0.9 | 27.81 | 12.61 | 50.29 | 2.67 | 6.62 |
| Citrus fiber | 91 | 85.3 | 4.7 | 91 | 0.6 | 0.61 | 1.23 | 90.07 | 3.51 | 4.58 |
| Tomato | 56.7 | 49.1 | 7.6 | 56.7 | 0.6 | 15.63 | 14.37 | 62.26 | 4.76 | 2.98 |
| pomace | | | | | | | | | | |
| Rice bran | 23.5 | 22.19 | 0.61 | 22.8 | 0.7 | 15.13 | 21.62 | 49.88 | 5.21 | 8.16 |
| Chia seed | 40.8 | 39.17 | 1.63 | 40.8 | 0.6 | 22.07 | 36.91 | 30.67 | 5.62 | 4.73 |
| Corn bran | 76.8 | 72.34 | 4.46 | 76.8 | 0.6 | 4.97 | 4.08 | 83.9 | 6.09 | 0.96 |
| Soy fiber | 93.8 | 89.29 | 3.11 | 92.4 | TBD | 1.58 | 1.05 | 89.97 | 4.88 | 2.52 |
| Sugar | 95.6 | 90.6 | 5 | 95.6 | 0.6 | 0.12 | 0.15 | 93.36 | 6.11 | 0.38 |
| cane fiber | | | | | | | | | | |
| Resistant | 90.7 | 70.3 | 20.4 | 90.7 | 0.6 | 0.12 | 0.08 | 86.48 | 11.72 | 1.8 |
| starch 4 | | | | | | | | | | |

Abbreviations:
dietary fiber (DF),
total dietary fiber (TDF),
insoluble dietary fiber (IDF),
soluble dietary fiber (SDF),
high molecular weight (HMW),
low molecular weight (LMW),
protein (Prot),
carbohydrate (Carb),
not determined (ND)
*See Tables A-F for monosaccharide analysis and glycosyl linkage analysis

TABLE 3
Monosaccharide analysis of HiSF-LoFV diet

| | F1 (390 ug) | | F2 (490 ug) | | F3 (480 ug) | |
|---|---|---|---|---|---|---|
| Glycosyl residue | Mass (mg) | Mol % | Mass (mg) | Mol % | Mass (mg) | Mol % |
| Arabinose (Ara) | 3.1 | 29.6 | 2 | 3.4 | 7.7 | 12.8 |
| Rhamnose (Rha) | 0.1 | 0.4 | 0.1 | 0.1 | 0.7 | 1 |
| Fucose (Fuc) | n.d. | — | n.d. | — | 0.1 | 0.1 |
| Xylose (Xyl) | 3.4 | 32.9 | 3.9 | 6.7 | 14.4 | 24 |
| Galacturonic acid (GalA) | 0.1 | 0.6 | 0.2 | 0.3 | 2.4 | 3.1 |
| Mannose (Man) | 1.8 | 14.1 | 6 | 8.6 | 13.9 | 19.4 |
| Galactose (Gal) | 0.9 | 7.2 | 0.7 | 1.1 | 4.9 | 6.8 |
| Glucose (Glc) | 1.9 | 15.2 | 55.8 | 79.8 | 23.5 | 32.7 |
| Sum= | 11.2 | | 68.7 | | 67.5 | |

TABLE 4
Glycosyl-linkage analysis of HiSF-LoFV diet

| | | % detected linkage | | |
|---|---|---|---|---|
| | Deduced Linkage | F1 | F2 | F3 |
| Arabinose | (t-Araf) | 19.7 | 5.2 | 3.9 |
| | (2-Araf) | 1.4 | 0.3 | 1.9 |
| | (3-Araf) | 0.7 | 0.2 | 0.8 |
| | (4-Arap or 5-Araf) | 2.4 | 0.5 | 1.6 |
| Xylose | (t-Xyl) | 0.9 | 0.4 | 2.4 |
| | (4-Xyl) | 17.8 | 8.4 | 9.1 |
| | (2-Xyl) | 1.8 | 0.7 | 2.3 |
| | (2,4-Xyl) | 0.7 | 0.8 | 0.4 |
| | (3,4-Xyl) | 5.9 | 2 | 2.1 |
| | (2,3,4-Xyl) | 6.6 | — | — |
| Fucose | (t-Fuc) | — | — | 0.3 |
| Galactose | (t-Gal) | 1.3 | 0.5 | 2.4 |
| | (3-Gal) | 0.9 | — | — |
| | (2-Gal) | — | — | 0.6 |
| | (4-Gal) | 2.6 | 0.9 | 4.8 |
| | (6-Gal) | 0.4 | — | 0.3 |
| | (3,6-Gal) | 4.6 | 1.2 | 0.4 |
| Mannose | (t-Man) | 5.2 | 5.4 | 6.3 |
| | (2-Man) | 2.9 | 1.5 | 1.5 |
| | (3-Man) | 0.9 | 0.3 | 0.2 |
| | (4-Man) | 1.4 | 1.4 | 13 |
| | (2,4-Man) | 0.2 | — | — |
| | (4,6-Man) | — | — | 1 |
| | (3,6-Man) | 1.2 | — | 0.5 |
| | (2,6-Man) | 1.1 | 0.6 | 0.9 |
| Glucose | (t-Glc) | 2.9 | 4.6 | 2.4 |
| | (3-Glc) | 4.8 | 1.1 | 1.5 |
| | (6-Glc) | 0.8 | 0.7 | 0.6 |
| | (4-Glc) | 10 | 58 | 29.9 |
| | (3,4-Glc) | 0.5 | 1.2 | 0.3 |
| | (2,4-Glc) | 0.3 | 0.5 | 0.5 |
| | (4,6-Glc) | — | 3.6 | 7.8 |

We analyzed the relative abundance of each member of the defined community at two time points at the end of each diet treatment by collecting fecal samples and performing 16S rRNA gene sequencing. Binning the data according to the fiber preparation present at 8% concentration revealed potent and specific effects on distinct taxa (FIG. 1A). To analyze the independent effects of the two fiber preparations administered during each diet treatment, we generated a linear mixed-effects model for each bacterial taxon using the data from the last two days of consumption of each diet. The coefficient estimates in these models describe the slope of the predicted dose response curve for each fiber preparation's effect on each community member (Tables 5A, 6A, 7A). Twenty-one fiber preparations had significant estimated coefficients of >1 (with a coefficient of 1 indicating a 1% increase in the relative abundance of a bacterial species for every 1% increase in the concentration of the fiber preparation added to the HiSF-LoFV diet) (FIG. 1B). Large coefficients were observed in the B. thetaiotaomicron models for citrus pectin (2.6) and pea fiber (2.1). The B. ovatus models revealed pronounced effects of barley beta-glucan (3.9) and barley bran (3.1). Estimated coefficients for high molecular weight inulin (4.5, B. caccae model), resistant maltodextrin (3.8, P. distasonis model), and psyllium (3.4, E. coli model) were notable with 8% fiber administration driving the relative abundance of these community members from 10-20% to nearly 50%. Two of the fiber preparations tested (rice bran and corn bran) either had no detectable effect on the abundance of community members or produced estimated coefficients<0.5. High molecular weight inulin and an orange fiber preparation were tested across two separate experiments; the results established that the effects on the relative abundances of community members were reproducible (coefficients were highly correlated between these independent experiments, R²=0.96; Tables 5A, 6A, 7A). The even distributions of residuals around the fitted values in the models indicated that there were no pronounced threshold or saturation effects of these fiber preparations at the concentrations tested. For bacterial species that exhibited notable responses to fiber (at least one coefficient>1), the average R² value of the models was 0.82. We repeated our analyses using DNA yield from each fecal sample to estimate the absolute abundance of each organism as a function of fiber preparation. The estimated coefficients obtained from these two measures were highly correlated (R²=0.88) (Tables 5B, 6B, 7B). Together, results obtained from this screen illustrate the specificity of the effects of different types of dietary fiber on community configuration.

TABLE 5
Screening Experiment 1

| | | | | | | | | | | | |
|---|---|---|---|---|---|---|---|---|---|---|---|
| Taxonomy | OTU no. | 1 | 2 | 3 | 4 | 5 | 6 | 7 | 8 | 9 | 10 |

(A) Estimated Coefficients from linear
mixed effect models generated using reabundance

| | | | | | | | | | | | |
|---|---|---|---|---|---|---|---|---|---|---|---|
| Bacteroides | 848236 | 0.31 | 0.83 | 1.33 | 1.62 | — | −0.48 | — | — | — | 0.61 |
| thetaiotaomicron | | | | | | | | | | | |
| Bacteroides | 539126 | — | — | — | — | −0.3 | −0.8 | — | — | — | −0.37 |
| cellulosilyticus | | | | | | | | | | | |
| Bacteroides | 850870 | — | — | 0.42 | 0.61 | −0.7 | −0.71 | — | — | −0.49 | 0.89 |
| vulgatus | | | | | | | | | | | |
| Bacteroides caccae | 579112 | — | −0.5 | −0.73 | −0.35 | −0.63 | −0.88 | −0.4 | −0.33 | — | −0.94 |
| Bacteroides ovatus | 844958 | 2.13 | 1.83 | 0.84 | — | 3.12 | 3.89 | 1.41 | 1.32 | 1.78 | 0.81 |
| Parabacteroides | 846317 | 0.24 | −0.65 | −0.45 | −0.37 | −0.22 | −0.34 | — | −0.23 | −0.15 | — |
| distasonis | | | | | | | | | | | |
| Escherichia coli | 1111717 | −1 | — | — | −0.9 | −0.95 | −0.48 | −0.84 | −0.8 | −1.05 | −0.84 |
| Ruminococcaceae | 360801 | — | 0.2 | — | 0.14 | — | — | — | — | — | — |
| sp. | | | | | | | | | | | |
| Subdoligranulum | 364609 | −0.34 | −0.29 | — | — | — | −0.2 | — | — | — | — |
| variabile | | | | | | | | | | | |
| Collinsella | 1110606 | −0.18 | −0.16 | −0.19 | −0.16 | −0.15 | −0.09 | −0.14 | −0.12 | −0.12 | −0.13 |
| aerofaciens | | | | | | | | | | | |
| Bacteroides | 840832 | | | | | | | | | | |
| massiliensis | | | | | | | | | | | |
| Odoribacter | 210303 | −0.04 | −0.04 | −0.02 | −0.02 | −0.03 | −0.05 | −0.03 | −0.02 | −0.04 | −0.04 |
| splanchnicus | | | | | | | | | | | |
| Bacteroides | de novo | 0.03 | 0.04 | 0.05 | 0.03 | — | — | — | — | 0.02 | — |
| finegoldii | OTU | | | | | | | | | | |
| Peptococcus niger | 1135793 | −0.35 | −0.21 | −0.25 | −0.27 | −0.23 | −0.34 | −0.15 | −0.14 | −0.23 | — |
| Dorea longicatena | de novo | −0.47 | −0.66 | −0.59 | −0.57 | — | 0.31 | — | — | — | −0.32 |
| | OTU | | | | | | | | | | |

(B) Estimated Coefficients from linear mixed effect
models generated using DNA-scaled abundance.

| | | | | | | | | | | | |
|---|---|---|---|---|---|---|---|---|---|---|---|
| Bacteroides | 848236 | — | 1.15 | 1.64 | 1.54 | — | — | — | — | — | — |
| thetaiotaomicron | | | | | | | | | | | |
| Bacteroides | 539126 | — | — | — | — | — | — | — | — | — | −0.55 |
| cellulosilyticus | | | | | | | | | | | |
| Bacteroides | 850870 | — | — | 0.78 | 0.63 | — | — | — | — | — | — |
| vulgatus | | | | | | | | | | | |
| Bacteroides caccae | 579112 | — | — | −0.45 | — | — | −0.55 | — | — | — | −0.9 |
| Bacteroides ovatus | 844958 | 1.54 | 2.05 | 1.35 | — | 3.72 | 4.9 | 0.97 | 1.06 | 1.64 | — |
| Parabacteroides | 846317 | — | −0.4 | — | — | — | — | — | — | — | −0.37 |
| distasonis | | | | | | | | | | | |
| Escherichia coli | 1111717 | −0.84 | — | — | −0.55 | −0.42 | — | −0.62 | −0.63 | −0.72 | −1 |
| Ruminococcaceae | 360801 | — | 0.24 | — | 0.14 | — | — | — | — | — | — |
| sp. | | | | | | | | | | | |
| Subdoligranulum | 364609 | −0.28 | — | 0.26 | — | — | — | — | — | — | −0.27 |
| variabile | | | | | | | | | | | |
| Collinsella | 1110606 | −0.16 | −0.11 | −0.12 | −0.12 | −0.09 | — | −0.13 | −0.09 | −0.09 | −0.15 |
| aerofaciens | | | | | | | | | | | |
| Bacteroides | 840832 | | | | | | | | | | |
| massiliensis | | | | | | | | | | | |
| Odoribacter | 210303 | −0.03 | — | — | — | — | −0.03 | −0.02 | — | −0.03 | −0.05 |
| splanchnicus | | | | | | | | | | | |
| Bacteroides | de novo | 0.02 | 0.04 | 0.07 | 0.03 | 0.02 | — | — | — | 0.02 | — |
| finegoldii | OTU | | | | | | | | | | |
| Peptococcus niger | 1135793 | −0.29 | — | — | −0.18 | −0.1 | −0.19 | −0.13 | −0.12 | −0.15 | −0.19 |
| Dorea longicatena | de novo | −0.47 | −0.46 | −0.32 | −0.38 | — | 0.83 | — | — | — | −0.51 |
| | OTU | | | | | | | | | | |

1 - apple fiber,
2- apple pectin,
3 orange fiber fine,
4- orange fiber course,
5 barley bran,
5- barley beta glucan,
7- barley malted,
8- wheat aleurone,
9- rye bran,
10- acacia extract
“—” indicates estimate was not statistically significant (ANOVA, P < 0.05)
blank cells indicate that the organism was not detected above the 0.05% relative abundance cut-off in the experiment

TABLE 6
Screening Experiment 2
(A) Estimated Coefficients from linear mixed effect
models generated using relative abundance

| Taxonomy | OTU no. | 1 | 2 | 3 | 4 | 5 | 6 | 7 | 8 | 9 | 10 | 11 | 12 | 13 |
|---|---|---|---|---|---|---|---|---|---|---|---|---|---|---|
| Bacteroides | 848236 | 0.87 | — | — | 0.89 | — | — | — | — | — | — | 2.09 | — | — |
| thetaiotaomicron | | | | | | | | | | | | | | |
| Bacteroides | 539126 | — | 0.69 | − | 0.47 | — | — | — | −0.63 | — | 0.83 | — | — | 0.51 |
| cellulosilyticus | | | | | | | | | | | | | | |
| Bacteroides | 850870 | 0.9 | — | — | — | — | — | — | — | — | — | — | — | — |
| vulgatus | | | | | | | | | | | | | | |
| Bacteroides | 579112 | — | 4.54 | — | — | — | — | — | — | — | — | — | — | — |
| caccae | | | | | | | | | | | | | | |
| Bacteroides | 844958 | 0.35 | −0.55 | — | — | — | 0.52 | 0.91 | — | 2.99 | 0.68 | 0.34 | — | — |
| ovatus | | | | | | | | | | | | | | |
| Parabacteroides | 846317 | −1.06 | −0.95 | — | — | — | — | — | 3.75 | — | — | — | — | — |
| distasonis | | | | | | | | | | | | | | |
| Escherichia coli | 1111717 | — | — | −0.92 | −1.16 | — | — | — | — | −0.89 | −1.03 | −1.09 | −0.85 | −1.11 |
| Ruminococcaceae | 360801 | −0.03 | — | 0.03 | 0.09 | — | — | — | — | — | — | — | — | — |
| sp. | | | | | | | | | | | | | | |
| Subdoligranulum | 364609 | −0.28 | −0.4 | — | — | — | — | — | −0.26 | — | — | −0.33 | — | — |
| variabile | | | | | | | | | | | | | | |
| Collinsella | 1110606 | — | −0.3 | −0.32 | −0.33 | — | −0.27 | — | −0.34 | — | — | −0.34 | — | −0.3 |
| aerofaciens | | | | | | | | | | | | | | |
| Bacteroides | 840832 | −0.07 | −0.08 | −0.09 | −0.08 | −0.06 | — | −0.09 | −0.09 | −0.09 | — | −0.1 | — | −0.09 |
| massiliensis | | | | | | | | | | | | | | |
| Odoribacter | 210303 | — | −0.04 | — | — | — | — | — | −0.05 | — | −0.04 | −0.04 | — | — |
| splanchnicus | | | | | | | | | | | | | | |
| Bacteroides | de novo | | | | | | | | | | | | | |
| finegoldii | OTU | | | | | | | | | | | | | |
| Peptococcus | 1135793 | −0.21 | −0.19 | −0.18 | −0.18 | — | — | — | −0.27 | — | −0.18 | −0.28 | −0.21 | — |
| niger | | | | | | | | | | | | | | |

(B) Estimated Coefficients from linear mixed effect
models generated using DNA-scaled abundance.

| | | Screening experiment 2 | | | | | | | | | | | | |
|---|---|---|---|---|---|---|---|---|---|---|---|---|---|---|
| Taxonomy | OTU no. | 1 | 2 | 3 | 4 | 5 | 6 | 7 | 8 | 9 | 10 | 11 | 12 | 13 |
| Bacteroides | 848236 | 0.73 | — | — | — | — | — | — | — | — | — | 1.24 | − | — |
| thetaiotaomicron | | | | | | | | | | | | | | |
| Bacteroides | 539126 | — | −0.42 | — | — | — | — | — | −0.36 | — | — | — | — | — |
| cellulosilyticus | | | | | | | | | | | | | | |
| Bacteroides | 850870 | 0.67 | — | — | — | — | — | — | — | — | — | — | — | — |
| vulgatus | | | | | | | | | | | | | | |
| Bacteroides | 579112 | — | 1.96 | — | — | — | — | — | — | — | — | — | — | — |
| caccae | | | | | | | | | | | | | | |
| Bacteroides | 844958 | 0.39 | −0.36 | — | — | — | — | 0.5 | — | 2.01 | − | 0.27 | — | — |
| ovatus | | | | | | | | | | | | | | |
| Parabacteroides | 846317 | −0.55 | −0.53 | — | — | — | — | — | 1.72 | — | — | — | — | — |
| distasonis | | | | | | | | | | | | | | |
| Escherichia coli | 1111717 | 0.59 | −0.57 | −0.69 | −0.86 | −0.91 | — | — | −0.52 | — | −0.93 | −0.57 | −0.71 | −0.87 |
| Ruminococcaceae | 360801 | −0.02 | — | — | 0.04 | — | — | — | — | — | — | — | — | — |
| sp. | | | | | | | | | | | | | | |
| Subdoligranulum | 364609 | — | −0.22 | — | −0.14 | −0.19 | — | — | −0.17 | — | −0.18 | −0.16 | — | −0.17 |
| variabile | | | | | | | | | | | | | | |
| Collinsella | 1110606 | — | −0.19 | −0.25 | −0.23 | −0.26 | −0.23 | — | −0.22 | — | −0.18 | −0.2 | −0.2 | −0.27 |
| aerofaciens | | | | | | | | | | | | | | |
| Bacteroides | 840832 | −0.04 | −0.06 | −0.06 | −0.05 | −0.05 | −0.04 | −0.05 | −0.06 | −0.06 | −0.04 | −0.06 | −0.04 | −0.06 |
| massiliensis | | | | | | | | | | | | | | |
| Odoribacter | 210303 | — | −0.03 | — | — | — | — | — | −0.03 | — | −0.03 | −0.02 | — | −0.02 |
| splanchnicus | | | | | | | | | | | | | | |
| Bacteroides | de novo | | | | | | | | | | | | | |
| finegoldii | OTU | | | | | | | | | | | | | |
| Peptococcus | 1135793 | −0.11 | −0.13 | −0.14 | −0.13 | −0.15 | — | — | −0.17 | — | −0.15 | −0.16 | −0.15 | −0.14 |
| niger | | | | | | | | | | | | | | |

1, inulin LMW,
2, inulin HMW,
3- tomato pomace,
4- tomato peel,
5- rice bran,
6- chia see,
7- wheat bran,
8- resistant maltodextrin,
9- oat beta glucan,
10- oat hull fiber,
11- pea fiber,
12- corn bran
“—” indicates estimate was not statistically significant (ANOVA, P < 0.05)
blank cells indicate that the organism was not detected above the 0.05% relative abundance cut-off in the experiment

TABLE 7
Screening Experiment 3
(A) Estimated Coefficients from linear mixed effect
models generated using relative abundance

| | | Screening experiment 3 | | | | | | | | | | | |
|---|---|---|---|---|---|---|---|---|---|---|---|---|---|
| Taxonomy | OTU no. | 1 | 2 | 3 | 4 | 5 | 6 | 7 | 8 | 9 | 10 | 11 | 12 |
| Bacteroides | 848236 | 2 | — | — | 1 | — | 1.72 | 0.4 | 1.67 | 1.31 | −0.52 | 2.55 | 0.87 |
| thetaiotaomicron | | | | | | | | | | | | | |
| Bacteroides | 539126 | — | 0.57 | — | — | — | — | — | — | — | −0.88 | — | — |
| cellulosilyticus | | | | | | | | | | | | | |
| Bacteroides | 850870 | 0.6 | 0.73 | — | 1.09 | 0.55 | 0.81 | 0.65 | — | 0.82 | — | — | — |
| vulgatus | | | | | | | | | | | | | |
| Bacteroides | 579112 | −0.54 | — | — | — | −0.91 | −0.57 | — | — | −0.78 | 4.85 | −0.67 | 0.79 |
| caccae | | | | | | | | | | | | | |
| Bacteroides | 844958 | 0.59 | — | 0.64 | 0.52 | — | — | 0.72 | 1.17 | 0.93 | −0.46 | 0.34 | 1.05 |
| ovatus | | | | | | | | | | | | | |
| Parabacteroides | 846317 | −0.95 | −0.47 | −0.32 | −0.8 | −0.78 | −0.63 | −0.38 | −0.88 | −0.94 | −1.02 | −1.15 | −0.8 |
| distasonis | | | | | | | | | | | | | |
| Escherichia coli | 1111717 | −0.85 | — | −0.71 | −1.03 | 3.43 | −0.56 | −0.84 | −0.59 | −0.56 | −0.55 | — | −1.33 |
| Ruminococcaceae | 360801 | 0.17 | — | — | 0.13 | −0.08 | — | — | 0.21 | 0.07 | — | 0.45 | — |
| sp. | | | | | | | | | | | | | |
| Subdoligranulu | 364609 | −0.39 | −0.29 | — | — | −0.79 | −0.45 | — | — | −0.35 | −0.65 | −0.66 | −0.28 |
| m variabile | | | | | | | | | | | | | |
| Collinsella | 1110606 | −0.26 | — | — | −0.25 | −0.3 | — | — | — | −0.25 | — | −0.26 | — |
| aerofaciens | | | | | | | | | | | | | |
| Bacteroides | 840832 | −0.11 | −0.06 | — | −0.1 | −0.11 | −0.06 | −0.09 | −0.12 | −0.1 | −0.1 | −0.11 | −0.1 |
| massiliensis | | | | | | | | | | | | | |
| Odoribacter | 210303 | −0.04 | −0.04 | −0.02 | −0.03 | −0.07 | — | −0.04 | −0.04 | — | −0.05 | −0.04 | −0.02 |
| splanchnicus | | | | | | | | | | | | | |
| Bacteroides | de novo | 0.05 | — | — | 0.04 | — | 0.06 | 0.04 | 0.07 | 0.07 | — | 0.06 | 0.07 |
| finegoldii | OTU | | | | | | | | | | | | |
| Peptococcus niger | 1135793 | −0.42 | −0.34 | −0.37 | −0.41 | −0.58 | −0.38 | −0.24 | −0.53 | −0.37 | −0.5 | −0.49 | −0.48 |

1- citrus peel, 2- sugar cane fiber, 3- resistant starch 4, 4- orange peel, 5- psyllium, 6- yellow mustard bran, 7- citrus fiber, 8- soy
cotyledon, 9- orange fiber fine, 10- inulin HMW, 11- citrus pectin, 12- potato fiber
“—” indicates estimate was not statistically significant (ANOVA, P < 0.05)
blank cells indicate that the organism was not detected above the 0.05% relative abundance cut-off in the experiment

(B) Estimated Coefficients from linear mixed effect
models generated using DNA-scaled abundance.

| Taxonomy | OTU no. | 1 | 2 | 3 | 4 | 5 | 6 | 7 | 8 | 9 | 10 | 11 | 12 | 13 |
|---|---|---|---|---|---|---|---|---|---|---|---|---|---|---|
| Bacteroides | 848236 | 2.02 | — | — | 1.21 | − | 1.52 | — | 2.47 | 1.39 | −0.57 | 3.22 | 0.73 | 2.02 |
| thetaiotaomicron | | | | | | | | | | | | | | |
| Bacteroides | 539126 | — | — | — | — | −0.47 | — | −0.52 | — | — | −0.83 | 0.49 | — | — |
| cellulosilyticus | | | | | | | | | | | | | | |
| Bacteroides | 850870 | 0.62 | — | — | 1.26 | — | 0.64 | — | — | 0.91 | — | — | — | 0.62 |
| vulgatus | | | | | | | | | | | | | | |
| Bacteroides | 579112 | — | — | — | — | −0.92 | −0.51 | −0.51 | — | −0.57 | 4.22 | — | 0.76 | — |
| caccae | | | | | | | | | | | | | | |
| Bacteroides | 844958 | 0.67 | — | — | 0.66 | — | — | — | 1.76 | 1.01 | −0.48 | 0.65 | 0.94 | 0.67 |
| ovatus | | | | | | | | | | | | | | |
| Parabacteroides | 846317 | −0.76 | −0.6 | −0.36 | −0.64 | −0.82 | −0.54 | −0.67 | −0.51 | −0.73 | −0.9 | −0.89 | −0.65 | −0.76 |
| distasonis | | | | | | | | | | | | | | |
| Escherichia coli | 1111717 | −0.69 | −0.75 | −0.73 | −0.74 | 1.24 | — | −1.16 | — | — | — | — | −0.98 | −0.69 |
| Ruminococcaceae | 360801 | 0.17 | — | — | 0.15 | −0.1 | — | — | 0.28 | 0.08 | — | 0.53 | — | 0.17 |
| sp. | | | | | | | | | | | | | | |
| Subdoligranulum | 364609 | −0.25 | −0.34 | — | — | −0.7 | −0.37 | −0.32 | — | — | −0.56 | −0.46 | — | −0.25 |
| variabile | | | | | | | | | | | | | | |
| Collinsella | 1110606 | −0.21 | — | — | −0.2 | −0.29 | — | −0.19 | — | −0.2 | — | — | — | −0.21 |
| aerofaciens | | | | | | | | | | | | | | |
| Bacteroides | 840832 | −0.09 | −0.06 | — | −0.09 | −0.09 | — | −0.09 | −0.1 | −0.09 | −0.09 | −0.1 | −0.09 | −0.09 |
| massiliensis | | | | | | | | | | | | | | |
| Odoribacter | 210303 | −0.03 | −0.05 | −0.02 | −0.02 | −0.07 | — | −0.05 | — | — | −0.04 | — | — | −0.03 |
| splanchnicus | | | | | | | | | | | | | | |
| Bacteroides | de novo | 0.04 | — | — | 0.05 | — | 0.05 | — | 0.09 | 0.07 | — | 0.08 | 0.07 | 0.04 |
| finegoldii | OTU | | | | | | | | | | | | | |
| Peptococcus niger | 1135793 | −0.32 | −0.35 | −0.34 | −0.33 | −0.52 | −0.32 | −0.36 | −0.38 | −0.27 | −0.43 | −0.34 | −0.4 | −0.32 |

1, inulin LMW, 2, inulin HMW, 3- tomato pomace, 4- tomato peel, 5- rice bran, 6- chia see, 7- wheat bran,
8- resistant maltodextrin, 9- oat beta glucan, 10- oat hull fiber, 11- pea fiber, 12- corn bran
“—” indicates estimate was not statistically significant (ANOVA, P < 0.05)
blank cells indicate that the organism was not detected above the 0.05% relative abundance cut-off in the experiment

### Example 3: Proteomics and Forward Genetics Identify Bioactive Polysaccharides in Fiber Preparations

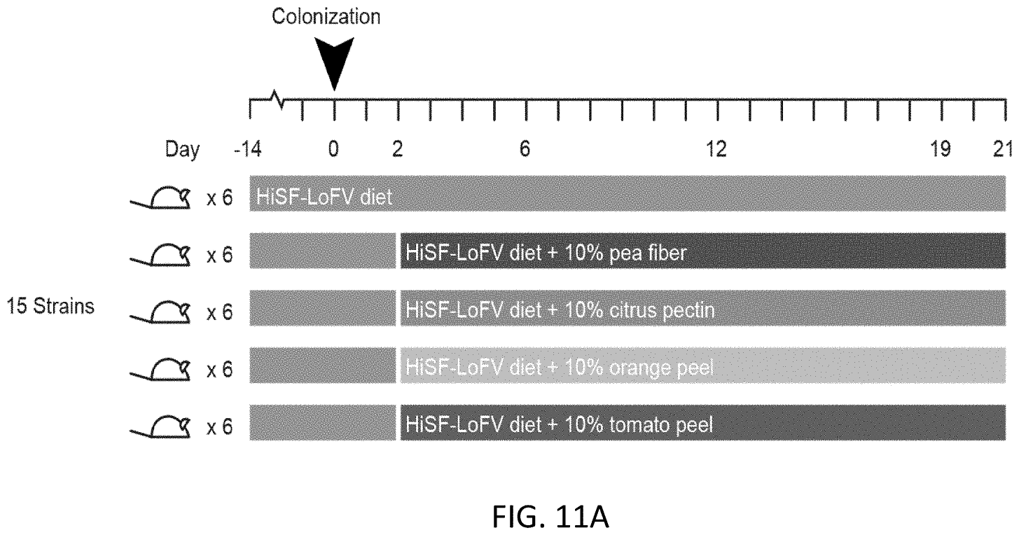

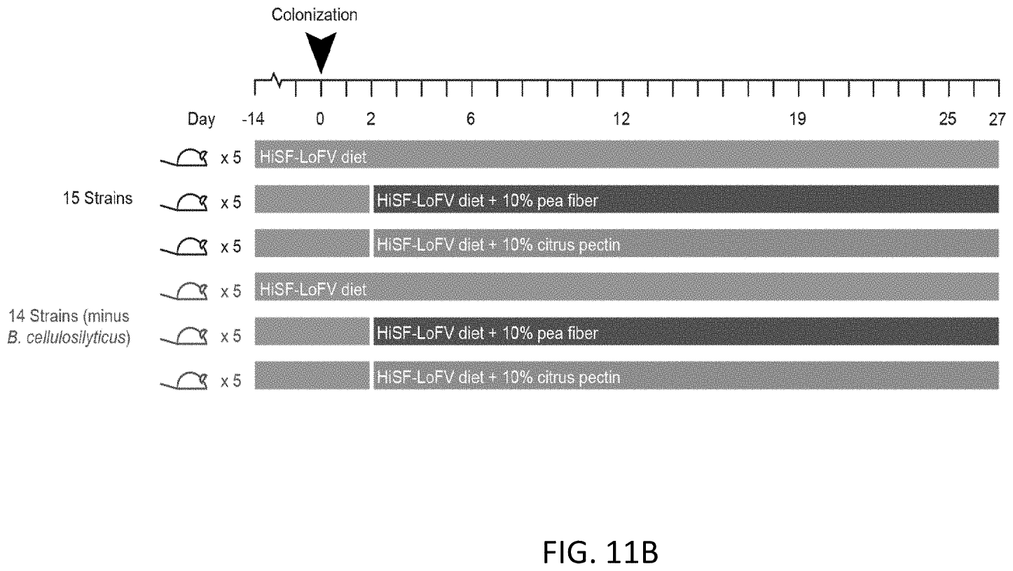

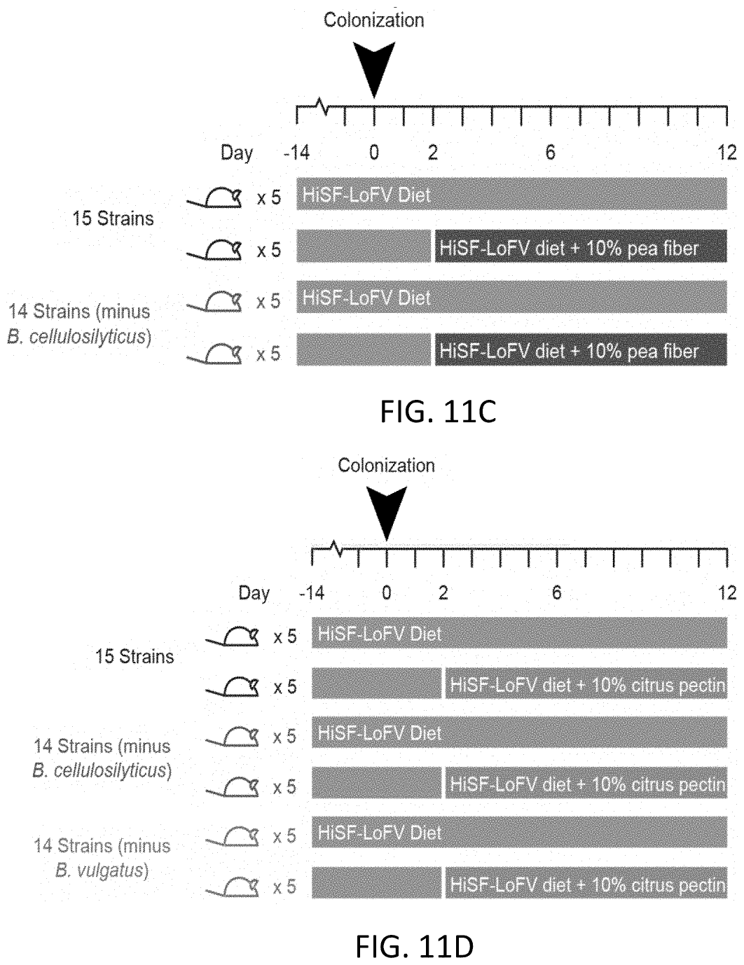

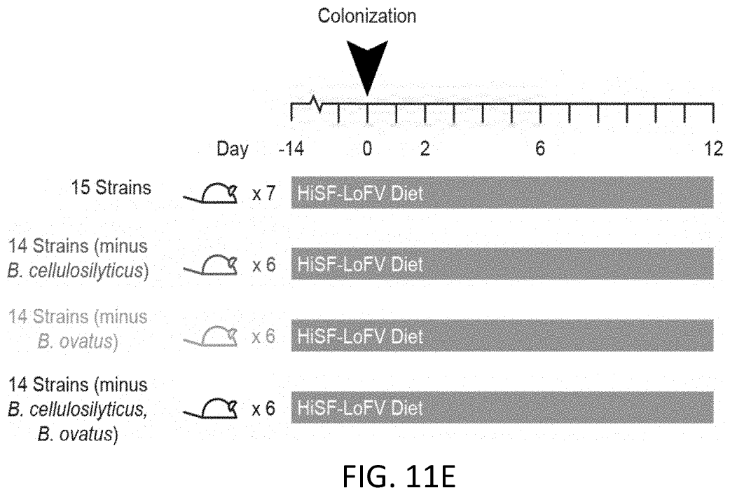

Several possible mechanisms could account for the increase of a target Bacteroides in response to fiber administration, including indirect effects involving other species. Therefore, we sought to determine which polysaccharides in the fiber preparations caused the target species to expand and whether they acted directly on those species by serving as nutrient sources for their growth. To do so, we simultaneously quantified community-wide protein expression and assessed the contributions of proteins to bacterial fitness using a forward genetic screen. The screen was based on genome-wide transposon (Tn) mutagenesis and a method known as multi-taxon INsertion Sequencing (INSeq), which allows simultaneous analysis of Tn mutant libraries generated from different Bacteroides species in the same recipient gnotobiotic mouse. We employed five INSeq libraries constructed using type strains corresponding to four Bacteroides species present in the Ln co-twin donor culture collection. The quality and performance of these libraries had been characterized previously in vitro and in vivo (30,300-167,000 isogenic Tn mutants/library; single site of Tn insertion/strain; 11-26 Tn insertions/gene; 71-92% genes covered/genome; (Hibberd et al., 2017; Wu et al., 2015)). Additionally, we simplified the community used in these experiments by omitting six strains from the original 20 member consortium that were not robust colonizers in the HiSF-LoFV diet context (Faith et al., 2014; Ridaura et al., 2013). All mice were colonized with the resulting 15-member community see Table S4 of Patnode et al., Cell, 2019, 179(1): 59-73) while consuming the base (unsupplemented) HiSF-LoFV diet. Animals were divided into five groups (n=6 animals/group) and were either continued on the base HiSF-LoFV diet or, two days after gavage, switched to the HiSF-LoFV diet supplemented with one of the fibers identified in the screen. We tested pea fiber, citrus pectin, orange peel, and tomato peel, each at a concentration of 10% (w/w), based on their ability to increase the representation of one or more of the targeted Bacteroides (FIG. 1B). All diets were administered ad libitum and given monotonously for the duration of the experiment (FIG. 11, see also Tables S4A-S4C of Patnode et al., Cell, 2019, 179(1): 59-73). DNA isolated from fecal samples was subjected to short read shotgun DNA sequencing (COmmunity PROfiling by Sequencing, COPRO-Seq; (Hibberd et al., 2017; McNulty et al., 2013) to quantify the representation of each community member as a function of fiber treatment, including the combined abundance of all INSeq mutants for a given species. Our previous studies had established that in aggregate, a population of INSeq mutants behaves similarly to the corresponding wild-type parental strain (Hibberd et al., 2017; Wu et al., 2015).

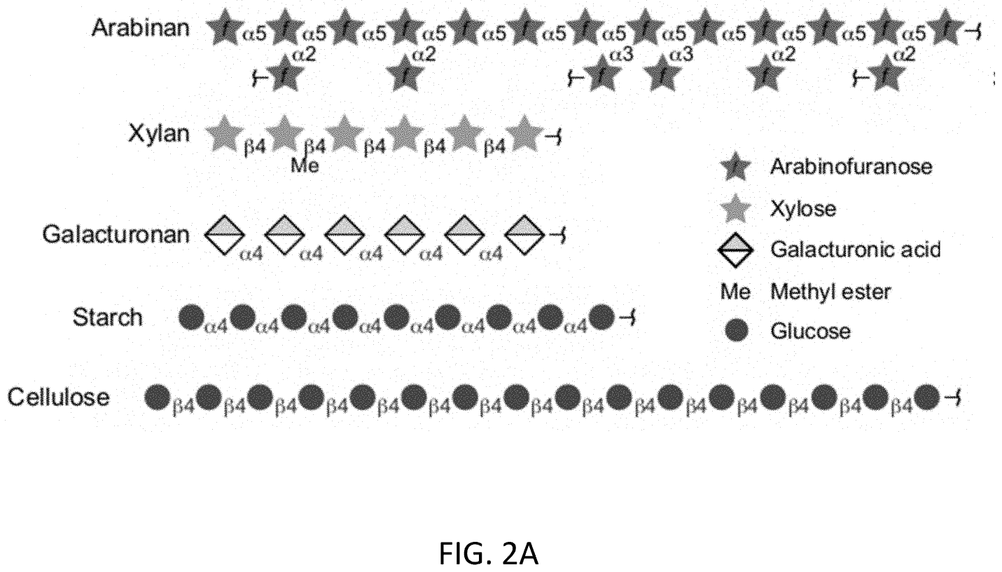

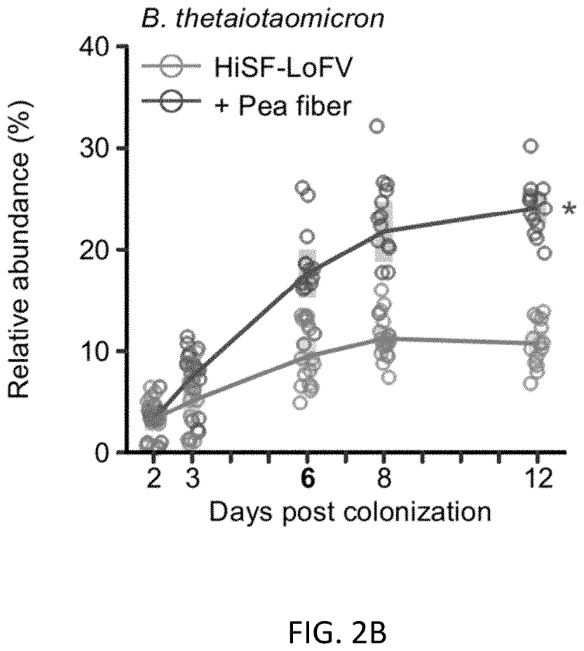

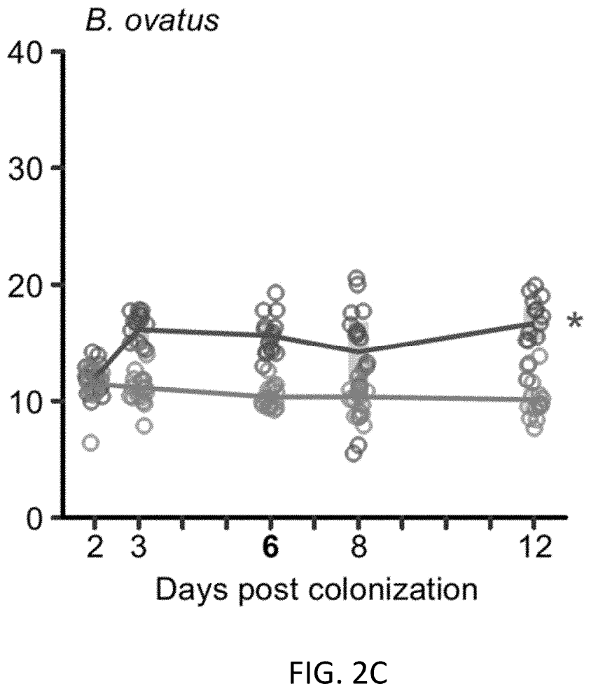

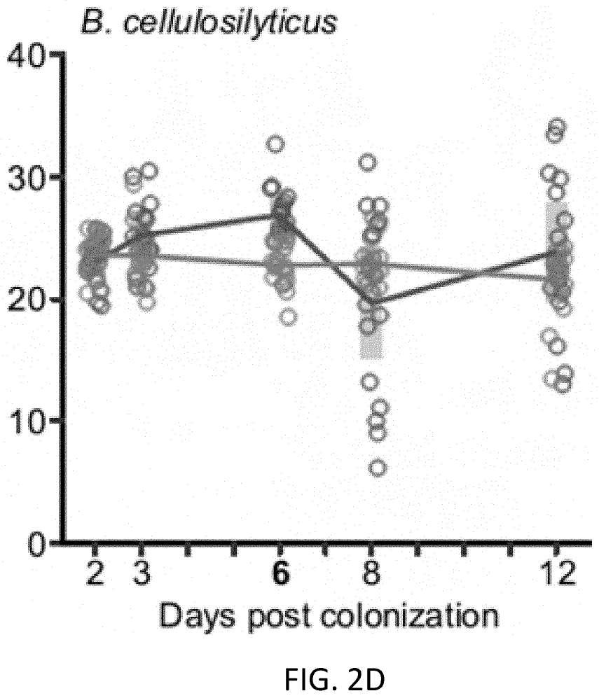

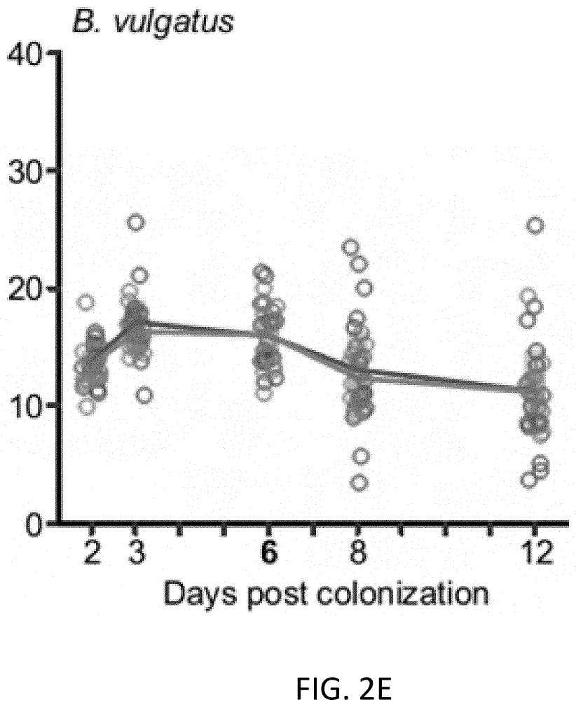

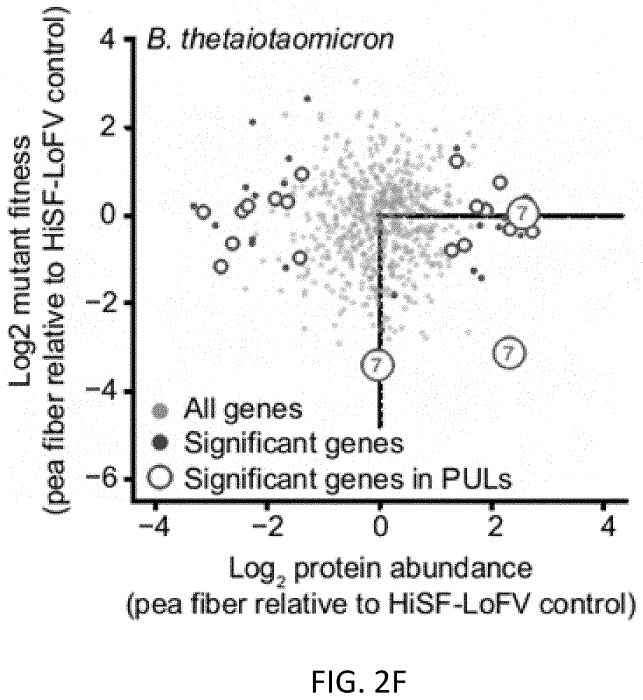

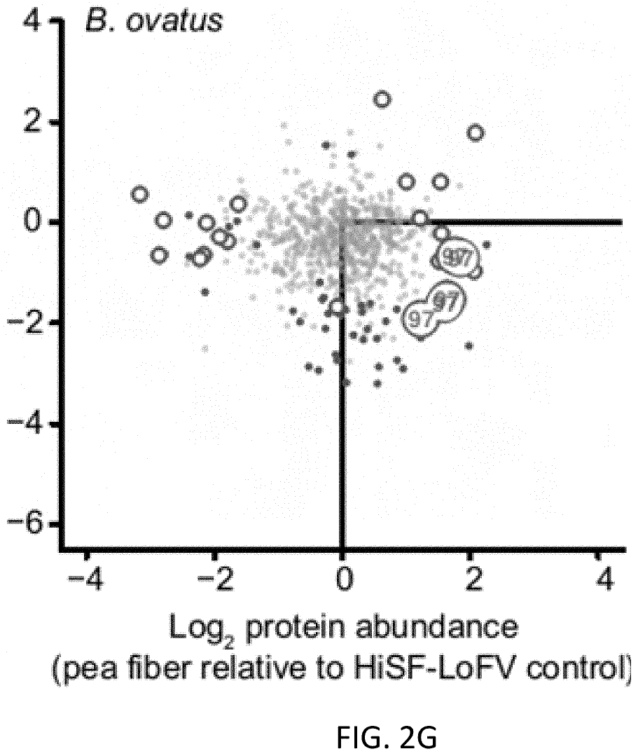

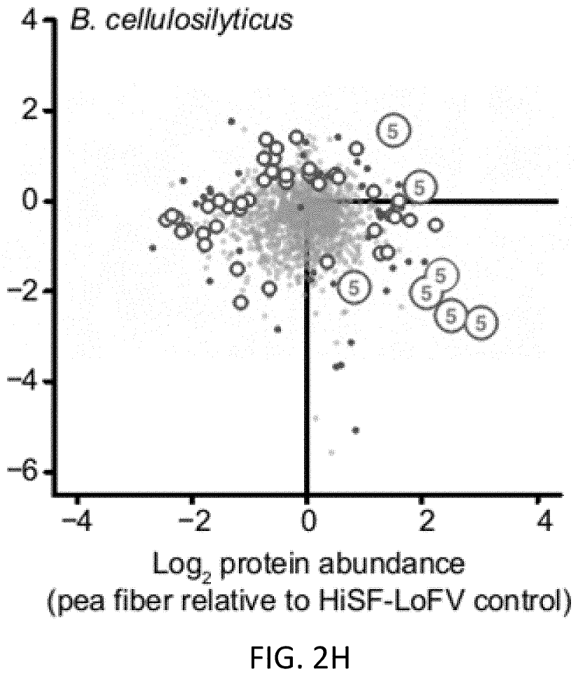

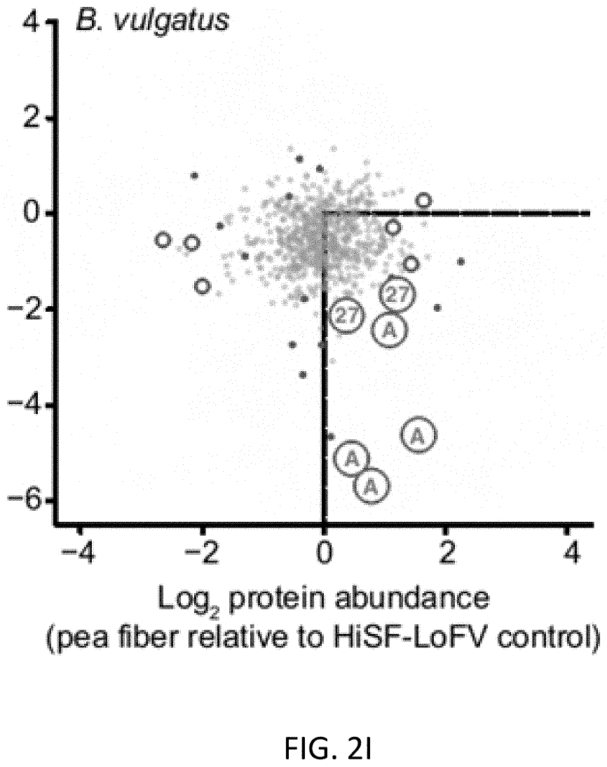

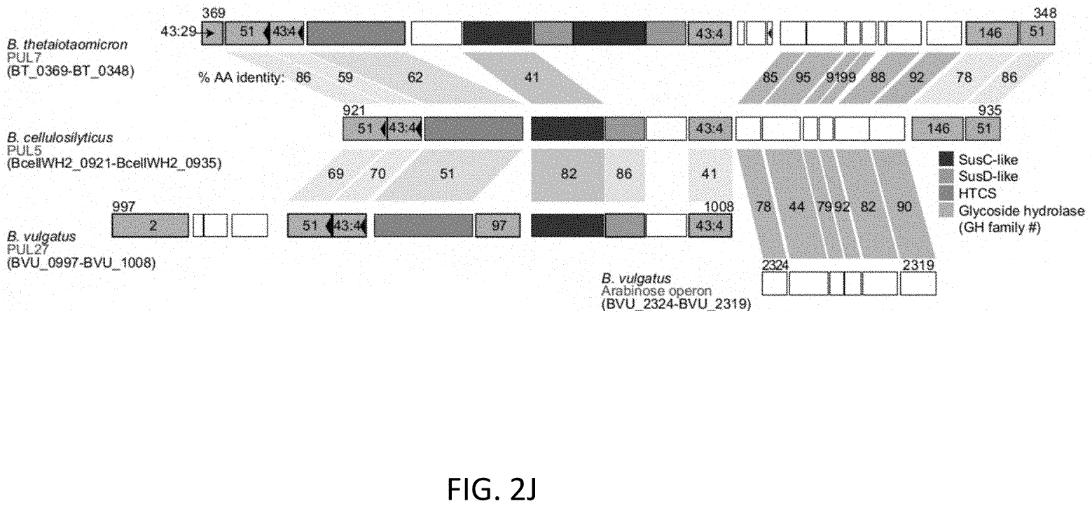

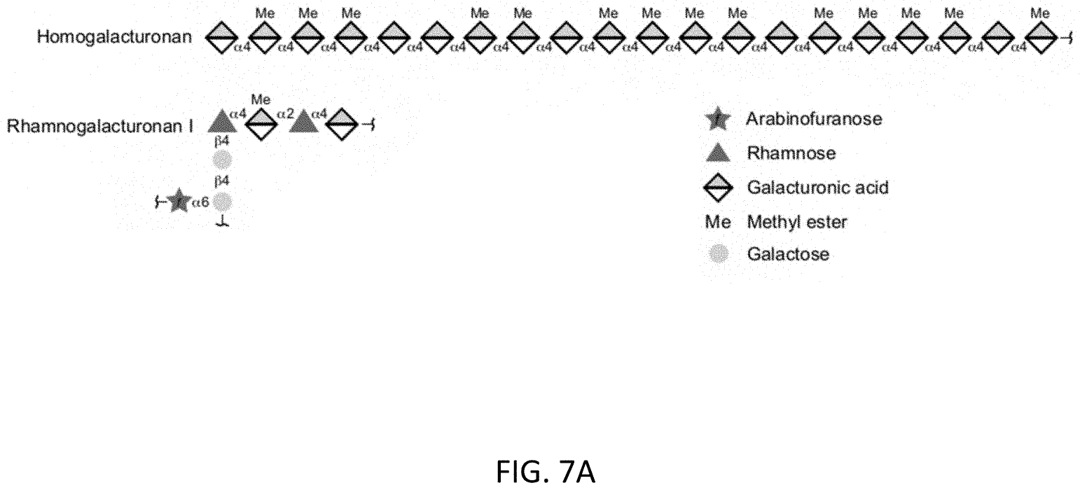

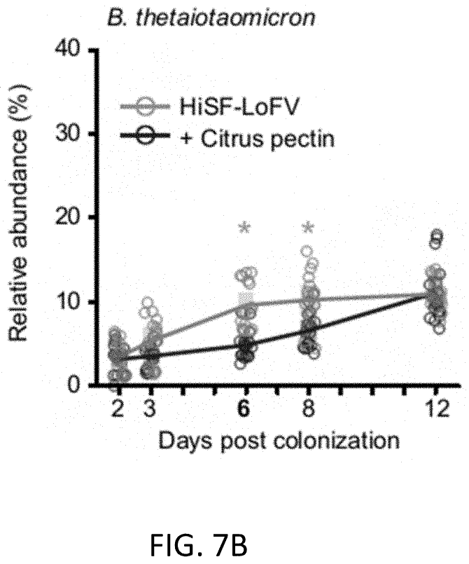

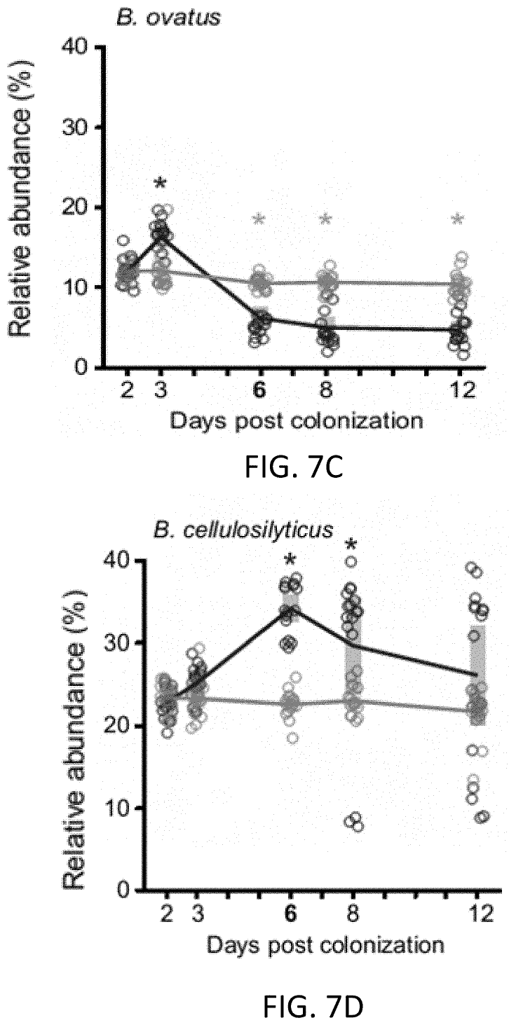

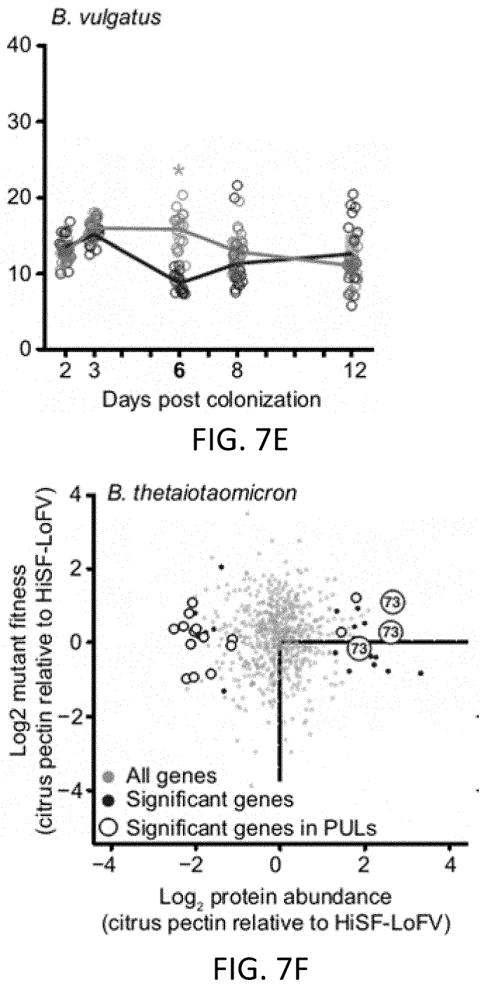

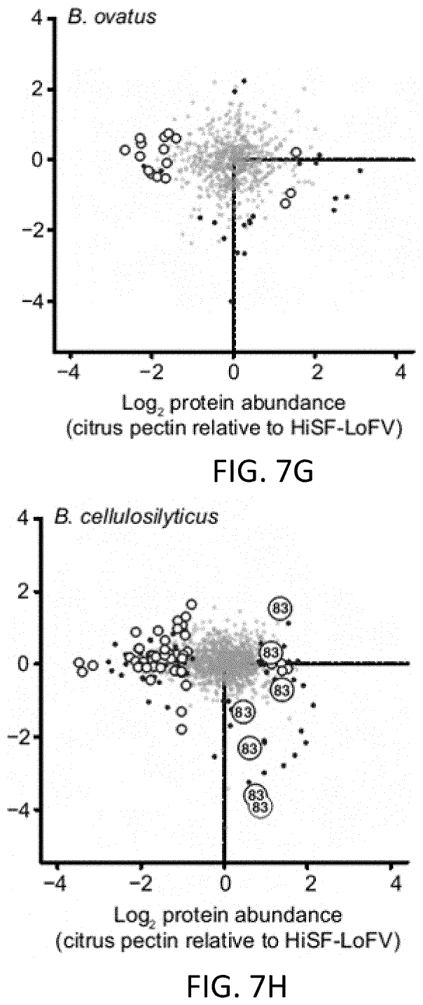

Consistent with results obtained from seven days of fiber administration in the screening experiments, we observed a statistically significant expansion of B. thetaiotaomicron VPI-5482 in mice consuming pea fiber (ANOVA, P<0.05; FIG. 2B, see also Tables S4A-S4C of Patnode et al., Cell, 2019, 179(1): 59-73). Also in accordance with observations made in the screen, the relative abundance of B. ovatus ATCC-8483 was significantly greater in the pea fiber-treated group (FIG. 2C), while B. cellulosilyticus WH2 and B. vulgatus ATCC-8482 did not exhibit significant changes during this time period (FIG. 2D and FIG. 2E). Citrus pectin induced significant expansion of three species (B. cellulosilyticus, Bacteroides finegoldii, and a member of the Ruminococcaceae) that was distinct from the set affected by pea fiber (FIG. 7D, see also Tables 54A, B, and D of Patnode et al., Cell, 2019, 179(1): 59-73). Although the fiber screen predicted an increase in the abundance of B. thetaiotaomicron in response to citrus pectin, this was not observed during monotonous feeding until later in the time course, indicating a difference between the strains employed or the effect of different community context (FIG. 7B). Orange peel significantly increased the representation of B. vulgatus, but otherwise had a minimal effect on community structure (see Table S4A of Patnode et al., Cell, 2019, 179(1): 59-73). Tomato peel did not significantly increase any members of this community, which may indicate the strain-dependency of a given species' response to a certain fiber when the effect size of a given fiber preparation is low (see Table S4A of Patnode et al., Cell, 2019, 179(1): 59-73). Since both pea fiber and citrus pectin had pronounced effects on distinct sets of taxa, we selected these preparations for more detailed functional studies of their utilization by community members.

Structural analyses of lead fibers—We used permethylation and gas-chromatography-mass spectrometry to analyze the monosaccharide composition and glycosidic linkages of polysaccharides present in pea fiber and citrus pectin. After accounting for starch (typically degraded and absorbed by the host) and cellulose (not metabolized by the target Bacteroides; (McNulty et al., 2013)), the most abundant polysaccharide in pea fiber was arabinan, consisting of a linear 1,5-linked arabinose backbone with arabinose residues as side chains at position 2 or 3 (FIG. 2A, Table C). Linear xylan (4-linked xylose), homogalacturonan (4-linked galacturonic acid) and rhamnogalacturonan I (2- and 2,4-linked rhamnose) were also detected as structural features of the polysaccharides in pea fiber. Homogalacturonan with a high degree of methyl esterification was the main structural component of citrus pectin (88.6% galacturonic acid), with arabinan, 1,4-linked galactan and RGI present as minor components (FIG. 7A, Table D).

High-resolution proteomic analysis of community gene expression—The results of these biochemical analyses raised the possibility that metabolism of arabinan in pea fiber and methylated homogalacturonan in citrus pectin were involved in the responses of target Bacteroides. To test this hypothesis, we turned to high-resolution shotgun proteomic analysis, focusing on fecal samples obtained on day 6 of the monotonous feeding experiment. After considering only peptides that uniquely mapped to a single seed protein, 11,493 proteins were advanced to quantitative analysis (summed abundances; 59% from community members, 36% from mouse and 2% from diet; see Methods). We calculated a z-score for each expressed protein from each bacterial species using the abundances of all proteins assigned to that individual species in a given sample. This allowed us to determine changes in the abundance of each protein irrespective of changes in the abundance of that species in the community. In the case of the Bacteroides species represented by INSeq libraries, we considered the measured abundance of a given protein to reflect the summed contributions of all the mutant strains of that species (thus representing the level of expression we would expect from a corresponding wild-type strain). Linear models were constructed using limma (Smyth, 2004; Ting et al., 2009) and significant effects were identified between bacterial protein abundances and supplementation of the control diet with pea fiber and citrus pectin (245 and 450 proteins, respectively; |fold-change|>log 2(1.2), P<0.05, FDR corrected). Bacteroides contain multiple polysaccharide utilization loci (PULs) in their genomes. PULs provide a fitness advantage by endowing a species with the ability to sense, import, and process complex glycans using their encoded carbohydrate-responsive transcription factors, SusC/SusD-like transporters, and carbohydrate active enzymes (CAZymes) (Glenwright et al., 2017; Kotarski and Salyers, 1984; Martens et al., 2011; McNulty et al., 2013; Shepherd et al., 2018). Eighty-five of the proteins whose levels were significantly altered by pea fiber and 134 that were significantly affected by citrus pectin were encoded by PULs (Terrapon et al., 2018).

Ranking proteins by the pea-fiber induced increase in their abundance disclosed that in B. thetaiotaomicron, 6 of the top 10 were encoded by PULs 7, 73, and 75. PUL7 is known to be involved in arabinan metabolism (Lynch and Sonnenburg, 2012; Schwalm et al., 2016), and encodes characterized and predicted arabinofuranosidases in glycoside hydrolase (GH) family 43, GH51, and GH146. PUL75 carries out the degradation of rhamnogalacturonan I (RGI) (Luis et al., 2018), but its expression is also triggered by exposure to purified arabinan in vitro (Martens et al., 2011). PUL73 processes homogalacturonan (Luis et al., 2018) and encodes CAZymes that cleave linked galacturonic acid residues and remove methyl and acetyl esters from galacturonic acid [polysaccharide lyase (PL)1, GH105, GH28, CE8, CE12 family members]. B. ovatus proteins encoded by predicted RGI-processing PULs (PUL97) (Luis et al., 2018) were among the most increased by pea fiber administration.

Supplementation of the HiSF-LoFV diet with citrus pectin resulted in increased abundance of proteins encoded by a B. cellulosilyticus PUL that is induced by homogalacturonan in vitro (PUL83). In addition, citrus pectin induced expression of proteins in several B. finegoldii PULs (PUL34, 35, 42, and 43) that encode galacturonan-processing enzymes (GH28, GH105, GH106, PL11 subfamily 1, CE8 and CE12). This latter finding correlates with the organism's citrus pectin-driven expansion (see Tables S4A-B of Patnode et al., Cell, 2019, 179(1): 59-73).

Combining proteomic and INSeq analyses—As noted above, we colonized mice with INSeq libraries and then fed them the base HiSF-LoFV diet for two days before switching the experimental groups to fiber-supplemented diets. We measured the abundances of Tn mutant strains, and calculated log ratios between fecal samples collected on experimental day 6 (posttreatment) and day 2 (pre-treatment); results were compared to the reference HiSF-LoFV treatment arm to focus on genes that had significant fitness effects in the context of these fibers (P<0.05, FDR corrected; see Methods; 223 genes, 24% in PULs; see also Table S6A of Patnode et al., Cell, 2019, 179(1): 59-73). Genes exhibiting a significant positive fold-change in protein abundance and negative effect on fitness when mutated appear in the bottom right quadrant of the orthogonal protein-fitness plots shown in FIG. 2F-FIG. 2I.

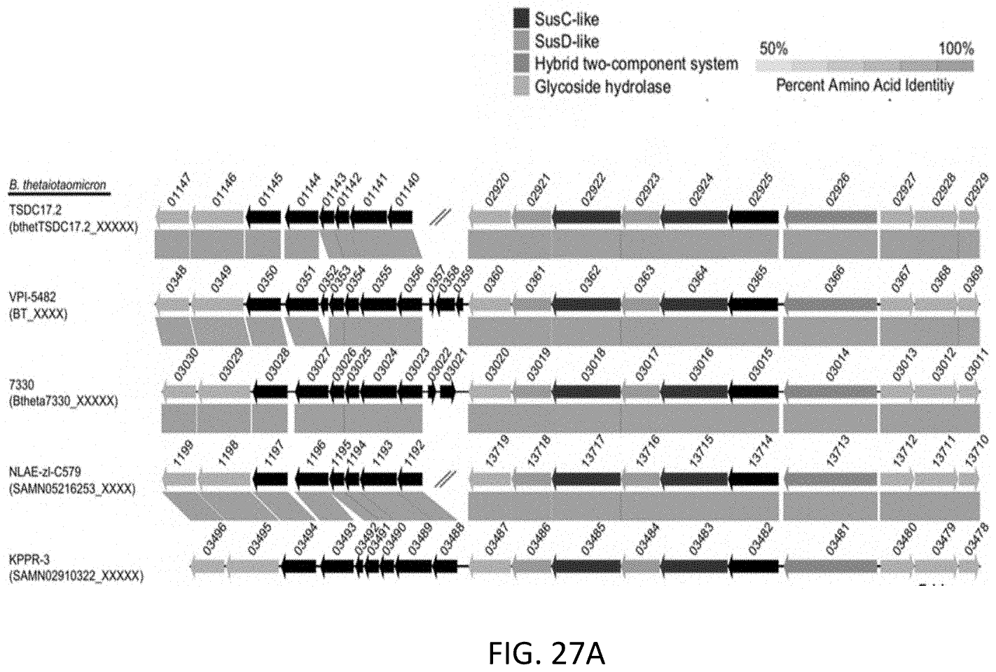

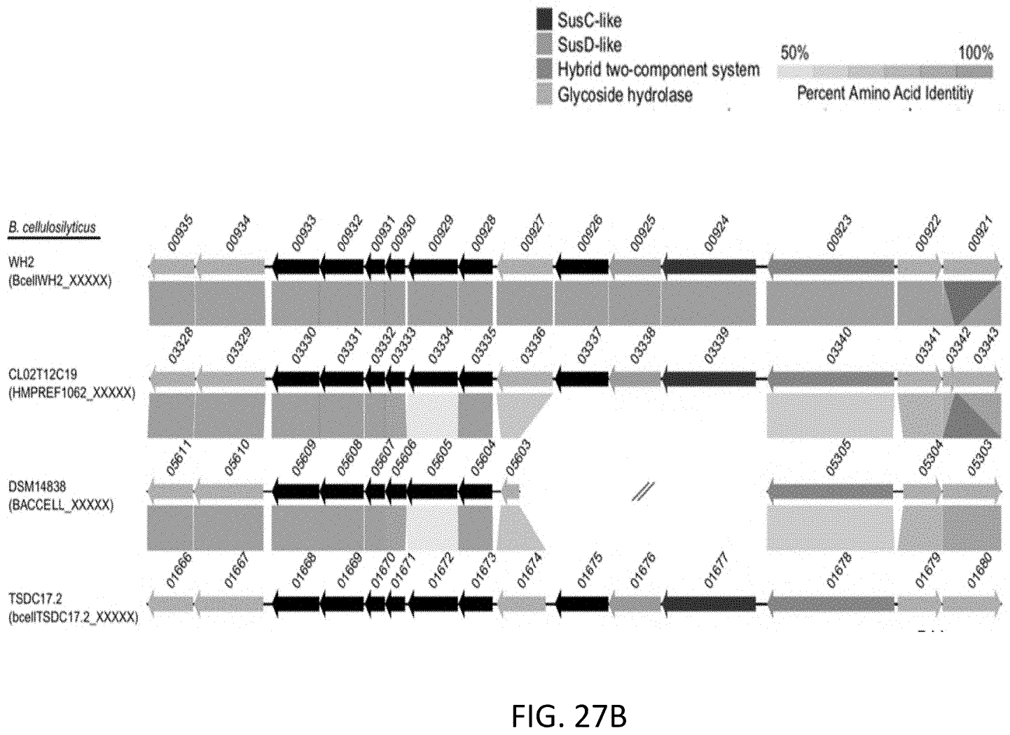

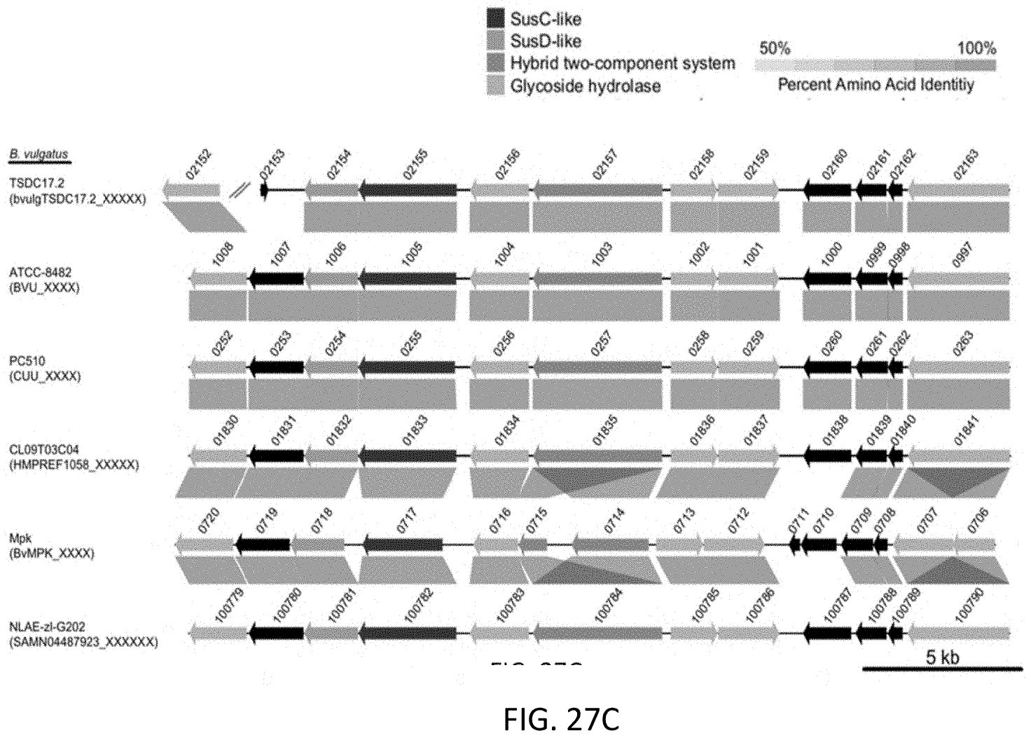

Genes in PULs were ranked by the magnitude of pea-fiber-dependent increases in the abundances their protein products and decreases in strain fitness when they were disrupted by a Tn insertion. The results revealed genes in three PULs (PUL7 in B. thetaiotaomicron, PUL5 in B. cellulosilyticus, and PUL27 in B. vulgatus; FIG. 2F, FIG. 2H, and FIG. 2I) that were affected by pea fiber. These three PULs are homologous as judged by a BLASTp comparison of their encoded proteins against the genomes of other community members (FIG. 2J). Genes in a highly-conserved arabinose utilization operon, present within the B. thetaiotaomicron and B. cellulosilyticus PULs, but at a site distant from PUL27 in B. vulgatus, had the greatest effect on B. vulgatus fitness of any genes represented in the mutant library (FIG. 2I; see also Table S6A of Patnode et al., Cell, 2019, 179(1): 59-73). We subsequently compared the genomes of five strains of B. thetaiotaomicron, and found that PUL7 was highly conserved with the exception of a single gene of unknown function (BT_0352) that was present in two of the strains (FIG. 27). PUL27 in B. vulgatus was also well conserved across 6 strains with the exception of some variability in the gene lengths of the hybrid two-component system and SusC-like transporter.

The increased fitness cost of mutations in B. ovatus RGI-processing PUL97, but not the B. thetaiotaomicron RGI-processing PUL75, indicated that these species utilize different carbohydrates in the pea fiber-supplemented diet (RGI and arabinan, respectively; FIG. 2G; see also Table S6A of Patnode et al., Cell, 2019, 179(1): 59-73). In contrast, the overlapping reliance on arabinan degradation pathways in B. thetaiotaomicron, B. vulgatus, and B. cellulosilyticus raised the possibility that these species were engaged in competition with one another for arabinan in pea fiber.

A parallel analysis of mice monotonously fed citrus pectin revealed that five genes encoded by galacturonan-processing PUL83 in B. cellulosilyticus were among the most abundantly expressed and most important for fitness compared to the base diet condition (FIG. 7H). B. vulgatus did not expand with citrus pectin supplementation (FIG. 7E), nevertheless, it contained galacturonan-processing PULs (PUL5/6, PUL31, and PUL42/43) with genes involved in hexuronate metabolism whose protein products increased in abundance and, when mutated, conveyed decreased fitness when exposed to this fiber preparation (FIG. 7I). Consistent with increased reliance on citrus pectin, the abundance of B. vulgatus proteins involved in starch utilization (PUL38) was decreased in the presence of this fiber.

Together, our proteomic and INSeq datasets revealed the microbial genes required during fiber-driven expansion, highlighted the polysaccharides that contributed to the fitness effects of these fibers and provided evidence for functional overlap in the nutrient harvesting strategies of B. cellulosilyticus and B. vulgatus, in two distinct fiber conditions. The dominance of B. cellulosilyticus in diverse diet contexts led us to ask whether (and how) this species directly competes with other community members for polysaccharides.

### Example 4—Interspecies Competition Controls the Outcomes of Fiber-Based Microbiota Manipulation

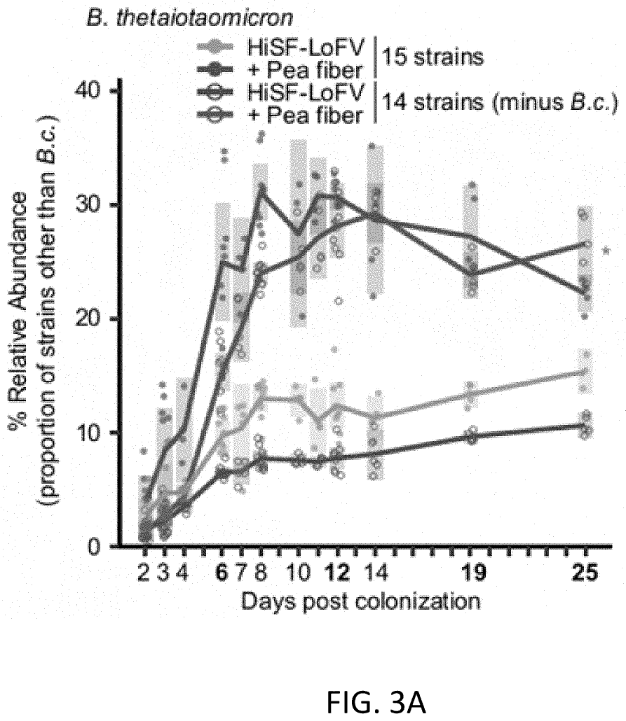

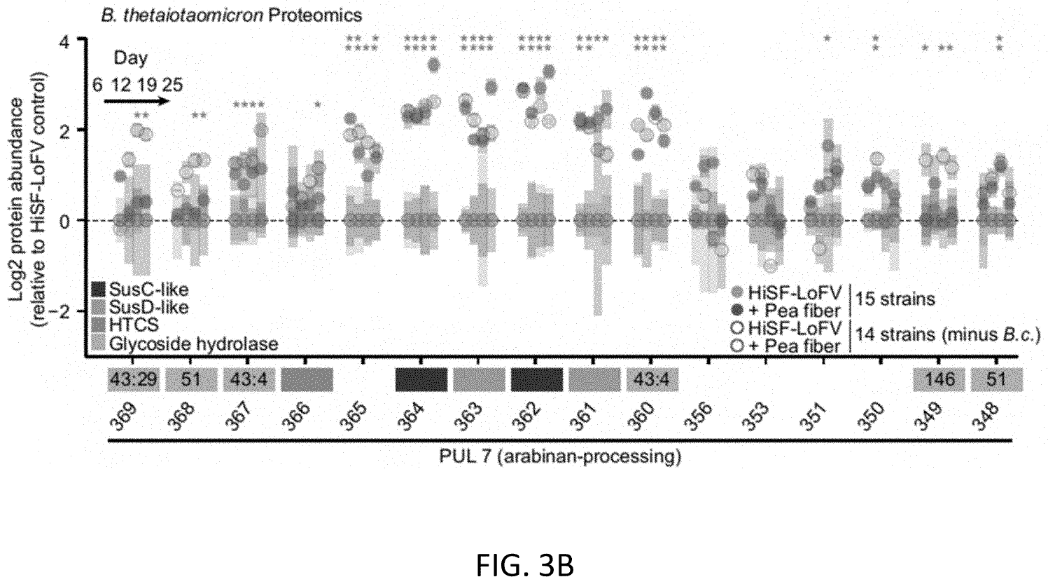

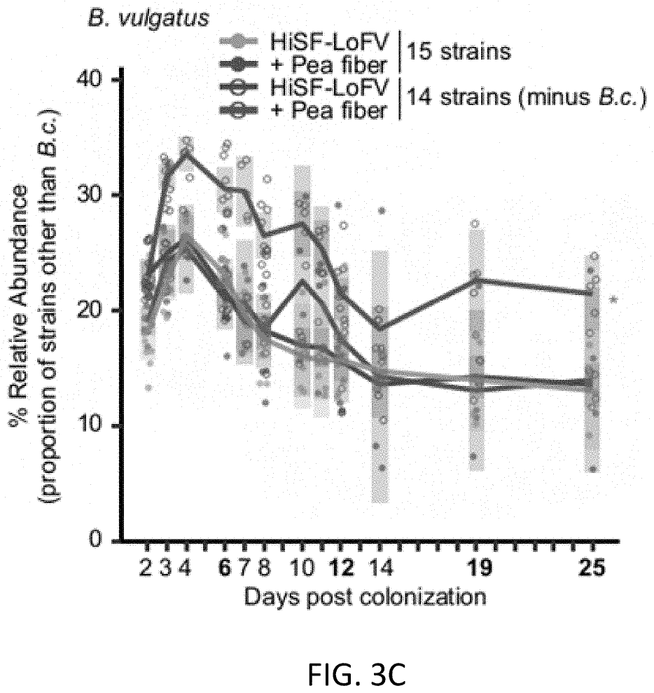

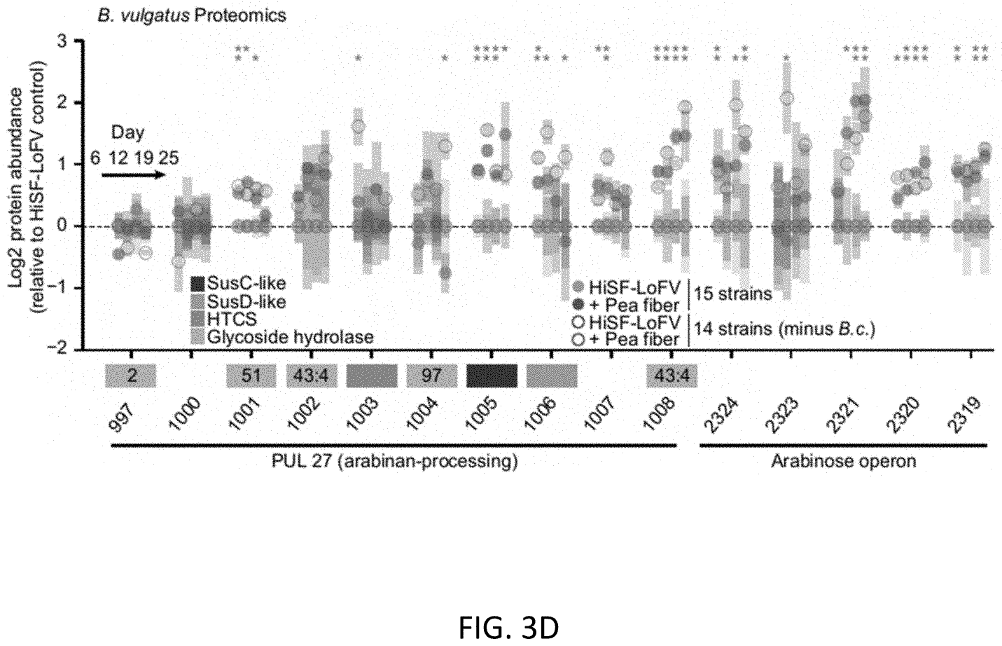

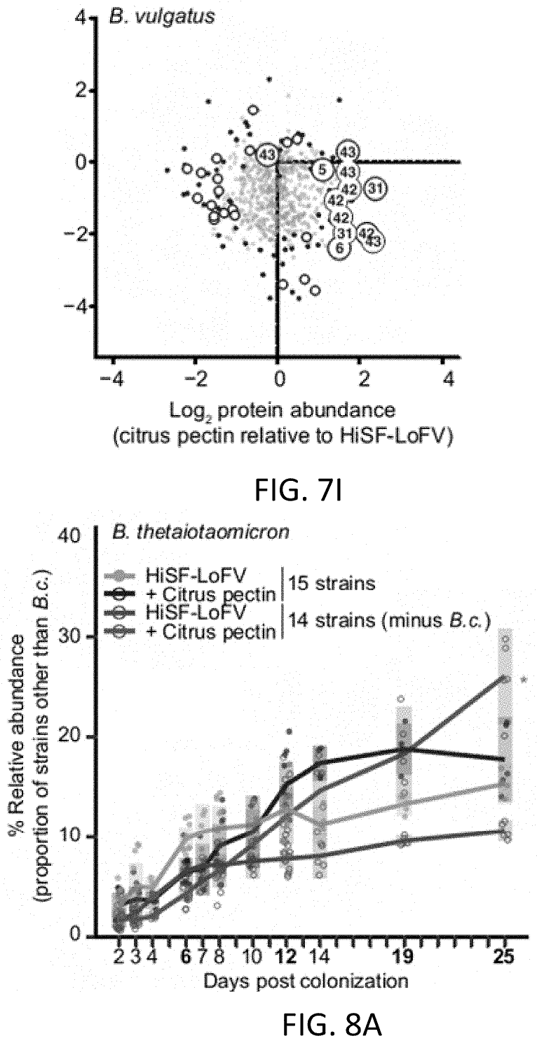

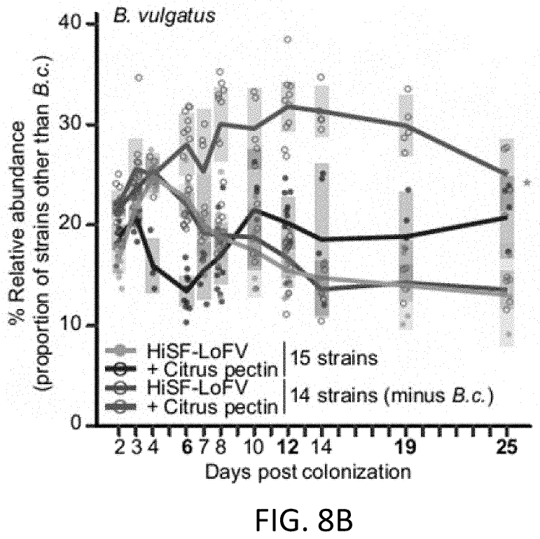

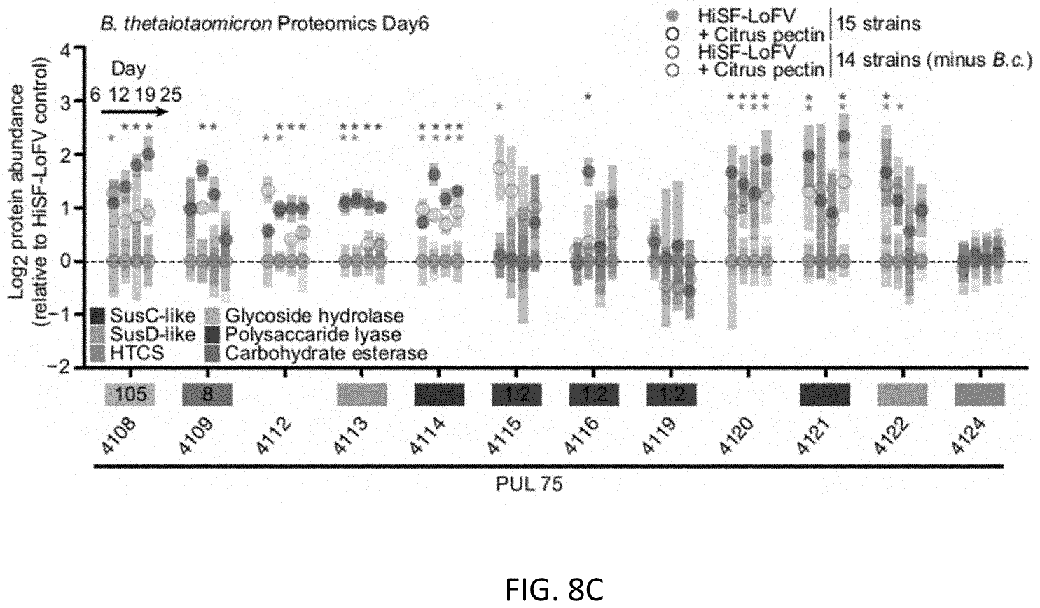

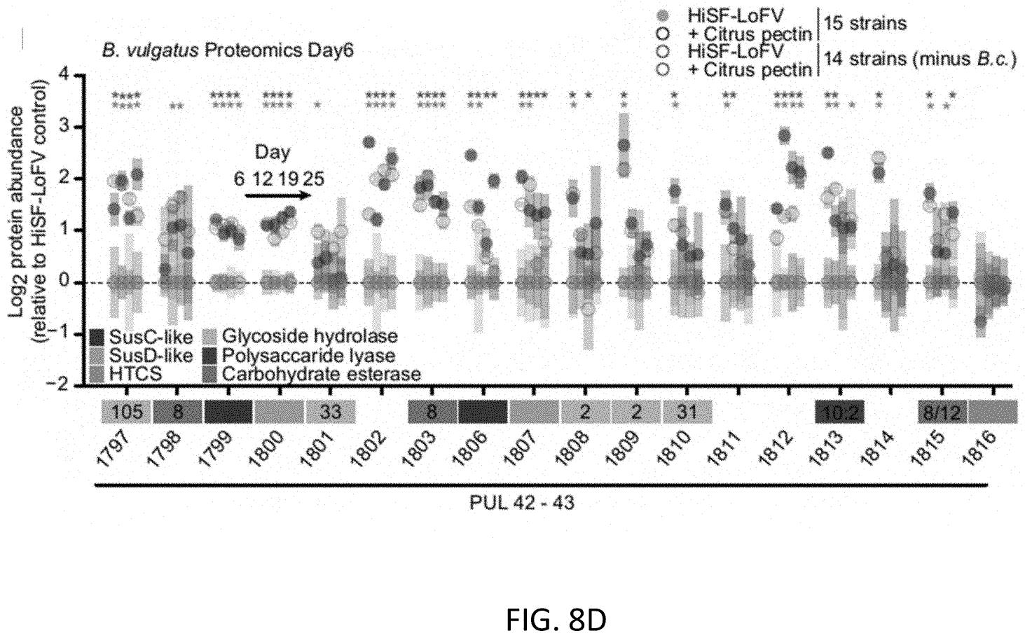

We performed a direct test for interactions between B. cellulosilyticus and other species by comparing the defined 15-member community, to the derivative 14-member community lacking B. cellulosilyticus. Using an experimental design that mimicked the monotonous feeding study described above, groups of germ-free mice were colonized with these two communities and fed the HiSF-LoFV diet with or without 10% (w/w) pea fiber or citrus pectin (see Tables S4B-S4C of Patnode et al., Cell, 2019, 179(1): 59-73). COPRO-Seq analysis was used to determine the abundance of each strain as a proportion of all strains other than B. cellulosilyticus, thereby controlling for the compositional effect of removing this species. Defined this way, the abundance of B. thetaiotaomicron did not increase upon omission of B. cellulosilyticus in the presence of pea fiber, suggesting minimal competition between these two species for arabinan (FIG. 3A; see also Tables S4B, C of Patnode et al., Cell, 2019, 179(1): 59-73). Proteomic analysis of fecal samples collected on experimental days 6, 12, 19, and 25 demonstrated that the proteins in B. thetaiotaomicron PUL7 whose abundances were increased by pea fiber in the complete community context, were not further increased in the absence of B. cellulosilyticus (FIG. 3B). B. vulgatus was the only species that expanded with pea fiber administration in the absence of B. cellulosilyticus (P<0.05, ANOVA, FDR corrected; FIG. 3C; see also Tables S4B, D of Patnode et al., Cell, 2019, 179(1): 59-73). Proteomic analysis of serially collected fecal samples disclosed that the abundances of proteins encoded by B. vulgatus PUL27, as well as its arabinose operon, were persistently increased during exposure to pea fiber, regardless of whether B. cellulosilyticus was included in the community (FIG. 3D). Citrus pectin provided a second example of fiber-driven expansion of B. vulgatus in the absence of B. cellulosilyticus (FIG. 8B; see also Tables S4B, D of Patnode et al., Cell, 2019, 179(1): 59-73). Expression of proteins encoded by B. vulgatus' galacturonan-processing PULs 5, 6, 31, 42, and 43, were also induced by citrus pectin, irrespective of B. cellulosilyticus (FIG. 8C and FIG. 8D). Odoribacter splanchnicus expanded in the absence of B. cellulosilyticus; this effect was repressed by both pea fiber and citrus pectin administration.

These results demonstrate negative interactions between B. vulgatus and B. cellulosilyticus and suggest that the suppression of B. vulgatus when B. cellulosilyticus is present occurs due to the persistent competition between these organisms for arabinan in pea fiber and homogalacturonan in citrus pectin.

### Example 5—Artificial Food Particles as Biosensors of Community Glycan Degradative Activities

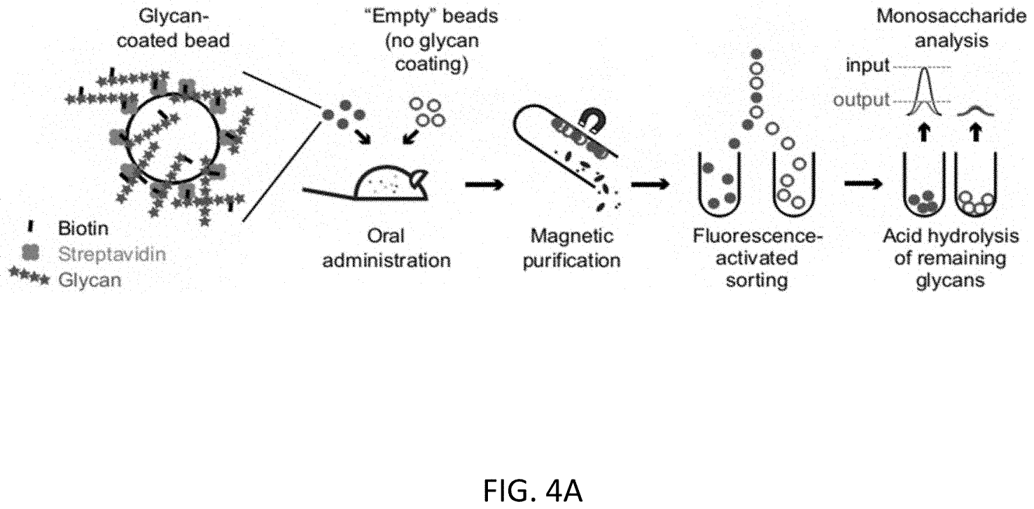

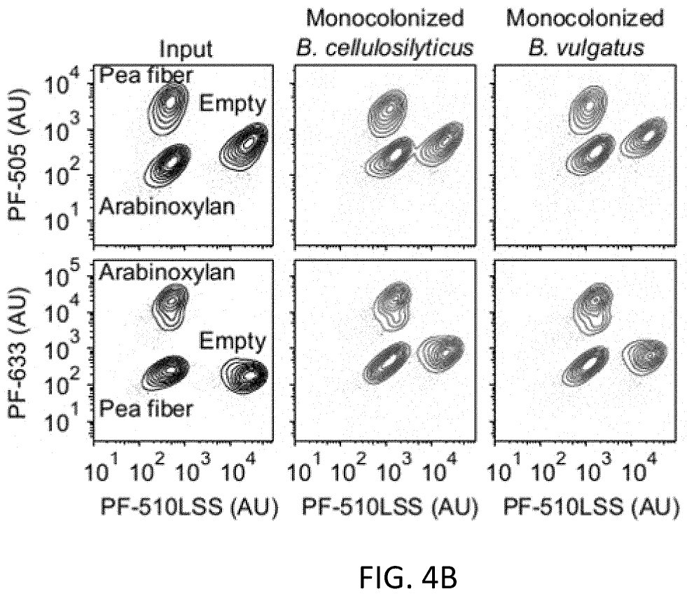

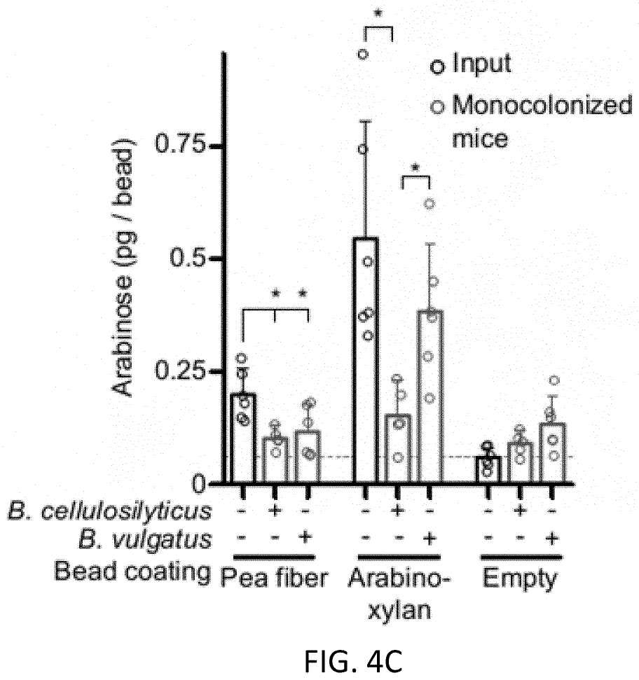

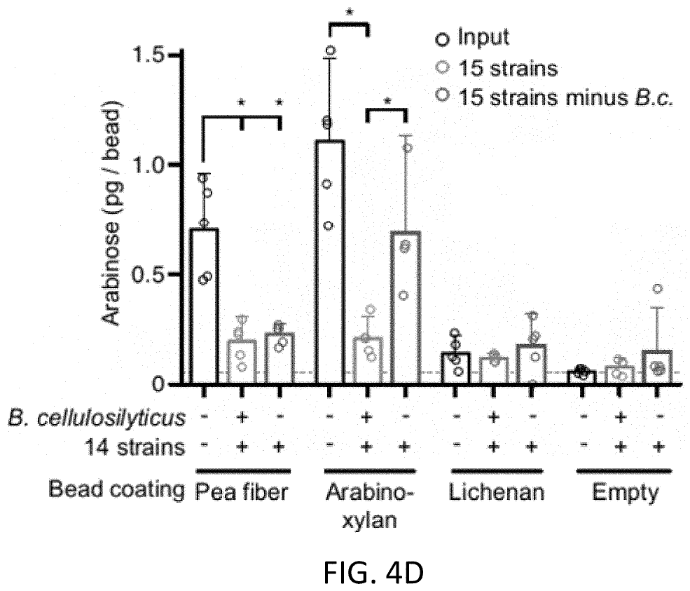

To directly test the capacity of competing Bacteroides to process the same nutrient substrate in vivo, a bead-based glycan degradation assay was developed (FIG. 4A). Two polysaccharides of interest were selected: (i) a soluble, starch-depleted fraction of pea fiber polysaccharides composed predominantly of arabinose (83% of monosaccharides) with little xylose (4%), and (ii) wheat arabinoxylan (38% arabinose/62% xylose). The latter was used as a control given its established ability to support growth (in vitro) of B. cellulosilyticus (McNulty et al., 2013) but not B. vulgatus (Tauzin et al., 2016). These polysaccharides were biotinylated and each product was attached to a distinct population of microscopic (20 μm diameter) streptavidin-coated paramagnetic glass beads, generating carbohydrate-coated artificial ‘food particles’ that could be recovered from mouse intestinal contents using a magnetic field. Each population of beads was also labeled with a distinct biotinylated fluorophore so that several types of polysaccharide-beads could be pooled, administered at the same time to the same mouse, recovered from the gut lumen or feces and then sorted into their original groups using a flow cytometer (FIG. 4B). ‘Empty’ beads that had not been incubated with polysaccharides, but were labeled with a unique biotinylated fluorophore, served as negative controls. The sorted beads were subjected to acid hydrolysis and the hydrolysis products were assayed by gas chromatography-mass spectrometry (GC-MS) to quantify the levels of bead-bound carbohydrate present before and after transit through the mouse gut. Alternative methods to quantify the levels of bead-bound carbohydrate present before and after administration can also be used.

Germ-free mice were colonized with either B. cellulosilyticus or B. vulgatus alone and fed a HiSF-LoFV diet supplemented with 10% (w/w) pea fiber. Seven days after colonization, all mice were gavaged with an equal mixture of the three bead types (5×10⁶ of each type/animal, n=5-6 animals). Mice were euthanized 4 h later, beads were recovered from their cecum and colon, and the mass of monosaccharides on the different purified bead types was quantified. The fluorescent signal present on all bead types persisted after intestinal transit, confirming that the biotin-streptavidin interactions were stable under these conditions (FIG. 4B). Pea fiber-beads recovered from both groups of mice had significantly reduced arabinose [26.1±3.4% (mean±SD) and 29.1±0.7% of levels in input beads, respectively]. In contrast, levels of arabinose were only significantly decreased on arabinoxylan-coated beads recovered from mice colonized with B. cellulosilyticus (FIG. 4C; Table 9).

A follow-up experiment of identical design was performed except that animals fed HiSF-LoFV supplemented with pea fiber were gavaged 12 days rather than seven days after colonization with a collection of four rather than three types of beads. These beads were either empty (no glycan bound) or coated with (i) the soluble, starch-depleted fraction of pea fiber, or wheat arabinoxylan, or lichenan from Icelandic moss, a control glycan low in arabinose (81% glucose/8% mannose/6% galactose/2% arabinose). Beads were recovered, purified by flow cytometry and analyzed using GC-MS. The degradation of bead-bound pea fiber and arabinoxylan was similar to that observed on day 7.

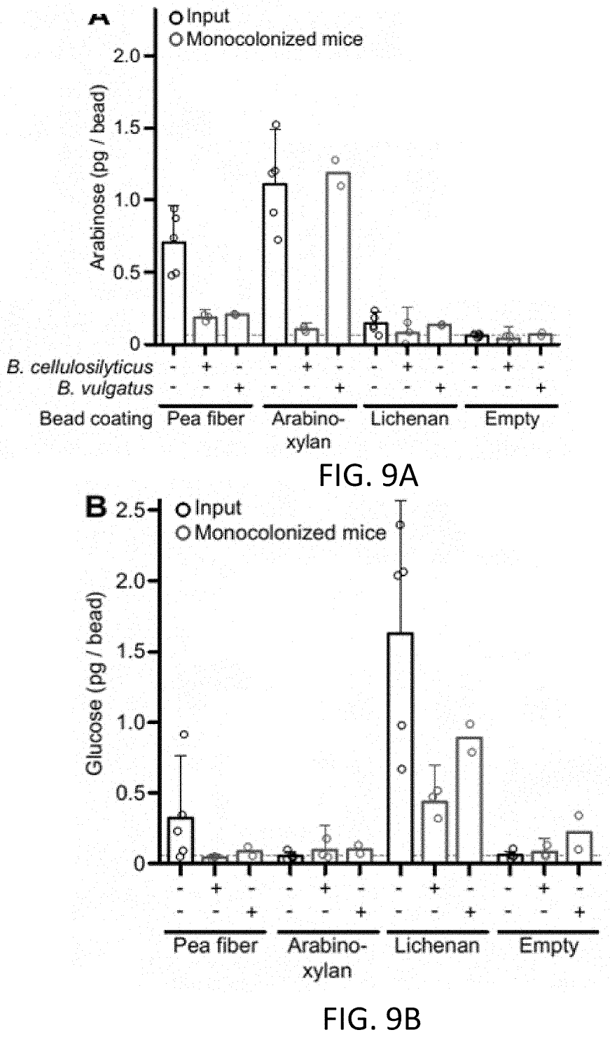

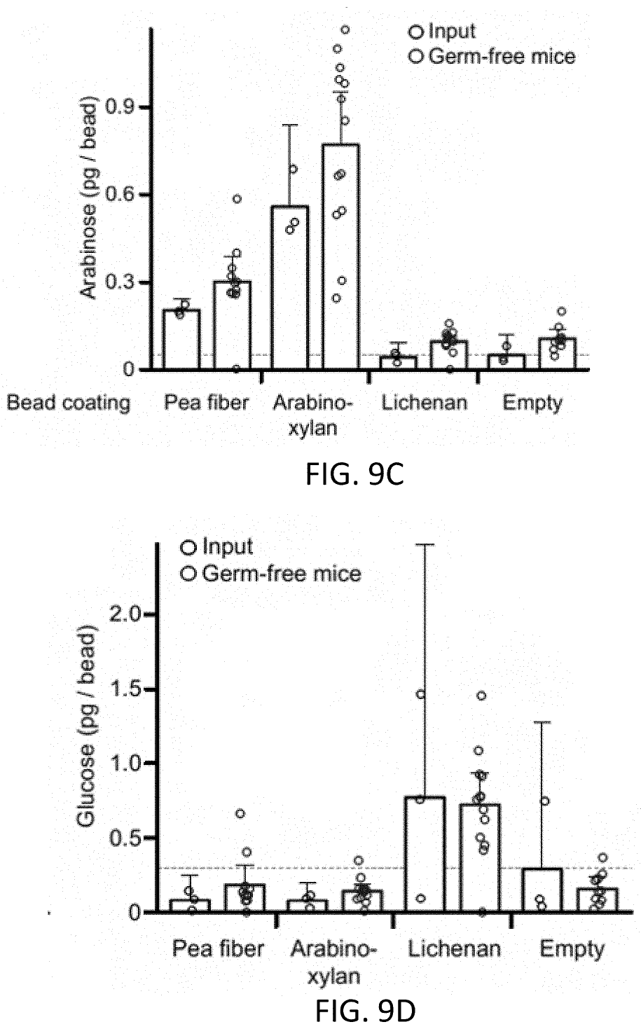

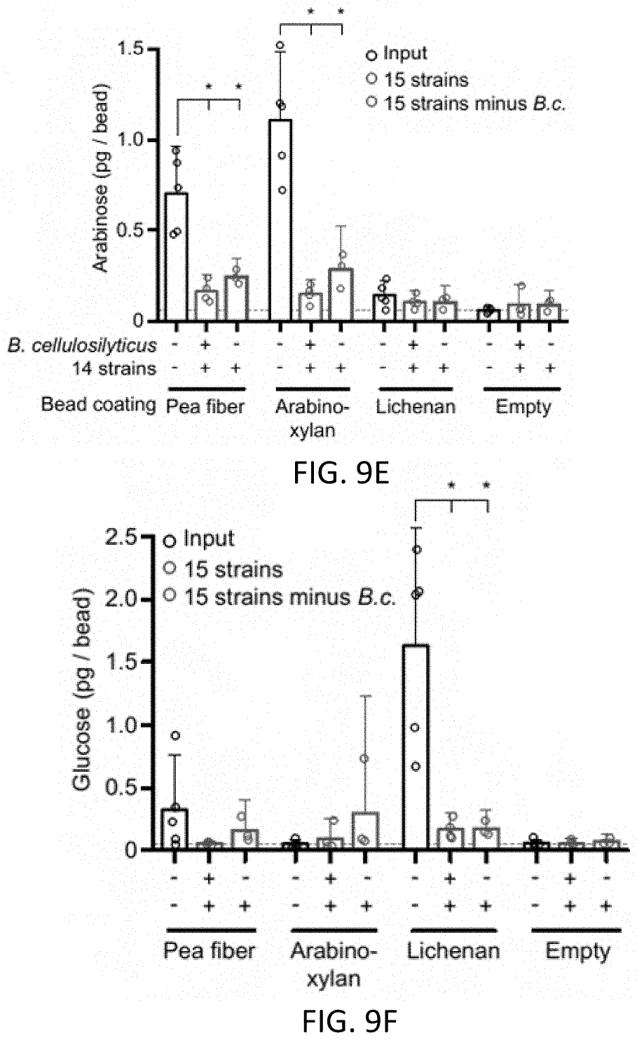

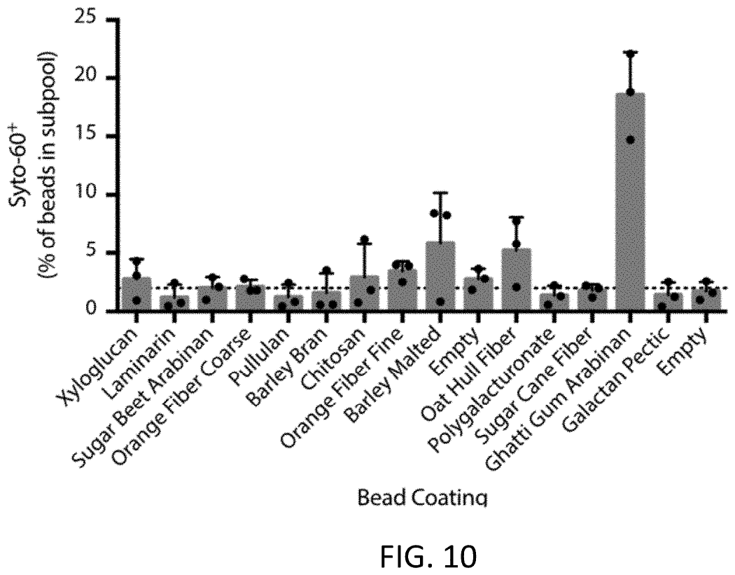

To control for microbe-independent polysaccharide degradation, germ-free mice were given a gavage of arabinoxylan-coated, pea-fiber coated, lichenan-coated, and empty beads (n=13 animals). We collected all fecal samples produced during an 8 h period (from 4 to 12 hours after gavage). Assays of the arabinoxylan-, pea fiber-, and lichenan-coated beads purified from fecal samples obtained from each germ-free animal revealed no significant degradation of these polysaccharides after passage through their intestines (FIG. 9C and FIG. 9D; Tables 8 and 9). Together, these results provide a direct, in vivo demonstration of the overlapping capacities of competing Bacteroides species to degrade arabinan present in pea fiber.

Given the observation that several species can metabolize pea fiber arabinan in vivo, whether the absence of B. cellulosilyticus would compromise the efficiency with which the community carried out this function was assessed. Mice consuming the unsupplemented HiSF-LoFV diet were given pea fiber-coated, arabinoxylan-coated, lichenan-coated, and empty beads 12 days after colonization with (i) the 15-member consortium or (ii) the derivative 14-member community lacking B. cellulosilyticus. Analysis of beads recovered from the cecal and colonic contents of these mice disclosed that the level of pea fiber degradation was not affected by the absence of B. cellulosilyticus (FIG. 4D). In a separate group of mice fed a pea-fiber supplemented HiSF-LoFV diet, degradation of bead-bound pea-fiber was also the same regardless of the presence of B. cellulosilyticus (FIG. 9E and FIG. 9F).

Thus, these artificial food particles provide a way to conduct in vivo assessments of dietary nutrient degradation by microbes as a function of community composition. Consistent with our detection of multiple species exploiting pea fiber arabinan as a nutrient source (FIG. 2 and FIG. 3), this community can compensate for the loss of B. cellulosilyticus-mediated arabinan degradation. In contrast, the breakdown of dietary arabinoxylan represents a non-redundant function provided by B. cellulosilyticus.

TABLE 8

| | | | | B. vulgatus | | B. cellulosilyticus | |
|---|---|---|---|---|---|---|---|
| | | Input | | HiSF-LoFV | | HiSF-LoFV | |
| | | Mean | | Mean | | Mean | |
| | Monosaccharide | (pg/bead) | sd | (pg/bead) | sd | (pg/bead) | sd |
| Xy | Arabinoxylan beads | 0.17 | 0.12 | 0.11 | 0.12 | 0.05 | 0.05 |
| | Pea Fiber beads | 0.06 | 0.07 | 0.01 | 0.01 | 0.01 | 0.02 |
| | Uncoated beads | 0.01 | 0.01 | 0.01 | 0.01 | 0.02 | 0.03 |
| Ara | Arabinoxylan beads | 0.54 | 0.25 | 0.38 | 0.15 | 0.15 | 0.07 |
| | Pea Fiber beads | 0.2 | 0.06 | 0.12 | 0.06 | 0.1 | 0.02 |
| | Uncoated beads | 0.06 | 0.02 | 0.13 | 0.06 | 0.09 | 0.02 |
| Man | Arabinoxylan beads | 0.02 | 0.02 | 0.04 | 0.03 | 0.05 | 0.03 |
| | Pea Fiber beads | 0.04 | 0.02 | 0.06 | 0.04 | 0.06 | 0.04 |
| | Uncoated beads | 0.06 | 0.03 | 0.14 | 0.07 | 0.1 | 0.06 |
| Gal | Arabinoxylan beads | 0.02 | 0.01 | 0.06 | 0.05 | 0.02 | 0.01 |
| | Pea Fiber beads | 0.05 | 0.03 | 0.07 | 0.05 | 0.05 | 0.02 |
| | Uncoated beads | 0 | 0.01 | 0.05 | 0.03 | 0.04 | 0.2 |
| Glc | Arabinoxylan beads | 0.02 | 0.04 | 0 | 0.01 | 0.01 | 0.01 |
| | Pea Fiber beads | 0.01 | 0.02 | 0 | 0.01 | 0 | 0 |
| | Uncoated beads | 0.01 | 0.01 | 0 | 0.01 | 0.01 | 0.01 |

Abbreviations:
xylose (Xyl),
arabinose (Ara),
mannose (Man),
galactose (Gal),
glucose (Glc)

TABLE 9A

| | | Mean (sd) pg/bead | | | | | | | | | |
|---|---|---|---|---|---|---|---|---|---|---|---|
| | BEAD TYPE | Input | | A | | B | | C | | D | |
| Xyl | Arabinoxylan | 2.1 | (0.55) | 0.44 | (0.21) | 0.85 | (0.24) | 0.23 | (0.18) | 0.36 | (0.11) |
| | Pea Fiber | 0.2 | (0.29) | 0.09 | (0.07) | 0.06 | (0.03) | 0.03 | (0.02) | 0.08 | (0.04) |
| | Lichenan | 0.06 | (0.02) | 0.06 | (0.05) | 0.11 | (0.09) | 0.04 | (0.02) | 0.08 | (0.04) |
| | Uncoated | 0.07 | (0.02) | 0.21 | (0.37) | 0.07 | (0.07) | 0.1 | (0.14) | 0.43 | (0.52) |
| Ara | Arabinoxylan | 1.11 | (0.31) | 0.21 | (0.08) | 0.69 | (0.28) | 0.15 | (0.05) | 0.28 | (0.1) |
| | Pea Fiber | 0.7 | (0.21) | 0.2 | (0.09) | 0.23 | (0.04) | 0.16 | (0.06) | 0.24 | (0.04) |
| | Lichenan | 0.14 | (0.07) | 0.12 | (0.02) | 0.17 | (0.12) | 0.11 | (0.04) | 0.1 | (0.04) |
| | Uncoated | 0.06 | (0.01) | 0.08 | (0.03) | 0.15 | (0.16) | 0.09 | (0.07) | 0.09 | (0.03) |
| Man | Arabinoxylan | 0 | (0.01) | 0.02 | (0.01) | 0.02 | (0.01) | 0.02 | (0.01) | 0.02 | (0.01) |
| | Pea Fiber | 0.05 | (0.04) | 0.04 | (0.01) | 0.09 | (0.04) | 0.02 | (0.01) | 0.03 | (0.01) |
| | Lichenan | 0.12 | (0.05) | 0.14 | (0.06) | 0.17 | (0.13) | 0.09 | (0.03) | 0.07 | (0.03) |
| | Uncoated | 0 | (0.01) | 0.04 | (0.01) | 0.04 | (0.02) | 0.02 | (0.01) | 0.03 | (0.01) |
| Gal | Arabinoxylan | 0.02 | (0.01) | 0.09 | (0.02) | 0.11 | (0.06) | 0.09 | (0.02) | 0.07 | (0.01) |
| | Pea Fiber | 0.22 | (0.29) | 0.21 | (0.04) | 0.35 | (0.14) | 0.13 | (0.04) | 0.18 | (0.05) |
| | Lichenan | 0.23 | (0.1) | 0.2 | (0.14) | 0.44 | (0.29) | 0.21 | (0.1) | 0.29 | (0.09) |
| | Uncoated | 0.02 | (0.01) | 0.21 | (0.08) | 0.27 | (0.13) | 0.12 | (0.03) | 0.11 | (0.04) |
| Glc | Arabinoxylan | 9.15 | (7.95) | 11.53 | (7.18) | 14.21 | (11.64) | 8 | (3.43) | 23.27 | (17.41) |
| | Pea Fiber | 38.2 | (33.13) | 12.81 | (3.85) | 22.32 | (8.36) | 6.91 | (1.14) | 20.48 | (10.25) |
| | Lichenan | 193.16 | (71.35) | 23.41 | (4.76) | 37.74 | (26.01) | 24.89 | (8.33) | 19.62 | (7.81) |
| | Uncoated | 8.41 | (4.65) | 11.3 | (5.51) | 14.49 | (10.38) | 7.12 | (2.65) | 11.68 | (1.3) |

A = 15-member, HiSF-LoFV;
B = 14-member (No B.c.), HiSF-LoFV;
C = 15-member HiSF-LoFV + Pea Fiber;
D = 14-member (No B.c), HiSF-LoFV + Pea Fiber
Abbreviations:
xylose (Xyl),
arabinose (Ara),
mannose (Man),
galactose (Gal),
glucose (Glc)

TABLE 9B

Mean (sd) pg/bead

| | BEAD TYPE | E | F |
|---|---|---|---|
| Xyl | Arabinoxylan | 0.22 0.18 | 1.46 0.24 |
| | Pea Fiber | 0.14 0.15 | 0.06 0.01 |
| | Lichenan | 0.04 0.02 | 0.14 0.09 |
| | Uncoated | 0.16 0.04 | 0.1 0.04 |
| Ara | Arabinoxylan | 0.1 0.02 | 1.19 0.12 |
| | Pea Fiber | 0.18 0.02 | 0.2 0.01 |
| | Lichenan | 0.08 0.08 | 0.14 0.01 |
| | Uncoated | 0.04 0.03 | 0.07 0.01 |
| Man | Arabinoxylan | 0.03 0.03 | 0.01 0.01 |
| | Pea Fiber | 0.03 0.02 | 0.02 0.01 |
| | Lichenan | 0.1 0.04 | 0.1 0.02 |
| | Uncoated | 0.06 0.03 | 0.02 0.01 |
| Gal | Arabinoxylan | 0.15 0.14 | 0.1 0.02 |
| | Pea Fiber | 0.25 0.12 | 0.22 0.02 |
| | Lichenan | 0.36 0.15 | 0.46 0.18 |
| | Uncoated | 0.27 0.13 | 0.2 0.06 |
| Glc | Arabinoxylan | 7.13 5.46 | 8.39 0.81 |
| | Pea Fiber | 8.35 3.84 | 8.34 2.2 |
| | Lichenan | 23.52 3.84 | 69.59 15.85 |
| | Uncoated | 14.47 2.24 | 17.82 5.76 |

E = B. cellulosilyticus, HiSF-LoFV+ Pea Fiber;
F = B. vulgatus, HiSF-LoFV+ Pea Fiber
Abbreviations:
xylose (Xyl),
arabinose (Ara),
mannose (Man),
galactose (Gal),
glucose (Glc)

TABLE 10

| | | | | Germ-free | |
|---|---|---|---|---|---|
| | | Input | | HiSF-LoFV | |
| | | Mean | | Mean | |
| | Monosaccharide | (pg/bead) | sd | (pg/bead) | sd |
| Xyl | Arabinoxylan beads | 1.18 | 0.16 | 1.35 | 0.41 |
| | Pea Fiber beads | 0.11 | 0.08 | 0.22 | 0.26 |
| | Lichenan beads | 0.16 | 0.22 | 0.14 | 0.08 |
| | Uncoated beads | 0.11 | 0.03 | 0.18 | 0.06 |
| Ara | Arabinoxylan beads | 0.56 | 0.11 | 0.77 | 0.3 |
| | Pea Fiber beads | 0.2 | 0.02 | 0.3 | 0.14 |
| | Lichenan beads | 0.04 | 0.02 | 0.1 | 0.04 |
| | Uncoated beads | 0.05 | 0.03 | 0.11 | 0.04 |
| Man | Arabinoxylan beads | 0.01 | 0.01 | 0.05 | 0.05 |
| | Pea Fiber beads | 0.03 | 0.02 | 0.05 | 0.03 |
| | Lichenan beads | 0.12 | 0.07 | 0.23 | 0.14 |
| | Uncoated beads | 0.02 | 0.01 | 0.03 | 0.01 |
| Gal | Arabinoxylan beads | 0.03 | 0.01 | 0.2 | 0.07 |
| | Pea Fiber beads | 0.06 | 0.03 | 0.37 | 0.14 |
| | Lichenan beads | 0.2 | 0.1 | 0.59 | 0.23 |
| | Uncoated beads | 0.07 | 0.08 | 0.26 | 0.11 |
| Glc | Arabinoxylan beads | 13.72 | 3.3 | 13.78 | 5.2 |
| | Pea Fiber beads | 7.27 | 4.32 | 25.47 | 22.97 |
| | Lichenan beads | 79.39 | 40.38 | 92.09 | 57.16 |
| | Uncoated beads | 22.58 | 25 | 22.24 | 19.15 |

Abbreviations:
xylose (Xyl),
arabinose (Ara),
mannose (Man),
galactose (Gal),
glucose (Glc)

TABLE 11

| | | Mean (sd) pg/bead | | | | | | | | | |
|---|---|---|---|---|---|---|---|---|---|---|---|
| | BEAD TYPE | Input | | A | | B | | C | | D | |
| Xyl | Arabinoxylan | 0.15 | (0.09) | 0.1 | (0.02) | 0.13 | (0.07) | 0.22 | (0.33) | 0.11 | (0.07) |
| | Mannan | 0.38 | (0.16) | 0.31 | (0.07) | 0.23 | (0.17) | 0.26 | (0.12) | 0.32 | (0.25) |
| | Uncoated | 0.05 | (0.05) | 0.08 | (0.04) | 0.44 | (0.66) | 0.09 | (0.05) | 0.08 | (0.01) |
| | Arabinoxylan (spike- | 0.1 | (0.03) | 0.17 | (0.21) | 0.43 | (0.75) | 0.17 | (0.14) | 0.09 | (0.05) |
| | in control) | | | | | | | | | | |
| Ara | Arabinoxylan | 9.02 | (2.84) | 3.07 | (0.68) | 3.73 | (1.57) | 4.41 | (4.08) | 8.45 | (3.53) |
| | Mannan | 1.09 | (2.46) | 0.09 | (0.05) | 0.04 | (0.02) | 0.07 | (0.06) | 0.1 | (0.05) |
| | Uncoated | 0.29 | (0.42) | 0.1 | (0.04) | 0.06 | (0.03) | 0.1 | (0.06) | 0.24 | (0.1) |
| | Arabinoxylan (spike- | 7.44 | (3.09) | 8.61 | (4.43) | 8.02 | (2.38) | 10.52 | (1.95) | 8.67 | (6.21) |
| | in control) | | | | | | | | | | |
| Man | Arabinoxylan | 7.15 | (2.24) | 2.07 | (0.49) | 3.61 | (1.86) | 2.83 | (2.48) | 6.43 | (2.87) |
| | Mannan | 1.18 | (1.93) | 0.45 | (0.39) | 0.27 | (0.16) | 0.55 | (0.18) | 0.34 | (0.19) |
| | Uncoated | 0.26 | (0.4) | 0.1 | (0.05) | 0.12 | (0.07) | 0.1 | (0.02) | 0.17 | (0.02) |
| | Arabinoxylan (spike- | 5.82 | (2.39) | 5.46 | (2.11) | 7.06 | (2.31) | 6.16 | (1.72) | 5.78 | (3.89) |
| | in control) | | | | | | | | | | |
| Gal | Arabinoxylan | 0.17 | (0.1) | 0.12 | (0.02) | 0.16 | (0.05) | 0.17 | (0.1) | 0.15 | (0.07) |
| | Mannan | 0.23 | (0.29) | 0.11 | (0.1) | 0.06 | (0.05) | 0.1 | (0.06) | 0.08 | (0.06) |
| | Uncoated | 0.02 | (0.01) | 0.06 | (0.02) | 0.06 | (0.03) | 0.08 | (0.02) | 0.07 | (0.02) |
| | Arabinoxylan (spike- | 0.14 | (0.04) | 0.17 | (0.09) | 0.14 | (0.02) | 0.8 | (0.05) | 0.12 | (0.08) |
| | in control) | | | | | | | | | | |
| Glc | Arabinoxylan | 0.06 | (0.03) | 0.03 | (0.03) | 0.05 | (0.02) | 0.06 | (0.09) | 0.05 | (0.04) |
| | Mannan | 1.08 | (0.49) | 1.09 | (0.53) | 0.85 | (0.83) | 0.87 | (0.6) | 1.15 | (0.8) |
| | Uncoated | 0.02 | (0.02) | 0.04 | (0.03) | 0.05 | (0.04) | 0.04 | (0.03) | 0.07 | (0.06) |
| | Arabinoxylan (spike- | 0.04 | (0.02) | 0.03 | (0.02) | 0.04 | (0.03) | 0.02 | (0.02) | 0.03 | (0.03) |
| | in control) | | | | | | | | | | |

A = 15-member, HiSF-LoFV;
B = 14-member (No B.c.), HiSF-LoFV;
C = 14-member (No B.o.), HiSFmBO;
D = 13-member (No B.c., No B.o.), HiSF-LoFVmBO
Abbreviations:
xylose (Xyl),
arabinose (Ara),
mannose (Man),
galactose (Gal),
glucose (Glc)

### Example 6—Acclimation to the Presence of a Potential Competitor Alleviates Resource Conflict

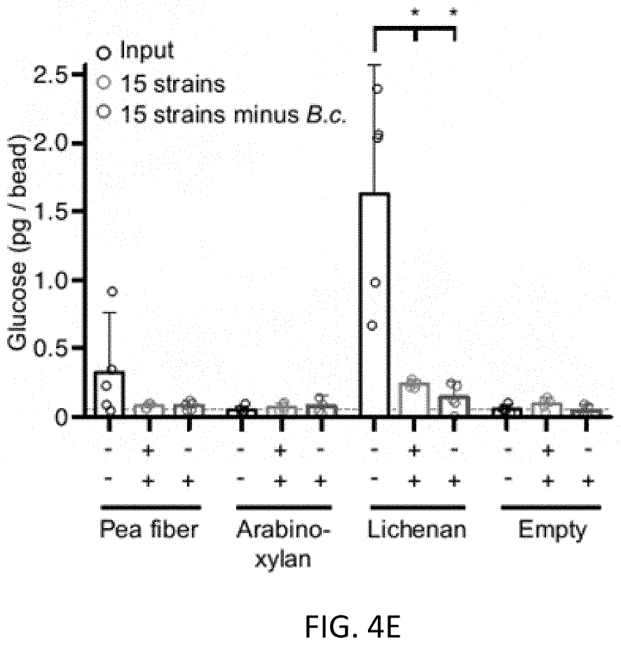

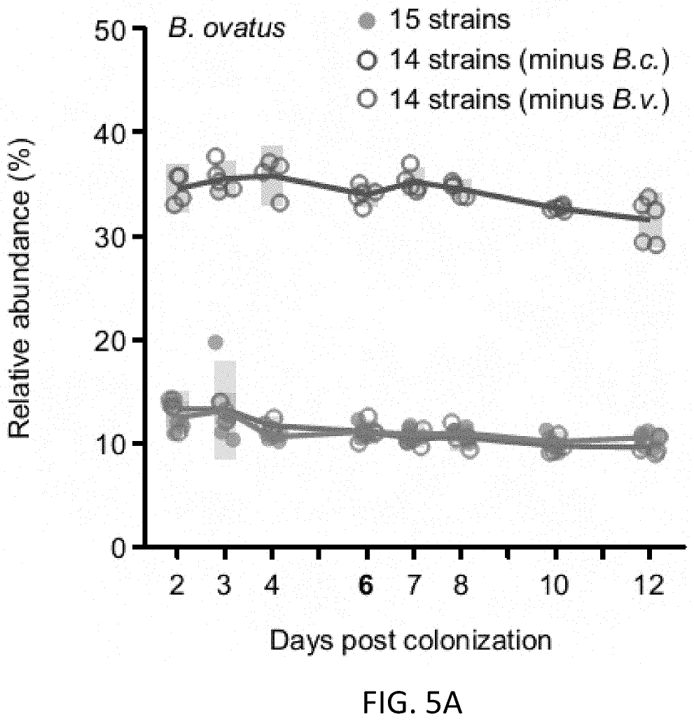

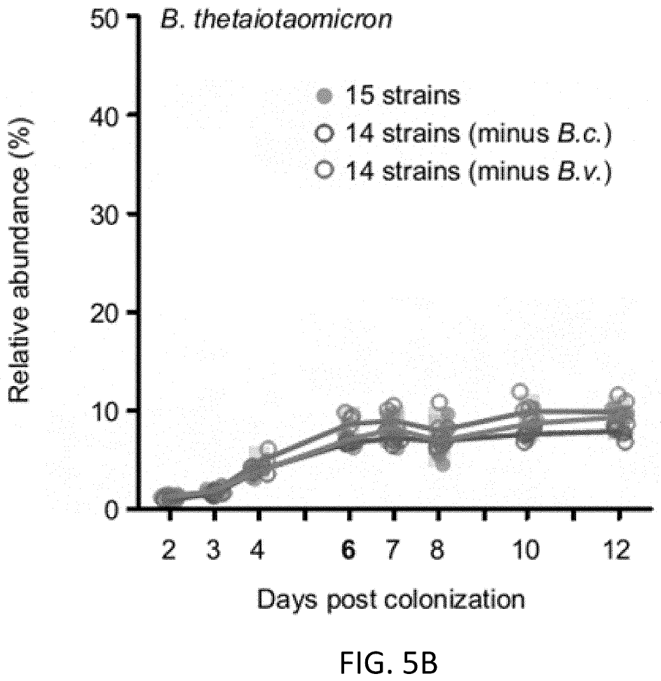

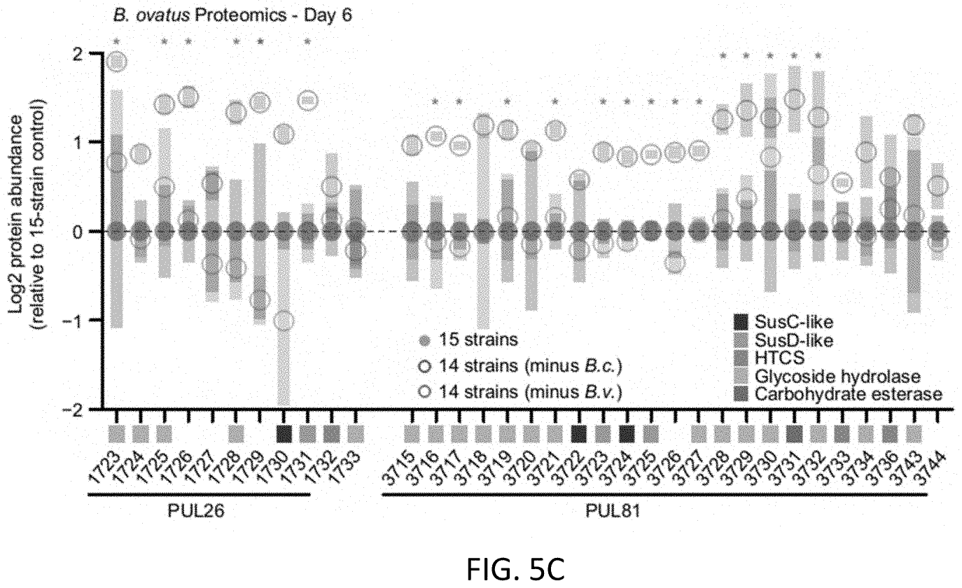

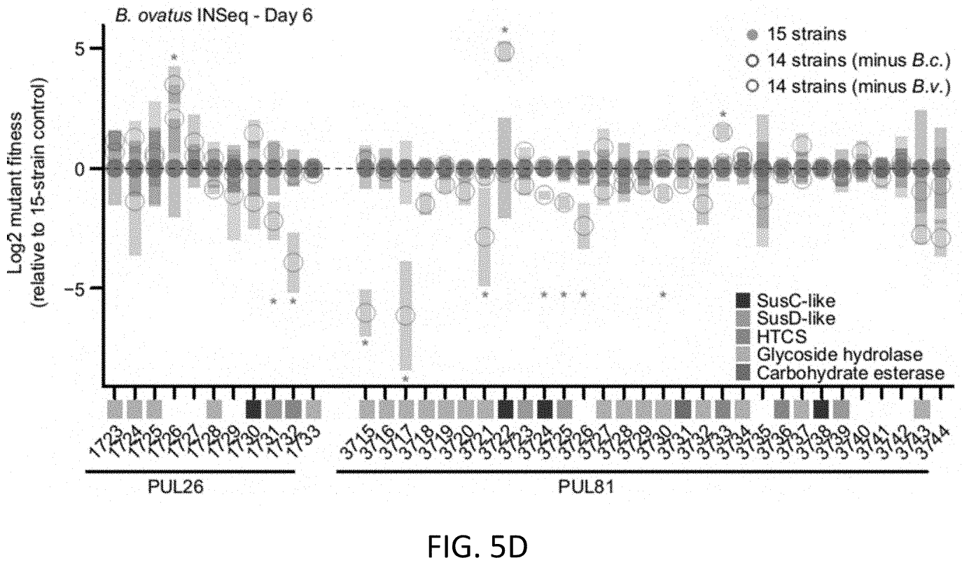

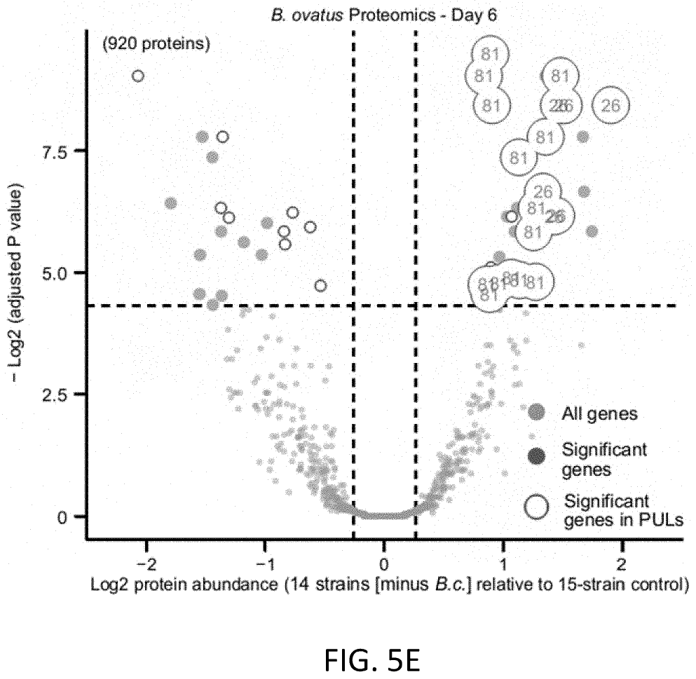

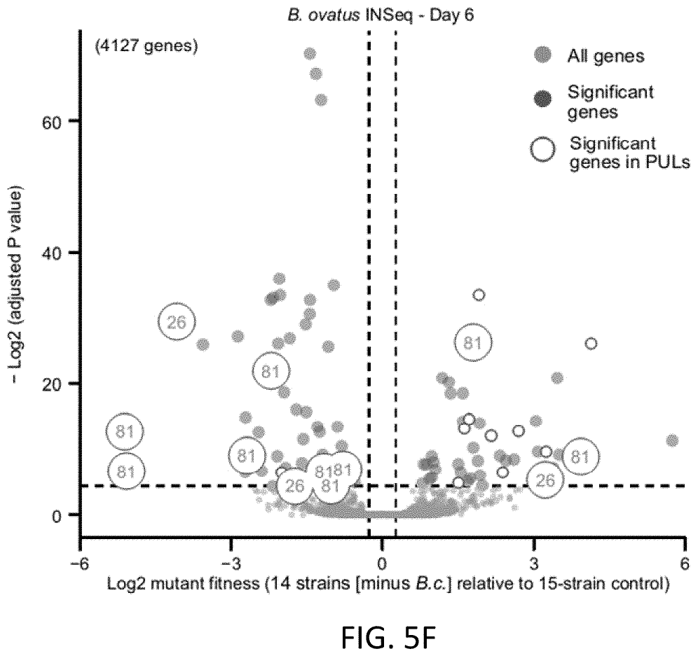

The in vivo bead-based glycan degradation assays revealed that in contrast to arabinan, the capacity of the community to process arabinoxylan was not rescued by other species in the absence of B. cellulosilyticus (FIG. 4D; Tables 8-11). This was unexpected, given that B. cellulosilyticus omission resulted in a significant increase in the relative abundance of B. ovatus (FIG. 5; see also Table S4 of Patnode et al., Cell, 2019, 179(1): 59-73), which encodes PULs capable of arabinoxylan breakdown (Martens et al., 2011; Rogowski et al., 2015). We examined whether these results could arise from a type of interspecies relationship between B. cellulosilyticus and B. ovatus distinct from that observed between B. cellulosilyticus and B. vulgatus.

As discussed above, the abundances of B. vulgatus proteins involved in pea fiber or citrus pectin degradation were unchanged upon removal of its competitor B. cellulosilyticus. In contrast, B. ovatus exhibited metabolic flexibility, with proteins encoded by two arabinoxylan-processing PULs (PUL26 and PUL81) predominating among those whose abundances were increased when B. cellulosilyticus was absent versus present (FIG. 5C and FIG.). This effect was apparent regardless of whether mice were fed the pea fiber-supplemented, citrus pectin-supplemented, or control unsupplemented HiSF-LoFV diets, consistent with the presence of arabinoxylan in the HiSF-LoFV diet. When we analyzed the contributions of genes to the fitness of B. ovatus (by calculating the changes in the abundance of Tn mutant strains from day 2 to day 6), those in these two arabinoxylan PULs were the most affected by omission of B. cellulosilyticus (FIG. 5D and FIG. 5F; see also Table S4B of Patnode et al., Cell, 2019, 179(1): 59-73). This result indicates that B. ovatus exhibits a marked decrease in its reliance on arabinoxylan in the full 15-member community context. Examining another group of mice that received a 14-member community lacking B. vulgatus revealed that its absence did not induce changes in B. ovatus at the level of its relative abundance, the abundances of proteins encoded by its PULs involved in arabinoxylan processing or by other PULs, or in the fitness cost associated with mutations in its arabinoxylan-processing PULs or in other PULs (FIG. 5A and FIG. 5C, and FIG. 5D; see also Tables S4D, A5F, and S6B of Patnode et al., Cell, 2019, 179(1): 59-73).

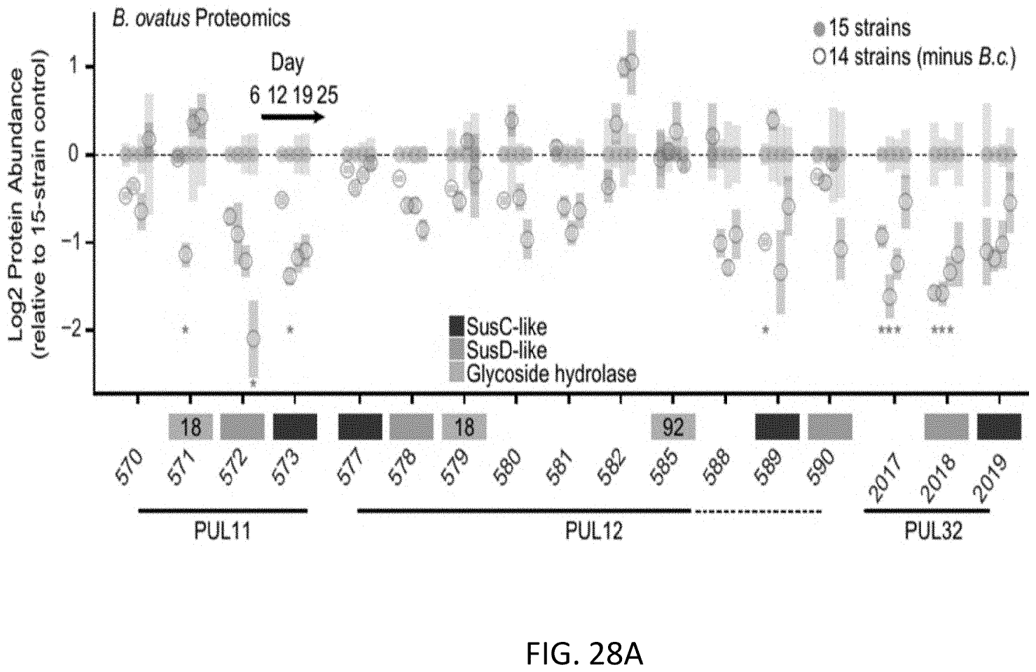

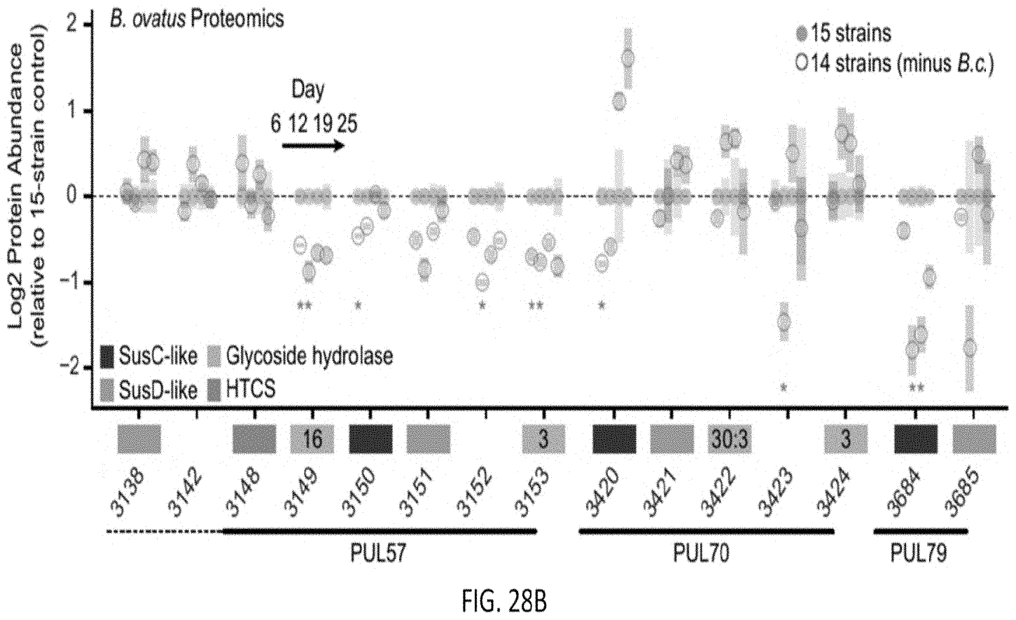

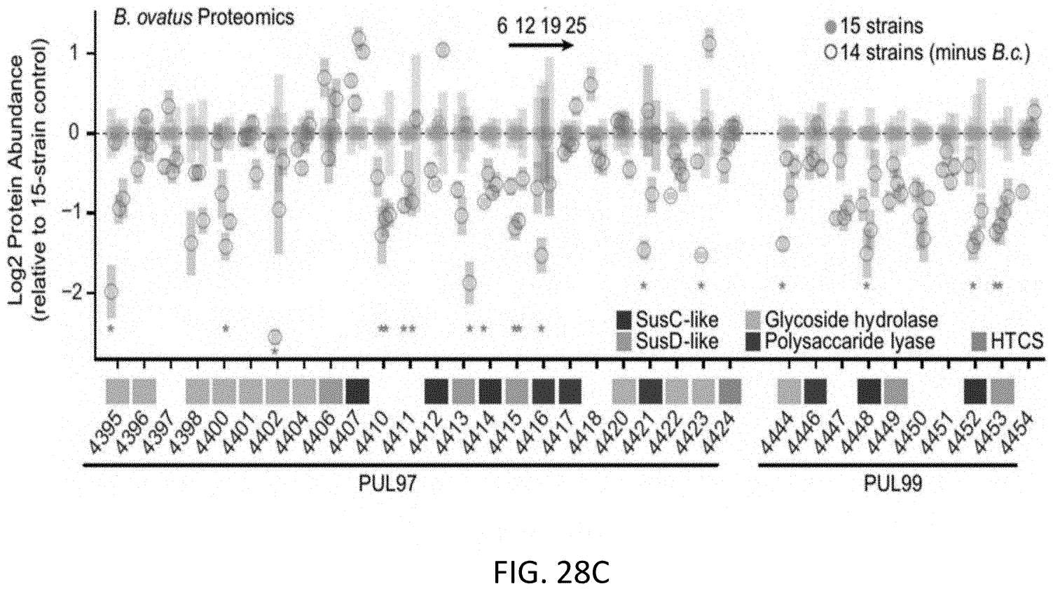

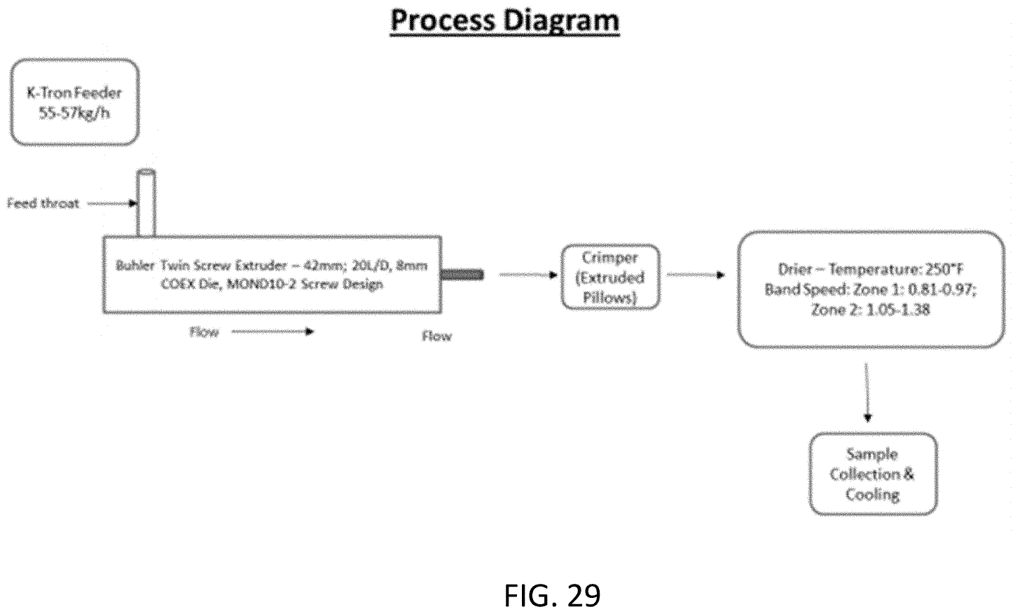

Monosaccharide and linkage analysis verified that arabinoxylan was present in the HiSF-LoFV diet; this conclusion was based on finding abundant 4-linked xylose with branching 4,3-linked xylose, and terminal arabinose (Tables 3-4). We also detected small amounts of 3-linked glucose (indicative of hemicellulose beta-glucans), galacturonic acid and rhamnose. The presence of these structures in the base HiSF-LoFV diet are consistent with the observed increase in abundance of proteins in B. ovatus PULs shown or predicted to process beta-glucan, rhamnogalacturonan, and host glycan when B. cellulosilyticus is present (FIG. 28).

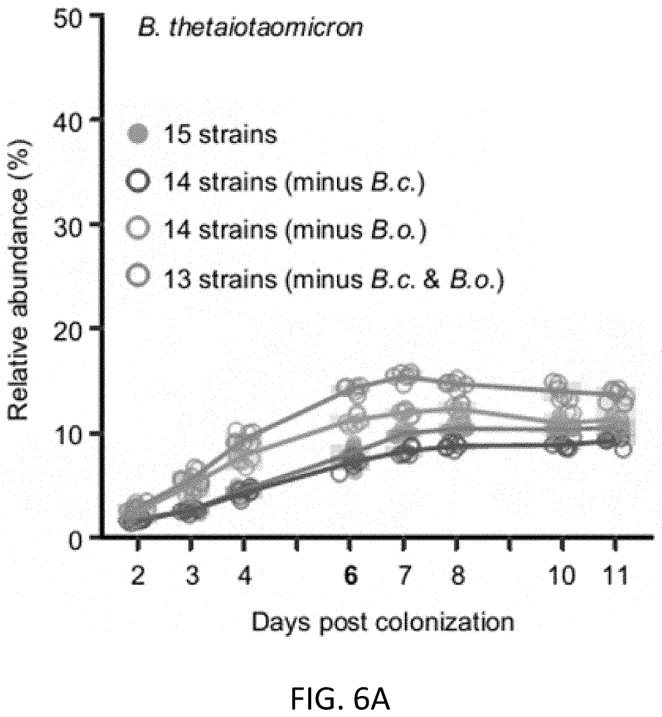

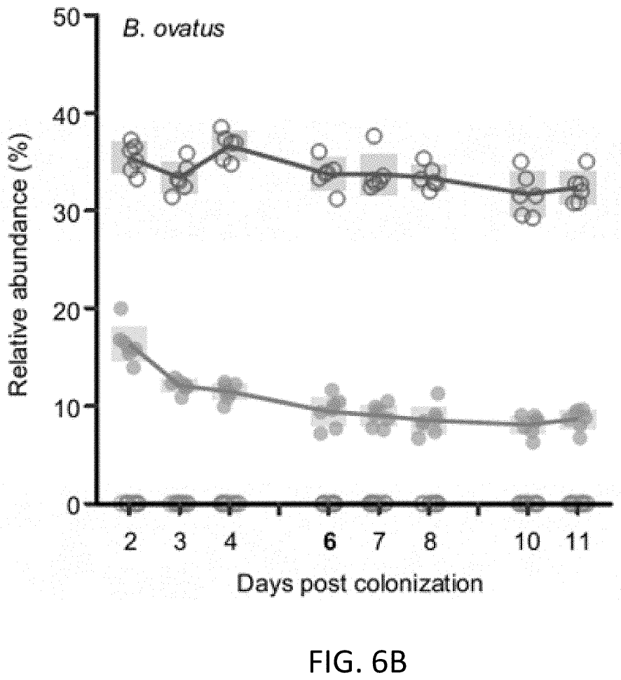

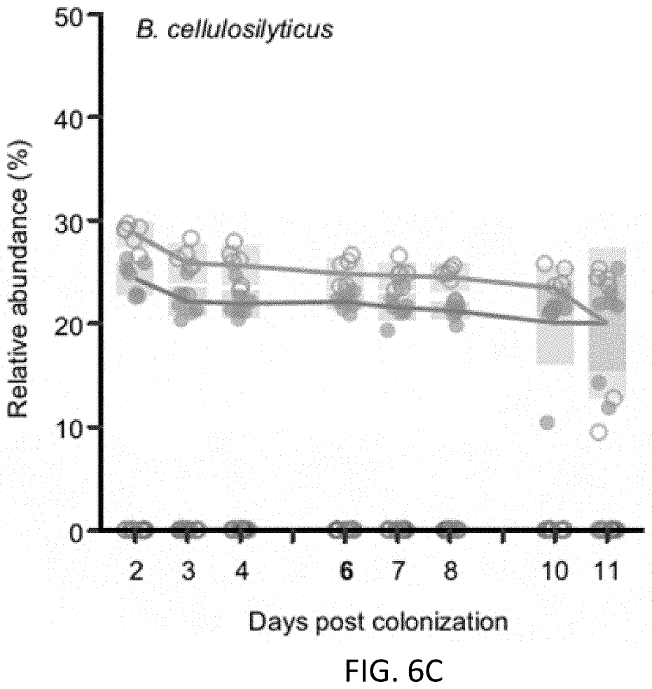

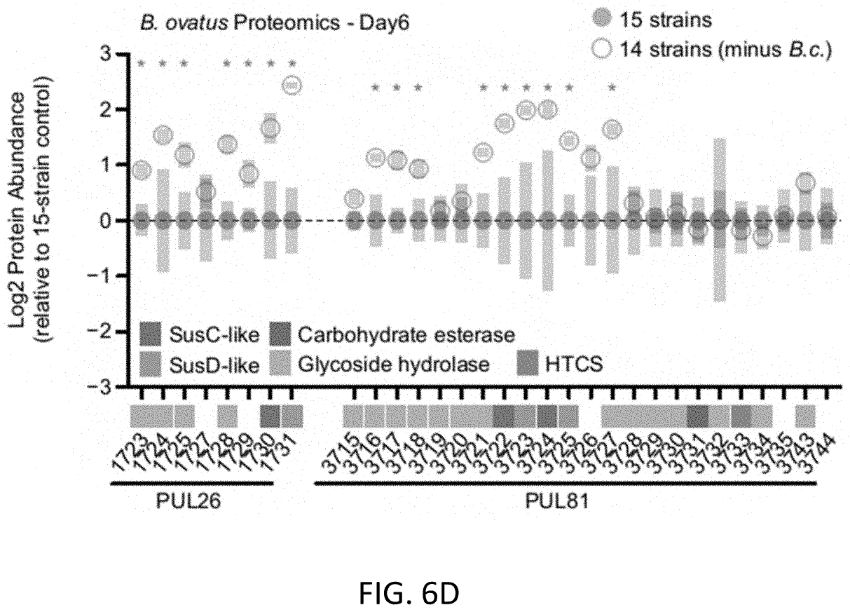

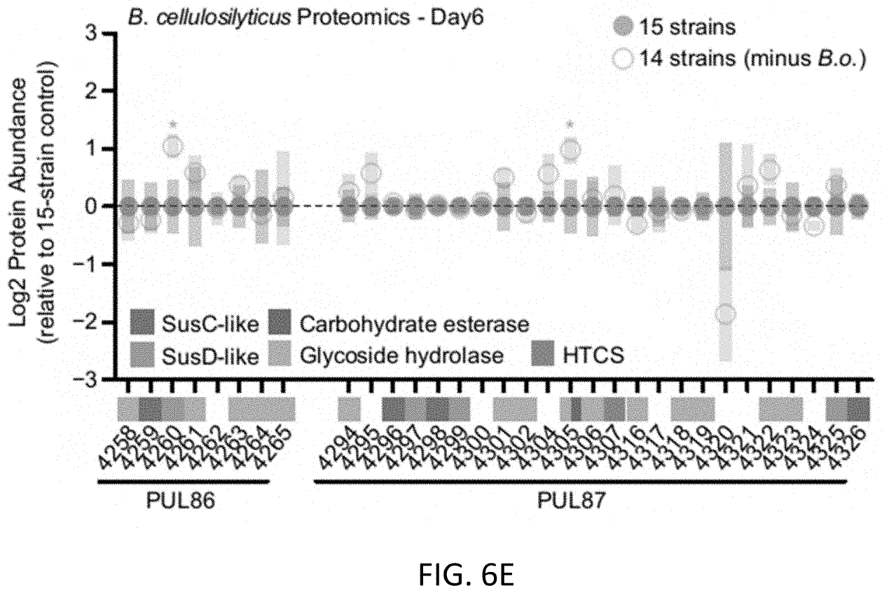

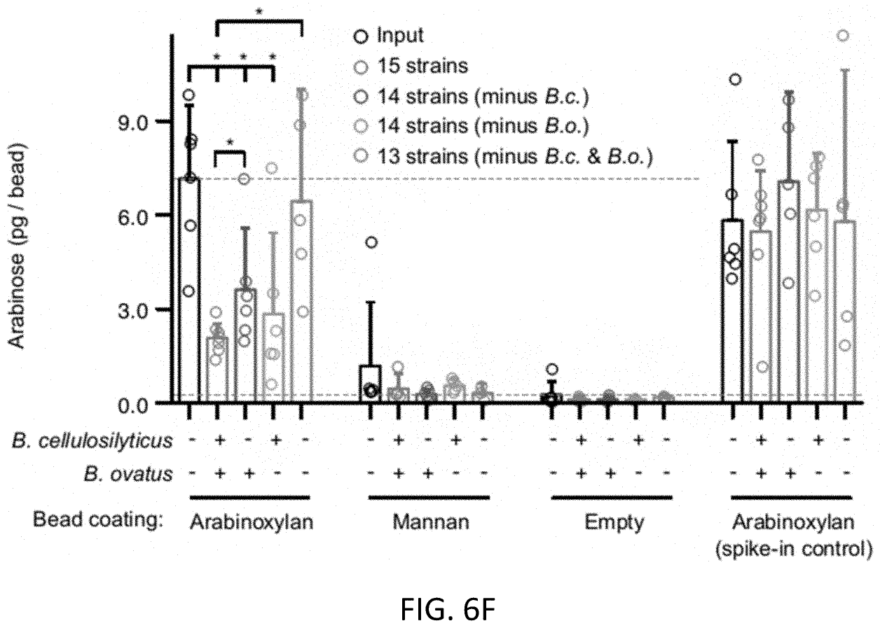

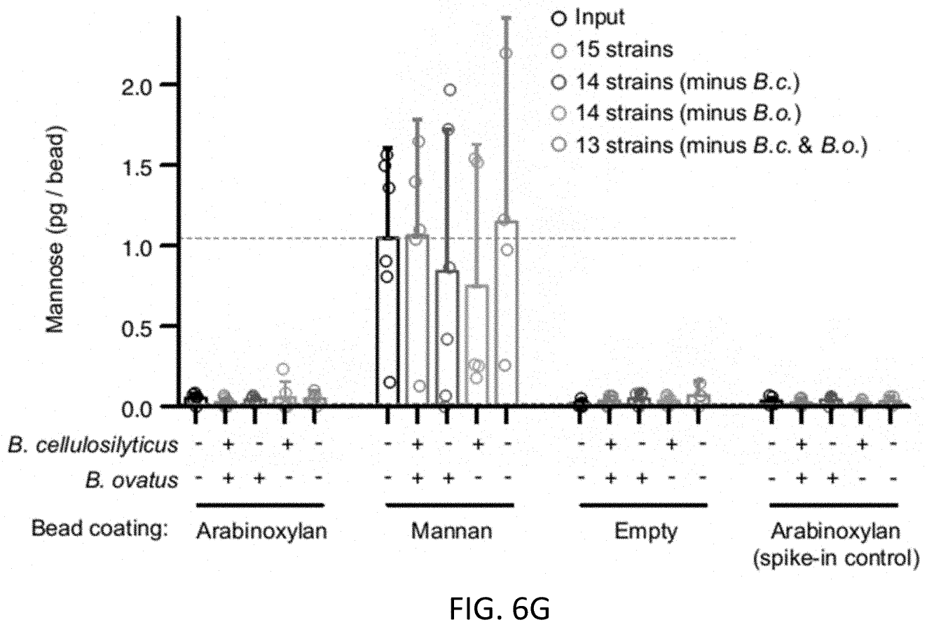

Based on these results, we reasoned that metabolic flexibility allows B. ovatus to acclimate to the presence of B. cellulosilyticus by shifting its nutrient harvesting strategies, de-emphasizing arabinoxylan degradation, thus mitigating competition between the two species. To test this notion further, we performed an experiment omitting B. cellulosilyticus, B. ovatus, or both species from the 15-member consortium introduced into mice (see Tables S4E of Patnode et al., Cell, 2019, 179(1): 59-73). Animals were fed the base HiSF-LoFV diet for 12 days and fecal samples were collected as in previous experiments. Confirming our earlier results, COPRO-Seq revealed that the abundance of B. ovatus was increased in the absence of B. cellulosilyticus (FIG. 6B; see also Tables S4B-E of Patnode et al., Cell, 2019, 179(1): 59-73). Proteomics analysis of fecal samples obtained on day 6 of this experiment also revealed an increase in the abundance of 16 proteins encoded by arabinoxylan-processing PULs 26 and 81 in B. ovatus when B. cellulosilyticus was removed (FIG. 6D). In contrast, the abundance of B. cellulosilyticus as a proportion of the remaining strains did not increase (FIG. 6C; see also Table S4E of Patnode et al., Cell, 2019, 179(1): 59-73), with just one protein specified by each of its arabinoxylan-processing PULs in B. cellulosilyticus (PULs 86 and 87) significantly increasing in abundance when B. ovatus was absent (FIG. 6E). These results, combined with the observation that arabinoxylan-processing genes are important for fitness of B. ovatus only when B. cellulosilyticus is absent (FIG. 5E), indicate that the metabolic flexibility of B. ovatus mitigates competition between two species with the capacity to process the same dietary fiber resource.

We sought to directly measure the functional outcome of metabolic flexibility in B. ovatus and establish that this species degraded arabinoxylan in the community lacking B. cellulosilyticus. Therefore, arabinoxylan-beads, as well as empty and yeast alpha-mannan coated control beads, were administered to the four groups of mice described above, with all mice consuming the base HiSF-LoFV diet. In the absence of B. cellulosilyticus, significant degradation of arabinoxylan was still detected (FIG. 6F), consistent with our previous observations (Tables 8-11). Omission of B. ovatus was also associated with persistent degradation (FIG. 6F), as expected based on the expression of arabinoxylan PULs by B. cellulosilyticus. However, arabinoxylan-coated beads recovered from mice lacking B. ovatus and B. cellulosilyticus were indistinguishable from input beads (FIG. 6F). In addition, omission of both B. cellulosilyticus and B. ovatus did not produce significant increases in the proportions of the remaining strains relative to one another (see Table S4E of Patnode et al., Cell, 2019, 179(1): 59-73), suggesting that these other species were unable to take advantage of the available arabinoxylan resources in the diet. None of the community contexts examined produced significant decreases in bead-bound mannan, controlling for non-specific polysaccharide degradation (FIG. 6G). As an additional ‘spike-in’ control, we added arabinoxylan beads to cecal and fecal samples obtained from all groups of mice immediately after they were euthanized and recovered and processed them in parallel with the orally administered beads. The preservation of carbohydrate on spike-in beads established that B. cellulosilyticus/B. ovatus-dependent degradation occurred during intestinal transit and not sample processing (FIG. 6F).

Together, these experiments show that, in contrast to the persistent competition for arabinan and homogalacturonan exhibited by B. vulgatus, B. ovatus avoids competition for arabinoxylan via acclimation to the presence of its potential competitor, B. cellulosilyticus. This conclusion is based on several observations; (i) the HiSF-LoFV diet contains arabinoxylan polysaccharides, which can be metabolized by both species in question, (ii) omission of B. ovatus did not cause detectable expansion of B. cellulosilyticus, (iii) proteins encoded by B. ovatus arabinoxylan PULs were significantly increased when B. cellulosilyticus was absent, (iv) genes in B. ovatus arabinoxylan PULs were more important for fitness when B. cellulosilyticus was absent, and (v) B. ovatus was responsible for the residual arabinoxylan degradation that took place in the absence of B. cellulosilyticus.

### Example 7—Discussion for Examples 2-6

Together, Examples 2-6 show that, in contrast to the persistent competition for arabinan and homogalacturonan exhibited by B. vulgatus, B. ovatus avoids competition via acclimation to the presence of its potential competitor, B. cellulosilyticus. This conclusion is based on the observations that (i) omission of B. ovatus did not cause detectable expansion of B. cellulosilyticus, (ii) proteins encoded by B. ovatus arabinoxylan PULs were significantly increased when B. cellulosilyticus was absent, (iii) genes in B. ovatus arabinoxylan PULs were significantly more important for fitness when B. cellulosilyticus was absent, and (iv) B. ovatus was responsible for the residual arabinoxylan degradation that took place in the absence of B. cellulosilyticus.

Combining (i) high resolution proteomics, (ii) forward genetic screens for fitness determinants, (iii) a collection of glycan-coated artificial food particles, and (iv) deliberate manipulations of community membership in gnotobiotic mice fed ‘representative’ high-fat, low-fiber USA diet led to the direct characterization of how human gut Bacteroides with distinct, as well as overlapping, nutrient harvesting capacities respond to different food-grade fibers. Our approach allowed us to identify bioactive components in compositionally complex fibers that impact specific members of the microbiota. Obtaining this type of information can inform food manufacturing practices by directing efforts to seek sources of and enrich for these active components; e.g., through judicious selection of cultivars of a given food staple, food processing methods or an existing waste stream from food manufacturing to mine for these components.

Deliberately manipulating membership of a consortium of cultured, sequenced human-donor derived microbes prior to their introduction into gnotobiotic mice fed a human diet, with or without fiber supplementation, provides an opportunity to determine whether and how organisms compete and what mechanisms they use to avoid competition. Simultaneous harvest of a particular dietary resource by two species is theoretically possible whenever they both contain a genetic apparatus sufficient for metabolism of that resource. We provide evidence that competition for particular glycans in fiber preparations is realized in such a model community, since glycan-degrading genes were expressed and required for fitness in both species, and negative interactions were observed in strain omission experiments. These omission experiments disclosed distinct relationships between B. vulgatus, B. ovatus and B. cellulosilyticus; namely, the ability of B. ovatus to acclimate to the presence of a competitor (B. cellulosilyticus) as opposed to the persistent competition between B. vulgatus and B. cellulosilyticus for the same resource. A healthy human gut microbiota has great strain-level diversity. Determining which strains representing a given species to select as a lead candidate probiotic agent, or for incorporation into synbiotic (prebiotic plus probiotic) formulations, is a central challenge for those seeking to develop next generation microbiota-directed therapeutics. Identifying organisms with metabolic flexibility, as opposed to those that are more prone to competing with other community members, could contribute to understanding how certain strains are capable of coexisting with the residents of diverse human gut communities.

Particles present in foods prior to consumption, or generated by physical and biochemical/enzymatic processing of foods during their transit through the gut, provide community members with opportunities to attach to their surfaces, and harvest surface-exposed nutrient resources. The ability of organisms to adhere to such particles, the carrying capacity of particles (size relative to nutrient content), and the physical partitioning their component nutrients can be envisioned as affecting competition, conflict avoidance, and cooperation. The ability of a given gut microbial community to degrade different fiber components was quantified in our studies using artificial food particles composed of fluorescently labeled, paramagnetic microscopic beads coated with different polysaccharides. This approach provides an additional dimension for characterizing the functional properties of a microbial community, and has a number of advantages. First, the measurement of polysaccharides coupled to magnetic beads is not confounded by the presence in the gut of structurally similar (or even identical) dietary or microbial polysaccharides. Second, this technology, when applied to gnotobiotic mice, permits simultaneous testing of multiple glycans in the same animal, allowing a direct comparison of the degradative capabilities of different assemblages of human gut microbes in vivo. For example, we were able to demonstrate non-redundant arabinoxylan degradation carried out by B. cellulosilyticus in this community, despite the presence of another arabinoxylan degrader, B. ovatus. Third, applied directly to humans, these diagnostic biosensors' could be used to quantify functional differences between their gut microbiota, and physical associations between carbohydrates and strains of interest, as a function of host health status, nutritional status/interventions, or other perturbations. As such, results obtained with these biosensors could facilitate ongoing efforts to use machine learning algorithms that integrate a variety of parameters, including biomarkers of host physiologic state and features of the microbiota, to develop more personalized nutritional recommendations (Zeevi et al., 2015). Lastly, this technology could be used to advance food science. The bead coating strategy employed was successful with over 30 commercially available polysaccharide preparations and the assay has been extended to measure the degradation of other biomolecules, including proteins. Particles carrying components of food that have been subjected to different processing methods, or particles bearing combinations of nutrients designed to attract different sets of primary (and secondary) microbial consumers could also be employed in preclinical models to develop and test food prototypes optimized for processing by the microbiota representative of different targeted human consumer populations.

### Example 8—Methods for Examples 2-6

Gnotobiotic mice—All experiments involving mice were carried out in accordance with protocols approved by the Animal Studies Committee of Washington University in St. Louis. For screening different fiber preparations, germ-free male C57BL/6J mice (10-16 weeks-old) were singly housed in cages located within flexible plastic isolators. Cages contained paper houses for environmental enrichment. Animals were maintained on a strict light cycle (lights on at 0600 h, off at 1900 h). Mice were fed a LoSF-HiFV diet for five days prior to colonization. After colonization, the community was allowed to stabilize on the LoSF-HiFV diet for an additional five days. One group of control mice remained on this diet for the rest of the experiment and a second control group was switched to the HiSF-LoFV diet for the rest of the experiment.

Mice in the experimental group first received an introductory diet containing equal parts of all fiber preparations employed in a given screen (totaling 10% of the diet by weight), and then received a series of diets containing different fiber preparations as described in FIG. 1A. A 10 g aliquot of a given diet/fiber mixture was hydrated with 5 mL sterile water in a gnotobiotic isolator; the resulting paste was pressed into a feeding dish and placed on the cage floor. Food levels were monitored nightly, and a freshly hydrated aliquot of that diet was supplied every two days (preventing levels from dropping below roughly one third of the original volume). Bedding (Aspen Woodchips; Northeastern Products) was replaced after each 7-day diet period to prevent any spilled food from being consumed during the next diet exposure. Fresh fecal samples were collected from each animal within seconds of being produced on days 1, 3, 6, and 7 of every diet period, and placed in liquid nitrogen within 45 min. Pre-colonization fecal samples were collected to verify the germ-free status of mice.

For monotonous feeding experiments, mice were fed the control HiSF-LoFV diet in its pelleted form for two weeks prior to colonization. Two days after colonization, mice were switched to paste diets containing 10% of the powdered fiber preparation mixed into the base diet (or the base diet in paste form without added fiber) for the remainder of the experiment. As noted above, these diets were delivered in freshly hydrated aliquots every two days. Fecal samples, including those obtained prior to colonization, were collected on the days indicated in FIG. 11.

Defined microbial communities—The screening experiments used cultured, sequenced bacterial strains obtained from a fecal sample that had been collected from a lean co-twin in an obesity-discordant twin-pair [Twin Pair 1 in (Ridaura et al., 2013); also known as F60T2 in (Faith et al., 2013)]. Isolates were grown to stationary phase in TYGS medium (Goodman et al., 2009) in an anaerobic chamber (atmosphere; 75% N2, 20% CO2, 5% H2). Equivalent numbers of organisms were pooled (based on OD600 measurements). The pool was divided into aliquots that were frozen in TYGS/15% glycerol, and maintained at −80° C. until use. On experimental day 0, aliquots were thawed, the outer surface of their tubes were sterilized with Clidox (Pharmacal) and the tubes were introduced into gnotobiotic isolators. The bacterial consortium was administered through a plastic tipped oral gavage needle (total volume, 400 μL per mouse). Based on inconsistent colonization observed in screening experiment 1 (see Table S1A of Patnode et al., Cell, 2019, 179(1): 59-73), one isolate (Enterococcus fecalis; average relative abundance, 2.1%) was not included in screening experiments 2 and 3.

Model communities containing INSeq libraries—Ten strains selected from the human donor-derived community described above were colony purified, and each frozen in 15% glycerol and TYGS medium. Recoverable CFUs/mL were quantified by plating on brain-heart-infusion (BHI) blood agar. The identity of strains was verified by sequencing full-length 16S rRNA amplicons. On the day of gavage, stocks of these strains were thawed in an anaerobic chamber and mixed together along with each of five multi-taxon INSeq libraries (B. thetaiotaomicron VPI-5482, B. thetaiotaomicron 7330, B. cellulosilyticus WH2, B. vulgatus ATCC-8482, B. ovatus ATCC-8483) whose generation and characterization have been described in earlier publications (Hibberd et al., 2017; Wu et al., 2015). An aliquot of this mixture was administered by oral gavage to germ-free mice housed in gnotobiotic isolators (2×10⁶ CFUs of each donor organism plus an OD600 0.5 of each INSeq library per mouse recipient; total gavage volume, 400 μL). For B. cellulosilyticus, B. vulgatus, B. ovatus, or B. cellulosilyticus and B. ovatus omission experiments, gavage mixtures were prepared in parallel without these organisms. The absence of one or both of these strains was verified by COPRO-Seq analysis of both the gavage mixture and fecal samples collected throughout the experiment from recipient mice.

Fiber-rich food ingredient mixtures—HiSF-LoFV and LoSF-HiFV diets were produced using human foods, selected based on consumption patterns from the National Health and Nutrition Examination Survey (NHANES) database (Ridaura et al., 2013). Diets were milled to powder (D90 particle size, 980 μm), and mixed with pairs of powdered fiber preparations [one preparation at 8% (w/w) and the other preparation at 2% (w/w)]. Fiber content was defined for each preparation [Association of Official Agricultural Chemists (AOAC) 2009.01], as was protein, fat, total carbohydrate, ash, and water content [protein AOAC 920.123; fat AOAC 933.05; ash AOAC 935.42; moisture AOAC 926.08; total carbohydrate (100−(Protein+Fat+Ash+Moisture)]. The powdered mixtures were sealed in containers and sterilized by gamma irradiation (20-50 kilogreys, Steris, Mentor, Ohio). Sterility was confirmed by culturing the diet under aerobic and anaerobic conditions (atmosphere, 75% N2, 20% CO₂, 5% H₂) at 37° C. in TYG medium, and by feeding the diets to germ-free mice followed by COPRO-Seq analysis of their fecal DNA.

Monosaccharide and linkage analysis of fiber preparations—For fiber preparations, uronic acid (as GalA) was measured using the m-hydroxybiphenyl method (Thibault, 1979). Sodium tetraborate was used to distinguish GlcA and GalA (Filisetti-Cozzi and Carpita, 1991). The degree of methylation of galacturonic acid (pectins) in the sample was estimated as previously described (Levigne et al., 2002). Samples were hydrolyzed with 1M H₂SO₄ for 2 h at 100° C. and individual neutral sugars were analyzed as their alditol acetate derivatives (Englyst and Cummings, 1988) by gas chromatography. To fully release glucose from cellulose, a pre-hydrolysis step was carried out by incubation in 72% H₂SO₄ for 30 minutes at 25° C. prior to the hydrolysis step. Linkage analysis was performed after carboxyl reduction of uronic acid with NaBD4/NaBH4 according to a previously published procedure (Pettolino et al., 2012) with minor modifications (this procedure allows galactose, galacturonic acid and methylesterified galacturonic acid to be distinguished). Methylation of carboxyl-reduced samples was performed as described in (Buffetto et al., 2015).

Polysaccharides from the HiSF-LoFV diet were isolated by sequential alkaline extractions (Pattathil et. al., 2012). Briefly, lipids were removed from a sample of powdered HiSF-LoFV by sequential incubation in 80% ethanol, 100% ethanol, and acetone. The dried precipitate was suspended in 1M KOH containing 0.5% (w/w) NaBH4 and stirred overnight. The solution was neutralized and the supernatant was collected by centrifugation (this material is referred to as fraction 1 (F1)). The insoluble material was suspended in 1M KOH/0.5% (w/w) NaBH4 overnight, and the supernatant was collected (referred to as F2). The insoluble material was suspended in 4M KOH/0.5% (w/w) NaBH4 overnight and the supernatant was collected (referred to as F3). Each fraction was dialyzed (SnakeSkin 3.5K MWCO, Thermo Scientific) in water, lyophilized, and then treated for 4 hours at 37° C. with amyloglucosidase (36 units/mg) and alpha-amylase (100 units/mg; both enzymes from Megazyme). Enzymes were inactivated by boiling and samples were dialyzed and lyophilized. Measurement of the dry mass of each fraction before and digestion revealed that the total starch content of the base HiSF-LoFV diet was 22% (w/w) (note a comparable analysis the pea fiber yielded a value of 3.6%, meaning that HiSF-LoFV diet supplemented with 10% pea fiber contains a total starch content of 20% by weight).

HiSF-LoFV diet polysaccharides were analyzed by the Center for Complex Carbohydrate Research at the University of Georgia in Athens. Glycosyl composition analysis was performed by combined GC-MS of the per-O-trimethylsilyl (TMS) derivatives of the monosaccharide methyl glycosides produced from the sample by acidic methanolysis (Santander et al., 2013). Briefly, samples (300-500 μg) were heated with methanolic HCl in a sealed screw-top glass test tube for 17 h at 80° C. After cooling and removal of the solvent under a stream of nitrogen, samples were derivatized with Tri-Sil® (Pierce) at 80° C. for 30 min. GC-MS analysis of the TMS methyl glycosides was performed on an Agilent 7890A GC interfaced to a 5975C mass selective detector (MSD), using a Supelco Equity-1 fused silica capillary column (30 m×0.25 mm ID).

Glycosyl-linkage analysis of HiSF-LoFV diet polysaccharides was performed as previously described with slight modification (Heiss et. al., 2009). Samples were permethylated, depolymerized, reduced and acetylated, and the resulting partially methylated alditol acetates (PMAAs) were analyzed by GC-MS. About 1 mg of the sample was used for linkage analysis. The sample was suspended in 200 μL of dimethyl sulfoxide and left to stir for 1 day. Permethylation of the sample was affected by two rounds of treatment with sodium hydroxide (15 minutes) and methyl iodide (45 minutes). The permethylated material was hydrolyzed using 2 M TFA (2 hours in sealed tube at 121° C.), reduced with NaBD4, and acetylated using acetic anhydride/TFA. The resulting PMAAs were analyzed on an Agilent 7890A GC interfaced to a 5975C MSD (electron impact ionization mode); separation was performed on a 30 m Supelco SP-2331 bonded phase fused silica capillary column.

V4-16S rRNA gene sequencing—DNA was isolated from fecal samples by first bead-beating the sample with 0.15 mm-diameter zirconium oxide beads and a 5 mm-diameter steel ball in 2× buffer A (200 mM NaCl, 200 mM Tris, 20 mM EDTA), followed by extraction in phenol:chloroform:isoamyl alcohol, and further purification (QiaQuick 96 purification kit; Qiagen, Valencia, Calif.). PCR amplification of the V4 region of bacterial 16S rRNA genes was performed as described (Bokulich et al., 2013). Amplicons with sample-specific barcodes were pooled for multiplex sequencing using an Illumina MiSeq instrument. Reads were demultiplexed and rarefied to 5000 reads per sample. Reads sharing≥99%, nucleotide sequence identity [99% ID operational taxonomic units (OTUs)], that mapped to a reference OTU in the GreenGenes 16S rRNA gene database (McDonald et al., 2012) were assigned to that OTU. The 16S rRNA gene could not be amplified in multiple fecal DNA samples from mice fed 8% cocoa fiber. A small subset of reads (<5%) representing additional V4-16S rDNA amplicon sequences produced from colony-purified stocks of Bacteroides ovatus, Parabacteroides distasonis, Dorea longicatena, and Collinsella aerofaciens were omitted from our analyses of fecal DNA samples. Streptococcus thermophilus, an organism heavily used in cheese processing, was also omitted based on its detection in DNA isolated from samples of the sterile HiSF-LoFV diet.

COPRO-Seq analyses of bacterial species abundances—Libraries were prepared from fecal DNA using sonication and addition of paired-end barcoded adaptors (McNulty et al., 2013) or by tagmentation using the Nextera DNA Library Prep Kit (Illumina) and combinations of custom barcoded primers (Adey et al., 2010). Libraries were sequenced using an Illumina NextSeq instrument [1,011,017±314,473 reads/sample (mean±SD) across experiments]. Reads were mapped to bacterial genomes with previously published custom Perl scripts (see below) adapted to use Bowtie II for genome alignments (Hibberd et al., 2017); samples represented by less than 150,000 uniquely mapped reads were omitted from the analysis.

Community-wide quantitative proteomics—Lysates were prepared from fecal samples by bead beating in SDS buffer (4% SDS, 100 mM Tris-HCl, 10 mM dithiothreitol, pH 8.0) using 0.15 mm diameter zirconium oxide beads, followed by centrifugation at 21,000×g for 10 minutes. Pre-cleared protein lysates were further denatured by incubation at 85° C. for 10 minutes, and adjusted to 30 mM iodoacetamide to alkylate reduced cysteines. After incubation in the dark for 20 minutes at room temperature, protein was isolated by chloroform-methanol extraction. Protein pellets were then washed with methanol, air dried, and re-solubilized in 4% sodium deoxycholate (SDC) in 100 mM ammonium bicarbonate (ABC) buffer, pH 8.0. Protein concentrations were measured using the BCA (bicinchoninic acid) assay (Pierce). Protein samples (250 □g) were then transferred to a 10 kDa MWCO spin filter (Vivaspin 500, Sartorius), concentrated, rinsed with ABC buffer, and digested in situ with sequencing-grade trypsin (Clarkson et al., 2017). The tryptic peptide solution was then passed through the spin-filter membrane, adjusted to 1% formic acid to precipitate the remaining SDC, and the precipitate removed from the peptide solution with water-saturated ethyl acetate. Peptide samples were concentrated using a SpeedVac, measured by BCA assay and analyzed by automated 2D LC-MS/MS using a Vanquish UHPLC with autosampler plumbed directly in-line with a Q Exactive Plus mass spectrometer (Thermo Scientific) outfitted with a 100 μm ID triphasic back column [RP-SCX-RP; reversed-phase (5 μm Kinetex C18) and strong-cation exchange (5 μm Luna SCX) chromatographic resins; Phenomenex] coupled to an in-house pulled, 75 μm ID nanospray emitter packed with 30 cm Kinetex C18 resin. For each sample, 12 μg of peptides were autoloaded, desalted, separated and analyzed across four successive salt cuts of ammonium acetate (35, 50, 100 and 500 mM), each followed by a 105-minute organic gradient. Eluting peptides were measured and sequenced by data-dependent acquisition on the Q Exactive Plus (Clarkson et al., 2017).

MS/MS spectra were searched with MyriMatch v.2.2 (Tabb et al., 2007) against a proteome database derived from the genomes of the strains in the defined model community concatenated with major dietary protein sequences, common protein contaminants, and reversed entries to estimate false-discovery rates (FDR). Since the relative abundance of B. thetaiotaomicron 7330 was low on day 6 [0.05%±0.041% (mean±SD) for all groups], we chose to analyze all peptides that mapped to the B. thetaiotaomicron VPI-5482 proteome, regardless of whether they also mapped to B. thetaiotaomicron 7330. Peptide spectrum matches (PSM) were required to be fully tryptic with any number of missed cleavages, and contain a static modification of 57.0214 Da on cysteine and a dynamic modification of 15.9949 Da on methionine. PSMs were filtered using IDPicker v.3.0 (Ma et al., 2009) with an experiment-wide FDR<1% at the peptide-level. Peptide intensities were assessed by chromatographic area-under-the-curve (label-free quantification option in IDPicker). To remove cases of extreme sequence redundancy, the community meta-proteome was clustered at 100% sequence identity post-database search [UCLUST; (Edgar, 2010)] and peptide intensities were summed to their respective protein groups/seeds to estimate overall protein abundance. Proteins were included in the analysis only if they were detected in more than 3 biological replicates in at least one experimental group. Missing values were imputed to simulate the limit of detection of the mass spectrometer, using mean minus 2.2×standard deviation with a width of 0.3×standard deviation. Four additional imputed distributions produced results that were in general agreement with this approach in terms of fold-abundance change induced by fiber treatment and statistical significance.

Multi-taxon INSeq—Multi-taxon INSeq allows simultaneous analysis of multiple mutant libraries in the same recipient gnotobiotic mouse owing to the fact that the mariner Tn vector contains Mmel sites at each end plus taxon-specific barcodes. Mmel digestion cleaves genomic DNA at a site 20-21 bp distal to the restriction enzyme's recognition site so that the site of Tn insertion and the relative abundance of each Tn mutant can be defined in given diet/community contexts by sequencing the flanking genomic sequence and taxon-specific barcode (Wu et al., 2015). Purified fecal DNA was processed as described previously (Wu et al., 2015). DNA was digested with Mmel and the products were ligated to sample-specific barcoded adaptors. Sequencing was performed on an IIlumina HiSeq 2500 instrument, with a custom indexing primer providing the strain-specific barcode for the insertion. Analysis of mutant strain frequencies was carried out using custom software. Log ratios of the abundances of Tn mutant strains on experimental days 6 and 2 (corresponding to the period of fiber treatment compared to just prior to fiber exposure) were calculated for each mouse.

PUL nomenclature and homology—All PUL assignments were made based on “new assembly” genomes present in the CAZy PUL database (www.cazy.org/PULDB) (Terrapon et al., 2018). All boundaries of PULs were algorithmically defined (listed as ‘predicted PUL’ in PULDB). The algorithmically defined boundaries of B. thetaiotaomicron PUL7 were extended to include the adjacent arabinose operon based on previously published experimental datasets (Schwalm et al., 2016). A cluster of three or more adjacent CAZymes was defined as a ‘polysaccharide utilization complement’. Homology between genes in PULs was determined using a reciprocal BLASTp approach with an E-value threshold of 1×10⁻⁹, querying each protein product contained within a CAZy-annotated PUL against reference genomes from other species in the community.

Generation of glycan-coated magnetic beads—Wheat Arabinoxylan and Icelandic Moss Lichenan were purchased from Megazyme (P-WAXYL, P-LICHN) and yeast alpha-mannan was purchased from Sigma-Aldrich (M7504). Polysaccharides were solubilized in water (at a concentration of 5 mg/mL for pea fiber and 20 mg/mL for arabinoxylan and lichenan), sonicated and heated to 100° C. for 1 minute, then centrifuged at 24,000×g for 10 minutes to remove debris. TFPA-PEG3-biotin (Thermo Scientific), dissolved in DMSO (10 mg/mL) was added to the polysaccharide solution at a ratio of 1:5 (v/v). The sample was subjected to UV irradiation for 10 minutes (UV-B 306 nm, 7844 mJ total), and then diluted 1:4 to facilitate desalting on 7 kD Zeba spin columns (Thermo Scientific).

Biotinylated polysaccharide was mixed with one of several biotinylated fluorophores (PF-505, PF-510LSS, PF-633, PF-415; all at a concentration of 50 ng/mL; all obtained from Promokine). A 500 μL aliquot of this preparation was incubated with 10⁷ paramagnetic streptavidin-coated silica beads (LSKMAGT, Millipore Sigma) for 24 hours at room temperature. Beads were washed by centrifugation three times with 1 mL HNTB buffer (10 mM HEPES, 150 mM NaCl, 0.05% Tween-20, 0.1% BSA) followed by addition of 5 μg/mL streptavidin (Jackson Immunoresearch) in HNTB (30 min incubation at room temperature). Beads were washed as before and then incubated with 250 μL of the biotinylated polysaccharide preparation. The washing, streptavidin, and polysaccharide incubation steps were repeated three times. Bead preparations were assessed using an Aria III cell sorter (BD Biosciences) to confirm adequate labeling, and then analyzed by GC-MS (see below) to quantify the amount of carbohydrate bound.

Administration and recovery of beads—Beads were incubated with 70% ethanol for 1 minute in a biosafety cabinet, then washed three times with 1 mL sterile HNTB using a magnetic stand. The different bead types were combined, diluted, and aliquoted to 10⁷ beads per 650 μL HNTB insterile Eppendorf microcentrifuge tubes. The number of beads in each aliquot was counted using an Aria III cell sorter and CountBright fluorescent microspheres (BD Bioscience). Tubes containing beads were introduced into gnotobiotic isolators and the beads were administered by oral gavage (600 μL per mouse). Separate aliquots of control beads, used to establish input carbohydrate content were stored in the dark at 37° C. until collection of experimental beads from mouse fecal or cecal samples had been completed.

For germ-free mouse experiments, animals were fed the HiSF-LoFV diet for two weeks and then gavaged with beads; all fecal pellets were collected during the 4- to 12-hour interval that followed gavage. During this time period, bedding was removed and mice were placed on grated cage bottoms (with access to food and water); cage bottoms were placed just above a 0.5 cm deep layer of sterile water on the floor of the cage, to prevent pellets from drying. For colonized animals, cecal and colonic contents were collected four hours after administration of beads at the time of euthanasia. Recovered samples were immediately placed in sterile water on ice.

Fecal, cecal, and input samples were vortexed and filtered through nylon mesh (100 μm pore-diameter). The resulting suspension of luminal contents was layered over sterile Percoll Plus (GE Health Care) and centrifuged for 5 minutes at 500×g. Beads were collected from underneath the Percoll layer and washed four times using a magnetic stand, each time with 1 mL fresh HNTB. Recovered beads were counted by flow cytometry as before, filtered through nylon mesh (40 μm pore diameter, BD Biosciences) and stored at 4° C. overnight. Beads were sorted back into their polysaccharide types based on fluorescence using an Aria III sorter (average sort purity, 96%). Sorted samples were centrifuged (500×g for 5 minutes) to pellet beads and the beads were transferred to a 96-well plate. All bead samples were incubated with 1% SDS/6M Urea/HNTB for 10 minutes at room temperature to remove exogenous components, washed three times with 200 μL HNTB using a magnetic plate rack, and then stored overnight at 4° C. prior to monosaccharide analysis.

Analysis of bead-bound glycan by GC-MS—The number and purity of beads in each sorted sample was determined by taking an aliquot for analysis on the Aria III cell sorter. Equal numbers of beads from each sample were transferred to a new 96-well plate and the supernatant was removed with a magnetic plate rack. For acid hydrolysis, 200 μL of 2M trifluoroacetic acid and 250 ng/mL myo-inositol-D6 (CDN Isotopes; spike-in control) were added to each well, and the entire volume was transferred to 300 μL glass vials (ThermoFisher; catalog number C4008-632C). Another aliquot was taken to verify the final number of beads in each sample. Monosaccharide standards were included in separate wells and subjected to the hydrolysis protocol in parallel with the other samples. Vials were crimped with Teflon-lined silicone caps (ThermoFisher) and incubated at 100° C. with rocking for 2 h. Vials were then cooled, spun to pellet beads, and their caps were removed. A 180 μL aliquot of the supernatant was collected and transferred to new 300 μL glass vials. Samples were dried in a SpeedVac for 4 hours, methoximated in 20 μL O-methoxyamine (15 mg/mL pyridine) for 15 h at 37° C., followed by trimethylsilylation in 20 μL MSTFA/TMCS [N-Methyl-N-trimethylsilyltrifluoroacetamide/2,2,2-trifluoro-N-methyl-N-(trimethylsilyl)-acetamide, chlorotrimethylsilane] (ThermoFisher) for 1 h at 70° C. One half volume of heptane (20 μL) was added before loading the samples for injection onto a 7890B gas chromatography system coupled to a 5977B MS detector (Agilent). The mass of each monosaccharide detected in each sample of sorted beads was determined using monosaccharide standard curves. This mass was then divided by the final count of beads in each sample to produce a measurement of mass of recoverable monosaccharide per bead.

Quantification and Statistical Analysis—Using data from days 6 and 7 of each diet treatment, a mixed effects model was generated in the R programming environment for each species in each of three fiber screening experiments. The relative abundance of that species in feces (or the relative abundance scaled by fecal DNA yield) was used as the dependent variable, and the concentration of administered fiber (10 to 13 fibers tested per experiment), as well as experimental day were used as independent variables. Mixed effects models incorporated terms to describe repeated measures of individual mice. In rare cases where B. cellulosilyticus failed to colonize (5 of 60 mice), the animals were not considered biological replicates since they harbored a distinct microbiota; they were omitted from the models. ANOVA (with Satterthwaite approximation for degrees of freedom) was performed to evaluate the significance of individual terms in models (FDR corrected P value cutoff of 0.01). Models were evaluated based on conditional R² values (incorporating random factors) and plots of the residuals and Cook's distance (no samples were excluded based on these assessments).

For COPRO-Seq analyses, differences between groups were assessed using mixed-effect models with time as a categorical variable, including day 2 as a pre-treatment time point. For omission experiments, the abundance of each strain as a proportion of all other strains except the omitted strain or strains was used for statistical tests. Significant terms in models were identified using ANOVA (FDR corrected P value cutoff of 0.05). Mann-Whitney U test was used for analyses of individual time-points of interest.

For quantitative proteomics, significant differences in protein abundance were determined using limma (Ting et al., 2009). For multi-taxon INSeq analyses, mutant strain abundances were analyzed using limma-voom (Law et al., 2014) after quantile normalization. The general linear model framework in limma-voom allowed us to perform moderated t-tests to determine the statistical significance (P<0.05, FDR corrected) of differences in fitness in the context of the control versus fiber-supplemented diets. A Mann-Whitney U test was used to calculate significant differences in monosaccharide abundance between bead samples. All tests were two-tailed.

Data and Software Availability—Datasets of V4-16S rRNA sequences in raw format prior to post-processing and data analysis, plus COPRO-Seq and INSeq datasets have been deposited at the European Nucleotide Archive under study accession PRJEB26564. All LC-MS/MS proteomic data have been deposited into the MassIVE data repository under accession numbers MSV000082287 (MassIVE) and PXD009535 (ProteomeXchange). INSeq software: github.com/mengwu1002/Multi-taxon_analysis_pipeline. COPRO-Seq software: github.com/nmcnulty/COPRO-Seq.

### Example 9—Sugar Beet Arabinan Degradation

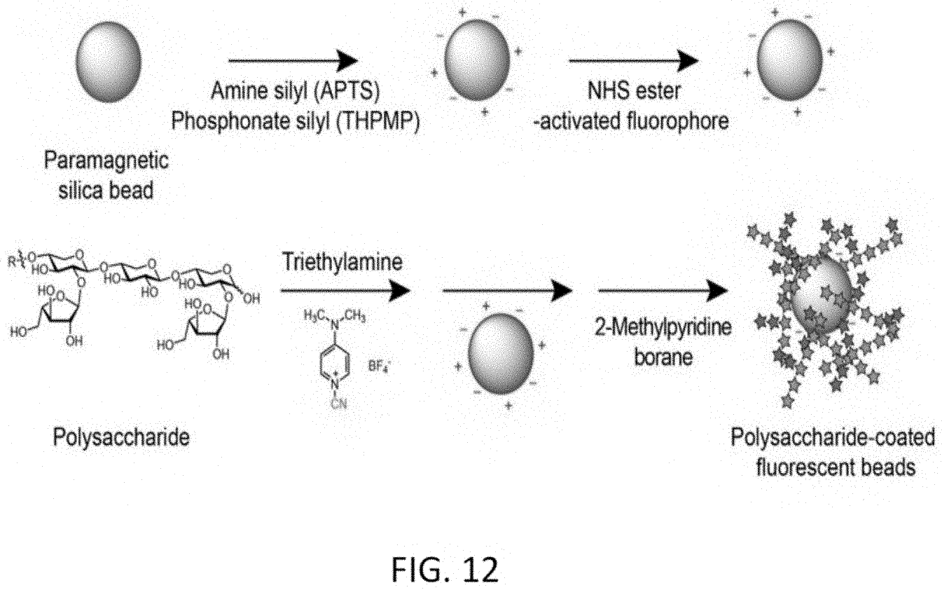

This example describes an alternative method used to attach polysaccharides to paramagnetic glass beads. To covalently immobilize polysaccharides onto paramagnetic glass beads for use as biosensors of gut microbiota biochemical function, a bead with unique chemical functionality was developed. Amine functional groups were added to the bead surface as a chemical handle because of their nucleophilic nature at neutral pH and their utility in multiple bioconjugation reactions (Koniev et al., 2015). It was hypothesized that the amine functional group could be used for two critical functions: 1) addition of a fluorophore for the multiplexed analysis of multiple bead types within a single animal or subject, and 2) the covalent immobilization of an activated polysaccharide (FIG. 12).

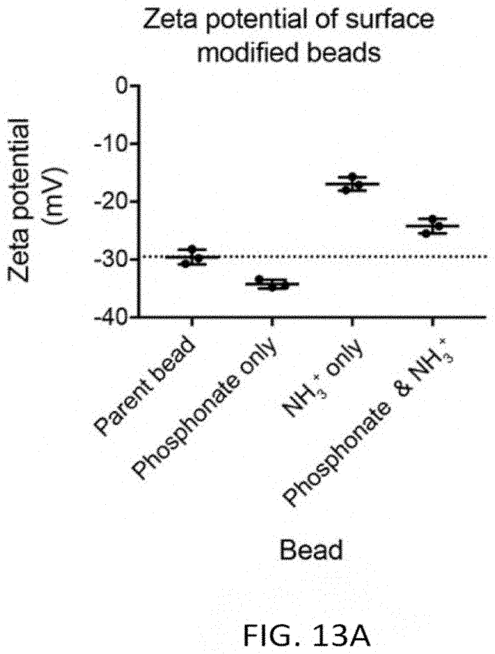

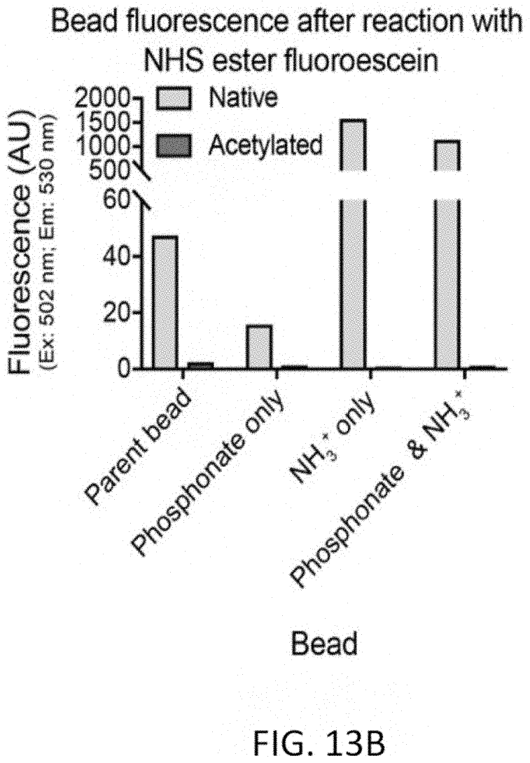

To install amines on the bead surface, the activated amine-silyl reagent (3-aminopropyl)triethoxysilane (ATPS) was reacted with bead in the presence of water. Under the same reaction conditions, a zwitterionic surface could be generated with 3-(trihydroxysilyl)propyl methylphosphonate (THPMP) to an ATPS containing reaction. The additional phosphonate functionality was important to reduce nonspecific binding to the bead surface (Bagwe et al., 2006). The zeta potential of surface modified paramagnetic silica beads was used to monitor the addition of both amine and phosphonate functional groups onto the bead surface (FIG. 13A).

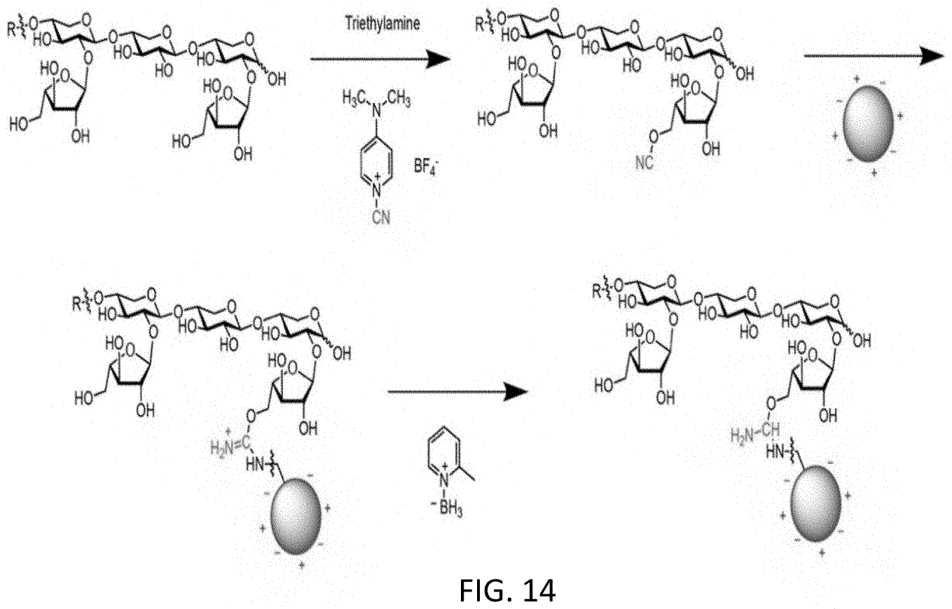

With fluorescent amine-phosphonate paramagnetic glass beads in hand, we next sought to covalently immobilize polysaccharides of interest of the bead surface. Strategies for bioconjugation with polysaccharides are lacking compared to proteins, peptide, and nucleic acids due to the limited chemical functionality naturally occurring within polysaccharides. We chose to activate polysaccharides using a cyano (CN—) donor to generate a cyano-ester. Suitable cyano-donors include, but are not limited to, cyanogen bromide (CNBr) (Glabe et al., 1983) and the organic nitrile donor 1-cyano-4-dimethylam inopyridinium tetrafluoroborate (CDAP) (Lees et al., 1996). Both donors have been used for the generation of affinity matrixes on agarose beads and the synthesis of polysaccharide-conjugate vaccines; specifically, CDAP activation and conjugation was used for the development of the pneumococcal-conjugate vaccines (Lees et al., 1996; Ridaura et al., 2013). We chose CDAP because of its solubility in DMSO and the fact that it is less pH sensitive and less toxic than CNBr. CDAP was dissolved in DMSO and added to a solution of polysaccharide in the presence of catalytic triethylamine. CDAP nonspecifically generates cyano-ester electrophiles from the hydroxyls naturally present within a polysaccharide (FIG. 14). After activation, fluorescent amine-phosphonate beads were added. The solution was allowed to react overnight. Reaction of bead surface amine and the cyano-ester group of the activated polysaccharide yields a liable isourea bond that is reduced to a stable covalent bond with the addition of a hydride donor. We chose 2-methylpyridine borane although harsher donors such as sodium borohydride or sodium cyanoborohydride will also work. Immobilization of polysaccharide on the bead surface and reduction of the isourea bond has little to no effect on bead fluorescence.

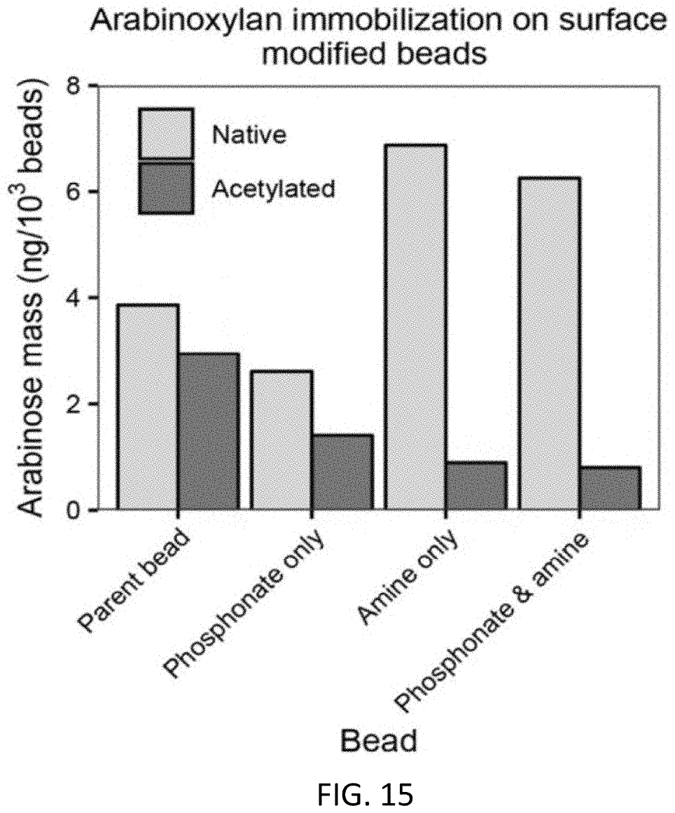

Polysaccharide immobilization on the bead surface was quantified via acid hydrolysis of surface-immobilized polysaccharide and quantification of the liberated monosaccharides using gas chromatography mass spectrometry (GC-MS). Polysaccharide was hydrolyzed using 2 M trifluoroacetic acid and liberated monosaccharide were quantified on as silylated methoxyamine-reduced monosaccharides using free monosaccharides as standards. Beads were enumerated with flow cytometry and an equal number of each bead type were assayed in parallel. Beads lacking surface amines, or beads reacted with polysaccharides not activated with CDAP lacked surface-immobilized polysaccharide (FIG. 15). Typical bead yields are 5-25 pg of immobilized polysaccharide per bead.

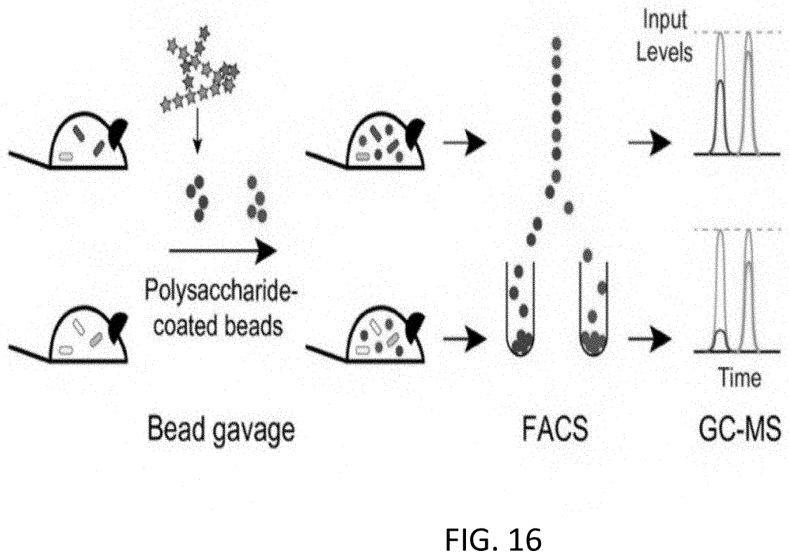

Multiple types of polysaccharide-coated beads labeled with distinct fluorophores were pooled and gavaged into gnotobiotic mouse models as biosensors of gut community biochemical function. Polysaccharide degradation was measured as a function of 1) community composition, and 2) diet. Pooled beads were gavaged into germ-free mice 4 hours prior to animals were euthanized; beads were subsequently isolated from the mouse cecum based on their density and magnetic properties. Polysaccharide degradation was quantified as the amount of polysaccharide remaining covalently bound to the bead after passage through the gut and recovery from the cecum (FIG. 16).

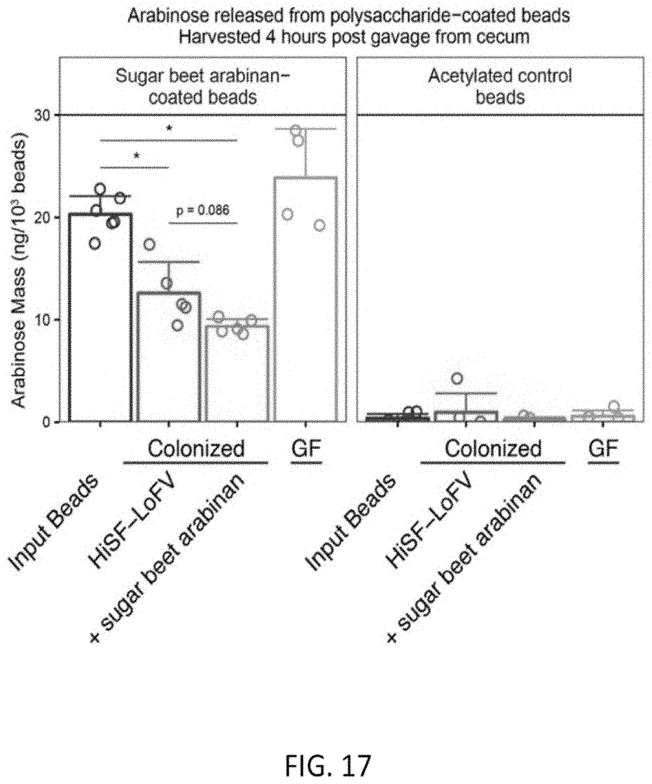

The ability of a microbiota to degrade a commercially available preparation of sugar beet arabinan (Megazyme; cat. no.: P-ARAB) was determined by comparing amine phosphonate beads coated with the carbohydrate to control beads whose surface amines were acetylated. Sugar beet arabinan is a polymer containing the monosaccharides arabinose, galactose, rhamnose, and galacturonic acid. Neutral monosaccharides were quantified after hydrolysis of bead-bound polysaccharide. Arabinose liberated during acid hydrolysis of sugar beet arabinan-coated beads was used as a marker of arabinan degradation. Comparison of input beads to beads passed through germ-free animals demonstrates that sugar beet arabinan is not digested by host enzymes during passage through a mouse (FIG. 17). However, beads gavaged into colonized mice exhibited reduced levels of arabinan remaining on the surface, and the levels of degradation changed as a function of mouse diet. The microbiota of mice fed a diet high in saturated fat and low in fruits and vegetables (HiSF-LoFV) or mice fed a HiSF-LoFV diet supplemented with 100 mg/mouse/day sugar beet arabinan degraded a significant amount of sugar beet arbainan when compared to input beads that were not gavaged into mice colonized with a defined 14-member consortium composed of human gut microbiota that had been cultured and their genomes sequenced (Table 12) (Ridaura et al., 2013; Wu et al., 2015). Additionally, colonized mice fed HiSF-LoFV diet supplemented sugar beet arabinan showed increased degradation capacity as compared to colonized mice fed the unsupplemented HiSF-LoFV diet (p=0.086; pairwise Welch's t-test). These results demonstrate that 1) the defined model human microbiota was required for sugar beet arabinan degradation and 2) dietary supplementation with sugar beet arabinan changed the functional capacity of the microbiota to degrade this glycan.

TABLE 12
Bacterial strains comprising the model
defined human gut community.

| Bacteria | Strain | Citation |
|---|---|---|
| Bacteroides ovatus | ATCC 8483 | (Wu et al., 2015) |
| | INSeq | |
| Bacteroides cellulosilyticus | WH2 INSeq | (Wu et al., 2015) |
| Bacteroides thetaiotaomicron | ATCC 7330 | (Wu et al., 2015) |
| | INSeq | |
| Bacteroides thetaiotaomicron | VPI-5482 | (Wu et al., 2015) |
| | INSeq | |
| Bacteroides vulgatus | ATCC 8482 | (Wu et al., 2015) |
| | INSeq | |
| Bacteroides caccae | TSDC17.2 | (Ridaura et al., 2013) |
| Bacteroides finegoldii | TSDC17.2 | (Ridaura et al., 2013) |
| Bacteroides massiliensis | TSDC17.2 | (Ridaura et al., 2013) |
| Collinsella aerofaciens | TSDC17.2 | (Ridaura et al., 2013) |
| Escherichia coli | TSDC17.2 | (Ridaura et al., 2013) |
| Odoribacter splanchnicus | TSDC17.2 | (Ridaura et al., 2013) |
| Parabacteroides distasonis | TSDC17.2 | (Ridaura et al., 2013) |
| Ruminococcaceae sp. | TSDC17.2 | (Ridaura et al., 2013) |
| Subdoligranulum variabile | TSDC17.2 | (Ridaura et al., 2013) |

Further details are provided below for the materials and methods used in the above experiments.

Synthesis of amine phosphonate beads: To a solution of microscopic (10 μm) paramagnetic silica beads (Millipore Sigma; Cat no: LSKMAGN01) in water, equal molar amounts of (3-am inopropyl)triethoxysilane (ATPS) (Sigma Aldrich) and 3-(trihydroxysilyl)propyl methylphosphonate (THPMP) (Sigma Aldrich) were added (Bagwe et al., 2006; Soto-Cantu et al., 2012). The reaction was allowed to proceed for 5 hours at 50° C. with shaking. The reaction was terminated with repeated washing of beads with water using a magnet.

Zeta potential measurement: Zeta potential was measured to track modification of the bead surface. Zeta potential measurements were obtained on a Malvern ZEN3600 using disposable Malvern zeta potential cuvettes. Measurements were obtained with the default settings of the instrument, using the refractive index of SiO₂ as the material, and water as the dispersant. Beads were resuspended to a concentration of 5×10⁵/mL in 10 mM (4-(2-hydroxyethyl)-1-piperazineethanesulfonic acid) (HEPES; pH 7.2) and analyzed in triplicate. Zeta potential of starting beads and beads monofunctionalized with ATPS or THPMP were used as standards.

Fluorophore labeling of amine phosphonate beads: Fluorophores were covalently bound to the bead surface to facilitate the multiplexed analysis of multiple bead types within a single animal. N-Hydroxysuccinimide ester (NHS)-activated fluorophores were dissolved in dimethyl sulfoxide (DMSO) at 1 mM. Resuspended fluorophore was diluted into a solution of 20 mM HEPES (pH 7.2) and 50 mM NaCl to a final concentration of 100 nM and incubated with amine phosphonate beads for 50 minutes at 22° C. Beads were washed repeatedly with water to terminate the reaction. The extent of fluorophore labeling was assessed on each bead type using flow cytometry. The concentration of fluorophore used was the lowest at which the bead populations could be reliably and easily distinguished via flow cytometry. Fluorophores and their sources: Alexa Fluor 488 NHS ester (Life Technologies; cat. no.: A20000), Promofluor 415 NHS ester (PromoKine; cat. no.: PK-PF415-1-01), Promofluor 633P NHS ester (PromoKine; cat. no.: PK-PF633P-1-01), and Promofluor 510-LSS NHS ester (PromoKine; cat. no.: PK-PF510LSS-1-01).

Amine phosphonate bead acetylation: Acetylation of bead surface amines was used to confirm the specific linkage of both fluorophore and polysaccharides to the bead surface. Acetylated beads were also used as an empty bead control when gavaged into mice. Bead surface amines were acetylated using acetic anhydride under anhydrous conditions. Amine phosphonate beads were washed repeatedly with multiple solvents with the goal of resuspending the beads in anhydrous methanol; beads were washed in water, then methanol, then anhydrous methanol. Pyridine (0.5 volume equivalents) was then added as a base followed by acetic anhydride (0.5 volume equivalents). The reaction was allowed to proceed for 3 hours at 22° C. and then quenched with repeated washing with water. The described acetylation conditions had no effect on the fluorescence of any of the four fluorophores tested.

Polysaccharide conjugation to amine phosphonate beads: Polysaccharides were dissolved at 3-10 mg/mL in 50 mM HEPES (pH 8) with heat and sonication. To a solution of polysaccharide (5 mg/mL) containing trimethylamine (0.5 equivalent), 1-cyano-4-dimethylaminopyridinium tetrafluoroborate (CDAP; Sigma Aldrich; 1 eq.) dissolved in DMSO was added. The optimal concentration of CDAP was found to be 0.2 mg of CDAP per mg of polysaccharide. The polysaccharide/CDAP solution was mixed for 5 minutes at 22° C. to allow for polysaccharide activation. Amine phosphonate beads resuspended in 50 mM HEPES (pH 8) were added to the activated polysaccharide solution and the reaction was allowed to proceed for 15 hours at 22° C. Any aggregated beads were resuspended with light sonication. The resulting isourea linkage between the bead and polysaccharide was reduced by addition of 2-picoline borane dissolved in DMSO (10% wt:wt) and incubation for 40 minutes at 40° C. The reaction was terminated with repeated washing in water and then 20 mM HEPES (pH 7.2) 50 mM NaCl. The described reaction conditions for polysaccharide conjugation or reduction had little or no effect on the fluorescence of any of the four fluorophores tested.

Bead counting: The absolute number of beads in a solution was determined with flow cytometry using CountBright Absolute Counting Beads (ThermoFisher Scientific; cat. no.: C36950) according to the manufacturer's suggested protocol.

Bead pooling and gavage into gnotobiotic mice: Pools of equal number of each bead type were prepared from fluorophore-labeled polysaccharide-coated amine phosphonate beads. The required number of a given bead type was sterilized with 70% ethanol for 10 minutes before washing with sterile water and 20 mM HEPES (pH 7.2), 50 mM NaCl, 0.01% bovine serum albumin, and 0.01% Tween-20. The different bead types were then pooled into a single mixture.

Pooled bead mixtures (10-15×10⁶ beads) were gavaged into gnotobiotic mice 4-6 hours prior to sacrifice. Beads were harvested from cecal contents using bead density and magnetism. Beads were sorted back into the original bead type using fluorescence-activated cell sorting (FACS; BD FACSAria III).

Quantitation of polysaccharide degradation: Polysaccharide degradation was determined by quantifying the amount of monosaccharide hydrolyzed from bead-bound polysaccharide after bead passage through a mouse. To do so, an equal number of beads were placed in crimp-top glass vials and hydrolyzed using 2 M trifluoroacetic acid for 2 hours at 95° C. The solution was reduced to dryness under reduced pressure. Liberated monosaccharides were reduced with methoxyamine (15 mg/mL in pyridine) for 15 hours at 37° C. Hydroxyl groups were silylated using N-Methyl-N-trimethylsilyltrifluoroacetamide (MSTFA)+1% 2,2,2-Trifluoro-N-methyl-N-(trimethylsilyl)-acetamide, chlorotrimethylsilane (TCMS) (ThermoFisher Scientific; Cat. no.: TS-48915) for 1 hour at 60° C. Samples were diluted with heptane and analyzed by GC-MS on Agilent 7890A gas chromatography system, coupled with a 5975C mass spectrometer detector (Agilent). Monosaccharide composition and quantitation were determined using chemical standards simultaneously derivatized.

### Example 10

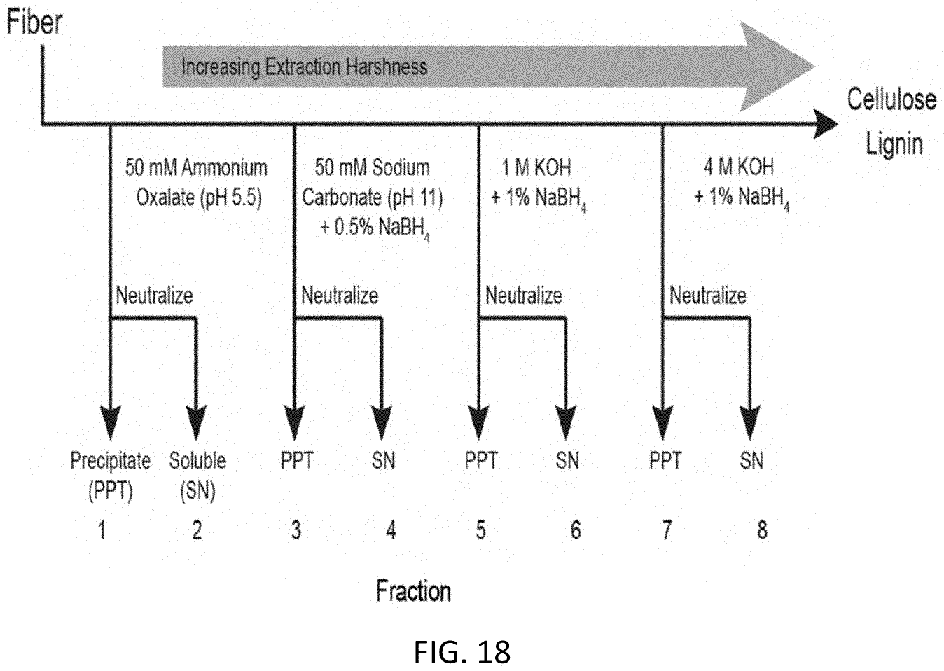

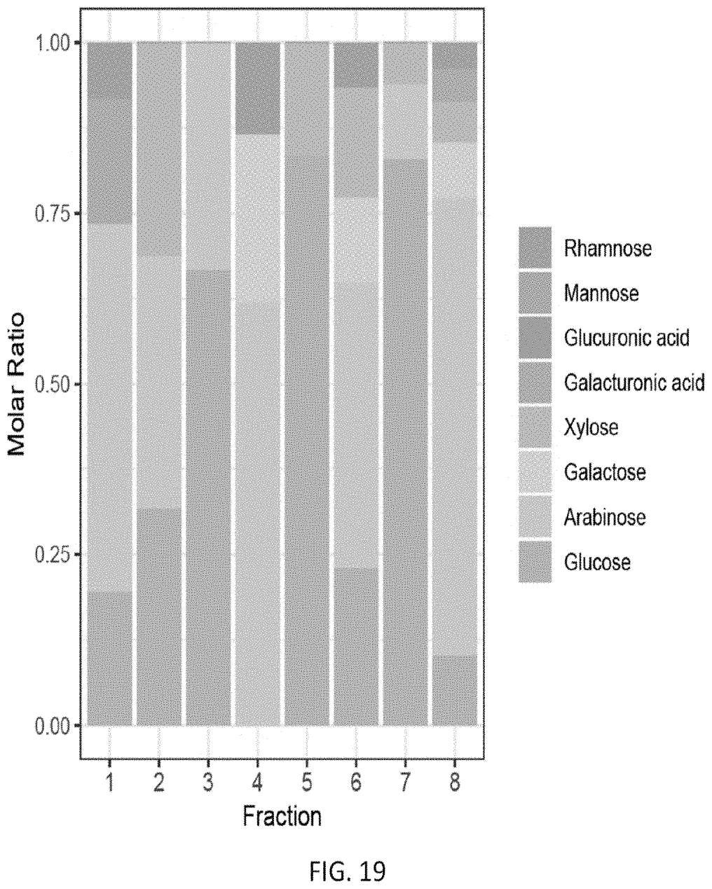

This example describes experiments to determine if there was a bioactive component of the pea fiber preparation used in Examples 2-6 that was responsible for increasing the representation of targeted Bacteroides represented in a model human gut community installed in gnotobiotic mice. The pea fiber preparation was subjected to extraction under increasingly harsh conditions with aqueous solutions to differentially solubilize constituents (Pattathil et al.) (FIG. 18). In total, 8 fractions were isolated and characterized for protein content (BCA assay), total carbohydrate content (phenol-sulfuric acid assay (Masuko et al.), and molecular size (high performance liquid chromatography-size exclusion chromatography with an evaporative light scattering detector). The monosaccharide composition of each fraction was determined (polysaccharide methanolysis followed by gas chromatography mass spectrometry (GC-MS; (Doco et al.)) (FIG. 19). Carbohydrate linkages were determined as partially methylated alditol acetates (PMAA) (Doares et al.).

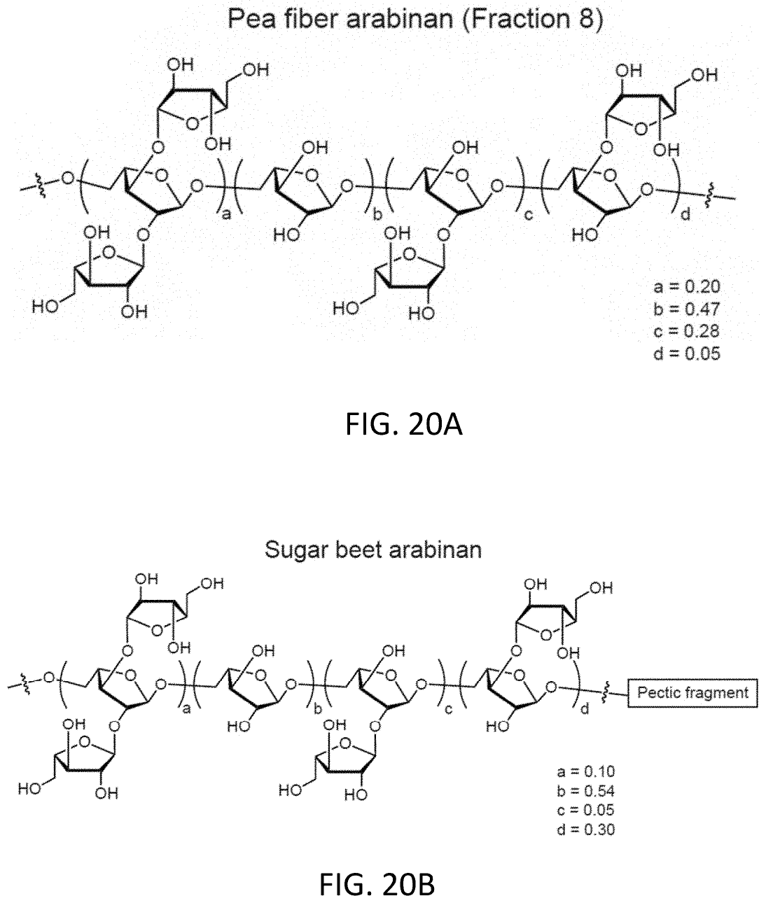

Fraction 8, obtained using the harshest conditions (4 M KOH for 24 hours at 22° C.) and containing high relative content of arabinose and galactose, was selected for further evaluation. Based on its monosaccharide composition and the results obtained from PMAA linkage analysis (Tables 13, 14), it appears that (i) fraction 8 is largely composed of arabinan that is predominately branched at the 2-, or doubly branched at the 2- and 3-positions of a linear al-5 L-arabinofuranose backbone (FIG. 20) and (ii) the arabinan is covalently attached to small pectic fragments containing galacturonic acid, galactose, and rhamnose. The structure of the pea fiber arabinan is more highly branched and sterically encumbered than the more commonly observed arabinan structure, exemplified by commercially available sugar beet arabinan which is branched almost exclusively at the 3-position (Megazyme; cat. no.: P-ARAB) (Tables 13, 14). In addition to arabinan, fraction 8 contains lesser amounts of two additional plant polysaccharides that are not covalently bound to the arabinan: a small amount of xylan (linear β1-4 xylose) and a small amount of starch (α1-4 glucose).

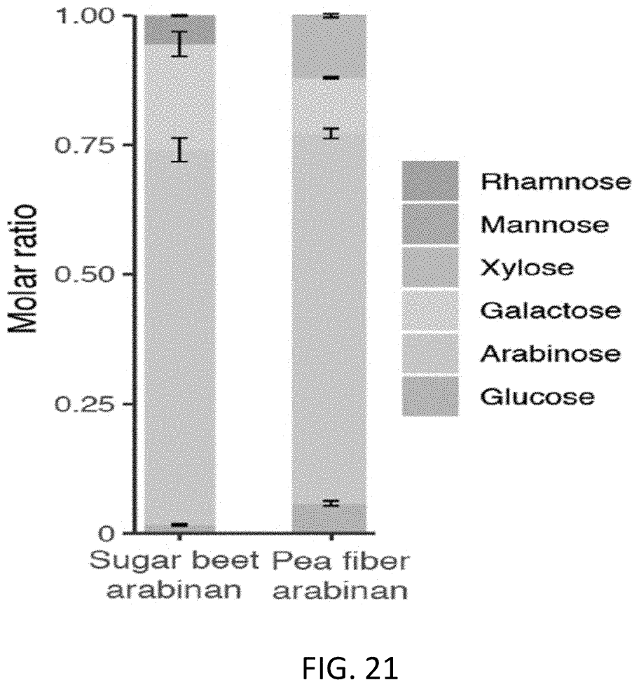

The method for Fraction 8 isolation was scaled up using a procedure similar to what was employed in the initial fractionation to supply sufficient quantities for studies in gnotobiotic mice (yield 22%±2% wt:wt) (FIG. 21). Briefly, 50 grams of the pea fiber preparation was first treated with 1 M KOH+0.5 wt. % sodium borohydride at room temperature for 24 hours to dissolve starch, proteins, free oligosaccharides and other smaller compounds. The mixtures were then centrifuged at 3,900 g for 20 minutes. The pellets were collected and resuspended in 4 M KOH+0.5 wt. % sodium borohydride and stirred at room temperature for 24 hours. The mixture was centrifuged at 3,900 g for 20 minutes again. The supernatant containing the targeted polysaccharides was then neutralized with 4 M acetic in cold bath. The extracted polysaccharides were then precipitated after adding ethanol to the mixture at the ratio of 3.75:1 and cooled down to −20° C. The precipitated polysaccharides were then collected by centrifuging the mixtures at 3,900 g at 4° C. for 20 minutes. The collected pellets were then crushed and washed in 80% ethanol at 4° C. to remove organics such as polyphenols. The latter step was repeated three times. The final pellets were then dried under dry nitrogen overnight to yield “Fraction 8”.

Next Fraction 8 (150 mg) was solubilized in 50 mM sodium malate (pH 6)+2 mM calcium chloride (30 mL) via incubation in a 95° C. water bath and sonication to yield a 5 mg/mL solution. To this, 3.5 mg of amyloglucoside (Megazyme; cat. no.: E-AMGFR) and 1.25 mg of alpha-amylase (Megazyme, cat. no.:E-PANAA) were added as 3 mg/mL stock solutions in 50 mM sodium malate (pH 6)+2 mM calcium chloride. Starch was digested via incubation at 37° C. for 4 hours. The digestion was terminated via enzyme denaturation by incubation at 90° C. for 30 min. The glucose product resulting from starch digestion was removed with extensive dialysis against ddH20 using 3.5 kDa molecular weight cut off Snakeskin dialysis tubing (ThermoFisher, cat. no: 88244). The sample was dried via lyophilization to yield enzymatically destarched Fraction 8. Monosaccharide analysis and glycosyl linkage analysis was performed as described above (Table 16 and Table 17). The enzymatically destarched Fraction 8 was then used in the following animal experiment.

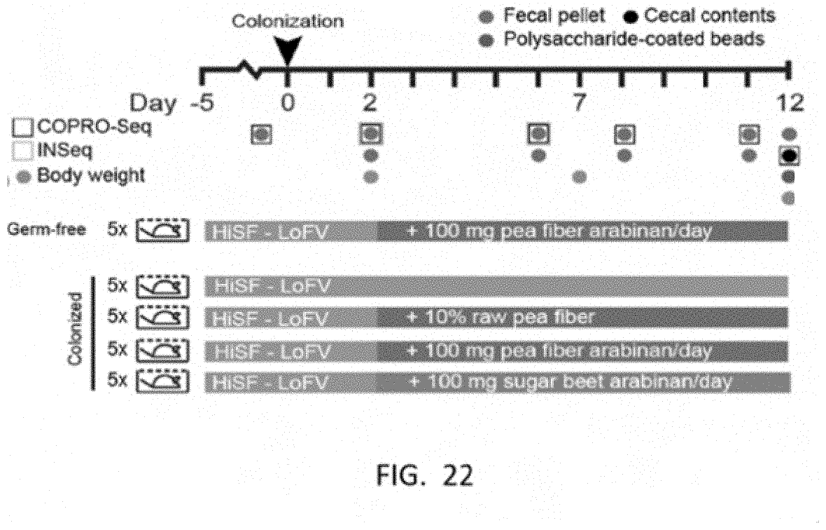

Four groups of adult C57BL/6J male mice fed the HiSF-LoFV diet were colonized with a defined community comprising 14 cultured, sequenced human gut bacterial strains (Ridaura et al.) (n=5 mice/arm; Table 15, FIG. 22). Two days after colonization, mice in three experimental groups were switched to the HiSF-LoFV diet supplemented with (i) 10% (wt:wt) the pea fiber preparation (calculated consumption 16.6 g/kg mouse weight/day), (ii) 100 mg/mouse/day enzymatically destarched Fraction 8 (3.3 g/kg/day), or (iii) 100 mg/mouse/day sugar beet arabinan (3.3 g/kg/day). A fourth control arm received the unsupplemented HiSF-LoFV diet.

Mice were given ad libitum access to the diets for 10 days at which point all animals were gavaged with polysaccharide-coated paramagnetic fluorescent beads. Animals were sacrificed 4 hours after gavage of the beads. Bacterial community composition was assessed via short read shotgun sequencing (COPRO-Seq) of DNA purified from serially-collected fecal samples and from cecal contents harvested at the conclusion of the experiment (McNulty et al.).

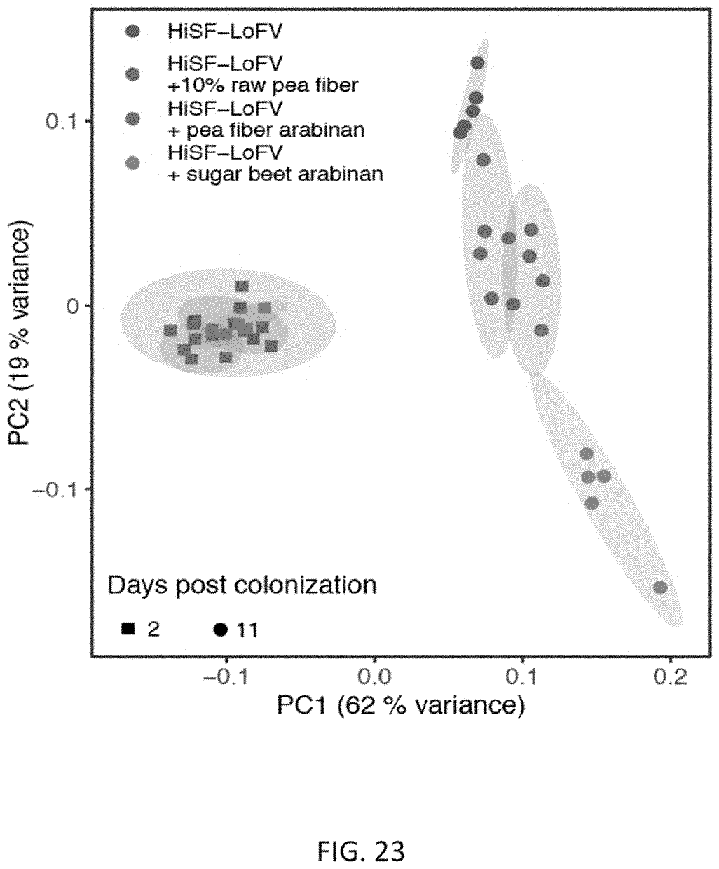

Principal components analysis of the relative abundances of community members in fecal samples collected on day 11 post-colonization revealed that all 3 experimental diets produced microbial community configurations that were distinct from those in mice consuming the control unsupplemented HiSF-LoFV diet (FIG. 23). Of note the microbial communities of mice supplemented with enzymatically destarched Fraction 8 were compositionally similar to that of mice whose diets were supplemented with the pea fiber preparation from which it was derived, but distinct from those consuming the sugar beet arabinan-supplemented HiSF-LoFV diet.

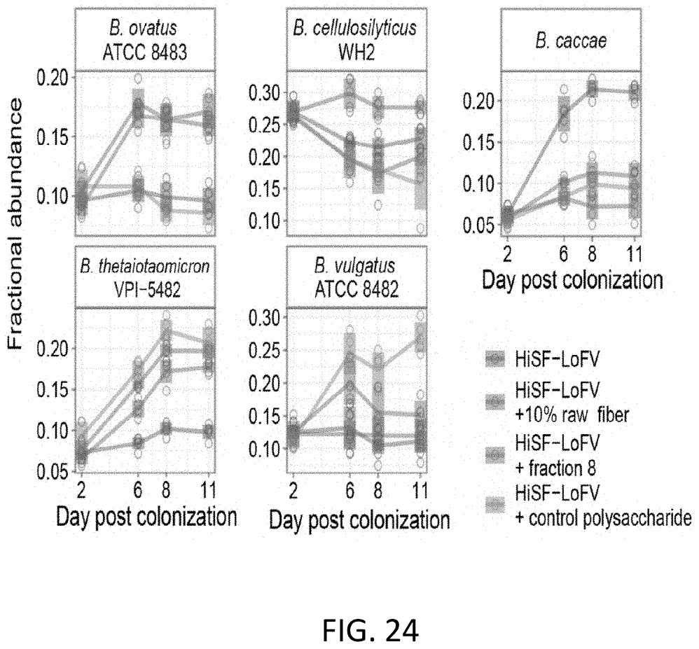

A time series analysis of the effects of the different glycans on the representation of community members in the fecal microbiota of mice belonging to the four treatment groups is presented in FIG. 24. Supplementaion with both enzymatically destarched Fraction 8 and the pea fiber preparation enhanced the fitness (relative abundance) of B. ovatus ATCC 8483 and B. thetaiotaomicron VPI-5482 compared to the unsupplemented HiSF-LoFV diet. In general, the responses of all Bacteroides to the pea fiber preparation and the enzymatically destarched Fraction 8 were similar (as judged by their relative abundances), the one exception being B. cellulosilyticus WH2, which achieved a higher representation in the community in the presence of the pea fiber preparation. In contrast, sugar beet arabinan differed from both the pea fiber preparation and the enzymatically destarched Fraction 8 in increasing the fractional abundance of B. vulgatus ATCC 8482 while having no significant effect on B. ovatus. Collectively, these results reveal that the enzymatically destarched Fraction 8 is able to recapitulate the majority of the effects on community composition of the pea fiber preparation from which it was derived, and also highlight the structural specificity of responses by different Bacteroides species to arabinan prepared from different plant sources.

We next sought to quantify how the in vivo degradative capacity of each individual mouse's microbiota changed with dietary fiber supplementation. To do so, we employed microscopic paramagnetic silica beads (average diameter=10 μm) with covalently bound glycans from the enzymatically destarched Fraction 8 or with purified sugar beet arabinan. Each bead type could be distinguished based on its distinct covalently linked fluorophore. Empty control beads contained no bound glycan. Beads were pooled and gavaged into mice colonized with the defined community and fed either the unsupplemented HiSF-LoFV, or the HiSF-LoFV supplemented with the pea fiber preparation, the enzymatically destarched Fraction 8 or the purified sugar beet arabinan. A separate group of animals that were maintained as germ-free fed the enzymatically destarched Fraction 8 supplemented HiSF-LoFV served as controls (n=5 m ice/treatment group)

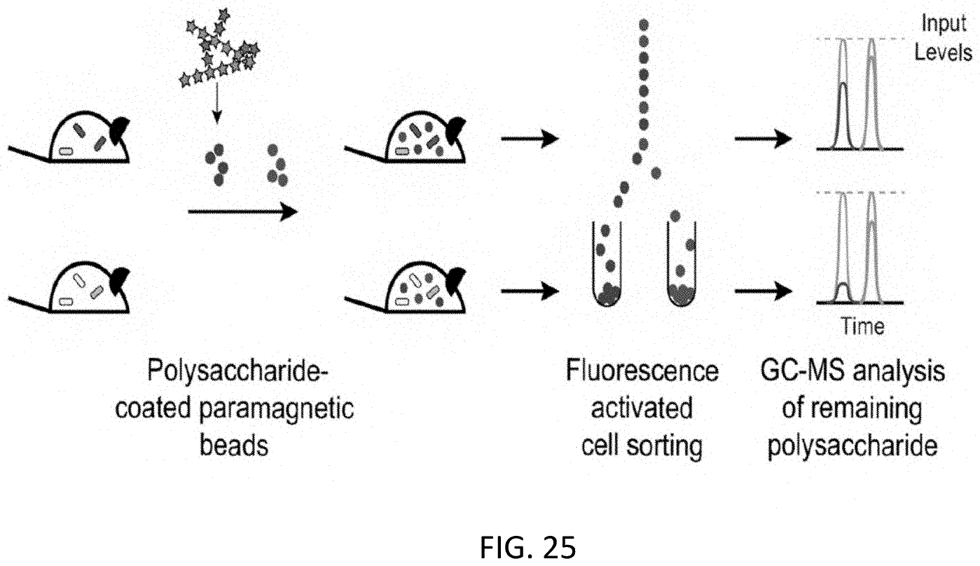

Animals from all groups were euthanized 4 hours after gavage of the bead mixture. Beads were then separated from cecal contents based on their density and magnetism, and each bead type was purified using fluorescence activated cell sorting (FACS) (FIG. 25). To compare the in vivo degradative capacities of each diet-exposed microbiota, recovered sorted beads were subjected to acid hydrolysis to release all residual bead-bound polysaccharide as free monosaccharides which were then quantified using GC-MS.

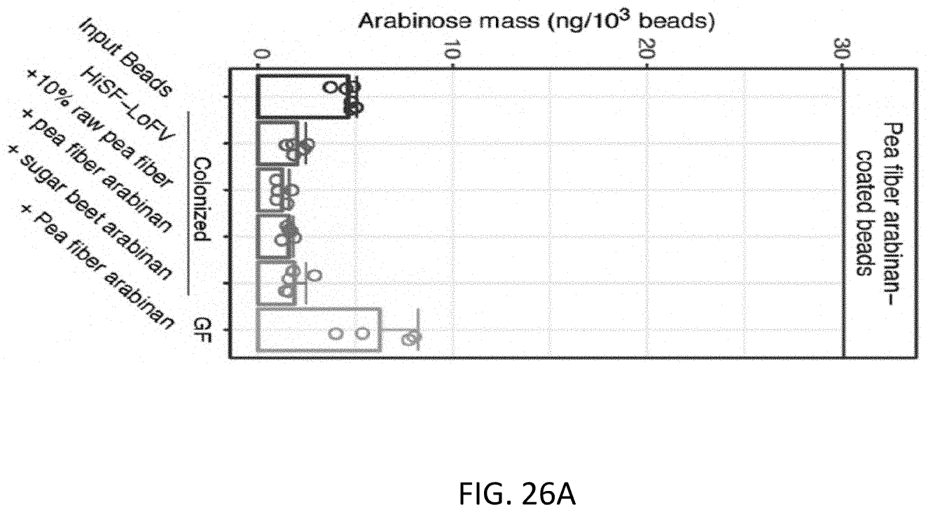

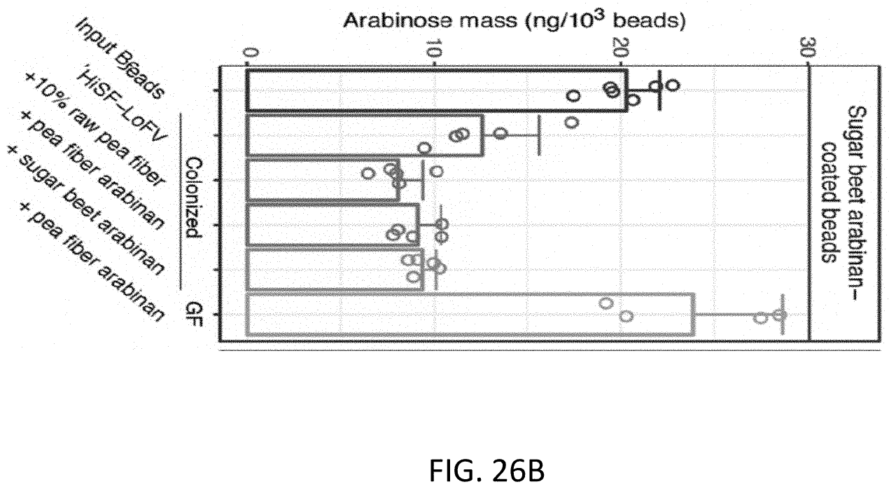

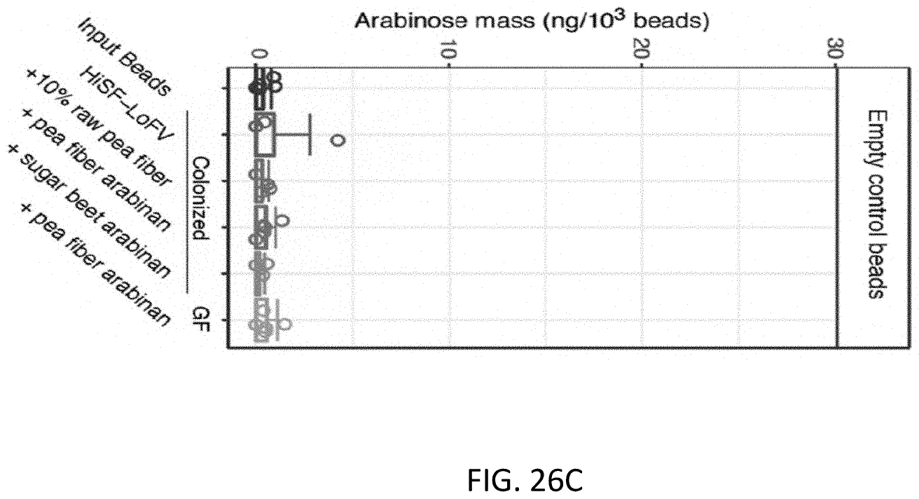

Comparison of germ-free controls to animals containing the defined consortium of human gut bacteria established that removal of arabinan from the different bead types was colonization-dependent. Moreover, no arabinose was detected in the empty beads that were administered to germ-free or colonized animals (FIG. 26). When colonized mice were fed the HiSF-LoFV diet supplemented with the pea fiber preparation, arabinose removal from beads with bound Fraction 8 glycans or sugar beet arabinan was significantly (p=0.018 and 0.025, respectively; unpaired t-test) enhanced compared to mice consuming the unsupplemented diet (FIG. 26). These results indicate that the pea fiber preparation has the capacity to change the functional configuration of the defined community to a state of enhanced capacity to process arabinan-containing polysaccharides. Mice fed the HiSF-LoFV diet supplemented with either enzymatically destarched Fraction 8 or sugar beet arabinan demonstrated a trend toward enhanced arabinose removal in both bead contexts compared to that in observed in mice fed the unsupplemented HiSF-LoFV diet (FIG. 26). These results might suggest that the purified (‘free’) forms of arabinan prepared from pea fiber (fraction 8), or sugar beet arabinan, compete with bead-bound arabinan for degradation/consumption by members of the community more effectively than the structurally bound, compositionally more complex pea fiber preparation, i.e., this more complex form requires additional processing by CAZymes before they are available to arabinan consumers represented in the model human gut microbiota.

TABLE 13
Percent fractional abundance of each detected linkage
in the purified sugar beet and fraction 8 preparations.

| | % Fractiona abundance | |
|---|---|---|
| | Sugar beet | |
| Residue | arabinan | Fraction 8 |
| Terminal Rhamnopyranosyl residue (t-Rha) | | 0.2 |
| Terminal Arabinofuranosyl residue (t-Ara(f)) | 21.3 | 20.5 |
| Terminal Fucopyranosyl residue (t-Fuc) | — | 0.7 |
| Terminal Arabinopyranosyl residue (t-Ara) | — | — |
| Terminal Xylopyranosyl residue (t-Xyl) | — | 3.8 |
| 2 linked Rhamnopyranosyl residue (2-Rha) | 0.8 | 0.2 |
| 2 linked Arabinofuranosyl residue (2-Ara(f)) | 0.6 | 0.3 |
| Terminal Glucuronic Acid residue (t-Glc A) | 0.7 | |
| Terminal Glucopyranosyl residue (t-Glc) | — | 0.8 |
| 3 linked Arabinofuranosyl residue (3-Ara(f)) | 0.7 | 0.1 |
| Terminal Galactopyranosyl residue (t-Gal) | 2.9 | 2.9 |
| 4 linked Arabinopyranosyl residue or | 29.3 | 20.7 |
| 5 linked Arabinofuranosyl residue | | |
| (4-Ara(p) or 5-Ara(f)) | | |
| 4 linked Xylopyranosyl residue (4-Xyl) | — | 3.8 |
| 2 linked Xylopyranosyl residue (2-Xyl) | — | 1.5 |
| 2,4 linked Rhamnopyranosyl residue (2,4-Rha) | 1.7 | 1.0 |
| 2 linked Glucopyranosyl residue (2-Glc) | 0.3 | |
| 3 linked Galactopyranosyl residue (3-Gal) | 1.5 | 1.5 |
| 2 linked Galactopyranosyl residue (2-Gal) | — | 0.7 |
| 3,4 linked Arabinopyranosyl residue or 3,5 | 24.5 | 2.3 |
| linked Arabinofuranosyl residue (3,4-Ara(p) | | |
| or 3,5-Ara(f)) | | |
| 4 linked Galactopyranosyl residue (4-Gal) | 6.0 | 3.1 |
| 4 linked Galacturonic Acid residue (4-Gal A) | 0.4 | |
| 4 linked Glucopyranosyl residue (4-Glc) | — | 19.1^{a} |
| 6 linked Galactopyranosyl residue (6-Gal) | 1.5 | — |
| 2,4 linked Arabinopyranosyl residue or 2,5 | 1.7 | 6.0^{a} |
| linked Arabinofuranosyl residue (2,4-Ara(p) | | |
| or 2,5-Ara(f)) | | |
| 2,3,4 linked Arabinopyranosyl residue or | 4.2 | 7.8 |
| 2,3,5 linked Arabinofuranosyl residue (2,3,4- | | |
| Ara(p) or 2,3,5-Ara(f)) | | |
| 3,4 linked Glucopyranosyl residue (3,4-Glc) | — | — |
| 2,4 linked Glucopyranosyl residue (2,4-Glc) | — | — |
| 3,6 linked Galactopyranosyl residue (3,6-Gal) | 1.7 | |
| 4,6 linked Glucopyranosyl residue (4,6-Glc) | — | 3.1 |

^{a}These 2 peaks overlapped; percentages were estimated based on MS fragmentation

TABLE 14
Percent fractional abundance of each detected arabinose
linkage relative to the total arabinose linkages
in purified sugar beet and Fraction 8.

| | % Fractional abundance | |
|---|---|---|
| Residue | Sugar beet arabinan | Fraction 8 |
| Terminal Arabinofuranosyl | 25.9 | 35.5 |
| residue (t-Ara(f)) | | |
| 2 linked Arabinofuranosyl | 0.7 | 0.5 |
| residue (2-Ara(f)) | | |
| 3 linked Arabinofuranosyl | 0.9 | 0.2 |
| residue (3-Ara(f)) | | |
| 4 linked Arabinopyranosyl | 35.6 | 25.9 |
| residue or 5 linked | | |
| Arabinofuranosyl residue (4-Ara(p) | | |
| or 5-Ara(f)) | | |
| 3,4 linked Arabinopyranosyl | 29.8 | 4.0 |
| residue or 3,5 linked | | |
| Arabinofuranosyl residue (3,4- | | |
| Ara(p) or 3,5-Ara(f)) | | |
| 2,4 linked Arabinopyranosyl | 2.1 | 10.4^{a} |
| residue or 2,5 linked | | |
| Arabinofuranosyl residue (2,4- | | |
| Ara(p) or 2,5-Ara(f)) | | |
| 2,3,4 linked Arabinopyranosyl | 5.1 | 13.5 |
| residue or 2,3,5 linked | | |
| Arabinofuranosyl residue | | |
| (2,3,4-Ara(p) or 2,3,5-Ara(f)) | | |

^{a}Peak overlapped with another peak; percentage estimated based on MS fragmentation

TABLE 15
Bacterial strains comprising the model
defined human gut community.

| Bacteria | Strain | Citation |
|---|---|---|
| Bacteroides ovatus | ATCC 8483 INSeq | Ridaura et al. |
| Bacteroides cellulosilyticus | WH2 INSeq | Ridaura et al. |
| Bacteroides thetaiotaomicron | ATCC 7330 INSeq | Ridaura et al. |
| Bacteroides thetaiotaomicron | VPI-5482 INSeq | Ridaura et al. |
| Bacteroides vulgatus | ATCC 8482 INSeq | Ridaura et al. |
| Bacteroides caccae | TSDC17.2 | Wu et al. |
| Bacteroides finegoldii | TSDC17.2 | Wu et al. |
| Bacteroides massiliensis | TSDC17.2 | Wu et al. |
| Collinsella aerofaciens | TSDC17.2 | Wu et al. |

TABLE 16
Percent fractional abundance of linkages in
the enzymatically destarched Fraction 8

| Glycosyl linkage | % Fractional abundance |
|---|---|
| t-Rha(p) | 0.15% |
| t-Ara(f) | 19.37% |
| t-Fuc(p) | 0.46% |
| t-Ara(p) | 0.13% |
| t-Xyl(P) | 2.66% |
| 2-Rha(p) | 1.30% |
| t-Man(p) | 0.36% |
| 3-Rha(p) | 0.10% |
| t-Glc(p) | 0.16% |
| 3-Ara(f) | 0.34% |
| t-Gal(p) | 2.93% |
| 4-Ara(p)/5-Ara(f) | 21.78% |
| 3′-Api(f) | 0.17% |
| 4-Xyl(p) | 6.52% |
| 2,3-Rha(p) | 0.00% |
| 2,4-Rha(p) | 2.71% |
| 2,3,4-Rha(p) | 0.34% |
| 3-Gal(p) | 1.91% |
| 2-Gal(p) | 0.66% |
| 3,4-Ara(p)/3,5-Ara(f) | 2.21% |
| 2,4-Ara(p)/2,5Ara(f) | 13.07% |
| 4-Gal(p) | 7.22% |
| 2,3,4-Ara(p)/2,3,5-Ara(f) | 9.44% |
| 4-Glc(p) | 0.07% |
| 3,4-Xyl(P) | 0.91% |
| 2,4-Glc(p) | 0.34% |
| 2,3,4-Xyl(p) | 0.22% |
| 3,6-Man(p) | 0.00% |
| 2,6-Man(p) | 0.03% |
| 4,6-Glc(p) | 0.05% |
| 4,6-Gal(p) | 4.38% |
| 3,6-Gal(p) | 0.54% |
| 3,4,6-Gal(p) | 0.29% |
| 2,3,6-Gal(p) | 0.02% |
| | 100.84% |

TABLE 17
Fractional abundance of arabinose monosaccharides. Abundance
is relative to total arabinose content. Data was generated
from enzymatically destarched fraction #8 as partially
methylated alditol acetate via GC-MS analysis which was
supported by the Chemical Sciences, Geosciences and Biosciences
Division, Office of Basic Energy Sciences, U.S. Department
of Energy grant (DE-SC0015662) to DOE - Center for Plant
and Microbial Complex Carbohydrates at the Complex Carbohydrate
Research Center.

| | % fractional abundance of |
|---|---|
| | arabinose monosaccharides |
| Monosaccharide | (relative to total arabinose) |
| t-Ara(f) | 29.20% |
| t-Ara(p) | 0.20% |
| 3-Ara(f) | 0.51% |
| 4-Ara(p)/5-Ara(f) | 32.83% |
| 3,4-Ara(p)/3,5-Ara(f) | 3.33% |
| 2,4-Ara(p)/2,5Ara(f) | 19.70% |
| 2,3,4-Ara(p)/2,3,5-Ara(f) | 14.23% |
| | 100.00% |

### Example 11

Fiber preparations were evaluated in various product formats for a number of attributes relating to production (e.g., dough processability, etc.) and organoleptic qualities (e.g., taste, texture, etc.). An “acceptable” product (A) was determined to have suitable processability, taste and texture. An “unacceptable” product (U) was deficient in processability, taste and/or texture.

Table 18 summarizes the findings from tests of three fiber compositions. In each product format, the indicated fiber composition provided 3 g, 6 g, or 10 g of dietary fiber. The remaining ingredients contributed additional dietary fiber. The “Pea” composition consisted of 100 wt % pea fiber. The “2 Fiber” composition consisted of 33 wt % pea fiber, 36 wt % high molecular weight inulin, 11 wt % orange fiber, and 20 wt % barley fiber. Attributes of the various fiber preparations are provided in Table A, Table B, Table C1, Table E, and Table F1.

TABLE 18

| | Product Format | | | | | | | | |
|---|---|---|---|---|---|---|---|---|---|
| | 3 g Dietary Fiber* per serving | | | 6 g Dietary Fiber* per serving | | | 10 g Dietary Fiber* per serving | | |
| | Fiber Comp. | | | | | | | | |
| | Pea | 2 Fiber | 4 Fiber | Pea | 2 Fiber | 4 Fiber | Pea | 2 Fiber | 4 Fiber |
| Cracker | A | A | A | A | NT | NT | U | U | U |
| Cookie, | A | A | A | NT | NT | NT | U | U | U |
| Sweet Bites | | | | | | | | | |
| Bars | A | A | A | A | A | A | NT | NT | NT |
| Extruded | A | A | A | A | A | A | A | A | A |
| Extruded Filled | A | A | A | NT | NT | NT | U | U | U |

*Amount of Dietary Fiber in the product contributed by the Fiber Composition.
NT = not tested

Based on the above testing, additional work was done to further improve overall sensory attributes by optimizing the additional ingredients in a given product format. Table 19 contains several representative products.

TABLE 19

| | | | | | | % | | |
|---|---|---|---|---|---|---|---|---|
| | % | | | | | Total Dietary | Dietary Fiber from | mg |
| Product Type | Moisture | Ash | Protein | Fat | Carb. | Fiber | fiber composition | Sodium |
| Cracker | 1.6 | 2.0 | 7.4 | 19.5 | 69.5 | 14.9 | 9.3 | 425 |
| w/mushrooms | | | | | | | | |
| Cracker | 1.7 | 1.6 | 7.4 | 19.3 | 70.0 | 16.1 | 9.9 | 328 |
| w/inclusions | | | | | | | | |
| Cookie | 2.6 | 1.4 | 5.7 | 20.1 | 70.1 | 10.3 | 5.7 | 224 |
| w/yoghurt | | | | | | | | |
| Ginger cookie | 8.6 | 1.3 | 3.0 | 13.4 | 73.8 | 8.8 | 7.8 | 221 |
| Extruded pillow | 1.8 | 2.9 | 13.7 | 35.5 | 46.2 | 14.7 | 12.3 | 735 |
| w/almond filling | | | | | | | | |
| Extruded pillow | 2.9 | 1.7 | 3.3 | 13.4 | 78.6 | 29.8 | 28.8 | 512 |
| w/probiotic | | | | | | | | |

Protein, fat, ash, and moisture content were measured by methods established by Association of Official Analytical Chemists (AOAC) 2009.01, AOAC 920.123, AOAC 933.05, AOAC 935.42, and AOAC 926.08, respectively. Carbohydrate is calculated as (100 − (Protein + Fat + Ash + Moisture). Total dietary fiber was measured by AOAC method 2009.01.

### Introduction to Examples 12-17

Examples 12-17 describe and execute an approach for developing microbiome-directed foods (MDF) that reconfigure the gut community in ways that improve nutritional status. Gnotobiotic mice, colonized with microbiomes from nine obese adults, were fed a prototypical Western diet, high in saturated fats and low in fruits and vegetables (HiSF-LoFV) supplemented with different plant fiber preparations. Fiber-discriminating responses of bacterial taxa, carbohydrate-active enzyme genes (CAZymes) and metabolic pathways in the microbiome were identified using feature reduction methods. Snack food prototypes containing one, two or four fiber preparations were administered for 2-3-week-long periods to overweight or obese adults consuming a controlled HiSF-LoFV diet. Analyses of serially sampled microbiomes and ˜1300 plasma proteins identified fiber-specific changes in the representation of CAZymes that correlated with alterations in the proteome indicative of improved health status.

### Example 12. Effects of a HiSF-LoFV Diet in Gnotobiotic Mice

Three plant fiber preparations to be included in a fiber-supplemented HiSF-LoFV (high in saturated fats and low in fruits and vegetables) diet were selected based on their affordability, reliable sourcing, predicted/known sensory properties, and postulated feasibility for incorporation into food prototypes. Fibers isolated from the pea Pisum sativum, the vesicular pulp of the orange Citrus sinensis, and the bran of barley (Hordeum vulgare) all contain a diverse set of glycan constituents. Arabinan and galacturonan are the most abundant glycans in pea fiber as defined by monosaccharide composition (22.4% arabinose [Ara] and 13.9% galacturonic acid [GalA]), and detection of α-1,5-Ara and α-1,4-GalA linkages by permethylation analysis (Table 26). In gnotobiotic mice colonized with a 20-member consortium of gut bacterial strains cultured from a single Ln donor revealed that pea fiber induced a marked increase in the abundance of Bacteroides thetaiotaomicron (15). Forward genetic screens and high-resolution mass spectrometry analysis of their fecal meta-proteomes identified pea-fiber-dependent increases in the expression of genes that were important fitness factors; they encode members of glycoside hydrolase (GH) families GH51, GH43_4, and GH146, that cleave linear α-1,5-Ara linkages, and α-1,2- and α-1,3-Ara branching linkages (15). Orange fiber also contains arabinan and galacturonan, but in contrast to pea fiber, galacturonan dominates (13.9% Ara, 42.9% GalA) (Table 26). Orange fiber administration also resulted in a pronounced increase in the abundance of Bacteroides thetaiotaomicron (15). Barley bran contains 17% mixed-linkage β-glucans; arabinose and xylose (7.1% and 9.9%) are represented in arabinoxylans (linear β-1,4-linked xylose with terminal α-1,2- and α-1,3-linked arabinose substitutions) (Table 26). Barley bran was one of the most active fibers screened in our previous study in gnotobiotic mice, producing a 3% increase in the relative abundance of B. ovatus for every 1% w/w increase in the fiber (15). Forward genetic and proteomic analyses were not performed in mice consuming orange fiber- or barley bran-supplemented HiSF-LoFV diets.

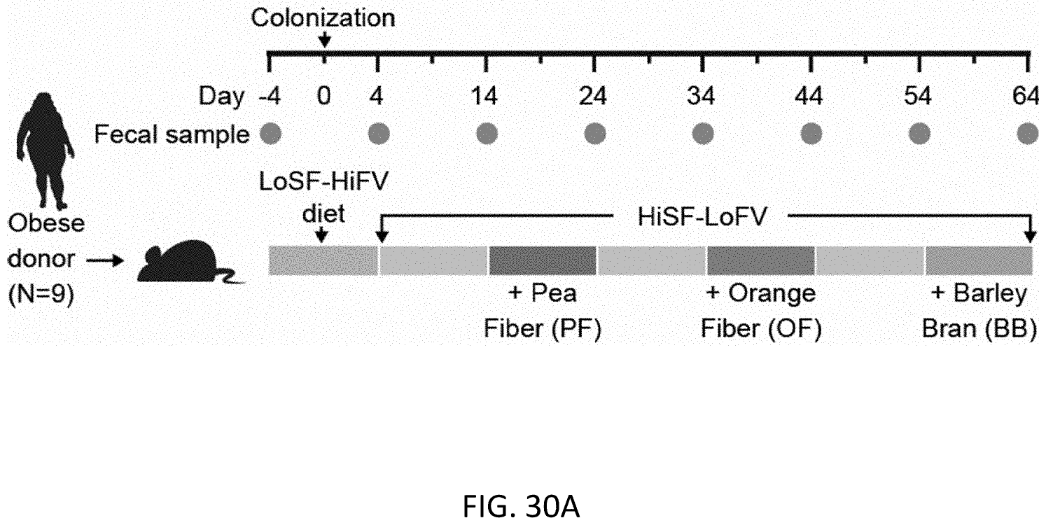

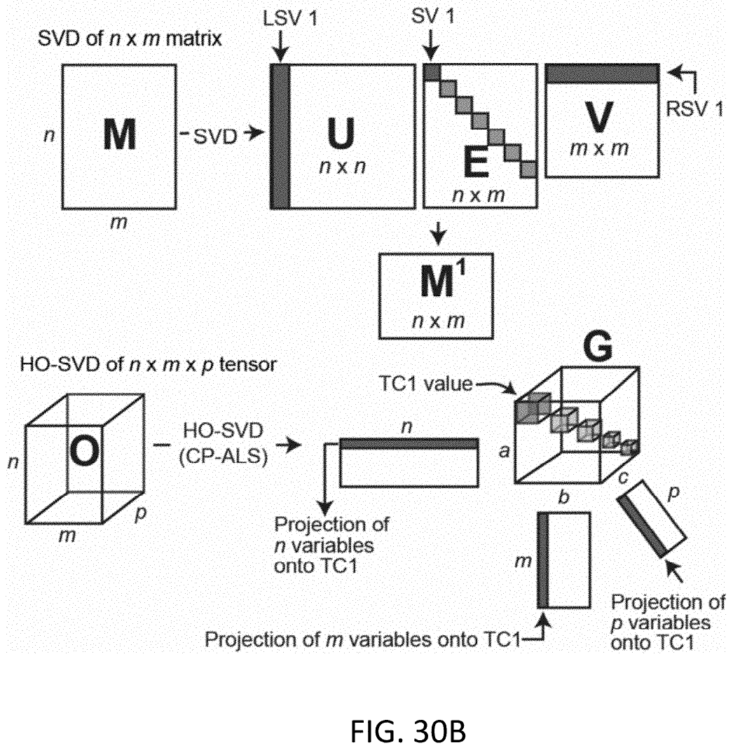

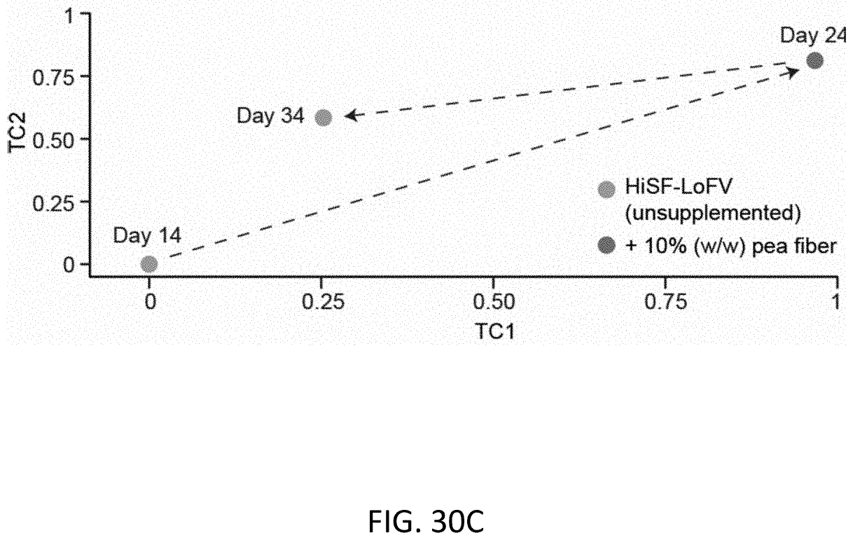

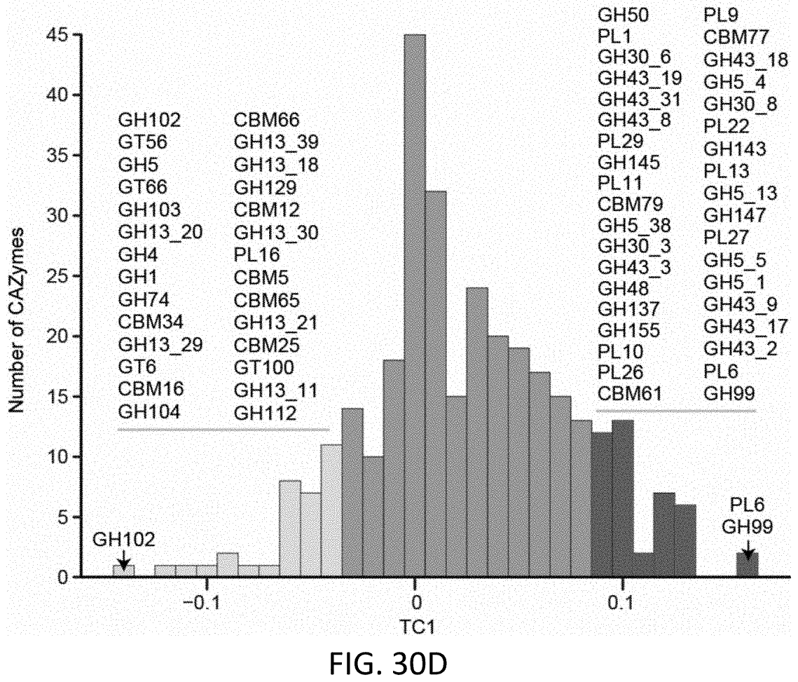

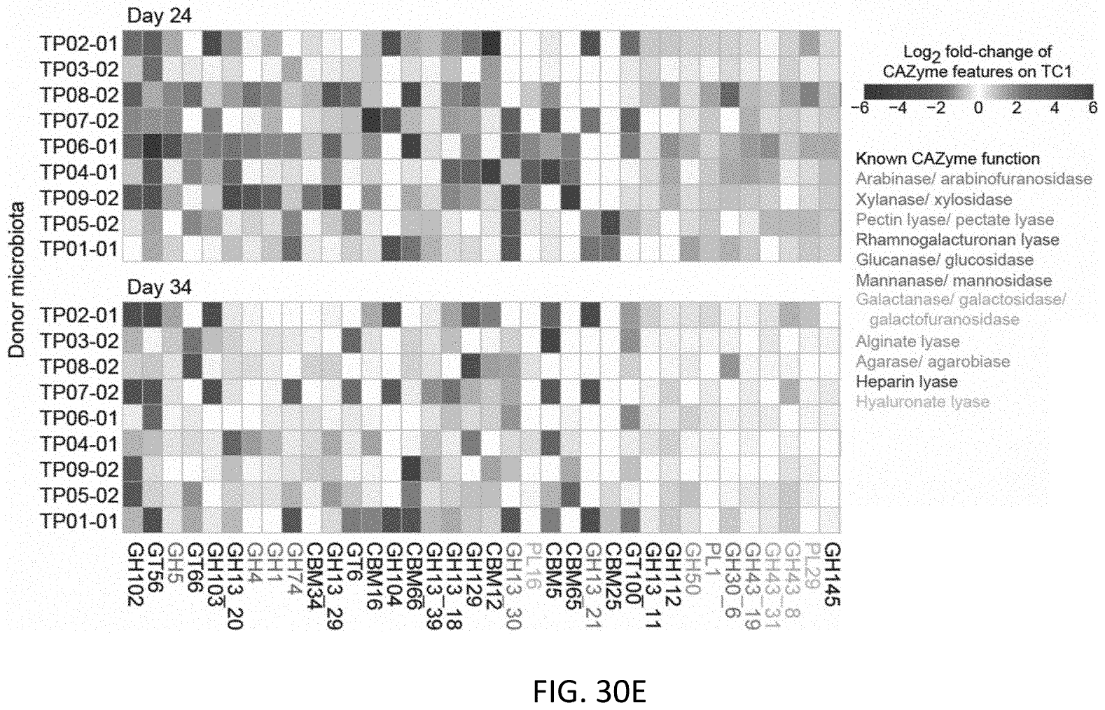

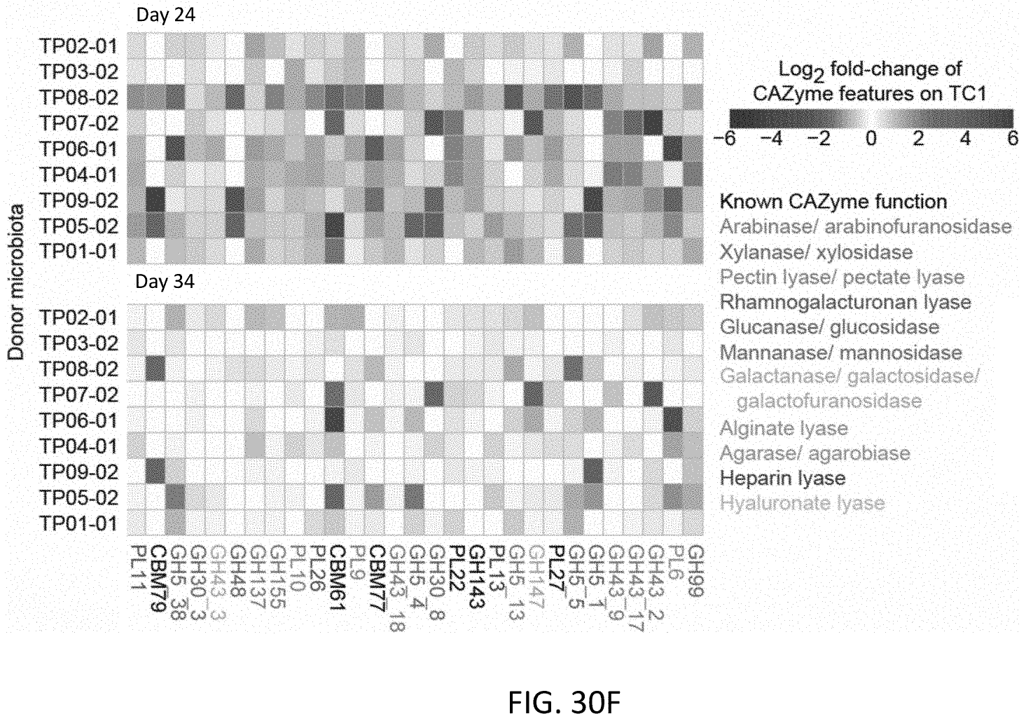

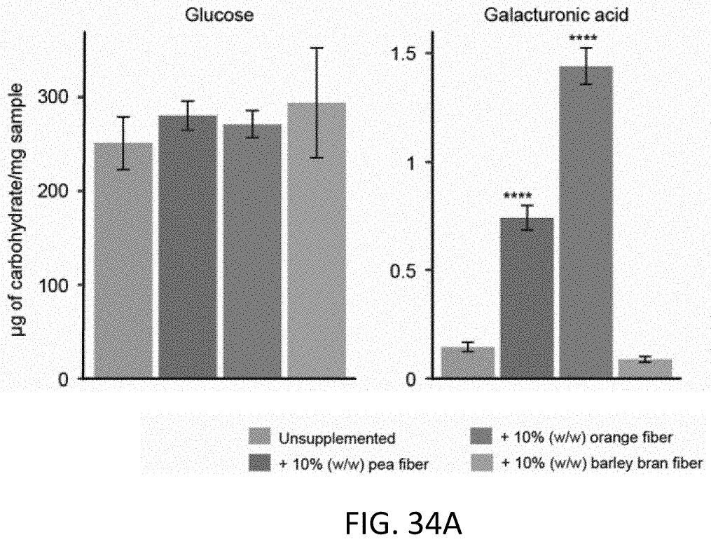

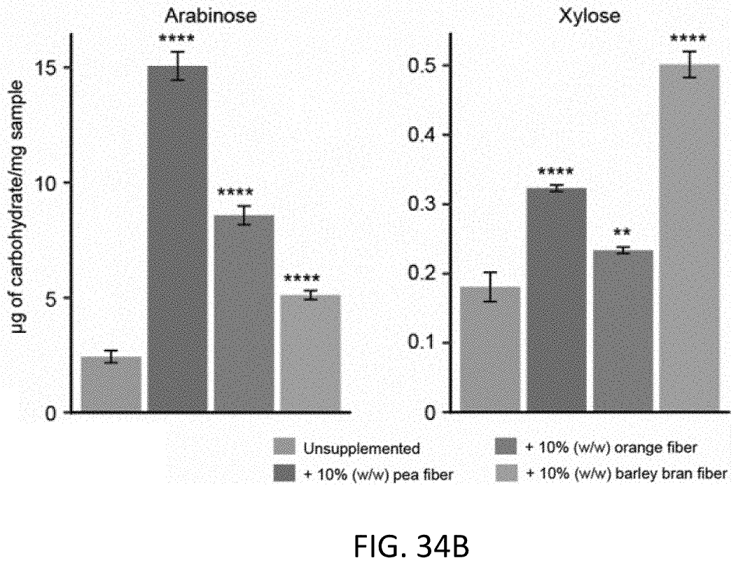

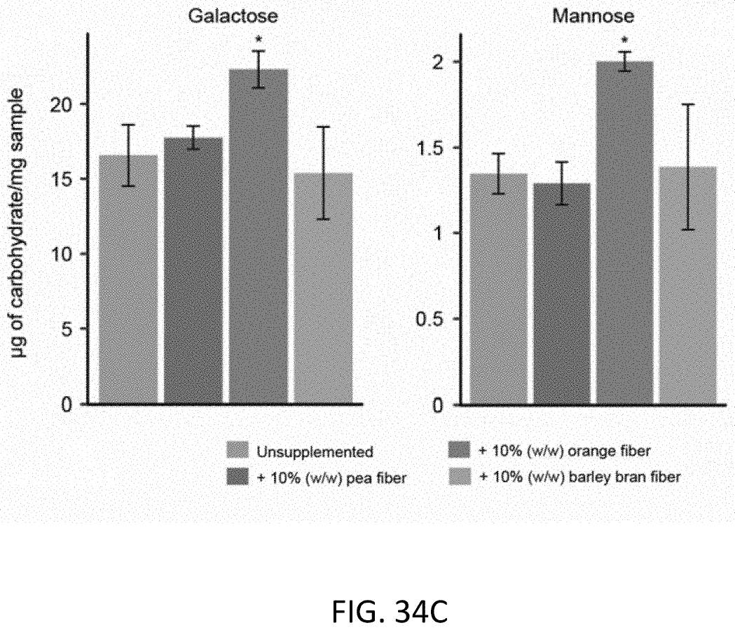

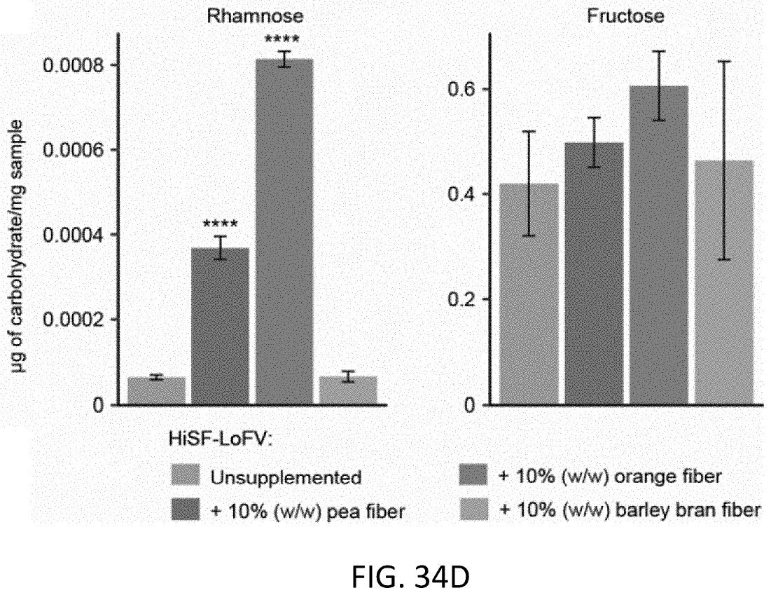

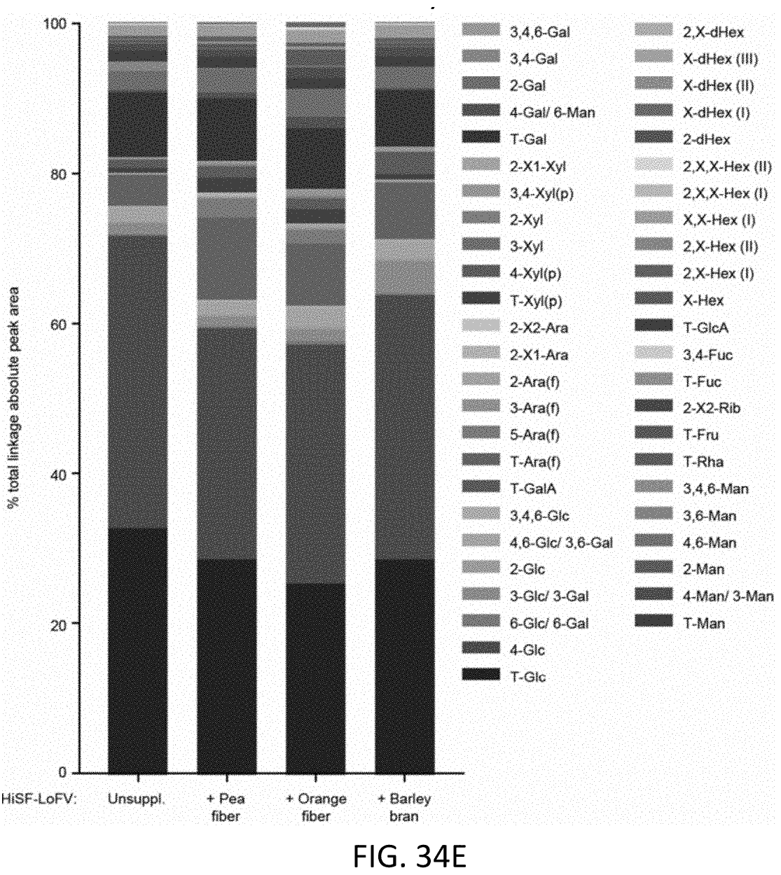

Nine groups of 12-to-16-week-old gnotobiotic mice were each colonized with a fecal sample obtained from one of nine 32-41-year-old women with obesity. Each mouse in each treatment group was subjected to the diet oscillation protocol summarized in FIG. 30A. The base HiSF-LoFV diet was supplemented with 10% (w/w) of one of the three types of food-grade fibers (see FIG. 34, Table 20, and Table 26). Mice consumed each fiber-supplemented HiSF-LoFV diet monotonously for 10 days. The unsupplemented HiSF-LoFV diet was given for 10 days between each period of fiber-supplemented diet consumption with fecal samples obtained on the day preceding and on the last day of each 10-day cycle. DNA was prepared from fecal samples, amplicons were generated by PCR of variable region 4 (V4) of bacterial 16S rDNA genes to identify bacterial taxa in the microbiota, and whole community DNA was subjected to shotgun sequencing to identify genes in the microbiome. A total of 381 taxa [amplicon sequence variants (ASVs)] were identified (threshold for inclusion in the analysis: present at a relative abundance of 0.1% in at least five samples collected from the 69 microbiota transplant recipients). Shotgun sequencing reads of fecal microbiomes were assembled and annotated. The annotation focused on (i) the representation of carbohydrate-active enzymes represented in the Carbohydrate-Active enZYmes (CAZy) database [includes glycoside hydrolases, polysaccharide lyases, carbohydrate-binding modules, and glycosyltransferases (16), and (ii) metabolic pathways involved in carbohydrate utilization and fermentation, biosynthesis of amino acids, and B-vitamins/cofactors (the latter play a critical role in myriad metabolic reactions). Metabolic pathway annotations were based on the RAST/SEED platform; this platform combines homology- and genome context-based evidence with known sets of enzymatic reactions and nutrient transporters to group genes into ‘microbial community (mc) subsystems’ (mcSEED subsystems) that capture and project variations in particular metabolic pathways/modules across thousands of microbial genomes (17-19).

TABLE 20

| | HiSF-LoFV diet | HiSF-LoFV diet + | HiSF-LoFV diet + | HiSF-LoFV diet + |
|---|---|---|---|---|
| | (unsupplemented) | 10% (w/w) pea | 10% (w/w) orange | 10% (w/w) barley |
| Nutritional composition | per 100 g | fiber per 100 g | fiber per 100 g | bran per 100 g |
| Total energy (kcal) | 469.0 | 422.1 | 422.1 | 422.1 |
| Fat (% kcal) | 39.0 | 39.0 | 39.0 | 39.0 |
| Saturated fat (% kcal) | 16.0 | 16.0 | 16.0 | 16.0 |
| Monounsaturated fat (% | 11.0 | 11.0 | 11.0 | 11.0 |
| kcal) | | | | |
| Polyunsaturated fat (% | 7.0 | 7.0 | 7.0 | 7.0 |
| kcal) | | | | |
| Cholesterol (mg/100 g) | 74.0 | 66.6 | 66.6 | 66.6 |
| Protein (% kcal) | 18.0 | 18.0 | 18.0 | 18.0 |
| Carbohydrate (% kcal) | 43.0 | 43.0 | 43.0 | 43.0 |
| Sugars (% kcal) | 22.0 | 22.0 | 22.0 | 22.0 |
| Total dietary fiber (g) | 3.1 | 13.1 | 13.1 | 13.1 |
| Pea fiber (g) | — | 10.0 | — | — |
| Orange fiber (coarse) (g) | — | — | 10.0 | — |
| Barley bran (g) | — | — | — | 10.0 |

| Nutritional composition | | | Orange fiber | |
|---|---|---|---|---|
| per fiber | — | Pea fiber | (coarse) | Barley bran |
| % Total dietary fiber | — | 67.2 | 68.5 | 46.0 |
| % Protein | — | 9.5 | 7.5 | 18.7 |
| % Fat | — | 0.9 | 2.2 | 4.1 |
| % Carbohydrate | — | 79.8 | 80.9 | 69.3 |
| % Moisture | — | 7.4 | 5.7 | 5.7 |
| % Ash | — | 2.5 | 2.0 | 2.0 |

### Example 13. Effects of Dietary Fiber on Microbial Community Configuration

Singular Value Decomposition (SVD) is a method used for dimension reduction where substantial compression of information is often sought (FIG. 30B). However, a substantial limitation of SVD is that it can only be used on datasets comprising two feature types (e.g., samples as rows, microbial genes or taxa as columns). In cases where a third feature type is used (temporal data or different conditions) a generalization of SVD to higher dimensions, termed ‘Higher-Order Singular Value Decomposition’ (HO-SVD), can be used. Briefly, this technique relates variation among all dimensions included in the data and can be extended to an arbitrary number of dimensions (FIG. 30B and Methods for details).

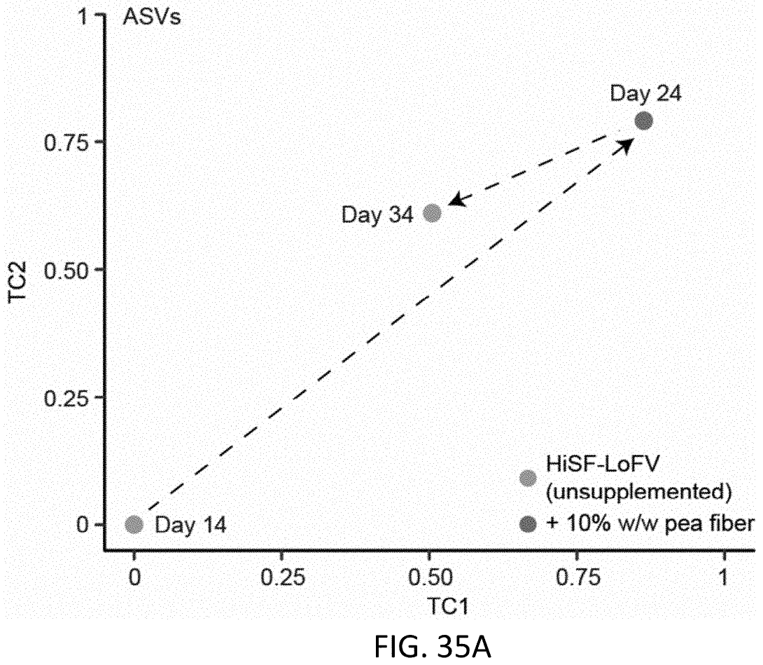

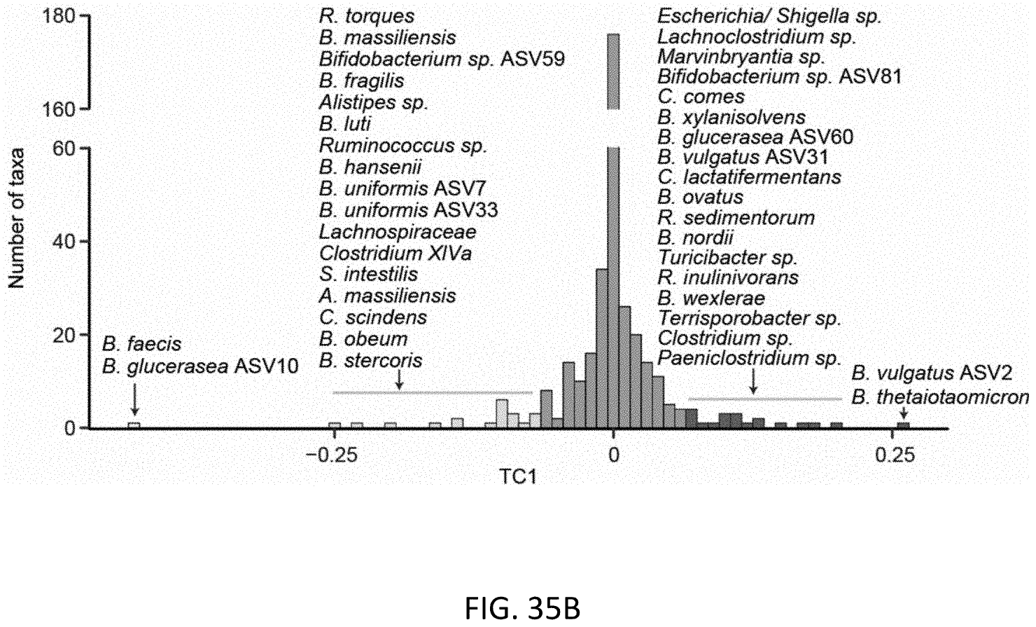

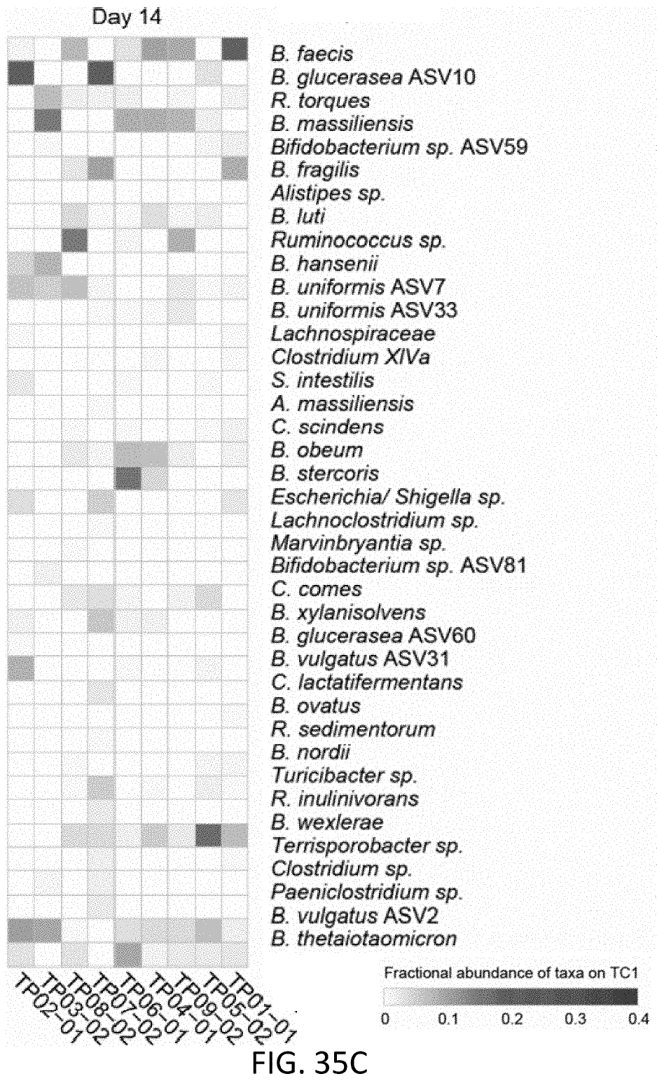

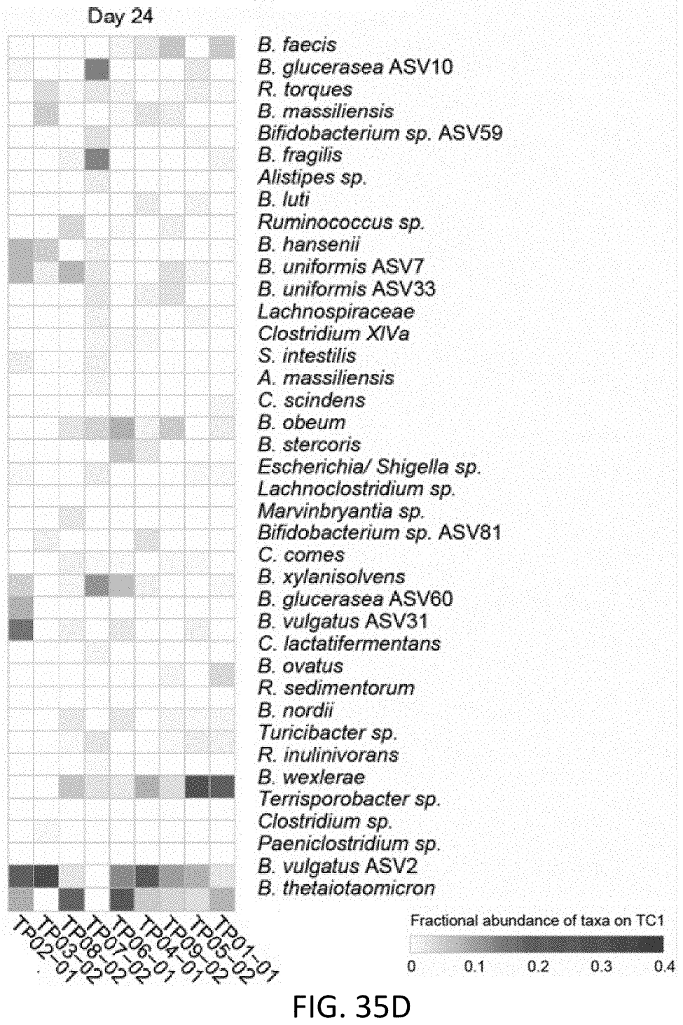

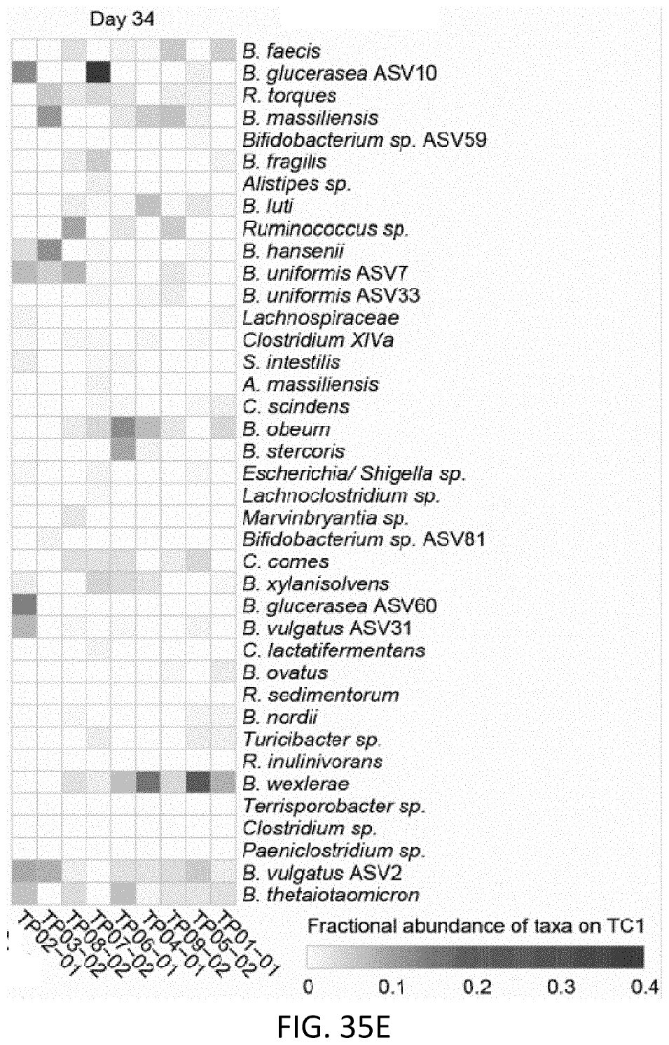

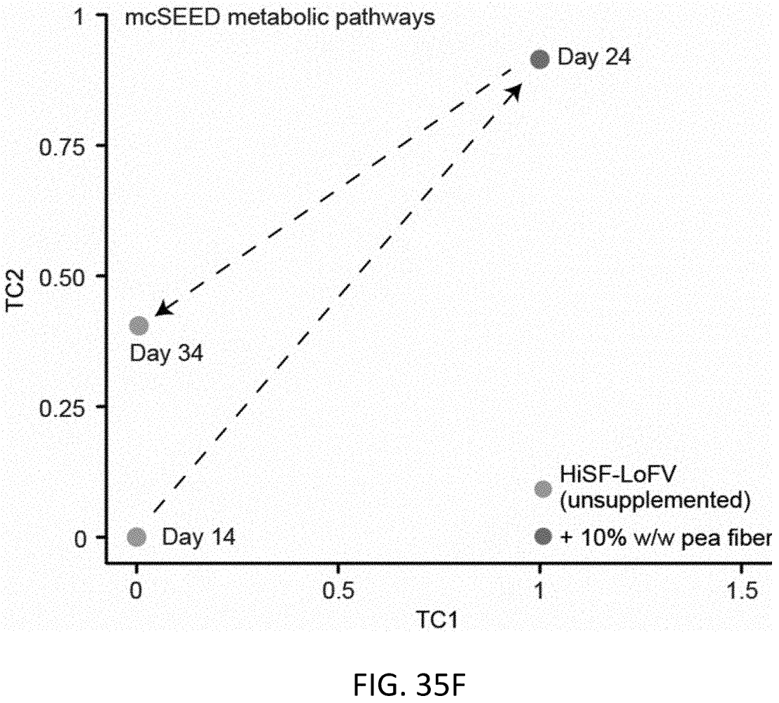

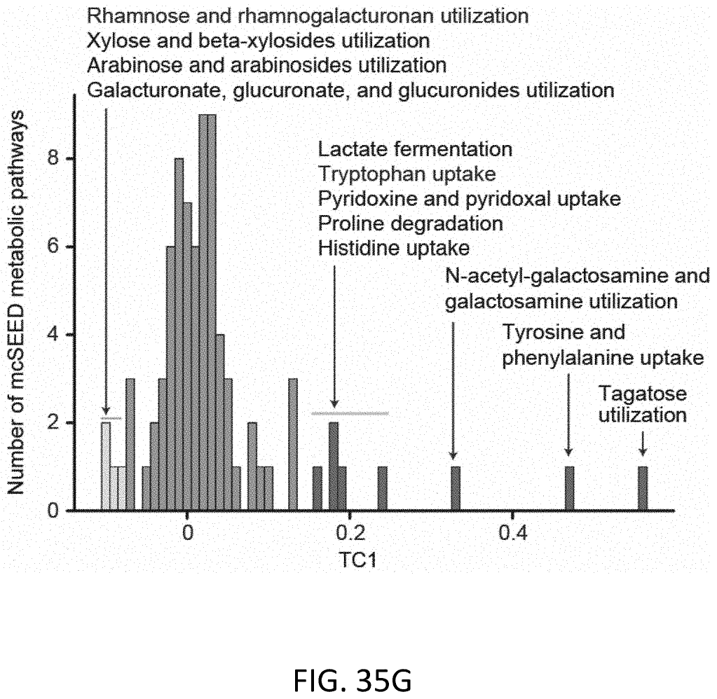

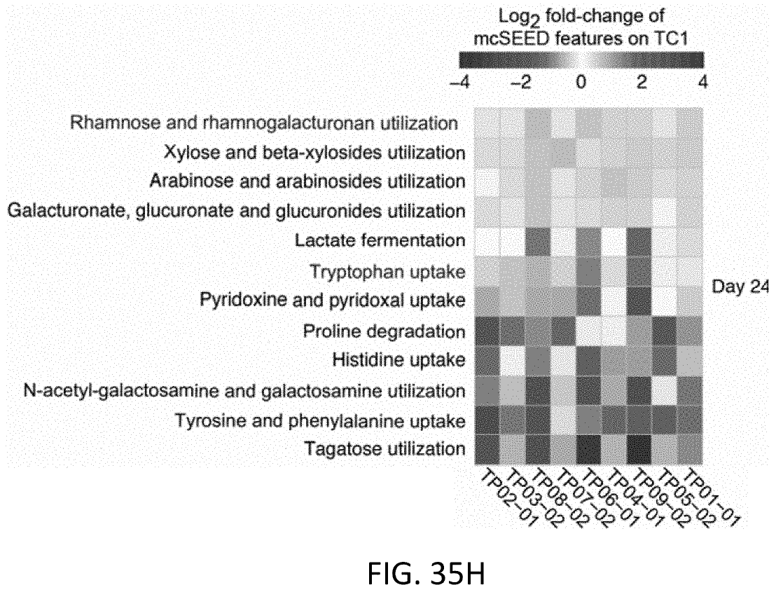

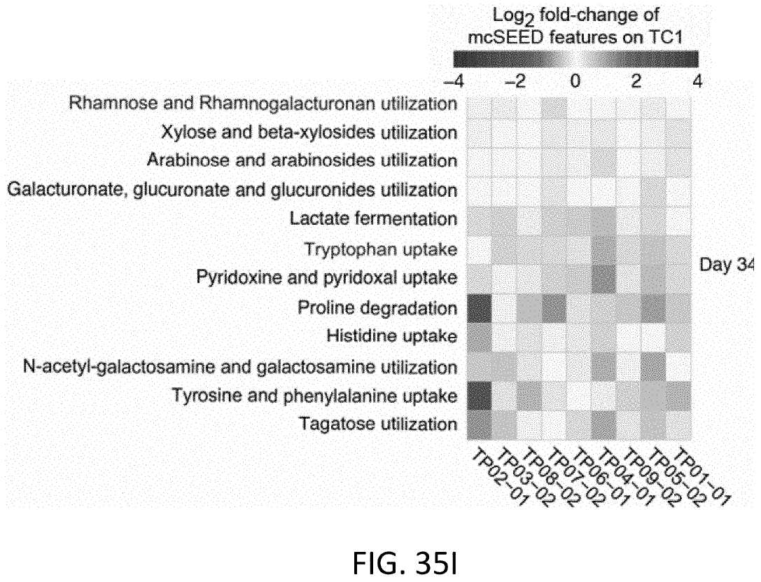

HO-SVD was used to evaluate the response to pea fiber by considering the initial three dietary phases (unsupplemented HiSF-LoFV on day 14, HiSF-LoFV plus pea fiber at day 24, and return to unsupplemented HiSF-LoFV on day 34) (FIG. 30C, FIG. 35. Fractional abundance values for ASVs were transformed by computing the log₂ fold-change from a reference time point (experimental day 14 when animals were consuming the unsupplemented HiSF-LoFV diet). HO-SVD was employed to identify a set of Tensor Components (TCs) that signified taxonomic variation due to pea fiber consumption. A ‘randomized tensor’ was generated by shuffling the rows (each mouse), columns (each ASV “taxon”), and z-axis (timepoints) of the tensor. The results disclosed that two tensor components encompassed non-random covariation between mice, taxa, and dietary condition. Each of the 57 mice included in the analysis, each of the 381 ASVs detected, and each of the three dietary phases (conditions) applied contribute towards, or ‘project on’, each TC. Plotting the ASV response to pea fiber revealed a pronounced effect on community structure (FIG. 35A). Ten days after withdrawal of pea fiber, the configuration of the microbiota had not fully returned to the pre-intervention state seen on day 14 (FIG. 35A). The projections of the three dietary phases on TC1 to TC2 illustrated that both tensors capture diet-dependent variation in the fractional abundance of bacterial taxa (FIG. 35A). FIG. 35B displays a histogram of taxonomic projections on TC1. The major drivers underlying this response were members of Bacteroides, including Bacteroides thetaiotaomicron and Bacteroides vulgatus (FIG. 35B). The heatmap in FIG. 35C-E shows the increases in their abundance upon exposure to pea fiber, with the extent of change varying between mice gavaged with the different human donor microbiota.

A comparable HO-SVD-based study of genes encoding CAZymes disclosed pronounced configurational changes in transplanted donor microbiomes after pea fiber supplementation (FIG. 30C), including increases in the representation of genes encoding arabinosidases belonging to glycoside hydrolase family 43 (GH43_2, GH43_9, GH43_17, GH43_18 and GH43_19 subfamilies contain alpha-L-arabinofuranosidases) (FIG. 30D,E). This finding suggested that arabinan is a predominant polysaccharide in pea fiber utilized by several human gut Bacteroides species (15). In addition, pea fiber-associated increases in the representation of genes encoding galactosidases (GH43_3, GH43_8, GH43_31 families contain beta-D-galactofuranosidases) and beta-1,4-glucanases belonging to GH family 5 (GH5_1, GH5_4, GH5_5, and GH5_38) were identified (FIG. 30E). HO-SVD also disclosed alterations in the abundances of mcSEED pathways for utilization of monosaccharides that are prominently represented in pea fiber (arabinose, xylose, rhamnose, and galacturonic acid; see FIG. 35F-I). Notably, the degree of interpersonal variation in the response to pea fiber when defined at the level of the microbiome (CAZymes and mcSEED metabolic pathways) was less than that defined at the level of the microbiota (ASVs) (compare FIG. 30E with FIG. 35C-E,H, I).

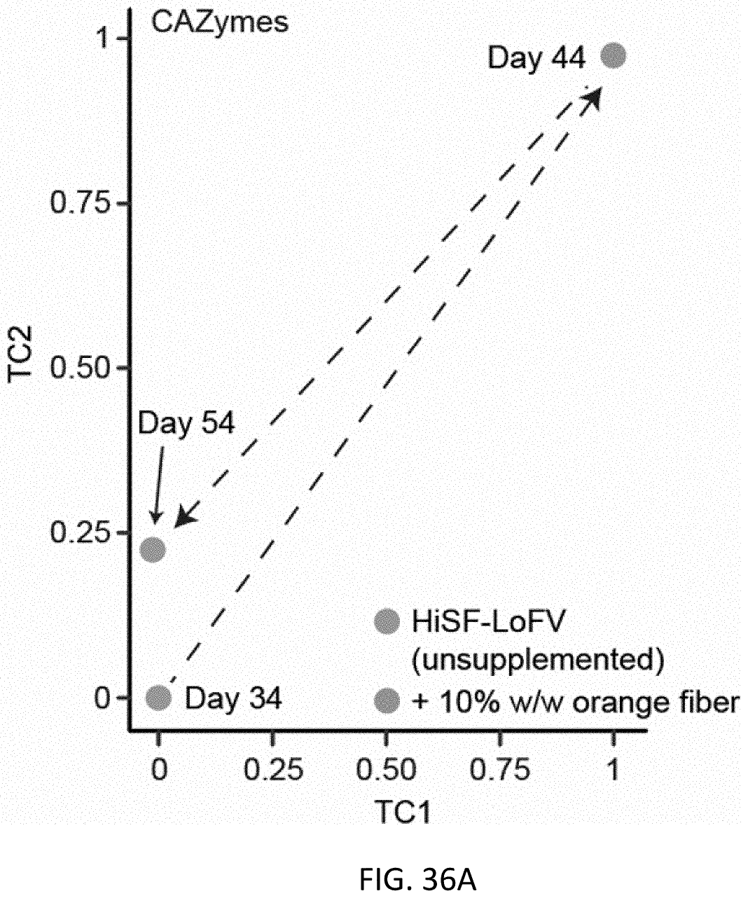

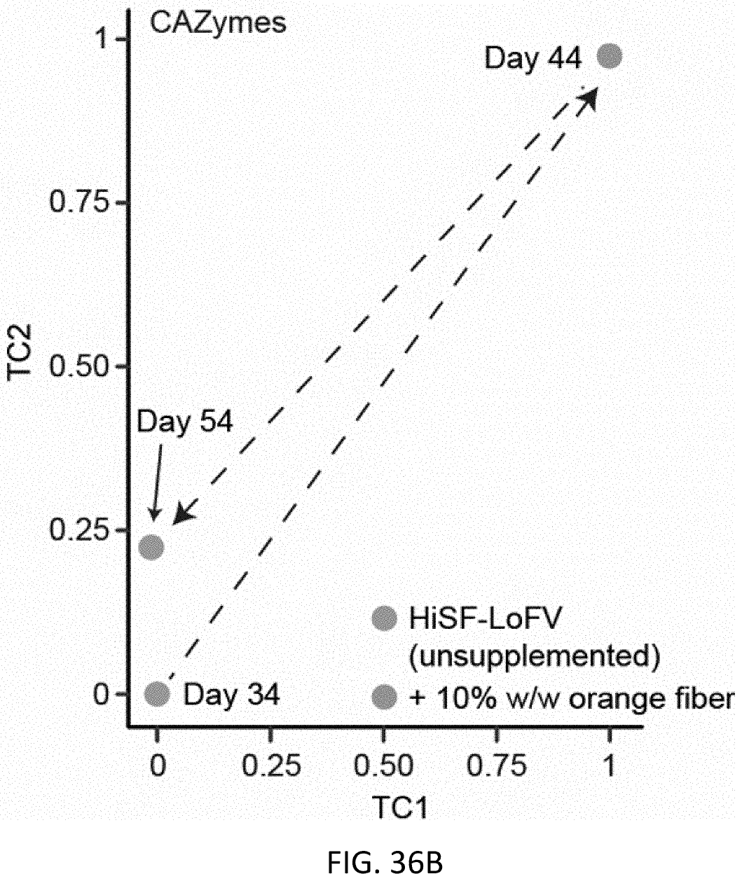

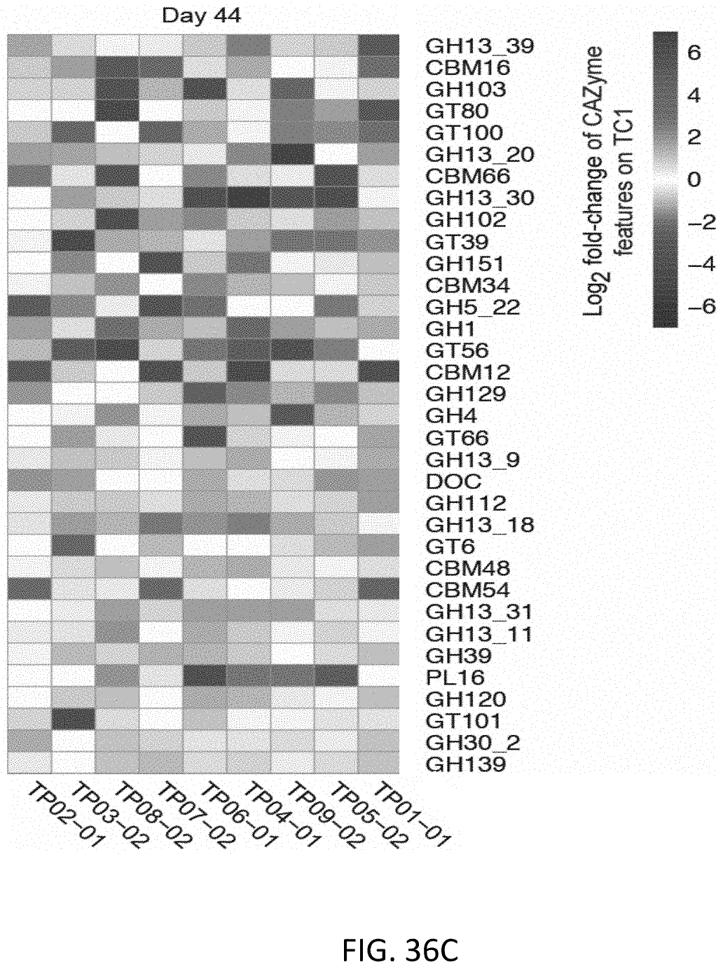

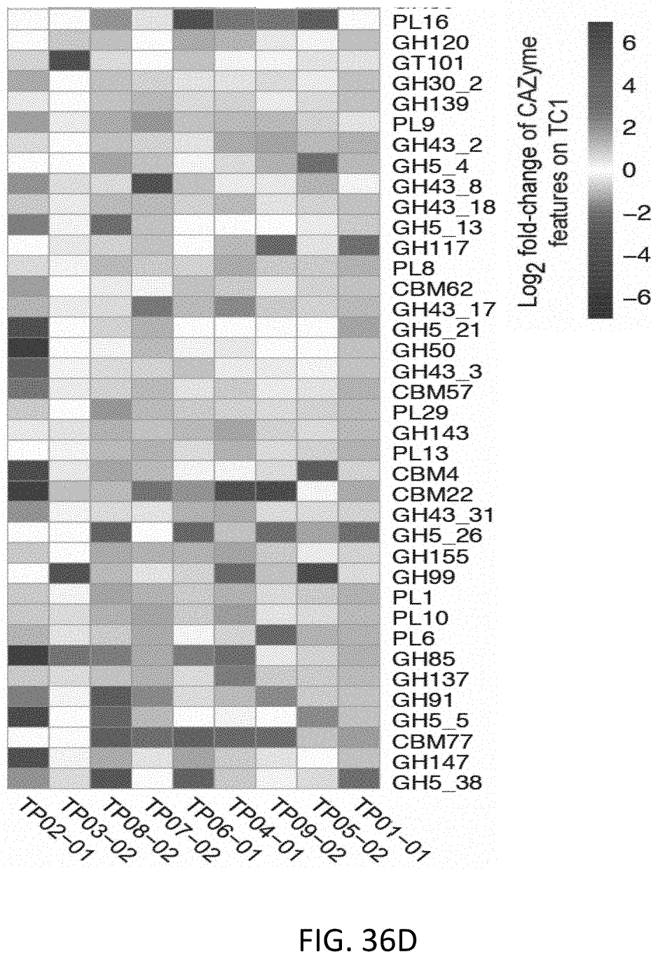

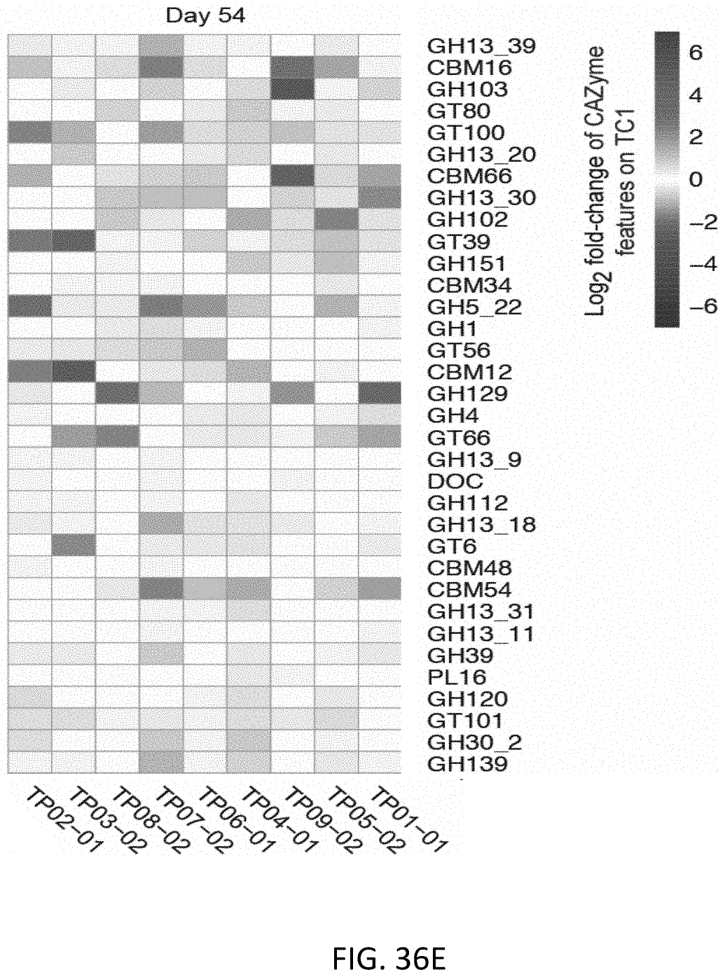

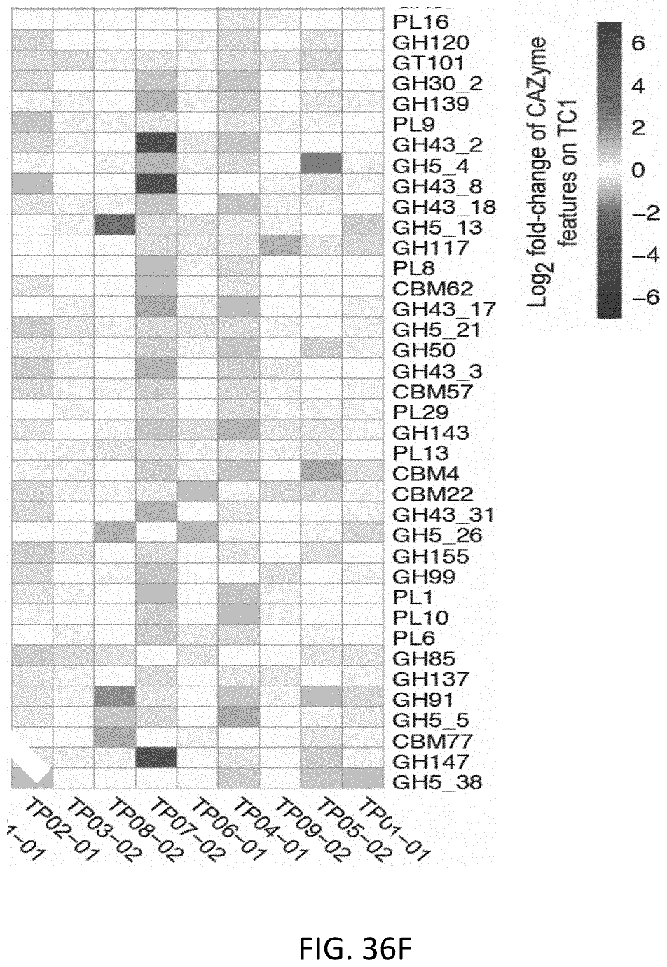

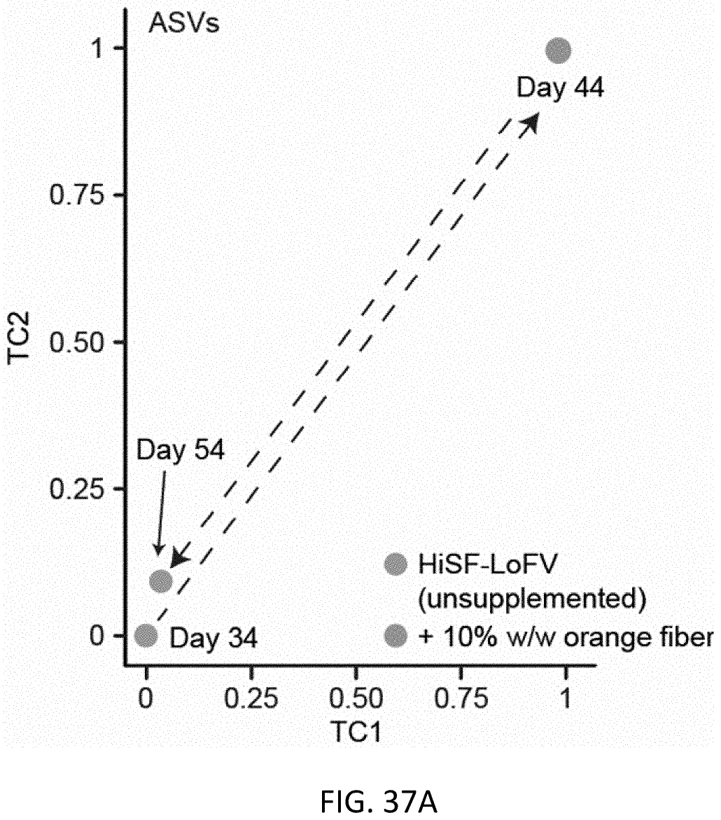

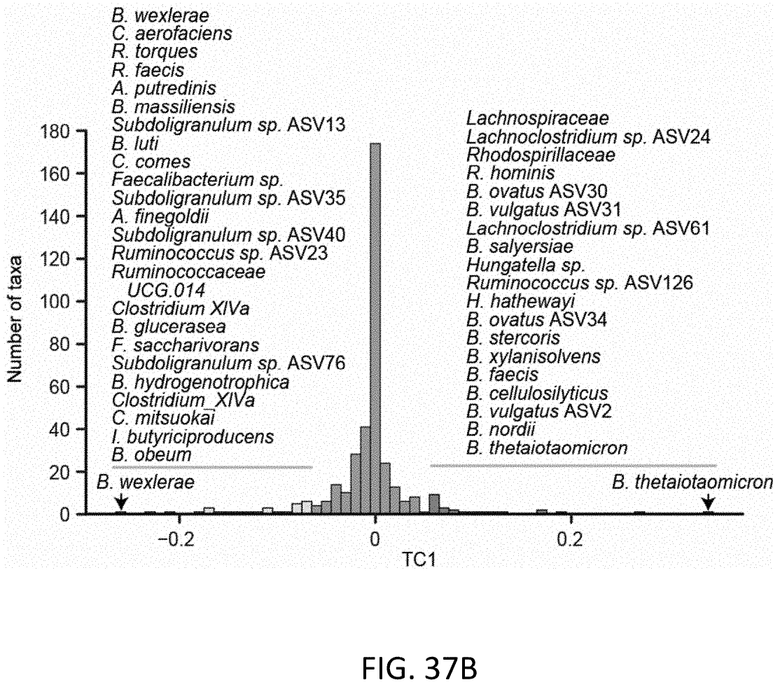

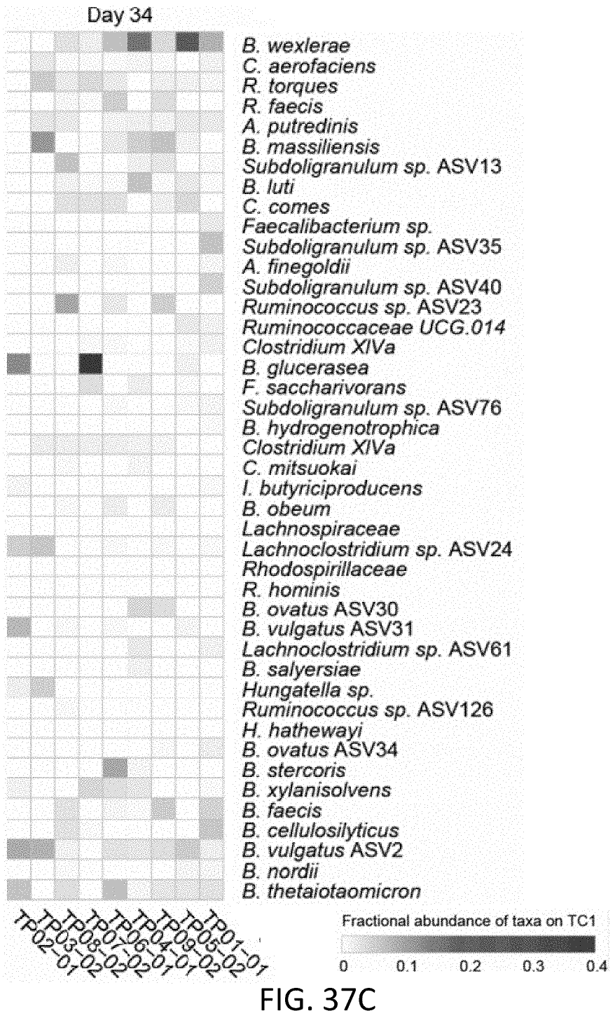

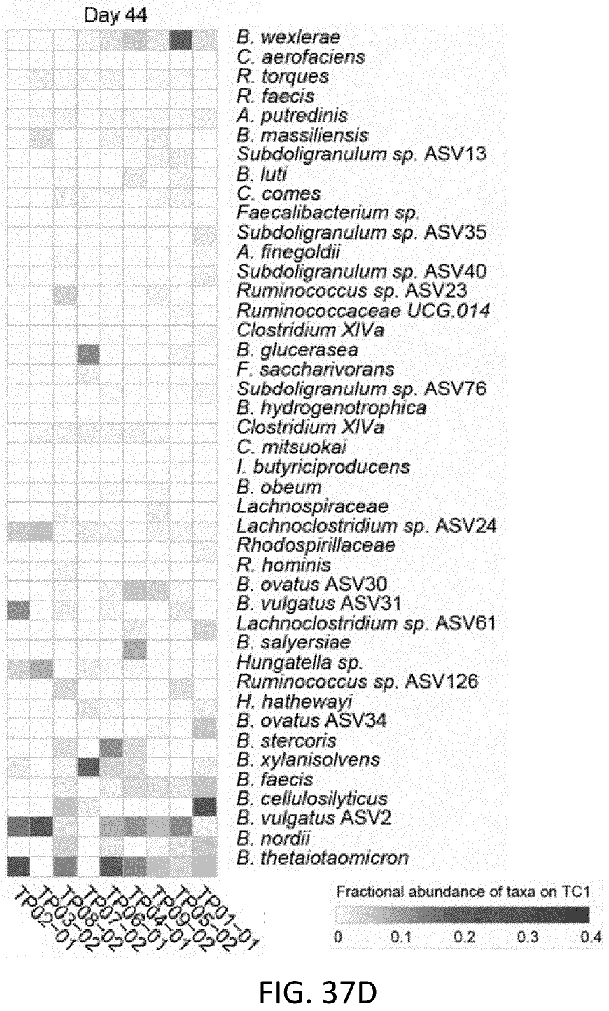

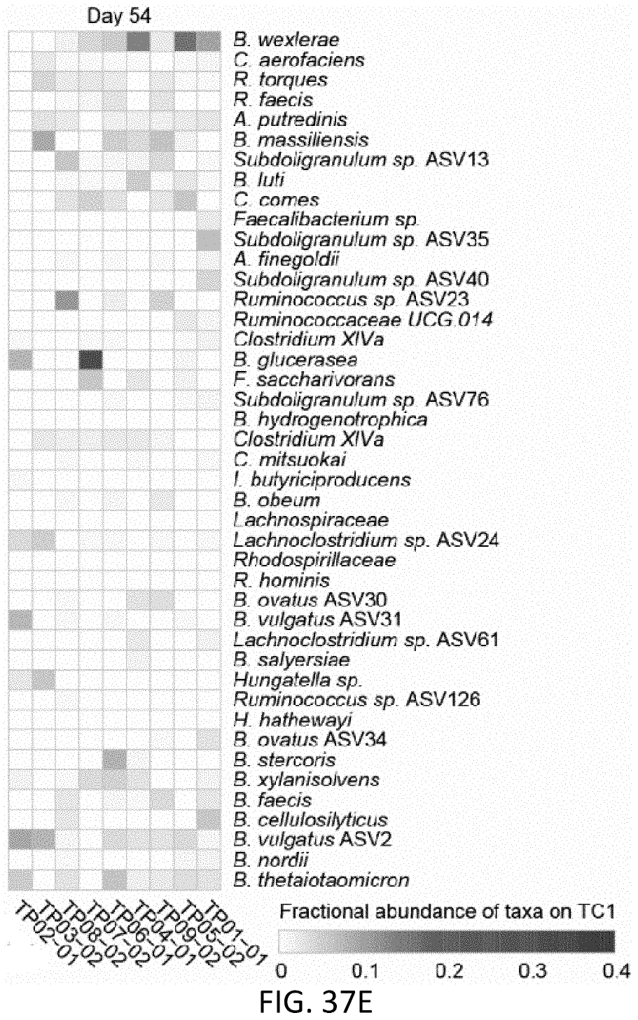

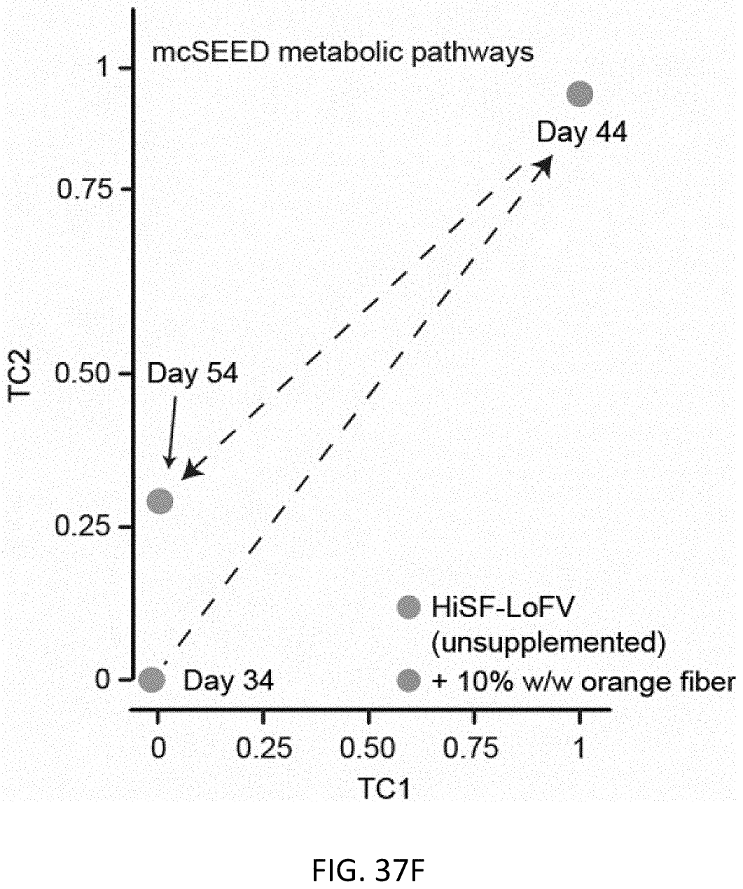

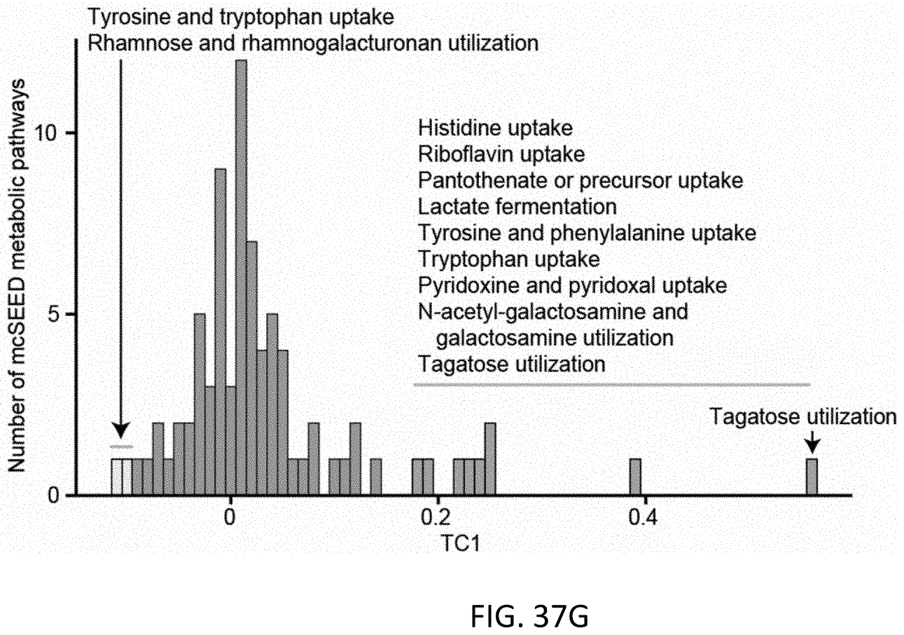

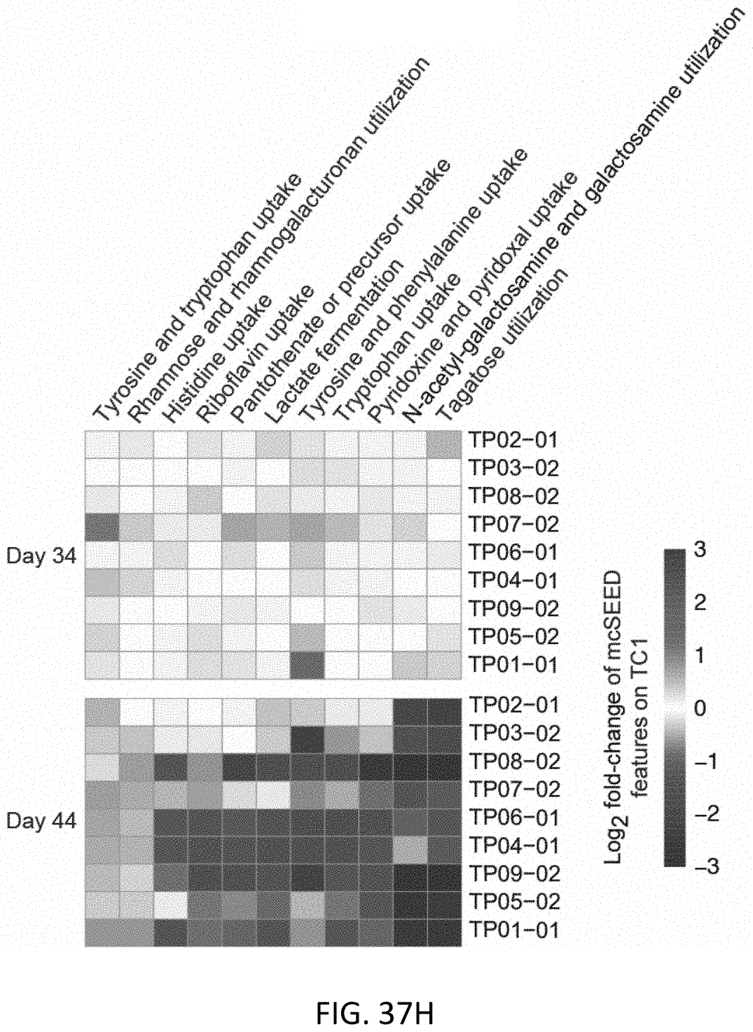

FIG. 36-39 present the results of a comparable HO-SVD study of the effects of orange fiber and barley bran. Consistent with the similarities in polysaccharide composition between pea fiber and orange fiber, fecal microbiomes sampled during orange fiber administration revealed increased representation of genes involved in the processing of arabinan (GH43_2, GH43_17, GH43_18), galactan (GH43_3, GH43_8, GH43_31), and galacturonans (PL1, PL9, PL10) (FIG. 36B,C), as well as genes involved in the mcSEED pathway for rhamnose utilization (FIG. 37G,H). As was the case with pea fiber, B. thetaiotaomicron and B. vulgatus were the major drivers underlying the orange fiber response, in addition to Bacteroides nordii (FIG. 37B,C,D,E). Similar to pea fiber, the degree of interpersonal variation in community response to orange fiber was less in CAZyme, and mcSEED metabolic pathway feature space than ASV-feature space (compare FIG. 36C,D,E,F and FIG. 37CD, E, H).

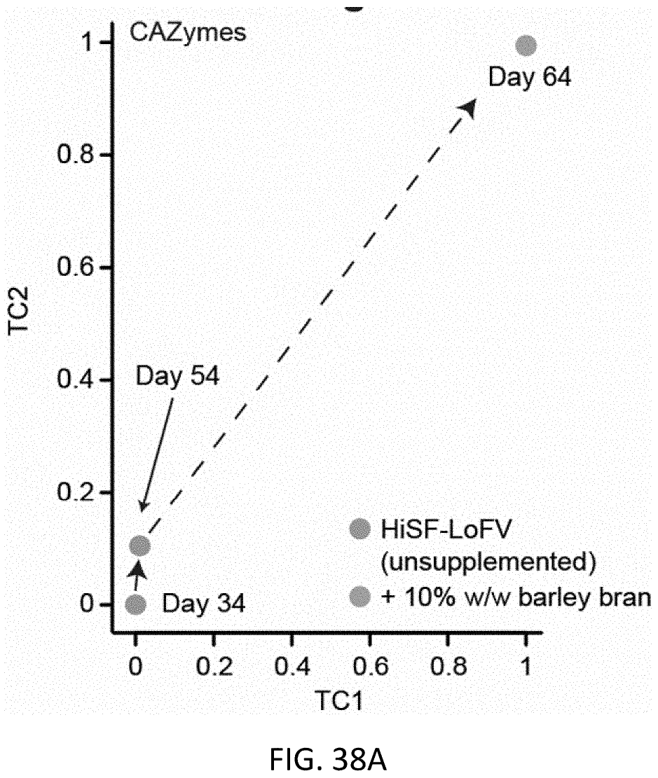

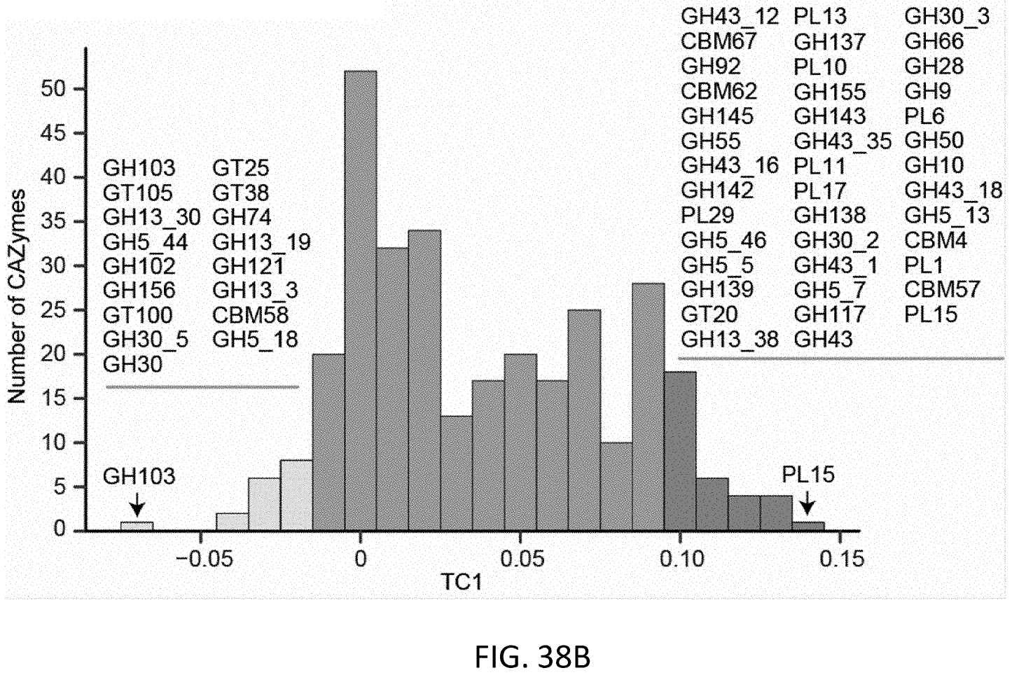

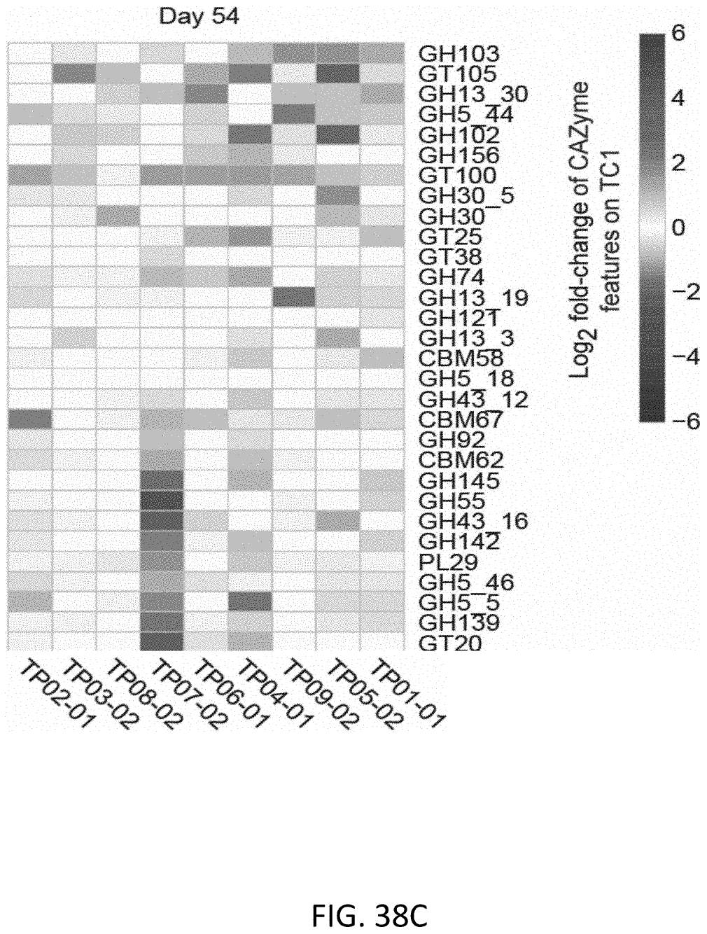

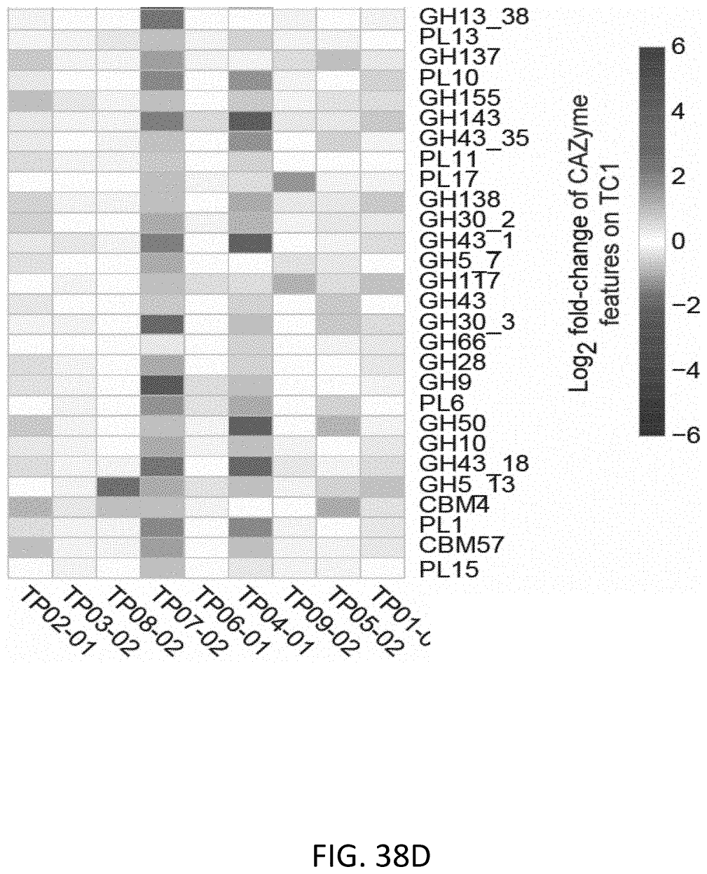

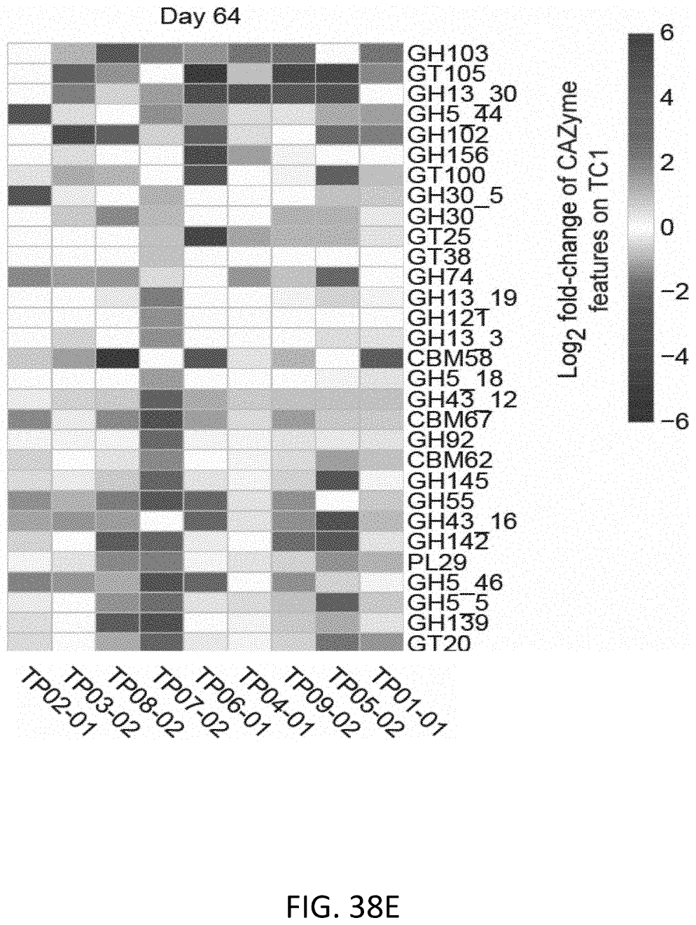

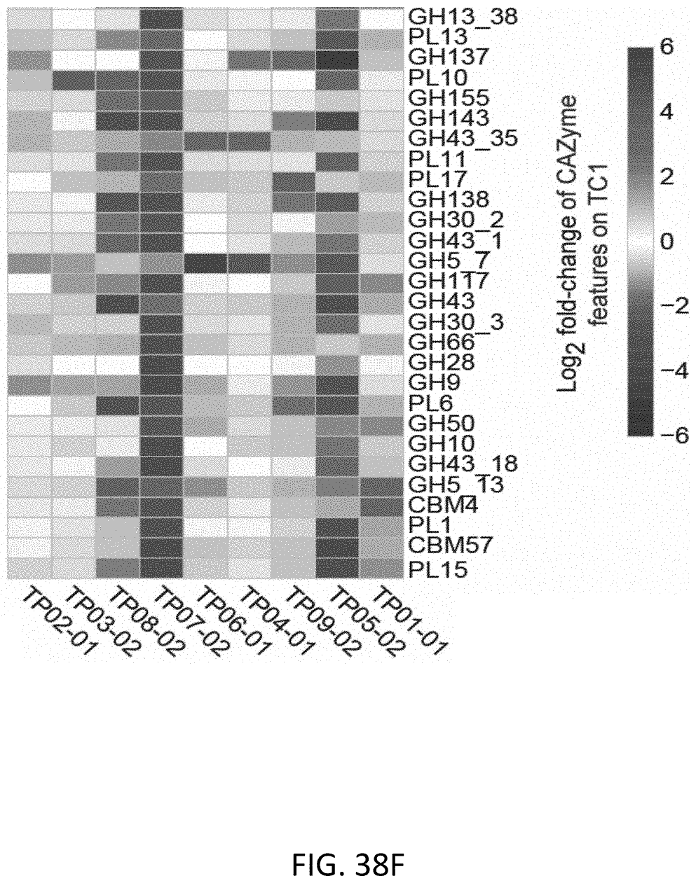

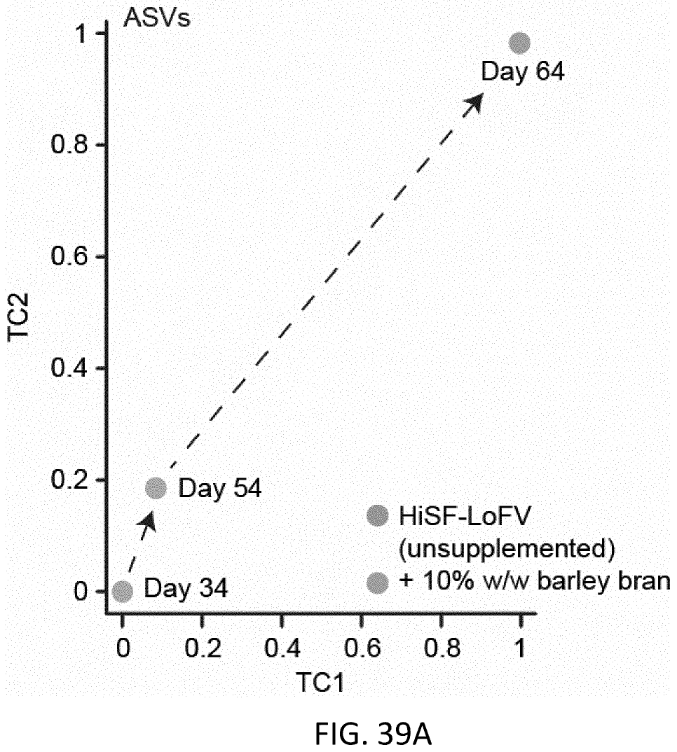

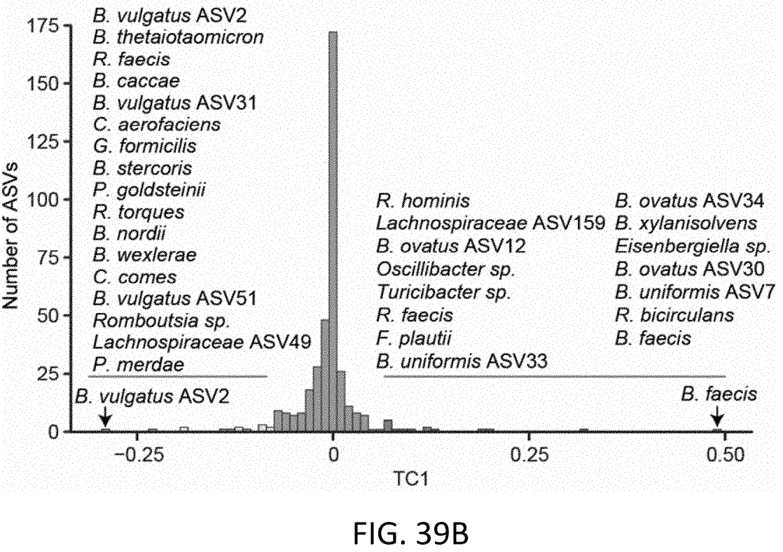

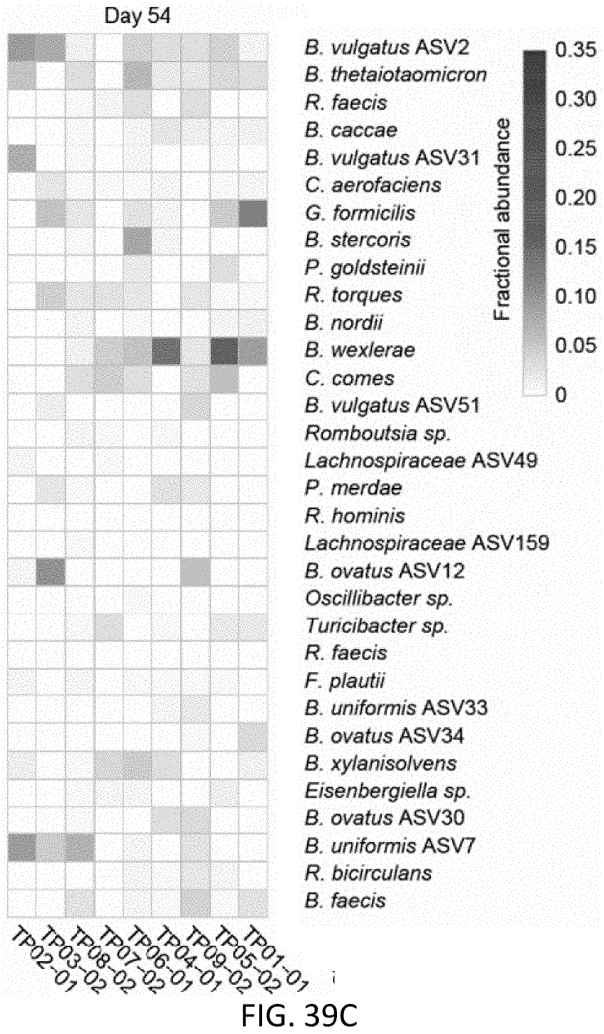

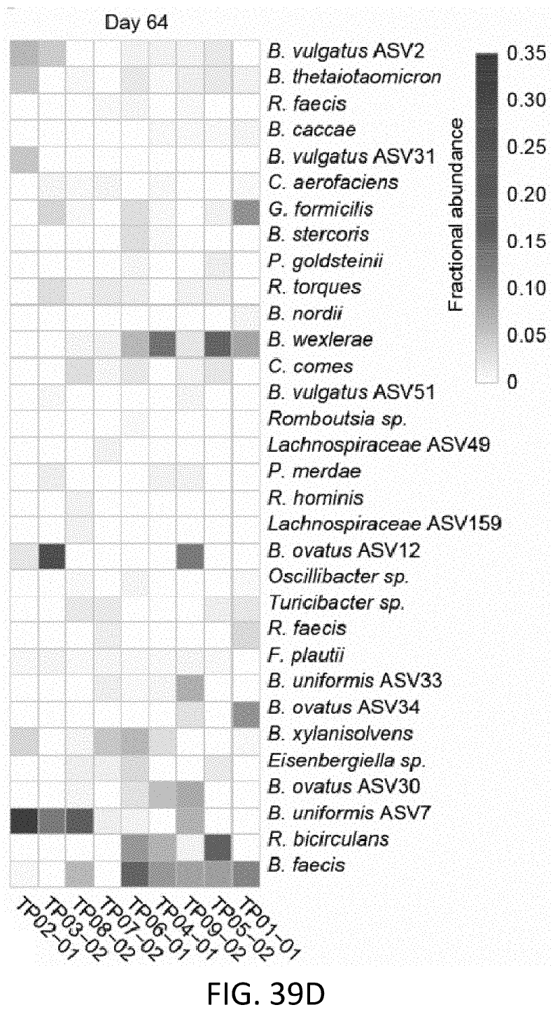

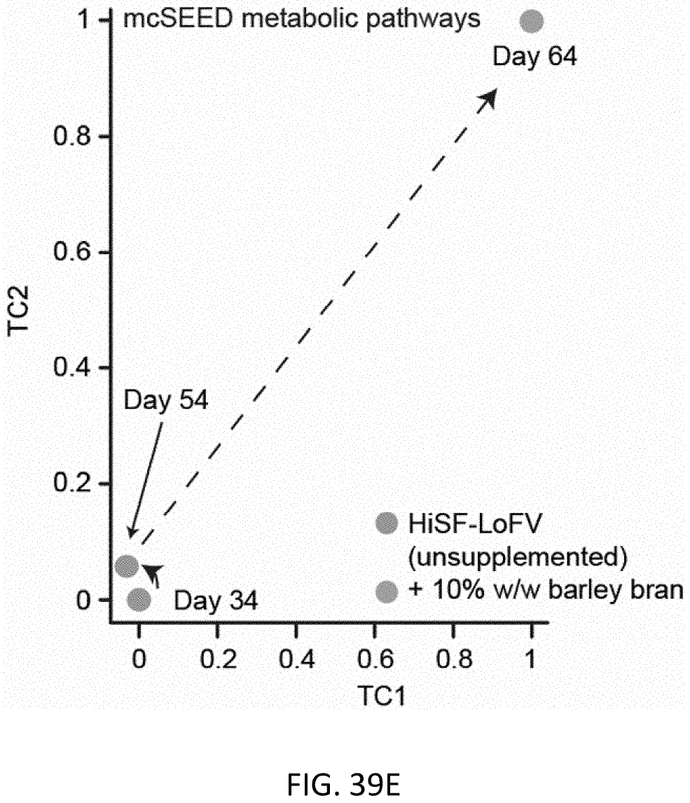

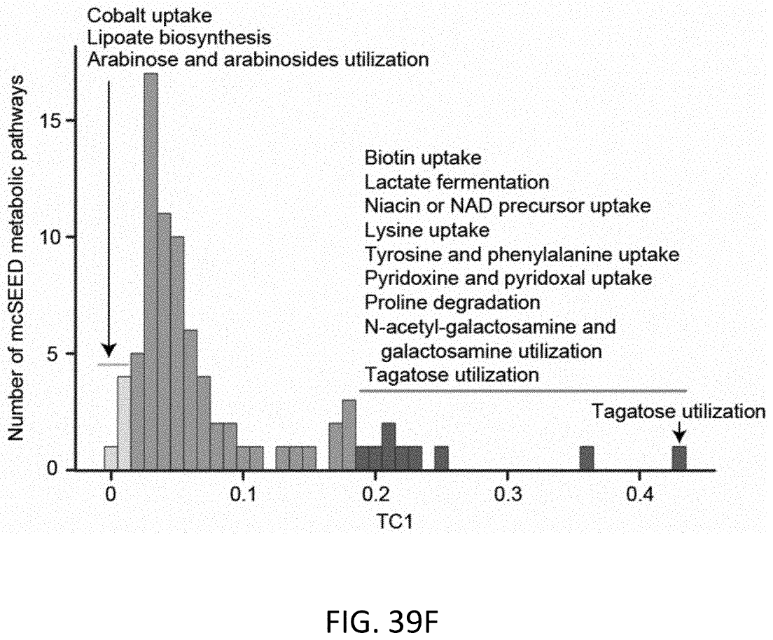

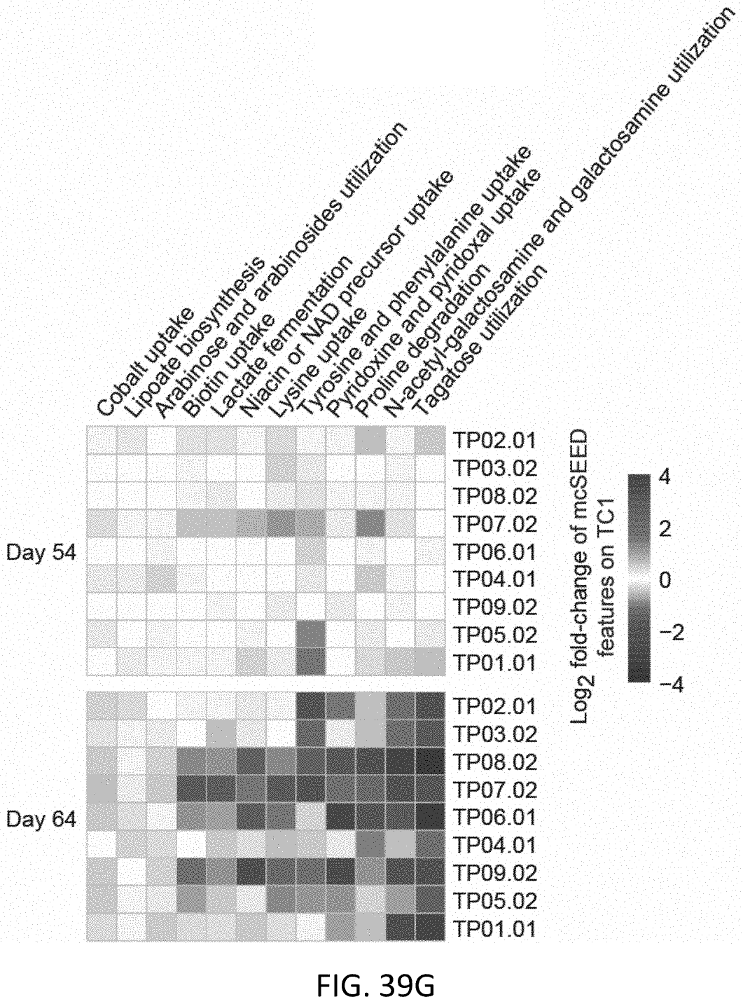

Barley bran produced increases in the representation of genes involved in the processing of beta-glucans (GH5_5, GH5_46), arabinoxylans (GH43_1, GH43_12, GH43_16, GH43_35), and galacturonans (PL1, PL10, PL11) (FIG. 38B,C,D,E,F), as well as genes involved mcSEED pathways for arabinose and arabinosides utilization (FIG. 39E-G). In contrast to pea and orange fibers, Blautia faecis, Ruminococcus bicirculans, Bacteroides uniformis, and Bacteroides ovatus, were the major drivers of the response to barley bran consumption (FIG. 39B,C,D).

### Example 14. Testing Fiber-Containing Snack Food in Overweight or Obese Adults

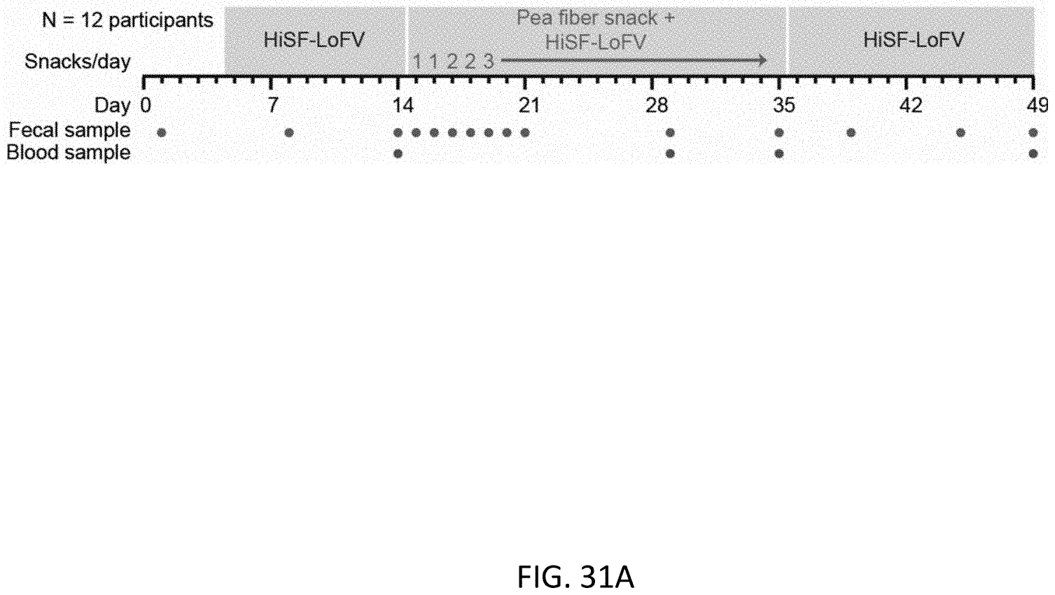

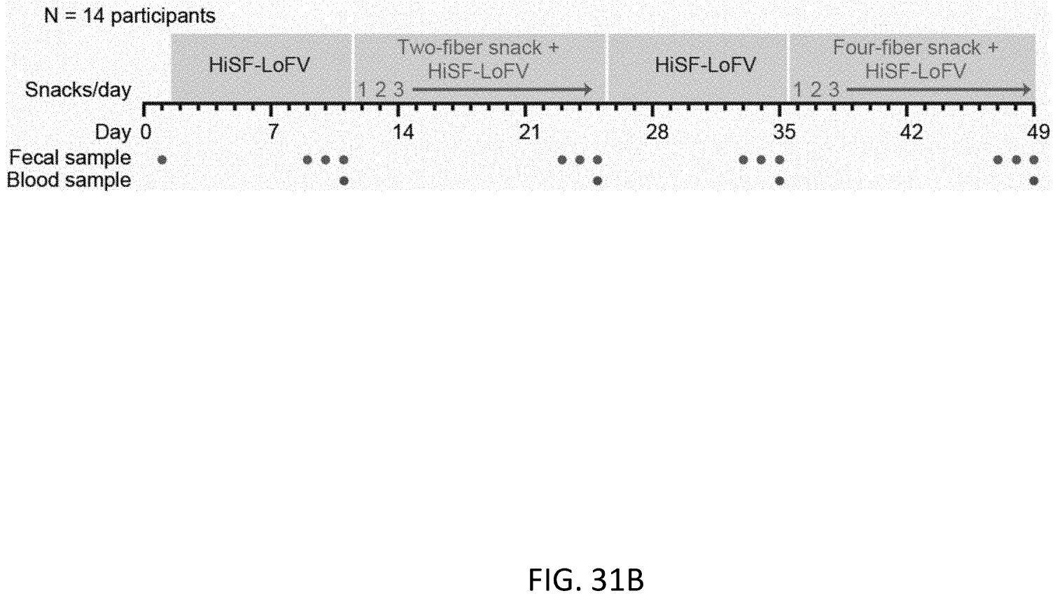

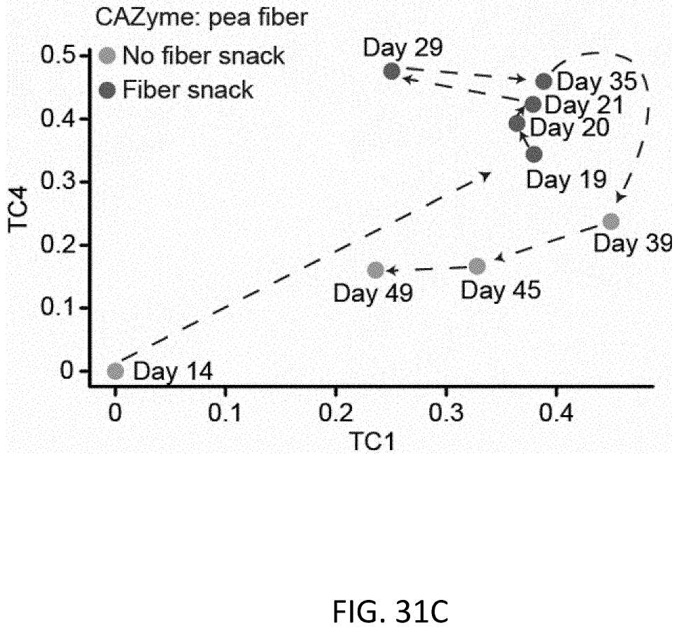

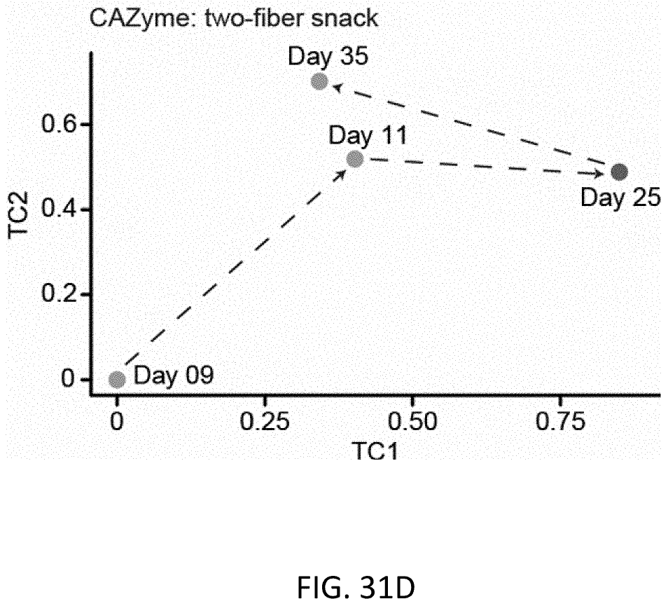

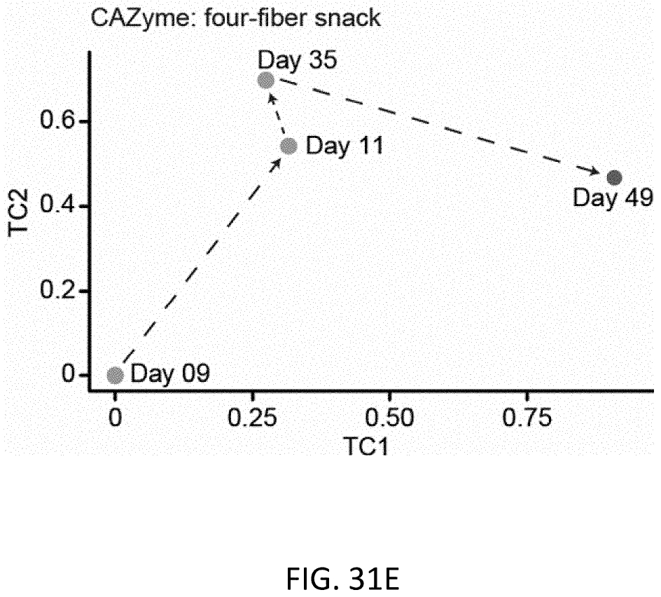

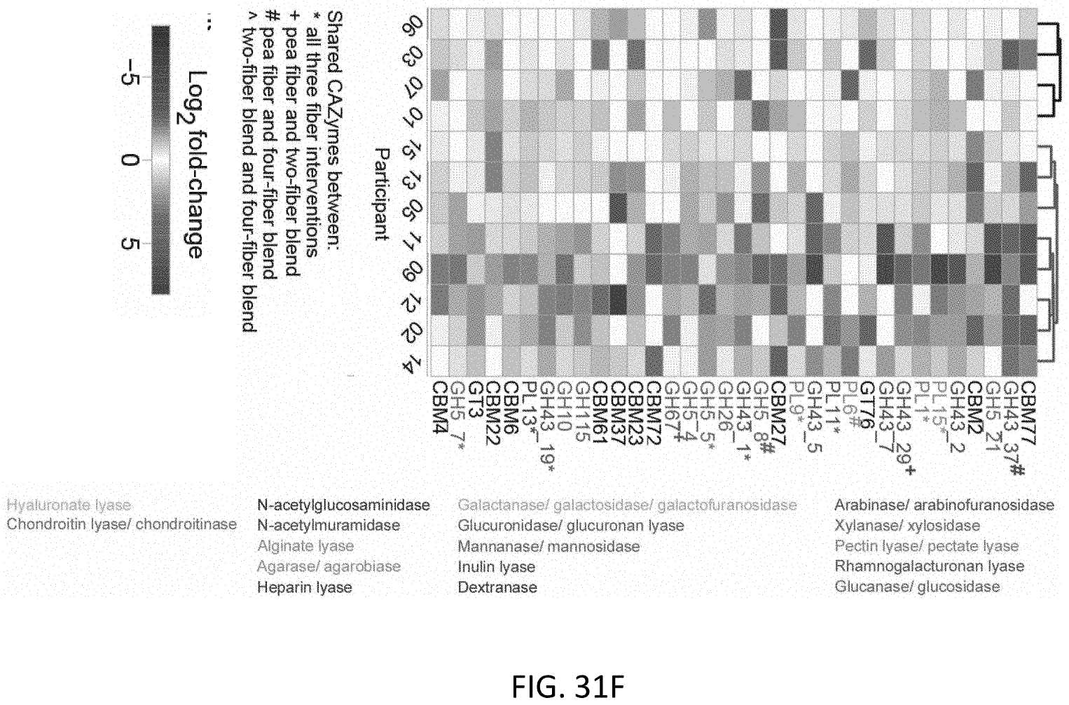

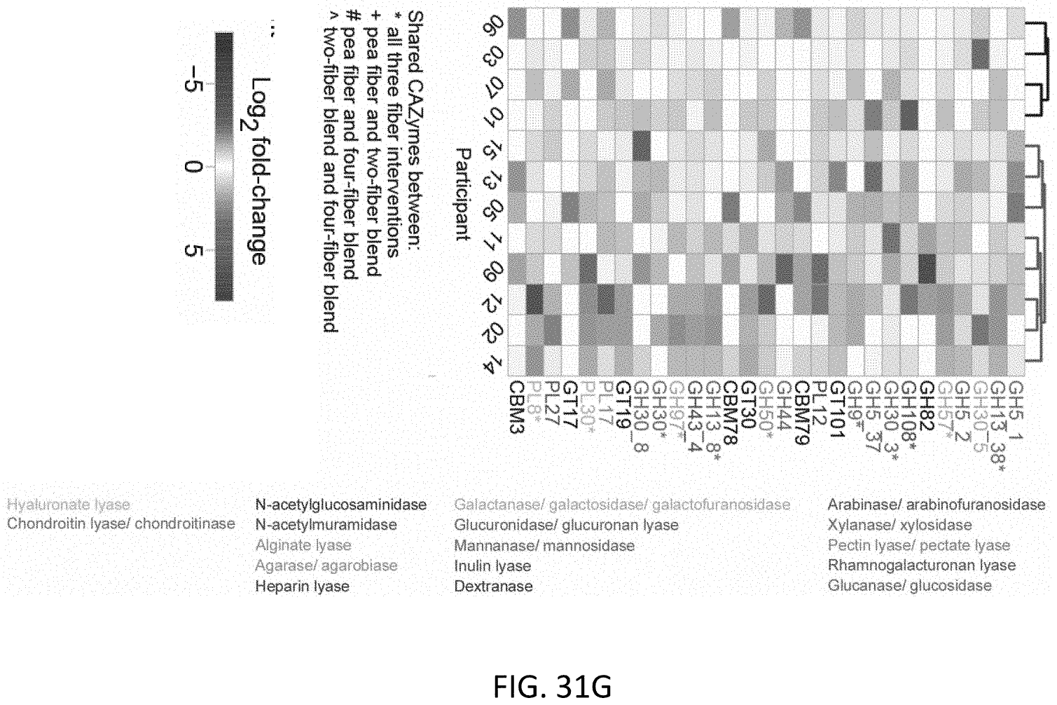

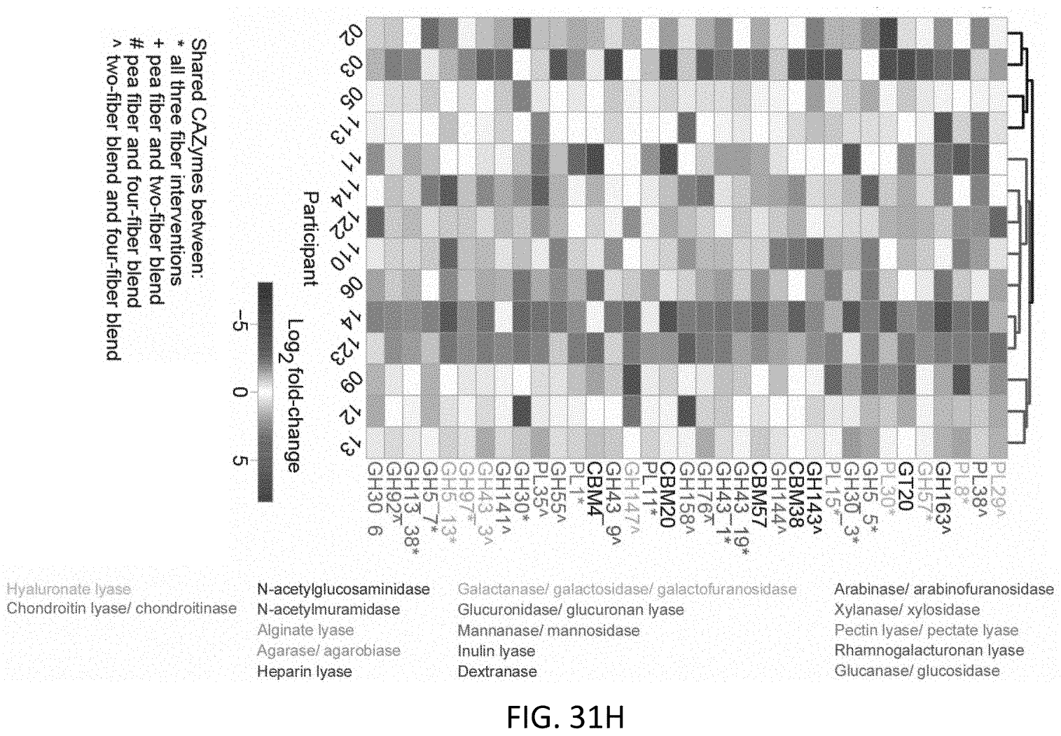

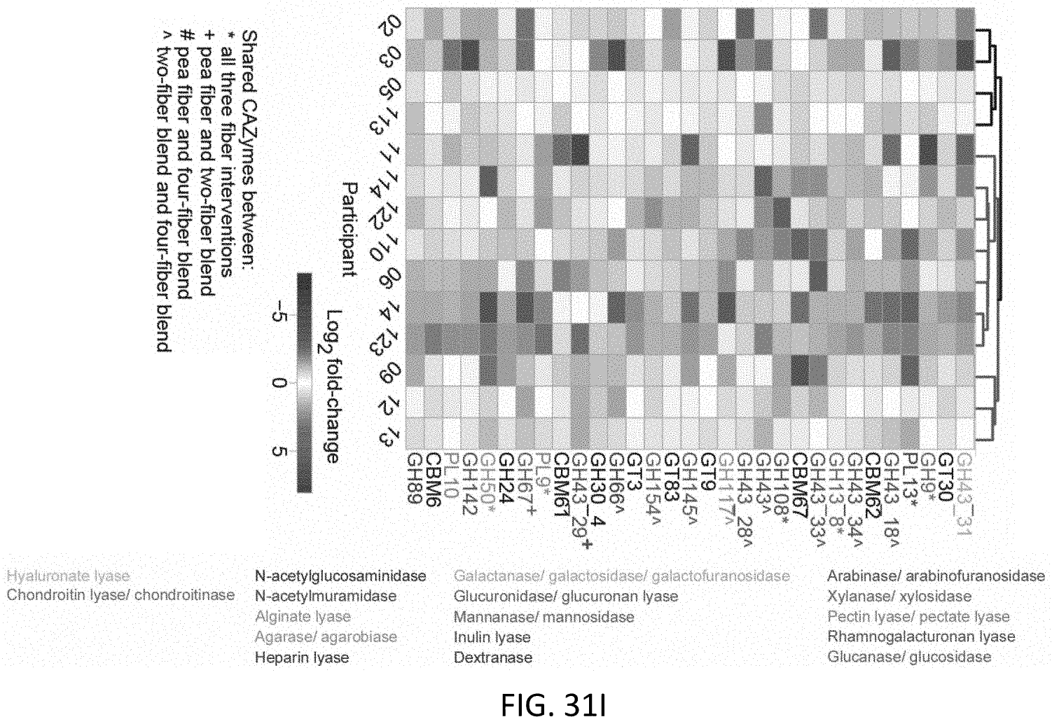

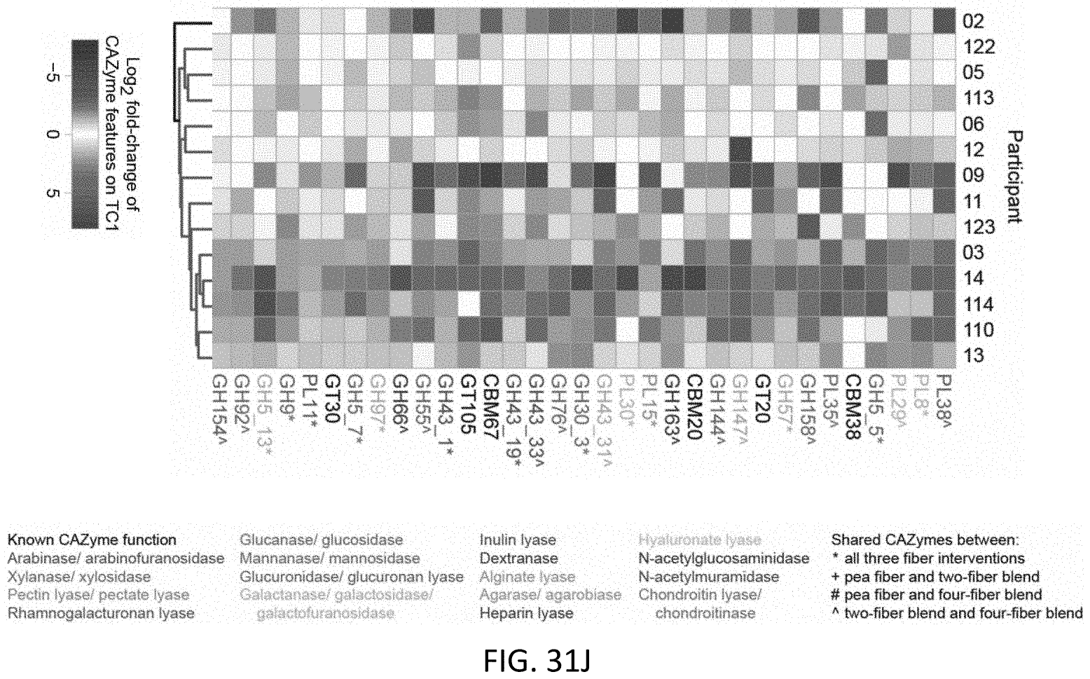

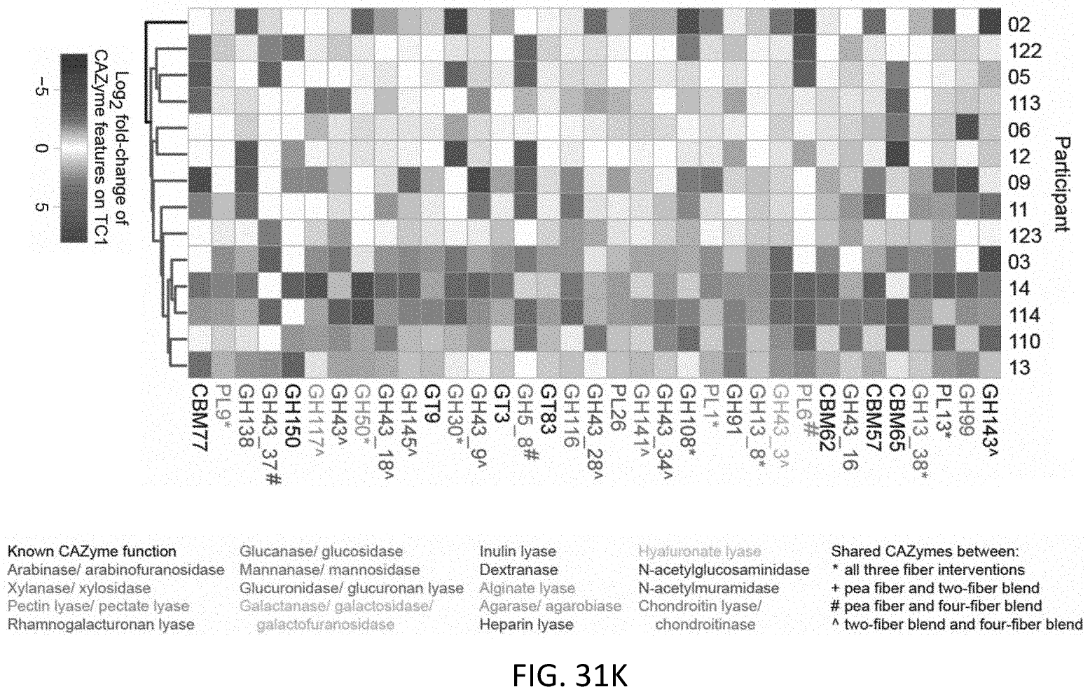

To assess the degree to which results obtained from gnotobiotic mice were translatable to humans, we performed a controlled diet study involving 12 participants who were overweight or obese and a food prototype containing pea fiber (see Table 21A for the snack formulation and Table 28 for a description of the subjects). Each participant provided a fecal sample while on their normal diet during the first four days of the study. Participants then followed a 45-day regimen where their normal diet was replaced with the equivalent of the HiSF-LoFV diet (Table 21B). Each 35 gram snack (Table 21A) contained 8.1 grams of extruded pea fiber (see Table 26 or the monosaccharide and glycosidic linkage composition of the extruded fiber preparation). Energy intake from the HiSF-LoFV diet was reduced to account for the energy provided by the snacks, so that overall energy intake was constant. Participants were followed for 14 days after stopping snack consumption, while still continuing the HiSF-LoFV diet (post-intervention ‘washout phase’). Daily body weight was monitored using “smart scales” which used cellular networks to send the data to the research team. No additional adjustments in the amount of the HiSF-LoFV diet consumed were needed to maintain a constant body weight during or after the period of treatment with the fiber snack. At various time points during the study, blood samples were obtained for clinical chemistry and plasma proteomic analyses, while fecal samples (n=202) were collected for V4-16S rDNA amplicon and whole community shotgun sequencing (FIG. 31A).

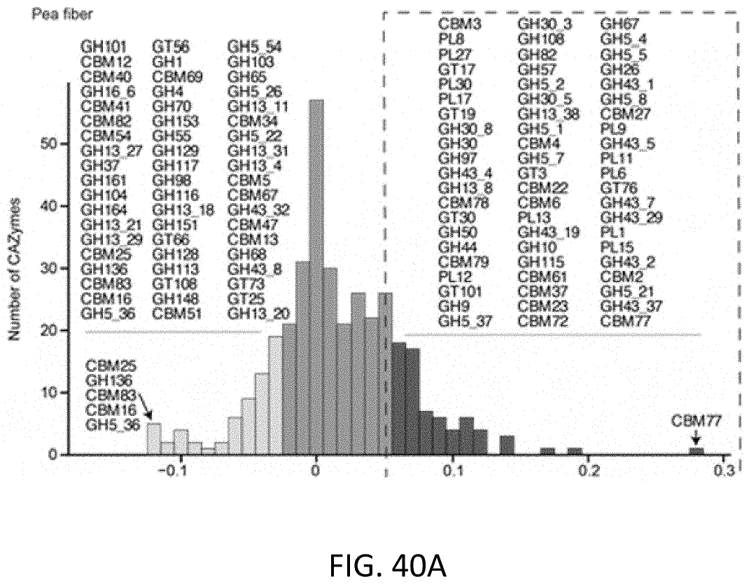

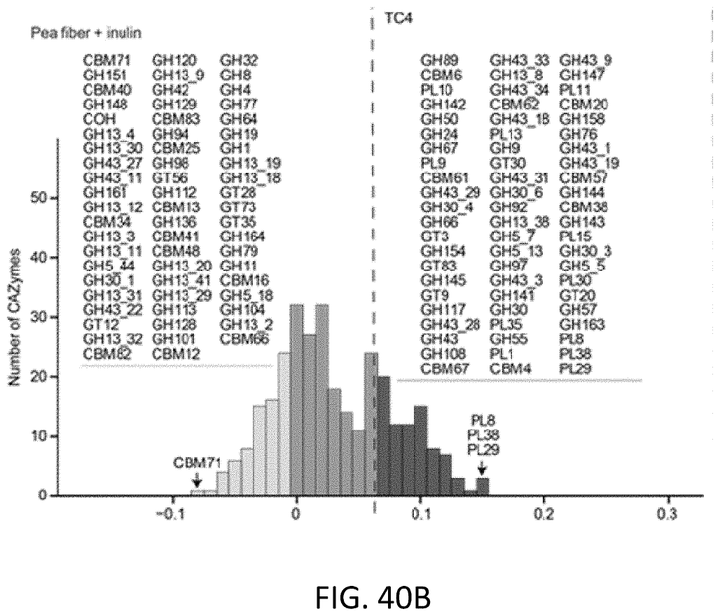

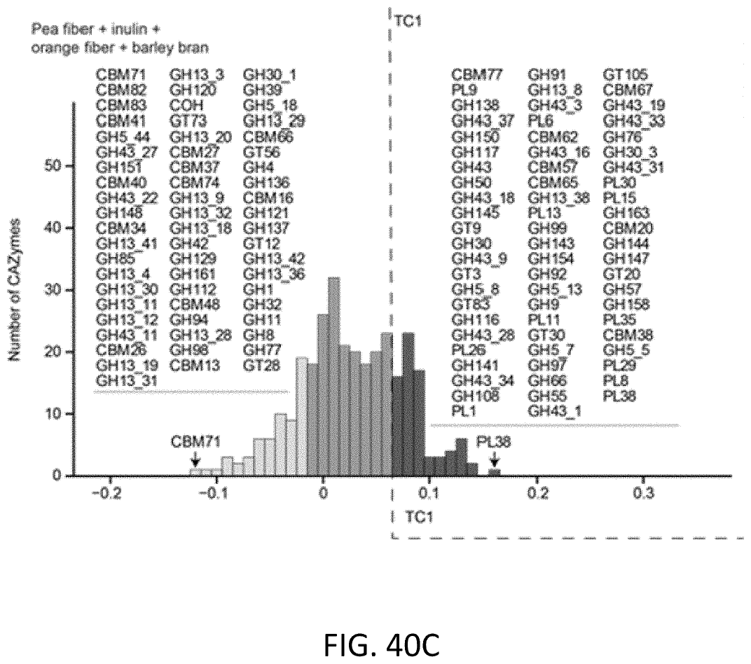

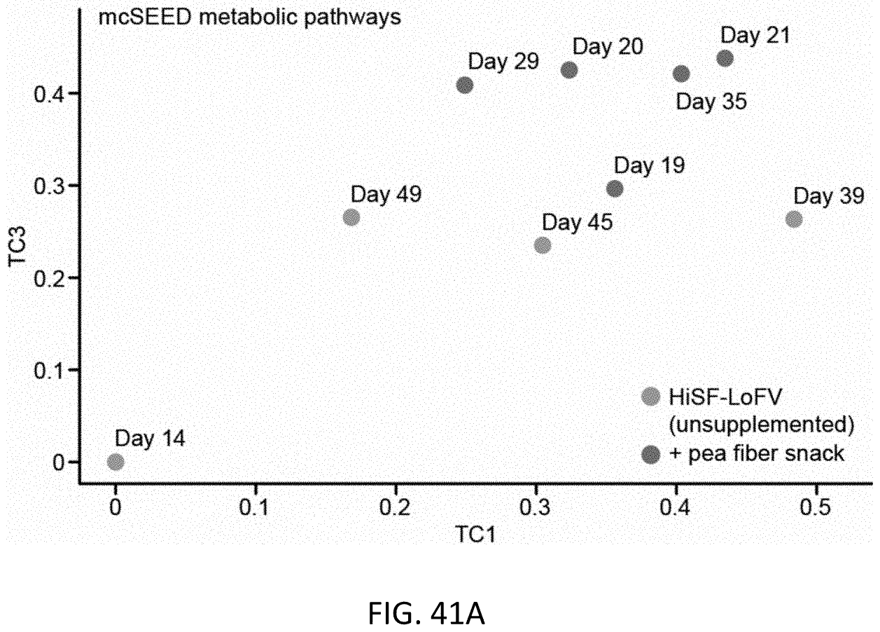

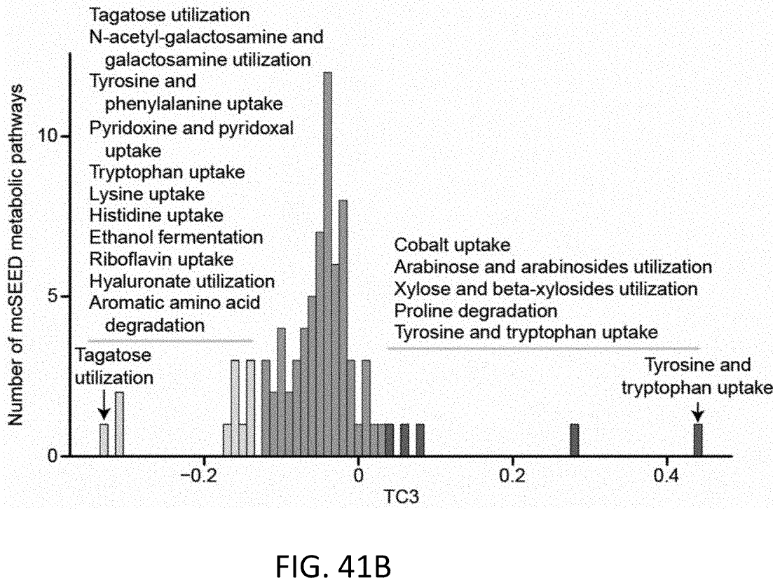

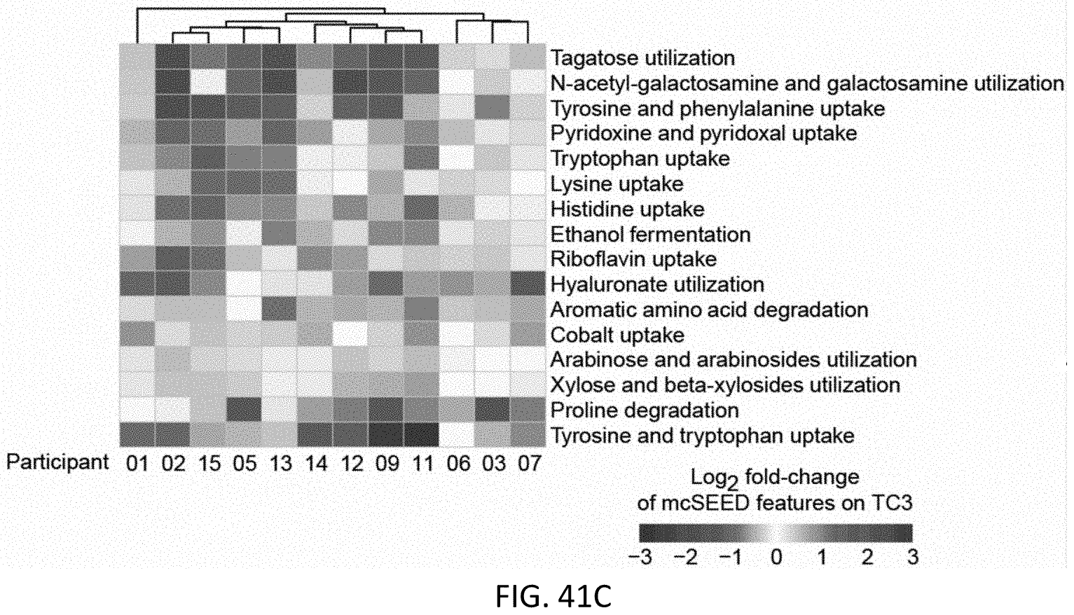

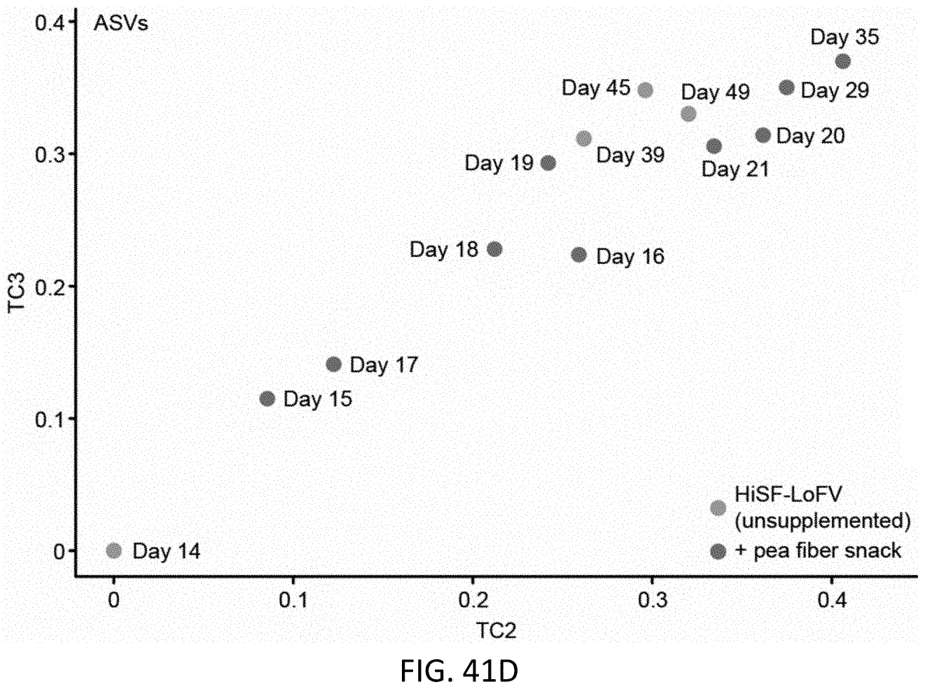

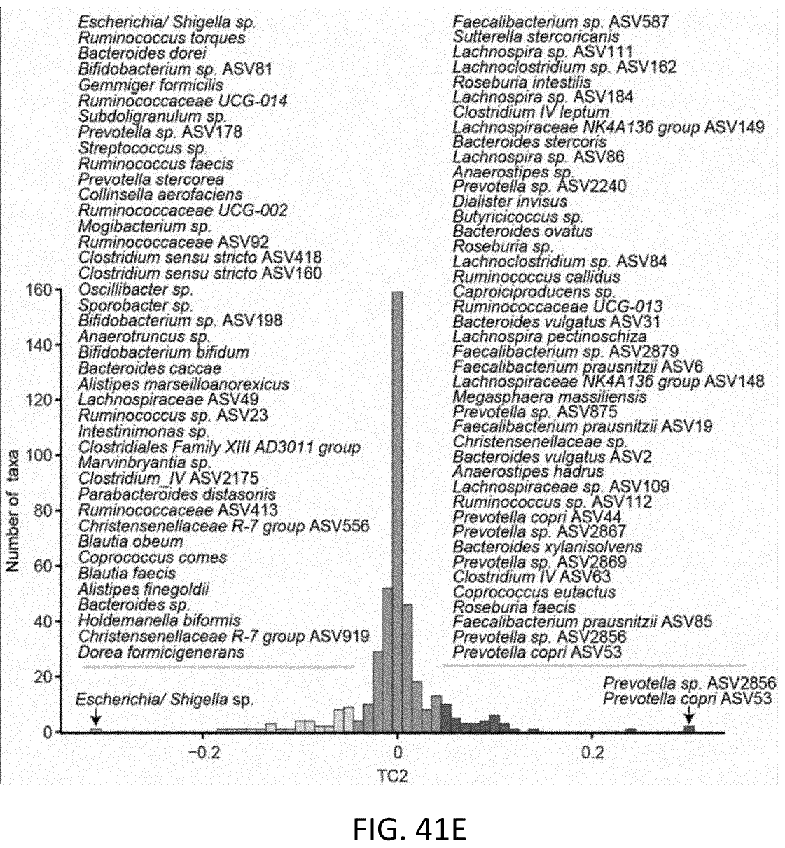

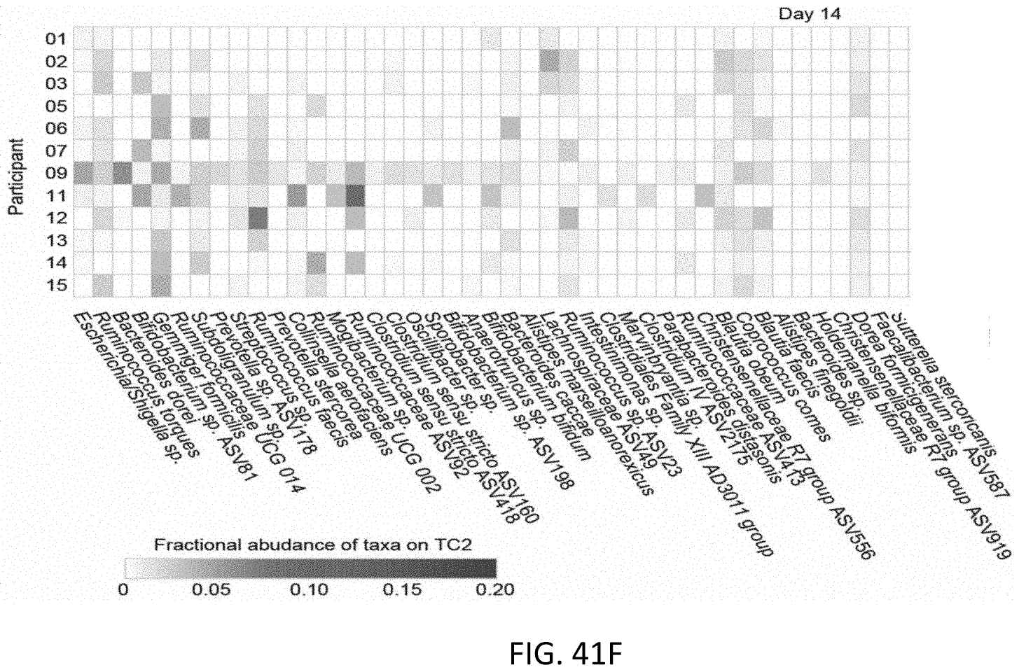

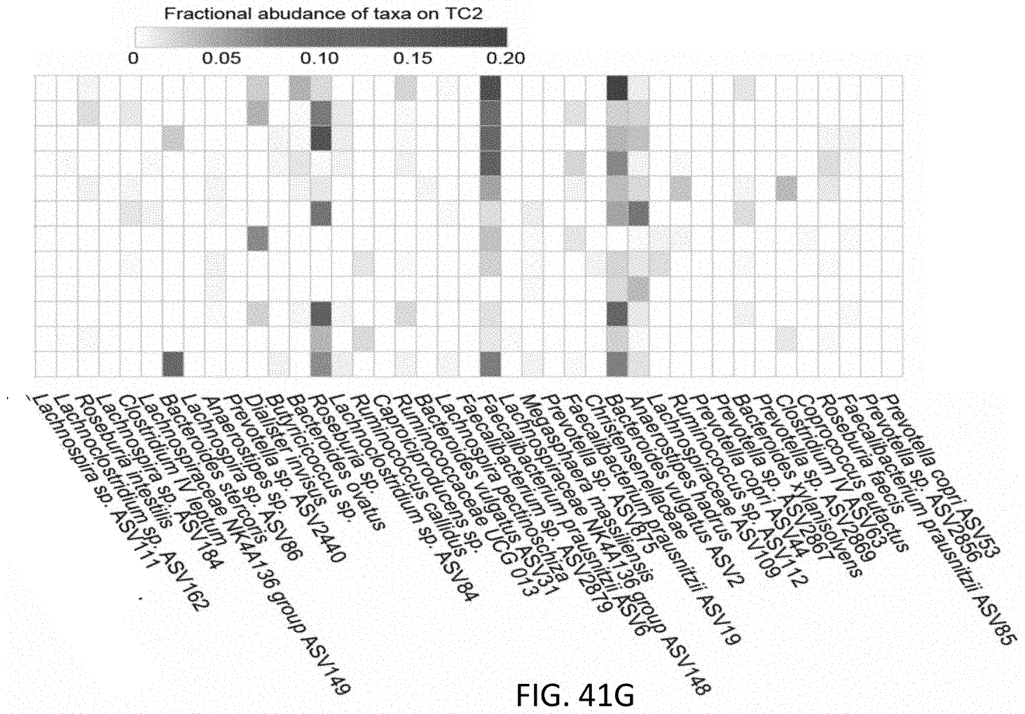

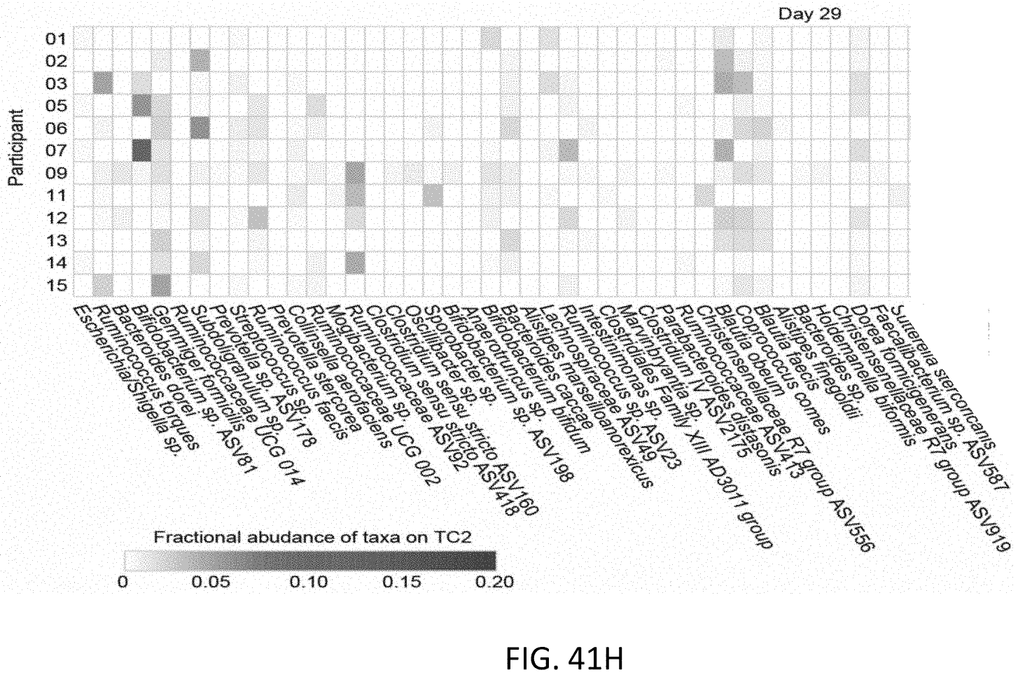

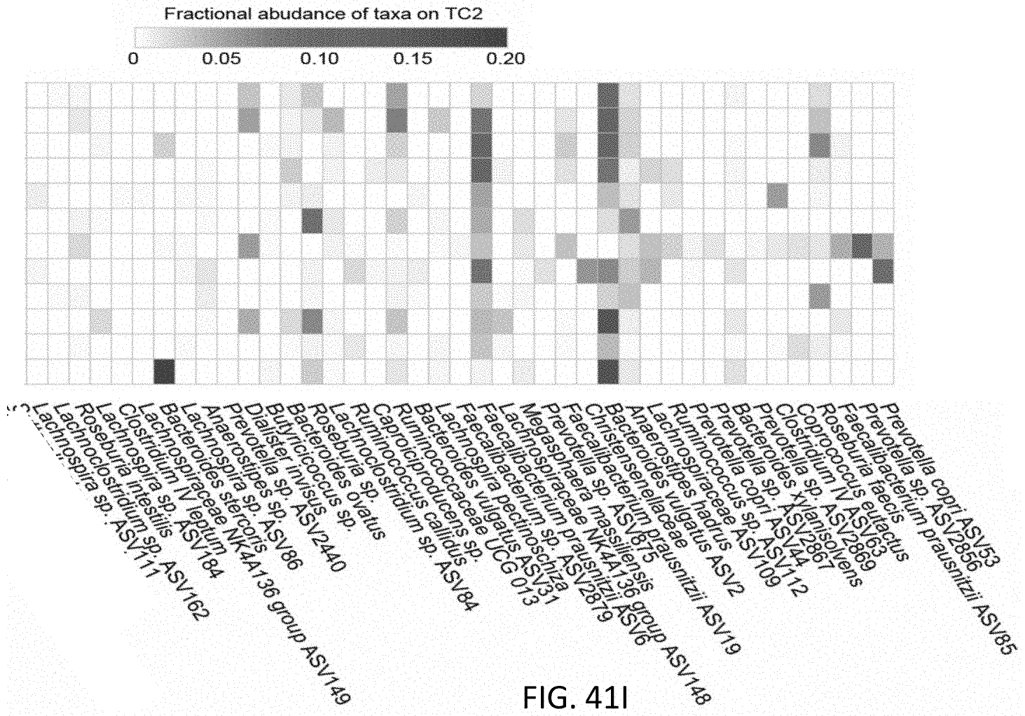

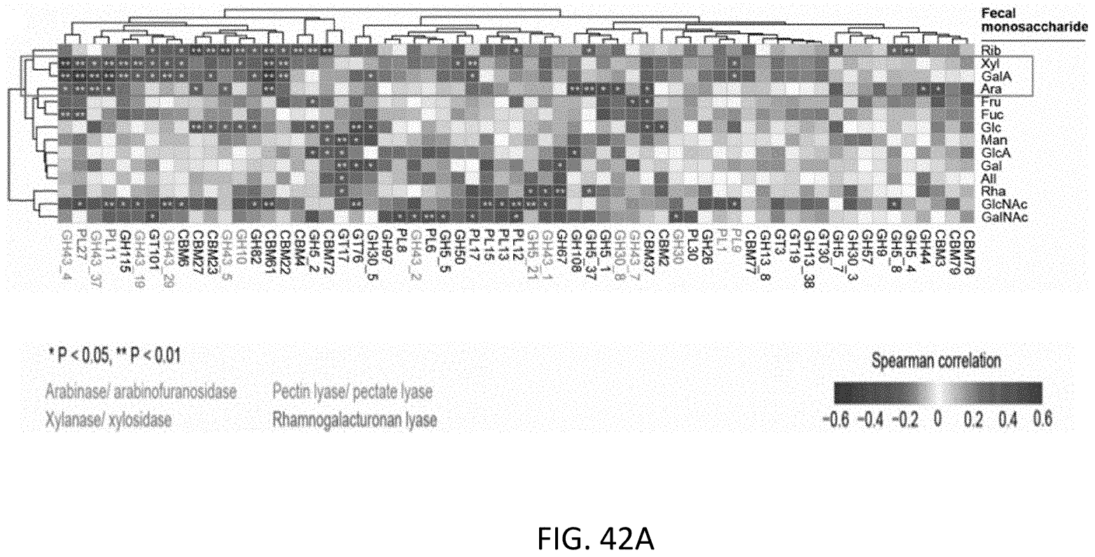

HO-SVD of the representation of CAZymes and mcSEED metabolic pathway components was performed to characterize the response of each subject (FIG. 31A,C,F,G, FIG. 40, FIG. 41. Two tensors were created where rows were subjects, columns were microbiome genes (either CAZymes or mcSEED metabolic pathways), and the third dimension was the three dietary conditions [HiSF-LoFV alone (pre-intervention phase), supplementation with the pea fiber snacks (3 snacks/day), and return to HiSF-LoFV (post-intervention phase) for a total of nine time points]. FIG. 31C demonstrates that gut microbiome CAZyme gene representation changed upon initiation of consumption of the pea fiber snack and moved towards the pre-treatment state when the intervention ceased. There was remarkable conservation of the treatment-discriminating CAZymes in these subjects and in pea fiber-treated gnotobiotic mice colonized with microbiota from distinct human donors; they include members of GH43 subfamily arabinofuranosidases and arabinanases (GH43_2, GH43_19), members of GH5 subfamily glucanases and glucosidases (GH5_1, GH5_4, GH5_5), pectin/pectate lyases (PL1, PL9), rhamnogalacturonan lyase (PL11), alginate lyase (PL6), and heparin lyase (PL13) [compare FIG. 31F,G (human) and FIG. 30E (mouse)]. The heatmaps in FIG. 31F,G, which plot the log₂ fold-change of CAZyme gene abundances in the human subjects' microbiomes relative to the time of initiation of pea fiber snack consumption (day 14), disclosed variations in a pea fiber CAZyme response across subjects. CAZymes were ranked based on their projections along TC4; only those within the top 20^{th} percentile of positive projections are shown. Hierarchical clustering of this discriminatory CAZyme dataset allowed us to group participants with similar microbiome responses to pea fiber snack consumption.

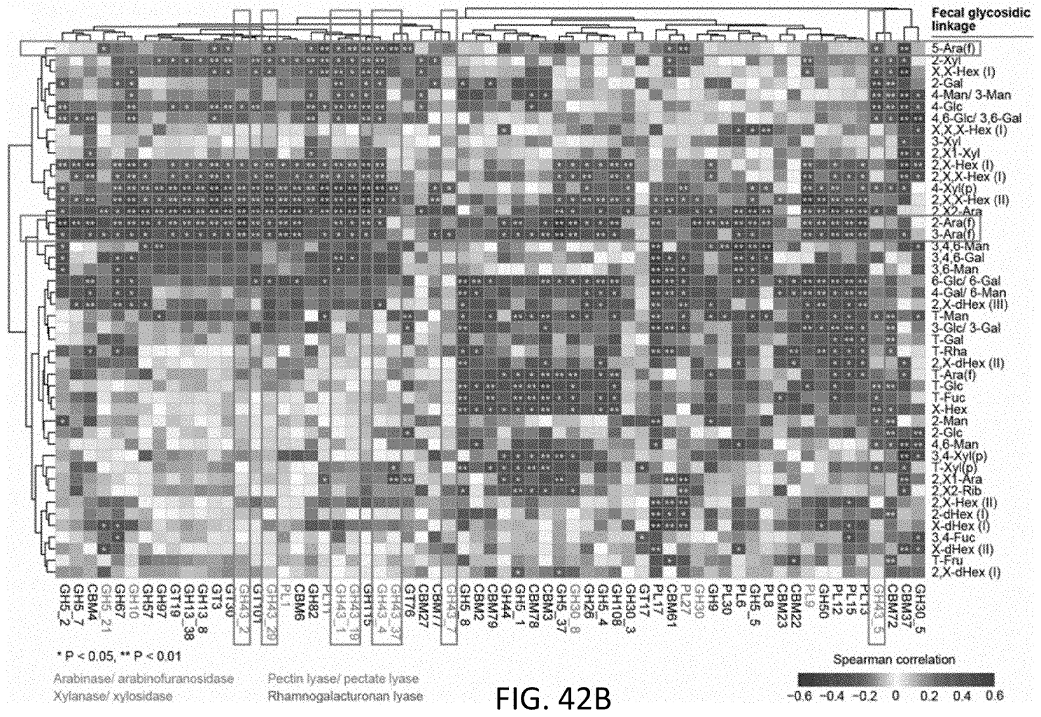

To examine the biotransformation of pea fiber by the participants' microbiota, we used liquid chromatography triple quadrupole mass spectrometry (LC-QQQ-MS) under dynamic multiple reaction monitoring (dMRM) to quantify the absolute concentrations of monosaccharides and the relative abundances of glycosidic linkages in fecal samples collected at the end of the pre-intervention phase (day 14), at peak dose of the fiber snack (days 29 and 35), and during the post-intervention period (days 45 and 49). A Spearman-rank cross-correlation analysis was performed between the log₂ fold-change of HO-SVD-defined discriminatory CAZyme gene abundances (top 20^{th} percentile; matched by time and subject) and the log₂ fold-change in levels of monosaccharides and glycosidic linkages normalized to day 14. Monosaccharides abundant in pea fiber (arabinose, xylose and galacturonic acid) significantly positively correlated with discriminatory CAZymes whose abundances increased during pea fiber supplementation (see areas in the green box in FIG. 42A). Cross-correlation analysis of fecal glycosidic linkages with discriminatory CAZyme gene abundances revealed a specific cleavage pattern of 1,2-arabinofuranose and 1,3-arabinofuranose linkages found in the branches of pea fiber arabinan (15) (FIG. 42B). Levels of these linkages, in addition to 3,4,6-galactose found in arabinogalactan branches, negatively correlated with discriminatory CAZymes that increased during pea fiber supplementation (including GH43_1, GH43_2, GH43_19, and GH43_29 which have known α-L-arabinofuranosidase activity). In contrast, levels of 1,5-arabinofuranose, a linkage primarily found in the backbone of pea fiber arabinan, positively correlated with discriminatory CAZymes that increased during pea fiber supplementation (FIG. 42B). Data suggest that the microbiomes of these participants are equipped with CAZymes capable of recognizing and cleaving multiple branches of pea fiber arabinan, leaving the backbone (1,5-arabinofuranose) to concentrate in feces.

TABLE 21A
Nutritional composition of fiber snack prototypes for human studies

| | | | Extruded snack pillows | | Extruded snack pillows with pea fiber + | |
|---|---|---|---|---|---|---|
| | Extruded pea fiber bars | | with pea fiber + inulin | | inulin + orange fiber + barley bran | |
| | Per | Per portion | Per | Per portion | Per | Per portion |
| Nutritional values | 100 g | (35 g) | 100 g | (30 g) | 100 g | (30 g) |
| Energy (kcal) | 370.0 | 130.0 | 304.0 | 91.0 | 311.0 | 93.0 |
| Protein (g) | 9.0 | 3.2 | 3.0 | 0.9 | 3.2 | 1.0 |
| Lipid (g) | 10.0 | 3.5 | 5.9 | 1.8 | 6.4 | 1.9 |
| Total carbohydrate (g) | 59.0 | 20.7 | 83.8 | 25.1 | 83.6 | 25.1 |
| Sugars (g) | 20.0 | 7.0 | 5.0 | 1.5 | 4.5 | 1.3 |
| Starch (g) | 16.0 | 5.6 | 48.1 | 14.4 | 47.0 | 14.0 |
| Total dietary fibers (g) | 23.0 | 8.1 | 33.6 | 10.1 | 34.9 | 10.5 |
| % of fiber on the snack | | | | | | |
| Pea fiber (extruded) | 100% | 100% | 64% | 64% | 33% | 33% |
| Inulin (HMW) | — | — | 36% | 36% | 36% | 36% |
| Orange fiber (extruded) | — | — | — | — | 11% | 11% |
| Barley bran | — | — | — | — | 20% | 20% |

HMW = high-molecular weight

TABLE 21B
Nutritional composition of HiSF-LoFV meals for human studies.

| | | Energy | Protein | Lipid | Carbohydrate | Fiber |
|---|---|---|---|---|---|---|
| Meal option | | (kcal) | (%) | (%) | (%) | (g) |
| Breakfast | Three meat biscuit pocket | 565.0 | 19.7 | 44.7 | 35.6 | 1.6 |
| | Dreamsicle ™ smoothie | 570.0 | 15.8 | 44.5 | 39.6 | 1.2 |
| | Pancake with sausage & bacon | 569.0 | 15.0 | 45.3 | 39.8 | 0.6 |
| | Chips Ahoy! smoothie | 571.0 | 15.6 | 44.0 | 40.5 | 1.3 |
| | Biscuit & gravy | 571.0 | 15.8 | 44.7 | 39.7 | 1.4 |
| | French toast with sausage & bacon | 571.0 | 15.0 | 44.9 | 40.1 | 1.2 |
| | Ham & cheese bagel | 570.0 | 20.2 | 44.2 | 35.5 | 1.4 |
| | Average per breakfast meal | 570.0 | 16.7 | 44.6 | 38.7 | 1.2 |
| Lunch/dinner | Toasted cheese and bacon sandwich | 570.0 | 19.7 | 45.0 | 35.5 | 1.4 |
| | (with soda) | | | | | |
| | Sloppy Joe on bun (with soda) | 570.0 | 20.0 | 40.5 | 39.6 | 1.1 |
| | Roast beef & salami sammies (with | 570.0 | 20.0 | 40.6 | 39.7 | 1.5 |
| | soda) | | | | | |
| | Cheeseburger sliders (with soda) | 570.0 | 19.3 | 40.6 | 40.2 | 1.5 |
| | Bacon mac & cheese (with soda) | 571.0 | 19.5 | 44.4 | 36.3 | 1.4 |
| | Bacon Cheddar BBQ hotdog (with | 570.0 | 15.2 | 45.8 | 39.1 | 1.0 |
| | soda) | | | | | |
| | Spaghetti with meat sauce (with soda) | 571.0 | 19.9 | 42.9 | 37.5 | 1.5 |
| | Quesadilla (with soda) | 570.0 | 18.9 | 42.9 | 38.4 | 1.5 |
| | Pig in a blanket (with soda) | 568.0 | 14.9 | 45.0 | 40.3 | 1.1 |
| | Pepperoni pizza (with soda) | 573.0 | 15.0 | 45.4 | 39.9 | 1.3 |
| | Nachos (with soda) | 570.0 | 17.7 | 44.3 | 38.2 | 1.5 |
| | Hamburger & sausage pizza (with | 570.0 | 16.9 | 43.8 | 39.6 | 1.3 |
| | soda) | | | | | |
| | Ham & cheese rollup with potato chips | 569.0 | 19.8 | 42.7 | 37.8 | 1.5 |
| | (with soda) | | | | | |
| | Chili mac (with soda) | 569.0 | 17.2 | 42.9 | 40.0 | 1.3 |
| | Average per lunch/ dinner meal | 570.0 | 18.1 | 43.3 | 38.7 | 1.3 |
| Snacks | Chocolate fudge bite | 100.0 | 16.2 | 44.2 | 39.6 | 0.3 |
| | Oreo ® cream cheese bite | 100.0 | 15.9 | 44.5 | 39.7 | 0.2 |
| | Saltines with Easy Cheese ™ | 101.0 | 19.0 | 43.9 | 37.1 | 0.3 |
| | Frozen orange soda smoothie | 100.0 | 16.1 | 44.3 | 16.1 | 0.1 |
| | Hard boiled egg & sour patch kids | 103.0 | 23.4 | 40.9 | 35.5 | 0 |
| | snack pack | | | | | |
| | String cheese & M&Ms ® | 100.0 | 15.4 | 47.6 | 37.1 | 0.3 |
| | Chocolate chip cookie bites & milk | 100.0 | 15.5 | 44.3 | 39.9 | 0.2 |
| | Average per snack | 101.0 | 17.4 | 44.2 | 35.0 | 0.2 |
| Fiber bar | Peanut butter rice krispies | 130.0 | 8.6 | 22.5 | 68.8 | 0.9 |
| substitute | | | | | | |

TABLE 22
Effects of consumption of the pea fiber snack prototype on clinical meta-data values.

| | | | | | FDR-adjusted P value (least- | | |
|---|---|---|---|---|---|---|---|
| | | | | P value | squares means of linear | | |
| | | | | (ANOVA of | mixed-effects model) | | |
| | Mean ± standard deviation | | | linear mixed- | Day 14 | Day 14 | Day 35 |
| Parameter | Day 14 | Day 35 | Day 49 | effects model) | vs 35 | vs 49 | vs 49 |
| BMI (kg/m²) | 30.2 ± 3.3 | 30.3 ± 3.2 | 31.2 ± 5.1 | 0.365 | 0.882 | 0.378 | 0.378 |
| Fasting insulin (pmol/L) | 17.5 ± 7.6 | 17.4 ± 9.4 | 14.7 ± 8.2 | 0.175 | 0.951 | 0.169 | 0.169 |
| Fasting glucose (mg/dL) | 100.7 ± 12.9 | 106.8 ± 12.8 | 103.9 ± 13.7 | 0.066 | 0.064 | 0.261 | 0.261 |
| HOMA-IR | 4.5 ± 2.3 | 4.8 ± 3.1 | 4.0 ± 2.7 | 0.271 | 0.498 | 0.498 | 0.335 |
| Triglyceride (mg/dL) | 122.7 ± 62.6 | 126.8 ± 81.7 | 125.3 ± 84.4 | 0.927 | 0.891 | 0.891 | 0.891 |
| Total cholesterol (mg/dL) | 194.1 ± 26.2 | 190.8 ± 33.6 | 185.8 ± 30.5 | 0.243 | 0.509 | 0.297 | 0.457 |
| HDL cholesterol (mg/dL) | 47.9 ± 10.8 | 47.2 ± 9.8 | 46.4 ± 9.3 | 0.656 | 0.647 | 0.647 | 0.647 |
| LDL cholesterol (mg/dL) | 121.6 ± 21.8 | 118.3 ± 29.8 | 114.3 ± 25.7 | 0.264 | 0.453 | 0.321 | 0.453 |
| Hemoglobin (g/dL) | 14.2 ± 1.2 | 14.0 ± 1.4 | 13.8 ± 1.5 | 0.032 | 0.275 | 0.029 | 0.152 |
| Hematocrit (%) | 42.4 ± 3.0 | 42.2 ± 3.6 | 41.5 ± 4.1 | 0.106 | 0.739 | 0.143 | 0.143 |
| Red blood count (×10¹²/L) | 5.0 ± 0.6 | 4.9 ± 0.5 | 4.8 ± 0.6 | 0.118 | 0.676 | 0.155 | 0.175 |
| Platelet count (×10⁹/L) | 267.7 ± 79.4 | 256.6 ± 74.8 | 257.7 ± 67.7 | 0.128 | 0.144 | 0.144 | 0.852 |
| Mean platelet volume (fL) | 8.7 ± 1.0 | 8.8 ± 0.8 | 8.8 ± 0.9 | 0.268 | 0.269 | 0.269 | 0.909 |
| White blood count (×10⁹/L) | 5.8 ± 1.3 | 5.9 ± 1.4 | 5.7 ± 1.3 | 0.870 | 0.917 | 0.917 | 0.917 |
| Neutrophil (%) | 53.2 ± 9.4 | 55.1 ± 7.7 | 53.7 ± 7.8 | 0.488 | 0.589 | 0.766 | 0.589 |
| Lymphocyte (%) | 34.9 ± 7.9 | 33.3 ± 6.5 | 34.9 ± 6.9 | 0.432 | 0.398 | 1.000 | 0.398 |
| Monocyte (%) | 8.5 ± 2.6 | 8.3 ± 2.1 | 7.7 ± 1.5 | 0.240 | 0.652 | 0.317 | 0.347 |
| Eosinophil (%) | 2.6 ± 1.3 | 2.7 ± 1.4 | 2.9 ± 1.5 | 0.542 | 0.668 | 0.668 | 0.668 |
| Basophil (%) | 0.8 ± 0.3 | 0.7 ± 0.3 | 0.8 ± 0.3 | 0.183 | 0.242 | 0.721 | 0.242 |
| Absolute neutrophil (×10⁹/L) | 3.1 ± 1.1 | 3.3 ± 1.1 | 3.1 ± 0.9 | 0.670 | 0.717 | 0.915 | 0.717 |
| Absolute lymphocyte (×10⁹/L) | 3.4 ± 4.9 | 1.9 ± 0.5 | 2.0 ± 0.5 | 0.373 | 0.353 | 0.353 | 0.978 |
| Absolute monocyte (×10⁹/L) | 0.5 ± 0.1 | 0.5 ± 0.1 | 0.4 ± 0.1 | 0.177 | 0.532 | 0.327 | 0.211 |
| Absolute eosinophil (×10⁹/L) | 0.2 ± 0.1 | 0.1 ± 0.1 | 0.2 ± 0.3 | 0.446 | 0.897 | 0.458 | 0.458 |
| Absolute basophil (×10⁹/L) | 0.0 ± 0.1 | 0.0 ± 0.1 | 0.0 ± 0.1 | 0.468 | 0.434 | 0.434 | 1.000 |
| Total protein (g/dL) | 7.5 ± 0.6 | 7.5 ± 0.5 | 7.4 ± 0.6 | 0.566 | 0.879 | 0.607 | 0.607 |
| Albumin (g/L) | 4.5 ± 0.3 | 4.4 ± 0.2 | 4.4 ± 0.2 | 0.105 | 0.270 | 0.112 | 0.415 |
| Calcium (mmol/L) | 9.3 ± 0.4 | 9.2 ± 0.4 | 9.2 ± 0.4 | 0.263 | 0.327 | 0.473 | 0.527 |
| Total bilirubin (μmol/L) | 0.5 ± 0.3 | 0.5 ± 0.3 | 0.5 ± 0.3 | 0.813 | 0.885 | 0.885 | 0.885 |
| SGOT (AST) (U/L) | 19.5 ± 7.5 | 20.3 ± 7.4 | 20.8 ± 5.7 | 0.516 | 0.679 | 0.679 | 0.706 |
| SGPT (ALT) (U/L) | 21.6 ± 18.7 | 23.4 ± 17.6 | 20.8 ± 12.8 | 0.493 | 0.638 | 0.716 | 0.638 |
| Alk. phos, total (IU/L) | 64.3 ± 16.0 | 64.8 ± 16.0 | 62.3 ± 14.5 | 0.075 | 0.700 | 0.112 | 0.102 |
| Urea nitrogen (mg/dL) | 15.8 ± 3.3 | 14.0 ± 4.2 | 14.7 ± 3.9 | 0.009 | 0.007 | 0.069 | 0.206 |
| Creatinine (mg/dL) | 1.0 ± 0.2 | 1.0 ± 0.1 | 1.0 ± 0.2 | 0.691 | 0.705 | 0.705 | 0.705 |
| Sodium (mmol/L) | 138.8 ± 2.1 | 138.6 ± 1.6 | 138.2 ± 1.6 | 0.306 | 0.563 | 0.395 | 0.508 |
| Potassium (mmol/L) | 4.2 ± 0.3 | 4.2 ± 0.3 | 4.1 ± 0.3 | 0.067 | 0.866 | 0.078 | 0.078 |
| Chloride (mEq/L) | 102.4 ± 2.4 | 102.8 ± 2.1 | 102.8 ± 2.4 | 0.640 | 0.717 | 0.717 | 0.858 |
| CO₂ content (mEq/L) | 24.8 ± 1.8 | 24.3 ± 2.3 | 23.6 ± 2.0 | 0.027 | 0.258 | 0.025 | 0.144 |

### Example 15. Effects of Snack Food Containing Mixtures of Two and Four Fibers in Overweight or Obese Adults

To determine whether combining pea fiber with the other fibers characterized in our gnotobiotic mouse experiments would have greater effects on the microbiome and host than those obtained with pea fiber alone, we performed a second controlled diet study involving 14 people who were overweight or obese, nine of whom had also participated in the pea fiber study (see Table 29 for a description of subjects enrolled). Two multi-fiber snack prototypes were tested; one contained pea fiber and inulin (10.1 gram (g) fiber/30 g snack; 64% pea fiber and 36% inulin) and the other a combination of pea fiber, inulin, orange fiber and barley bran (10.5 g fiber/30 g snack; 33% pea fiber, 36% inulin, 11% A orange fiber, and 20% barley bran; Table 21A). Inulin, isolated from the root of chicory Cichorium intybus, is a beta-2-1-linked fructose polymer with limited degree of polymerization relative to many other dietary plant polysaccharides (20). In our previously published gnotobiotic mouse fiber screening experiment involving the 20-member consortium of cultured human gut bacterial strains (15), each 1% w/w increase in the amount of inulin added to the HiSF-LoFV diet resulted in a pronounced 4.5% increase in the relative abundance of another target Bacteroides, B. caccae, which possessed GH2 enzymes involved in beta-2-1-linked fructan metabolism as well as a GH91 inulin lyase (15).

The study design is summarized in FIG. 31B. On the first day, each participant provided a fecal sample produced while on their normal diet; they then followed a 48-day controlled diet protocol where their normal diet was replaced with the same HiSF-LoFV diet used for the pea fiber study (Table 21B). Subjects were provided with the pea fiber/inulin snack food prototype to supplement their HiSF-LoFV diet for 14 days starting on study day 12. The dosage escalation protocol consisted of one snack/day for one day (day 12, with lunch), followed by two snacks/day for an additional day (day 13; lunch and dinner), and three snacks a day for the following 12 days (breakfast, lunch and dinner). Participants continued on the HiSF-LoFV diet for 10 additional days after stopping consumption of the two-fiber snack food prototype and then started on the four-fiber blend for 14 days. The dosage escalation protocol for this phase consisted of one snack/day for one day (day 36), followed by two snacks/day for an additional day (day 37), and three snacks a day for the following 12 days (with total caloric intake maintained constant throughout). During the course of the study, no additional caloric adjustments had to be made in the amount of HiSF-LoFV diet consumed to maintain a constant body weight in all subjects. Blood (plasma) and fecal samples were collected at the time points shown in FIG. 31B; fecal samples were used to generate shotgun sequencing and V4-16S rDNA amplicon datasets.

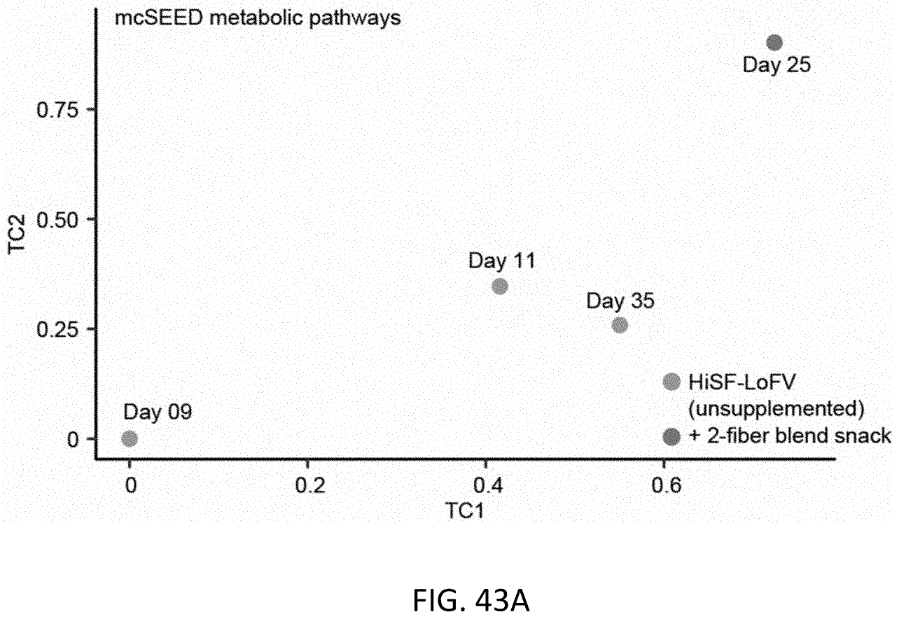

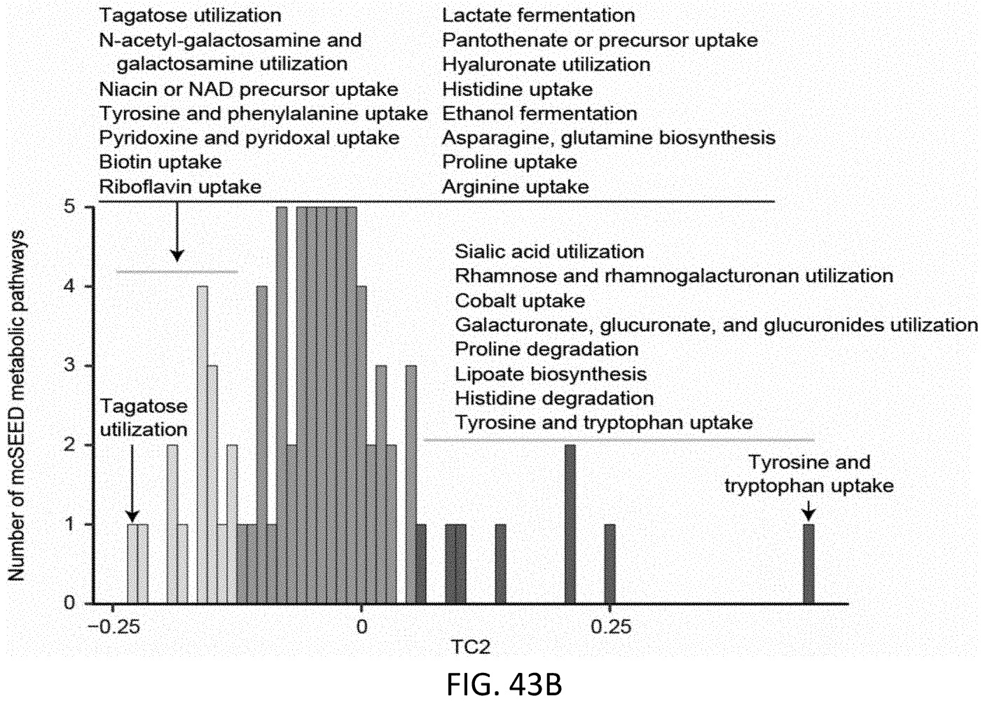

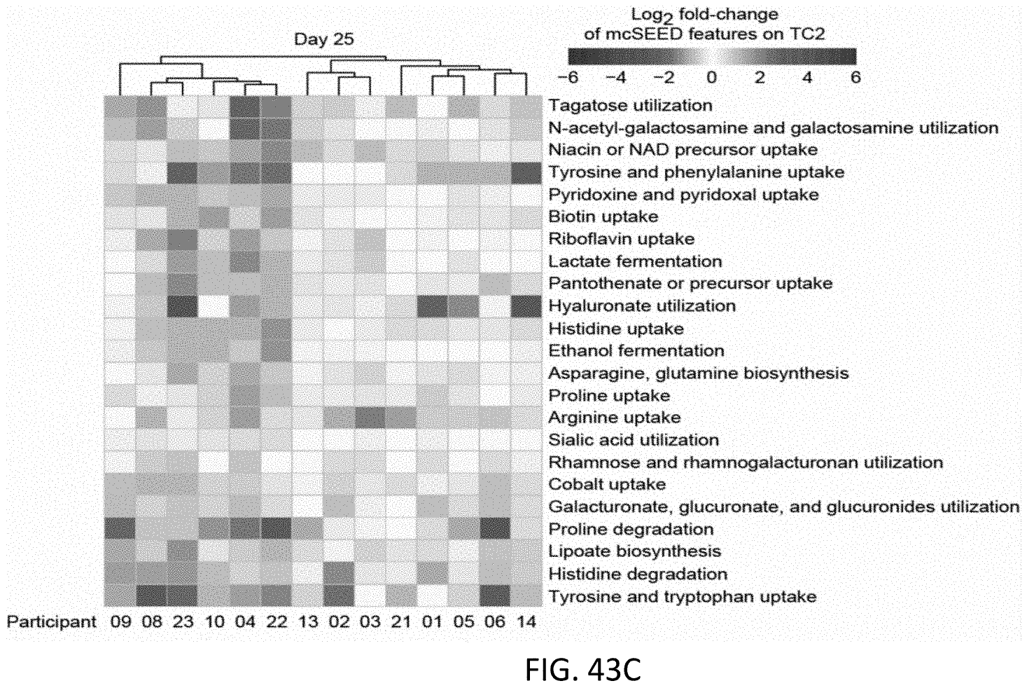

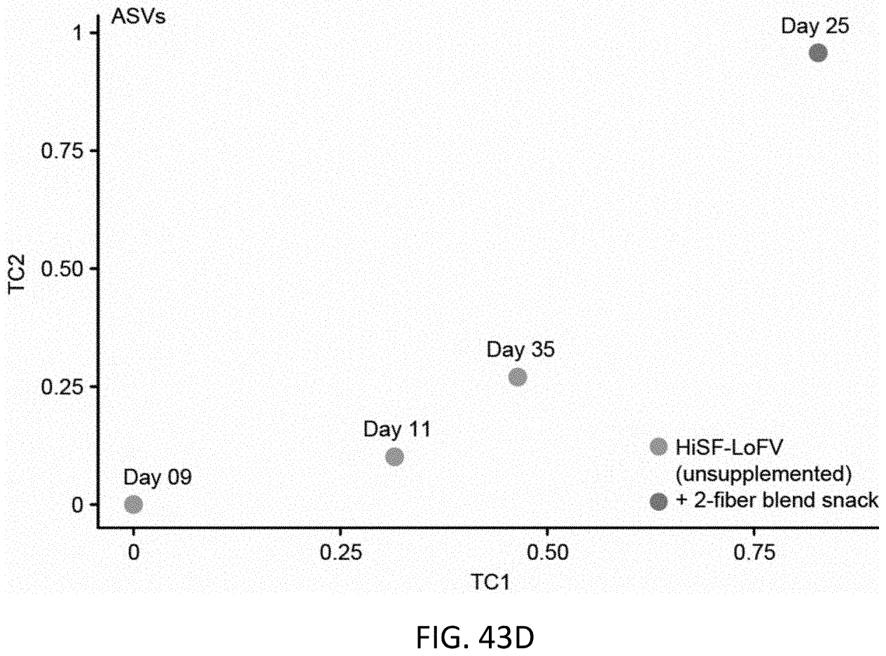

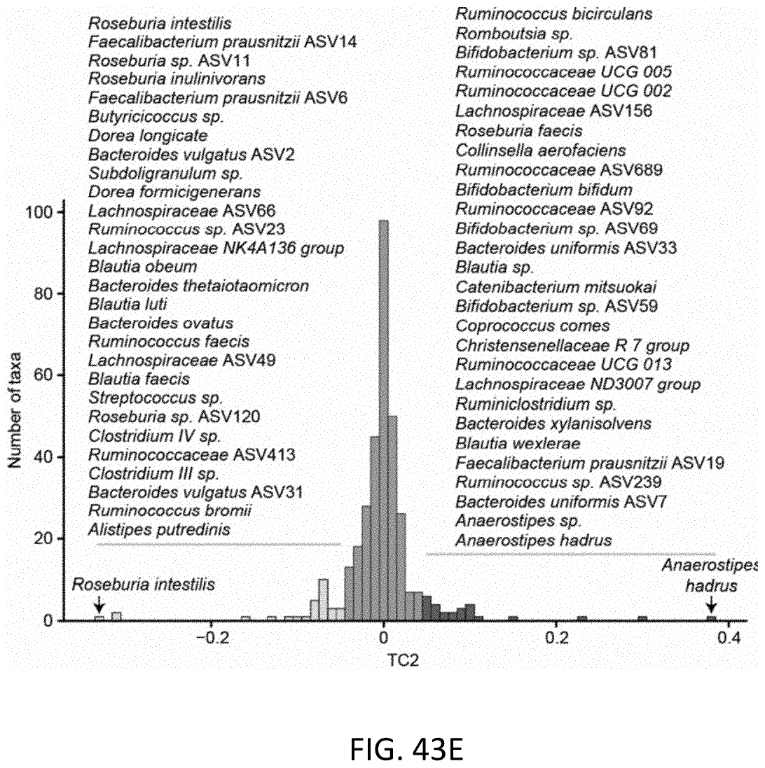

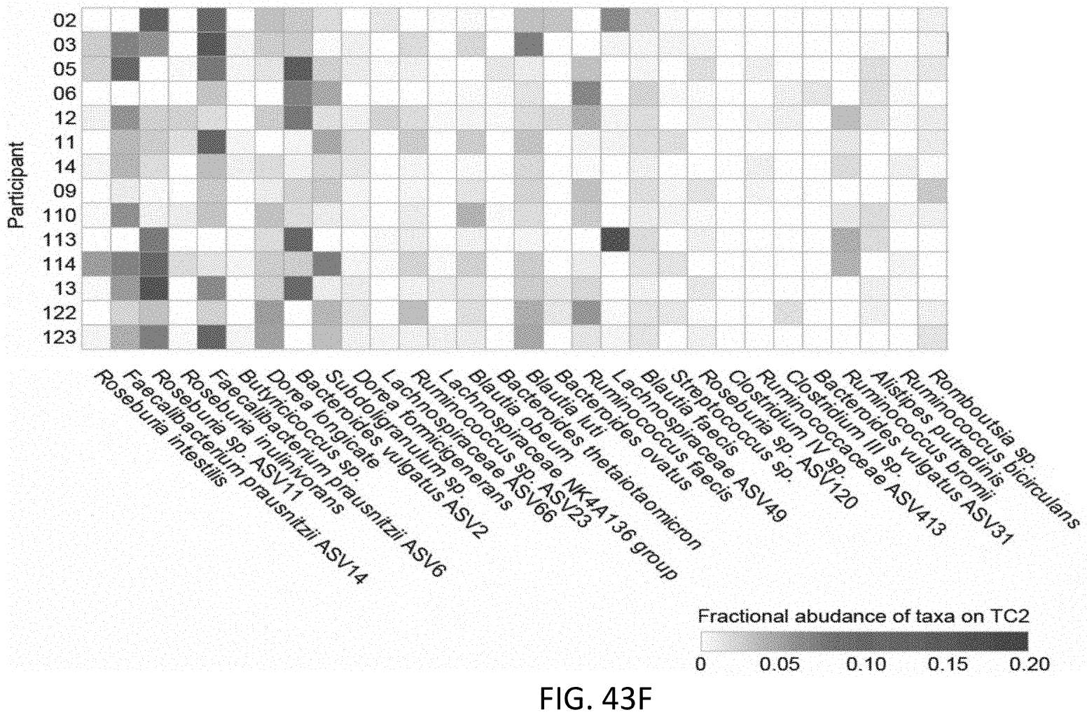

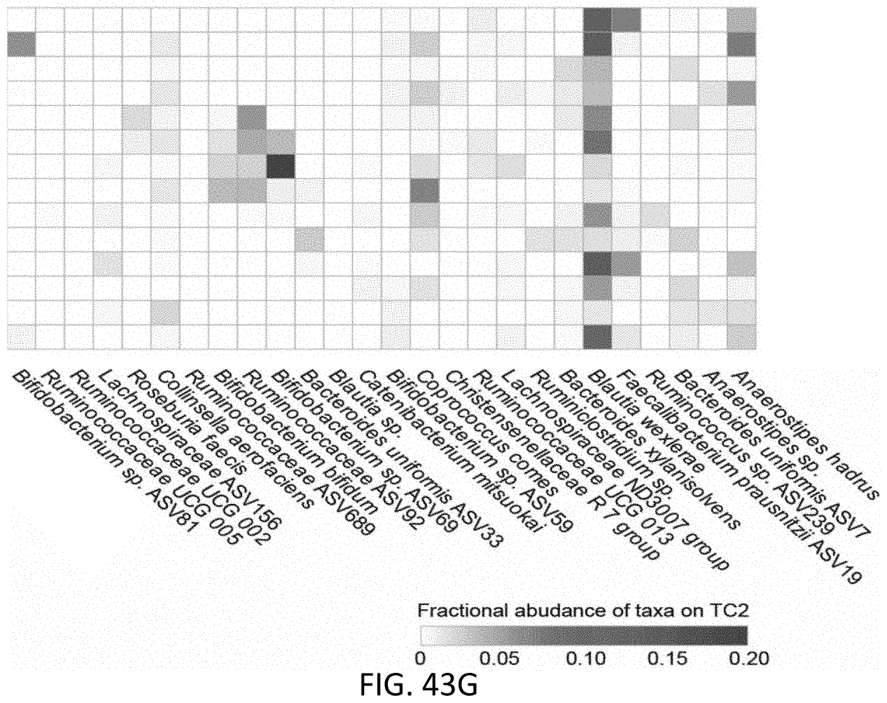

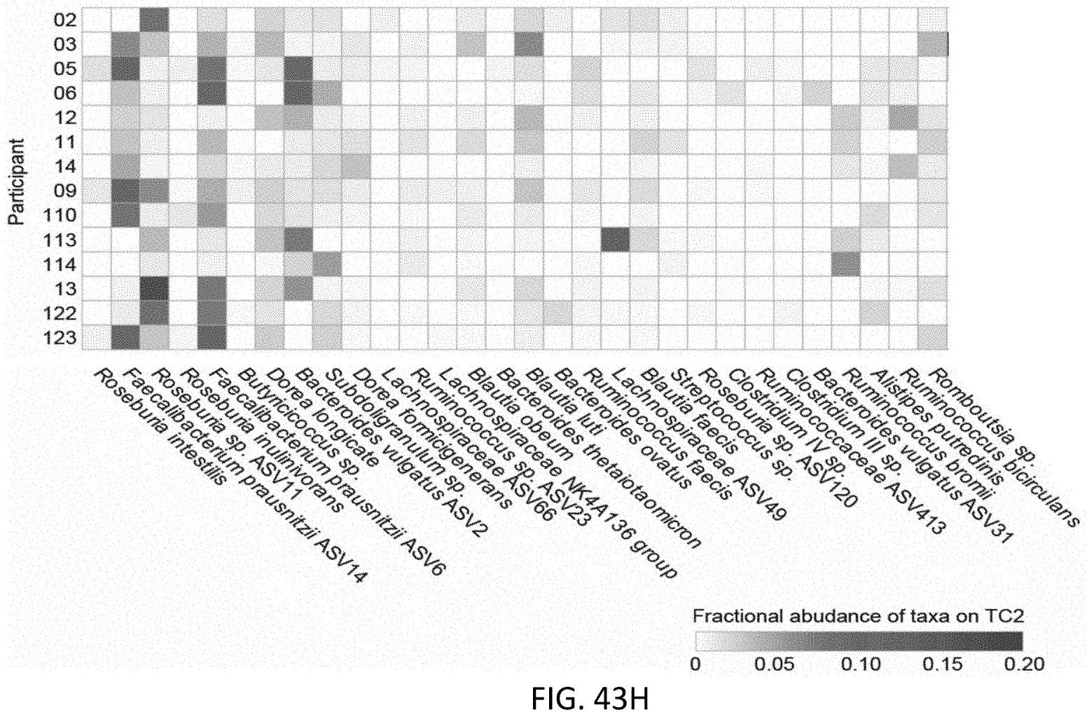

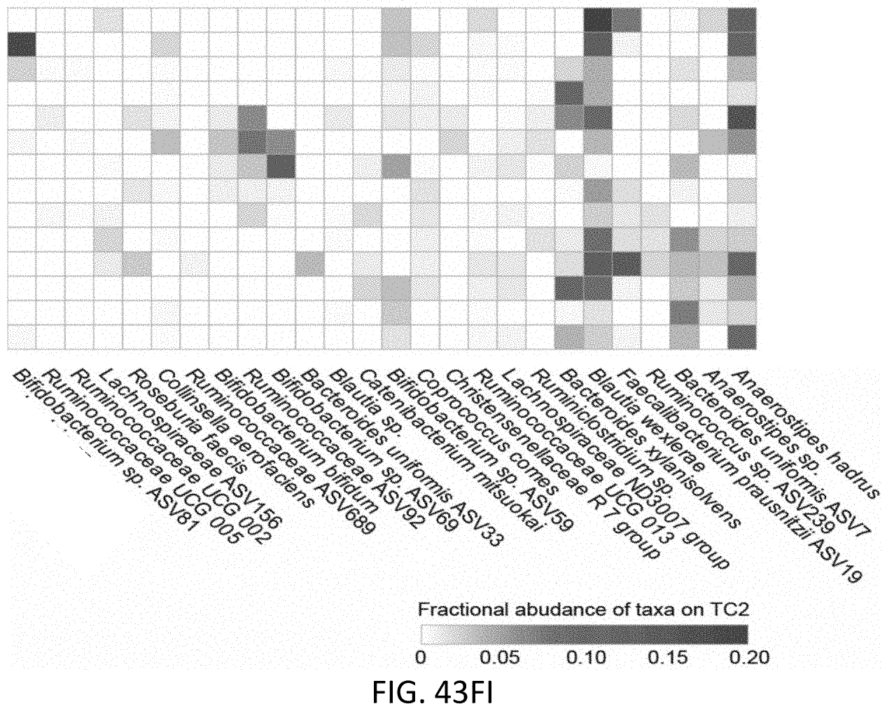

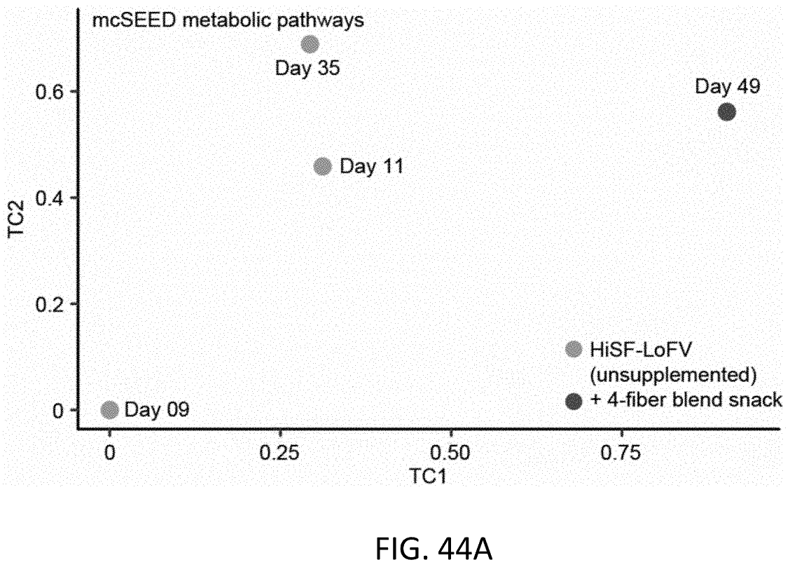

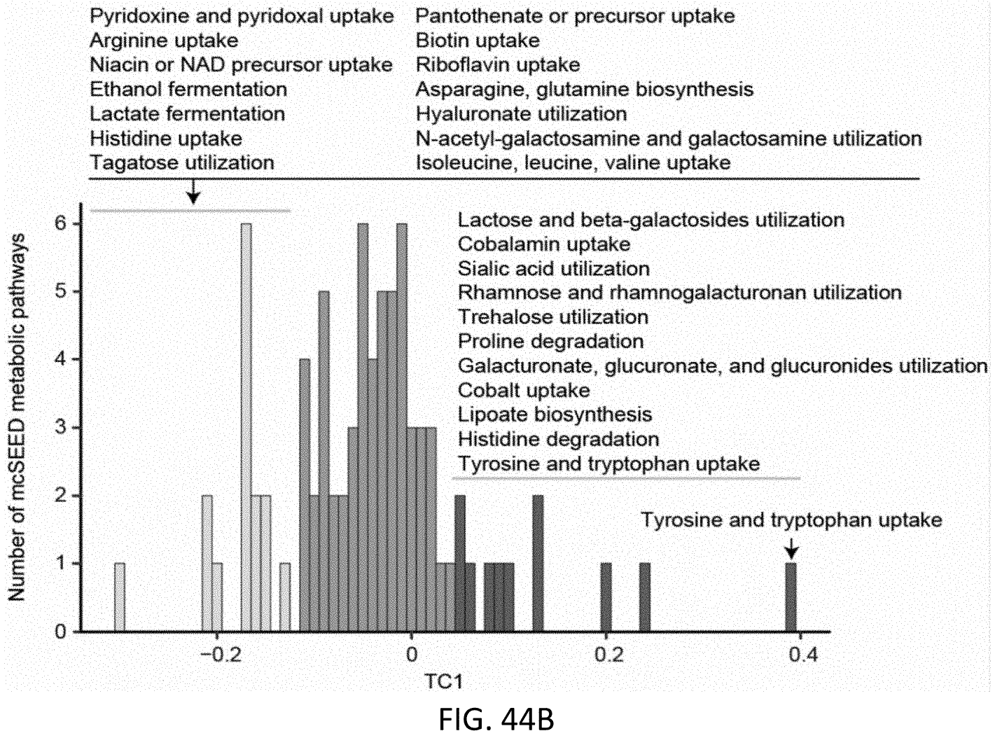

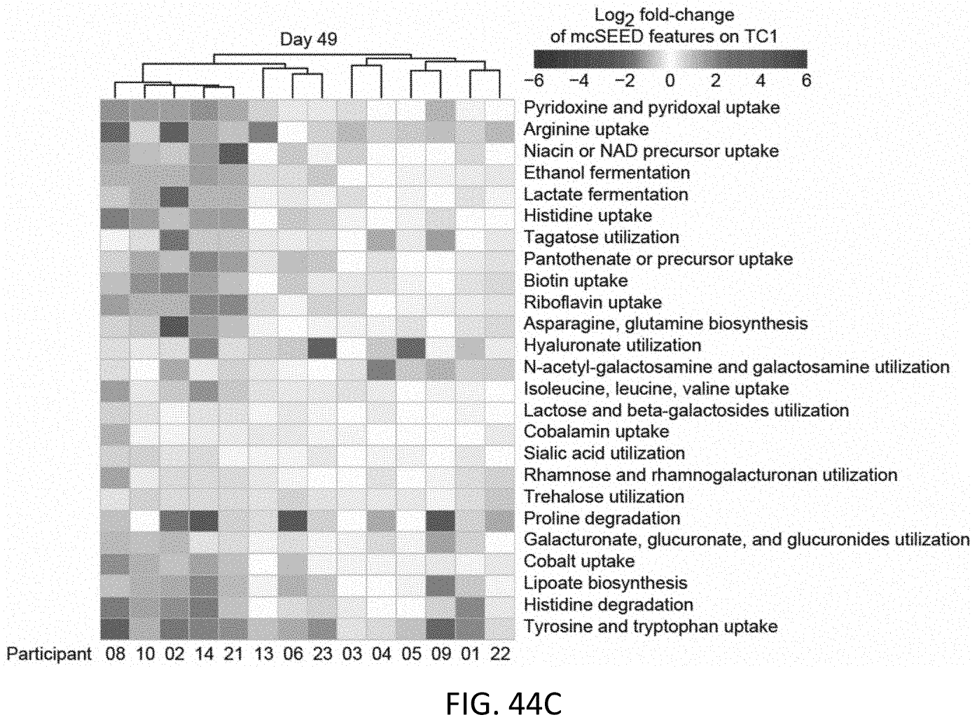

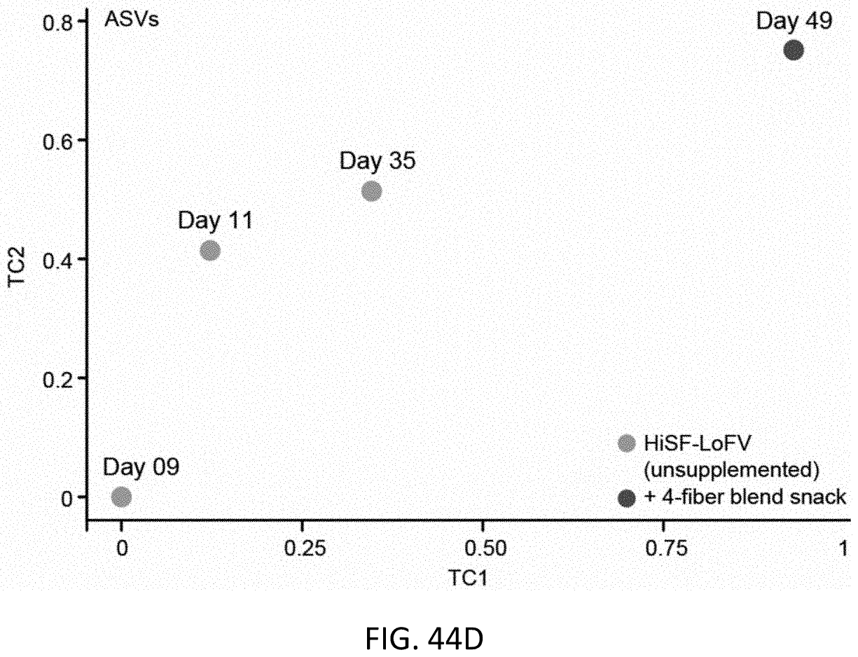

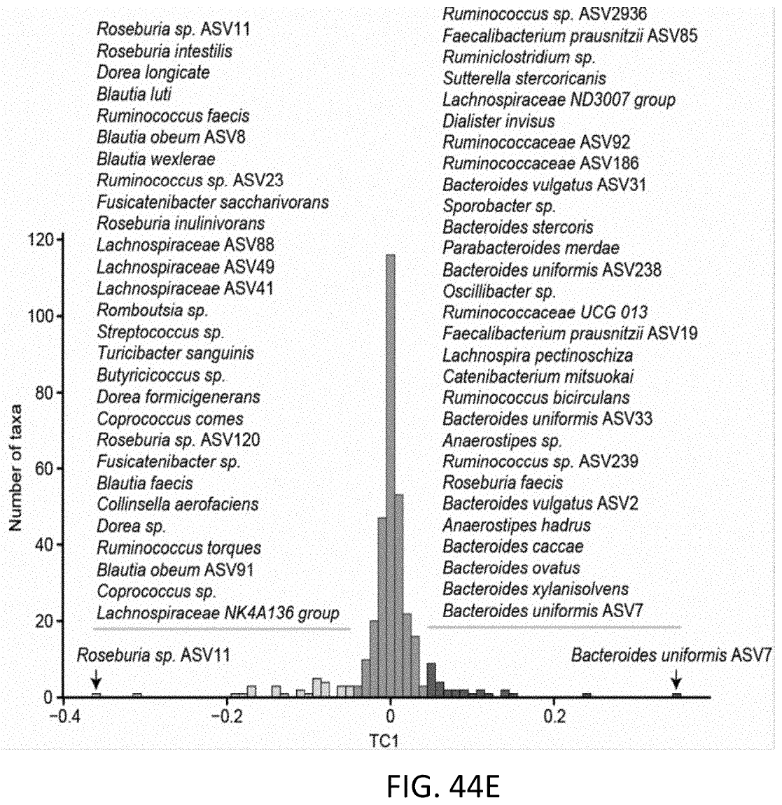

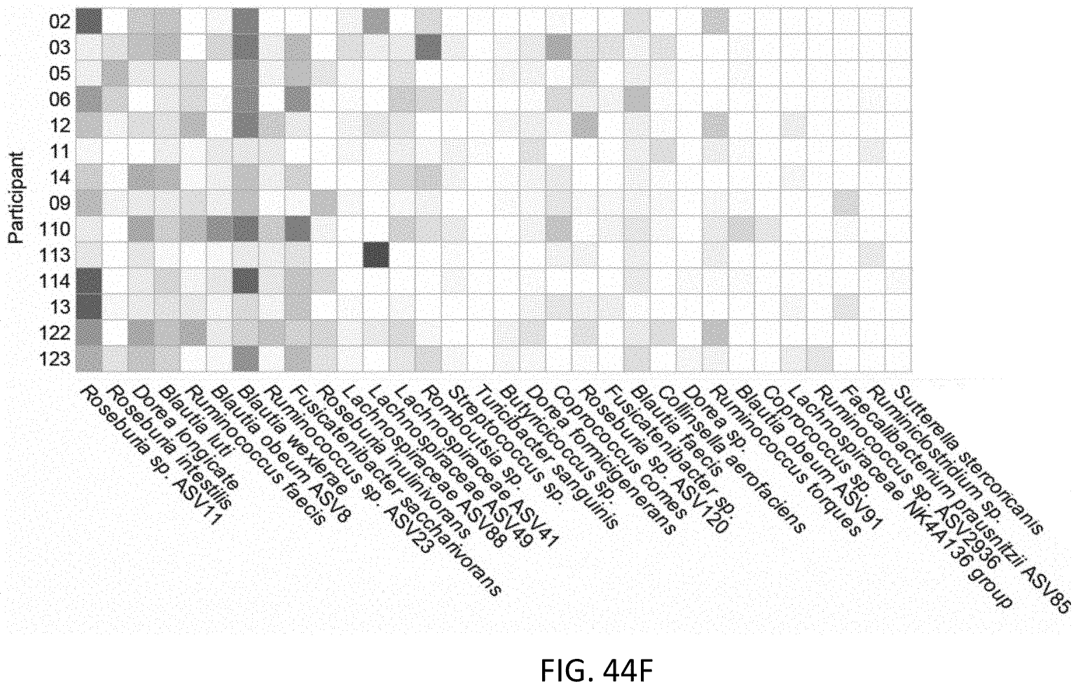

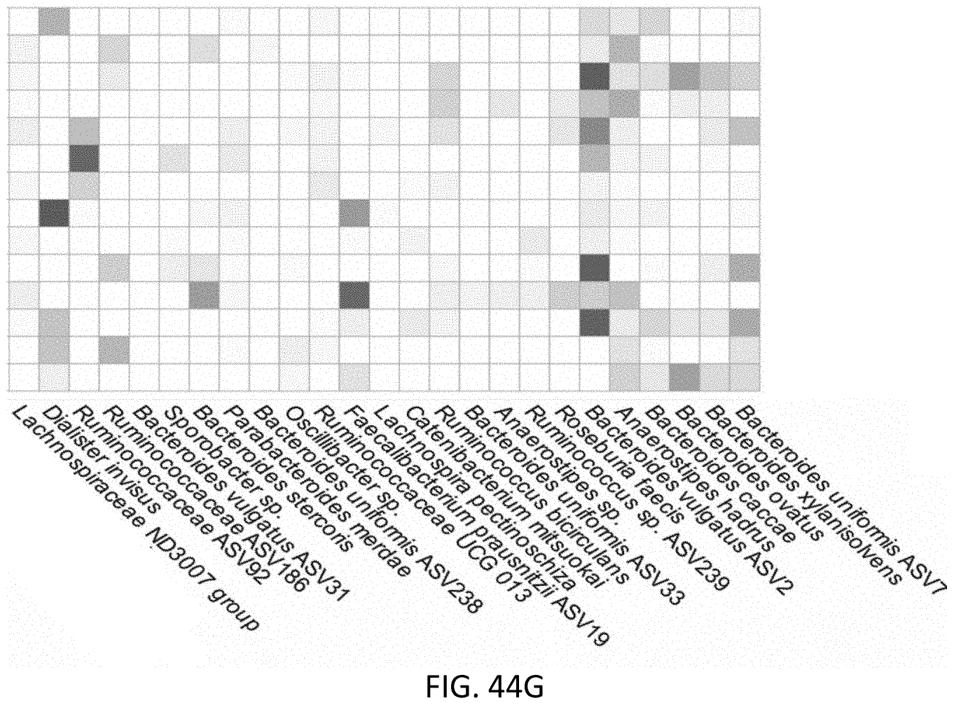

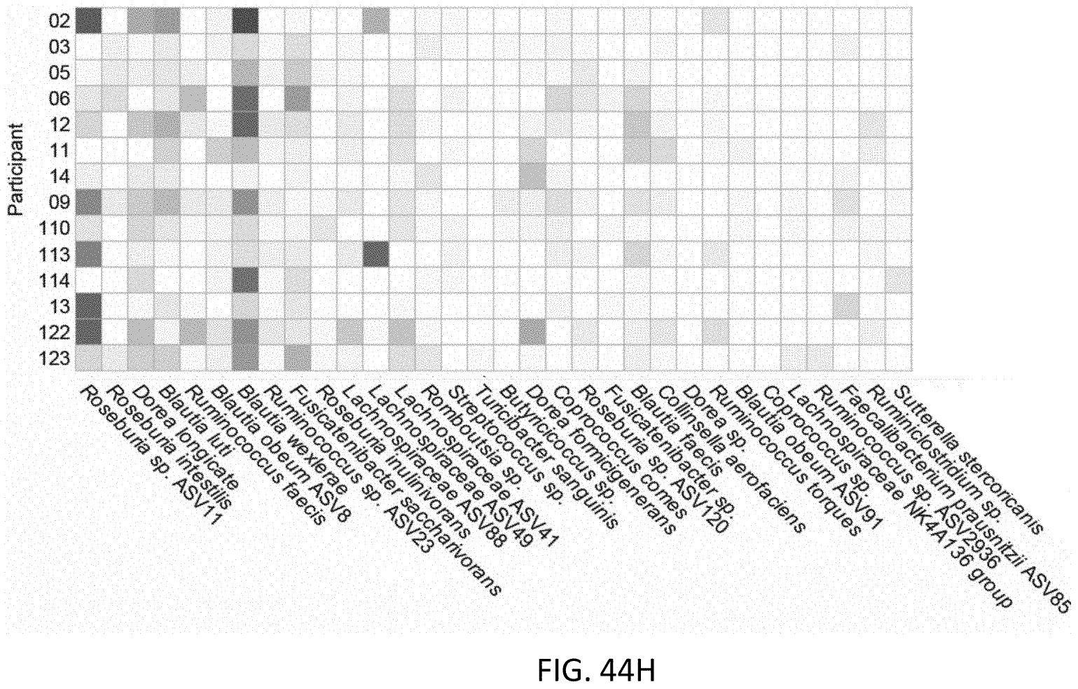

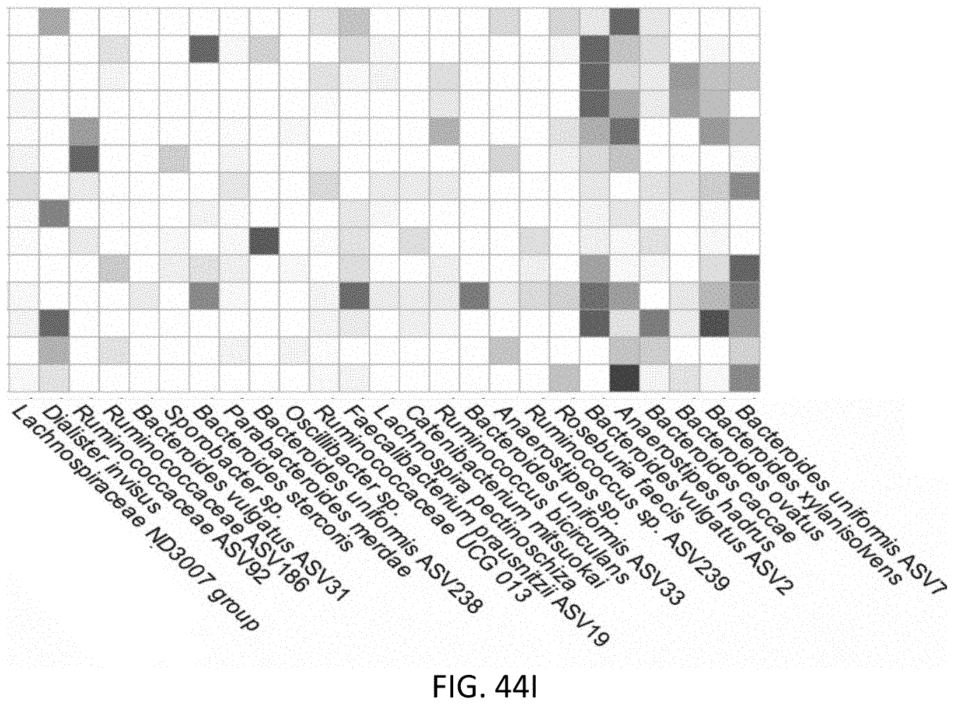

We constructed two tensors to distinguish the effects of each type of snack prototype on the CAZyme composition of participant microbiomes. In both tensors, rows represented subjects and columns were genes (log₂ fold-change of CAZymes normalized to day 9 when subjects were on the HiSF-LoFV base diet). The third dimension represented study days corresponding to the different diet conditions. The results of HO-SVD analyses are provided in FIG. 31, FIG. 40, FIG. 43 and FIG. 44.

Changes in microbiome CAZyme gene composition in response to the dietary interventions with both fiber blend preparations were represented by changes in projection along TC1 (FIG. 31D,E). The heatmaps in FIG. 31H-K display the log₂ fold-change in CAZyme gene abundances normalized to the last day of the pre-intervention phase (day 11). CAZymes were ranked based on their projections along TC1; only those within the top 20^{th} percentile of positive projections are shown. Increased representation of genes encoding arabinan-processing enzymes, including the arabinase/arabinofuranosidases GH43_1 and GH43_19, occurs with both the two- and four-fiber blends (FIG. 35G,H) as well as with the pea fiber alone formulation (FIG. 35H, I), whereas GH43, GH43_9, GH43_18, GH43_28, GH43_33, and GH43_34 CAZymes increased with the two- and four-fiber snack food prototypes (FIG. 35G,H). CAZymes that process galacturonic acid and xylose increased in response to all three fiber snacks including the xylanase/xylosidase GH30, pectin/pectate lyases PL1, PL9, and the rhamnogalacturonan lyase PL11 (FIG. 35F-H). The inulin-processing GH91 and inulin-binding protein CBM38 were part of the response to the four-fiber combination, with CBM38 but not GH91 increasing after exposure to the pea fiber plus inulin combination (FIG. 35G,H). While galacturonan and arabinan are abundant in pea fiber and orange fiber, beta-glucans and arabinoxylans are prominent in barley bran (15). The representation of rhamnogalacturonan lyase PL26, the α-galacturonidase GH138, the α-L-arabinofuranosidase GH43_16, and the endo-1,2-α-mannanase GH99 were only found to change after consumption of the four-fiber blend snack, as did the β-glucosidase GH116 (FIG. 35H), consistent with the targeting of beta-glucans that were distinctly represented in barley bran. Hierarchical clustering (Canberra distance) of changes in discriminatory CAZyme gene abundances provided an informative way to compare and group the microbiome responses of participants to each of the snack prototypes (FIG. 35F-H).

Comparable HO-SVD analyses of the effects of the two-fiber and four-fiber blends on mcSEED pathway and ASV composition are presented in FIG. 43 and FIG. 44. With both fiber blends, there was an increase in the representation of pathways for rhamnose and rhamnogalacturonan utilization, and galacturonate, glucuronate and glucuronides utilization (FIG. 43B,C; FIG. 44B,C). The four-fiber blend snack produced significant increases in the abundance of a broader range of targeted Bacteroides species, including B. vulgatus, B. uniformis, B. xylanisolvens, B. ovatus and B. caccae, than the two-fiber blend or pea fiber alone (FIG. 41E,F; FIG. 43E,F,G,H,I; FIG. 44E,F,G,H,I).

TABLE 23
Effects of consumption of the snack fiber prototypes on clinical meta-data values.
(A)

| | | | | | P value (ANOVA |
|---|---|---|---|---|---|
| | Mean ± standard deviation | | | | of linear mixed- |
| Parameter | Day 11 | Day 25 | Day 35 | Day 49 | effects model) |
| BMI (kg/m²) | 29.6 ± 3.6 | 29.6 ± 3.7 | 29.5 ± 3.8 | 29.5 ± 3.7 | 0.406 |
| Fasting insulin (pmol/L) | 12.1 ± 6.0 | 14.3 ± 8.0 | 14.7 ± 8.2 | 12.4 ± 5.9 | 0.050 |
| Fasting glucose (mg/dL) | 93.0 ± 6.9 | 95.1 ± 6.1 | 97.6 ± 8.5 | 96.9 ± 6.1 | 0.002 |
| HOMA-IR | 2.8 ± 1.5 | 3.4 ± 2.1 | 3.6 ± 2.3 | 3.0 ± 1.6 | 0.027 |
| Triglyceride (mg/dL) | 93.1 ± 40.6 | 96.8 ± 45.1 | 104.6 ± 46.4 | 96.3 ± 46.7 | 0.309 |
| Total cholesterol (mg/dL) | 188.9 ± 20.4 | 187.5 ± 26.2 | 187.9 ± 27.4 | 185.7 ± 29.4 | 0.913 |
| HDL cholesterol (mg/dL) | 52.3 ± 9.9 | 51.6 ± 9.8 | 51.7 ± 9.9 | 51.4 ± 9.4 | 0.895 |
| LDL cholesterol (mg/dL) | 117.8 ± 19.8 | 116.5 ± 24.2 | 115.2 ± 27.0 | 115.0 ± 27.1 | 0.878 |
| Hemoglobin (g/dL) | 13.7 ± 0.8 | 13.6 ± 0.8 | 13.8 ± 0.9 | 13.7 ± 1.1 | 0.724 |
| Hematocrit (%) | 41.0 ± 2.2 | 40.6 ± 1.9 | 40.9 ± 2.3 | 41.1 ± 2.9 | 0.753 |
| Red blood count (×10¹²/L) | 4.7 ± 0.6 | 4.7 ± 0.5 | 4.7 ± 0.6 | 4.7 ± 0.6 | 0.625 |
| Platelet count (×10⁹/L) | 260.7 ± 70.6 | 259.9 ± 66.1 | 258.1 ± 70.7 | 263.4 ± 58.0 | 0.780 |
| Mean platelet volume (fL) | 8.9 ± 0.9 | 8.9 ± 0.8 | 8.9 ± 0.9 | 8.9 ± 0.8 | 0.962 |
| White blood count (×10⁹/L) | 5.4 ± 1.0 | 5.2 ± 1.1 | 5.3 ± 1.2 | 5.5 ± 1.4 | 0.707 |
| Neutrophil (%) | 50.4 ± 7.4 | 50.4 ± 7.2 | 51.7 ± 8.9 | 53.1 ± 8.1 | 0.218 |
| Lymphocyte (%) | 37.9 ± 7.0 | 38.3 ± 6.9 | 36.8 ± 7.0 | 35.7 ± 7.7 | 0.290 |
| Monocyte (%) | 8.3 ± 1.8 | 7.9 ± 1.8 | 8.1 ± 1.9 | 7.8 ± 1.7 | 0.413 |
| Eosinophil (%) | 2.6 ± 1.8 | 2.5 ± 1.5 | 2.4 ± 1.4 | 2.5 ± 1.9 | 0.929 |
| Basophil (%) | 0.9 ± 0.6 | 0.9 ± 0.3 | 1.0 ± 0.4 | 0.9 ± 0.4 | 0.750 |
| Absolute neutrophil (×10⁹/L) | 2.7 ± 0.7 | 2.7 ± 0.8 | 2.8 ± 0.9 | 3.0 ± 1.1 | 0.513 |
| Absolute lymphocyte (×10⁹/L) | 2.0 ± 0.4 | 2.0 ± 0.5 | 1.9 ± 0.5 | 1.9 ± 0.5 | 0.776 |
| Absolute monocyte (×10⁹/L) | 0.4 ± 0.1 | 0.5 ± 0.5 | 0.4 ± 0.1 | 0.4 ± 0.1 | 0.650 |
| Absolute eosinophil (×10⁹/L) | 0.2 ± 0.1 | 0.2 ± 0.1 | 0.1 ± 0.1 | 0.1 ± 0.1 | 0.325 |
| Absolute basophil (×10⁹/L) | 0.0 ± 0.0 | 0.0 ± 0.1 | 0.0 ± 0.1 | 0.0 ± 0.1 | 0.403 |
| Total protein (g/dL) | 7.5 ± 0.3 | 7.4 ± 0.2 | 7.4 ± 0.3 | 7.5 ± 0.4 | 0.729 |
| Albumin (g/L) | 4.5 ± 0.2 | 4.4 ± 0.2 | 4.4 ± 0.2 | 4.4 ± 0.2 | 0.016 |
| Calcium (mmol/L) | 9.5 ± 0.3 | 9.4 ± 0.2 | 9.4 ± 0.4 | 9.5 ± 0.3 | 0.493 |
| Total bilirubin (μmol/L) | 0.5 ± 0.2 | 0.5 ± 0.2 | 0.6 ± 0.3 | 0.5 ± 0.2 | 0.208 |
| SGOT (AST) (U/L) | 19.2 ± 4.8 | 19.1 ± 4.0 | 18.6 ± 3.9 | 20.8 ± 6.3 | 0.404 |
| SGPT (ALT) (U/L) | 17.0 ± 8.3 | 16.8 ± 8.7 | 16.5 ± 8.4 | 18.1 ± 9.1 | 0.334 |
| Alk. phos, total (IU/L) | 61.5 ± 15.7 | 63.8 ± 17.6 | 63.1 ± 15.1 | 62.8 ± 15.1 | 0.202 |
| Urea nitrogen (mg/dL) | 13.6 ± 3.7 | 12.4 ± 2.7 | 13.1 ± 2.5 | 12.9 ± 3.1 | 0.236 |
| Creatinine (mg/dL) | 0.9 ± 0.2 | 0.9 ± 0.2 | 0.9 ± 0.2 | 0.9 ± 0.2 | 0.782 |
| Sodium (mmol/L) | 140.2 ± 1.6 | 140.8 ± 1.9 | 141.1 ± 1.3 | 139.5 ± 2.0 | 0.054 |
| Potassium (mmol/L) | 4.2 ± 0.5 | 4.2 ± 0.4 | 4.3 ± 0.4 | 4.3 ± 0.5 | 0.438 |
| Chloride (mEq/L) | 103.4 ± 2.2 | 103.1 ± 1.9 | 103.7 ± 2.0 | 103.1 ± 2.6 | 0.683 |
| CO₂ content (mEq/L) | 24.8 ± 2.2 | 24.7 ± 1.8 | 24.1 ± 2.2 | 24.1 ± 2.3 | 0.145 |

(B)

FDR-adjusted P value (least-squares means
of linear mixed-effects model)

| | Day 11 | Day 11 | Day 11 | Day 25 | Day 25 | Day 35 |
|---|---|---|---|---|---|---|
| Parameter | vs 25 | vs 35 | vs 49 | vs 35 | vs 49 | vs 49 |
| BMI (kg/m²) | 0.930 | 0.577 | 0.489 | 0.577 | 0.489 | 0.577 |
| Fasting insulin (pmol/L) | 0.102 | 0.102 | 0.808 | 0.808 | 0.127 | 0.102 |
| Fasting glucose (mg/dL) | 0.131 | 0.003 | 0.008 | 0.095 | 0.183 | 0.562 |
| HOMA-IR | 0.078 | 0.045 | 0.531 | 0.531 | 0.210 | 0.078 |
| Triglyceride (mg/dL) | 0.732 | 0.430 | 0.732 | 0.432 | 0.937 | 0.432 |
| Total cholesterol (mg/dL) | 0.938 | 0.938 | 0.938 | 0.938 | 0.938 | 0.938 |
| HDL cholesterol (mg/dL) | 0.954 | 0.954 | 0.954 | 0.954 | 0.954 | 0.954 |
| LDL cholesterol (mg/dL) | 0.887 | 0.887 | 0.887 | 0.887 | 0.887 | 0.956 |
| Hemoglobin (g/dL) | 0.810 | 0.988 | 1.000 | 0.810 | 0.810 | 0.988 |
| Hematocrit (%) | 0.817 | 0.817 | 0.817 | 0.817 | 0.817 | 0.817 |
| Red blood count (×10¹²/L) | 0.634 | 0.978 | 0.634 | 0.634 | 0.634 | 0.634 |
| Platelet count (×10⁹/L) | 0.881 | 0.869 | 0.869 | 0.869 | 0.869 | 0.869 |
| Mean platelet volume (fL) | 1.000 | 1.000 | 1.000 | 1.000 | 1.000 | 1.000 |
| White blood count (×10⁹/L) | 0.853 | 0.853 | 0.853 | 0.853 | 0.853 | 0.853 |
| Neutrophil (%) | 0.988 | 0.458 | 0.223 | 0.458 | 0.223 | 0.458 |
| Lymphocyte (%) | 0.776 | 0.552 | 0.420 | 0.552 | 0.420 | 0.552 |
| Monocyte (%) | 0.591 | 0.698 | 0.591 | 0.680 | 0.841 | 0.680 |
| Eosinophil (%) | 0.913 | 0.913 | 0.913 | 0.913 | 0.978 | 0.913 |
| Basophil (%) | 0.883 | 0.883 | 0.883 | 0.883 | 0.883 | 0.883 |
| Absolute neutrophil (×10⁹/L) | 0.754 | 0.754 | 0.732 | 0.732 | 0.732 | 0.732 |
| Absolute lymphocyte (×10⁹/L) | 0.888 | 0.888 | 0.888 | 0.888 | 0.888 | 0.888 |
| Absolute monocyte (×10⁹/L) | 0.772 | 0.885 | 0.885 | 0.772 | 0.772 | 0.885 |
| Absolute eosinophil (×10⁹/L) | 0.553 | 0.553 | 0.478 | 0.478 | 0.478 | 0.553 |
| Absolute basophil (×10⁹/L) | 0.560 | 0.560 | 0.587 | 0.587 | 0.560 | 0.587 |
| Total protein (g/dL) | 0.918 | 0.918 | 0.918 | 0.926 | 0.926 | 0.926 |
| Albumin (g/L) | 0.023 | 0.023 | 0.023 | 1.000 | 0.890 | 0.890 |
| Calcium (mmol/L) | 0.706 | 0.706 | 0.706 | 0.706 | 0.706 | 0.745 |
| Total bilirubin (μmol/L) | 0.585 | 0.233 | 0.585 | 0.352 | 0.938 | 0.352 |
| SGOT (AST) (U/L) | 0.957 | 0.847 | 0.478 | 0.847 | 0.478 | 0.478 |
| SGPT (ALT) (U/L) | 0.821 | 0.821 | 0.465 | 0.821 | 0.465 | 0.465 |
| Alk. phos, total (IU/L) | 0.226 | 0.442 | 0.467 | 0.606 | 0.529 | 0.789 |
| Urea nitrogen (mg/dL) | 0.259 | 0.430 | 0.430 | 0.430 | 0.430 | 0.818 |
| Creatinine (mg/dL) | 0.854 | 0.854 | 0.854 | 0.854 | 0.854 | 0.854 |
| Sodium (mmol/L) | 0.406 | 0.308 | 0.349 | 0.630 | 0.106 | 0.067 |
| Potassium (mmol/L) | 0.683 | 0.619 | 0.683 | 0.619 | 0.619 | 0.683 |
| Chloride (mEq/L) | 0.778 | 0.778 | 0.778 | 0.778 | 1.000 | 0.778 |
| CO₂ content (mEq/L) | 1.000 | 0.178 | 0.178 | 0.178 | 0.178 | 1.000 |

### Example 16. Effects of Fiber Supplementation on Plasma Proteome

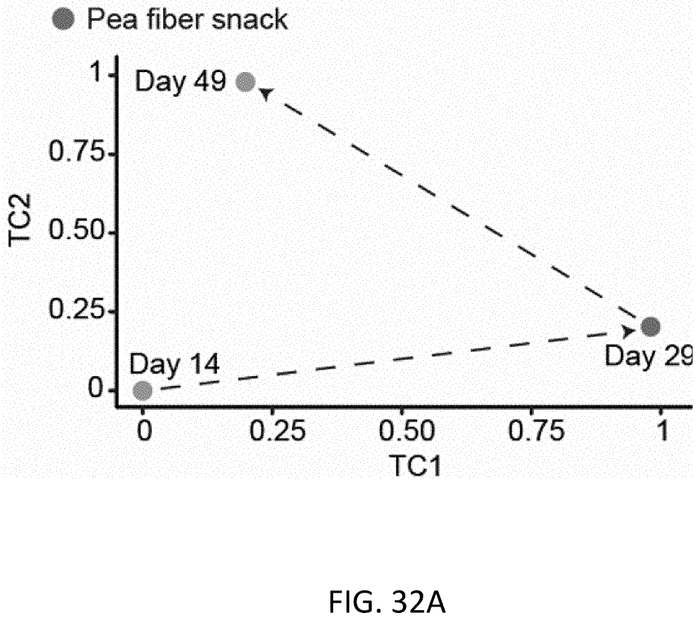

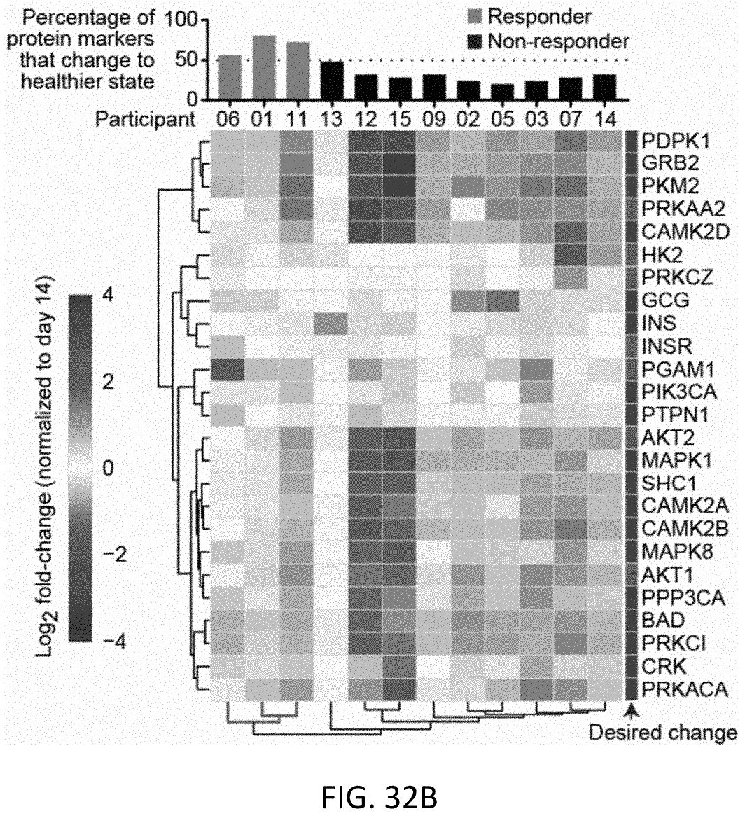

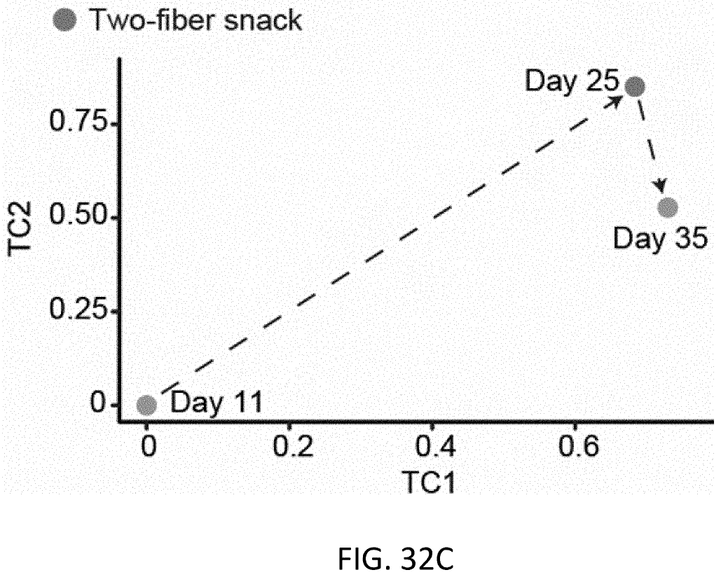

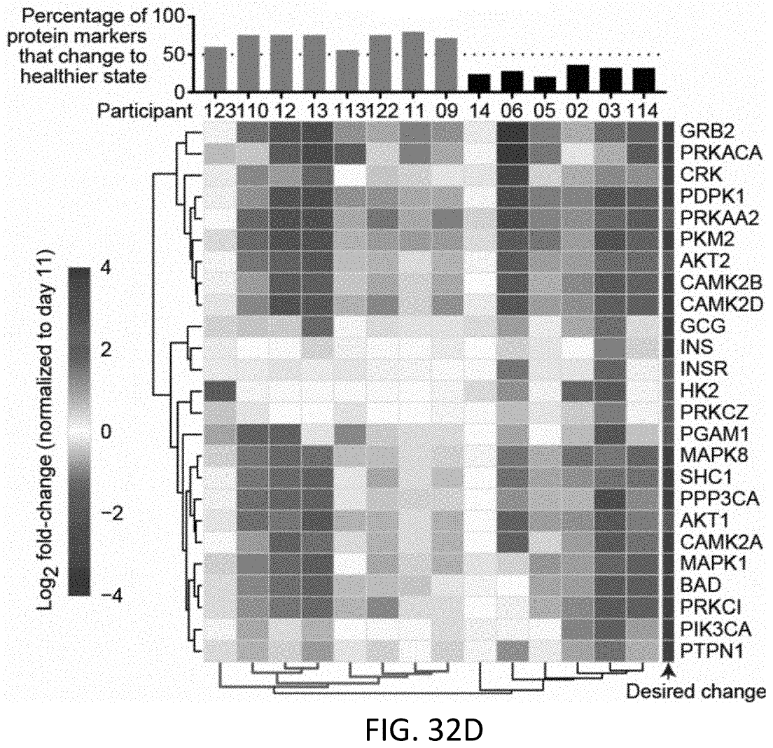

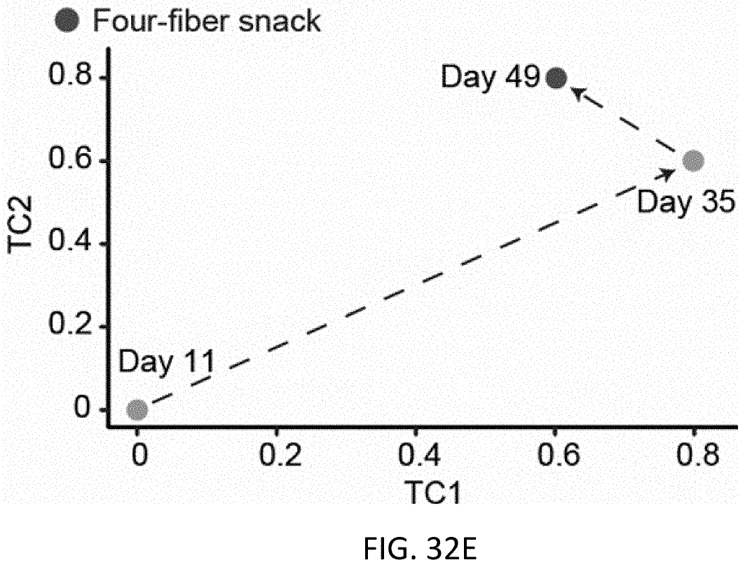

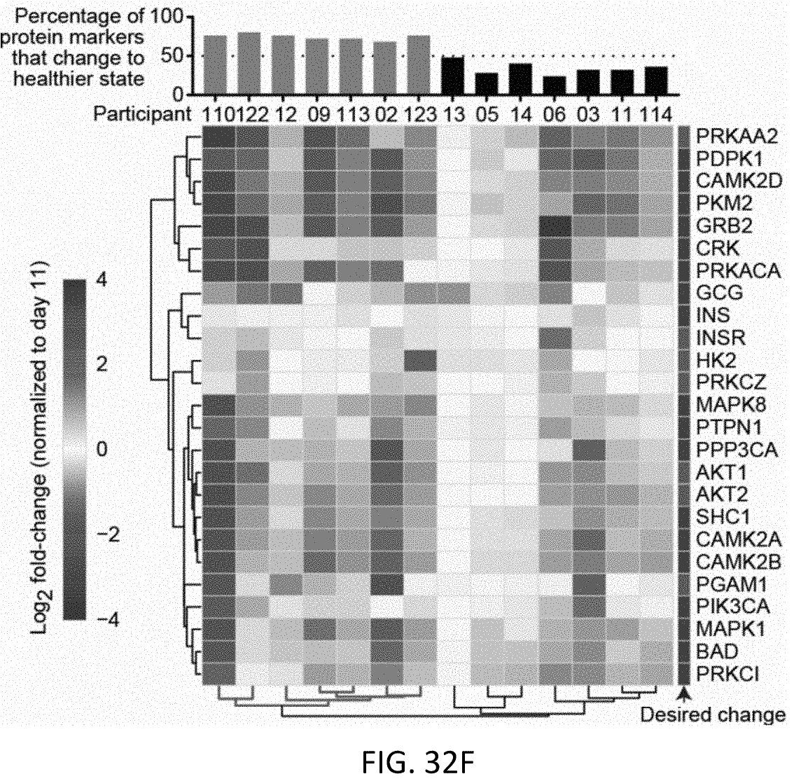

An aptamer-based platform was used to perform a quantitative multiplex proteomic analysis of the abundances of 1305 plasma proteins in subjects enrolled in both studies described in the Examples. These proteins include biomarkers and regulators of a range of physiologic, metabolic, and immunologic functions and thus provide a broad view of the effects of consuming the different snack food prototypes. A tensor comprised of subjects (rows), protein abundances (columns), and timepoints (third dimension) was created for each type of fiber intervention. For human study 1, the first tensor was made using the log₂ fold-change of plasma protein markers on day 14 (pre-intervention), day 29 (pea fiber snack at highest dose) and day 49 (post-intervention), normalized to day 14. For human study 2, second and third tensors were made using the log₂ fold-change of plasma protein markers on (i) days 11 (pre-intervention), 25 (the two-fiber snack at the highest dose) and 35 (washout phase) normalized to day 11, and (ii) days 11 (pre-intervention), 35 (washout phase) and 49 (four-fiber snack at the highest dose) normalized to day 11. [Note that because human study 2 tested the effects of two weeks of treatment with each of the snack prototypes, we used days 14, 29, and 49 (not day 35) to analyze the responses to the pea fiber snack]. The results of HO-SVD analysis of the resulting plasma proteomic datasets are described in FIG. 32.

For the first human study, TC1 distinguishes consumption of the HiSF-LoFV base diet and consumption of the maximum dose of the pea fiber snack; it also shows a return to baseline 14 days after stopping the intervention (FIG. 32A). For the second study, TC2 distinguishes the plasma proteome sampled during the pre-intervention phase on day 11 from the proteome sampled at the end of the period of consumption of the maximum dose of the two-fiber snack (FIG. 32C) and the end of the period of maximum consumption of four-fiber snack (FIG. 32E). Unlike the snack food prototype containing pea fiber alone, the plasma proteomes of participants enrolled in the multi-fiber snack study did not completely return to baseline during the washout phase (FIG. 32,E) suggesting longer-lived effects of the fiber blends.

Proteins within the 20^{th} percentile of most positive and negative projections along TC1 for human study 1 and TC2 for human study 2 were defined as most discriminatory for host responses to the different snack prototypes. Using the total number of measured plasma proteins that passed quality control (see Methods), we identified KEGG pathways significantly enriched within the set of proteins that discriminate the responses to the different fiber snack prototypes; 24 of these pathways were enriched in all three dietary interventions, including insulin signaling pathway, glucagon signaling pathway, glycolysis/gluconeogenesis, carbon metabolism, carbohydrate digestion/absorption, platelet activation, and B- and T-cell receptor signaling.

During the 2-3 week-long period of consumption of the three different snack prototypes, the four-fiber blend produced the greatest reduction in HOMA-IR (Table 22 and Table 23) although the decrease after the short 14-day period of supplementation did not achieve statistical significance (p=0.078). The log₂ fold-changes in the abundances of 25 plasma proteins in the insulin and glucagon signaling pathways for each subject are shown for all diet interventions in FIG. 32B,D,F (changes normalized to day 14 and day 11 for the first and second human studies, respectively). Hierarchical clustering (Canberra distances) of the subjects' responses revealed two distinct groups for each treatment. The direction of change in the abundance of each of these proteins that is indicative of movement towards a healthier state (based on a literature evidence; Table 24) was denoted by the bar on right side of the heatmaps (increase in red; decrease in blue). Responders were defined as those subjects with an aggregate change of ≥50% of the protein markers towards a healthier state. Combining this definition of response to a healthier state in plasma proteins markers belonging to insulin and glucagon signaling pathways with the hierarchical clustering results, we classified three of the 12 subjects in Study 1 as being responsive to pea fiber snacks (FIG. 32B), 8/14 and 7/14 subjects to the two- and four-fiber snack formulations (FIG. 32D,E).

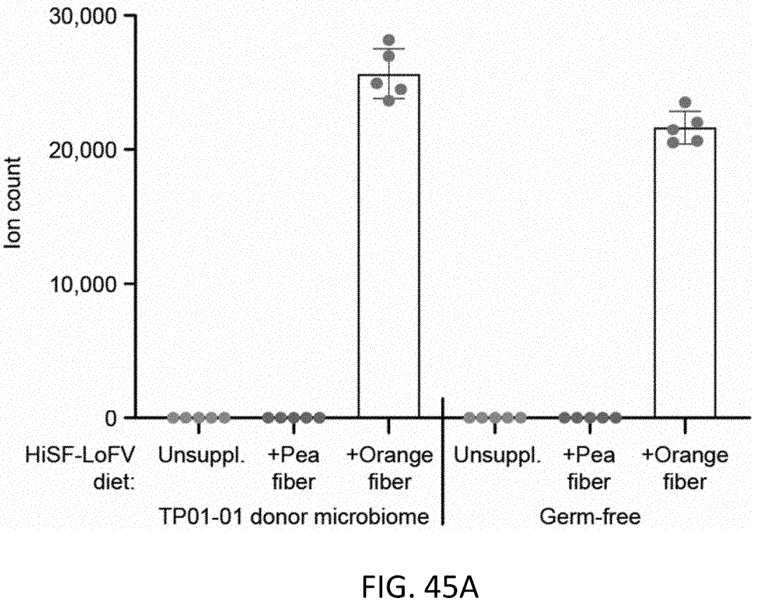

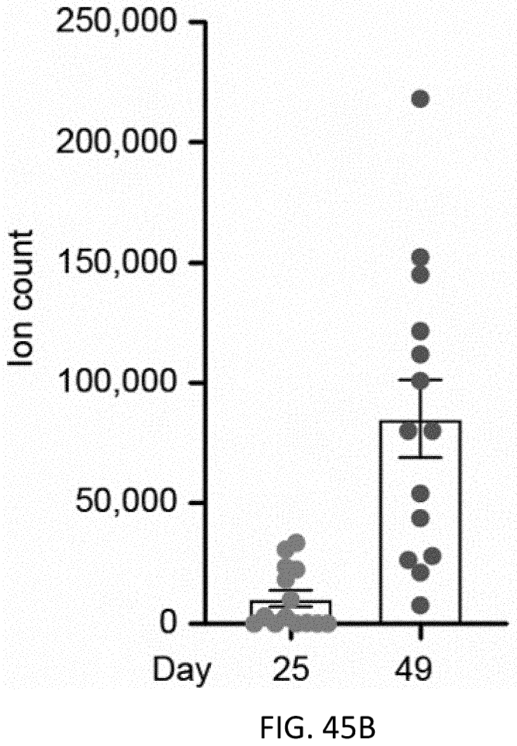

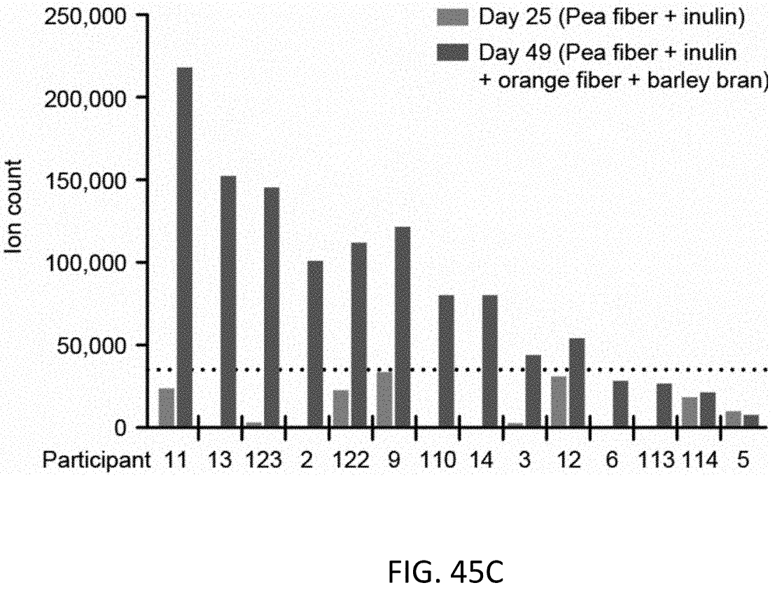

Non-targeted liquid chromatography quadrupole time-of-flight mass spectrometry (LC-QTOF-MS) of fecal samples collected from mice colonized with obese donor TP01-01 microbiota in the study shown in FIG. 30A, and fed the orange fiber-supplemented HiSF-LoFV diet, revealed an analyte with m/z of 274.1442. The m/z of 274.1442 analyte was also detected in their orange-fiber treated germ-free counterparts, but not in TP-01-01 colonized mice or germ-free controls fed the unsupplemented or pea fiber supplemented HiSF-LoFV diets (FIG. 45A). We reasoned that this m/z of 274.1442 analyte would be a useful biomarker of consumption of the four-fiber snack prototype. LC-QTOF-MS of fecal samples collected on days 25 and 49 from participants in study 2 during the time of consumption of the maximum dose of the two- and four-fiber snacks respectively, revealed significantly higher levels of the m/z of 274.1442 analyte in those consuming the orange-fiber containing snack. Interestingly, four of the eight participants who were classified as hypo-responders based on the plasma proteome response in the insulin/glucagon signaling pathways had the lowest fecal levels of the m/z 274.1442 analyte (FIG. 45B,C).

TABLE 24

| | | Entrez | Desired |
|---|---|---|---|
| Somamer ID | Protein name | Gene ID | change |
| BAD.5870.23.2 | BAD (Bcl2-associated agonist of cell death) | 572 | Decrease |
| CAMK2A.3350.53.2 | CAMK2A (Calcium/calmodulin-dependent | 815 | Decrease |
| | protein kinase type II | | |
| | subunit alpha) | | |
| CAMK2B.3351.1.1 | CAMK2B (Calcium/calmodulin-dependent | 816 | Decrease |
| | protein kinase type II | | |
| | subunit beta) | | |
| CAMK2D.3419.49.2 | CAMK2D (Calcium/calmodulin-dependent | 817 | Decrease |
| | protein kinase type II | | |
| | subunit delta) | | |
| CRK.4976.57.1 | CRK (Adapter molecule crk) | 1398 | Decrease |
| GCG.4891.50.1 | GCG (Glucagon) | 2641 | Decrease |
| GRB2.5464.52.3 | GRB2 (Growth factor receptor-bound protein 2) | 2885 | Decrease |
| INS.4883.56.2 | INS (Insulin) | 3630 | Decrease |
| MAPK1.3115.64.2 | MAPK1 (Mitogen-activated protein kinase 1) | 5594 | Decrease |
| MAPK8.3825.18.2 | MAPK8 (Mitogen-activated protein kinase 8) | 5599 | Decrease |
| PDPK1.4460.8.2 | PDPK1 (3-phosphoinositide-dependent | 5170 | Decrease |
| | protein kinase 1) | | |
| PIK3CA.PIK3R1.3390.72.2 | PIK3CA, PIK3R1 (PIK3CA/PIK3R1) | 5290, 5295 | Decrease |
| PKM2.4240.31.2 | PKM2 (Pyruvate kinase PKM) | 5315 | Decrease |
| PPP3CA.PPP3R1.4903.72.1 | PPP3CA, PPP3R1 (Calcineurin) | 5530, 5534 | Decrease |
| PRKACA.3466.8.2 | PRKACA (cAMP-dependent protein kinase | 5566 | Decrease |
| | catalytic subunit | | |
| | alpha) | | |
| PRKCI.3379.29.1 | PRKCI (Protein kinase C iota type) | 5584 | Decrease |
| PTPN1.3005.5.2 | PTPN1 (Tyrosine-protein phosphatase | 5770 | Decrease |
| | non-receptor type 1) | | |
| SHC1.5272.55.2 | SHC1 (SHC-transforming protein 1) | 6464 | Decrease |
| AKT1 AKT2.AKT3.3392.68.2 | AKT1, AKT2, AKT3 (RAC-alpha/beta/gamma | 207, 208, | Increase |
| | serine/threonine- | 10000 | |
| | protein kinase) | | |
| AKT2.5360.9.2 | AKT2 (RAC-beta serine/ | 208 | Increase |
| | threonine-protein kinase) | | |
| HK2.13130.150.3 | HK2 (Hexokinase-2) | 3099 | Increase |
| INSR.3448.13.2 | INSR (Insulin receptor) | 3643 | Increase |
| PGAM1.3896.5.2 | PGAM1 (Phosphoglycerate mutase 1) | 5223 | Increase |
| PRKAA2.PRKAB2. | PRKAA2, PRKAB2, PRKAG1 (AMP Kinase | 5563, 5565, | Increase |
| PRKAG1.5245.40.5 | (alpha2beta2gamma1)) | 5571 | |
| PRKCZ.2645.54.1 | PRKCZ (Protein kinase C zeta type) | 5590 | Increase |

### Example 17. Relating Features of the Gut Microbiome to Features of the Plasma Proteome as a Function of Fiber-Snack Consumption

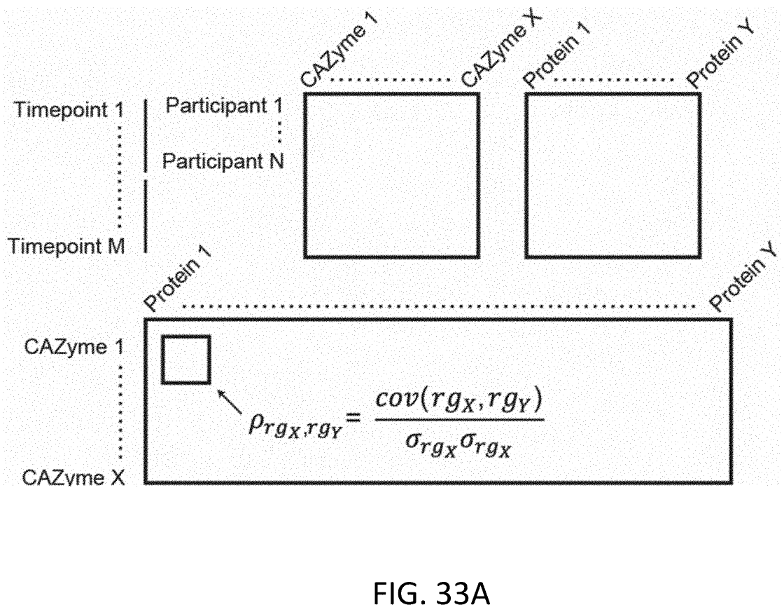

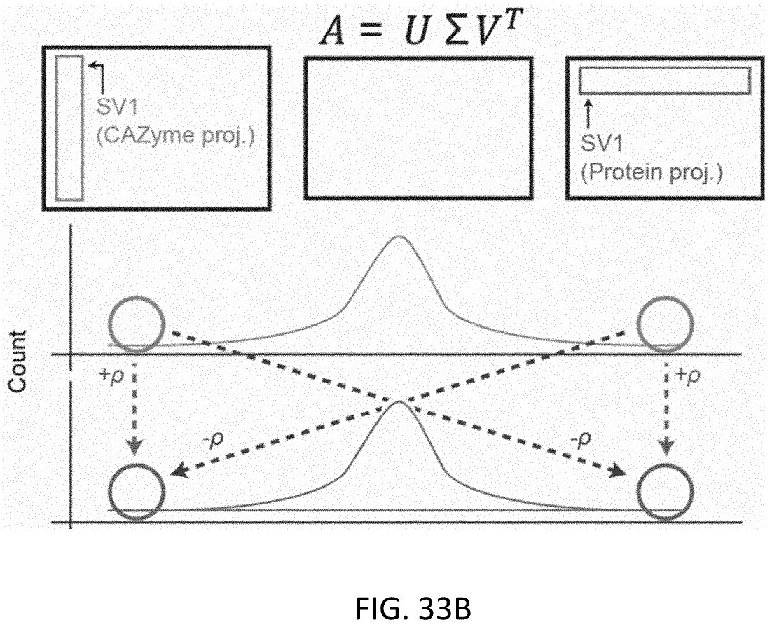

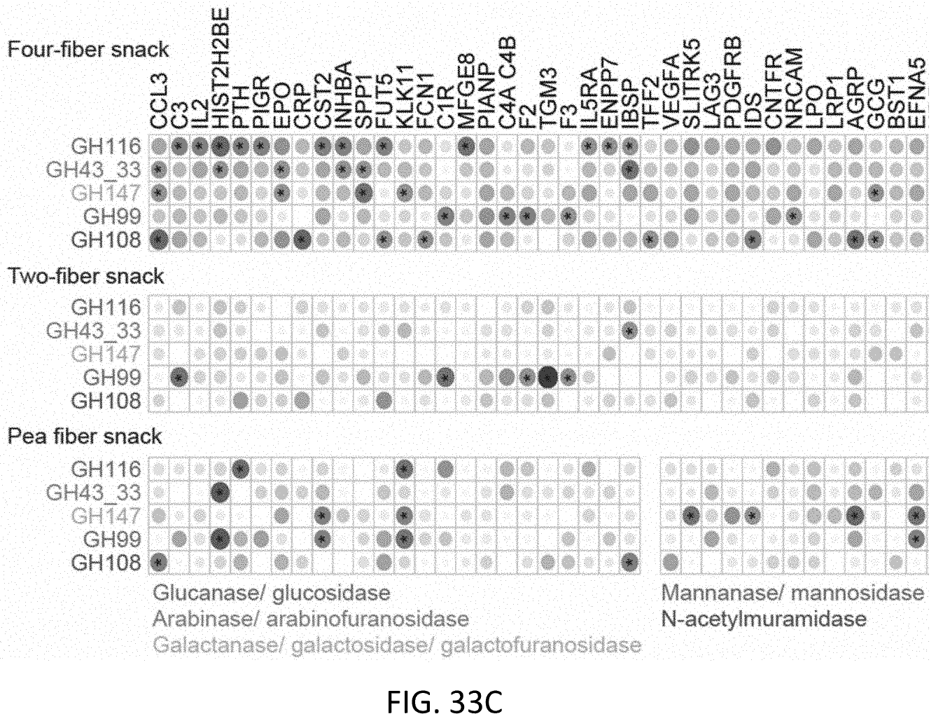

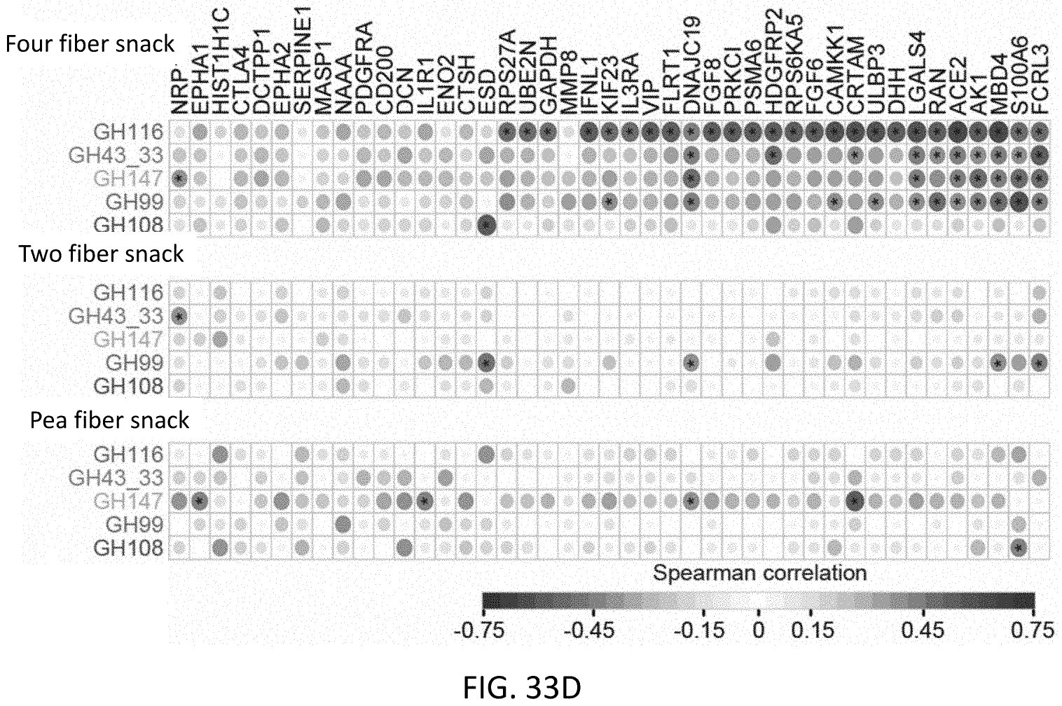

Cross-correlation singular value decomposition (CC-SVD) is a method for correlating variation in disparate feature-sets. To relate changes in the microbiome in response to different snack food prototypes and host biological status, we performed CC-SVD by creating a cross-correlation matrix where columns comprised the discriminatory plasma proteins identified by HO-SVD (i.e., the top 20^{th} percentile of most positive and negative projections along the selected TC), rows comprised the fiber-responsive CAZymes identified by HO-SVD (top 20^{th} percentile of most positive projections along the selected TC), and each element of the matrix measured the Spearman correlation between plasma protein i and CAZyme j over time. SVD was then performed on this matrix to delineate the plasma proteins and CAZymes whose variances in abundance are positively and negatively correlated. The resulting analysis provided a way to relate microbiome responses during fiber snack consumption with host responses for each subject and to discern whether there are shared features of the responses across individuals. Details of the method are provided in Methods and in FIG. 33A,B. We focused on the first singular vector (SV1) because it explained the highest percentage of the cross-correlation variance for responses to each of the snacks.

As noted above, consumption of the four-fiber snack prototype increased the abundances of genes encoding CAZymes with α-L-arabinofuranosidase (GH43_33), beta-galactosidase (GH147), endo-1,2-α-mannanase (GH99) and beta-glucosidase (GH116) activities; increases in the latter two GHs are a discriminatory feature of this multi-fiber formulation whereas both the two- and four-fiber snack prototypes increase the former two groups of CAZyme genes. CC-SVD revealed that in participants who received the four-fiber blend, these four GH families are negatively correlated with plasma proteins whose reduced abundances signal improvement to a healthier state. These included proteins involved in acute and chronic inflammation [chemokine ligand 3 (CCL3) and C-reactive protein (CRP) which are known markers of cardiovascular disease risk (21,22), secreted phosphoprotein 1 (SPP1), thrombin (F2), tissue factor (F3), vascular endothelial growth factor-A (VEGFA), platelet-derived growth factor receptor beta (PDGFRB), ephrin A5 (EFNA5), ephrin type-A receptor 1 precursor (EPHA1), ephrin type A receptor 2 precursor (EPHA2), and interleukin 1 receptor type 1 (IL1R1)]. They also included proteins involved in platelet activation and blood coagulation [complement component 3 (C3), complement receptor type 1 (C1R), complement component 4 (C4A/C4B), plasminogen activator inhibitor 1 (SERPINE1), mannan-binding lectin serine protease 1 (MASP1), and platelet-derived growth factor receptor A (PDGFRA)] (Table 25). The four GH family members showed a more coordinated negative association profile with these inflammatory proteins after supplementation with the four-fiber blend compared to supplementation with the other two snack prototypes (FIG. 33C-D).

TABLE 25
Annotated KEGG orthologies of top correlated discriminatory plasma proteins with top discriminatory CAZymes.

| | | | |
|---|---|---|---|
| | Entrez | KEGG-associated | KEGG Orthology |
| Protein name | Gene ID | function | (KO) |

(A) Study 1: pea fiber snack food prototype.

| | | | |
|---|---|---|---|
| SLPI (Antileukoproteinase) | 6590 | Proteolysis | 01002 Peptidases and inhibitors |
| IL2 (Interleukin-2) | 3558 | Lectins, Intestinal immune | 04625 C-type lectin receptor signaling |
| | | network, Adaptive immune | pathway |
| | | response | 04660 T cell receptor signaling pathway |
| | | | 04658 Th1 and Th2 cell differentiation |
| | | | 04659 Th17 cell differentiation |
| | | | 04672 Intestinal immune network for |
| | | | IgA production |
| MFGE8 (Lactadherin) | 4240 | Signal transduction | 04147 Exosome |
| TYMS (Thymidylate synthase) | 7298 | Nucleotide metabolism, | 00240 Pyrimidine metabolism |
| | | Metabolism of cofactors and | 00670 One carbon pool by folate |
| | | vitamins | |
| KPNA2 (Importin subunit | 3838 | Genetic information | 03036 Chromosome and associated |
| alpha-1) | | processing | proteins |
| S100A7 (Protein S100-A7) | 6278 | Acute and chronic | 04657 IL-17 signaling pathway |
| | | inflammation | |
| TNFRSF12A (Tumor necrosis | 51330 | Acute and chronic | 04050 Cytokine receptors |
| factor receptor superfamily | | inflammation | |
| member 12A) | | | |
| SPP1 (Osteopontin) | 6696 | Signal transduction, Acute | 04371 Apelin signaling pathway |
| | | and chronic inflammation, | 04151 PI3K-Akt signaling pathway |
| | | Innate immune response | 04512 ECM-receptor interaction |
| | | | 04510 Focal adhesion |
| | | | 04620 Toll-like receptor signaling |
| | | | pathway |
| | | | 04929 GnRH secretion |
| JAG1 (Protein jagged-1) | 182 | Signal transduction, Acute | 04330 Notch signaling pathway |
| | | and chronic inflammation, | 04371 Apelin signaling pathway |
| | | Adaptive immune response | 04668 TNF signaling pathway |
| | | | 04658 Th1 and Th2 cell differentiation |
| ENG (Endoglin) | 2022 | Signal transduction | 09183 Signaling and cellular processes |
| F2 (Thrombin) | 2147 | Signal transduction, Platelet | 04072 Phospholipase D signaling |
| | | activation and blood | pathway |
| | | coagulation, Acute and | 04080 Neuroactive ligand-receptor |
| | | chronic inflammation | interaction |
| | | | 04810 Regulation of actin cytoskeleton |
| | | | 04610 Complement and coagulation |
| | | | cascades |
| | | | 04611 Platelet activation |
| CST2 (Cystatin-SA) | 1470 | Salivary secretion | 04970 Salivary secretion |
| CCL23 (Ck-beta-8-1) | 6368 | Signal transduction, Acute | 04060 Cytokine-cytokine receptor |
| | | and chronic inflammation | interaction |
| | | | 04061 Viral protein interaction with |
| | | | cytokine and cytokine receptor |
| | | | 04062 Chemokine signaling pathway |
| | | | 04052 Cytokines and growth factors |
| APOM (Apolipoprotein M) | 55937 | — | — |
| PLA2G1B (Phospholipase A2) | 5319 | Carbon and glycan | 00564 Glycerophospholipid metabolism |
| | | biosynthesis metabolism, | 00565 Ether lipid metabolism |
| | | Signal transduction, Blood | 00590 Arachidonic acid metabolism |
| | | circulation, Fat digestion | 00591 Linoleic acid metabolism |
| | | and absorption | 00592 alpha-Linolenic acid metabolism |
| | | | 04014 Ras signaling pathway |
| | | | 04270 Vascular smooth muscle |
| | | | contraction |
| | | | 04972 Pancreatic secretion |
| | | | 04975 Fat digestion and absorption |
| MB (Myoglobin) | 4151 | Signal transduction | 02000 Transporters |
| CXCL13 (C-X-C motif | 10563 | Acute and chronic | 04060 Cytokine-cytokine receptor |
| chemokine 13) | | inflammation | interaction |
| | | | 04061 Viral protein interaction with |
| | | | cytokine and cytokine receptor |
| | | | 04062 Chemokine signaling pathway |
| | | | 04052 Cytokines and growth factors |
| IGFBP1 (Insulin-like growth | 3484 | Genetic information | 04131 Membrane trafficking |
| factor-binding protein 1) | | processing | |
| KLK5 (Kallikrein-5) | 25818 | Proteolysis | 01002 Peptidases and inhibitors |
| PIGR (Polymeric | 5284 | Intestinal immune network | 04672 Intestinal immune network for |
| immunoglobulin receptor) | | | IgA production |
| GPD1 (Glycerol-3-phosphate | 2819 | Fat digestion and absorption | 00564 Glycerophospholipid metabolism |
| dehydrogenase [NAD(+)], | | | |
| cytoplasmic) | | | |
| FABP1 (Fatty acid-binding | 2168 | Fat digestion and | 03320 PPAR signaling pathway |
| protein, liver) | | absorption, Signal | |
| | | transduction | 04975 Fat digestion and absorption |
| EPHA1 (Ephrin type-A | 2041 | Development and | 04360 Axon guidance |
| receptor 1) | | regeneration | 01001 Protein kinases |
| CST1 (Cystatin-SN) | 1469 | Salivary secretion, | 04970 Salivary secretion |
| | | Proteolysis | 01002 Peptidases and inhibitors |
| PLA2G2A (Phospholipase A2, | 5320 | Carbon and glycan | 00564 Glycerophospholipid metabolism |
| membrane associated) | | biosynthesis metabolism, | 00565 Ether lipid metabolism |
| | | Fat digestion and | 00590 Arachidonic acid metabolism |
| | | absorption, Signal | 00591 Linoleic acid metabolism |
| | | transduction, Genetic | 00592 alpha-Linolenic acid metabolism |
| | | information processing, | 04014 Ras signaling pathway |
| | | Blood circulation | 04270 Vascular smooth muscle |
| | | | contraction |
| | | | 04972 Pancreatic secretion |
| | | | 04975 Fat digestion and absorption |
| | | | 03036 Chromosome and associated |
| | | | proteins |
| LTA LTB (Lymphotoxin | 4049, | Acute and chronic | 04064 NF-kappa B signaling pathway |
| alpha1:beta2) | 4050 | inflammation, Signal | 04668 TNF signaling pathway |
| | | transduction | 04060 Cytokine-cytokine receptor |
| | | | interaction |
| | | | 04061 Viral protein interaction with |
| | | | cytokine and cytokine receptor |
| | | | 04052 Cytokines and growth factors |
| TNFRSF18 (Tumor necrosis | 8784 | Signal transduction, Acute | 04060 Cytokine-cytokine receptor |
| factor receptor superfamily | | and chronic inflammation | interaction |
| member 18) | | | 04050 Cytokine receptors |
| | | | 04090 CD molecules |
| TGM3 (Protein-glutamine | 7053 | — | 09191 Unclassified: metabolism |
| gamma-glutamyltransferase E) | | | |

(B) Study 2: two-fiber snack food prototype.

| | | | |
|---|---|---|---|
| TGFBI (Transforming growth | 7045 | Signal transduction | 99995 Signaling proteins |
| factor-beta-induced protein ig-h3) | | | |
| PLAU (Urokinase-type | 5328 | Signal transduction, Platelet | 04064 NF-kappa B signaling pathway |
| plasminogen activator) | | activation and blood | 04610 Complement and coagulation |
| | | coagulation, Acute and | cascades |
| | | chronic inflammation, | 01002 Peptidases and inhibitors |
| | | Proteolysis | 00536 Glycosaminoglycan binding |
| | | | proteins |
| CCL23 (Ck-beta-8-1) | 6368 | Signal transduction, Acute | 04060 Cytokine-cytokine receptor |
| | | and chronic inflammation | interaction |
| | | | 04061 Viral protein interaction with |
| | | | cytokine and cytokine receptor |
| | | | 04062 Chemokine signaling pathway |
| | | | 04052 Cytokines and growth factors |
| SLAMF7 (SLAM family | 57823 | Signal transduction | 04090 CD molecules |
| member 7) | | | |
| ERAP1 (Endoplasmic reticulum | 51752 | Proteolysis | 01002 Peptidases and inhibitors |
| aminopeptidase 1) | | | |
| FGF19 (Fibroblast growth | 9965 | Signal transduction | 04014 Ras signaling pathway |
| factor 19) | | | 04015 Rap1 signaling pathway |
| | | | 04010 MAPK signaling pathway |
| | | | 04151 PI3K-Akt signaling pathway |
| | | | 04810 Regulation of actin cytoskeleton |
| | | | 04052 Cytokines and growth factors |
| | | | 00536 Glycosaminoglycan binding |
| | | | proteins |
| PTH (Parathyroid hormone) | 5741 | Signal transduction | 04080 Neuroactive ligand-receptor |
| | | | interaction |
| | | | 04928 Parathyroid hormone synthesis, |
| | | | secretion and action |
| | | | 04961 Endocrine and other factor- |
| | | | regulated calcium reabsorption |
| THBS2 (Thrombospondin-2) | 7058 | Signal transduction | 04151 PI3K-Akt signaling pathway |
| | | | 04512 ECM-receptor interaction |
| | | | 04145 Phagosome |
| | | | 04510 Focal adhesion |
| | | | 04131 Membrane trafficking |
| | | | 00536 Glycosaminoglycan binding |
| | | | proteins |
| S100A7 (Protein S100-A7) | 6278 | Acute and chronic | 04657 IL-17 signaling pathway |
| | | inflammation | |
| SPINT1 (Kunitz-type protease | 6692 | Proteolysis | 01002 Peptidases and inhibitors |
| inhibitor 1) | | | |
| EPO (Erythropoietin) | 2056 | Signal transduction, Anti- | 04630 Jak-STAT signaling pathway |
| | | inflammatory | 04066 HIF-1 signaling pathway |
| | | | 04151 PI3K-Akt signaling pathway |
| | | | 04060 Cytokine-cytokine receptor |
| | | | interaction |
| | | | 04640 Hematopoietic cell lineage |
| | | | 04052 Cytokines and growth factors |
| IL18BP (Interleukin-18-binding | 10068 | Anti-inflammatory | — |
| protein) | | | |
| Human-virus (gp41 C34 peptide, | — | — | — |
| HIV) | | | |
| PCSK7 (Proprotein convertase | 9159 | Proteolysis | 01002 Peptidases and inhibitors |
| subtilisin/kexin type 7) | | | |
| | | | 03110 Chaperones and folding |
| | | | catalysts |
| ISLR2 (Immunoglobulin | 57611 | — | — |
| superfamily containing leucine- | | | |
| rich repeat protein 2) | | | |
| BST1 (ADP-ribosyl | 683 | Metabolism of cofactors and | 00760 Nicotinate and nicotinamide |
| cyclase/cyclic ADP-ribose | | vitamins, Salivary secretion, | metabolism |
| hydrolase 2) | | Signal transduction | 04970 Salivary secretion |
| | | | 04972 Pancreatic secretion |
| | | | 04090 CD molecules |
| | | | 00537 Glycosylphosphatidylinositol |
| EPHB2 (Ephrin type-B | 2048 | Development and | 04360 Axon guidance |
| receptor 2) | | regeneration | 01001 Protein kinases |
| LRP8 (Low-density lipoprotein | 7804 | Genetic information | 04131 Membrane trafficking |
| receptor-related protein 8) | | processing | |
| KIT (Mast/stem cell growth | 3815 | Signal transduction | 04014 Ras signaling pathway |
| factor receptor Kit) | | | 04015 Rap1 signaling pathway |
| | | | 04010 MAPK signaling pathway |
| | | | 04072 Phospholipase D signaling |
| | | | pathway |
| | | | 04151 PI3K-Akt signaling pathway |
| | | | 04640 Hematopoietic cell lineage |
| | | | 04916 Melanogenesis |
| HSPA1A (Heat shock 70 kDa | 3303 | Genetic information | 03040 Spliceosome |
| protein 1A) | | processing, Signal | 04141 Protein processing in |
| | | transduction, Proteolysis | endoplasmic reticulum |
| | | | 04010 MAPK signaling pathway |
| | | | 04144 Endocytosis |
| | | | 04612 Antigen processing and |
| | | | presentation |
| | | | 04915 Estrogen signaling pathway |
| | | | 04213 Longevity regulating pathway - |
| | | | multiple species |
| | | | 01009 Protein phosphatases and |
| | | | associated proteins |
| | | | 03009 Ribosome biogenesis |
| | | | 03110 Chaperones and folding |
| | | | catalysts |
| | | | 04131 Membrane trafficking |
| | | | 03051 Proteasome |
| | | | 03029 Mitochondrial biogenesis |
| | | | 04147 Exosome |
| KLK8 (Kallikrein-8) | 11202 | Proteolysis | 01002 Peptidases and inhibitors |
| SFRP1 (Secreted frizzled- | 6422 | Signal transduction, | 04310 Wnt signaling pathway |
| related protein 1) | | Development and | |
| | | regeneration | |
| TPSG1 (Tryptase gamma) | 25823 | Proteolysis | 01002 Peptidases and inhibitors |
| NRCAM (Neuronal cell adhesion | 4897 | Signal transduction | 04514 Cell adhesion molecules |
| molecule) | | | (CAMs) |
| IL18R1 (Interleukin-18 | 8809 | Signal transduction, Acute | 04668 TNF signaling pathway |
| receptor 1) | | and chronic inflammation | 04060 Cytokine-cytokine receptor |
| | | | interaction |
| | | | 04061 Viral protein interaction with |
| | | | cytokine and cytokine receptor |
| | | | 04050 Cytokine receptors |
| | | | 04090 CD molecules |
| SHH (Sonic hedgehog protein) | 6469 | Signal transduction, | 04340 Hedgehog signaling pathway |
| | | Development and | 04360 Axon guidance |
| | | regeneration, Proteolysis | 01002 Peptidases and inhibitors |
| | | | 00536 Glycosaminoglycan binding |
| | | | proteins |
| ANGPT2 (Angiopoietin-2) | 285 | Signal transduction | 04014 Ras signaling pathway |
| | | | 04015 Rap1 signaling pathway |
| | | | 04010 MAPK signaling pathway |
| | | | 04066 HIF-1 signaling pathway |
| | | | 04151 PI3K-Akt signaling pathway |
| FLRT2 (Leucine-rich repeat | 23768 | Signal transduction | 99995 Signaling proteins |
| transmembrane protein FLRT2) | | | |
| CD36 (Platelet glycoprotein 4) | 948 | Signal transduction, | 04152 AMPK signaling pathway |
| | | Development and | 04512 ECM-receptor interaction |
| | | regeneration, Fat digestion | 04145 Phagosome |
| | | and absorption | 04640 Hematopoietic cell lineage |
| | | | 04920 Adipocytokine signaling |
| | | | pathway |
| | | | 03320 PPAR signaling pathway |
| | | | 04975 Fat digestion and absorption |
| | | | 04979 Cholesterol metabolism |
| | | | 04131 Membrane trafficking |
| | | | 04147 Exosome |
| | | | 04090 CD molecules |
| MMP12 (Macrophage | 4321 | Proteolysis | 01002 Peptidases and inhibitors |
| metalloelastase) | | | |
| EDA (Ectodysplasin-A, | 1896 | Signal transduction | 04064 NF-kappa B signaling pathway |
| secreted form) | | | 04060 Cytokine-cytokine receptor |
| | | | interaction |
| | | | 04052 Cytokines and growth factors |
| PYY (Peptide YY) | 5697 | Feeding behavior, Signal | 04080 Neuroactive ligand-receptor |
| | | transduction | interaction |
| LRP1 (Low-density lipoprotein | 4035 | Fat digestion and absorption | 04979 Cholesterol metabolism |
| receptor-related protein 1, | | | 04131 Membrane trafficking |
| soluble) | | | 04090 CD molecules |
| LRRTM1 (Leucine-rich repeat | 347730 | Signal transduction | 99995 Signaling proteins |
| transmembrane neuronal | | | |
| protein 1) | | | |
| LIFR (Leukemia inhibitory factor | 3977 | Signal transduction, Anti- | 04630 Jak-STAT signaling pathway |
| receptor) | | inflammatory | 04060 Cytokine-cytokine receptor |
| | | | interaction |
| | | | 04550 Signaling pathways regulating |
| | | | pluripotency of stem cells |
| | | | 04050 Cytokine receptors |
| | | | 04090 CD molecules |
| PDGFRA (Platelet-derived | 5156 | Signal transduction, Platelet | 04014 Ras signaling pathway |
| growth factor receptor alpha) | | activation and blood | 04015 Rap1 signaling pathway |
| | | coagulation | 04010 MAPK signaling pathway |
| | | | 04630 Jak-STAT signaling pathway |
| | | | 04020 Calcium signaling pathway |
| | | | 04072 Phospholipase D signaling |
| | | | pathway |
| | | | 04151 PI3K-Akt signaling pathway |
| | | | 04144 Endocytosis |
| | | | 04510 Focal adhesion |
| | | | 04540 Gap junction |
| | | | 04810 Regulation of actin cytoskeleton |
| | | | 01001 Protein kinases |
| | | | 04090 CD molecules |
| TNFSF13B (Tumor necrosis | 10673 | Signal transduction, Intestinal | 04064 NF-kappa B signaling pathway |
| factor ligand superfamily | | immune network | 04060 Cytokine-cytokine receptor |
| member 13B) | | | interaction |
| | | | 04672 Intestinal immune network for |
| | | | IgA production |
| | | | 04052 Cytokines and growth factors |
| | | | 04090 CD molecules |
| INSR (Insulin receptor) | 3643 | Signal transduction, Insulin | 04014 Ras signaling pathway |
| | | signaling pathway, Fat | 04015 Rap1 signaling pathway |
| | | digestion and absorption | 04010 MAPK signaling pathway |
| | | | 04066 HIF-1 signaling pathway |
| | | | 04068 FoxO signaling pathway |
| | | | 04072 Phospholipase D signaling |
| | | | pathway |
| | | | 04022 cGMP-PKG signaling pathway |
| | | | 04151 PI3K-Akt signaling pathway |
| | | | 04152 AMPK signaling pathway |
| | | | 04150 mTOR signaling pathway |
| | | | 04520 Adherens junction |
| | | | 04910 Insulin signaling pathway |
| | | | 04923 Regulation of lipolysis in |
| | | | adipocytes |
| | | | 04913 Ovarian steroidogenesis |
| | | | 04960 Aldosterone-regulated sodium |
| | | | reabsorption |
| | | | 04211 Longevity regulating pathway |
| | | | 04213 Longevity regulating pathway - |
| | | | multiple species |
| | | | 01001 Protein kinases |
| | | | 04090 CD molecules |
| TEK (Angiopoietin-1 receptor, | 7010 | Signal transduction | 04014 Ras signaling pathway |
| soluble) | | | 04015 Rap1 signaling pathway |
| | | | 04010 MAPK signaling pathway |
| | | | 04066 HIF-1 signaling pathway |
| | | | 04151 PI3K-Akt signaling pathway |
| | | | 01001 Protein kinases |
| | | | 04090 CD molecules |
| LRIG3 (Leucine-rich repeats | 121227 | — | — |
| and immunoglobulin-like | | | |
| domains protein 3) | | | |
| WFIKKN2 (WAP, Kazal, | 124857 | Proteolysis | 01002 Peptidases and inhibitors |
| immunoglobulin, Kunitz and | | | |
| NTR domain-containing protein | | | |
| 2) | | | |
| TFF1 (Trefoil factor 1) | 7031 | Signal transduction | 04915 Estrogen signaling pathway |
| MDH1 (Malate dehydrogenase, | 4190 | Carbon and glycan | 00020 Citrate cycle (TCA cycle) |
| cytoplasmic) | | biosynthesis metabolism | 00620 Pyruvate metabolism |
| | | | 00630 Glyoxylate and dicarboxylate |
| | | | metabolism |
| | | | 00270 Cysteine and methionine |
| | | | metabolism |
| | | | 04964 Proximal tubule bicarbonate |
| | | | reclamation |
| SFTPD (Pulmonary surfactant- | 6441 | Genetic information | 04145 Phagosome |
| associated protein D) | | processing, Lectins | 04131 Membrane trafficking |
| | | | 04091 Lectins |
| BCL2 (Apoptosis regulator | 596 | Signal transduction, Genetic | 04141 Protein processing in |
| Bcl-2) | | information processing, | endoplasmic reticulum |
| | | Proteolysis, AGE-RAGE | 04340 Hedgehog signaling pathway |
| | | signaling pathway in diabetic | 04630 Jak-STAT signaling pathway |
| | | complications | 04064 NF-kappa B signaling pathway |
| | | | 04066 HIF-1 signaling pathway |
| | | | 04071 Sphingolipid signaling pathway |
| | | | 04151 PI3K-Akt signaling pathway |
| | | | 04140 Autophagy - animal |
| | | | 04210 Apoptosis |
| | | | 04217 Necroptosis |
| | | | 04115 p53 signaling pathway |
| | | | 04510 Focal adhesion |
| | | | 04621 NOD-like receptor signaling |
| | | | pathway |
| | | | 04915 Estrogen signaling pathway |
| | | | 04928 Parathyroid hormone synthesis, |
| | | | secretion and action |
| | | | 04261 Adrenergic signaling in |
| | | | cardiomyocytes |
| | | | 04725 Cholinergic synapse |
| | | | 04722 Neurotrophin signaling pathway |
| | | | 01009 Protein phosphatases and |
| | | | associated proteins |
| ICOS (Inducible T-cell | 29851 | Adaptive immune response, | 04514 Cell adhesion molecules |
| costimulator) | | Intestinal immune network | (CAMs) |
| | | | 04660 T cell receptor signaling |
| | | | pathway |
| | | | 04672 Intestinal immune network for |
| | | | IgA production |
| | | | 04090 CD molecules |
| HIST1H1C (Histone H1.2) | 3006 | Genetic information | 03036 Chromosome and associated |
| | | processing | proteins |
| SERPINE2 (Glia-derived nexin) | 5270 | Proteolysis | 01002 Peptidases and inhibitors |
| | | | 00536 Glycosaminoglycan binding |
| | | | proteins |
| IL16 (Interleukin-16) | 3603 | Acute and chronic | 04060 Cytokine-cytokine receptor |
| | | inflammation | interaction |
| | | | 04052 Cytokines and growth factors |
| KIR2DL4 (Killer cell | 3805 | Innate immune response | 04218 Cellular senescence |
| immunoglobulin-like receptor | | | 04650 Natural killer cell mediated |
| 2DL4) | | | cytotoxicity |
| | | | 04612 Antigen processing and |
| | | | presentation |
| | | | 04090 CD molecules |
| CXCL3 CXCL2 (Gro- | 2921 | Acute and chronic | 04064 NF-kappa B signaling pathway |
| beta/gamma) | | inflammation | 04668 TNF signaling pathway |
| | | | 04060 Cytokine-cytokine receptor |
| | | | interaction |
| | | | 04061 Viral protein interaction with |
| | | | cytokine and cytokine receptor |
| | | | 04621 NOD-like receptor signaling |
| | | | pathway |
| | | | 04657 IL-17 signaling pathway |
| | | | 04062 Chemokine signaling pathway |
| | | | 04052 Cytokines and growth factors |
| PRDX1 (Peroxiredoxin-1) | 5052 | Transport and catabolism, | 04146 Peroxisome |
| | | Signal transduction, | 04147 Exosome |
| CMA1 (Chymase) | 1215 | Blood circulation, Proteolysis | 04614 Renin-angiotensin system |
| | | | 01002 Peptidases and inhibitors |
| CCL13 (C-C motif | 6357 | Acute and chronic | 04064 NF-kappa B signaling pathway |
| chemokine 13) | | inflammation | 04060 Cytokine-cytokine receptor |
| | | | interaction |
| | | | 04061 Viral protein interaction with |
| | | | cytokine and cytokine receptor |
| | | | 04062 Chemokine signaling pathway |
| | | | 04052 Cytokines and growth factors |
| | | | 00536 Glycosaminoglycan binding |
| | | | proteins |
| RSPO2 (R-spondin-2) | 340419 | Signal transduction, | 04310 Wnt signaling pathway |
| | | Development and | |
| | | regeneration | |
| RPS3A (40S ribosomal protein | 6189 | Genetic information | 03010 Ribosome |
| S3a) | | processing | |
| HNRNPA2B1 (Heterogeneous | 3181 | Genetic information | 03019 Messenger RNA biogenesis |
| nuclear ribonucleoproteins | | processing, Signal | 03041 Spliceosome |
| A2/B1) | | transduction | 04147 Exosome |
| BPI (Bactericidal permeability- | 671 | — | — |
| increasing protein) | | | |
| EPB41 (Protein 4.1) | 2035 | Signal transduction | 04812 Cytoskeleton proteins |
| TBP (TATA-box-binding protein) | 6908 | Genetic information | 03022 Basal transcription factors |
| | | processing | |
| RBM39 (RNA-binding protein | 9584 | Genetic information | 03041 Spliceosome |
| 39) | | processing | |
| BSG (Basigin) | 682 | Signal transduction | 04147 Exosome |
| | | | 04090 CD molecules |
| SOD1 (Superoxide dismutase | 6647 | Transport and catabolism | 04146 Peroxisome |
| [Cu—Zn]) | | | |
| ACVR1B (Activin receptor type- | 91 | Aging | 04213 Longevity regulating pathway - |
| 1B) | | | multiple species |
| S100A9 (Protein S100-A9) | 6280 | Acute and chronic | 04657 IL-17 signaling pathway |
| | | inflammation | |
| SH2D1A (SH2 domain- | 4068 | Innate immune response | 04650 Natural killer cell mediated |
| containing protein 1A) | | | cytotoxicity |
| SMPDL3A (Acid | 10924 | — | 09191 Unclassified: metabolism |
| sphingomyelinase-like | | | |
| phosphodiesterase 3a) | | | |

(C) Study 2: four-fiber snack food prototype.

| | | | |
|---|---|---|---|
| CCL3 (C-C motif chemokine 3) | 6348 | Acute and chronic inflammation | 04060 Cytokine-cytokine receptor |
| | | | interaction |
| | | | 04061 Viral protein interaction with |
| | | | cytokine and cytokine receptor |
| | | | 04620 Toll-like receptor signaling |
| | | | pathway |
| | | | 04062 Chemokine signaling pathway |
| | | | 04052 Cytokines and growth factors |
| | | | 00536 Glycosaminoglycan binding |
| | | | proteins |
| C3 (C3a anaphylatoxin) | 718 | Innate immune response, | 04080 Neuroactive ligand-receptor |
| | | Platelet activation and blood | interaction |
| | | coagulation | 04145 Phagosome |
| | | | 04610 Complement and coagulation |
| | | | cascades |
| | | | 01002 Peptidases and inhibitors |
| | | | 04131 Membrane trafficking |
| | | | 04147 Exosome |
| IL2 (Interleukin-2) | 3558 | Signal transduction, Anti- | 04625 C-type lectin receptor |
| | | inflammatory, Intestinal immune | signaling pathway |
| | | network, Adaptive immune | 04660 T cell receptor signaling |
| | | response | pathway |
| | | | 04658 Th1 and Th2 cell |
| | | | differentiation |
| | | | 04659 Th17 cell differentiation |
| | | | 04672 Intestinal immune network for |
| | | | IgA production |
| HIST2H2BE (Histone H2B type | 8349 | Genetic information processing | 03036 Chromosome and associated |
| 2-E) | | | proteins |
| | | | 04147 Exosome |
| PTH (Parathyroid hormone) | 5741 | Signal transduction | 04080 Neuroactive ligand-receptor |
| | | | interaction |
| | | | 04928 Parathyroid hormone |
| | | | synthesis, secretion and action |
| | | | 04961 Endocrine and other factor- |
| | | | regulated calcium reabsorption |
| PIGR (Polymeric | 5284 | Intestinal immune network | 04672 Intestinal immune network for |
| immunoglobulin receptor) | | | IgA production |
| EPO (Erythropoietin) | 2056 | Signal transduction, Anti- | 04630 Jak-STAT signaling pathway |
| | | inflammatory | 04066 HIF-1 signaling pathway |
| | | | 04151 PI3K-Akt signaling pathway |
| | | | 04060 Cytokine-cytokine receptor |
| | | | interaction |
| | | | 04640 Hematopoietic cell lineage |
| | | | 04052 Cytokines and growth factors |
| CRP (C-reactive protein) | 1401 | AGE-RAGE signaling pathway | 09193 Unclassified: signaling and |
| | | in diabetic complications, Acute | cellular processes |
| | | and chronic inflammation | |
| CST2 (Cystatin-SA) | 1470 | Salivary secretion | 04970 Salivary secretion |
| INHBA (Inhibin beta A chain) | 3624 | Signal transduction | 04350 TGF-beta signaling pathway |
| | | | 04060 Cytokine-cytokine receptor |
| | | | interaction |
| | | | 04550 Signaling pathways regulating |
| | | | pluripotency of stem cells |
| | | | 04052 Cytokines and growth factors |
| SPP1 (Osteopontin) | 6696 | Signal transduction, Acute and | 04371 Apelin signaling pathway |
| | | chronic inflammation, Innate | 04151 PI3K-Akt signaling pathway |
| | | immune response | 04512 ECM-receptor interaction |
| | | | 04510 Focal adhesion |
| | | | 04620 Toll-like receptor signaling |
| | | | pathway |
| | | | 04929 GnRH secretion |
| FUT5 (Alpha-(1,3)- | 2527 | Carbon and glycan biosynthesis | 00601 Glycosphingolipid |
| fucosyltransferase 5) | | metabolism | biosynthesis - lacto and neolacto |
| | | | series |
| | | | 01003 Glycosyltransferases |
| KLK11 (Kallikrein-11) | 11012 | Proteolysis | 01002 Peptidases and inhibitors |
| FCN1 (Ficolin-1) | 2219 | Lectins | 04091 Lectins |
| C1R (Complement C1r | 715 | Platelet activation and blood | 04145 Phagosome |
| subcomponent) | | coagulation, Proteolysis | 04610 Complement and coagulation |
| | | | cascades |
| | | | 01002 Peptidases and inhibitors |
| | | | 04131 Membrane trafficking |
| MFGE8 (Lactadherin) | 4240 | Signal transduction | 04147 Exosome |
| PIANP (PILR alpha-associated | 196500 | — | — |
| neural protein) | | | |
| C4A C4B (Complement C4b) | 720, 721 | Platelet activation and blood | 04610 Complement and coagulation |
| | | coagulation, Proteolysis | cascades |
| | | | 01002 Peptidases and inhibitors |
| | | | 04147 Exosome |
| F2 (Thrombin) | 2147 | Signal transduction, Platelet | 04072 Phospholipase D signaling |
| | | activation and blood | pathway |
| | | coagulation, Acute and chronic | 04080 Neuroactive ligand-receptor |
| | | inflammation | interaction |
| | | | 04810 Regulation of actin |
| | | | cytoskeleton |
| | | | 04610 Complement and coagulation |
| | | | cascades |
| | | | 04611 Platelet activation |
| TGM3 (Protein-glutamine | 7053 | — | 09191 Unclassified: metabolism |
| gamma-glutamyltransferase E) | | | |
| F3 (Tissue Factor) | 2152 | Platelet activation and blood | 04610 Complement and coagulation |
| | | coagulation, Acute and chronic | cascades |
| | | inflammation | 04090 CD molecules |
| | | | 00537 Glycosylphosphatidylinositol |
| | | | (GPI)-anchored proteins |
| IL5RA (Interleukin-5 receptor | 3568 | Signal transduction, Innate | 04630 Jak-STAT signaling pathway |
| subunit alpha) | | immune response | 04060 Cytokine-cytokine receptor |
| | | | interaction |
| | | | 04640 Hematopoietic cell lineage |
| | | | 04050 Cytokine receptors |
| | | | 04090 CD molecules |
| ENPP7 (Ectonucleotide | 339221 | Carbon and glycan biosynthesis | 00600 Sphingolipid metabolism |
| pyrophosphatase/phosphodiest | | metabolism | |
| erase family member 7) | | | |
| IBSP (Bone sialoprotein 2) | 3381 | Signal transduction | 04151 PI3K-Akt signaling pathway |
| | | | 04512 ECM-receptor interaction |
| | | | 04510 Focal adhesion |
| TFF2 (Trefoil factor 2) | 7032 | — | 99992 Structural proteins |
| VEGFA (Vascular endothelial | 7422 | Signal transduction, Acute and | 04014 Ras signaling pathway |
| growth factor A) | | chronic inflammation | 04015 Rap1 signaling pathway |
| | | | 04010 MAPK signaling pathway |
| | | | 04370 VEGF signaling pathway |
| | | | 04066 HIF-1 signaling pathway |
| | | | 04151 PI3K-Akt signaling pathway |
| | | | 04510 Focal adhesion |
| | | | 04926 Relaxin signaling pathway |
| | | | 04052 Cytokines and growth factors |
| | | | 00536 Glycosaminoglycan binding |
| | | | proteins |
| SLITRK5 (SLIT and NTRK-like | 26050 | — | — |
| protein 5) | | | |
| LAG3 (Lymphocyte activation | 3902 | Signal transduction | 04090 CD molecules |
| gene 3 protein) | | | |
| PDGFRB (Platelet-derived | 5159 | Signal transduction, Acute and | 04014 Ras signaling pathway |
| growth factor receptor beta) | | chronic inflammation | 04015 Rap1 signaling pathway |
| | | | 04010 MAPK signaling pathway |
| | | | 04630 Jak-STAT signaling pathway |
| | | | 04020 Calcium signaling pathway |
| | | | 04072 Phospholipase D signaling |
| | | | pathway |
| | | | 04151 PI3K-Akt signaling pathway |
| | | | 04510 Focal adhesion |
| | | | 04540 Gap junction |
| | | | 04810 Regulation of actin |
| | | | cytoskeleton |
| | | | 01001 Protein kinases |
| | | | 04090 CD molecules |
| IDS (Iduronate 2-sulfatase) | 3423 | Carbon and glycan biosynthesis | 00531 Glycosaminoglycan |
| | | metabolism | degradation |
| | | | 04142 Lysosome |
| CNTFR (Ciliary neurotrophic | 1271 | Signal transduction | 04630 Jak-STAT signaling pathway |
| factor receptor subunit alpha) | | | 04060 Cytokine-cytokine receptor |
| | | | interaction |
| | | | 04050 Cytokine receptors |
| | | | 00537 Glycosylphosphatidylinositol |
| | | | (GPI)-anchored proteins |
| NRCAM (Neuronal cell | 4897 | Signal transduction | 04514 Cell adhesion molecules |
| adhesion molecule) | | | (CAMs) |
| LPO (Lactoperoxidase) | 4025 | Salivary secretion | 04970 Salivary secretion |
| LRP1 (Low-density lipoprotein | 4035 | Fat digestion and absorption | 04979 Cholesterol metabolism |
| receptor-related protein 1, | | | 04131 Membrane trafficking |
| soluble) | | | 04090 CD molecules |
| AGRP (Agouti-related protein) | 181 | Feeding behavior, Signal | 04920 Adipocytokine signaling |
| | | transduction | pathway |
| GCG (Glucagon) | 2641 | Feeding behavior, Glucagon | 04024 cAMP signaling pathway |
| | | signaling pathway | 04080 Neuroactive ligand-receptor |
| | | | interaction |
| | | | 04911 Insulin secretion |
| | | | 04922 Glucagon signaling pathway |
| | | | 04714 Thermogenesis |
| BST1 (ADP-ribosyl | 683 | Metabolism of cofactors and | 00760 Nicotinate and nicotinamide |
| cyclase/cyclic ADP-ribose | | vitamins, Salivary secretion, | metabolism |
| hydrolase 2) | | Signal transduction | 04970 Salivary secretion |
| | | | 04972 Pancreatic secretion |
| | | | 04090 CD molecules |
| | | | 00537 Glycosylphosphatidylinositol |
| EFNA5 (Ephrin-A5) | 1946 | Signal transduction, | 04014 Ras signaling pathway |
| | | Development and regeneration, | 04015 Rap1 signaling pathway |
| | | Acute and chronic inflammation | 04010 MAPK signaling pathway |
| | | | 04151 PI3K-Akt signaling pathway |
| | | | 04360 Axon guidance |
| | | | 04052 Cytokines and growth factors |
| | | | 00536 Glycosaminoglycan binding |
| | | | proteins |
| NRP1 (Neuropilin-1) | 8829 | Development and regeneration, | 04360 Axon guidance |
| | | Signal transduction | 04090 CD molecules |
| EPHA1 (Ephrin type-A | 2041 | Development and regeneration, | 04360 Axon guidance |
| receptor 1) | | Acute and chronic inflammation | 01001 Protein kinases |
| HIST1H1C (Histone H1.2) | 3006 | Genetic information processing | 03036 Chromosome and associated |
| | | | proteins |
| CTLA4 (Cytotoxic T- | 1493 | Signal transduction, Adaptive | 04514 Cell adhesion molecules |
| lymphocyte protein 4) | | immune response | (CAMs) |
| | | | 04660 T cell receptor signaling |
| | | | pathway |
| | | | 04090 CD molecules |
| DCTPP1 (dCTP | 79077 | Nucleotide metabolism | 00240 Pyrimidine metabolism |
| pyrophosphatase 1) | | | |
| EPHA2 (Ephrin type-A | 1969 | Signal transduction, | 04014 Ras signaling pathway |
| receptor 2) | | Development and regeneration, | 04015 Rap1 signaling pathway |
| | | Acute and chronic inflammation | 04010 MAPK signaling pathway |
| | | | 04151 PI3K-Akt signaling pathway |
| | | | 04360 Axon guidance |
| | | | 01001 Protein kinases |
| SERPINE1 (Plasminogen | 5054 | Signal transduction, Platelet | 04390 Hippo signaling pathway |
| activator inhibitor 1) | | activation and blood | 04371 Apelin signaling pathway |
| | | coagulation, Proteolysis | 04066 HIF-1 signaling pathway |
| | | | 04115 p53 signaling pathway |
| | | | 04218 Cellular senescence |
| | | | 04610 Complement and coagulation |
| | | | cascades |
| | | | 01002 Peptidases and inhibitors |
| | | | 04147 Exosome |
| | | | 00536 Glycosaminoglycan binding |
| | | | proteins |
| MASP1 (Mannan-binding lectin | 5648 | Platelet activation and blood | 04610 Complement and coagulation |
| serine protease 1) | | coagulation, Proteolysis | cascades |
| | | | 01002 Peptidases and inhibitors |
| NAAA (N-acylethanolamine- | 27163 | Proteolysis | 01002 Peptidases and inhibitors |
| hydrolyzing acid amidase) | | | |
| PDGFRA (Platelet-derived | 5156 | Signal transduction, Platelet | 04014 Ras signaling pathway |
| growth factor receptor alpha) | | activation and blood coagulation | 04015 Rap1 signaling pathway |
| | | | 04010 MAPK signaling pathway |
| | | | 04630 Jak-STAT signaling pathway |
| | | | 04020 Calcium signaling pathway |
| | | | 04072 Phospholipase D signaling |
| | | | pathway |
| | | | 04151 PI3K-Akt signaling pathway |
| | | | 04144 Endocytosis |
| | | | 04510 Focal adhesion |
| | | | 04540 Gap junction |
| | | | 04810 Regulation of actin |
| | | | cytoskeleton |
| | | | 01001 Protein kinases |
| | | | 04090 CD molecules |
| CD200 (OX-2 membrane | 4345 | Signal transduction | 04090 CD molecules |
| glycoprotein) | | | |
| DCN (Decorin) | 1634 | Signal transduction | 04350 TGF-beta signaling pathway |
| | | | 00535 Proteoglycans |
| IL1R1 (Interleukin-1 receptor | 3554 | Signal transduction, Platelet | 04010 MAPK signaling pathway |
| type 1) | | activation and blood | 04064 NF-kappa B signaling pathway |
| | | coagulation, Acute and chronic | 04060 Cytokine-cytokine receptor |
| | | inflammation | interaction |
| | | | 04640 Hematopoietic cell lineage |
| | | | 04659 Th17 cell differentiation |
| | | | 04750 Inflammatory mediator |
| | | | regulation of TRP channels |
| | | | 04380 Osteoclast differentiation |
| | | | 04050 Cytokine receptors |
| | | | 04090 CD molecules |
| ENO2 (Gamma-enolase) | 2026 | Carbon and glycan biosynthesis | 00010 Glycolysis/Gluconeogenesis |
| | | metabolism | 03018 RNA degradation |
| | | | 04066 HIF-1 signaling pathway |
| | | | 03019 Messenger RNA biogenesis |
| | | | 04147 Exosome |
| CTSH (Cathepsin H) | 1512 | Proteolysis | 04142 Lysosome |
| | | | 04210 Apoptosis |
| | | | 01002 Peptidases and inhibitors |
| ESD (S-formylglutathione | 2098 | Carbon and glycan biosynthesis | hsa01200 Carbon metabolism |
| hydrolase) | | metabolism | |
| RPS27A (Ubiquitin + 1, | 6233 | Genetic information processing | 03010 Ribosome |
| truncated mutation for UbB) | | | 04147 Exosome |
| UBE2N (Ubiquitin-conjugating | 7334 | Proteolysis | 04120 Ubiquitin mediated proteolysis |
| enzyme E2 N) | | | 04121 Ubiquitin system |
| | | | 03400 DNA repair and recombination |
| | | | proteins |
| GAPDH (Glyceraldehyde-3- | 2597 | Carbon and glycan biosynthesis | 00010 Glycolysis/Gluconeogenesis |
| phosphate dehydrogenase) | | metabolism | 04066 HIF-1 signaling pathway |
| | | | 04131 Membrane trafficking |
| | | | 04147 Exosome |
| MMP8 (Neutrophil | 4317 | Proteolysis | 01002 Peptidases and inhibitors |
| collagenase) | | | |
| IFNL1 (Interferon lambda-1) | 282618 | Signal transduction | 04630 Jak-STAT signaling pathway |
| | | | 04060 Cytokine-cytokine receptor |
| | | | interaction |
| | | | 04052 Cytokines and growth factors |
| KIF23 (Kinesin-like protein | 9493 | Genetic information processing | 04131 Membrane trafficking |
| KIF23) | | | 03036 Chromosome and associated |
| | | | proteins |
| | | | 04812 Cytoskeleton proteins |
| IL3RA (Interleukin-3 receptor | 3563 | Signal transduction, | 04630 Jak-STAT signaling pathway |
| subunit alpha) | | Development and regeneration | 04151 PI3K-Akt signaling pathway |
| | | | 04060 Cytokine-cytokine receptor |
| | | | interaction |
| | | | 04210 Apoptosis |
| | | | 04640 Hematopoietic cell lineage |
| | | | 04050 Cytokine receptors |
| | | | 04090 CD molecules |
| VIP (Vasoactive Intestinal | 7432 | Signal transduction, Feeding | 04024 cAMP signaling pathway |
| Peptide) | | behavior | 04080 Neuroactive ligand-receptor |
| | | | interaction |
| FLRT1 (Leucine-rich repeat | 23769 | Signal transduction | 99995 Signaling proteins |
| transmembrane protein | | | |
| FLRT1) | | | |
| DNAJC19 (Mitochondrial | 131118 | Genetic information processing | 03110 Chaperones and folding |
| import inner membrane | | | catalysts |
| translocase subunit TIM14) | | | 03029 Mitochondrial biogenesis |
| FGF8 (Fibroblast growth factor | 2253 | Signal transduction | 04014 Ras signaling pathway |
| 8 isoform A) | | | 04015 Rap1 signaling pathway |
| | | | 04010 MAPK signaling pathway |
| | | | 04151 PI3K-Akt signaling pathway |
| | | | 04810 Regulation of actin |
| | | | cytoskeleton |
| | | | 04052 Cytokines and growth factors |
| | | | 00536 Glycosaminoglycan binding |
| | | | proteins |
| PRKCI (Protein kinase C iota | 5584 | Signal transduction, Platelet | 04015 Rap1 signaling pathway |
| type) | | activation and blood | 04390 Hippo signaling pathway |
| | | coagulation, Insulin signaling | 04144 Endocytosis |
| | | pathway | 04530 Tight junction |
| | | | 04611 Platelet activation |
| | | | 04910 Insulin signaling pathway |
| | | | 01001 Protein kinases |
| PSMA6 (Proteasome subunit | 5687 | Proteolysis | 03050 Proteasome |
| alpha type-6) | | | 01002 Peptidases and inhibitors |
| HDGFRP2 (Hepatoma-derived | 84717 | — | — |
| growth factor-related protein 2) | | | |
| RPS6KA5 (Ribosomal protein | 9252 | Signal transduction | 04010 MAPK signaling pathway |
| S6 kinase alpha-5) | | | 04668 TNF signaling pathway |
| | | | 04261 Adrenergic signaling in |
| | | | cardiomyocytes |
| | | | 04722 Neurotrophin signaling |
| | | | pathway |
| | | | 04713 Circadian entrainment |
| | | | 01001 Protein kinases |
| FGF6 (Fibroblast growth | 2251 | Signal transduction | 04014 Ras signaling pathway |
| factor 6) | | | 04015 Rap1 signaling pathway |
| | | | 04010 MAPK signaling pathway |
| | | | 04151 PI3K-Akt signaling pathway |
| | | | 04810 Regulation of actin |
| | | | cytoskeleton |
| | | | 04052 Cytokines and growth factors |
| | | | 00536 Glycosaminoglycan binding |
| | | | proteins |
| CAMKK1 (Calcium/calmodulin- | 84254 | Signal transduction | 05034 Alcoholism |
| dependent protein kinase | | | 01001 Protein kinases |
| kinase 1) | | | |
| CRTAM (Cytotoxic and | 56253 | Signal transduction | 04090 CD molecules |
| regulatory T-cell molecule) | | | |
| ULBP3 (NKG2D ligand 3) | 79465 | Innate immune response | 04650 Natural killer cell mediated |
| | | | cytotoxicity |
| DHH (Desert hedgehog protein | 50846 | Signal transduction, Proteolysis, | 04340 Hedgehog signaling pathway |
| N-product) | | Development and regeneration | 01002 Peptidases and inhibitors |
| LGALS4 (Galectin-4) | 3960 | Lectins | 04091 Lectins |
| RAN (GTP-binding nuclear | 5901 | Genetic information processing | 03013 RNA transport |
| protein Ran) | | | 03008 Ribosome biogenesis in |
| | | | eukaryotes |
| | | | 03019 Messenger RNA biogenesis |
| | | | 03009 Ribosome biogenesis |
| | | | 03016 Transfer RNA biogenesis |
| | | | 03036 Chromosome and associated |
| | | | proteins |
| | | | 04147 Exosome |
| | | | 04031 GTP-binding proteins |
| ACE2 (Angiotensin-converting | 59272 | Proteolysis | 04614 Renin-angiotensin system |
| enzyme 2) | | | 04974 Protein digestion and |
| | | | absorption |
| | | | 01002 Peptidases and inhibitors |
| | | | [BR: hsa01002] |
| | | | 04147 Exosome |
| AK1 (Adenylate kinase | 203 | Nucleotide metabolism | 00230 Purine metabolism |
| isoenzyme 1) | | | 00730 Thiamine metabolism |
| | | | 04147 Exosome |
| MBD4 (Methyl-CpG-binding | 8930 | Genetic information processing | 03410 Base excision repair |
| domain protein 4) | | | 03036 Chromosome and associated |
| | | | proteins |
| | | | 03400 DNA repair and recombination |
| | | | proteins |
| S100A6 (Protein S100-A6) | 6277 | — | — |
| FCRL3 (Fc receptor-like | 115352 | Signal transduction | 04090 CD molecules |
| protein 3) | | | |

TABLE 26
Monosaccharide and glycosidic linkage composition of fiber preparations
(A) Monosaccharide content (%/Σ sugars)

| | Monosaccharide content (%/Σ sugars) | | | | | | | |
|---|---|---|---|---|---|---|---|---|
| Purified fiber preparation | Glc | GalA | Ara | Xyl | Gal | Man | Rha | Fuc |
| Pea fiber | 51.5 | 13.9 | 22.4 | 6.2 | 3.3 | 0.6 | 1.9 | 0.1 |
| Orange fiber (coarse) | 26.4 | 42.9 | 13.9 | 3.5 | 6.5 | 4.1 | 2.3 | 0.4 |
| Barley bran | 80.6 | 0 | 7.1 | 9.9 | 0.7 | 1.2 | 0.5 | 0 |
| Pea fiber (extruded) | 60.1 | 11.9 | 18.3 | 4.4 | 3.0 | 0.8 | 1.5 | 0 |
| Orange fiber (extruded) | 40.7 | 35.4 | 9.8 | 3.0 | 4.8 | 3.9 | 2.1 | 0.3 |

Abbreviations: glucose (Glc), galacturonic acid (GalA), arabinose (Ara), xylose (Xyl),
galactose (Gal), mannose (Man), rhamnose (Rha), fucose (Fuc)

(B) Deduced glycosyl-linkage (%/Σ linked-sugars)

| | | Orange fiber | | Pea fiber | Orange fiber |
|---|---|---|---|---|---|
| | Pea fiber | (coarse) | Barley bran | (extruded) | (extruded |
| T-Glc(p) | 1.3 | 0 | 3.0 | 2.6 | 1.5 |
| 3-Glc(p) | 0 | 0 | 5.2 | 0 | 0 |
| 4-Glc(p) | 46.5 | 13.0 | 71.3 | 51.7 | 49.4 |
| 3,4-Glc(p) | 0.4 | 0 | 0 | 0.6 | 0.3 |
| 4,6-Glc(p) | 1.7 | 0 | 3.4 | 3.2 | 1.9 |
| 2,3,4,6-Glc(p) | 0.2 | 0 | 1.5 | 0 | 0.1 |
| T-GalA(p) | 0 | 0.1 | 0 | 0 | 0 |
| T-GalA(p)-methyl ester | 0 | 0.4 | 0 | 0 | 0 |
| 4-GalA(p) | 6.8 | 24.0 | 0 | 3.7 | 6.3 |
| 4-GalA(p)-methyl ester | 0.7 | 31.5 | 0 | 0.5 | 11.1 |
| 3,4-GalA(p) | 0.9 | 0.4 | 0 | 0.5 | 0 |
| 3,4-GalA(p)-methyl ester | 0 | 0.2 | 0 | 0.2 | 0 |
| 4,6-GalA(p) | 0 | 0.1 | 0 | 0 | 0 |
| 4,6-GalA(p)-methyl ester | 0 | 0.4 | 0 | 0 | 0 |
| T-Ara(f) | 9.4 | 2.4 | 1.6 | 13.3 | 8.8 |
| 5-Ara(f) | 12.2 | 8.3 | 0 | 10.4 | 8.5 |
| 2,5-Ara(f) | 3.5 | 0 | 0 | 2.8 | 0 |
| 3,5-Ara(f) | 1.4 | 4.7 | 0 | 0.7 | 2.8 |
| T-Xyl(p) | 1.3 | 0 | 0 | 2.8 | 0 |
| 4-Xyl(p) | 6.9 | 2.4 | 3.0 | 2.8 | 3.2 |
| 3,4-Xyl(p) | 0 | 0 | 0.9 | 0 | 0 |
| 2,3,4-Xyl(p) | 0 | 0 | 2.4 | 0 | 0 |
| T-Gal(p) | 0.5 | 0.9 | 0 | 0.4 | 0.5 |
| 3-Gal(p) | 1.5 | 1.2 | 0 | 0.5 | 0.7 |
| 4-Gal(p) | 3.0 | 8.1 | 1.0 | 2.3 | 3.7 |
| 2,3,4-Gal(p) | 0.1 | 0 | 0 | 0 | 0 |
| 4,6-Gal(p) | 0 | 0.2 | 0 | 0 | 0 |
| 4,6-Man(p) | 0 | 0 | 0 | 0 | 0.2 |
| T-Rha(p) | 0.1 | 0 | 0 | 0 | 0 |
| 2-Rha(p) | 0.4 | 0.9 | 0 | 0.5 | 0.7 |
| 2,4-Rha(p) | 0.8 | 0 | 0 | 0.5 | 0 |
| 2,4-Hex | 0 | 0 | 1.1 | 0 | 0 |
| 3,4-Hex | 0 | 0 | 2.9 | 0 | 0 |
| 2,3,4-Hex | 0 | 0 | 0.9 | 0 | 0 |
| 3,4,6-Hex | 0 | 0 | 1.7 | 0 | 0 |

Abbreviations: glucose (Glc), galacturonic acid (GalA), arabinose (Ara), xylose (Xyl), galactose (Gal),
mannose (Man), rhamnose (Rha), fucose (Fuc), hexose (Hex), terminal (T), pyranose (p), furanose (f)

TABLE 27
Description of humans with obesity whose fecal microbial community
samples were used in the gnotobiotic animal experiments

| Subject | Race/ | | H | W | | Insulin | Glc | | | | Total | | hs- |
|---|---|---|---|---|---|---|---|---|---|---|---|---|---|
| ID | ethnicity | Age | (m) | (kg) | BMI | Basal | Basal | H-IR | LDL | HDL | chol. | TG | CRP |
| TP01-01 | White | 41 | 1.6 | 104.6 | 38.8 | 25.3 | 5.2 | 5.9 | 138.0 | 34.0 | 213.0 | 204.0 | 5.2 |
| | (Caucasian) | | | | | | | | | | | | |
| TP02-01 | White | 38 | 1.6 | 83.2 | 34.6 | 16.0 | 3.6 | 2.5 | 88.0 | 53.0 | 189.0 | 241.0 | 3.9 |
| | (Caucasian) | | | | | | | | | | | | |
| TP03-02 | Black or | 36 | 1.5 | 111.9 | 46.6 | 13.1 | 5.0 | 2.9 | 77.0 | 66.0 | 163.0 | 101.0 | 16.0 |
| | African | | | | | | | | | | | | |
| | American | | | | | | | | | | | | |
| TP04-01 | White | 33 | 1.7 | 114.6 | 38.7 | 13.9 | 5.0 | 3.1 | 135.0 | 44.0 | 203.0 | 122.0 | 4.6 |
| | (Caucasian) | | | | | | | | | | | | |
| TP05-02 | White | 32 | 1.8 | 104.3 | 34.0 | 12.1 | 4.1 | 2.2 | 97.0 | 47.0 | 164.0 | 101.0 | 3.4 |
| | (Caucasian) | | | | | | | | | | | | |
| TP06-01 | White | 37 | 1.7 | 119.0 | 41.9 | 20.2 | 4.2 | 3.8 | 77.0 | 40.0 | 135.0 | 88.0 | 9.9 |
| | (Caucasian) | | | | | | | | | | | | |
| TP07-02 | White | 37 | 1.6 | 82.6 | 32.7 | 7.3 | 4.6 | 1.5 | 108.0 | 80.0 | 199.0 | 56.0 | 1.6 |
| | (Caucasian) | | | | | | | | | | | | |
| TP08-02 | White | 41 | 1.7 | 92.4 | 32.6 | 9.9 | 4.5 | 2.0 | 103.0 | 45.0 | 165.0 | 87.0 | 6.0 |
| | (Caucasian) | | | | | | | | | | | | |
| TP09-02 | White | 38 | 1.6 | 104.8 | 39.3 | 21.9 | 5.1 | 5.0 | 97.0 | 33.0 | 145.0 | 74.0 | 2.8 |
| | (Caucasian) | | | | | | | | | | | | |

Abbreviations:
height (H),
weight (W),
cholesterol (chol),
triglycerides (TG),
Glucose (Glc),
Body mass index (BMI),
Homeostatic model asssessment of insulin resistance (H-IR),
low-density lipoproteins (LDL),
high-density lipoproteins (HDL),
high-sensitivity C-reactive protein (hs-CRP)
BMI values are kg/m²
LDL, HDL, Total chol, and TG values are in mg/dL
hs-CRP values are in mg/L
Insulin values are in μU/mL, and Glucose values are in mmol/L.

TABLE 28
Description of subjects enrolled human study 1 (pea fiber snack food prototype)

| Subject | | Race/ | | Height | Weight | BMI | HOMA- |
|---|---|---|---|---|---|---|---|
| ID | Sex | ethnicity | Age | (cm) | (kg) | (kg/m²) | IR |
| S.01 | M | Asian | 31 | 167.9 | 74.6 | 26.5 | 2.6 |
| S.02 | F | White (Caucasian) | 38 | 161.3 | 85.5 | 32.9 | 2.0 |
| S.03 | M | White (Caucasian) | 38 | 174.9 | 86.8 | 28.4 | 3.5 |
| S.05 | M | Black or African American | 47 | 172.5 | 98.3 | 33.0 | 1.6 |
| S.06 | F | Black or African American | 47 | 158.5 | 68.3 | 27.2 | 1.1 |
| S.07 | M | White (Caucasian) | 34 | 179.0 | 102.0 | 31.8 | 2.0 |
| S.09 | F | Black or African American | 38 | 163.4 | 89.1 | 33.4 | 7.3 |
| S.11 | F | Black or African American | 51 | 158.5 | 72.5 | 28.9 | 3.0 |
| S.12 | F | Black or African American | 34 | 154.0 | 65.3 | 27.5 | 3.6 |
| S.13 | M | Black or African American | 32 | 177.0 | 102.3 | 32.7 | 3.6 |
| S.14 | M | White (Caucasian) | 48 | 182.5 | 113.9 | 34.2 | 2.2 |
| S.15 | M | Black or African American | 55 | 188.5 | 98.4 | 27.7 | 0.3 |

Abbreviations:
body mass index (BMI),
homeostatic model assessment of insulin resistance (HOMA-IR)

TABLE 29
Description of subjects enrolled in enrolled in human study 2 (pea fiber plus inulin
snack prototype, and pea fiber, inulin, orange fiber and barley bran snack prototype)

| Subject | | Race/ | | Height | Weight | BMI | HOMA- |
|---|---|---|---|---|---|---|---|
| ID | Sex | ethnicity | Age | (cm) | (kg) | (kg/m²) | IR |
| S.02 | F | White (Caucasian) | 39 | 162.5 | 80.0 | 30.3 | 2.5 |
| S.03 | M | White (Caucasian) | 39 | 175.6 | 80.7 | 26.2 | 1.0 |
| S.05 | M | Black or African American | 47 | 172.1 | 99.4 | 33.6 | 3.0 |
| S.06 | F | Black or African American | 48 | 158.7 | 67.6 | 26.8 | 0.9 |
| S.09 | F | Black or African American | 38 | 162.0 | 90.1 | 34.3 | 7.3 |
| S.11 | F | Black or African American | 51 | 158.6 | 71.6 | 28.5 | 2.0 |
| S.12 | F | Black or African American | 34 | 155.0 | 65.5 | 27.3 | 2.5 |
| S.13 | M | Black or African American | 32 | 177.8 | 106.2 | 33.6 | 6.7 |
| S.14 | M | White (Caucasian) | 48 | 181.5 | 115.7 | 35.1 | 2.8 |
| S.110 | M | White (Caucasian) | 34 | 182.8 | 87.3 | 26.1 | 1.8 |
| S.113 | F | White (Caucasian) | 38 | 164.0 | 69.7 | 25.9 | 0.8 |
| S.114 | F | White (Caucasian) | 59 | 159.8 | 85.8 | 33.6 | 4.4 |
| S.122 | F | White (Caucasian) | 27 | 167.5 | 76.3 | 27.2 | 1.7 |
| S.123 | F | White (Caucasian) | 26 | 170.0 | 75.1 | 26.0 | 2.7 |

Abbreviations:
body mass index (BMI),
homeostatic model assessment of insulin resistance (HOMA-IR)

### References for Examples 12-17

1. NCD Risk Factor Collaboration Trends in adult body-mass index in 200 countries from 1975 to 2014: a pooled analysis of 1698 population-based measurement studies with 19.2 million participants. Lancet 387, 1377-1396 (2016).
2. GBD 2017 Diet collaborators, health effects of dietary risks in 195 countries, 1990-2017: a systematic analysis for the global burden of disease study Lancet 393, 1958-1972. (2017).
3. M. A. Conlon, A. R. Bird, The impact of diet and lifestyle on gut microbiota and human health. Nutrients 7, 17-44 (2014).
4. A. M. Fernstrand, D. Bury, J. Garssen, J. C. Verster, Dietary intake of fibers: differential effects in men and women on perceived general health and immune functioning. Food Nutr. Res. 61 (2017).
5. V. M. Gershuni, E. S. Friedman, S. A. Ogawa, C. Tanes, K. Bittinger, G. D. Wu, Su2002—Alterations in the small bowel microbiota and metabolome, induced by the interaction between dietary fat and fiber, are associated with the host metabolic phenotype. Gastroenterology 156, S-687 (2019).
6. C. L. Bodinham, L. Smith, J. Wright, G. S. Frost, M. D. Robertson, Dietary fibre improves first-phase insulin secretion in overweight individuals. PLoS One 7, e40834 (2012).
7. H. Hauner, A. Bechthold, H. Boeing, A. Brönstrup, A. Buyken, E. Leschik-Bonnet, J. Linseisen, M. Schulze, D. Strohm, G. Wolfram, Evidence-Based guideline of the German Nutrition Society: Carbohydrate intake and prevention of nutrition-related diseases. Ann. Nutr. Metab. 60, 1-58 (2012).
8. M. Sleeth, A. Psichas, G. Frost, Weight gain and insulin sensitivity: a role for the glycaemic index and dietary fibre? Br. J. Nutr. 109, 1539-1541(2013).
9. L. Zhao, F. Zhang, X. Ding, G. Wu, Y. Y. Lam, X. Wang, H. Fu, X. Xue, C. Lu, J. Ma, L. Yu, C. Xu, Z. Ren, Y. Xu, S. Xu, H. Shen, X. Zhu, Y. Shi, Q. Shen, W. Dong, R. Liu, Y. Ling, Y. Zeng, X. Wang, Q. Zhang, J. Wang, L. Wang, Y. Wu, B. Zeng, H. Wei, M. Zhang, Y. Peng, C. Zhang, Gut bacteria selectively promoted by dietary fibers alleviate type 2 diabetes. Science 359, 1151-1156 (2018).
10. M. S. Desai, A. M. Seekatz, N. M. Koropatkin, N. Kamada, C. A. Hickey, M. Wolter, N. A. Pudlo, S. Kitamoto, N. Terrapon, A. Muller, V. B. Young, B. Henrissat, P. Wilmes, T. S. Stappenbeck, G. Nunez, E. C. Martens, A dietary fiber-deprived gut microbiota degrades the colonic mucus barrier and enhances pathogen susceptibility. Cell 167, 1339-1353.e21 (2016).
11. C. Menni, M. A. Jackson, T. Pallister, C. J. Steves, T. D. Spector, A. M. Valdes, Gut microbiome diversity and high-fibre intake are related to lower long-term weight gain. Int. J. Obesity 41, 1099-1105 (2017).
12. E. D. Sonnenburg, S. A. Smits, M. Tikhonov, S. K. Higginbottom, N. S. Wingreen, J. L. Sonnenburg, Diet-induced extinctions in the gut microbiota compound over generations. Nature 529, 212-215 (2016).
13. P. Vangay, A. J. Johnson, T. L. Ward, G. A. Al-Ghalith, R. R. Shields-Cutler, B. M. Hillmann, S. K. Lucas, L. K. Beura, E. A. Thompson, L. M. Till, R. Batres, B. Paw, S. L. Pergament, P. Saenyakul, M. Xiong, A. D. Kim, G. Kim, D. Masopust, E. C. Martens, C. Angkurawaranon, R. McGready, P. C. Kashyap, K. A. Culhane-Pera, D. Knights, US immigration westernizes the human gut microbiome. Cell 175, 962-972 (2018).
14. V. K. Ridaura, J. J. Faith, F. E. Rey, J. Cheng, A. E. Duncan, A. L. Kau, N. W. Griffin, V. Lombard, B. Henrissat, J. R. Bain, M. J. Muehlbauer, O. Ilkayeva, C. F. Semenkovich, K. Funai, D. K. Hayashi, B. J. Lyle, M. C. Martini, L. K. Ursell, J. C. Clemente, W. Van Treuren, W. A. Walters, R. Knight, C. B. Newgard, A. C. Heath, J. I. Gordon, Gut microbiota from twins discordant for obesity modulate metabolism in mice. Science 341, 1241214 (2013).
15. M. L. Patnode, Z. W. Beller, N. D. Han, J. Cheng, S. L. Peters, N. Terrapon, B. Henrissat, S. Le Gall, L. Saulnier, D. K. Hayashi, A. Meynier, S. Vinoy, R. J. Giannone, R. L. Hettich, J. I. Gordon, Interspecies competition impacts targeted manipulation of human gut bacteria by fiber-derived glycans. Cell 179, 59-73 (2019).
16. N. Terrapon, V. Lombard, E. Drula, P. Lapebie, S. Al-Masaudi, H. J. Gilbert, B. Henrissat, PULDB: the expanded database of Polysaccharide Utilization Loci. Nucleic Acids Res 46, D677-D683 (2018).
17. R. K. Aziz, D. Bartels, A. A. Best, M. DeJongh, T. Disz, R. A. Edwards, K. Formsma, S. Gerdes, E. M. Glass, M. Kubal, F. Meyer, G. J. Olsen, r. Olson, A. L. Osterman, R. A. Overbeek, L. K. McNeil, D. Paarmann, T. Paczian, B. Parrello, G. D. Pusch, C. Reich, R. Stevens, O. Vassieva, V. Vonstein, A. Wilke, O. Zagnitko, The RAST Server: rapid annotations using subsystems technology. BMC Genomics 9, 75 (2008).
18. R. Overbeek, T. Begley, R. M. Butler, J. V. Choudhuri, H.-Y. Chuang, M. Cohoon, V. de Crécy-Lagard, N. Diaz, T. Disz, R. Edwards, M. Fonstein, E. D. Frank, S. Gerdes, E. M. Glass, A. Goesmann, A. Hanson, D. Iwata-Reuyl, R. Jensen, N. Jamshidi, L. Krause, M. Kubal, N. Larsen, B. Linke, A. A. McHardy, F. Meyer, H. Neuweger, G. Olsen, R. Olson, A. Osterman, V. Portnoy, G. D. Pusch, D. A. Rodionov, C. Ruckert, J. Steiner, r. Stevens, I. Thiele, O. Vassieva, Y. Ye, O. Zagnitko, V. Vonstein, The subsystems approach to genome annotation and its use in the project to annotate 1000 genomes. Nucleic Acids Res. 33, 5691-5702 (2005).
19. D. A. Rodionov, A. A. Arzamasov, M. S. Khoroshkin, S. N. Iablokov, S. A. Leyn, S. N. Peterson, P. S. Novichkov, A. L. Osterman, A. L. Micronutrient requirements and sharing capabilities of the human gut microbiome. Front. Microbiol. 10, 1316 (2019).
20. E. D. Sonnenburg, H. Zheng, P. Joglekar, S. K. Higginbottom, S. J. Firbank, D. N. Bolam, J. L. Sonnenburg, Specificity of polysaccharide use in intestinal Bacteroides species determines diet-induced microbiota alterations. Cell 141, 1241-1252 (2010).
21. S. C. A. de Jager, B. W. C. Bongaerts, M. Weber, A. O. Kraaijeveld, M. Rousch, S. Dimmeler, M. P. van Dieijen-Visser, K. B. J. M. Cleutjens, P. J. Nelemans, T. J. C. van Berkel, E. A. Biessen, Chemokines CCL3/MIP1α, CCLS/RANTES and CCL18/PARC are independent risk predictors of short-term mortality in patients with acute coronary syndromes. PLoS One 7 (2012).
22. A. C. Shore, H. M. Colhoun, A. Natali, C. Palombo, F. Khan, G. Östling, K. Aizawa, C. Kennbäck, F. Casanova, M. Persson, K. Gooding, P. E. Gates, H. Looker, F. Dove, J. Belch, S. Pinnola, E. Venture, M. Kozakova, I. Goncalves, J. Kravic, H. Bjorkbacka, J. Nilsson, Use of vascular assessments and novel biomarkers to predict cardiovascular events in type 2 diabetes: The SUMMIT VIP Study. Diabetes Care 41, 2212-2219 (2018).
23. M. J. Barratt, C. Lebrilla, H.-Y. Shapiro, J. I. Gordon, The gut microbiota, food science, and human nutrition: A timely marriage. Cell Host Microbe 22, 134-141 (2017).
24. J. M. Green, M. J. Barratt, M. Kinch, J. I. Gordon, J. I. Food and microbiota in the FDA regulatory network. Science 357, 39-40 (2017)
25. K. K. Bucholz, A. C. Heath, P. A. Madden, Transitions in drinking in adolescent females: evidence from the missouri adolescent female twin study. Alcohol Clin. Exp. Res. 24, 914-923 (2000).
26. M. D. Mifflin, S. T. St Joer, L. A. Hill, B. J. Scott, S. A. Daugherty, Y. O. Koh, A new predictive equation for resting energy expenditure in healthy individuals. Am. J. Clin. Nutr. 51, 241-7 (1990).
27. A. F. Subar, F. E. Thompson, V. Kipnis, D. Midthune, P. Hurwitz, S. McNutt, A. McIntosh, S. Rosenfeld, Comparative validation of the Block, Willett, and National Cancer Institute food frequency questionnaires: the Eating at America's Table Study. Am. J. Epidemiol. 154, 1089-99 (2001).

### Methods for Examples 12-17

(a) Gnotobiotic Mouse Studies

Husbandry—To test the effects of different fiber preparations on uncultured human fecal microbial communities, adult germ-free male C57BL/6J mice (12-16-weeks-old) were dually-housed in plastic cages located in plastic flexible film gnotobiotic isolators (Class Biologically Clean Ltd., Madison, Wis.). Mice were maintained at 23° C. under a strict 12 h light cycle (lights on at 0600 h). Cages contained autoclaved paper ‘shepherd shacks’ to facilitate their natural nesting behaviors and to provide environmental enrichment.

Diets—The HiSF-LoFV diet was milled to powder (D90 particle size, 980 μm), and mixed with powdered fiber preparations [10% (w/w)]. Fiber content was defined for each preparation [Association of Official Agricultural Chemists (AOAC) 2009.01]. Similarly, protein, fat, total carbohydrate, ash, and water content were measured [protein AOAC 920.123; fat AOAC 933.05; ash AOAC 935.42; moisture AOAC 926.08; total carbohydrate (100−(Protein+Fat+Ash+Moisture)]. The powdered food mixtures were vacuum-packed in sterile plastic containers and sterilized by gamma irradiation (20-50 kilograys, Steris, Mentor, Ohio). Sterility was confirmed by culturing the diets under aerobic and anaerobic conditions (atmosphere, 75% N₂, 20% CO₂, 5% H₂) at 37° C. in TYG medium.

Transplantation of human fecal microbiota into germ-free mice—A 500 mg aliquot of a pulverized frozen fecal sample that had been obtained from nine unrelated obese adult female members of the Missouri Adolescent Female Twin Study (MOAFTS) cohort (25) listed in Table 27 was diluted in 5 mL of reduced PBS [1×PBS supplemented with 0.1% Resazurin (w/v), 0.05% L-cysteine-HCl] in an anaerobic Coy chamber (atmosphere, 75% N2, 20% CO₂, 5% Ha). The sample was vortexed for 2 minutes at room temperature in 5 mL of 2 mm-diameter autoclaved borosilicate glass beads in order to disrupt clumps of bacterial cells trapped within the fecal matrix. The resulting suspension was filtered through a sterile nylon mesh cell strainer (100 μM pore diameter; BD Falcon). The filtrate was then mixed with 5 mL of sterile PBS containing 0.1% Resazurin (w/v), 0.05% L-cysteine-HCl, and 30% (v/v) glycerol, transferred to a sterile glass crimped tube and stored at −80° C. until further use. Aliquots of the stored filtrate were transported in a frozen state to the gnotobiotic mouse facility. The outer surface of the tube was sterilized by a 30-minute exposure to chlorine dioxide in the transfer sleeve attached to the gnotobiotic isolator, and then introduced into the isolator.

Diet oscillation studies—Germ-free C57BL/6J mice were weaned onto and subsequently maintained on an autoclaved, low-fat, high-plant polysaccharide chow (catalog number 2018S, Envigo) that was administered ad libitum. Four days prior to colonization, mice were switched to a diet low in saturated fats and high in fruits and vegetables (LoSF-HiFV) that was formulated based on the National Health and Nutrition Examination Survey of US dietary practices (14). A 300 μL aliquot of a clarified suspension of a given fecal microbiota sample was introduced into the stomachs of 12-16-week-old male mice using an oral gavage needle. Recipients were maintained in separate gnotobiotic isolators dedicated to animals colonized with the same donor microbiota. Four days after gavage, mice were switched to the HiSF-LoFV diet (14,15).

Mice in the experimental groups completed a 64-day multi-phase diet-oscillation feeding protocol. On day 4 after colonization, animals were fed a pelleted version of the HiSF-LoFV diet (14,15) for 10 days. Beginning on experimental day 14, mice were fed 20-30 g aliquots of a dough-like diet/fiber mixture that was made from the milled HiSF-LoFV diet supplemented with 10% (w/w) raw pea fiber (Pea fiber EF 100; J. Rettenmaier & SOhne GmbH & Co. KG) and hydrated with 10-15 mL sterile water (mixing of the sterile powdered diet and sterile water occurred within the gnotobiotic isolator). The resulting dough-like mixture was pressed into a plastic feeding dish and placed on the cage floor for feeding ad libitum. Food supply was monitored daily, and a freshly hydrated aliquot of the diet was supplied every 3 days to prevent food levels from dropping below roughly one third of the original volume. The HiSF-LoFV/pea fiber mixture was administered for 10 d after which time mice were returned to the unsupplemented pelleted HiSF-LoFV diet for 10 d (‘wash out period’). On day 34, mice were given 20-30 g aliquots of a diet/fiber mixture made from the milled HiSF-LoFV diet supplemented with 10% (w/w) coarse orange fiber (CitriFi 100; Fiber Star, Inc.). The HiSF-LoFV/orange fiber mixture was administered for 10 days. Mice were then returned to the unsupplemented pelleted HiSF-LoFV diet for 10 days. On experimental day 54, mice began receiving 20-30 g aliquots of the diet/fiber mixture made from the powdered HiSF-LoFV diet supplemented with 10% (w/w) raw barley bran fiber (Barley Balance—concentrated (1-3)(1-4) β-glucan; PolyCell Technologies, LLC). The HiSF-LoFV/barley bran fiber mixture was administered for 10 days. All animals were euthanized by cervical dislocation without prior fasting on experimental day 64.

Bedding (Aspen Woodchips; Northeastern Products) was replaced after each 10-day diet oscillation period to prevent any leftover food or fecal matter from being ingested during the following diet change. Fresh fecal samples were collected from each animal into sterile cryo-resistant polypropylene tubes within seconds of being produced on day 4 after initial colonization while consuming the LoSF-HiFV diet, and on days 5 and 10 of each 10-day oscillation period. Samples were placed in liquid nitrogen 45-60 min after they were collected. Pre-colonization fecal samples were also collected to verify the germ-free status of mice (by culture and by bacterial V4-16S rDNA amplicon sequencing).

(b) Human Studies

Subjects provided written, informed consent before participating in these studies. The first study (ClinicalTrials.gov NCT04159259) was performed between February and July, 2019. The second study (ClinicalTrials.gov NCT04101344) was conducted between August and December, 2019.

Study 1 Design—A total of 18 men and women who were overweight or obese (BMI≥25.0 and ≤35.0 kg/m²), aged≥18 and ≤60 years, were screened for potential participation in this study. Subjects completed a comprehensive medical evaluation, including a medical history, physical examination, assessment of food preferences and aversions, and standard blood tests. A fecal sample was collected during the medical evaluation phase and used to determine whether Bacteroides species were present in their microbiota (B. vulgatus, B. thetaiotaomicron, B. cellulosilyticus, B. uniformis, and/or B. ovatus). Subjects whose fecal microbiota contained less than 0.1% relative abundance of B. vulgatus (defined by V4 16S rDNA amplicon sequencing), and less than 0.1% relative abundance of at least one of the other Bacteroides. were excluded from the study. Additional exclusion criteria included: (i) history of previous bariatric surgery; (ii) significant organ system dysfunction (e.g., diabetes, severe pulmonary, kidney, liver or cardiovascular disease); (iii) history of inflammatory gastrointestinal disease; (iv) pregnant or lactating; (v) use of medications known to affect the study outcome measures that could not be temporarily discontinued; (vi) use during the month prior to screening of medications known to affect the composition of the gut microbiota (e.g., antibiotics); (vii) bowel movements<3 times per week; (viii) vegans, vegetarians, those with lactose intolerance and/or severe allergies/aversions/sensitivities to foods and ingredients included in the prescribed meal plan; and (ix) individuals who were not able to grant voluntary informed consent. Of 18 participants who were screened, four were excluded based on the Bacteroides criterion and two were excluded based on the screening assessment. Each of the 12 subjects who participated in the study completed the study per protocol.

The study design is described in FIG. 31A. Participants who met the enrollment criteria were asked to maintain their usual eating habits for 4 days with fecal samples collected at home on days 1, 2, 3 and 4. On days 5 through day 14, participants consumed only HiSF-LoFV meals (see below) that were provided by the study team in the form of packed-out meals. Fecal samples were collected at home on study days 6, 8, 10, 12 and 14 and a fasting blood sample was collected on day 14. Ten days after starting the HiSF-LoFV diet, subjects supplemented their diet with the pea fiber-containing snack (see below and Table 21), starting with 1 bar per day on day 15 and 16 (with lunch), 2 bars per day on day 17 and 18 (one at lunch and one at dinner) and 3 bars per day from day 19 through 35 (at breakfast, lunch and dinner). All at-home fecal samples during the ramp up period (i.e., days 15-20) were collected and subsequently, at-home fecal samples every two days (i.e., on study days 21, 23, 25, 27, 29, 31, 33 and 35), with fasting blood collected on day 29 and day 35. After completing 17 consecutive days consuming 3 snacks per day (24.3 g of pea fiber), participants returned to consuming the unsupplemented HiSF-LoFV diet for an additional 14 days, with fecal samples collected every two days (study days 37, 39, 41, 43, 45, 47 and 49) and fasting blood collected on day 49.

Study 2 Design—A total of 23 men and women who were overweight or obese were screened for potential participation in this study by using the same exclusion/exclusion criteria as Study 1 with the exception that there was no prescreen for the representation of Bacteroides species in subjects' fecal samples. Among the 23 participants who completed screening, 19 were enrolled and 14 completed the study protocol. Five participants did not complete the intervention for personal reasons unrelated to the study intervention. The study design is described in FIG. 35B. On day 1, participants provided a fecal sample prior to entering the controlled diet phase of the study. From day 2-11, participants consumed HiSF-LoFV meals provided by the study team in the form of packed-out meals and snacks; they collected at-home fecal samples on study days 5, 9, 10 and 11. A fasting blood sample was obtained on day 11. On day 12, subjects began supplementing their diet with the two-fiber snack prototype [1 snack serving per day on day 12 (with lunch), 2 bars per day on day 13 (one at lunch and one at dinner) and 3 servings per day on day 14 through 25 (at breakfast, lunch and dinner)]. Participants collected all at home fecal samples during the ramp up period (i.e., days 12-14) and subsequently, at-home fecal samples on days 18, 23, 24 and 25. A fasting blood sample was collected on day 25 just prior to returning to the unsupplemented HiSF-LoFV diet (days 26-35). During this ‘wash-out’ phase of the study, fecal samples were collected on days 28, 33, 34 and 35 and a fasting blood sample on day 35. Consumption of the four-fiber snack prototype began on day 36 [1 snack serving per day on day 36 (with lunch), 2 snacks per day on day 37 (one at lunch and one at dinner) and 3 snacks per day on day 38 through 49 (at breakfast, lunch and dinner)]. Subjects collected all at-home fecal samples during the ramp-up period (days 36-38) and subsequently, at-home fecal samples on days 41, 47, 48 and 49. A fasting blood sample was obtained on day 49 (last day of study).

Fiber snacks—Snack food prototypes were prepared by Mondelēz International, Inc., tested to confirm the absence of microbial contamination/pathogens, and then shipped to and stored at Washington University. Research participants received weekly shipments of the snack food prototypes. The composition of these prototypes is described in Table 21A. Their organoleptic properties were designed based on common USA consumer preferences.

Design, manufacture and distribution of HiSF-LoFV diets—Participants consumed a diet composed of approximately 40% fat, 20% protein, and 40% carbohydrate that was high in saturated fat and low in fruits and vegetables during the dietary intervention. The HiSF-LoFV diet is high in refined grains (white bread and pasta, bagels, and corn cereals), added sugars (sugar-sweetened beverages, candies, and desserts), vegetables sourced primarily from potatoes and tomatoes, and protein and fat derived from animals. Representative diets are shown in table 6B. Each participant's estimated energy requirements were calculated using the Mifflin St. Jeor equation (26) multiplied by an appropriate physical activity level (PAL). To ensure consistent intake of nutrients across all participants and ensure weight stability, a registered dietitian designed a seven-day cycle menu specific to the participant's energy needs and instructed each participant to consume only foods prescribed by the study team during the dietary intervention. The energy provided was adjusted, as needed, to ensure subjects remained weight stable throughout. All food was provided in the form of packed-out meals and snacks prepared by the metabolic kitchen in the Clinical Translational Research Unit (CTRU) at Washington University.

Collection of clinical meta-data—Subjects were provided with electronic smart scales (BodyTrace, Inc.) to enable weight monitoring between study visits. At enrollment, habitual dietary patterns were assessed using the National Cancer Institute Diet History Questionnaire III (DHQIII) food frequency questionnaire (27). Subjects visited the CTRU on a weekly basis to pick up packed-out meals (using insulated bags and rolling coolers), have their body weight measured, and any changes to their health and medications reviewed. During the study, participants recorded all food and beverage intake using a web-based food diary during all diet phases. An experienced study dietitian trained study participants on how to complete the food records and reviewed these records with the participants at each study visit to ensure the accuracy of self-reported data. In addition, a member of the study team contacted participants regularly to (i) check on study progress, (ii) discuss prescribed and non-prescribed foods and beverages consumed, (iii) discuss weight changes, and (iv) ensure participants have sufficient fecal collection kits.

Preparation of blood samples—Fasting blood samples were obtained in the CTRU. Conventional blood chemistry tests were performed by the Clinical Laboratory Improvement Amendments (CLIA)-certified Core Laboratory for Clinical Studies (CLCS) at Washington University School of Medicine. To prepare plasma for SOMAscan proteomics analysis (SomaLogic, Boulder, Colo.), blood samples (10-20 mL) were aliquoted into EDTA-K2 treated tubes and centrifuged at 2,000×g for 10 minutes at 4° C. Following centrifugation, plasma was immediately transferred into cryo-resistant polypropylene tubes (0.5 mL aliquots), de-identified, and stored at −80° C. prior to analysis according to manufacturer's recommendations.

(c) Fecal Sample Collection, Processing and Culture-Independent Analyses

Sample collection and processing—Participants collected fecal samples using small medically approved collection containers. Participants were provided with a freezer (−20° C.) at the beginning of the study for temporary storage of fecal samples. Containers were labeled with a unique study identifier to protect subject confidentiality, and the collection date and time. Each sample was frozen immediately at −20° C. and shipped in a frozen state (using frozen gel packs). Samples were shipped on a regular basis to a biospecimen repository in Washington University in St. Louis where they were stored at −80° C. until the time of processing. Fecal samples were homogenized with a porcelain mortar (4 L) and pestle while submerged in liquid nitrogen. Multiple 500 mg aliquots of the pulverized frozen material were prepared and stored at −80° C.

b. DNA was extracted from an aliquot of each pulverized human fecal sample (˜50-100 mg) or mouse fecal pellets (˜20-50 mg) by first bead-beating (BioSpec Mini-beadbeater-96,) for 4 minutes in 250 μL of 0.1 mm-diameter zirconium oxide beads and a 3.97 mm-diameter steel ball in a solution consisting of 500 μL buffer A (200 mM NaCl, 200 mM Trizma base, 20 mM EDTA), 210 μL of 20% SDS, and 500 μL phenol:chloroform:isoamyl alcohol (25:24:1), followed by centrifugation at 3,220×g for 4 minutes. DNA was purified (QiaQuick 96 purification kit; Qiagen, Valencia, Calif.), eluted in 130 μL of 10 mM Tris-HCl pH 8.5 (buffer EB, Qiagen), and quantified (Quant-iT dsDNA broad range kit; Invitrogen). Purified DNA was stored at −20° C. for further processing.

16S rDNA amplicon sequencing and identification of ASVs—Purified DNA samples were adjusted to a concentration of 1 ng/μL and subjected to PCR using barcoded primers directed against variable region 4 of the bacterial 16S rRNA gene (28). PCR amplification was performed using the following cycling conditions: denaturation (94° C. for 2 minutes), 26 cycles of 94° C. for 15 seconds, 50° C. for 30 seconds and 68° C. for 30 seconds, and incubation at 68° C. for 2 minutes. Amplicons with sample-specific barcodes were quantified, pooled and sequenced (Illumina MiSeq instrument, paired-end 250 nt reads).

Paired-end reads were demultiplexed, trimmed to 200 nucleotides, merged, and chimeras removed using the 1.13.0 version of the DADA2 pipeline (29) in R (v. 3.6.1). Amplicon sequence variants (ASV) generated from DADA2 were aligned against the GreenGenes 2016 (v. 13.8) reference database to 97% sequence identity, followed by taxonomic and species assignment with RDP 16 (release 11.5) and SILVA (v. 128). The resulting ASV table was filtered to include only ASVs with ≥0.1%, relative abundance in at least five samples and rarefied to 15,000 reads/sample.

Results were also obtained with another approach to taxonomic assignment. In this procedure, each representative sequence is aligned (NCBI BLAST toolkit version 2.10.0) to a 16S rRNA gene reference database compiled by joining unique sequences from Ribosomal Database Project (RDP) version 11.5 and the NCBI 16S ribosomal RNA Project. Alignment results are sorted based on percentage of sequence identity, with maximum values denoted as “M”. Hits are selected with identities in the range [M] to [M−(1−M)/S] where “S” is scaling parameter that controls the maximum number of taxonomic descriptors accepted for a ‘multi-taxonomic assignment’ (MTA) based on 16S rDNA sequence identity (in this study, set to 4) (30).

Shotgun sequencing and annotation of microbiomes—Purified DNA samples were adjusted to a concentration of 0.75 ng/μL. Sequencing libraries were generated from each DNA sample using the Nextera DNA Library Prep Kit (Illumina) with the reaction volume scaled down 10-fold to 2.5 μL (31). Samples were pooled and sequenced with Illumina NextSeq 550 instrument in the case of all mouse samples [10.7±0.6×10⁶ paired-end 150 nucleotide-long reads/sample (mean±s.d.)] and all human samples in Study 2 (12.8±1.2×10⁶ paired-end 150 nucleotide-long reads/sample), while an Illumina NovaSeq Model 6000 instrument was used to sequence human samples collected during the course of Study 1 (28.0±4.2 10⁶ paired-end 150 nucleotide-long reads/sample).

After sequencing, reads were demultiplexed (bcl2fastq, Illumina), adapter sequences were trimmed using cutadapt (32) and reads were quality filtered with Sickle (33). Human and mouse DNA sequences were identified, and removed using Bowtie2 (34) and either the hg19 build of the H. sapiens genome or the Mus musculus C57BL/6J strain genome (UCSC mm10), depending on sample type, prior to further processing. Host-filtered reads were assembled using IDBA-UD (35) and annotated with prokka (36) Gene counts were generated by mapping quality-controlled, paired-end reads generated from each sample to the corresponding assembled contigs. Duplicate reads (optical- and PCR-generated) were identified and removed from mapped data using the Picard MarkDuplicates tool (v 2.9.3). Mapping results were processed to generate count data (featureCounts; Subread v. 1.5.3 package) (37) and normalized (transcripts per kilobase million reads, TPM) in R (v. 3.4.1; 38).

The genomic integration platform SEED, which is a growing repertoire of complete and nearly complete microbial genomes with draft annotations performed by the RAST server (39), was used for additional annotations of fecal microbiomes. Functional profiles for each fecal microbiome were generated by assigning microbiome-encoded proteins to microbial community SEED (mcSEED) metabolic pathways/modules that capture core metabolism of nutrients/metabolites in four major categories (amino acids, sugars, fermentation products and vitamins) projected over ˜2,600 reference bacterial genomes (19). Protein sequences from prokka-annotated (36) fecal DNA assemblies were queried against representative protein sequences from the mcSEED subsystems/pathway modules using DIAMOND (40) with a threshold of ≥80%, identity for best hits. Microbiome-encoded proteins were assigned the best-hit annotation of the representative mcSEED protein.

CAZymes annotations were performed for the full set of open reading frames identified by prokka. Assignment to CAZyme families was performed using a custom script which, in a first step, compared each amino acid sequence to the full-length sequences listed in the CAZy database (download date Apr. 21, 2020) using Blastp (version 2.3.0+) (41). Sequences giving e-values worse than 10⁻⁴ were discarded while sequences showing 100% coverage with an e-value of at least 10⁻⁶ and more than 50% identity with a sequence already in the CAZy database were directly assigned to the same family (or families in the case of modular proteins) as the subject sequence. All other sequences were subjected to a second, parallel, similarity search using two methods; (i) Blastp against a library of sequences corresponding to the individual modules in the CAZy database, and (ii) HMMER3 (42) using a collection of custom-made HMMs built after the CAZy families (and subfamilies for families GH5, 13, 16, 30 and 43). Assignments were kept when the two methods gave the same results with >90% overlap and an e-value better than 10⁻⁴ for all families except for carbohydrate esterases (threshold 10⁻²⁰), and non-LPMO auxiliary activities (threshold 10⁻²⁵). These various thresholds were designed to eliminate as much as possible oxidoreductases or esterases not specific for carbohydrates and to give results more consistent with the manual procedure used for updates of the CAZy database.

(d) Quantitative Proteomics of Human Plasma Samples

Levels of 1305 proteins were quantified in a 50 mL aliquot of plasma using the SOMAscan 1.3K Proteomic Assay plasma/serum kit (SomaLogic, Boulder, Colo.). Procedures used for quality control filtering and analysis of differential protein abundances are described in ref 43. Briefly, microarrays were scanned with an Agilent SureScan instrument at 5 □m resolution and the Cy3 fluorescence readout was quantified. Raw fluorescence signal values from each SOMAmer reagent were processed using standardization procedures that are recommended by the manufacturer (i.e., datasets were normalized to remove hybridization variation within a run followed by median normalization across all samples to remove other assay biases). The final adat file was log₂-transformed, quantile-normalized and then filtered to remove non-human SOMAmer reagents. A total of 1205 and 1170 proteins were then used for downstream analyses of participants in human studies 1 and 2, respectively.

(e) Higher-Order Singular Value Decomposition

FIG. 30B illustrates SVD. The result of performing SVD on matrix M is creation of three matrices; U, E, and V. U is a matrix of cleft singular vectors' (LSVs), V is a matrix of ‘right singular vectors’ (RSVs), and E is a matrix only of diagonal values (‘singular values’). The k^{th} element of E relates the k^{th} left and right singular vectors. A new matrix can be created that considers the information contained within a single singular value. For example, FIG. 30B shows that multiplying left singular vector 1 (LSV1), singular value 1 (SV1), and right singular vector 1 (RSV1) creates a new matrix M¹ that has the same dimensions as matrix M but exclusively contains information within singular vector 1. Higher-order (HO)-SVD is used when the input matrix has more than two degrees of freedom (FIG. 30B). Mathematically, these types of matrices are called ‘tensors.’ Unlike SVD, HO-SVD is not a technique with an analytical solution; i.e., a tensor of rank N cannot be written as a product of N+1 tensors as in the case with SVD. As a consequence, several methods of approximation exist to deconstruct higher-order tensors for feature-reduction purposes. ‘Canonical Polyadic decomposition’ (CP decomposition), deconstructs a tensor into a sum of rank-1 tensors (arrays) related to each other through a ‘core tensor’. FIG. 30B shows the result of CP decomposition on a three-dimensional tensor O. The core tensor, G, is a three-dimensional tensor comprised of only diagonal elements, each of which specifies the amount of variance carried by a ‘tensor component’ analogous to the singular values computed by SVD. Each tensor component (TC) relates the rows, columns, and third-dimensional entries of O. The number of tensor components is determined by creating a tensor that is randomized with respect to the rows, columns, and third dimension entries and performing CP decomposition over 100 trials. The randomization process scrambles the correlations between each dimension of the tensor; therefore, the resulting CP decomposition reflects a random distribution of tensor component values. We used the alternating least squares algorithm for the CP decomposition (CP-ALS) to iteratively improve the matrix factorization. The lowest tensor component whose variance is above that of tensor component 1 of the scrambled tensor defines the number of tensor components considered.

(f) Over Representation Analysis of HO-SVD Discriminatory Plasma Proteins

A list of discriminatory plasma proteins, defined as being in the 20th percentile of the most positive and negative projections along HO-SVD-defined tensor components was mapped to the Kyoto Encyclopedia of Genes and Genomes (KEGG) pathway database (44-46) and tested for functional enrichment analysis using clusterProfiler in R (47). An over-representation analysis employing a hypergeometric test was used to identify KEGG pathways enriched during consumption of each fiber snack prototype. A list of all plasma proteins measured by SOMAscan that passed quality control criteria (1205 for human study 1 and 1170 for human study 2) was used as the background list of plasma proteins for the over representation pathway analysis (parameters: organism=“hsa”, keyType=“kegg”, pAdjustMethod=“BH”, minGSSize=5, maxGSSize=500). A combined list of plasma proteins identified as significantly enriched in insulin and glucagon signaling pathways for all three fiber snack prototypes was used in the heatmaps shown in FIG. 33.

(g) Cross-Correlation Singular Value Decomposition Analysis

CC-SVD begins by computing the cross-correlation matrix between two feature types. Given two matrices of dimensions N^{m×n} (with elements N_{i,j}) and P^{m×p} (with elements P_{k,l}) where m is the number of samples and n and p are the number of features of each feature type, a cross-correlation matrix is calculated by taking each feature in the m×n matrix N and correlating them with each feature in the m×p matrix P. The resulting matrix is a n×p cross-correlation matrix C^{n×p}, where each element C_{1,i} contains the correlation between feature N_{1:m,j} from the first matrix and feature P_{1:m,l} from the second matrix (note that these starting matrices contain abundance information whereas the resulting cross-correlation matrix contains correlations between features). Next, SVD is used to decompose the cross-correlation matrix C into left and right singular matrices which contain left and right singular vectors (SVs), respectively; the left SVs correspond to the features of N and the right SVs correspond to the features of P. An SV represents a module of cross-correlated features with a unique correlation profile, and the projections of each feature onto an SV represents the module membership of that feature (e.g., how similar a feature's correlation profile is to the overall module's correlation profile). To define a module, a user-defined threshold truncates the leading and trailing tails of the distribution of projections along an SV, and the features above and below the truncation are considered module members. Note that SVD determines a projection for all features along each SV, providing a continuous measure of module membership. Because the input matrix decomposed by SVD is a correlation matrix, features with large positive projections on a left SV will be strongly correlated with features with large positive projections on the matching right SV and negatively correlated with features with large negative projections on the matching left SV. Concordantly, features with large negative projections on a left SV will be strongly correlated with features with large negative projections on the right SV and negatively correlated with features with large positive projections on the left SV. The number of SVs that should be considered modules is determined using a random-matrix approximation described elsewhere (48). Module members are selected from the original cross-correlation matrix C and plotted using the ‘corrplot’ function in R (49) in rank order by their projections onto an SV, with larger magnitude projection values indicating a correlation pattern similar to the module's overall correlation profile. The continuous nature of projection values enabled us to rank-order proteins by their projections along SV1 and to use the KEGG database (44-46) to identify and relate biological processes to CAZyme-associated proteins.

(h) Mass Spectrometry-Based Carbohydrate Analysis of Fibers, Diets and Fecal Samples

Monosaccharide and linkage analysis of fiber preparations—Methods described in ref. 15 were used to define the carbohydrate composition of the pea, orange and barley bran fiber preparations. Following a pre-hydrolysis step (incubation in concentrated sulfuric acid (72%) for 30 minutes at 30° C. to release glucose from cellulose), the fibers were treated with 1 M sulfuric acid for 6 hours at 100° C. Individual neutral sugars were analyzed by gas chromatography as their alditol acetate derivatives (50,51). The metahydroxydiphenyl colorimetric acid method was used to measure uronic acid (as galacturonic acid) (52,53); sodium tetraborate was used to differentiate glucuronic acid from galacturonic acid (54). Galacturonic acid (pectins) methylation was estimated according to ref. 55.

Linkage analysis of fibers followed procedures detailed in ref. 56 with minor modifications that allowed for discrimination of galactose, galacturonic acid, and methyl-esterified galacturonic acid. Briefly, reduction of carboxymethyl ester groups of uronic acids was performed with NaBD₄ and imidazole-HCl, followed by activation of carboxylic acid groups with carbodiimide and a second reduction with imidazole-HCl, NaBH₄ (D/H) and NaBD₄ (D/D). Samples were dialyzed, freeze-dried and then solubilized in DMSO before methylation with iodomethane of the accessible hydroxyl groups of reduced polysaccharides. Acid hydrolysis with trifluoroacetic acid and a subsequent reduction with NaBD₄ of partially methylated sugars was performed. Lastly, samples were acetylated, extracted as partially methylated alditol acetates (PMAA) into dichloromethane, and analyzed by gas chromatography-mass spectrometry (GC-MS) (57).

Homogenization of mouse diets and fecal biospecimens—For homogenization of HiSF-LoFV diet with and without fiber supplementation, a 10 mg/mL stock solution was prepared from frozen starting material. Pre-weighed mouse and human fecal samples were diluted 10-fold in Nanopure water (Thermo Fisher) and homogenized overnight. Samples were then centrifuged and a 200 μL aliquot of the supernatant was taken for metabolomic analysis, while the remaining material was lyophilized to complete dryness and diluted to create a stock solution (10 mg/mL water). Stock solutions were bullet-blended using 1.4 mm stainless steel beads followed by incubation at 100° C. for 1 h. Lastly, samples were subjected to another bullet blend process and aliquots were taken for monosaccharide and linkage analysis.

Monosaccharide analysis—Methods for monosaccharide analysis of diets and fecal samples were adapted from ref. 58. Briefly, three 10 μL aliquots were taken from each bullet-blended ‘stock’, transferred to a 96-well plate and subjected to acid hydrolysis (4 M trifluoroacetic acid for 1 h at 121° C.). The reaction was quenched with 855 μL of ice-cold Nanopure water. Hydrolyzed samples were derivatized with 1-phenyl-3-methyl-5-pyrazolone (PMP) according to conditions described in ref. 59. Samples and 14 monosaccharide standards (0.001-100 μg/mL) were reacted in 0.2 M PMP (prepared in methanol) and 28% NH₄OH at 70° C. for 30 minutes. Derivatized glycosides were then dried to completion (vacuum centrifuge) and reconstituted in Nanopure water. Excess PMP was removed (chloroform extraction) and a 1 μL aliquot of the aqueous layer was injected into an Agilent 1290 infinity II UHPLC coupled to an Agilent 6495A triple quadrupole mass spectrometer under dMRM mode. Monosaccharides were quantified using an external calibration curve.

Linkage analysis—The procedure for linkage analysis was adapted from previously described protocols. In short, three replicate 5 μL aliquots of each bullet-blended stock solution were incubated in saturated NaOH and iodomethane (in DMSO) to achieve methylation of free hydroxyl groups. Excess NaOH and DMSO were removed by extraction with dichloromethane and water. Permethylated samples were subsequently hydrolyzed and derivatized (using the same procedure employed for monosaccharide analysis). Derivatized samples were subjected for ultra-high-performance liquid chromatography-multiple reaction monitoring-mass spectrometry. Glycosidic linkages present in samples were identified using a pool of oligosaccharide standards and a comprehensive linkage library described elsewhere (60,61).

LC-QTOF-MS identification of a fecal biomarker of orange fiber consumption—Methods for preparing samples and performing LC-QTOF-MS using an Agilent 1290 LC system coupled to an Agilent 6545 Q-TOF mass spectrometer are detailed in an earlier publication (62). Five μL of each prepared fecal sample for positive ESI ionization were injected into a BEH C18 column (2.1×150 mm, 1.7 μm, Waters Corp.) that was heated to 35° C. The mobile phase was 0.1% formic acid in water (A) and 0.1% formic acid in acetonitrile (B). The following gradient was applied at a flow rate of 0.3 ml/min over 14 minutes; 95% A/5% B to 100% B, followed by 3 minutes at 100% B.

### References for Methods for Examples 1-6

28. J. G. Caporaso, C. L. Lauber, W. A. Walters, D. Berg-Lyons, C. A. Lozupone, P. J. Turnbaugh, N. Fierer, R. Knight, Global patterns of 16S rRNA diversity at a depth of millions of sequences per sample. Proc. Natl. Acad. Sci. U.S.A. 108 Suppl 1, 4516-4522 (2011).
29. B. J. Callahan, P. J. McMurdie, M. J. Rosen, A. W. Han, A. J. A. Johnson, S. P. Holmes, DADA2: High-resolution sample inference from Illumina amplicon data. Nat. Methods 13, 581-583 (2016).
30. B. Di Luccia, P. P. Ahern, N. W. Griffin, J. Cheng, J. L. Guruge, A. E. Byrne, D. A. Rodionov, S. A. Leyn, A. L. Osterman, T. Ahmed, M. Colonna, M. J. Barratt, N. F. Delahaye, J. I. Gordon, Combined prebiotic and microbial intervention improves oral cholera vaccination responses in a mouse model of childhood undernutrition. Cell Host Microbe 27, 1-10 (2020).
31. M. Baym, S. Kryazhimskiy, T. D. Lieberman, H. Chung, M. M. Desai, R. Kishony, Inexpensive multiplexed library preparation for megabase-sized genomes. PloS One 10, e0128036 (2015).
32. M. Martin, Cutadapt removes adapter sequences from high-throughput sequencing reads. EMBnet. J. 17, 10-12 (2011).
33. N. A. Joshi, J. N. Fass, Sickle: A sliding-window, adaptive, quality-based trimming tool for FastQ files. (Version 1.33) (2011).
34. B. Langmead, S. L. Salzberg, Fast gapped-read alignment with Bowtie 2. Nat. Methods 9, 357-359 (2012).
35. Y. Peng, H. C. M. Leung, S. M. Yiu, F. Y. L. Chin, IDBA-UD: a de novo assembler for single-cell and metagenomic sequencing data with highly uneven depth. Bioinformatics 28, 1420-1428 (2012).
36. T. Seemann, Prokka: rapid prokaryotic genome annotation. Bioinformatics 30, 2068-2069 (2014).
37. Y. Liao, G. K. Smyth, W. Shi, FeatureCounts: an efficient, general purpose program for assigning sequence reads to genomic features. Bioinformatics 30, 923-930 (2014).
38. R Core Team R: A language and environment for statistical computing. R Foundation for Statistical Computing, Vienna, Austria. URL www.R-project.org/(2017).
39. R. Overbeek, R. Olson, G. D. Pusch, G. J. Olsen, J. J. Davis, T. Disz, R. A. Edwards, S. Gerdes, B. Parrello, M. Shukla, The SEED and the rapid annotation of microbial genomes using subsystems technology (RAST). Nucleic Acids Res. 42, D206-214 (2014).
40. B. Buchfink, C. Xie, D. H. Huson, Fast and sensitive protein alignment using DIAMOND. Nat. Methods 12, 59-60 (2015).
41. C. Camacho, G. Coulouris, V. Avagyan, N. Ma, J. Papadopoulos, K. Bealer, T. L. Madden, T. L. BLAST+: Architecture and Applications. BMC Bioinformatics 10, 421 (2009).
42. J. Mistry, R. D. Finn, S. R. Eddy, A. Bateman, M. Punta, Challenges in homology search: HMMER3 and convergent evolution of coiled-coil regions. Nucleic Acids Res. 41, e121 (2013).
43. Y. Chen, V. L. Kung, D. Subhashish, S. Hossain, M. C. Hibberd, J. Guruge, M. Mahfuz, K. N. Begum, M. M. Rahman, S. M. Fahim, Md. A. Gazi, M. R. Hague, S. A. Sarker, R. N. Mazunder, B. Di Luccia, K. Ahsan, E. Kennedy, J. Santiago-Borges, D. A. Rodionov, S. A. Leyn, A. L. Osterman, M. J. Barratt, T. Ahmed, J. I. Gordon, Duodenal microbiota in stunted undernourished children with enteropathy. New Engl. J. Med. (2020).
44. M. Kanehisa, S. Goto, KEGG: kyoto encyclopedia of genes and genomes. Nucleic Acids Res. 28, 27-30 (2000).
45. M. Kanehisa, Y. Sato, M. Furumichi, K. Morishima, M. Tanabe, New approach for understanding genome variations in KEGG. Nucleic Acids Res. 47, D590-D595 (2019).
46. M. Kanehisa, Toward understanding the origin and evolution of cellular organisms. Protein Sci. 28, 1947-1951 (2019).
47. G. Yu, L.-G. Wang, Y. Han, Q.-Y. He, clusterProfiler: an R package for comparing biological themes among gene clusters. OMICS. 16, 284-287 (2012).
48. V. Plerou, P. Gopikrishnan, B. Rosenow, L. A. Amaral, T. Guhr, H. E. Stanley, Random matrix approach to cross correlations in financial data. Phys. Rev. E. Stat. Nonlin Soft Matter Phys. 65, 1-18 (2002).
49. T. Wei, V. R. Simko, package “corrplot”: Visualization of a correlation matrix. (2017).
50. A. B. Blakeney, P. J. Harris, R. J. Henry, B. A. Stone, A simple and rapid preparation of alditol acetates for monosaccharide analysis. Carbohydrate Res. 113, 291-299 (1983).
51. H. N. Englyst, J. H. Cummings, Improved method for measurement of dietary fiber as non-starch polysaccharides in plant foods. J. Assoc. Off. Anal. Chem. 71, 808-814 (1988).
52. N. Blumenkrantz, G. Asboe-Hansen, New method for quantitative determination of uronic acids. Anal. Biochem. 54, 484-489 (1973).
53. J.-F. Thibault, Automatisation du dosage des substances pectiques par la méthode au métahydroxydiphényle. Lebensm. Wiss. Technol. 12, 247-251 (1979).
54. T. M. C. C. Filisetti-Cozzi, N. C. Carpita, Measurement of uronic acids without interference from neutral sugars. Analytical Biochem. 197, 157-162 (1991).
55. S. Levigne, M. Thomas, M.-C. Ralet, B. Quemener, J.-F. Thibault, Determination of the degrees of methylation and acetylation of pectins using a C18 column and internal standards. Food Hydrocolloids 16, 547-550 (2002).
56. F. A. Pettolino, C. Walsh, G. B. Fincher, A. Bacic, Determining the polysaccharide composition of plant cell walls. Nature Protocols 7, 1590-1607 (2012).
57. F. Buffetto, V. Cornuault, M. G. Rydahl, D. Ropartz, C. Alvarado, V. Echasserieau, S. Le Gall, B. Bouchet, O. Tranquet, Y. Verhertbruggen, W. G. T. Willats, J. P. Knox, M.-C. Ralet, F. Guillon, The deconstruction of pectic rhamnogalacturonan I unmasks the occurrence of a novel arabinogalactan oligosaccharide epitope. Plant Cell Physiol. 56, 2181-2196 (2015).
58. M. J. Amicucci, A. G. Galermo, E. Nandita, T.-T. T. Vo, Y. Liu, M. Lee, G. Xu, C. B. Lebrilla, A rapid-throughput adaptable method for determining the monosaccharide composition of polysaccharides. Int. J. Mass Spectrom. 438, 22-28 (2019).
59. G. Xu, M. J. Amicucci, Z. Cheng, A. G. Galermo, C. B. Lebrilla, Revisiting monosaccharide analysis—quantitation of a comprehensive set of monosaccharides using dynamic multiple reaction monitoring. The Analyst 143, 200-207 (2017).
60. A. G. Galermo, E. Nandita, M. Barboza, M. J. Amicucci, T.-T. T. Vo, C. B. Lebrilla, Liquid chromatography-tandem mass spectrometry approach for determining glycosidic linkages. Anal. Chem. 90, 13073-13080 (2018).
61. A. G. Galermo, E. Nandita, J. J. Castillo, M. J. Amicucci, C. B. Lebrilla, Development of an extensive linkage library for characterization of carbohydrates. Anal. Chem. 91, 13022-13031 (2019).
62. C. A. Cowardin, P. P. Ahern, V. L. Kung, M. C. Hibberd, J. Cheng, J. L. Guruge, V. Sundaresan, R. D. Head, D. Barile, D. A. Mills, M. J. Barratt, S. Huq, T. Ahmed, J. I. Gordon, Mechanisms by which sialylated milk oligosaccharides impact bone biology in a gnotobiotic mouse model of infant undernutrition, Proc. Natl. Acad. Sci USA 116, 11988-11996 (2019).

What is claimed is:

1. A fiber blend comprising
at least 15 wt % of one or more pea fiber preparation or a glycan equivalent thereof; and
at least one additional fiber preparation chosen from
at least 28 wt % of one or more high molecular weight inulin preparation or a glycan equivalent thereof,
between 0 wt % and 10 wt %, inclusive, of one or more citrus pectin preparation or a glycan equivalent thereof,
between 0 wt % and 25 wt %, inclusive, of one or more citrus fiber preparation or a glycan equivalent thereof, or
between 0 wt % and 45 wt %, inclusive, of one or more barley fiber preparation or a glycan equivalent thereof.

2. The fiber blend of claim 1, which comprises at least 28 wt % of one or more pea fiber preparation, or a glycan equivalent thereof.

3. The fiber blend of claim 2, which comprises at least 30 wt % of one or more pea fiber preparation, or a glycan equivalent thereof; and there is at least 30 wt % of one or more high molecular weight inulin preparation, or a glycan equivalent thereof.

4. The fiber blend of claim 1, which comprises less than 1 wt % of one or more citrus pectin preparation, or a glycan equivalent thereof.

5. The fiber blend of claim 1, which comprises no citrus pectin preparation, or a glycan equivalent thereof.

6. The fiber blend of claim 1, which comprises 15 wt % or less of one or more citrus fiber preparation or a glycan equivalent thereof, or 12 wt % or less of one or more citrus fiber preparation or a glycan equivalent thereof.

7. The fiber blend of claim 6, wherein the citrus fiber preparation is an orange fiber preparation.

8. The fiber blend of claim 1, which comprises is 25 wt % or less of one or more barley fiber preparation or glycan equivalent thereof, or 20 wt % or less of one or more barley fiber preparation, or glycan equivalent thereof.

9. (canceled)

10. The fiber blend of claim 1, which comprises
about 25 wt % to about 40 wt % of one or more pea fiber preparation or a glycan equivalent thereof, about 5 wt % to about 15 wt % of one or more citrus fiber preparation or a glycan equivalent thereof, about 30 wt % to about 40 wt % of a high molecular weight inulin preparation or glycan equivalent thereof, about 10 wt % to about 30 wt % of a barley fiber preparation or glycan equivalent thereof; or
about 30 wt % to about 40 wt % of one or more pea fiber preparation or a glycan equivalent thereof, about 10 wt % to about 20 wt % of one or more citrus fiber preparation or a glycan equivalent thereof, about 30 wt % to about 40 wt % of a high molecular weight inulin preparation or glycan equivalent thereof, about 15 wt % to about 25 wt % of a barley fiber preparation or glycan equivalent thereof; or
about 55 wt % to about 65 wt % of one or more pea fiber preparation or a glycan equivalent thereof and about 30 wt % to about 40 wt % of a high molecular weight inulin preparation or glycan equivalent thereof; or
about 60 wt % to about 70 wt % of one or more pea fiber preparation or a glycan equivalent thereof and about 30 wt % to about 40 wt % of a high molecular weight inulin preparation or glycan equivalent thereof; or
about 60 wt % to about 65 wt % of one or more pea fiber preparation or a glycan equivalent thereof and about 35 wt % to about 40 wt % of a high molecular weight inulin preparation or glycan equivalent thereof.

11. The fiber blend of claim 10, which comprises less than 1 wt % of one or more citrus pectin preparation, or a glycan equivalent thereof.

12. The fiber blend of claim 10, wherein the citrus fiber preparation is an orange fiber preparation.

13. (canceled)

14. A food composition comprising a fiber blend of claim 1.

15. The food composition of claim 14, wherein the food compostions is a baked, pressed or extruded food composition.

16. The food composition of claim 14, wherein the fiber blend is about 30 wt % to about 50 wt % of the food composition.

17. The food composition of claim 14, wherein the fiber blend provides about 30% or more of the total dietary fiber in the food composition or about 50% or more of the total dietary fiber in the food composition.

18. (canceled)

19. The food composition of claim 14, wherein the food composition further comprises flour(s), meal(s), oil(s), fat(s), inclusions, sweetener(s), starch(es), salt(s), emulsifier(s), leavening agent(s), preservative(s) or combinations thereof.

20.-29. (canceled)

30. The food composition of claim 19, wherein the food composition further comprises a color additive, a flavor, a flavor enhancer, a stabilizer, a humectant, a firming agent, an enzyme, a probiotic, a spice, a binder, fruit, vegetables, grains, vitamins, minerals or combinations thereof.

31.-37. (canceled)

38. The food composition of claim 14, wherein administration of the food composition at least once daily for a minimum of five days to a subject increases the abundance of one or more member of at least one CAZyme family measured in a fecal sample obtained from the subject.

39. The food composition of claim 38, wherein the one or more member of at least one CAZyme family is selected from the group consisting of α-L-arabinofuranosidase (GH43_33), β-galactosidase (GH147), N-acetylmuramidase (GH108), endo-1,2,-α-mannanase (GH99), and β-glucosidase (GH116).

40.-45. (canceled)

46. The food composition of claim 14, wherein administration of the food composition at least once daily for a minimum of five days to a subject consuming a Western diet reduces weight gain in the subject, as measured against a population of similar subjects consuming a Western diet without administration of the food composition.
